(19) United States
(12) Patent Application Publication
(10) Pub. No.: US 2024/0417729 A1
(43) Pub. Date: Dec. 19, 2024

(54) THERAPEUTIC TREATMENT FOR FRAGILE X-ASSOCIATED DISORDER

(71) Applicant: University of Massachusetts, Boston, MA (US)

(72) Inventors: Joel D. Richter, Wellesley, MA (US); Sneha Shah, Worcester, MA (US); Jonathan Watts, Worcester, MA (US)

(21) Appl. No.: 18/751,096

(22) Filed: Jun. 21, 2024

Related U.S. Application Data
(63) Continuation-in-part of application No. PCT/US2022/082380, filed on Dec. 23, 2022.
(60) Provisional application No. 63/265,989, filed on Dec. 23, 2021.
(60) Provisional application No. 63/649,322, filed on May 17, 2024.

(51) Int. Cl. C12N 15/113

(52) U.S. Cl. CPC C12N 15/113; C12N 2310/11; C12N 2310/315; C12N 2310/321

(57) ABSTRACT

Provided herein, in various embodiments, are methods of treating a fragile X-associated disorder (e.g., fragile X syndrome), comprising administering to a subject in need thereof, a therapeutically effective amount of an agent that decreases expression of an aberrant fragile X messenger ribonucleoprotein 1 (FMR1) gene product (e.g., FMR1-217). Also provided herein, in various embodiments, are compositions (e.g., polynucleotides such as antisense oligonucleotides or pharmaceutical compositions) for decreasing expression of an aberrant FMR1 gene product.

### RELATED APPLICATIONS

This application is a continuation-in-part of International Application No. PCT/US2022/082380, filed on Dec. 23, 2022, which designates the United States, published in English, and claims the benefit of U.S. Provisional Application No. 63/265,989, filed on Dec. 23, 2021. This application also claims the benefit of U.S. Provisional Application No. 63/649,322, filed on May 17, 2024. The entire teachings of the above applications are incorporated herein by reference.

### GOVERNMENT SUPPORT

This invention was made with government support under GM135087, GM046779 and NS111990 from the National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
a) File name: 54391028-003_SL.xml; created Jun. 21, 2024, 155,162 Bytes in size.

### BACKGROUND

Fragile X syndrome (FXS) is an autism spectrum disorder that is the most frequent inherited form of intellectual impairment. FXS afflicts 1 in 4,000 boys and 1 in 7,000 girls. In addition to intellectual impairment, children with FXS present a range of symptoms including speech and developmental delays, perseveration, hyperactivity, aggression, and epilepsy, among other maladies. FXS is caused by a CGG triplet repeat expansion in a single gene, fragile X messenger ribonucleoprotein 1 (FMR1), which resides on the X chromosome. When the CGG triplet expands to 200 or more, the FMR1 gene is methylated and thereby transcriptionally inactivated. The loss of the FMR1 gene product, the protein fragile X messenger ribonucleoprotein (FMRP), is the cause of the disorder.

Treatments for fragile X syndrome (and other autism spectrum disorders), which are mostly based on animal models, have met with very limited success in human clinical trials (Hagerman et al., Fragile X syndrome, Nature Rev Disease Primers 3:17065 (2017); Berry-Kravis et al., Drug development for neurodevelopmental disorders: lessons learned from fragile X syndrome, Nature Rev Drug Disc. 17:280-299 (2018)). Indeed, there is no widely applicable therapy that shows even modest efficacy for FXS.

### SUMMARY

There is a critical need to develop methods and therapeutic agents for treating fragile X-associated disorders such as fragile X syndrome (FXS). The disclosure provides such methods and therapeutic agents.

The disclosure provided herein is based, in part, on the discovery that, in FXS cells, antisense oligonucleotide (ASO) treatment reduces the expression of the CGG expansion-dependent aberrantly spliced FMR1-217 RNA and restores fragile X messenger ribonucleoprotein (FMRP) to levels observed in cells from typically developing individuals. Accordingly, the disclosure generally relates to compositions (e.g., polynucleotides, pharmaceutical compositions) and methods that are useful for treating a fragile X-associated disorder.

In one aspect, the present disclosure provides a method of treating a fragile X-associated disorder, comprising administering to a subject in need thereof, a therapeutically effective amount of an agent that decreases expression of an aberrant fragile X messenger ribonucleoprotein 1 (FMR1) gene product, thereby treating the fragile X-associated disorder in the subject.

In another aspect, the present disclosure provides a method of treating a fragile X-associated disorder, comprising administering to a subject in need thereof, a therapeutically effective amount of an agent that modulates splicing of an FMR1 gene (e.g., decreasing splicing between Exons 1 and 2 of FMR1-217), thereby treating the fragile X-associated disorder in the subject.

In another aspect, the present disclosure provides a method of decreasing expression of an aberrant FMR1 gene product in a cell, comprising contacting the cell with an agent under conditions whereby the agent is introduced into the cell, thereby decreasing expression of the aberrant FMR1 gene product in the cell.

In another aspect, the present disclosure provides a method of modulating FMR1 splicing and/or expression in a cell, comprising contacting the cell with an agent (e.g., a polynucleotide) under conditions whereby the agent is introduced into the cell, thereby modulating FMR1 splicing and/or expression in the cell.

In another aspect, the present disclosure provides a method of increasing the level of FMRP in a cell, comprising contacting the cell with an agent (e.g., a polynucleotide) under conditions whereby the agent is introduced into the cell, such that the level of FMRP in the cell is enhanced.

In another aspect, the present disclosure provides a method of enhancing the level of FMRP in a cell, comprising contacting the cell with an oligonucleotide which is complementary to at least 8 contiguous nucleotides of a sequence set forth in SEQ ID NOs:24-42, such that the level of FMRP in the cell is enhanced.

In another aspect, the present disclosure provides a method of reducing CGG triplet repeat expansion in FMR1 5′ UTR in a cell, comprising contacting the cell with an agent that reduces expression of an aberrant FMR1 gene product under conditions whereby the agent is introduced into the cell, thereby reducing CGG triplet repeat expansion in the cell.

In some embodiments, the fragile X-associated disorder is FXS.

In some embodiments, the aberrant FMR1 gene product comprises FMR1-217.

In some embodiments, the agent is a polynucleotide (e.g., any one of the modified polynucleotides disclosed herein).

In some embodiments, the method increases expression of fragile X messenger ribonucleoprotein (FMRP) in the subject.

In another aspect, the present disclosure provides an agent that decreases expression of an aberrant FMR1 gene product.

In another aspect, the present disclosure provides an agent that modulates splicing and/or expression of an FMR1 gene (e.g., decreasing splicing between Exons 1 and 2 of FMR1-217 or decreasing a protein encoded by FMR1-217).

In yet another aspect, the present disclosure provides a pharmaceutical composition, comprising any one or more of the agents disclosed herein, and one or more pharmaceutically acceptable excipients, diluents, or carriers.

In some embodiments, the agent is a polynucleotide (e.g., any one of the modified polynucleotides disclosed herein).

In another aspect, the present disclosure provides an antisense oligonucleotide (ASO), wherein the ASO specifically binds a contiguous nucleotide sequence set forth in any one of SEQ ID NOs:24-42, and wherein the contiguous nucleotide sequence is at least 12 nucleotides in length.

In another aspect, the present disclosure provides a pharmaceutical composition, comprising at least one ASO disclosed herein and a pharmaceutically acceptable excipient, diluent, and/or carrier.

In another aspect, the present disclosure provides a method of treating a disease in a subject in need thereof, comprising administering to the subject a therapeutically effective amount of any one of the pharmaceutical compositions disclosed herein. In some embodiments, the disease is fragile X syndrome (FXS).

In another aspect, the present disclosure provides a method of reducing a FMR1-217 transcript in a cell, comprising contacting the cell with an effective amount of the at least one ASO disclosed herein or any one of the pharmaceutical compositions disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows a genome browser view of FMR1 RNA in 7 typically developing (“TD” or “control”) and 10 fragile X syndrome (FXS) patients sequenced from white blood cells (WBCs).

FIG. 2 shows a genome browser view of exon 1 and intron 1 of FMR1 RNA in 7 typically developing individuals and 10 fragile X syndrome patients sequenced from white blood cells.

FIG. 3 illustrates a non-limiting approach, using antisense oligonucleotides (ASOs), for blocking isoform 12 production, increasing isoform 1 production, and increasing FMRP levels.

FIG. 4 shows a schematic of FMR1 iso1 and iso12 pre-mRNAs. The numbered boxes (704-714) refer to antisense oligonucleotides complementary to regions in intron 1, that span intron 1 and iso12 junction, and within iso12. Iso1_1 F, Iso1_1 R, Exon1 F, Exon1 R, and Iso12_1 R refer to primers (F, forward; R, reverse) that were used to detect RNA levels by RT-qPCR.

FIG. 5 shows RT-qPCR data demonstrating a reduction in iso12 and increase in iso1. The asterisk refers to p<0.05.

FIGS. 6A-6B show RT-qPCR data from a fully methylated FXS cell line (FXS1, GM07365). FIG. 6A shows an increase in FMR1 iso12 upon 5-AzaC treatment and a partial rescue of the FMR1 iso12 increase when combined with the ASO treatment. FIG. 6B shows an increase in FMR1 iso1 upon 5-AzaC treatment and a further increase when combined with the ASO treatment. The asterisks refer to p<0.05.

FIGS. 7A-7B show FMRP levels. FIG. 7A shows western blot data for an FXS1 LCL cell line in duplicates (the upper panel), demonstrating an increase in FMRP after treatment with 1 μM 5-AzaC and ASO treatment (80 nM of both antisense oligonucleotides 713 and 714) when compared to DMSO or 5-AzaC only treated samples. The mouse brains (hippocampus tissue) from a wild-type mouse and an Fmr1 knock-out mouse were loaded as controls. The bottom panel represents GAPDH protein levels used to normalize the protein amounts loaded in each sample. FIG. 7B shows quantification of the FMRP protein levels relative to GAPDH protein levels as seen on the western blot in FIG. 7A.

FIGS. 8A-8C show FMR1 iso1 and iso12 levels in fibroblast cells from six individuals. FIG. 8A is a table showing the number of CGG repeats in the FMR1 RNA 5′ UTR from three healthy males and three premutation carrier males for FXS. FIG. 8B shows RT-qPCR data of FMR1 iso1 levels in fibroblast cells from the six individuals, normalized to GAPDH RNA levels. FIG. 8C compares the FMR1 iso12 level in individual P1 to those in the other premutation carriers and healthy control samples.

FIGS. 9A-9C A truncated isoform of FMR1 mRNA identified in a subset of FXS individuals. FIG. 9A Integrative Genomics Viewer (IGV) tracks of RNA-seq data for FXS and TD individuals for the FMR1 gene. FMR1 RNA was detected in all TD individuals, and FXS individuals 1-21. The thick-lined box marked on the FMR1 gene illustrated at the bottom shows the region of intron 1 with differential reads between TD (1-13) and FXS (1-21) individuals.

FIG. 9B Expanded view map to an exon that comprises the annotated FMR1-217 isoform. All annotated FMR1 isoforms and sequence data for FMR1-217 PCR fragments from FXS RNA sample are shown in Table 3 and FIGS. 9E-9H. H refers to high and L refers to low FMR1. FIG. 9C. The full length FMR1 RNA (exons-grey boxes) and the FMR1-217 isoform (exons-grey boxes) are illustrated with the CGG repeats in the 5′UTR (UTRs-black boxes). The proportion of full length FMR1 to FMR1-217 was quantified by RT-qPCR in TD, H FMR1 (N=7), and L FMR1 (N=5) individuals. The forward (F) and reverse (R) primers used for q-PCR are shown. The total FMR1 RNA relative to GAPDH RNA levels was significantly reduced in H FMR1 and L FMR1 vs TD (*P<0.05, t test). Bar graphs indicate mean, and error bars indicate±SEM. FIG. 9D Summary table of changes in alternative splicing events from L FMR1 vs H FMR1 samples detected by replicate multivariate analysis of transcript splicing (rMATS; see Shen et al., rMATS: Robust and flexible detection of differential alternative splicing from replicate RNA-Seq data, Proc. Natl. Acad. Sci. 111(51):E5593-5601 (2014)) at an FDR<5% and a difference in the exon inclusion levels (PSI, Percent spliced-in) between the genotypes (deltaPSI) of ≥5%. Schematic for the splicing event categories is shown at the left of the table. FIG. 9E FMR1-217 isoform was identified in RNA samples generated from leukocytes (individual FXS-05). DNase-treated RNA samples were reverse transcribed using an oligo(dT)₂₀ primer, and the PCR product, generated using primers Ex1F and 217R, was sequenced. FIG. 9F The predicted protein product of the FMR1-217 isoform. The predicted protein length is 31 amino acids, with a mass of 3,524 Da. FIG. 9G Alignment of the sequencing data of the PCR product using primers Ex1F and 217R to FMR1 gene is displayed. The poly(A) site was identified by sequencing the PCR product of primer 217F and oligo(dT)₂₀ primer. FIG. 9H FMR1 isoforms annotated in the GRCh38.p13 genome assembly. The FMR1-217 isoform (ENST00000621447.1) is marked with a thick-lined box.

FIG. 10 Correlation of FXS molecular parameters with intelligence quotient (IQ). Three-dimensional comparison of indicated parameters. The inset shows samples with 100% methylation. The increasing size of the dots represent increase in FMRP levels, and the darkness from low to high represent increase in IQ levels (see Table 4).

FIGS. 11A-11E FMR1-217 is derived from FMR1, requires the CGG expansion, and is expressed in human postmortem brain tissues (FXS and premutation carriers), and in skin-derived fibroblasts (premutation carrier). FIG. 11A Integrative Genomics Viewer (IGV) tracks of RNA-seq data (Tran et al., Widespread RNA editing dysregulation in brains from autistic individuals, Nat. Neurosci. 22(1):25-36 (2019)) for FXS and TD individuals for the FMR1 gene. FIG. 11B IGV tracks of selected regions of FMR1 re-analyzed from the RNA-seq data of Vershkov et al. (FMR1 Reactivating Treatments in Fragile X iPSC-Derived Neural Progenitors In Vitro and In Vivo, Cell Rep. 26:2531-39 (2019)), who deleted the FMR1 CGG expansion by CRISPR/Cas9 gene editing. Biologic duplicate of iPSC-derived neural stem cells (NSCs) from FXS individuals (FXS-NSC) treated with vehicle or 5-aza-2-deoxycytidine (5-azadC) as well as isogenic CGG-edited samples are shown. FMR1-217 reads are detected only in the 5-azadC-treated samples. FIG. 11C IGV tracks of selected regions of FMR1 re-analyzed from the RNA-seq data of Liu et al. (Rescue of Fragile X Syndrome Neurons by DNA Methylation Editing of the FMR1 Gene, Cell 172:979-91 (2018)), who performed targeted FMR1 gene demethylation in FXS iPSCs and iPSC-derived neurons. iPSCs derived from FXS individuals were incubated with viruses expressing a mock guide RNA (i_mock), or an FMR1 guide RNA and catalytically inactive Cas9 fused to the Tet1 demethylase (i_Tet1). iPSC-derived neurons from to FXS individuals were treated with a mock guide RNA (N1 mock, N2 mock), or an FMR1 guide RNA and catalytically inactive Cas9 fused to the Tet1 demethylase (N1_Tet1, N2_Tet1, N3_Tet1). All cells were incubated with an FMR1 guide RNA and catalytically inactive Cas9 fused to the Tet1 demethylase express FMR1-217. FIG. 11D Experimental design for RNA extraction from post-mortem cortical tissue obtained from 6 FXS males (F1-F6) and 5 typically developing (T1-T5) age-matched males. RT-qPCR data for cortical tissue-derived RNA samples representing abundance for FMR1 and FMR1-217 isoforms relative to GAPDH RNA. Each sample was analyzed in duplicate. Primers used for amplification are represented in FIG. 9C (**P<0.01, t test). FIG. 11E Schematic diagram of fibroblast generated from skin biopsies obtained from three male premutation carriers (P1-P3) and three male TD individuals (T1-T3). The table shows patient de-identified designation, genotypes, and CGG repeat numbers in the 5′UTR in the FMR1 gene. ND: not determined. qPCR data for fibroblast-derived RNA samples representing abundance for FMR1 and FMR1-217 isoforms relative to GAPDH RNA. Each sample was analyzed in duplicate. Primers used for amplification are represented in FIG. 9C.

FIGS. 12A-12G FMR1-217 is expressed in lymphoblast cell cultures from FXS individuals. FIG. 12A Sample information for lymphoblast cell lines (LCLs) (Coriell Institute, NJ) from two FXS and two TD members of a family. FMRP and GAPDH (loading control) levels were determined by western blots. Ratios of FMRP/GAPDH normalized to FXS1 are shown below the blot. FMRP quantification by LUMINEX® Microplex immunochemistry assay are shown in ng FMRP/μg total protein). FIG. 12B The proportion of full length FMR1 to FMR1-217 was quantified using RT-qPCR in the TD and FXS2 LCLs relative to GAPDH RNA levels. Primers used for q-PCR are shown in the gene illustration. The total FMR1 RNA was unchanged but the proportion of FMR1-217 was significantly higher in FXS2 LCL compared to TD LCL. FIG. 12C Schematic diagram of the 5-AzadC treatment (1 μM for 7 days) of the FXS1 and FXS2 LCLs to determine FMR1 isoforms and FMRP levels after demethylation. DMSO treated cells were used as a vehicle control. FIGS. 12D-12E The proportion of full length FMR1 to FMR1-217 was quantified using RT-qPCR in the FXS1 and FXS2 LCLs treated with 5-AzadC relative to vehicle, normalized to GAPDH RNA levels (**P<0.001, t test). FIGS. 12F-12G FMRP levels were determined using western blots relative to GAPDH in FXS1 and FXS2 LCLs treated with DMSO or 5-AzadC (See FIG. 13A) Ratios of FMRP/GAPDH are shown for FXS1 and FXS2 cells, respectively. Histograms indicate mean values (N=2); error bars indicate SEM.

FIG. 13A Western blots showing FXS1 and FXS2 cells respectively treated with DMSO or 5-AzadC (as treated in FIG. 12C and quantified in FIGS. 12F-12G). FIG. 13B The decrease in MALAT1 RNA levels relative to GAPDH RNA was quantified by RT-qPCR in the TD1 LCL treated with MALAT1 ASO (80 nM and 100 nM) for 48 hours. Untreated cells were used as negative controls (* represents P<0.05, t test). The right panel shows a decrease in MALAT1 RNA levels compared to GAPDH RNA levels, quantified using RT-qPCR in the TD1 LCL treated with 80 nM MALAT1 gapmer ASO for 48 hours or 72 hours. Untreated cells were used as negative controls (* represents P<0.05 using t test). FIG. 13C FXS2 LCLs were treated with either 80 nM of ASOs 704 and 705, 709 and 710 or 713 and 714 for 72 hours. RNA levels for FMR1-217 and FMR1 full length RNA were quantified by RT-qPCR using primers as in FIG. 9C. ASOs 713 and 714 reduced FMR1-217 levels whereas FMR1 full length RNA levels were increased (* represents P<0.05, t test). FIG. 13D FXS2 LCLs were treated with either 80 nM or 160 nM of ASOs 713 and 714 or 80 nM of Malat1 gapmer ASO for 72 hours. RNA levels for FMR1-217 and FMR1 full length RNA were quantified by RT-qPCR using primers as in FIG. 9C. ASOs 713 and 714 reduced FMR1-217 levels at both 80 nM and 160 nM concentrations whereas FMR1 full length RNA levels were increased. No change in the FMR1 isoform levels was observed upon MALAT1 ASO treatment (* represents P<0.05, t test).

FIGS. 14A-14F ASOs targeting FMR1-217 restore FMRP levels in FXS LCLs with partial or complete FMR1 gene methylation. FIG. 14A ASOs designed against the FMR1-217 RNA are illustrated. (Intron specific: 704-706, intron-exon junction specific: 707-710 and exon specific: 711-714). FIG. 14B Schematic diagram of the ASO treatment (80 nM for 72 hours) of the FXS2 LCLs to determine FMR1 isoform and FMRP levels after demethylation. DMSO treated cells were used as a vehicle control (****P<0.0001, **P<0.01, t test). FIG. 14C FMRP levels were determined for FXS2 LCLs treated with DMSO (vehicle) and ASOs as described in FIG. 12A. TD LCLs were also probed for FMRP on the same western blots. Ratios of FMRP/GAPDH normalized to FXS1 are shown below the blot. FIG. 14D Fully methylated FXS1 LCLs were treated with ASOs 713 and 714 (80 nM each) followed by 5-AzadC (1 uM) added on consecutive days 2-9 after which RNA and protein were extracted. FMR1-217 and FMR1 isoforms were assessed using qPCR primers as shown in FIG. 9C was determined using one way ANOVA with multiple comparisons test (****P<0.0001, ***P<0.001, **P<0.01, *P<0.05). Data information: bar graphs indicate mean, error bars indicate±SEM. FIG. 14E Western blot of FMRP and GAPDH from FXS1 LCLs treated with DMSO, 5-AzadC, or 5-AzadC plus ASOs as in FIG. 13A. FIG. 14F Histogram depicting quantification of western blot for FXS1 cells treated with DMSO, 5-AzadC and ASO or 5-AzadC alone (N=3). Significance was determined using one way ANOVA with multiple comparisons test ((****P<0.0001, Data information: bar graphs indicate mean, error bars indicate±SEM.

FIGS. 15A-15F ASOs targeting FMR1-217 restore FMRP levels in FXS fibroblasts with an inactive FMR1 gene treated with 5-AzadC. FIG. 15A Dermal fibroblasts derived from a FXS individual (GM05131B, Coriell Institute) were cultured with 5-AzadC for 8 days and then treated with ASOs 713/714 (100 nM each) for 72 hours prior to RNA and protein extraction. FIG. 15B RT-qPCR analysis of FMR1-217, FMR1, and GAPDH RNAs in dermal fibroblasts treated with DMSO, ASOs 713/714, 5-AzadC, or the ASOs 713/714 plus 5-AzadC. The amounts of FMR1-217 and FMR1 were made relative to GAPDH. (*P<0.05, **P<0.01, one way ANOVA with multiple comparisons test). FIG. 15C Western blots of FMRP and GAPDH from the dermal fibroblasts treated as in FIG. 15B. Quantification of FMRP relative to GAPDH is noted at right. *p<0.05. Histogram depicting quantification of western blot (N=3). Significance was determined using one way ANOVA with multiple comparisons test (*p<0.05, one way ANOVA with multiple comparisons test). Data information: bar graphs indicate mean, error bars indicate±SEM. FIG. 15D Lung fibroblasts derived from a FXS individual (GM07072, Coriell Institute) were cultured with 5-AzadC for 8 days and then treated with ASOs 713/714 (100 nM each) for 72 hours prior to RNA and protein extraction. RT-qPCR analysis of FMR1-217, FMR1, and GAPDH RNAs in lung fibroblasts treated with DMSO, ASOs 713/714, 5-AzadC, or the ASOs 713/714 plus 5-AzadC. The amounts of FMR1-217 and FMR1 were made relative to GAPDH. (*p<0.05, **p<0.01, ***p<0.001, one way ANOVA with multiple comparisons test). FIG. 15E Western blots of FMRP and GAPDH from the lung fibroblasts treated as in FIG. 15B. Quantification of FMRP relative to GAPDH is noted below (N=2). Significance was determined using one way ANOVA with multiple comparisons test (P<0.0001****, P<0.001, ***P<0.01**, one way ANOVA with multiple comparisons test). Data information: bar graphs indicate mean, error bars indicate±SEM. FIG. 15F Model depicting active FMR1 transcription in FXS cells (or after treatment with demethylating agents to activate FMR1 transcription) result in production of mis-spliced FMR1-217. Down-regulation of FMR1-217 with an ASO results in rescue of correctly spliced FMR1 transcripts and restoration of FMRP protein.

FIG. 16A Additional ASO sequences. FIG. 16B 72-hour treatment with 160 nM of each ASO in lymphoblastoid cell line FXS2. Total RNA was extracted using TRIZOL® Reagent (Thermo Fisher Scientific #15596026). One μg of total RNA was primed with oligo(dT)20 to generate cDNA with a QuantiTect cDNA synthesis kit using random hexamers (FIG. 9E). qPCR was performed using the iTaq™ Universal SYBR® Green Supermix on a QuantStudio 3 qPCR machine in triplicate. The fold change of full length FMR1 and FMR1-217 in ASO treated cells relative to vehicle (control) was measured using qPCR. The RNA levels were normalized to GAPDH RNA (*P values measured using t test).

FIGS. 17A-17G. Gene expression changes in leukocytes derived from FXS individuals and identification of a truncated FMR1 RNA transcript. FIG. 17A Schematic diagram of leukocyte isolation from fresh blood samples from FXS male (N=29) and age-matched TD male (N=13) individuals and subsequent RNA-seq. The data were analyzed for changes in differential gene expression (DGE) and differential alternative splicing (DAS).

FIG. 17B Summary table for changes in alternative splicing events in FXS vs. TD leukocytes detected by rMATS (Shen et al., rMATS: Robust and flexible detection of differential alternative splicing from replicate RNA-Seq data, Proc. Natl. Acad. Sci. 111(51):E5593-5601 (2014)) at a false discovery rate (FDR)<5% and a difference in the exon inclusion levels (PSI, Percent spliced-in) between the genotypes (deltaPSI) of ≥5%. Schematic for the splicing event categories is shown at the left of the table (see also Tables 13-19 and FIG. 22A). FIG. 17C Violin plots of alternative splicing in FXS vs. TD leukocytes indicating PSI for each type of event. FIG. 17D RT-PCR showing exon 3 skipping of the LAIR2 RNA in TD (N=3) and FXS (N=9) samples. FIG. 17E Normalized gene counts (transcripts per million, TPM) obtained from RNA-seq data analysis for total FMR1 (all isoforms), FMR1-205 (encodes full-length 632 amino acid FMRP), FMR1-217 (a mis-spliced RNA, see FIG. 17G), and FXR2, a paralog of FMR1. The color scale from red to green denotes highest to lowest gene counts. FIG. 17F IGV viewer tracks of RNA-seq data for FXS and TD individuals for the FMR1 gene. FMR1 RNA is detected in all TD individuals (blue) and FXS individuals 1-21 (coral). The black box marked on the FMR1 gene illustrated at the bottom shows the region of intron 1 with differential reads between TD (1-13) and FXS (1-21) individuals. Expanded view of this region is shown in the bottom. FIG. 17G An expanded view of the FMR1 RNA marked with a black box in FIG. 17G. The reads map to an exon that comprises the annotated FMR1-217 isoform. “H” refers to high and “L” refers to low FMR1. Sequence data for FMR1-217 PCR fragments from FXS RNA sample are shown in FIG. 22G.

FIGS. 18A-18E. Correlation of FXS molecular parameters with IQ. FIG. 18A FMR1 gene methylation (in percent as determined by methylation (MPCR) PCR analysis), FMRP levels (ng/μg total protein), CGG repeat number, FMR1 (all isoforms, TPM), FMR1-217 (TPM), and full-length FMR1-205 (TPM) in leukocytes are listed as well as IQ (Stanford-Binet) for each FXS individual. N/A, not available (see also Tables 9-12 and FIGS. 26A-27B). Color shading of cells corresponds to levels of each parameter (green to red represent increasing level). FIG. 18B Correlation coefficients for pairwise comparisons for each parameter (less CGG expansion and methylation) listed in FIG. 18A. Correlations were based on parameters from 19 FXS individuals because some data were not available from all the 29 samples. FIG. 18C 3-dimensional comparison of all parameters (less CGG expansion and methylation) listed in FIG. 18A. The color from green to red represents increasing FMR1-205 levels (see also FIG. 18A). The illustration is based on parameters from 19 FXS individuals because some data were not available from all the 29 samples. FIG. 18D FMR1-217 RNA levels (TPM) were assessed between FXS samples with CGG repeat number mosaicism (N=8) or without (full expansion only) (N=21). FIG. 18E FMR1-217 RNA levels (TPM) were assessed between FXS samples with methylation mosaicism (N=7) or without (full expansion only) (N=13) (*P<0.05, t test).

FIGS. 19A-19H. FMR1-217 is derived from FMR1, requires the CGG expansion, and is expressed in human postmortem brain tissues (FXS and premutation carriers) and in skin-derived fibroblasts (premutation carrier). FIG. 19A Sample information for postmortem FXS frontal cortex, premutation FXS carriers, and TD individuals (derived from Tran et al., Widespread RNA editing dysregulation in brains from autistic individuals, Nat. Neurosci. 22(1):25-36 (2019)). RNA-seq datasets GSE107867 (NIH samples) and GSE117776 were reanalyzed for differential gene expression (DGE) and differential alternative splicing (DAS). The TPM for FMR1 RNA in the samples is shown. FIG. 19B IGV tracks of RNA-seq data (Tran et al., Nat. Neurosci. 22(1):25-36 (2019)) for FXS and TD individuals for the FMR1 gene. FIG. 19C IGV tracks of selected regions of FMR1 reanalyzed from the RNA-seq data of Vershkov et al. FMR1 Reactivating Treatments in Fragile X iPSC-Derived Neural Progenitors In Vitro and In Vivo, Cell Rep. 26(10):2531-39 (2019), who deleted the FMR1 CGG expansion by CRISPR/Cas9 gene editing. Biologic duplicate of iPSC-derived neural stem cells (NSCs) from FXS individuals (FXS-NSC) treated with vehicle or 5-AzadC and isogenic CGG-edited samples are shown. FMR1-217 reads (coral) are detected only in the 5-AzadC-treated samples. FIG. 19D Total FMR1, full-length FMR1-205, and FMR1-217 reads (TPM) of the samples noted in panel C are listed. FIG. 19E IGV tracks of selected regions of FMR1 reanalyzed from the RNA-seq data of Liu et al. Rescue of Fragile X syndrome neurons by DNA methylation editing of the FMR1 gene, Cell 172:979-91 (2018)), who performed targeted FMR1 gene demethylation in FXS iPSCs and iPSC-derived neurons. FXS iPSCs/iPSC-derived neurons incubated a mock guide RNA (blue tracks) (i_mock or N1_mock, N2_mock) or an FMR1 guide RNA (coral tracks) and Cas9 fused to the Tet1 demethylase (i_Tet1 or N1_Tet1, N2_Tet1, N3_Tet1). FIG. 19F Total FMR1, full-length FMR1-205, and FMR1-217 reads (TPM) of the samples noted in panel E are listed. FIG. 19G Experimental design of RNA extraction from postmortem cortical tissue obtained from six FXS males (F1 to F6) and five TD (T1 to T5) age-matched males. RT-qPCR data for cortical tissue-derived RNA samples representing abundance for FMR1 and FMR1-217 isoforms relative to GAPDH RNA. Each sample was analyzed in duplicate. Primers used for amplification are represented in FIG. 22F (**P<0.01, t test). FIG. 19H Schematic diagram of fibroblast generated from skin biopsies obtained from three male premutation carriers (P1 to P3) and three male TD individuals (T1 to T3). The table shows patient deidentified designation, genotypes, and CGG repeat numbers in the 5′UTR in the FMR1 gene. ND, not determined. qPCR data for fibroblast-derived RNA samples representing abundance for FMR1 and FMR1-217 isoforms relative to GAPDH RNA are shown. Each sample was analyzed in duplicate. Primers used for amplification are represented in FIG. 22F and Tables 9-12.

FIGS. 20A-20F. Antisense oligonucleotides (ASOs) targeting FMR1-217 restored FMRP levels in FXS2 LCLs with incomplete methylation. FIG. 20A Sample information for lymphoblast cell lines (LCLs) (Coriell Institute, NJ) from two FXS and two TD members of a family is shown. FMRP quantification by LUMINEX® Microplex immunochemistry assay is shown in ng FMRP/μg total protein. The western blot below the table shows FMRP and GAPDH (loading control) determinations in the above noted LCLs. Ratios of FMRP/GAPDH normalized to FXS1 are shown below the blot. FIG. 20B The proportion of full-length FMR1 to FMR1-217 was quantified using RT-qPCR in the TD and FXS2 LCLs relative to GAPDH RNA levels. Primers used for qPCR are shown in the gene illustration. The total FMR1 RNA was unchanged, but the proportion of FMR1-217 was significantly higher in FXS2 LCL compared to TD LCL. FIG. 20C ASOs complementary to FMR1-217 RNA are illustrated (intron specific: 704 to 706, intron-exon junction specific: 707 to 710 and exon specific: 711 to 714). FIG. 20D Schematic diagram of the ASO treatment (80 nM for 72 hours) of the FXS2 LCLs to determine FMR1 isoform and FMRP levels after demethylation. DMSO-treated cells were used as a vehicle control (****P<0.0001, **P<0.01, t test). FIG. 20E FMRP levels were determined for FXS2 LCLs treated with DMSO (vehicle) and ASOs as described in FIG. 21A. TD LCLs were also probed for FMRP on the same western blots. Ratios of FMRP/GAPDH normalized to FXS1 are shown below the blot. FIG. 20F Model depicting that ASO-mediated decrease of mis-spliced FMR1-217 in FXS2 LCLs can restore FMR1 full-length RNA and consequently FMRP levels.

FIGS. 21A-21F. ASOs targeting FMR1-217 in combination with 5-AzadC restored FMRP levels in FXS cells with complete FMR1 gene methylation. FIG. 21A Schematic diagram of the fully methylated FXS cells treated with ASOs 713 and 714 (80 nM each) followed by 5-AzadC (1 μM) added on consecutive days 2 to 9 after which RNA and protein were extracted. FIG. 21B FMR1-217 and FMR1 isoforms were assessed using qPCR primers as shown in FIG. 22F and were analyzed using one-way ANOVA with multiple comparisons test (****P<0.0001, ***P<0.001, **P<0.01, *P<0.05). Data information: bar graphs indicate mean, and error bars indicate±standard error of the mean (SEM). FIG. 21C Western blot of FMRP and GAPDH from FXS1 LCLs treated with DMSO, 5-AzadC, or 5-AzadC plus ASOs. Histogram depicting quantification of western blot for FXS1 cells treated with DMSO, 5-AzadC, and ASO or 5-AzadC alone (N=3) in arbitrary units. Significance was determined using one-way ANOVA with multiple comparisons test (****P<0.0001). Data information: bar graphs indicate mean, and error bars indicate±SEM. FIG. 21D Lung fibroblasts derived from an FXS individual (GM07072, Coriell Institute) were cultured with 5-AzadC for 8 days and then treated with ASOs 713/714 (100 nM each) for 72 hours prior to RNA and protein extraction. RT-qPCR analysis of FMR1-217, FMR1, and GAPDH RNAs in lung fibroblasts treated with DMSO, ASOs 713/714, 5-AzadC, or the ASOs 713/714 plus 5-AzadC. The amounts of FMR1-217 and FMR1 were made relative to GAPDH (*P<0.05, **P<0.01, ***P<0.001, one-way ANOVA with multiple comparisons test). FIG. 21E Western blots of FMRP and GAPDH from the lung fibroblasts treated as in panel B. Quantification of FMRP relative to GAPDH is noted below (N=2) in arbitrary units. Significance was determined using one-way ANOVA with multiple comparisons test (****P<0.0001, ***P<0.001, **P<0.01, one-way ANOVA with multiple comparisons test). Data information: bar graphs indicate mean, and error bars indicate±SEM. FIG. 21F Model depicting active FMR1 transcription in FXS cells (or after treatment with demethylating agents to activate FMR1 transcription) results in the production of mis-spliced FMR1-217. Downregulation of FMR1-217 with an ASO results in rescue of correctly spliced FMR1 transcripts and restoration of FMRP.

FIGS. 22A-22G. FIG. 22A Volcano plot of log 2 of fold change (log 2FC) of RNA levels (FXS vs TD). Statistically significant changes (P value<0.0002) are shown as blue dots (down-regulated) and red dots (up-regulated). Gray dots refer to unchanged RNAs. See also Tables 13-19. FIG. 22B Histograms for TPM values for RNAs that were up or downregulated in FXS vs TD. *p<0.05; **p<0.01). FIG. 22C Histograms of RT-qPCR validations of up or down-regulated RNAs in FXS (N=7) and TD (N=5) leukocytes. See also FIG. 22B. FIG. 22D Metagene profiles using deepTools 2 for distribution of H3K4me3 marks along gene lengths. A similar increase in ChIP signal irrespective of genotype is seen at the transcription start site (TSS) for the H3K4me3 IP. FXS (N=2) and TD (N=3). FIG. 22E Metagene profiles using deepTools 2 for distribution of H3K36me3 marks along gene lengths. A similar increase in ChIP signal irrespective of genotype is seen in the gene body and transcription end site (TES) for H3K36me3 ChIP. FXS (N=2) and TD (N=3). FIG. 22F The full length FMR1 RNA (exons-blue boxes) and the FMR1-217 isoform (exons-orange boxes) are illustrated with the CGG repeats in the 5′UTR (UTRs-black boxes). The proportion of full length FMR1 to FMR1-217 is quantified by RT-qPCR in TD; H FMR1 (N=7) and L FMR1 (N=5) individuals. The forward (F) and reverse (R) primers used for q-PCR are shown. The total FMR1 RNA relative to GAPDH RNA levels was significantly reduced in H FMR1 and L FMR1 vs TD (*P<0.05, t test). Bar graphs indicate mean, error bars indicate±SEM. FIG. 22G FMR1-217 isoform was identified in RNA samples generated from leukocytes (individual FXS-05). DNase-treated RNA samples were reverse transcribed using an oligo(dT)₂₀ and the PCR product generated using primers Ex1F and 217R was sequenced. Alignment of the sequencing data of the PCR product using primers Ex1F and 217R to FMR1 gene is displayed. The poly(A) site was identified by sequencing the PCR product of primer 217F and oligo(dT)₂₀ primer. The predicted protein product of the FMR1-217 isoform is shown.

FIGS. 23A-23C. FIG. 23A IQ comparison between FXS individuals with (N=8) or without (N=18) CGG repeat number mosaicism (full expansion only) (left) or with (N=6) or without (N=12) methylation mosaicism. Related to FIG. 18A and Tables 9-12 and FIGS. 26A-27B. FIG. 23B FMRP level comparison between FXS samples with (N=5) or without (N=15) CGG repeat number mosaicism (full expansion only). In the right panel, FMRP level comparison between FXS samples with (N-5) or without (N=11) methylation mosaicism (full expansion only) (N=11). (*P<0.05, **P<0.01, t test). Related to FIG. 18A and Tables 9-12 and FIGS. 26A-27B. FIG. 23C FMR1-205 level comparison between FXS samples with (N=8) or without (N=21) CGG repeat number mosaicism (full expansion only). In the right panel, FMR1-205 level comparison levels between FXS samples with (N=7) or without (N=13) methylation mosaicism (full expansion only). (*P<0.05, **P<0.01, t test). Related to FIG. 18A, Tables 9-12, and FIGS. 26A-27B.

FIGS. 24A-24D. FIG. 24A Volcano plot of log 2FC of RNA level change (DGE) comparing FXS (N=2) and TD (N=2) postmortem brain. Statistically significant changes (Padj value<0.05) are shown as blue dots (down-regulated) and red dots (up-regulated) compared to unchanged RNAs (gray dots). (See also Tables 20-28). FIG. 24B Changes in alternative splicing comparing TD vs FXS postmortem brain detected by rMATS (Shen et al., rMATS: Robust and flexible detection of differential alternative splicing from replicate RNA-Seq data, Proc. Natl. Acad. Sci. 111(51):E5593-5601 (2014)) at an FDR<5% and a difference in the exon inclusion levels (PSI, Percent spliced-in) between the genotypes (deltaPSI) of ≥5%. Schematic for the splicing event categories is shown at the left of the table (See also Tables 20-28). FIG. 24C Volcano plot of log 2FC of RNA level change (FXS vs pre-mutation carriers (NIH), N=2 per genotype). Statistically significant changes (Padj value<0.05) are shown as blue dots (down-regulated) and red dots (up-regulated) compared to unchanged RNAs (gray dots). (See also Tables 20-28). FIG. 24D Changes in alternative splicing comparing FXS vs pre-mutation carriers (NIH), detected by rMATS (Shen et al., Proc Natl Acad Sci USA. 111(51):E5593-601 (2014)) at an FDR<5% and a difference in the exon inclusion levels (PSI, Percent spliced-in) between the genotypes (deltaPSI) of ≥5%. Schematic for the splicing event categories is shown at the left of the table (See also Tables 20-28).

FIGS. 25A-25F. FIG. 25A The decrease in MALAT1 RNA levels relative to GAPDH RNA was quantified by RT-qPCR in the TD1 LCL treated with MALAT1 ASO (80 nM and 100 nM) for 48 hours. Untreated cells were used as negative controls (* represents P<0.05, t test). The right panel shows a decrease in MALAT1 RNA levels compared to GAPDH RNA levels, quantified using RT-qPCR in the TD1 LCL treated with 80 nM MALAT1 gapmer ASO for 48 hours or 72 hours. Untreated cells were used as negative controls (* represents P<0.05 using t test). FIG. 25B FXS2 LCLs were treated with either 80 nM or 160 nM of ASOs 713 and 714 or 80 nM of Malat1 gapmer ASO for 72 hours. RNA levels for FMR1-217 and FMR1 full length RNA were quantified by RT-qPCR using primers as in FIG. 18C. ASOs 713 and 714 reduced FMR1-217 levels at both 80 nM and 160 nM concentrations whereas FMR1 full length RNA levels were increased. No change in the FMR1 isoform levels was observed upon MALAT1 ASO treatment (* represents P<0.05, t test). FIG. 25C FXS2 LCLs were treated with either 80 nM of ASOs 704 and 705, 709 and 710 or 713 and 714 for 72 hours. RNA levels for FMR1-217 and FMR1 full length RNA were quantified by RT-qPCR using primers as in FIG. 18C. ASOs 713 and 714 reduced FMR1-217 levels whereas FMR1 full length RNA levels were increased (* represents P<0.05, t test). FIG. 25D Western blots of FMRP and GAPDH from the FXS1 LCL cells treated with 5-AzadC or 5-AzadC and ASOs for 1 week prior to collection (scheme as in FIG. 21A). Quantification of FMRP relative to GAPDH in arbitrary units is noted below. Data information: bar graphs indicate mean, error bars indicate±SEM. FIG. 25E RT-qPCR analysis of FMR1-217, FMR1, and GAPDH RNAs in dermal fibroblasts (GM05131B, Coriell Institute) treated with DMSO, ASOs 713/714, 5-AzadC, or the ASOs 713/714 plus 5-AzadC. The amounts of FMR1-217 and FMR1 were made relative to GAPDH. (*P<0.05, **P<0.01, one way ANOVA with multiple comparisons test). FIG. 25F Western blots of FMRP and GAPDH from the dermal fibroblasts treated as in FIG. 25B. Quantification of FMRP relative to GAPDH in arbitrary units is noted at right. *p<0.05. Histogram depicting quantification of western blot (N=3). Significance was determined using one way ANOVA with multiple comparisons test (*p<0.05, one way ANOVA with multiple comparisons test). Data information: bar graphs indicate mean, error bars indicate±SEM.

FIGS. 26A-26C. FMR1 Transcript Levels.

FIGS. 27A-27B. FXS Sample Details.

FIGS. 28A-28B. FIG. 28A is a schematic diagram of ASOs that are complementary to a pseudo exon of FMR1 (FMR1 217 exon 2). ASOs 713 and 714 are indicated. FIG. 28B The sequences of ASOs. The lower case “e” before each base indicates that the base contains a 2′-O-methoxyethyl ribose sugar. The uppercase letters indicate the sequence of nucleobases in each ASO. The backbone of these oligonucleotides is either fully modified with phosphorothioate linkages (ASOs 2826, 2828, 2829, 2831, 2832, 2833, and 2834) or is a combination of phosphorothioate and phosphodiester linkages (ASOs 2827 and 2830). The positions of the phosphorothioate linkages are indicated by a hashtag between the parentheses defining each nucleotide, “)#(”, while phosphodiester linkages are indicated by no symbol between the parentheses,“)(”.

FIGS. 29A-29B. FIG. 29A is a schematic diagram of the experimental design. Lymphoblastoid cells (LCLs) expressing FMR1-217, FXS2 cell line (GM06897, Coriell Institute For Medical Research, Camden, NJ) were incubated with 80 nM of the indicated ASOs (using Lipofectamine™ RNAIMAX®, Thermo Fisher Scientific, Waltham, MA) for 48 hours. Then cells were collected for RNA and protein extraction. FIG. 29B shows FMR1 and FMR1-217 RNA levels as determined by RT-qPCR made relative to GAPDH RNA. Vehicle refers to equivalent volumes of OPTI-MEM® (Thermo Fisher Scientific) medium with Lipofectamine™ RNAIMAX® added without any ASO. *p<0.05).

FIGS. 30A-30B. LCLs (line FXS2) were treated with 2.5 μM of the indicated ASOs (by gymnotic delivery (without lipofectamine)) for 72 hours prior to protein and RNA extraction. FIG. 30A shows FMR1 RNA levels (determined by RT-qPCR and normalized to GAPDH RNA). Control fibroblasts refer to normal (i.e., not Fragile X) fibroblasts. FIG. 30B shows FMRP protein levels (normalized to GAPDH) as determined by western blot analysis. *p<0.05; **p<0.01; ns, not significant.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing some embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Exemplification. When “about” precedes a range, as in “about 24-96 hours,” the term “about” should be read as applying to both of the given values of the range, such that “about 24-96 hours” means about 24 hours to about 96 hours.

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

When introducing elements disclosed herein, the articles “a,” “an,” “the,” and “said” are intended to mean that there are one or more of the elements. Further, the one or more elements may be the same or different. Thus, for example, unless the context clearly indicates otherwise, “an agent” includes a single agent, and two or more agents. Further the two or more agents can be the same or different as, for example, in embodiments wherein a first agent comprises a polynucleotide (e.g., ASO) of a first sequence and a second agent comprises a polynucleotide (e.g., ASO) of a second sequence.

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

In one aspect, the present disclosure provides a method of treating a fragile X-associated disorder, comprising administering to a subject in need thereof, a therapeutically effective amount of an agent that decreases expression of an aberrant fragile X messenger ribonucleoprotein 1 (FMR1) gene product, thereby treating the fragile X-associated disorder in the subject. An agent that decreases expression of an aberrant FMR1 gene product in a method disclosed herein can be any one or more of the agents disclosed herein.

In another aspect, the present disclosure provides a method of treating a fragile X-associated disorder, comprising administering to a subject in need thereof, a therapeutically effective amount of an agent that modulates splicing of an FMR1 gene (e.g., decreasing splicing between Exons 1 and 2 of FMR1-217), thereby treating the fragile X-associated disorder in the subject. An agent that modulates splicing of an FMR1 gene in a method disclosed herein can be any one or more of the agents disclosed herein.

In another aspect, the present disclosure provides a method of decreasing expression of an aberrant FMR1 gene product in a cell, comprising contacting the cell with an agent under conditions whereby the agent is introduced into the cell, thereby decreasing expression of the aberrant FMR1 gene product in the cell.

In another aspect, the present disclosure provides a method of modulating FMR1 splicing and/or expression in a cell, comprising contacting the cell with an agent (e.g., a polynucleotide) under conditions whereby the agent is introduced into the cell, thereby modulating FMR1 splicing and/or expression in the cell. An agent that modulates FMR1 splicing and/or expression in a method disclosed herein can be any one or more of the agents disclosed herein.

In another aspect, the present disclosure provides a method of increasing the level of fragile X messenger ribonucleoprotein (FMRP) in a cell, comprising contacting the cell with an agent (e.g., a polynucleotide) under conditions whereby the agent is introduced into the cell, such that the level of FMRP in the cell is enhanced.

In another aspect, the present disclosure provides a method of reducing CGG triplet repeat expansion in FMR1 5′ UTR in a cell, comprising contacting the cell with an agent that reduces expression of an aberrant FMR1 gene product under conditions whereby the agent is introduced into the cell, thereby reducing CGG triplet repeat expansion in the cell.

### Fragile X-Associated Disorders

Fragile X-associated disorders are caused by mutation of the fragile X messenger ribonucleoprotein 1 (FMR1, previously known as fragile X mental retardation 1) gene, located in the q27.3 locus of the X chromosome. The expansion of the trinucleotide CGG above the normal range (greater than 54 repeats) in the non-coding region of the FMR1 gene has been associated with the development of fragile X-associated disorders. For example, in those carrying the premutation, the trinucleotide CGG can range from 55-200 CGG repeats. In some embodiments, a fragile X-associated disorder described herein is linked to greater than 77 CGG repeats in FMR1, e.g., greater than 98 CGG repeats in FMR1. In some embodiments, the fragile X-associated disorder is linked to at least 140 CGG repeats in FMR1. In some embodiments, the fragile X-associated disorder is linked to at least 201 CGG repeats in FMR1.

Non-limiting examples of fragile X-associated disorders include fragile-X associated tremor/ataxia syndrome (FXTAS), fragile X-associated primary ovarian insufficiency (FXPOI), fragile X-associated neuropsychiatric disorders (FXAND), and fragile X syndrome (FXS). In some embodiments, a fragile X-associated disorder described herein is fragile X syndrome (FXS), fragile X-associated primary ovarian insufficiency (FXPOI), or fragile X-associated tremor/ataxia syndrome (FXTAS), or a combination thereof. In some embodiments, the fragile X-associated disorder is FXS.

### FMR1 Gene Products

A FMR1 gene encodes a fragile X messenger ribonucleoprotein (FMRP, previously known as fragile X mental retardation protein).

In some embodiments, an FMR1 gene described herein is a human FMR1 gene (e.g., corresponding to GenBank reference number NC_000023.11), a mouse FMR1 gene (e.g., NC_000086.8), a rat FMR1 gene (e.g., NC_051356.1), a golden hamster FMR1 gene (e.g., NW_024429188.1), a Chinese hamster FMR1 gene (e.g., NW_003614110.1), a dog FMR1 gene (e.g., NC_051843.1), a pig FMR1 gene (e.g., NC_046383.1), or a monkey FMR1 gene (e.g., NC_041774.1). In some embodiments, the FMR1 gene is a human FMR1 gene. The human FMR1 gene (Ensembl: ENSG00000102081.16) is located within chromosome band Xq27.3 between base pairs 147,911,919 and 147,951,125 (the numberings referring to Genome Reference Consortium Human Build 38 (GRCh38)).

As used herein, “an aberrant FMR1 gene product” refers to an FMR1 gene product elevated in a subject who has, or is predisposed to have a fragile X-associated disorder. In some embodiments, an aberrant FMR1 gene product described herein is elevated in a subject who is being treated, or has been treated, for a fragile X-associated disorder. In some embodiments, the aberrant FMR1 gene product is elevated in a subject having at least 55 CGG repeats in the 5′ untranslated region of an FMR1 gene, for example, having at least 77, at least 78, at least 98, at least 99, at least 140, or at least 201 CGG repeats in the 5′ untranslated region of the FMR1 gene. In some embodiments, the aberrant FMR1 gene product is elevated in a subject having at least 201 CGG repeats in the 5′ untranslated region of an FMR1 gene. In some embodiments, an aberrant FMR1 gene product described herein is not expressed in typically developing subjects (e.g., typically developing humans). In some embodiments, the aberrant FMR1 gene product is elevated in a subject who is a premutation carrier for FXS. In some embodiments, the aberrant FMR1 gene product is elevated in a subject who has FXS.

In some embodiments, an aberrant FMR1 gene product described herein is produced from a CGG expansion-dependent mis-splicing of a FMR1 gene.

In some embodiments, an aberrant FMR1 gene product described herein contributes to pathology of a fragile X-associated disorder described herein. In some embodiments, an aberrant FMR1 transcript, its protein product, or both contribute to pathology of the fragile X-associated disorder. In some embodiments, an aberrant FMR1 transcript described herein contributes to pathology of the fragile X-associated disorder. In some embodiments, a protein encoded by an aberrant FMR1 transcript described herein contributes to pathology of the fragile X-associated disorder. In some embodiments, an aberrant FMR1 transcript and its protein product contribute to pathology of the fragile X-associated disorder.

In some embodiments, an aberrant FMR1 gene product described herein comprises FMR1-217, its protein product, or both. In some embodiments, the aberrant FMR1 gene product comprises FMR1-217. In some embodiments, the aberrant FMR1 gene product comprises the protein product of FMR1-217. In some embodiments, the aberrant FMR1 gene product comprises FMR1-217 and its protein product.

In humans, FMR1-217, also referred to as “isoform 12” or “iso12,” is a transcript corresponding to A0A087X1M7 (ENST00000621447.1, 1,832 nucleotides). FMR1-217 has 2 exons, and the splicing between Exon 1 of FMR1-217 (between base pairs 147,912,123 and 147,912,230, SEQ ID NO:23) and Exon 2 of FMR1-217 (between base pairs 147,912,728 and 147,914,451, SEQ ID NO:21) is considered aberrant FMR1 RNA splicing. FMR1-217 is detected in a subpopulation of subjects with fragile X-associated disorder, including a subpopulation of FXS patients, and a subpopulation of premutation carriers for FXS.

(SEQ ID NO: 23)
CGCCCGCAGCCCACCTCTCGGGGGCGGGCTCCCGGCGCTAGCAGGGCTGA
AGAGAAGATGGAGGAGCTGGTGGTGGAAGTGCGGGGCTCCAATGGCGCTT
TCTACAAG.
(SEQ ID NO: 21)
CATTGGGACTTCGGAGAGCTCCACTGTTCTGGGCGAGGGCTGTGAAGAAA
GAGTAGTAAGAAGCGGTAGTCGGCACCAAATCACAATGGCAACTGATTTT
TAGTGGCTTCTCTTTGTGGATTTCGGAGGAGATTTTAGATCCAAAAGTTT
CAGGAAGACCCTAACATGGCCCAGCAGTGCATTGAAGAAGTTGATCATCG
TGAATATTCGCGTCCCCCTTTTTGTTAAACGGGGTAAATTCAGGAATGCA
CATGCTTCAGCGTCTAAAACCATTAGCAGCGCTGCTACTTAAAAATTGTG
TGTGTGTGTTTAAGTTTCCAAAGACCTAAATATATGCCATGAAACTTCAG
GTAATTAACTGAGAGTATATTATTACTAGGGCATTTTTTTTTTAACTGAG
CGAAAATATTTTTGTGCCCCTAAGAACTTGACCACATTTCCTTTGAATTT
GTGGTGTTGCAGTGGACTGAATTGTTGAGGCTTTATATAGGCATTCATGG
GTTTACTGTGCTTTTTAAAGTTACACCATTGCAGATCAACTAACACCTTT
CAGTTTTAAAAGGAAGATTTACAAATTTGATGTAGCAGTAGTGCGTTTGT
TGGTATGTAGGTGCTGTATAAATTCATCTATAAATTCTCATTTCCTTTTG
AATGTCTATAACCTCTTTCAATAATATCCCACCTTACTACAGTATTTTGG
CAATAGAAGGTGCGTGTGGAAGGAAGGCTGGAAAATAGCTATTAGCAGTG
TCCAACACAATTCTTAAATGTATTGTAGAATGGCTTGAATGTTTCAGACA
GGACACGTTTGGCTATAGGAAAATAAACAATTGACTTTATTCTGTGTTTA
CCAATTTTATGAAGACATTTGGAGATCAGTATATTTCATAAATGAGTAAA
GTATGTAAACTGTTCCATACTTTGAGCACAAAGATAAAGCCTTTTGCTGT
AAAAGGAGGCAAAAGGTAACCCCGCGTTTATGTTCTTAACAGTCTCATGA
ATATGAAATTGTTTCAGTTGACTCTGCAGTCAAAATTTTAATTTCATTGA
TTTTATTGATCCATAATTTCTTCTGGTGAGTTTGCGTAGAATCGTTCACG
GTCCTAGATTAGTGGTTTTGGTCACTAGATTTCTGGCACTAATAACTATA
ATACATATACATATATATGTGTGAGTAACGGCTAATGGTTAGGCAAGATT
TTGATTGACCTGTGATATAAACTTAGATTGGATGCCACTAAAGTTTGCTT
ATCACAGAGGGCAAGTAGCACATTATGGCCTTGAAGTACTTATTGTTCTC
TTCCAGCAACTTATGATTTGCTCCAGTGATTTTGCTTGCACACTGACTGG
AATATAAGAAATGCCTTCTATTTTTGCTATTAATTCCCTCCTTTTTTGTT
TTGTTTTGTAACGAAGTTGTTTAACTTGAAGGTGAATGAAGAATAGGTTG
GTTGCCCCTTAGTTCCCTGAGGAGAAATGTTAATACTTGAACAAGTGTGT
GTCAGACAAATTGCTGTTATGTTTATTTAATTAAGTTTGATTTCTAAGAA
AATCTCAAATGGTCTGCACTGATGGAAGAACAGTTTCTGTAACAAAAAAG
CTTGAAATTTTTATATGACTTATAATACTGCTGTGAGTTTTAAAAGTAAA
GCAAAAGTAAACTGAGTTGCTTGTCCAGTGGGATGGACAGGAAAGATGTG
AAATAAAAACCAATGAAAAATGAA.

FMR1-217 encodes a 31-amino acid protein (SEQ ID NO:22)).

(SEQ ID NO: 22)
MEELVVEVRGSNGAFYKHWDFGELHCSGRGL.

Additional information on FMR1-217 and its protein product, can be found at the web address below, the contents of which are incorporated herein by reference in their entirety: useast.ensembl.org/Homo_sapiens/Transcript/Summary?db=core;g=ENSG00000102081; r=X:147911951-147951125;t=ENST00000621447.

In some embodiments, a method disclosed herein increases the level of expression of FMRP in a subject described herein. In some embodiments, a method disclosed herein increases the level of expression of FMRP in a cell described herein.

In some embodiments, a method disclosed herein increases a normal FMR1 gene product (e.g., a normal FMR1 transcript, its protein product, or both) in a subject and/or cell described herein.

Several normal FMR1 gene products are expressed in typically developing subjects (e.g., humans who do not have FXS). Non-limiting examples of “normal” human FMR1 gene products include:
a transcript corresponding to Q06787 (FMR1-205, ENST00000370475.9, 4,441 nucleotides), and its protein product (a 632-amino acid protein (NP_002015.1)),
a transcript corresponding to NM_001185075.2 (4,170 nucleotides), and its protein product (a 537-amino acid protein (NP_001172004.1)),
a transcript corresponding to NM_001185076.2 (4,378 nucleotides), and its protein product (a 611-amino acid protein (NP_001172005.1)),
a transcript corresponding to NM_001185082.2 (4,303 nucleotides), and its protein product (a 586-amino acid protein (NP_001172011.1)),
a transcript corresponding to NM_001185081.2 (4,107 nucleotides), and its protein product (a 516-amino acid protein (NP_001172010.1)),
a transcript corresponding to Q06787-9 (FMR1-201, ENST00000218200.12, 4,333 nucleotides), and its protein product (a 611-amino acid protein),
a transcript corresponding to Q06787-8 (FMR1-208, ENST00000440235.6, 4,271 nucleotides), and its protein product (a 586-amino acid protein),
a transcript corresponding to X5D907 (FMR1-223, ENST00000687593.1, 4,159 nucleotides), and its protein product (a 594-amino acid protein),
a transcript corresponding to Q06787-10 (FMR1-204, ENST00000370471.7, 4,125 nucleotides), and its protein product (a 537-amino acid protein),
a transcript corresponding to G3V0J0 (FMR1-207, ENST00000439526.6, 3,699 nucleotides), and its protein product (a 592-amino acid protein),
a transcript corresponding to A8MQB8 (FMR1-206, ENST00000370477.5, 3,437 nucleotides), and its protein product (a 582-amino acid protein),
a transcript corresponding to A0A087WY29 (FMR1-212, ENST00000495717.6, 2,874 nucleotides), and its protein product (a 561-amino acid protein),
a transcript corresponding to A0A087WXI3 (FMR1-214, ENST00000616382.5, 2,799 nucleotides), and its protein product (a 536-amino acid protein), and
a transcript corresponding to R9WNI0 (“FMR1-218”, ENST00000621453.5, 1,827 nucleotides), and its protein product (a 548-amino acid protein).

In some embodiments, a normal FMR1 gene product described herein comprises a transcript corresponding to Q06787 (FMR1-205, ENST00000370475.9, 4,441 nucleotides), and its protein product (a 632-amino acid protein (NP_002015.1)). FMR1-205, also referred to as “isoform 1” or “iso1”, is produced in typical developing individuals and a subpopulation of FXS subjects. FMR1-205 has 17 exons, and the splicing between Exon 1 of FMR1-205 (between base pairs 147,911,919 and 147,912,230, SEQ ID NO:19) and Exon 2 of FMR1-205 (between base pairs 147,921,933 and 147,921,985, SEQ ID NO:20) is considered normal FMR1 RNA splicing. Additional information on FMR1-205 and its protein product, can be found at the web address below, the contents of which are incorporated herein by reference in their entirety: useast.ensembl.org/Homo_sapiens/Transcript/Summary?db=core;g=ENSG00000102081;r=X:14 7911951-147951125;t=ENST00000370475.

(SEQ ID NO: 19)
CTCAGTCAGGCGCTCAGCTCCGTTTCGGTTTCACTTCCGGTGGAGGGCCG
CCTCTGAGCGGGCGGCGGGCCGACGGCGAGCGCGGGCGGCGGCGGTGACG
GAGGCGCCGCTGCCAGGGGGCGTGCGGCAGCGCGGCGGCGGCGGCGGCGG
CGGCGGCGGCGGAGGCGGCGGCGGCGGCGGCGGCGGCGGCGGCTGGGCCT
CGAGCGCCCGCAGCCCACCTCTCGGGGGCGGGCTCCCGGCGCTAGCAGGG
CTGAAGAGAAGATGGAGGAGCTGGTGGTGGAAGTGCGGGGCTCCAATGGC
GCTTTCTACAAG.
(SEQ ID NO: 20)
GCATTTGTAAAGGATGTTCATGAAGATTCAATAACAGTTGCATTTGAAAA
CAA.

### Agents

In another aspect, the present disclosure provides an agent that modulates splicing and/or expression of FMR1 gene (e.g., decreasing splicing between Exons 1 and 2 of FMR1-217 or decreasing a protein encoded by FMR1-217).

In another aspect, the present disclosure provides an agent that modulates splicing and/or expression of FMR1 gene (e.g., decreasing splicing between Exons 1 and 2 of FMR1-217 or decreasing a protein encoded by FMR1-217).

In another aspect, the present disclosure provides an agent that decreases expression of an aberrant FMR1 gene product.

As used herein, the term “decreasing,” “decrease,” “reducing” or “reduce” refers to modulation that results in a lower level of the aberrant FMR1 gene product (e.g., FMR1-217 and/or its protein product), relative to a reference (e.g., the level prior to or in an absence of modulation by an agent disclosed herein).

In some embodiments, an agent disclosed herein (e.g., an anti-sense RNA polynucleotide) decreases expression of an aberrant FMR1 gene product (e.g., FMR1-217 and/or its protein product), relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95% or 98%.

In some embodiments, an agent disclosed herein (e.g., an anti-sense RNA polynucleotide) decreases expression of an aberrant FMR1 transcript, decreases expression of an aberrant FMR1-encoded protein, or both.

In some embodiments, an agent disclosed herein (e.g., an anti-sense RNA polynucleotide) decreases expression of an aberrant FMR1 transcript (e.g., FMR1-217). In some embodiments, the agent decreases expression of the aberrant FMR1 transcript, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95% or 98%.

In some embodiments, an agent disclosed herein (e.g., an anti-sense RNA polynucleotide) decreases expression of an aberrant FMR1-encoded protein (e.g., the protein product of FMR1-217). In some embodiments, the agent decreases expression of the aberrant FMR1-encoded protein, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95% or 98%.

In some embodiments, an agent disclosed herein (e.g., an anti-sense RNA polynucleotide) decreases expression of an aberrant FMR1 transcript and an aberrant FMR1-encoded protein (e.g., FMR1-217 and its protein product). In some embodiments, the agent decreases expression of the aberrant FMR1 transcript and the aberrant FMR1-encoded protein, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95% or 98%.

An agent disclosed herein may decrease expression of an aberrant FMR1 gene product directly or indirectly, for example, by altering transcription initiation, transcription elongation, transcription termination, RNA splicing, RNA processing, RNA stability, translation initiation, post-translational modification, protein stability, protein degradation, or a combination of the foregoing.

In some embodiments, an agent disclosed herein (e.g., an anti-sense RNA polynucleotide) decreases splicing of an aberrant FMR1 transcript (e.g., between Exons 1 and 2 of FMR1-217). In some embodiments, the agent decreases splicing of the aberrant FMR1 transcript, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95% or 98%.

In some embodiments, an agent disclosed herein (e.g., an anti-sense RNA polynucleotide) increases the level of expression of FMRP. As used herein, the term “increasing” or “increase” refers to modulation that results in a higher level of FMRP, relative to a reference (e.g., the level prior to or in an absence of modulation by an agent disclosed herein).

In some embodiments, an agent disclosed herein (e.g., an anti-sense RNA polynucleotide) increases FMRP expression, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%.

In some embodiments, an agent disclosed herein (e.g., an anti-sense RNA polynucleotide) increases expression of a normal FMR1 gene product, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%. In some embodiments, the agent increases expression of a normal FMR1 gene product to at least 5% of the level observed in in typically developing subjects (e.g., human), e.g., at least: 10%, 15%, 20%, 25%, 30%, 35%, 40%, 45%, 50%, 55%, 60%, 65%, 70%, 75%, 80%, 85%, 90%, 95%, 98%, 99%, or 100%, of the level observed in the typically developing subject. In some embodiments, the agent increases expression of a normal FMR1 gene product to at least 30% of the level observed in in typically developing subjects (e.g., human).

In some embodiments, an agent disclosed herein (e.g., an anti-sense RNA polynucleotide) increases expression of a normal FMR1 transcript, a normal FMR1-encoded protein, or both.

In some embodiments, an agent disclosed herein (e.g., an anti-sense RNA polynucleotide) increases expression of a normal FMR1 transcript (e.g., FMR1-205). In some embodiments, the agent increases expression of the normal FMR1 transcript, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%.

In some embodiments, an agent disclosed herein (e.g., an anti-sense RNA polynucleotide) increases expression of a normal FMR1-encoded protein (e.g., a protein encoded by FMR1-205). In some embodiments, the agent increases expression of the normal FMR1-encoded protein, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%.

In some embodiments, an agent disclosed herein (e.g., an anti-sense RNA polynucleotide) increases expression of a normal FMR1 transcript and a normal FMR1-encoded protein (e.g., FMR1-205 and its protein product). In some embodiments, the agent increases expression of the normal FMR1 transcript and the normal FMR1-encoded protein, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%.

In some embodiments, an agent disclosed herein (e.g., an anti-sense RNA polynucleotide) increases splicing of a normal FMR1 transcript (e.g., between Exons 1 and 2 of FMR1-205). In some embodiments, the agent increases splicing of the normal FMR1 transcript, relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%.

In some embodiments, an agent disclosed herein (e.g., an anti-sense RNA polynucleotide) decreases expression of an aberrant FMR1 gene product (e.g., FMR1-217 and/or its protein product) and increases expression of FMRP.

In some embodiments, an agent disclosed herein (e.g., an anti-sense RNA polynucleotide) decreases expression of an aberrant FMR1 gene product (e.g., FMR1-217 and/or its protein product) and increases expression of a normal FMR1 gene product (e.g., FMR1-205 and/or its protein product). In some embodiments, an agent disclosed herein (e.g., an anti-sense RNA polynucleotide) decreases expression of an aberrant FMR1 transcript, decreases expression of an aberrant FMR1-encoded protein, increases expression of a normal FMR1 transcript, increases expression of a normal FMR1-encoded protein, or a combination thereof.

In some embodiments, an agent disclosed herein (e.g., an anti-sense RNA polynucleotide):
decreases expression of an aberrant FMR1 gene product (e.g., FMR1-217 and/or its protein product), relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95% or 98%; and
increases expression of a normal FMR1 gene product (e.g., FMR1-205 and/or its protein product), relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%.

In some embodiments, an agent disclosed herein (e.g., an anti-sense RNA polynucleotide):
decreases splicing of an aberrant FMR1 transcript (e.g., between Exons 1 and 2 of FMR1-217), relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95% or 98%; and
increases splicing of a normal FMR1 transcript (e.g., between Exons 1 and 2 of FMR1-205), relative to a reference, by at least 5%, e.g., by at least: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125%.

In some embodiments, a level of an FMR1 gene product (e.g., an aberrant FMR1 transcript, an aberrant FMR1-encoded protein, a normal FMR1 transcript, a normal FMR1-encoded protein, or a combination thereof), is measured at least 1 day after an agent disclosed herein is administered to a subject, e.g., for at least: 2 days, 3 days, 4 days, 5 days, 6 days, 8 days, 9 days, 10 days, 1 week, 2 weeks, 3 weeks, 1 month, 2 months, 3 months, 4 months, 5 months or 6 months, after a treatment with an agent disclosed herein has begun.

In some embodiments, a level an FMR1 gene product is measured in a tissue or a cell. In some embodiments, a level an FMR1 gene product is measured in a white blood cell. In some embodiments, a level an FMR1 gene product is measured in a leukocyte. In some embodiments, a level an FMR1 gene product is measured in a fibroblast cell (e.g., a dermal derived fibroblast cell or a lung-derived fibroblast cell). In some embodiments, a level an FMR1 gene product is measured in a cortex tissue (e.g., a brain biopsy of superficial cortex).

### Target Sequences

In some embodiments, an agent disclosed herein (e.g., an antisense oligonucleotide (ASO)) promotes exclusion of an aberrant FMR1 exon. In some embodiments, the agent promotes exclusion of Exon 2 of FMR1-217.

In some embodiments, an agent disclosed herein (e.g., an ASO) targets (indirectly, or directly, e.g., binds) a primary aberrant transcript (pre-mRNA) of an FMR1 gene. As used herein, the term “target” refers to a preliminary mRNA region, and specifically, to a region identified by Exon 2, and the adjacent intron 1-2 regions of FMR1-217, which is responsible for the splicing associated with FMR1-217. In some embodiments, a target sequence refers to a portion of the target RNA against which a polynucleotide (e.g., an ASO) is directed, that is, the sequence to which the polynucleotide will hybridize by Watson-Crick base pairing of a complementary sequence.

In some embodiments, the agent targets a contiguous nucleotide sequence within pre-mRNA of FMR1-217, wherein the contiguous nucleotide sequence is at least 8 nucleotides in length. In some embodiments, the contiguous nucleotide sequence is at least 9 nucleotides in length, for example, at least: 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 or 80 nucleotides in length. In some embodiments, the contiguous nucleotide sequence is at least 12 nucleotides in length. In some embodiments, the contiguous nucleotide sequence is about 8-80 nucleotides in length, for example, about: 10-60, 10-40, 10-30, 12-80, 12-60, 12-40, 12-38, 12-30, 13-38, 13-36, 14-36, 14-34, 15-80, 15-60, 15-40, 15-34, 15-32, 16-32, 16-30, 17-30, 17-28, 18-28, 18-26, 19-26, 19-24, 20-80, 20-60, 20-40, 20-30, 20-24 or 20-22 nucleotides in length. In some embodiments, the contiguous nucleotide sequence is about 10-30 nucleotides in length.

In some embodiments, the agent (e.g., an ASO) targets a contiguous nucleotide sequence within SEQ ID NO:24 (e.g., within any one or more of SEQ ID NOs:25-42), wherein the contiguous nucleotide sequence is at least 8 nucleotides in length. In some embodiments, the agent (e.g., an ASO) targets a contiguous nucleotide sequence within SEQ ID NO:27, wherein the contiguous nucleotide sequence is at least 8 nucleotides in length. In some embodiments, the contiguous nucleotide sequence is at least 9 nucleotides in length, for example, at least: 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 or 80 nucleotides in length.

(SEQ ID NO: 24)
UCAGGUCUCCUUUGGCUUCUCUUUUCCGGUCUAGCAUUGGGACUUCGGAG
AGCUCCACUGUUCUGGGCGAGGGCUGUGAAGAAAGA.
(SEQ ID NO: 25)
UCAGGUCUCCUUUGGCUUCUCUUUUCCGGUCUAGCAUUGGGACUUCGG
AGA
(SEQ ID NO: 26)
CAUUGGGACUUCGGAGAGCUCCACUGUUCUGGGCGAGGGCUGUGAAGA
AAGA
(SEQ ID NO: 27)
UGGGACUUCGGAGAGCUCCACUGUUCUGGGCGAGGGCUGUGAAGAA

In some embodiments, the agent (e.g., an ASO) targets a contiguous nucleotide sequence within FMR1-217 Exon 2, FMR1-217 Intron 1-2, the junction between Exon 2 and Intron 1-2 of FMR1-217, or a combination thereof. In some embodiments, the agent (e.g., an ASO) targets a contiguous nucleotide sequence within any one or more of SEQ ID NOs:28-42, wherein the contiguous nucleotide sequence is at least 8 nucleotides in length. In some embodiments, the agent (e.g., an ASO) targets a contiguous nucleotide sequence within any one or more of SEQ ID NOs:37-42, wherein the contiguous nucleotide sequence is at least 8 nucleotides in length. In some embodiments, the contiguous nucleotide sequence is selected from a polynucleotide sequence set forth in any one of SEQ ID NOs:28-42. In some embodiments, the contiguous nucleotide sequence is selected from a polynucleotide sequence set forth in any one of SEQ ID NOs:37-42.

(SEQ ID NO: 28)
UCAGGUCUCCUUUGGCUUCU
(SEQ ID NO: 29)
GUCUCCUUUGGCUUCUCUUU
(SEQ ID NO: 30)
UGGCUUCUCUUUUCCGGUCUAG
(SEQ ID NO: 31)
UUCUCUUUUCCGGUCUAGCAU
(SEQ ID NO: 32)
UCUUUUCCGGUCUAGCAUUG
(SEQ ID NO: 33)
UCCGGUCUAGCAUUGGGACUU
(SEQ ID NO: 34)
UAGCAUUGGGACUUCGGAGA
(SEQ ID NO: 35)
UGGGACUUCGGAGAGCUC
(SEQ ID NO: 36)
UCGGAGAGCUCCACUGUUCU
(SEQ ID NO: 37)
GAGCUCCACUGUUCUGGGCG
(SEQ ID NO: 38)
CUCCACUGUUCUGGGCGAGG
(SEQ ID NO: 39)
GGACUUCGGAGAGCUCCACUG
(SEQ ID NO: 40)
GGAGAGCUCCACUGUUCUGGG
(SEQ ID NO: 41)
UGUUCUGGGCGAGGGCUGUG
(SEQ ID NO: 42)
UGGGCGAGGGCUGUGAAGAA

### Polynucleotides (Polynucleotide Agents)

In some embodiments, an agent disclosed herein comprises at least one polynucleotide disclosed herein. In some embodiments, the agent comprises at least two polynucleotides disclosed herein.

In another aspect, the present disclosure provides a polynucleotide capable of decreasing expression of an aberrant FMR1 gene product.

In another aspect, the present disclosure provides a polynucleotide capable of decreasing splicing of FMR1-217.

In another aspect, the present disclosure provides a method of enhancing the level of FMRP in a cell, comprising contacting the cell with an oligonucleotide which is complementary to at least 8 contiguous nucleotides of a sequence set forth in SEQ ID NOs:24-42, such that the level of FMRP in the cell is enhanced.

As used herein, a “polynucleotide” is defined as a plurality of nucleotides and/or nucleotide analogs linked together in a single molecule. In some embodiments, a polynucleotide disclosed herein comprises deoxyribonucleotides. In some embodiments, the polynucleotide comprises ribonucleotides. Non-limiting examples of polynucleotides include single-, double- or multi-stranded DNA or RNA, DNA-RNA hybrids (e.g., each “T” position may be independently substituted by a “U” or vice versa), or a polymer comprising purine and pyrimidine bases, or other natural, chemically or biochemically modified, non-natural, or derivatized nucleotide bases. The backbone of the polynucleotide can comprise sugars and phosphate groups, modified or substituted sugar or phosphate groups, a polymer of synthetic subunits such as phosphoramidates, or a combination thereof.

As used herein, the term “nucleotide analog” or “altered nucleotide” or “modified nucleotide” refers to a non-standard nucleotide, including non-naturally occurring ribonucleotides or deoxyribonucleotides. A nucleotide analog may be modified at any position so as to alter certain chemical properties of the nucleotide yet retain the ability to perform its intended function. Non-limiting examples of positions of the nucleotide which may be derivatized include the 5 position, e.g., 5-(2-amino)propyl uridine, 5-bromo uridine, 5-propyne uridine, and 5-propenyl uridine; the 6 position, e.g., 6-(2-amino)propyl uridine; the 8-position for adenosine and/or guanosines, e.g., 8-bromo guanosine, 8-chloro guanosine, and 8-fluoroguanosine. Nucleotide analogs also include deaza nucleotides, e.g., 7-deaza-adenosine; O- and N-modified (e.g., alkylated or N6-methyl adenosine) nucleotides.

In some embodiments, a nucleotide analog comprises a modification to the sugar portion of the nucleotide. For example, the 2′ OH— group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl or aryl.

In some embodiments, a phosphate group of the nucleotide is modified, e.g., by substituting one or more of the oxygens of the phosphate group with sulfur (e.g., phosphorothioates). In some embodiments, the ASO is a phosphorothioate-modified polynucleotide, such as a polynucleotide where each internucleotide linkage is a phosphorothioate, or where at least half of the internucleotide linkages are phosphorothioate.

In some embodiments, a polynucleotide disclosed herein (e.g., ASO) binds a target sequence described herein.

In some embodiments, a targeting polynucleotide disclosed herein (e.g., ASO) has near or substantial complementarity to a target sequence described herein. In some embodiments, the polynucleotide is formed of contiguous complementary sequences (to the target sequence). In some embodiments, the polynucleotide sequence is formed of non-contiguous complementary sequences (to the target sequence), for example, when placed together, constitute sequence that spans the target sequence.

In some embodiments, a polynucleotide disclosed herein (e.g., ASO) comprises a nucleotide sequence that is complementary (e.g., fully complementary or partially complementary) to a target sequence described herein (such that the polynucleotide is capable of hybridizing or annealing to target sequence, e.g., under physiological conditions). As used herein, “complementary” refers to sequence complementarity between two different polynucleotides or between two regions of the same polynucleotide. A first region of a polynucleotide is complementary to a second region of the same or a different polynucleotide if, when the two regions are arranged in an anti-parallel fashion, at least one nucleotide residue of the first region is capable of base pairing (i.e., hydrogen bonding) with a residue of the second region, thus forming a hydrogen-bonded duplex.

In some embodiments, a polynucleotide disclosed herein (e.g., ASO) specifically hybridizes to a target polynucleotide described herein (e.g., contiguous nucleotides of a sequence set forth in SEQ ID NOs:24-42), for example, under physiological conditions, with a Tm of at least 45° C., e.g., at least: 50° C., 55° C., 60° C., 65° C., 70° C., 75° C. or 80° C. The Tm is the temperature at which 50% of a target sequence hybridizes to a complementary polynucleotide at a given ionic strength and pH. In some embodiments, specific hybridization corresponds to stringent hybridization conditions. In some embodiments, specific hybridization occurs with near complementary of the antisense oligomer to the target sequence. In some embodiments, specific hybridization occurs with substantial complementary of the antisense oligomer to the target sequence. In some embodiments, specific hybridization occurs with exact complementary of the antisense oligomer to the target sequence.

In some embodiments, a polynucleotide disclosed herein (e.g., ASO) comprises a nucleotide sequence that is complementary to a contiguous nucleotide sequence (e.g., 10 to 30 nucleotides) of pre-mRNA of an aberrant FMR1 transcript.

In some embodiments, a polynucleotide disclosed herein (e.g., ASO) comprises a nucleotide sequence that is complementary to a contiguous nucleotide sequence (e.g., 10 to 30 nucleotides) of pre-mRNA of FMR1-217. In some embodiments, the polynucleotide comprises a nucleotide sequence that is complementary to a target sequence within any one of SEQ ID NOs:24-42 (e.g., any one of SEQ ID NOs:24-27, any one of SEQ ID NOs:28-42, or a combination thereof).

In some embodiments, a polynucleotide disclosed herein is an antisense oligonucleotide (ASO). In some embodiments, the polynucleotide is a small interfering RNA (siRNA), a short hairpin RNA (shRNA), an antisense DNA, an antisense RNA, a microRNA (miRNA), an antagomir, a guide RNA (gRNA). The polynucleotide may be modified, including with one or more locked nucleic acid (LNA) nucleotides, one or more 2′-modified ribonucleotides, one or more morpholino nucleotides, or a combination thereof.

In some embodiments, a polynucleotide disclosed herein (e.g., ASO) comprises a nucleotide sequence specifically hybridizes to (e.g., having near, substantial, or exact complementarity to) at least a portion of X chromosome between base pairs 147,911,919 and 147,921,985 (e.g., a target sequence within X chromosome between base pairs 147,911,919 and 147,921,985), for example, between 147,911,919 and 147,921,933, between 147,911,919 and 147,912,230, between 147,911,919 and 147,912,123, between 147,911,919 and 147,914,451, between 147,911,919 and 147,912,728, between 147,912,231 and 147,921,932, between 147,912,231 and 147,914,451, between 147,912,231 and 147,912,727, between 147,912,728 and 147,914,451, between 147,912,694 and 147,912,727, between 147,912,710 and 147,912,745, between 147,912,731 and 147,912,766, or between 147,912,694 and 147,912,766. In some embodiments, a polynucleotide disclosed herein (e.g., ASO) has exact complementarity to at least a portion of X chromosome between base pairs 147,911,919 and 147,921,985, for example, between 147,911,919 and 147,921,933, between 147,911,919 and 147,912,230, between 147,911,919 and 147,912,123, between 147,911,919 and 147,914,451, between 147,911,919 and 147,912,728, between 147,912,231 and 147,921,932, between 147,912,231 and 147,914,451, between 147,912,231 and 147,912,727, between 147,912,728 and 147,914,451, between 147,912,694 and 147,912,727, between 147,912,710 and 147,912,745, between 147,912,731 and 147,912,766, or between 147,912,694 and 147,912,766.

In some embodiments, the polynucleotide comprises a nucleotide sequence specifically hybridizes to (e.g., having near, substantial, or exact complementarity to) at least a portion of X chromosome between base pairs 147,912,694 and 147,912,766, for example, having at least about: 70%, 75%, 80%, 85%, 90%, 95%, 96%, 97%, 98%, 99% or 100% sequence identity to the reverse and complementary sequence of the at least a portion of X chromosome between base pairs 147,912,694 and 147,912,766.

As used herein, the term “sequence identity,” refers to the extent to which two nucleotide sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

In some embodiments, the polynucleotide comprises a nucleotide sequence having at least about: 70%, 75%, 80%, 85%, 90%, 95%, 96%, 97%, 98%, 99% or 100% sequence identity to at least a portion of X chromosome between base pairs 147,912,694 and 147,912,766. In some embodiments, the polynucleotide comprises a nucleotide sequence having about: 70%, 75%, 80%, 85%, 90%, 95%, 96%, 97%, 98%, 99% or 100% sequence identity to at least a portion of X chromosome between base pairs 147,912,694 and 147,912,766. In some embodiments, the polynucleotide comprises a nucleotide sequence having about 70-100% sequence identity to at least a portion of X chromosome between base pairs 147,912,694 and 147,912,766, for example, about: 75-100%, 75-99%, 80-100%, 80-98%, 85-100%, 85-97%, 90-100%, 90-96%, 95-100%, 96-100%, 97-100%, 98-100% or 99-100%. In some embodiments, the polynucleotide comprises a nucleotide sequence that is identical to at least a portion of X chromosome between base pairs 147,912,694 and 147,912,766.

In some embodiments, a polynucleotide disclosed herein comprises a nucleotide sequence specifically hybridizes to (e.g., having near, substantial, or exact complementarity to) at least a portion of X chromosome between base pairs 147,912,731 and 147,912,766, for example, having at least about: 70%, 75%, 80%, 85%, 90%, 95%, 96%, 97%, 98%, 99% or 100% sequence identity to the reverse and complementary sequence of the at least a portion of X chromosome between base pairs 147,912,731 and 147,912,766.

In some embodiments, the polynucleotide comprises a nucleotide sequence having at least about: 70%, 75%, 80%, 85%, 90%, 95%, 96%, 97%, 98%, 99% or 100% sequence identity to at least a portion of X chromosome between base pairs 147,912,731 and 147,912,766. In some embodiments, the polynucleotide comprises a nucleotide sequence having about: 70%, 75%, 80%, 85%, 90%, 95%, 96%, 97%, 98%, 99% or 100% sequence identity to at least a portion of X chromosome between base pairs 147,912,731 and 147,912,766. In some embodiments, the polynucleotide comprises a nucleotide sequence having about 70-100% sequence identity to at least a portion of X chromosome between base pairs 147,912,731 and 147,912,766, for example, about: 75-100%, 75-99%, 80-100%, 80-98%, 85-100%, 85-97%, 90-100%, 90-96%, 95-100%, 96-100%, 97-100%, 98-100% or 99-100%. In some embodiments, the polynucleotide comprises a nucleotide sequence that is identical to at least a portion of X chromosome between base pairs 147,912,731 and 147,912,766.

In some embodiments, a polynucleotide disclosed herein (e.g., ASO) comprises a nucleotide sequence having at least 70% sequence identity to, for example, at least: 75%, 80%, 85%, 90%, 95%, 96%, 97%, 98% or 99% sequence identity to a sequence set forth in any one of SEQ ID NOs:1-11 and SEQ ID NOs:43-50. In certain embodiments, the polynucleotide (e.g., ASO) comprises a nucleotide sequence having about: 70%, 75%, 80%, 85%, 90%, 95%, 96%, 97%, 98% or 99% sequence identity to a sequence set forth in any one of SEQ ID NOs:1-11 and SEQ ID NOs:43-50. In some embodiments, the polynucleotide (e.g., ASO) has about: 70%, 75%, 80%, 85%, 90%, 95%, 96%, 97%, 98% or 99% sequence identity to a sequence set forth in any one of SEQ ID NOs:1-11 and SEQ ID NOs:43-50. In some embodiments, the polynucleotide (e.g., ASO) comprises a nucleotide sequence having about 70-100% sequence identity to a sequence set forth in any one of SEQ ID NOs:1-11 and SEQ ID NOs:43-50, for example, about: 75-100%, 75-99%, 80-100%, 80-98%, 85-100%, 85-97%, 90-100%, 90-96%, 95-100%, 96-100%, 97-100%, 98-100% or 99-100%. In some embodiments, the polynucleotide (e.g., ASO) comprises a nucleotide sequence that is identical to a sequence set forth in any one of SEQ ID NOs:1-11 and SEQ ID NOs:43-50. In the sequences, each nucleobase shown as T may independently be T or U. Similarly, each C nucleotide may independently be C or a C analogue such as 5-methyl C, or other substituted C analogue. Other modified nucleobases with equivalent Watson-Crick base pairing properties will be known to one of skill in the art and would also be appropriate for use in the polynucleotides of the instant invention.

(W-704)
(SEQ ID NO: 1)
AGAAGCCAAAGGAGACCTGA.
(W-705)
(SEQ ID NO: 2)
AAAGAGAAGCCAAAGGAGAC.
(W-706)
(SEQ ID NO: 3)
CTAGACCGGAAAAGAGAAGCCA.
(W-707)
(SEQ ID NO: 4)
ATGCTAGACCGGAAAAGAGAA.
(W-708)
(SEQ ID NO: 5)
CAATGCTAGACCGGAAAAGA.
(W-709)
(SEQ ID NO: 6)
AAGTCCCAATGCTAGACCGGA.
(W-710)
(SEQ ID NO: 7)
TCTCCGAAGTCCCAATGCTA.
(W-711)
(SEQ ID NO: 8)
GAGCTCTCCGAAGTCCCA.
(W-712)
(SEQ ID NO: 9)
AGAACAGTGGAGCTCTCCGA.
(W-713)
(SEQ ID NO: 10)
CGCCCAGAACAGTGGAGCTC.
(W-714)
(SEQ ID NO: 11)
CCTCGCCCAGAACAGTGGAG.
(2831)
(SEQ ID NO: 43)
CAGTGGAGCTCTCCGAAGTCC.
(2832)
(SEQ ID NO: 44)
CCCAGAACAGTGGAGCTCTCC.
(2833)
(SEQ ID NO: 45)
CACAGCCCTCGCCCAGAACA.
(2834)
(SEQ ID NO: 46)
TTCTTCACAGCCCTCGCCCA.
(SEQ ID NO: 47)
TCTTTCTTCACAGCCCTCGCCCAGAACAGTGGAGCTCTCCGAAGTCCCAA
TGCTAGACCGGAAAAGAGAAGCCAAAGGAGACCTGA.
(SEQ ID NO: 48)
TCTCCGAAGTCCCAATGCTAGACCGGAAAAGAGAAGCCAAAGGAGACCTG
A.
(SEQ ID NO: 49)
TCTTTCTTCACAGCCCTCGCCCAGAACAGTGGAGCTCTCCGAAGTCCCAA
TG.
(SEQ ID NO: 50)
TTCTTCACAGCCCTCGCCCAGAACAGTGGAGCTCTCCGAAGTCCCA.

In some embodiments, a polynucleotide disclosed herein (e.g., ASO) comprises a nucleotide sequence having at least 70% sequence identity to, for example, at least: 75%, 80%, 85%, 90%, 95%, 96%, 97%, 98% or 99% sequence identity to a sequence set forth in any one of SEQ ID NOs:10-11 and SEQ ID NOs:43-46. In certain embodiments, the polynucleotide (e.g., ASO) comprises a nucleotide sequence having about: 70%, 75%, 80%, 85%, 90%, 95%, 96%, 97%, 98% or 99% sequence identity to a sequence set forth in any one of SEQ ID NOs:1-11 and SEQ ID NOs:43-50. In some embodiments, the polynucleotide (e.g., ASO) has about: 70%, 75%, 80%, 85%, 90%, 95%, 96%, 97%, 98% or 99% sequence identity to a sequence set forth in any one of SEQ ID NOs:10-11 and SEQ ID NOs:43-46. In some embodiments, the polynucleotide (e.g., ASO) comprises a nucleotide sequence having about 70-100% sequence identity to a sequence set forth in any one of SEQ ID NOs:10-11 and SEQ ID NOs:43-46, for example, about: 75-100%, 75-99%, 80-100%, 80-98%, 85-100%, 85-97%, 90-100%, 90-96%, 95-100%, 96-100%, 97-100%, 98-100% or 99-100%. In some embodiments, the polynucleotide (e.g., ASO) comprises a nucleotide sequence that is identical to a sequence set forth in any one of SEQ ID NOs:10-11 and SEQ ID NOs:43-46.

In some embodiments, an agent disclosed herein comprises a first polynucleotide (e.g., ASO) comprising a nucleotide sequence having at least 70% sequence identity, for example, at least: 75%, 80%, 85%, 90%, 95%, 96%, 97%, 98% or 99% sequence identity, to SEQ ID NO:10, and a second polynucleotide (e.g., ASO) comprising a nucleotide sequence having at least 70% sequence identity, for example, at least: 75%, 80%, 85%, 90%, 95%, 96%, 97%, 98% or 99% sequence identity. to SEQ ID NO:11. In some embodiments, the first polynucleotide (e.g., ASO) comprises a nucleotide sequence having about: 70%, 75%, 80%, 85%, 90%, 95%, 96%, 97%, 98% or 99% sequence identity to SEQ ID NO:10, and the second polynucleotide (e.g., ASO) comprises a nucleotide sequence having about: 70%, 75%, 80%, 85%, 90%, 95%, 96%, 97%, 98% or 99% sequence identity to SEQ ID NO:11. In some embodiments, the first polynucleotide (e.g., ASO) comprises a nucleotide sequence having about 70-100% sequence identity to SEQ ID NO:10, for example, about: 75-100%, 75-99%, 80-100%, 80-98%, 85-100%, 85-97%, 90-100%, 90-96%, 95-100%, 96-100%, 97-100%, 98-100% or 99-100%, sequence identity to SEQ ID NO:10; and the second polynucleotide (e.g., ASO) comprises a nucleotide sequence having about 70-100% sequence identity to SEQ ID NO:11, for example, about: 75-100%, 75-99%, 80-100%, 80-98%, 85-100%, 85-97%, 90-100%, 90-96%, 95-100%, 96-100%, 97-100%, 98-100% or 99-100% sequence identity to SEQ ID NO:11. In some embodiments, the first polynucleotide (e.g., ASO) comprises a nucleotide sequence that is identical to SEQ ID NO:10, and the second polynucleotide comprises a nucleotide sequence that is identical to SEQ ID NO:11.

In some embodiments, a polynucleotide disclosed herein (e.g., ASO) comprises a nucleotide sequence having at least 70% sequence identity to, for example, at least: 75%, 80%, 85%, 90%, 95%, 96%, 97%, 98% or 99% sequence identity to a sequence set forth in any one of SEQ ID NOs:51-69. In certain embodiments, the polynucleotide (e.g., ASO) comprises a nucleotide sequence having about: 70%, 75%, 80%, 85%, 90%, 95%, 96%, 97%, 98% or 99% sequence identity to a sequence set forth in any one of SEQ ID NOs:51-69. In some embodiments, the polynucleotide (e.g., ASO) has about: 70%, 75%, 80%, 85%, 90%, 95%, 96%, 97%, 98% or 99% sequence identity to a sequence set forth in any one of SEQ ID NOs:51-69. In some embodiments, the polynucleotide (e.g., ASO) comprises a nucleotide sequence having about 70-100% sequence identity to a sequence set forth in any one of SEQ ID NOs:51-69, for example, about: 75-100%, 75-99%, 80-100%, 80-98%, 85-100%, 85-97%, 90-100%, 90-96%, 95-100%, 96-100%, 97-100%, 98-100% or 99-100%. In some embodiments, the polynucleotide (e.g., ASO) comprises a nucleotide sequence that is identical to a sequence set forth in any one of SEQ ID NOs:51-69.

(W-704)
(SEQ ID NO: 51)
AGAAGCCAAAGGAGACCUGA.
(W-705)
(SEQ ID NO: 52)
AAAGAGAAGCCAAAGGAGAC.
(W-706)
(SEQ ID NO: 53)
CUAGACCGGAAAAGAGAAGCCA.
(W-707)
(SEQ ID NO: 54)
AUGCUAGACCGGAAAAGAGAA.
(W-708)
(SEQ ID NO: 55)
CAAUGCUAGACCGGAAAAGA.
(W-709)
(SEQ ID NO: 56)
AAGUCCCAAUGCUAGACCGGA.
(W-710)
(SEQ ID NO: 57)
UCUCCGAAGUCCCAAUGCUA.
(W-711)
(SEQ ID NO: 58)
GAGCUCUCCGAAGUCCCA.
(W-712)
(SEQ ID NO: 59)
AGAACAGUGGAGCUCUCCGA.
(W-713)
(SEQ ID NO: 60)
CGCCCAGAACAGUGGAGCUC.
(W-714)
(SEQ ID NO: 61)
CCUCGCCCAGAACAGUGGAG.
(2831)
(SEQ ID NO: 62)
CAGUGGAGCUCUCCGAAGUCC.
(2832)
(SEQ ID NO: 63)
CCCAGAACAGUGGAGCUCUCC.
(2833)
(SEQ ID NO: 64)
CACAGCCCUCGCCCAGAACA.
(2834)
(SEQ ID NO: 65)
UUCUUCACAGCCCUCGCCCA.
(SEQ ID NO: 66)
UCUUUCUUCACAGCCCUCGCCCAGAACAGUGGAGCUCUCCGAAGUCCCAA
UGCUAGACCGGAAAAGAGAAGCCAAAGGAGACCUGA.
(SEQ ID NO: 67)
UCUCCGAAGUCCCAAUGCUAGACCGGAAAAGAGAAGCCAAAGGAGACCUG
A.
(SEQ ID NO: 68)
UCUUUCUUCACAGCCCUCGCCCAGAACAGUGGAGCUCUCCGAAGUCCCAA
UG.
(SEQ ID NO: 69)
UUCUUCACAGCCCUCGCCCAGAACAGUGGAGCUCUCCGAAGUCCCA.

In some embodiments, a polynucleotide disclosed herein (e.g., ASO) comprises a nucleotide sequence having at least 70% sequence identity to, for example, at least: 75%, 80%, 85%, 90%, 95%, 96%, 97%, 98% or 99% sequence identity to a sequence set forth in any one of SEQ ID NOs:60-65. In certain embodiments, the polynucleotide (e.g., ASO) comprises a nucleotide sequence having about: 70%, 75%, 80%, 85%, 90%, 95%, 96%, 97%, 98% or 99% sequence identity to a sequence set forth in any one of SEQ ID NOs:60-65. In some embodiments, the polynucleotide (e.g., ASO) has about: 70%, 75%, 80%, 85%, 90%, 95%, 96%, 97%, 98% or 99% sequence identity to a sequence set forth in any one of SEQ ID NOs:60-65. In some embodiments, the polynucleotide (e.g., ASO) comprises a nucleotide sequence having about 70-100% sequence identity to a sequence set forth in any one of SEQ ID NOs:60-65, for example, about: 75-100%, 75-99%, 80-100%, 80-98%, 85-100%, 85-97%, 90-100%, 90-96%, 95-100%, 96-100%, 97-100%, 98-100% or 99-100%. In some embodiments, the polynucleotide (e.g., ASO) comprises a nucleotide sequence that is identical to a sequence set forth in any one of SEQ ID NOs:60-65.

In some embodiments, an agent disclosed herein comprises a first polynucleotide (e.g., ASO) comprising a nucleotide sequence having at least 70% sequence identity, for example, at least: 75%, 80%, 85%, 90%, 95%, 96%, 97%, 98% or 99% sequence identity, to SEQ ID NO:60, and a second polynucleotide (e.g., ASO) comprising a nucleotide sequence having at least 70% sequence identity, for example, at least: 75%, 80%, 85%, 90%, 95%, 96%, 97%, 98% or 99% sequence identity. to SEQ ID NO:61. In some embodiments, the first polynucleotide (e.g., ASO) comprises a nucleotide sequence having about: 70%, 75%, 80%, 85%, 90%, 95%, 96%, 97%, 98% or 99% sequence identity to SEQ ID NO:60, and the second polynucleotide (e.g., ASO) comprises a nucleotide sequence having about: 70%, 75%, 80%, 85%, 90%, 95%, 96%, 97%, 98% or 99% sequence identity to SEQ ID NO:61. In some embodiments, the first polynucleotide (e.g., ASO) comprises a nucleotide sequence having about 70-100% sequence identity to SEQ ID NO:60, for example, about: 75-100%, 75-99%, 80-100%, 80-98%, 85-100%, 85-97%, 90-100%, 90-96%, 95-100%, 96-100%, 97-100%, 98-100% or 99-100%, sequence identity to SEQ ID NO:60; and the second polynucleotide (e.g., ASO) comprises a nucleotide sequence having about 70-100% sequence identity to SEQ ID NO:61, for example, about: 75-100%, 75-99%, 80-100%, 80-98%, 85-100%, 85-97%, 90-100%, 90-96%, 95-100%, 96-100%, 97-100%, 98-100% or 99-100% sequence identity to SEQ ID NO:61. In some embodiments, the first polynucleotide (e.g., ASO) comprises a nucleotide sequence that is identical to SEQ ID NO:60, and the second polynucleotide comprises a nucleotide sequence that is identical to SEQ ID NO:61.

In some embodiments, the polynucleotide (e.g., ASO) comprises a nucleotide sequence that is at least about 70% identical to a sequence within X chromosome region between 147,912,230 and 147,914,451 (e.g., between 147,912,230 and 147,912,728 or between 147,912,728 and 147,914,451), for example, at least about: 75%, 80%, 85%, 90%, 95%, 96%, 97%, 98%, 99% or 100% identical to the sequence within X chromosome region between 147,912,230 and 147,912,728. In some embodiments, the polynucleotide comprises a nucleotide sequence that is about: 70%, 75%, 80%, 85%, 90%, 95%, 96%, 97%, 98%, 99% or 100% identical to a sequence within X chromosome region between 147,912,230 and 147,914,451 (e.g., between 147,912,230 and 147,912,728 or between 147,912,728 and 147,914,451). In some embodiments, the polynucleotide comprises a nucleotide sequence having about 70%, 75%, 80%, 85%, 90%, 95%, 96%, 97%, 98%, 99% or 100% sequence identity to a sequence within X chromosome region between 147,912,230 and 147,914,451 (e.g., between 147,912,230 and 147,912,728 or between 147,912,728 and 147,914,451). In some embodiments, the polynucleotide comprises a nucleotide sequence having about 70-100% sequence identity to a sequence within X chromosome region between 147,912,230 and 147,914,451 (e.g., between 147,912,230 and 147,912,728 or between 147,912,728 and 147,914,451), for example, about: 75-100%, 75-99%, 80-100%, 80-98%, 85-100%, 85-97%, 90-100%, 90-96%, 95-100%, 96-100%, 97-100%, 98-100% or 99-100%.

In some embodiments, the polynucleotide (e.g., ASO) is at least about 70% complimentary to at least a portion of an FMR1 gene transcript, for example, at least about: 75%, 80%, 85%, 90%, 95%, 96%, 97%, 98%, 99% or 100% complimentary to at least a portion of an FMR1 gene transcript. In some embodiments, the polynucleotide is about: 70%, 75%, 80%, 85%, 90%, 95%, 96%, 97%, 98%, 99% or 100% complimentary to at least a portion of an FMR1 gene transcript. In some embodiments, the polynucleotide is about 70-100% complimentary to at least a portion of an FMR1 gene transcript, for example, about: 75-100%, 75-99%, 80-100%, 80-98%, 85-100%, 85-97%, 90-100%, 90-96%, 95-100%, 96-100%, 97-100%, 98-100% or 99-100% complimentary to at least a portion of an FMR1 gene transcript.

In some embodiments, a polynucleotide disclosed herein has a length of at least about 8 nucleotides, for example, at least about: 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 or 80 nucleotides. In some embodiments, the polynucleotide has a length of about 8-80 nucleotides, for example, about: 10-60, 10-40, 12-80, 12-60, 12-40, 12-38, 12-30, 13-38, 13-36, 14-36, 14-34, 15-80, 15-60, 15-40, 15-34, 15-32, 16-32, 16-30, 17-30, 17-28, 18-28, 18-26, 19-26, 19-24, 20-80, 20-60, 20-40, 20-30, 20-24 or 20-22 nucleotides. In some embodiments, the polynucleotide has a length of about 10-30 or 12-30 nucleotides. In some embodiments, the polynucleotide has a length of about: 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 or 80 nucleotides.

In some embodiments, a polynucleotide disclosed herein has a length of at least about 12 nucleotides, for example, at least about: 13, 14, 15, 16, 17, 18, 19, 20, 21, 22, 23, 24, 25, 26, 27, 28, 29 or 30 nucleotides. In some embodiments, the polynucleotide has a length of about 12-40 nucleotides, for example, about: 12-35, 12-30, 12-25, 13-40, 13-35, 13-30, 13-25, 14-40, 14-35, 14-30, 14-25, 15-40, 15-35, 15-30 or 15-25 nucleotides. In some embodiments, the polynucleotide has a length of about 15-25 nucleotides. In some embodiments, the polynucleotide has a length of about: 12, 13, 14, 15, 16, 17, 18, 19, 20, 21, 22, 23, 24, 25, 26, 27, 28, 29, 30, 35 or 40 nucleotides. In some embodiments, a polynucleotide is an oligonucleotide. In some embodiments, the length of the polynucleotide is about 18-22 nucleotides.

In some embodiments, a polynucleotide disclosed herein (e.g., oligonucleotide) is an isolated polynucleotide. An “isolated polynucleotide” refers to a polynucleotide that has been separated from other cellular components normally associated with native nucleotide polymers, including proteins and other nucleotide sequences. In some embodiments, the polynucleotide is an isolated DNA polynucleotide. In some embodiments, the polynucleotide is an isolated RNA polynucleotide.

Polynucleotides of the disclosure can be produced recombinantly or synthetically, using methods, techniques and reagents that are well known in the art, such as routine and well known molecular cloning techniques and solid-phase synthesis techniques. In some embodiments, a polynucleotide of the disclosure is a recombinant polynucleotide.

In another aspect, the present disclosure provides a polynucleotide capable of increasing the expression of a functional FMR1 gene product. The polynucleotide is any one of the polynucleotides, modified or unmodified, disclosed herein. In some embodiments, the polynucleotide is any one of the modified polynucleotides disclosed herein.

### Modification of Polynucleotides

In some embodiments, a polynucleotide of the disclosure comprises one or more modified nucleotides. In some embodiments, one or more modified nucleotides each independently comprises a modification of a ribose or deoxyribose group, a phosphate group, a nucleobase, or a combination thereof.

Chemical modifications can be chosen to, e.g., increase nuclease resistance of a polynucleotide (e.g., oligonucleotide), to prevent RNase H cleavage of a polynucleotide (e.g., a complementary RNA strand), or to increase cellular uptake of a polynucleotide. For each of these goals, a variety of compatible chemical modifications are available and will be familiar to those skilled in the art.

In some embodiments, a ribose or deoxyribose group comprises 2′-O-methyl, 2′-fluoro, 2′-deoxy, 2′-O-methoxyethyl (MOE; also 2′-O-(2-methoxyethyl)), 2′-O-alkyl, 2′-O-alkoxy, 2′-O-alkylamino, or 2′-NH₂ modification, a constrained nucleotide, a tricyclo-DNA modification, or a combination thereof.

In some embodiments, a substituted RNA analogue disclosed herein comprises a methoxyethyl group on the 2′OH.

In some embodiments, a constrained nucleotide comprises a locked nucleic acid (LNA), an ethyl-constrained nucleotide, a 2′-(S)-constrained ethyl (S-cEt) nucleotide, a constrained MOE, a 2′-0,4′-C-aminomethylene bridged nucleic acid (2′,4′-BNANC), an alpha-L-locked nucleic acid, and a tricyclo-DNA, or a combination thereof.

In some embodiments, modification of a ribose or deoxyribose group comprises a 2′-O-(2-methoxyethyl) (MOE) modification. In some embodiments, every nucleotide of a polynucleotide (e.g., oligonucleotide) comprises a 2′-O-(2-methoxyethyl) (MOE) modification.

In some embodiments, modification of a ribose or deoxyribose group comprises a tricyclo-DNA modification. In some embodiments, every nucleotide of a polynucleotide (e.g., antisense oligonucleotide) comprises a tricyclo-DNA modification.

In some embodiments, modification of a ribose group comprises a 2′-deoxy modification.

In some embodiments, each modification of a phosphate group comprises a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof. In some embodiments, each modification of a phosphate group comprises a phosphoramidate.

In some embodiments, modification of a phosphate group comprises a phosphorothioate modification. In some embodiments, every nucleotide of a polynucleotide (e.g., oligonucleotide) comprises a phosphorothioate modification. In some embodiments, a polynucleotide is a phosphorothioate-modified polynucleotide.

In some embodiments, a sugar-phosphate backbone is replaced with a phosphorodiamidate morpholino (PMO) backbone. In other embodiments, a sugar-phosphate backbone is replaced with a peptide nucleic acid or other pseudopeptide backbone.

In some embodiments, a polynucleotide backbone comprises a sugar phosphate backbone, a phosphorodiamidate mopholino (PMO) backbone, a peptide nucleic acid backbone, a pseudopeptide backbone, or a combination thereof.

In some embodiments, each modification of a nucleobase comprises 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, halogenated aromatic groups, or a combination thereof.

In some embodiments, modification of a nucleobase group comprises a 5-methylcytosine modification.

In some embodiments, a polynucleotide comprises a mixture of modified nucleotides.

In some embodiments, a mixture of modified nucleotides comprise two or more modifications selected from the group consisting of: 2′-O-methyl, 2′-deoxy, 2′-O-(2-methoxyethyl) (MOE), LNA, and tricyclo-DNA.

In some embodiments, a polynucleotide comprises 4 or fewer consecutive 2′-deoxy modified nucleotides.

In some embodiments, a mixture of modified nucleotides comprise one or more 2′-O-methyl modified nucleotides and one or more LNA modified nucleotides.

In some embodiments, a mixture of modified nucleotides comprises one or more 2′-O-(2-methoxyethyl) (MOE) modified nucleotides and one or more LNA modified nucleotides.

In some embodiments, each ribose or deoxyribose group of a polynucleotide disclosed herein (e.g., ASO) comprises 2′-O-(2-methoxyethyl) (MOE) and/or each phosphate group of the polynucleotide comprises a phosphorothioate. In some embodiments, each ribose or deoxyribose group of the polynucleotide (e.g., ASO) comprises 2′-O-(2-methoxyethyl) (MOE). In some embodiments, each phosphate group of the polynucleotide comprises a phosphorothioate. In some embodiments, each ribose or deoxyribose group of a polynucleotide disclosed herein (e.g., ASO) comprises 2′-O-(2-methoxyethyl) (MOE), and each phosphate group of the polynucleotide comprises a phosphorothioate.

### Polypeptides

In some embodiments, an agent disclosed herein comprises a polypeptide. As used herein, the term “polypeptide” refers to a polymer of at least two amino acids covalently linked by an amide bond, regardless of length or post-translational modification (e.g., glycosylation or phosphorylation). A polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a polypeptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a polypeptide can also be derivatized (e.g., alkylated) or labeled (e.g., with a detectable label, such as a fluorogen or a hapten) using methods known in the art. A polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a polypeptide can be an analog of a known and/or naturally-occurring peptide, for example, a peptide analog having conservative amino acid residue substitution(s).

In some embodiments, a polypeptide disclosed herein is an isolated polypeptide. In some embodiments, a polypeptide disclosed herein is a recombinant polypeptide.

In some embodiments, the polypeptide is an inhibitor (e.g., a direct inhibitor or an indirect inhibitor) of expression of an aberrant FMR1 gene product (e.g., FMR1-217, and/or its protein product). In some embodiments, the polypeptide is an activator (e.g., a direct activator or an indirect activator) of expression of a normal FMR1 gene product (e.g., FMR1-205, and/or its protein product). In some embodiments, the polypeptide reduces expression of an aberrant FMR1 gene product (e.g., FMR1-217, and/or its protein product) and increases expression of a normal FMR1 gene product (e.g., FMR1-205, and/or its protein product).

In some embodiments, a polypeptide disclosed herein is an immunoglobulin molecule. In some embodiments, the immunoglobulin molecule an antibody. In some embodiments, the antibody is an antagonist antibody that binds an FMR1 transcript, or isoform, associated with a fragile X-associated disorder (e.g., FXS). The antibody can be of any species, such as a rodent (e.g., murine, rat, guinea pig) antibody, a primate (e.g., human) antibody, or a chimeric antibody. In some embodiments, the antibody is primatized (e.g., humanized). In some embodiments, the antibody is a polyclonal antibody. In some embodiments, the antibody is a monoclonal antibody. In some embodiments, the antibody (e.g., monoclonal antibody) is multispecific, e.g., bi-, tri-, or quad-specific.

In some embodiments, a polypeptide disclosed herein is an antigen-binding fragment of an immunoglobulin molecule (e.g., an antibody), that retains the antigen binding properties of the parental full-length immunoglobulin molecule. In some embodiments, the antigen-binding fragment is a Fab, Fab′, F(ab′)2, Fd, Fv, disulfide-linked Fvs (sdFv, e.g., diabody, triabody or tetrabody), scFv, SMIP or rlgG.

In some embodiments, a polypeptide disclosed herein is an antibody mimetic. The term “antibody mimetic” refers to polypeptides capable of mimicking an antibody's ability to bind an antigen, but structurally differ from native antibody structures. Examples of antibody mimetics include, but not limited to, Adnectins, Affibodies, Affilins, Affimers, Affitins, Alphabodies, Anticalins, Avimers, DARPins, Fynomers, Kunitz domain peptides, monobodies, nanobodies, nanoCLAMPs, and Versabodies.

Techniques, assays and reagents for making and using therapeutic antibodies, or antigen-binding fragments thereof, against a target antigen (e.g., an FMR1 transcript, or isoform, associated with a fragile X-associated disorder, such as FXS) are known in the art. See, e.g., Therapeutic Monoclonal Antibodies: From Bench to Clinic (Zhiqiang An eds., 1 st ed. 2009); Antibodies: A Laboratory Manual (Edward A. Greenfield eds., 2d ed. 2013); Ferrara et al., Using Phage and Yeast Display to Select Hundreds of Monoclonal Antibodies: Application to Antigen 85, a Tuberculosis Biomarker, PLoS ONE 7(11): e49535 (2012), for techniques and methods of screening, making, purifying, storing, labeling, and characterizing antibodies.

### Gene Editing Systems

In some embodiments, an agent disclosed herein comprises a gene editing system. In some embodiments, the gene editing system produces a deletion of nucleotides, a substitution of nucleotides, an addition of nucleotides or a combination of the foregoing, in the FMR1 gene. In some embodiments, the gene editing system produces a partial or complete deletion in Exon 2 of FMR1-217 (e.g., pseudo exon between base pairs 147,911,919 and 147,914,451 in the human FMR1 gene).

In some embodiments, the gene editing system is a CRISPR/Cas system, a transposon-based gene editing system, or a transcription activator-like effector nuclease (TALEN) system. In some embodiments, the gene editing system is a CRISPR/Cas system. In some embodiments, the gene editing system is a class II CRISPR/Cas system.

In some embodiments, the gene editing system comprises a single Cas endonuclease or a polynucleotide encoding the single Cas endonuclease. In some embodiments, the single Cas endonuclease is Cas9, Cpf1, C2C1 or C2C3. In some embodiments, the single Cas endonuclease is Cas9 (e.g., of Streptococcus Pyogenes). In some embodiments, the single Cas endonuclease is Cpf1. In some embodiments, the Cpf1 is AsCpf1 (from Acidaminococcus sp.) or LbCpf1 (from Lachnospiraceae sp.). The choice of nuclease and gRNA(s) will typically be determined according to whether a deletion, a substitution, or an addition of nucleotide(s) to a targeted sequence is desired.

In some embodiments, the type II Cas endonuclease is Cas 9 (e.g., of Streptococcus pyogenes). In some embodiments, the modified Cas 9 is nickase Cas9, dead Cas9 (dCas9) or eSpCas9. In some embodiments, the nickase Cas9 is Cas9 D10A. In some embodiments, the dCas9 is D10A or H840A. In some embodiments, the gene editing system comprises a double nickase Cas9 (e.g., to achieve more accurate genome editing, see, e.g., Ran et al., Cell 154: 1380-89 (2013). Wild-type Cas9 generates double-strand breaks (DSBs) at specific DNA sequences targeted by a gRNA. Nickase Cas9 generates only a single-strand break. dCas9 is catalytically inactive. In some embodiments, dCas9 is fused to a nuclease (e.g., a FokI to generate DSBs at target sequences homologous to two gRNAs). Various CRISPR/Cas9 plasmids are publicly available from the Addgene repository (Addgene, Cambridge, MA: addgene.org/crispr/).

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. CRISPR technology for generating mtDNA dysfunction in the mitochondrial genome is disclosed in Jo et al., BioMed Res. Int. 2015: 305716 (2015). Co-delivery of Cas9 and sgRNA with nanoparticles is disclosed in Mout et al., ACS Nano 11(3): 2452-58 (2017).

In some embodiments, the agent comprises a small molecule. In some embodiments, the small molecule binds to a protein capable of modulating the splicing and/or expression of FMR1 or a fragment thereof. In some embodiments, the small molecule is an inhibitor of the target protein (e.g., a direct inhibitor, an indirect inhibitor). In some embodiments, the small molecule is an activator of the target protein (e.g., a direct activator, and indirect activator). Non-limiting examples of small molecules include organic compounds, organometallic compounds, inorganic compounds, and salts of organic, organometallic or inorganic compounds.

### Subjects

The term “subject” refers to a mammalian subject, preferably human, diagnosed with or suspected of having a fragile X-associated disorder (e.g., FXS).

In some embodiments, the subject comprises a CGG repeat expansion between about 55 and about 200 repeats in the 5′ untranslated region of an FMR1 gene. In some embodiments, the subject comprises a CGG repeat expansion exceeding 200 repeats in the 5′ untranslated region of an FMR1 gene. In some embodiments, the subject comprises a CGG repeat expansion that is partially methylated. In some embodiments, the subject comprises a CGG repeat expansion that is fully methylated. In some embodiments, the subject has an increased level of isoform 12 of FMR1, a decreased level of isoform 1 of FMR1, or a combination thereof.

In some embodiments, the subject has one X chromosome and one Y chromosome. In some embodiments, the subject has two X chromosomes. In some embodiments, the subject has two X chromosomes and one Y chromosome. In some embodiments, the subject has one X chromosome and two Y chromosomes.

In some embodiments, the subject is a human male. In some embodiments the subject is human female.

In some embodiments, the subject is at least about 1 month of age, for example, at least about 2, 3, 4, 5, 6, 7, 8, 9, 10, 11, 12, 15, 18 or 21 months of age, or at least about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 years of age. In some embodiments, the subject is about: 1-100, 1-80, 1-60, 1-30, 1-24, 1-20, 1-18, 1-12, 1-10, 1-8, 1-6, 2-100, 2-80, 2-60, 2-30, 2-24, 2-20, 2-18, 2-12, 2-10, 2-8, 2-6, 3-100, 3-80, 3-60, 3-30, 3-24, 3-20, 3-18, 3-12, 3-10, 3-8, 3-6, 4-100, 4-80, 4-60, 4-30, 4-24, 4-20, 4-18, 4-12, 4-10, 4-8, 4-6, 5-100, 5-80, 5-60, 5-30, 5-24, 5-20, 5-18, 5-12, 5-10, 5-8, 6-100, 6-80, 6-60, 6-30, 6-24, 6-20, 6-18, 6-12, 6-10, 8-100, 8-80, 8-60, 8-30, 8-24, 8-20, 8-18, 8-12, 10-100, 10-80, 10-60, 10-30, 10-24, 10-20, 10-18, 12-100, 12-80, 12-38, 12-60, 12-50, 12-40, 12-30, 12-24, 12-20, 12-18, 18-100, 18-80, 18-60, 18-50, 18-40, 18-30, 18-24, 20-100, 20-80, 20-60, 20-50, 20-40, 20-30, 20-25, 30-100, 30-80, 30-60, 30-55, 30-50, 30-45, 30-40, 40-100, 40-80, 40-60, 40-55 or 40-50 years of age. In some embodiments, the subject is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 or 100 years of age. In some embodiments, the subject is about 12-38 years of age. In other embodiments, the subject is a fetus. In some embodiments, the subject is a neonatal subject.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older, 75 years of age or older or 90 years of age or older. In some embodiments, the subject is 50 years of age or older. In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject is about 2-11, 4-17, 12-18, 18-50, 18-90 or 50-90 years of age.

In some embodiments, a subject is a human. In some embodiments, the human subject has, or is predisposed to have a fragile X-associated disorder. In some embodiments the human subject has, or is predisposed to have, FXS, FXPOI, FXTAS, or a combination thereof. In some embodiments, the human subject has, or is predisposed to have FXS. In some embodiments, the subject is a human (e.g., about 50 years of age or older) who has, or is predisposed to have, FXTAS.

In some embodiments, the subject has one or more of the physical and/or medical features associated with a fragile X-associated disorder (e.g., FXS). Non-limiting examples of physical features associated with FXS include a long face, prominent ears and chin, arched palate, large testicles at puberty, low muscle tone, flat feet, and hyperextensible joints. Non-limiting examples of medical or behavioral features associated with FXS include sleep problems, seizures, recurrent ear infections, mitral valve prolapse, behaviors of hyperactivity, short attention span, hand biting or hand flapping, poor eye contact and social skills, shyness, anxiety, autism, epilepsy, aggression, delayed speech and/or motor development, repetitive speech, sensitivity to sensory stimulation (including a hypersensitivity to being touched, to light or to sound), or any combination thereof. In some embodiments, the subject is a female with an intelligence quotient (IQ) score of less than 115, 110, 105, 100, 95 or 90. In some embodiments, the subject is a male with an IQ score of less than 60, 55, 50 or 45.

In some embodiments, the subject has one or more of the following: irregular menses, fertility problem, elevated FSH (follicle-stimulating hormone) level, premature ovarian failure, primary ovarian insufficiency, and vasomotor symptoms (e.g., “hot flash”). In some embodiments, the subject has one or more of the following: intention tremor, parkinsonism, ataxia, memory loss, white matter lesion involving middle cerebellar peduncles, and cognitive decline.

### Treatments

“Treat,” “treating” or “treatment” refers to therapeutic treatment wherein the objective is to slow down (lessen) an undesired physiological change or disease, such as the development or progression of the fragile X-associated disorder (e.g., FXS), or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, whether detectable or undetectable.

In some embodiments, the method further comprises assessing the efficacy of the agent (e.g., polynucleotide such as ASO) (outcome measure) for treatment of the fragile X-associated disorder (e.g., FXS) in the subject, comprising assaying a biological sample from the subject for the presence and/or level of FMR1 RNA isoform 1, FMR1 RNA isoform 12, or a combination thereof.

In some embodiments, treating a fragile X-associated disorder (e.g., FXS) includes slowing progression of the fragile X-associated disorder (e.g., FXS), alleviating one or more signs or symptoms of the fragile X-associated disorder (e.g., FXS), preventing one or more signs or symptoms of the fragile X-associated disorder (e.g., FXS), or a combination thereof.

Non-limiting examples of treatment benefits include improvements in speech and motor development; a reduction in or prevention of cognitive disabilities, ranging from learning disabilities to intellectual disability; alleviating or preventing physical and medical features such as a long face, prominent ears and chin, arched palate, large testicles at puberty, low muscle tone, flat feet, hyperextensible joints, sleep problems, seizures, recurrent ear infections, and mitral valve prolapse; reducing or preventing behaviors of hyperactivity, short attention span, hand biting or hand flapping, poor eye contact and social skills, shyness, anxiety, delayed speech and/or motor development, repetitive speech, and/or sensitivity to sensory stimulation (including a hypersensitivity to being touched).

In some embodiments, treatment may include modulation of or improvement in language, fragile X behaviors, brain activity, clinical impression, inattention, safety, social avoidance, cognition, hyperactivity, executive function, irritability, eye contact, or memory.

In some embodiments, treatment results in an intelligence quotient (IQ) score of at least about 40, for example, at least about: 45, 50, 55, 60, 65, 70, 75, 80, 85, 90, 95, 100, 105, 110, 115, 120, 125, or 130. In some embodiments, treatment results in an IQ score between about: 40-110, 40-100, 50-105, 60-80, 65-90, 70-80, 75-95, or 70-100. In some embodiments, treatment results in an IQ score of about 40, 45, 50, 55, 60, 65, 70, 75, 80, 85, 90, 95, 100, 105, 110, 115, 120, 125, or 130. In some embodiments, treatment results in an increase in IQ score of at least about: 1, 2, 3, 4, 5, 6, 7, 8, 9, 10, 11, 12, 13, 14, 15, 16, 17, 18, 19, or 20 points. In some embodiments, treatment results in an increase in IQ score of between about: 1-10, 1-15, 2-20, 2-15, 2-10, 5-15, 5-10, 10-20, or 15-20 points. In some embodiments, treatment results in an increase in IQ score of about: 1, 2, 3, 4, 5, 6, 7, 8, 9, 10, 11, 12, 13, 14, 15, 16, 17, 18, 19, or 20 points.

In still other embodiments, treatment can include reducing or preventing absent or irregular menses, fertility problems, elevated FSH (follicle-stimulating hormone) levels, premature ovarian failure, primary ovarian insufficiency, and/or hot flashes. In still further embodiments, treating may include reducing or preventing intention tremors, parkinsonism, ataxia, memory loss, white matter lesions involving middle cerebellar peduncles, and/or cognitive decline. In some embodiments, treatment may reduce or prevent neuropathy of extremities, mood instability, irritability, explosive outbursts, personality changes, autonomic function problems such as impotence, loss of bladder or bowel functions. Treatment may also include reducing or preventing high blood pressure, thyroid disorders, or fibromyalgia.

### Formulation and Administration

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual.

In some embodiments, an agent disclosed herein (e.g., ASO) is in a form of a pharmaceutical composition, or a pharmaceutically acceptable salt thereof. A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a pharmaceutical composition disclosed herein is formulated as a solution.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., polynucleotide) is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

In some embodiments, a pharmaceutical composition suitable for use in methods disclosed herein further comprises one or more pharmaceutically acceptable carriers. The term “pharmaceutically acceptable carrier” refers to an ingredient in a pharmaceutical composition, other than an active ingredient, which is nontoxic to a subject and should not interfere with the efficacy of the active ingredient. A pharmaceutically acceptable carrier includes, but is not limited to, such as those widely employed in the art of drug manufacturing. The carrier may be a diluent, adjuvant, excipient, or vehicle with which the agent is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the agent in such pharmaceutical formulation may vary widely, e.g., from less than about 0.5%, usually to at least about 1% to as much as 15%, 20%, 25%, 30%, 35%, 40%, 45% or 50% by weight. The concentration will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

Agents (e.g., polynucleotides) disclosed herein may be prepared in accordance with standard procedures and are administered at dosages that are selected to reduce, prevent, or eliminate, or to slow or halt progression of, a condition being treated (See, e.g., Remington's Pharmaceutical Sciences, Mack Publishing Company, Easton, PA, and Goodman and Gilman's The Pharmaceutical Basis of Therapeutics, McGraw-Hill, New York, N.Y., the contents of which are incorporated herein by reference, for a general description of methods for administering various agents for human therapy).

In some embodiments, an agent disclosed herein (e.g., ASO) is delivered using controlled or sustained-release delivery systems (e.g., capsules, biodegradable matrices). Example delayed-release delivery systems for drug delivery that would be suitable for administration of a composition described herein are described in U.S. Pat. No. 5,990,092 (issued to Walsh); U.S. Pat. No. 5,039,660 (issued to Leonard); U.S. Pat. No. 4,452,775 (issued to Kent); and U.S. Pat. No. 3,854,480 (issued to Zaffaroni), the entire teachings of which are incorporated herein by reference.

For oral administration, polynucleotides may be in the form of, for example, a tablet, capsule, suspension or liquid. A polynucleotide is preferably made in the form of a dosage unit containing a therapeutically effective amount of an active ingredient. Examples of such dosage units are tablets and capsules. For therapeutic purposes, tablets and capsules can contain, in addition to an active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

Administration of the agent to the subject can be by parenteral or non-parenteral means. In some embodiments, an agent disclosed herein (e.g., ASO) is administered intravenously, intra-arterially, intrathecally, intraventricularly, intramuscularly, intradermally, subcutaneously, intracranially, or spinally. “Administering” or “administration” as used herein, refers to taking steps to deliver an agent to a subject, such as a mammal, in need thereof. Administering can be performed, for example, once, a plurality of times, and/or over one or more extended periods. Administration includes both direct administration, including self-administration, and indirect administration, including an act of prescribing a drug or directing a subject to consume an agent. For example, as used herein, one (e.g., a physician) who instructs a subject (e.g., a patient) to self-administer an agent (e.g., a drug), or to have an agent administered by another and/or who provides a patient with a prescription for a drug is administering an agent to a subject. Administration of an agent can be once in a day or more than once in a day (e.g., twice a day or more). Administration of the agent can be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose.

In some embodiments, an agent disclosed herein (e.g., polynucleotide such as ASO) is delivered locally to the central nervous system. This can include intrathecal or intraventricular injections, including the use of a catheter or Ommaya reservoir. Other methods of delivering agents (e.g., drugs) directly to the cerebrospinal fluid or central nervous system will be known to one skilled in the art.

In some embodiments, an agent disclosed herein (e.g., polynucleotide such as ASO) is administered as intrathecal bolus injection. In some embodiments, the agent (e.g., polynucleotide such as ASO) is administered at a dosage of about 4-20 mg per administration, for example, about: 4, 5, 6, 7, 8, 9, 10, 11, 12, 13, 14, 15, 16, 17, 18, 19 or 20 mg per administration. In some embodiments, the agent (e.g., polynucleotide such as ASO) is administered at a dosage of about 12 mg per administration. In some embodiments, the agent (e.g., polynucleotide such as ASO) is administered at a dosage of about, e.g., up to 50 or 100 mg per injection.

In some embodiments, an agent disclosed herein (e.g., polynucleotide such as ASO) is delivered systemically, such as via intravenous or subcutaneous injection. In some embodiments, the agent (e.g., polynucleotide such as ASO) is delivered using an approach that enhances bioavailability in the central nervous system after systemic administration. These approaches can include modification of the sugars or phosphate linkages, delivering as a duplex with a ligand-conjugated RNA molecule, formulation into an artificial exosome, liposome, polymer nanoparticle or lipid nanoparticle, or conjugation to lipids, antibodies, peptides, sugars, neuroactive molecules, or other moieties that enhance delivery to the central nervous system. In some embodiments, the agent (e.g., polynucleotide such as ASO) is delivered after transiently disrupting the blood-brain barrier. Other methods of enhancing bioavailability in the central nervous system after systemic administration will be known to one skilled in the art.

In some embodiments, a method disclosed herein comprises administering to the subject two or more polynucleotides, for example, 2, 3, 4, or 5 polynucleotides. In some embodiments, the two or more polynucleotides are administered together. In other embodiments, the two or more polynucleotides are administered separately.

In some embodiments, a first polynucleotide disclosed herein (e.g., ASO) comprises a nucleotide sequence having at least 70%, 75%, 80%, 85%, 90%, 95%, 96%, 97%, 98% or 99% sequence identity to a sequence set forth in any one of SEQ ID NOs:1-11, SEQ ID NOs:43-46, SEQ ID NOs:51-65. In some embodiments, the first polynucleotide comprises a nucleotide sequence having about: 70%, 75%, 80%, 85%, 90%, 95%, 96%, 97%, 98% or 99% sequence identity to a sequence set forth in any one of SEQ ID NOs:1-11, SEQ ID NOs:43-46, SEQ ID NOs:51-65. In some embodiments, the first polynucleotide comprises a nucleotide sequence set forth in any one of SEQ ID NOs:1-11, SEQ ID NOs:43-46, SEQ ID NOs:51-65.

In some embodiments, a second polynucleotide disclosed herein (e.g., ASO) comprises a nucleotide sequence having at least: 70%, 75%, 80%, 85%, 90%, 95%, 96%, 97%, 98% or 99% sequence identity to a sequence set forth in any one of SEQ ID NOs:1-11, SEQ ID NOs:43-46, SEQ ID NOs:51-65. In some embodiments, the second polynucleotide comprises a nucleotide sequence having about 70%, 75%, 80%, 85%, 90%, 95%, 96%, 97%, 98% or 99% sequence identity to a sequence set forth in any one of SEQ ID NOs:1-11, SEQ ID NOs:43-46, SEQ ID NOs:51-65. In some embodiments, the second polynucleotide comprises a nucleotide sequence set forth in any one of SEQ ID NOs:1-11, SEQ ID NOs:43-46, SEQ ID NOs:51-65.

In some embodiments, a method disclosed herein comprises administering to a subject a third, fourth, or fifth polynucleotide (e.g., ASO) comprising a nucleotide sequence having at least 70%, 75%, 80%, 85%, 90%, 95%, 96%, 97%, 98% or 99% sequence identity to a sequence set forth in any one of SEQ ID NOs:1-11, SEQ ID NOs:43-46, SEQ ID NOs:51-65. In some embodiments, the third, fourth, or fifth polynucleotide comprises a nucleotide sequence having about 70%, 75%, 80%, 85%, 90%, 95%, 96%, 97%, 98% or 99% sequence identity to a sequence set forth in any one of SEQ ID NOs:1-11, SEQ ID NOs:43-46, SEQ ID NOs:51-65. In still other embodiments, the third, fourth, or fifth polynucleotide comprises a nucleotide sequence set forth in any one of SEQ ID NOs:1-11, SEQ ID NOs:43-46, SEQ ID NOs:51-65.

In some embodiments, the method comprises administering to the subject an antisense oligonucleotide comprising a nucleotide sequence of SEQ ID NO:1, an antisense oligonucleotide comprising a nucleotide sequence of SEQ ID NO:2, or both. In some embodiments, the method comprises administering to the subject an antisense oligonucleotide comprising a nucleotide sequence of SEQ ID NO:6, an antisense oligonucleotide comprising a nucleotide sequence of SEQ ID NO:7, or both. In some embodiments, the method comprises administering to the subject an antisense oligonucleotide comprising a nucleotide sequence of SEQ ID NO:10, an antisense oligonucleotide comprising a nucleotide sequence of SEQ ID NO:11, or both.

In some embodiments, the method comprises administering to the subject an antisense oligonucleotide comprising a nucleotide sequence of SEQ ID NO:51, an antisense oligonucleotide comprising a nucleotide sequence of SEQ ID NO:52, or both. In some embodiments, the method comprises administering to the subject an antisense oligonucleotide comprising a nucleotide sequence of SEQ ID NO:56, an antisense oligonucleotide comprising a nucleotide sequence of SEQ ID NO:57, or both. In some embodiments, the method comprises administering to the subject an antisense oligonucleotide comprising a nucleotide sequence of SEQ ID NO:60, an antisense oligonucleotide comprising a nucleotide sequence of SEQ ID NO:61, or both.

In some embodiments, it may be advantageous to administer an agent (e.g., a polynucleotide such as an antisense oligonucleotide, a pharmaceutical composition thereof, or a pharmaceutically acceptable salt of the foregoing) of the present disclosure in combination with one or more additional therapeutic agent(s). For example, it may be advantageous to administer a compound of the present disclosure (e.g., an antisense oligonucleotide, or a pharmaceutical composition thereof, or a pharmaceutically acceptable salt of the foregoing) in combination with one or more additional therapeutic agents, e.g., a modulator of DNA methylation (e.g., an agent that inhibits DNA methylation or promotes DNA demethylation, see for example, the section of “DNA demethylation”) a metabotropic glutamate receptor 5 (mGluR5) modulators (e.g., Basimglurant or Mavoglurant), GABAB receptor activator (e.g., arbaclofen), GABAA or GABAB receptor activator (e.g., acamprosate), AMPAkine (e.g., AX516), CB1 inhibitor (e.g., rimonabant), RAS signaling inhibitor (e.g., lovastatin), STEP inhibitor, S6K inhibitor, PAK inhibitor (e.g., FRAX486), MMP9 inhibitor (e.g., minocycline), and GSK30 inhibitor (e.g., lithium). In some embodiments, treating the subject comprises providing the subject with a ketogenic (“keto”) diet.

The term “combination therapy” refers to the administration of two or more therapeutic agents to treat a disease, disorder or condition described herein. Such administration encompasses co-administration of the therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Such administration also encompasses use of each type of therapeutic agent in a sequential manner, either at approximately the same time or at different times. Therapeutic agents in a combination therapy can be administered via the same administration route or via different administration routes. Powders and/or liquids may be reconstituted or diluted to a desired dose prior to administration. Typically, the treatment regimen will provide beneficial effects of a drug combination in treating diseases, conditions or disorders described herein.

In some embodiments, a method of treatment disclosed herein further comprises administering to the subject a therapeutically effective amount of a DNA-demethylating compound or DNA demethylase, prior to, during, or after, administering an agent disclosed herein (e.g., polynucleotide such as an ASO). In some embodiments, the method of treatment further comprises administering to the subject a therapeutically effective amount of a DNA-demethylating compound or DNA demethylase after administering an agent disclosed herein (e.g., polynucleotide such as an ASO).

Non-limiting examples of DNA-demethylating compounds include 5-Azacytidine (5-Aza-CR) and 5-aza-2′-deoxycytidine (5-Aza-CdR), dihydro-5-azacytidine (DHAC), zebularine, 5-fluoro-2′-deoxycytidine, Hydralazine, RG108, procainamide, and SGI-1027. In some embodiments, the DNA-demethylating compound is a nucleoside analogue. In some embodiments, the DNA-demethylating compound is a non-nucleoside analogue.

In some embodiments, the DNA demethylase (e.g., DNA methylation modification enzymes Dnmt or Tet (dCas9-Dnmt/Tet) is fused to a catalytically inactivate Cas9. Under the guidance of a single guide RNA (sgRNA), the dCas9-Tet1 demethylates the FMR1 locus and promoter region when FMR1 has an expanded CGG repeat of 200 or more.

In some embodiments, the DNA-demethylating compound or DNA demethylase is in an amount sufficient to demethylate at least about 5% of an FMR1 gene, for example, at least about: 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the FMR1 gene. In some embodiments, the DNA-demethylating compound or DNA demethylase is in an amount sufficient to demethylate about: 10-100%, 10-90%, 15-90%, 15-80%, 15-75%, 20-75%, 20-70%, 25-60%, 25-55%, 25-50%, 30-40%, or 30-35% of an FMR1 gene. In some embodiments, a DNA demethylase is in an amount sufficient to demethylate about: 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of an FMR1 gene. In some embodiments, a DNA-demethylating compound or DNA demethylase is in an amount sufficient to demethylate about 25-50% of an FMR1 gene.

In some embodiments, a method of modulating FMR1 splicing and/or expression further comprising contacting the cell with a DNA-demethylating compound or DNA demethylase, prior to, during, or after, contacting the cell with the agent (e.g., polynucleotide). In some embodiments, a method of treatment disclosed herein further comprises decreasing (e.g., shortening or deleting) FMR1 CGG expansion (e.g., by CRISPR/Cas9 gene editing) in the subject, prior to, during, or after, administering an agent disclosed herein (e.g., polynucleotide such as an ASO). In some embodiments, the method of treatment further comprises decreasing (e.g., shortening or deleting) FMR1 CGG expansion prior to administering an agent disclosed herein (e.g., polynucleotide such as an ASO).

Methods of Modulating FMR1 Splicing and/or Expression

In another aspect, the present disclosure provides a method of modulating FMR1 splicing and/or expression in a cell, comprising contacting the cell with an agent (e.g., polynucleotide) under conditions whereby the agent is introduced into the cell, thereby modulates FMR1 splicing and/or expression in the cell. The agent can be any one of the agents disclosed herein.

In some embodiments, the agent (e.g., a polynucleotide of the disclosure, an agent that modulates DNA methylation, or a combination thereof) increases expression of isoform 1 of the FMR1 gene, increases splicing of isoform 1 (between X chromosome base pairs 147,912,230 and 147,921,933), decreases expression of isoform 12 of the FMR1 gene, decreases splicing of isoform 12 (between X chromosome between base pairs 147,912,230 and 147,912,728), or a combination thereof.

In some embodiments, the agent (e.g., a polynucleotide of the disclosure, an agent that modulates DNA methylation, or a combination thereof) increases the splicing and/or expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125% relative to the reference. In some embodiments, the agent (e.g., a polynucleotide of the disclosure, an agent that modulates DNA methylation, or a combination thereof) increases the splicing of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) increases the expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

In some embodiments, the agent (e.g., a polynucleotide of the disclosure, an agent that modulates DNA methylation, or a combination thereof) decreases the splicing and/or expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., polynucleotide) decreases the splicing of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) decreases the expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

In some embodiments, the agent (e.g., a polynucleotide of the disclosure, an agent that modulates DNA methylation, or a combination thereof) increases splicing and/or expression of isoform 1 of FMR1, decrease splicing and/or expression of isoform 12 of FMR1, or a combination thereof. “Isoform 1” or “iso1” refers to normal FMR1 RNA with exon 1 spliced to exon 2. “Isoform 12” or “iso12” refers to missplicing of FMR1 RNA, where exon 1 is spliced to a pseudo exon located within intron 1. Isoform 12 would generate a 31-amino acid protein, which probably would have no biological function. Note that iso-12 and FMR1-217 refer to the same FMR1 RNA isoform.

In some embodiments, the agent (e.g., a polynucleotide of the disclosure, an agent that modulates DNA methylation, or a combination thereof) increases isoform 1 of FMR1 by at least about 5% relative to a reference, e.g., by at least about: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125% relative to the reference. In some embodiments, the agent (e.g., polynucleotide) increases isoform 1 of the FMR1 gene by about 75%.

In some embodiments, the agent (e.g., a polynucleotide of the disclosure, an agent that modulates DNA methylation, or a combination thereof) decreases isoform 12 of FMR1 by at least about 5% relative to a reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) decreases isoform 12 of the FMR1 gene by about 30%.

In some embodiments, the level of splicing and/or expression of FMR1 or a fragment thereof, is measured after the agent is contacted with the cell for at least about 1 day, e.g., at least about: 2 days, 3 days, 4 days, 5 days, 6 days, 8 days, 9 days, 10 days, 1 week, 2 weeks, 3 weeks, 1 month, 2 months, 3 months, 4 months, 5 months or 6 months.

In some embodiments, the agent comprises, consists essentially of or consists of any one of the polypeptides, polynucleotides, gene editing systems or small molecules disclosed herein.

In some embodiments, the agent comprises at least one of the polynucleotides of the disclosure. In some embodiments, the agent comprises two or more of the polynucleotides of the disclosure.

In some embodiments, the cell is a fetal cell (e.g., circulating fetal cell), a blastomere, a trophectoderm cell, a stem cell (e.g., induced pluripotent stem cell (iPSC) or derived stem cell), a fibroblast, a modified fibroblast, a pluripotent cell, or a cultured cell.

In some embodiments, the cell is an in vitro cell or an ex vivo cell. In some embodiments, the cell is an iPSC-derived neuron from a human who has or is predisposed to have FXS, a primary human cell, or a cell line. In some embodiments, the cell is a cell of any one of the subjects disclosed herein. In some embodiments, the cell of the subject is allogeneic. In some embodiments, the cell of the subject is autologous or syngeneic.

### Methods of Reducing CGG Triplet Repeat Expansion in FMR1 5′ UTR

In another aspect, the present disclosure provides a method of reducing CGG triplet repeat expansion in FMR1 5′ UTR in a cell, comprising contacting the cell with an agent (e.g., a polynucleotide disclosed herein, an agent that modulates DNA methylation, or a combination thereof) under conditions whereby the agent is introduced into the cell, thereby reducing CGG triplet repeat expansion in the cell. The agent can be any one of the agents disclosed herein.

In some embodiments, the agent (e.g., a polynucleotide of the disclosure, an agent that modulates DNA methylation, or a combination thereof) increases expression of isoform 1 of the FMR1 gene, increases splicing of isoform 1 (between X chromosome between base pairs 147,912,230 and 147,921,933), decreases expression of isoform 12 of the FMR1 gene, decreases splicing of isoform 12 (between X chromosome between base pairs 147,912,230 and 147,912,728), or a combination thereof.

In some embodiments, the agent (e.g., a polynucleotide disclosed herein, an agent that modulates DNA methylation, or a combination thereof) increases the splicing and/or expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125% relative to the reference. In some embodiments, the agent (e.g., a polynucleotide of the disclosure, an agent that modulates DNA methylation, or a combination thereof) increases the splicing of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) increases the expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

In some embodiments, the agent (e.g., a polynucleotide of the disclosure, an agent that modulates DNA methylation, or a combination thereof) decreases the splicing and/or expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) decreases the splicing of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) decreases the expression of FMR1 or a fragment thereof, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

In some embodiments, the agent (e.g., a polynucleotide of the disclosure, an agent that modulates DNA methylation, or a combination thereof) increases splicing and/or expression of isoform 1 of FMR1, decrease splicing and/or expression of isoform 12 of FMR1, or a combination thereof. “Isoform 1” or “iso1” refers to normal FMR1 RNA with exon 1 spliced to exon 2. “Isoform 12” or “iso12” refers to missplicing of FMR1 RNA, where exon 1 is spliced to a pseudo exon located within intron 1. Isoform 12 would generate a 31-amino acid protein, which probably would have no biological function.

In some embodiments, the agent (e.g., a polynucleotide of the disclosure, an agent that modulates DNA methylation, or a combination thereof) increases isoform 1 of FMR1 by at least about 5% relative to a reference, e.g., by at least about: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125% relative to the reference. In some embodiments, the agent (e.g., a polynucleotide of the disclosure, an agent that modulates DNA methylation, or a combination thereof) increases isoform 1 of the FMR1 gene by about 75%.

In some embodiments, the agent (e.g., a polynucleotide of the disclosure, an agent that modulates DNA methylation, or a combination thereof) decreases isoform 12 of FMR1 by at least about 5% relative to a reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) decreases isoform 12 of the FMR1 gene by about 30%.

In some embodiments, the agent (e.g., a polynucleotide of the disclosure, an agent that modulates DNA methylation, or a combination thereof) decreases CGG triplet repeat expansion in FMR1 5′ UTR in the cell by at least about 5% relative to a reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In some embodiments, the agent (e.g., a polynucleotide of the disclosure, an agent that modulates DNA methylation, or a combination thereof) decreases CGG triplet repeat expansion in FMR1 5′ UTR in the cell by at least about 10%, relative to a reference.

In some embodiments, the level CGG triplet repeat in FMR1 5′ UTR in the cell, is measured after the agent is contacted with the cell for at least about 1 day, e.g., at least about: 2 days, 3 days, 4 days, 5 days, 6 days, 8 days, 9 days, 10 days, 1 week, 2 weeks, 3 weeks, 1 month, 2 months, 3 months, 4 months, 5 months or 6 months.

In some embodiments, the agent comprises, consists essentially of or consists of any one of the polypeptides, polynucleotides, gene editing systems or small molecules disclosed herein.

In some embodiments, the agent comprises at least one of the polynucleotides disclosed herein. In some embodiments, the agent comprises two or more of the polynucleotides disclosed herein.

In some embodiments, the cell is a fetal cell (e.g., circulating fetal cell), a blastomere, a trophectoderm cell, a stem cell (e.g., induced pluripotent stem cell (iPSC) or derived stem cell), a fibroblast, a modified fibroblast, a pluripotent cell, or a cultured cell.

In some embodiments, the cell is an in vitro cell or an ex vivo cell. In some embodiments, the cell is an iPSC-derived neuron from a human who has or is predisposed to have FXS, a primary human cell, or a cell line. In some embodiments, the cell is a cell of any one of the subjects disclosed herein. In some embodiments, the cell of the subject is allogeneic. In some embodiments, the cell of the subject is autologous or syngeneic.

In another aspect, the present disclosure provides a polynucleotide capable of reducing expression of an aberrant FMR1 gene product. The polynucleotide is any one of the polynucleotides, modified or unmodified, disclosed herein. In some embodiments, the polynucleotide is any one of the modified polynucleotides disclosed herein.

In another aspect, the present disclosure provides an agent that modulates splicing and/or expression of FMR1 gene. In some embodiments, the agent is a polynucleotide. In some embodiments, the agent is any one of the modified polynucleotides disclosed herein.

In yet another aspect, the present disclosure provides a pharmaceutical composition, comprising any one of the agents described herein, and one or more pharmaceutically acceptable excipients, diluents, or carriers.

In another aspect, the present disclosure provides an antisense oligonucleotide (ASO), wherein the ASO specifically binds a contiguous nucleotide sequence set forth in any one of SEQ ID NOs:24-42, and wherein the contiguous nucleotide sequence is at least 12 nucleotides in length.

In some embodiments, an ASO is at least: 12, 13, 14, 15, 16, 17, 18, 19, 20, 21, 22, 23, 24, 25, 26, 27, 28, 29, or 30 nucleotides in length. In some embodiments, an ASO is no more than: 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, or 18 nucleotides in length. In some embodiments, an ASO is about: 12-100, 12-35, 12-30, 12-25, 13-40, 13-35, 13-30, 13-25, 14-40, 14-35, 14-30, 14-25, 15-40, 15-35, 15-30, 15-25, 18-20, 18-21, 18-22, 18-23, 18-24, 19-20, 19-21, 19-22, 19-23, 19-24, 20-21, 20-22, 20-23, 20-24, 21-22, 21-23, 21-24, 22-23, 22-24, 23-24, 15-100, 15-90, 20-90, 20-80, 30-80, 30-70, 40-70, 40-60 or 50-60 nucleotides in length. In some embodiments, an ASO is about 18-24 nucleotides in length. In some embodiments, an ASO is about: 12, 13, 14, 15, 16, 17, 18, 19, 20, 21, 22, 23, 24, 25, 26, 27, 28, 29, 30, 35, 40, 50, 53, 58, 59, 60, 70, 80, 90, 93, 95, or 100 nucleotides in length.

In some embodiments, an ASO comprises:
at least one modification of a ribose or deoxyribose group,
at least one modification of a phosphate group,
at least one modification of a nucleobase,
or any combination of a) to c).

In some embodiments, an ASO comprises:
at least one modification of a ribose or deoxyribose group, and
at least one modification of a phosphate group.

In some embodiments, an ASO is modified to comprise:
a) a locked nucleic acid (LNA), an ethyl-constrained nucleotide, a 2′-(S)-constrained ethyl (S-cEt) nucleotide, a constrained 2′-O-methoxyethyl (MOE), a 2′-0,4′-C-aminomethylene bridged nucleic acid (2′,4′-BNA(NC)), an alpha-L-locked nucleic acid, a tricyclo-DNA, or a combination thereof,
b) a ribose or deoxyribose group comprising a 2′-O-methyl, 2′-fluoro, 2′-deoxy, 2′-O-methoxyethyl (MOE), 2′-O-alkyl, 2′-O-alkoxy, 2′-O-alkylamino, or 2′-NH₂ modification, a constrained nucleotide, a tricyclo-DNA modification, or a combination thereof,
c) a phosphate group comprising a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof,
d) a nucleobase comprising 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, halogenated aromatic groups, or a combination thereof,
e) a polynucleotide backbone comprising a sugar phosphate backbone, a phosphorodiamidate mopholino (PMO) backbone, a peptide nucleic acid backbone, a pseudopeptide backbone, or a combination thereof, or any combination of a) to e).

In some embodiments, an ASO comprises at least one phosphorothioate internucleotide linkage. In some embodiments, at least: 5%, 10%, 15%, 20%, 25%, 30%, 35%, 40%, 45%, 50%, 55%, 60%, 65%, 70%, 75%, 80%, 85%, 90%, or 95% of internucleotide linkages of an ASO are phosphorothioate internucleotide linkages. In some embodiments, 100% of internucleotide linkages of an ASO are phosphorothioate internucleotide linkages. In some embodiments, at most: 95%, 90%, 85%, 80%, 75%, 70%, 65%, 60%, 55%, 50%, 45%, 40%, 35%, 30%, 25%, 20%, 15%, 10%, or 5% of internucleotide linkages of an ASO are phosphorothioate internucleotide linkages. In some embodiments, about: 5-100%, 5-95%, 10-95%, 10-90%, 15-90%, 15-85%, 20-85%, 20-80%, 25-80%, 25-75%, 30-75%, 30-70%, 35-70%, 35-65%, 40-65%, 40-60%, 45-60%, 45-55%, or 50-55% of internucleotide linkages of an ASO are phosphorothioate internucleotide linkages. In some embodiments, about: 40-100%, 40-95%, 45-95%, 45-90%, 50-90%, 50-85%, 55-85%, 55-80%, 60-80%, 60-75%, 65-75%, or 65-70% of internucleotide linkages of an ASO are phosphorothioate internucleotide linkages.

In some embodiments, an ASO comprises at least one phosphodiester internucleotide linkage. In some embodiments, at least: 5%, 10%, 15%, 20%, 25%, 30%, 35%, 40%, 45%, 50%, 55%, 60%, 65%, 70%, 75%, 80%, 85%, 90%, or 95% of internucleotide linkages of an ASO are phosphodiester internucleotide linkages. In some embodiments, 100% of internucleotide linkages of an ASO are phosphodiester internucleotide linkages. In some embodiments, at most: 95%, 90%, 85%, 80%, 75%, 70%, 65%, 60%, 55%, 50%, 45%, 40%, 35%, 30%, 25%, 20%, 15%, 10%, or 5% of internucleotide linkages of an ASO are phosphodiester internucleotide linkages. In some embodiments, about: 5-100%, 5-95%, 10-95%, 10-90%, 15-90%, 15-85%, 20-85%, 20-80%, 25-80%, 25-75%, 30-75%, 30-70%, 35-70%, 35-65%, 40-65%, 40-60%, 45-60%, 45-55%, or 50-55% of internucleotide linkages of an ASO are phosphodiester internucleotide linkages. In some embodiments, about: 0-60%, 5-60%, 5-55%, 10-55%, 10-50%, 15-50%, 15-45%, 20-45%, 20-40%, 25-40%, 25-35%, or 30-35% of internucleotide linkages of an ASO are phosphodiester internucleotide linkages.

In some embodiments, an ASO comprises at least one 2′-O-methoxyethyl ribose sugar. In some embodiments, at least: 5%, 10%, 15%, 20%, 25%, 30%, 35%, 40%, 45%, 50%, 55%, 60%, 65%, 70%, 75%, 80%, 85%, 90%, or 95% of riboses or deoxyriboses of an ASO comprise a 2′-O-methoxyethyl ribose sugar. In some embodiments, 100% of riboses or deoxyriboses of an ASO comprise a 2′-O-methoxyethyl ribose sugar. In some embodiments, at most: 95%, 90%, 85%, 80%, 75%, 70%, 65%, 60%, 55%, 50%, 45%, 40%, 35%, 30%, 25%, 20%, 15%, 10%, or 5% of riboses or deoxyriboses of an ASO comprise a 2′-O-methoxyethyl ribose sugar. In some embodiments, about: 5-100%, 5-95%, 10-95%, 10-90%, 15-90%, 15-85%, 20-85%, 20-80%, 25-80%, 25-75%, 30-75%, 30-70%, 35-70%, 35-65%, 40-65%, 40-60%, 45-60%, 45-55%, or 50-55% of riboses or deoxyriboses of an ASO comprise a 2′-O-methoxyethyl ribose sugar. In some embodiments, about: 40-100%, 40-95%, 45-95%, 45-90%, 50-90%, 50-85%, 55-85%, 55-80%, 60-80%, 60-75%, 65-75%, or 65-70% of riboses or deoxyriboses of an ASO comprise a 2′-O-methoxyethyl ribose sugar.

In some embodiments, an ASO comprises a nucleotide sequence having at least 70% sequence identity to at least one sequence set forth in SEQ ID NOs:1-11, 43-50, and 51-75, for example, having at least: 75%, 80%, 85%, 88%, 90%, 92%, 95%, 96%, 97%, 98%, or 99% sequence identity to at least one sequence set forth in SEQ ID NOs:1-11, 43-50, and 51-75. In some embodiments, an ASO comprises a nucleotide sequence having at least 85% sequence identity to at least one sequence set forth in SEQ ID NOs:1-11, 43-50, and 51-75. In some embodiments, an ASO comprises a nucleotide sequence having 100% sequence identity to at least one sequence set forth in SEQ ID NOs:1-11, 43-50, and 51-75.

In some embodiments, an ASO comprises a nucleotide sequence having at least 70% sequence identity to at least one sequence set forth in SEQ ID NOs: 1-11, 43-46, 51-65, and 70-75, for example, having at least: 75%, 80%, 85%, 88%, 90%, 92%, 95%, 96%, 97%, 98%, or 99% sequence identity to at least one sequence set forth in SEQ ID NOs: 1-11, 43-46, 51-65, and 70-75. In some embodiments, an ASO comprises a nucleotide sequence having at least 85% sequence identity to at least one sequence set forth in SEQ ID NOs: 1-11, 43-46, 51-65, and 70-75. In some embodiments, an ASO comprises a nucleotide sequence having 100% sequence identity to at least one sequence set forth in SEQ ID NOs: 1-11, 43-46, 51-65, and 70-75.
CCTCGCCCAGAACAGTGGAGCTC (SEQ ID NO:70)
GCCCAGAACAGTGGAGCTC (SEQ ID NO:71)
CCTCGCCCAGAACAGTGGA (SEQ ID NO:72)
CCUCGCCCAGAACAGUGGAGCUC (SEQ ID NO:73)
GCCCAGAACAGUGGAGCUC (SEQ ID NO:74)
CCUCGCCCAGAACAGUGGA (SEQ ID NO:75)

In some embodiments, an ASO comprises a nucleotide sequence having at least 70% sequence identity to SEQ ID NO:10 or SEQ ID NO:60, for example, having at least: 75%, 80%, 85%, 88%, 90%, 92%, 95%, 96%, 97%, 98%, or 99% sequence identity to SEQ ID NO:10 or SEQ ID NO:60. In some embodiments, an ASO comprises a nucleotide sequence having at least 85% sequence identity to SEQ ID NO:10 or SEQ ID NO:60. In some embodiments, an ASO comprises a nucleotide sequence having 100% sequence identity to SEQ ID NO:10 or SEQ ID NO:60. In some embodiments, an ASO comprises:
(eC)#(eG)#(eC)#(eC)#(eC)#(eA)#(eG)#(eA)#(eA)#(eC)#(eA)#(eG)#(eT)#(eG)#(eG)#(e A)#(eG)#(eC)#(eT)#(eC),
(eC)#(eG)#(eC)#(eC)#(eC)#(eA)#(eG)#(eA)#(eA)#(eC)#(eA)#(eG)#(eU)#(eG)#(eG)#(e A)#(eG)#(eC)#(eU)#(eC),
(eC)#(eG)#(eC)#(eC)(eC)(eA)(eG)#(eA)#(eA)#(eC)#(eA)#(eG)#(eT)#(eG)(eG)(eA)(eG) #(eC)#(eT)#(eC), and/or
(eC)#(eG)#(eC)#(eC)(eC)(eA)(eG)#(eA)#(eA)#(eC)#(eA)#(eG)#(eU)#(eG)(eG)(eA)(eG) #(eC)#(eU)#(eC), wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:10, for example, having at least: 75%, 80%, 85%, 88%, 90%, 92%, 95%, 96%, 97%, 98%, or 99% sequence identity to SEQ ID NO:10. In some embodiments, an ASO comprises a nucleotide sequence having at least 85% sequence identity to SEQ ID NO:10. In some embodiments, an ASO comprises a nucleotide sequence having 100% sequence identity to SEQ ID NO:10.

In some embodiments, an ASO comprises:
(eC)#(eG)#(eC)#(eC)#(eC)#(eA)#(eG)#(eA)#(eA)#(eC)#(eA)#(eG)#(eT)#(eG)#(eG)#(e A)#(eG)#(eC)#(eT)#(eC), and/or
(eC)#(eG)#(eC)#(eC)(eC)(eA)(eG)#(eA)#(eA)#(eC)#(eA)#(eG)#(eT)#(eG)(eG)(eA)(eG) #(eC)#(eT)#(eC),
wherein e is a 2′-O-methoxyethyl ribose sugar, and wherein # is a phosphorothioate internucleotide linkage.

In some embodiments, an ASO comprises
(eC)#(eG)#(eC)#(eC)#(eC)#(eA)#(eG)#(eA)#(eA)#(eC)#(eA)#(eG)#(eT)#(eG)#(eG)#(eA)#(eG) #(eC)#(eT)#(eC), wherein e is a 2′-O-methoxyethyl ribose sugar, and wherein # is a phosphorothioate internucleotide linkage.

In some embodiments, an ASO comprises
(eC)#(eG)#(eC)#(eC)(eC)(eA)(eG)#(eA)#(eA)#(eC)#(eA)#(eG)#(eT)#(eG)(eG)(eA)(eG)#(eC)#(eT) #(eC), wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:60, for example, having at least: 75%, 80%, 85%, 88%, 90%, 92%, 95%, 96%, 97%, 98%, or 99% sequence identity to SEQ ID NO:60. In some embodiments, an ASO comprises a nucleotide sequence having at least 85% sequence identity to SEQ ID NO:60. In some embodiments, an ASO comprises a nucleotide sequence having 100% sequence identity to SEQ ID NO:60.

In some embodiments, an ASO comprises
(eC)#(eG)#(eC)#(eC)#(eC)#(eA)#(eG)#(eA)#(eA)#(eC)#(eA)#(eG)#(eU)#(eG)#(eG)#(e A)#(eG)#(eC)#(eU)#(eC), and/or
(eC)#(eG)#(eC)#(eC)(eC)(eA)(eG)#(eA)#(eA)#(eC)#(eA)#(eG)#(eU)#(eG)(eG)(eA)(eG) #(eC)#(eU)#(eC),
wherein e is a 2′-O-methoxyethyl ribose sugar, and wherein # is a phosphorothioate internucleotide linkage.

In some embodiments, an ASO comprises
(eC)#(eG)#(eC)#(eC)#(eC)#(eA)#(eG)#(eA)#(eA)#(eC)#(eA)#(eG)#(eU)#(eG)#(eG)#(eA)#(eG) #(eC)#(eU)#(eC), wherein e is a 2′-O-methoxyethyl ribose sugar, and wherein # is a phosphorothioate internucleotide linkage.

In some embodiments, an ASO comprises
(eC)#(eG)#(eC)#(eC)(eC)(eA)(eG)#(eA)#(eA)#(eC)#(eA)#(eG)#(eU)#(eG)(eG)(eA)(eG)#(eC) #(eU)#(eC), wherein e is a 2′-O-methoxyethyl ribose sugar, and wherein # is a phosphorothioate internucleotide linkage.

In some embodiments, an ASO comprises:
a nucleotide sequence having at least 70% sequence identity to SEQ ID NO:10 or SEQ ID NO:60, for example, having at least: 75%, 80%, 85%, 88%, 90%, 92%, 95%, 96%, 97%, 98%, or 99% sequence identity to SEQ ID NO:10 or SEQ ID NO:60; and
a nucleotide sequence having at least 70% sequence identity to SEQ ID NO:11 or SEQ ID NO:61, for example, having at least: 75%, 80%, 85%, 88%, 90%, 92%, 95%, 96%, 97%, 98%, or 99% sequence identity to SEQ ID NO:11 or SEQ ID NO:61.

In some embodiments, an ASO comprises:
a nucleotide sequence having at least 85% sequence identity to SEQ ID NO:10 or SEQ ID NO:60; and
a nucleotide sequence having at least 85% sequence identity to SEQ ID NO:11 or SEQ ID NO:61.

In some embodiments, an ASO comprises:
a nucleotide sequence having 100% sequence identity to SEQ ID NO:10 or SEQ ID NO:60; and
a nucleotide sequence having 100% sequence identity to SEQ ID NO:11 or SEQ ID NO:61.

In some embodiments, an ASO comprises:
(eC)#(eG)#(eC)#(eC)#(eC)#(eA)#(eG)#(eA)#(eA)#(eC)#(eA)#(eG)#(eT)#(eG)#(eG)#(e A)#(eG)#(eC)#(eT)#(eC) or (eC)#(eG)#(eC)#(eC)#(eC)#(eA)#(eG)#(eA)#(eA)#(eC)#(eA)#(eG)#(eU)#(eG)#(eG) #(eA)#(eG)#(eC)#(eU)#(eC), and
(eC)#(eC)#(eT)#(eC)#(eG)#(eC)#(eC)#(eC)#(eA)#(eG)#(eA)#(eA)#(eC)#(eA)#(eG)#(eT) #(eG)#(eG)#(eA)#(eG) or (eC)#(eC)#(eU)#(eC)#(eG)#(eC)#(eC)#(eC)#(eA)#(eG)#(eA)#(eA)#(eC)#(eA)#(eG) #(eU)#(eG)#(eG)#(eA)#(eG),
wherein e is a 2′-O-methoxyethyl ribose sugar, and wherein # is a phosphorothioate internucleotide linkage.

In some embodiments, an ASO comprises:
a nucleotide sequence having at least 70% sequence identity to SEQ ID NO:10, for example, having at least: 75%, 80%, 85%, 88%, 90%, 92%, 95%, 96%, 97%, 98%, or 99% sequence identity to SEQ ID NO:10; and
a nucleotide sequence having at least 70% sequence identity to SEQ ID NO:11, for example, having at least: 75%, 80%, 85%, 88%, 90%, 92%, 95%, 96%, 97%, 98%, or 99% sequence identity to SEQ ID NO:11.

In some embodiments, an ASO comprises:
a nucleotide sequence having at least 85% sequence identity to SEQ ID NO: 10; and
a nucleotide sequence having at least 85% sequence identity to SEQ ID NO: 11.

In some embodiments, an ASO comprises:
a nucleotide sequence having 100% sequence identity to SEQ ID NO:10; and
a nucleotide sequence having 100% sequence identity to SEQ ID NO:11.

In some embodiments, an ASO comprises:
(eC)#(eG)#(eC)#(eC)#(eC)#(eA)#(eG)#(eA)#(eA)#(eC)#(eA)#(eG)#(eT)#(eG)#(eG)#(e A)#(eG)#(eC)#(eT)#(eC), and
(eC)#(eC)#(eT)#(eC)#(eG)#(eC)#(eC)#(eC)#(eA)#(eG)#(eA)#(eA)#(eC)#(eA)#(eG)#(eT) #(eG)#(eG)#(eA)#(eG),
wherein e is a 2′-O-methoxyethyl ribose sugar, and wherein # is a phosphorothioate internucleotide linkage.

In some embodiments, an ASO comprises:
a nucleotide sequence having at least 70% sequence identity to SEQ ID NO:60, for example, having at least: 75%, 80%, 85%, 88%, 90%, 92%, 95%, 96%, 97%, 98%, or 99% sequence identity to SEQ ID NO:10 or SEQ ID NO:60; and
a nucleotide sequence having at least 70% sequence identity to SEQ ID NO:61, for example, having at least: 75%, 80%, 85%, 88%, 90%, 92%, 95%, 96%, 97%, 98%, or 99% sequence identity to SEQ ID NO:61.

In some embodiments, an ASO comprises:
a nucleotide sequence having at least 85% sequence identity to SEQ ID NO:60; and
a nucleotide sequence having at least 85% sequence identity to SEQ ID NO:61.

In some embodiments, an ASO comprises:
a nucleotide sequence having 100% sequence identity to SEQ ID NO:60; and
a nucleotide sequence having 100% sequence identity to SEQ ID NO:61.

In some embodiments, an ASO comprises:
(eC)#(eG)#(eC)#(eC)#(eC)#(eA)#(eG)#(eA)#(eA)#(eC)#(eA)#(eG)#(eU)#(eG)#(eG)#(e A)#(eG)#(eC)#(eU)#(eC), and
(eC)#(eC)#(eU)#(eC)#(eG)#(eC)#(eC)#(eC)#(eA)#(eG)#(eA)#(eA)#(eC)#(eA)#(eG)#(e U)#(eG)#(eG)#(eA)#(eG),
wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:70 or SEQ ID NO:73, for example, having at least: 75%, 80%, 85%, 88%, 90%, 92%, 95%, 96%, 97%, 98%, or 99% sequence identity to SEQ ID NO:70 or SEQ ID NO:73. In some embodiments, an ASO comprises a nucleotide sequence having at least 85% sequence identity to SEQ ID NO:70 or SEQ ID NO:73. In some embodiments, an ASO comprises a nucleotide sequence having 100% sequence identity to SEQ ID NO:70 or SEQ ID NO:73.

In some embodiments, an ASO comprises
(eC)#(eC)#(eT)#(eC)#(eG)#(eC)#(eC)#(eC)#(eA)#(eG)#(eA)#(eA)#(eC)#(eA)#(eG)#(eT) #(eG)#(eG)#(eA)#(eG)#(eC)#(eT)#(eC), and/or
(eC)#(eC)#(eU)#(eC)#(eG)#(eC)#(eC)#(eC)#(eA)#(eG)#(eA)#(eA)#(eC)#(eA)#(eG)#(e U)#(eG)#(eG)#(eA)#(eG)#(eC)#(eU)#(eC),
wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:70, for example, having at least: 75%, 80%, 85%, 88%, 90%, 92%, 95%, 96%, 97%, 98%, or 99% sequence identity to SEQ ID NO:70. In some embodiments, an ASO comprises a nucleotide sequence having at least 85% sequence identity to SEQ ID NO:70. In some embodiments, an ASO comprises a nucleotide sequence having 100% sequence identity to SEQ ID NO:70.

In some embodiments, an ASO comprises
(eC)#(eC)#(eT)#(eC)#(eG)#(eC)#(eC)#(eC)#(eA)#(eG)#(eA)#(eA)#(eC)#(eA)#(eG)#(eT)#(eG) #(eG)#(eA)#(eG)#(eC)#(eT)#(eC), wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:73, for example, having at least: 75%, 80%, 85%, 88%, 90%, 92%, 95%, 96%, 97%, 98%, or 99% sequence identity to SEQ ID NO:73. In some embodiments, an ASO comprises a nucleotide sequence having at least 85% sequence identity to SEQ ID NO:73. In some embodiments, an ASO comprises a nucleotide sequence having 100% sequence identity to SEQ ID NO:73.

In some embodiments, an ASO comprises
(eC)#(eC)#(eU)#(eC)#(eG)#(eC)#(eC)#(eC)#(eA)#(eG)#(eA)#(eA)#(eC)#(eA)#(eG)#(eU)#(eG) #(eG)#(eA)#(eG)#(eC)#(eU)#(eC), wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:71 or SEQ ID NO:74, for example, having at least: 75%, 80%, 85%, 88%, 90%, 92%, 95%, 96%, 97%, 98%, or 99% sequence identity to SEQ ID NO:71 or SEQ ID NO:74. In some embodiments, an ASO comprises a nucleotide sequence having at least 85% sequence identity to SEQ ID NO:71 or SEQ ID NO:74. In some embodiments, an ASO comprises a nucleotide sequence having 100% sequence identity to SEQ ID NO:71 or SEQ ID NO:74.

In some embodiments, an ASO comprises
(eG)#(eC)#(eC)#(eC)#(eA)#(eG)#(eA)#(eA)#(eC)#(eA)#(eG)#(eT)#(eG)#(eG)#(eA)#(e G)#(eC)#(eT)#(eC), and/or
(eG)#(eC)#(eC)#(eC)#(eA)#(eG)#(eA)#(eA)#(eC)#(eA)#(eG)#(eU)#(eG)#(eG)#(eA)#(e G)#(eC)#(eU)#(eC),
wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:71, for example, having at least: 75%, 80%, 85%, 88%, 90%, 92%, 95%, 96%, 97%, 98%, or 99% sequence identity to SEQ ID NO:71. In some embodiments, an ASO comprises a nucleotide sequence having at least 85% sequence identity to SEQ ID NO:71. In some embodiments, an ASO comprises a nucleotide sequence having 100% sequence identity to SEQ ID NO:71.

In some embodiments, an ASO comprises
(eG)#(eC)#(eC)#(eC)#(eA)#(eG)#(eA)#(eA)#(eC)#(eA)#(eG)#(eT)#(eG)#(eG)#(eA)#(eG)#(eC) #(eT)#(eC), wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:74, for example, having at least: 75%, 80%, 85%, 88%, 90%, 92%, 95%, 96%, 97%, 98%, or 99% sequence identity to SEQ ID NO:74. In some embodiments, an ASO comprises a nucleotide sequence having at least 85% sequence identity to SEQ ID NO:74. In some embodiments, an ASO comprises a nucleotide sequence having 100% sequence identity to SEQ ID NO:74.

In some embodiments, an ASO comprises
(eG)#(eC)#(eC)#(eC)#(eA)#(eG)#(eA)#(eA)#(eC)#(eA)#(eG)#(eU)#(eG)#(eG)#(eA)#(eG)#(eC) #(eU)#(eC), wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:72 or SEQ ID NO:75, for example, having at least: 75%, 80%, 85%, 88%, 90%, 92%, 95%, 96%, 97%, 98%, or 99% sequence identity to SEQ ID NO:72 or SEQ ID NO:75. In some embodiments, an ASO comprises a nucleotide sequence having at least 85% sequence identity to SEQ ID NO:72 or SEQ ID NO:75. In some embodiments, an ASO comprises a nucleotide sequence having 100% sequence identity to SEQ ID NO:72 or SEQ ID NO:75.

In some embodiments, an ASO comprises
(eC)#(eC)#(eT)#(eC)#(eG)#(eC)#(eC)#(eC)#(eA)#(eG)#(eA)#(eA)#(eC)#(eA)#(eG)#(eT) #(eG)#(eG)#(eA),
(eC)#(eC)#(eU)#(eC)#(eG)#(eC)#(eC)#(eC)#(eA)#(eG)#(eA)#(eA)#(eC)#(eA)#(eG)#(e U)#(eG)#(eG)#(eA),
(eC)#(eC)#(eT)#(eC)(eG)(eC)(eC)#(eC)#(eA)#(eG)#(eA)#(eA)#(eC)(eA)(eG)(eT)#(eG) #(eG)#(eA), and/or
(eC)#(eC)#(eU)#(eC)(eG)(eC)(eC)#(eC)#(eA)#(eG)#(eA)#(eA)#(eC)(eA)(eG)(eU)#(eG) #(eG)#(eA),
wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:72, for example, having at least: 75%, 80%, 85%, 88%, 90%, 92%, 95%, 96%, 97%, 98%, or 99% sequence identity to SEQ ID NO:72. In some embodiments, an ASO comprises a nucleotide sequence having at least 85% sequence identity to SEQ ID NO:72. In some embodiments, an ASO comprises a nucleotide sequence having 100% sequence identity to SEQ ID NO:72.

In some embodiments, an ASO comprises:
(eC)#(eC)#(eT)#(eC)#(eG)#(eC)#(eC)#(eC)#(eA)#(eG)#(eA)#(eA)#(eC)#(eA)#(eG)#(eT) #(eG)#(eG)#(eA), and/or
(eC)#(eC)#(eT)#(eC)(eG)(eC)(eC)#(eC)#(eA)#(eG)#(eA)#(eA)#(eC)(eA)(eG)(eT)#(eG) #(eG)#(eA), wherein e is a 2′-O-methoxyethyl ribose sugar, and wherein # is a phosphorothioate internucleotide linkage.

In some embodiments, an ASO comprises
(eC)#(eC)#(eT)#(eC)#(eG)#(eC)#(eC)#(eC)#(eA)#(eG)#(eA)#(eA)#(eC)#(eA)#(eG)#(eT)#(eG) #(eG)#(eA), wherein e is a 2′-O-methoxyethyl ribose sugar, and wherein # is a phosphorothioate internucleotide linkage.

In some embodiments, an ASO comprises
(eC)#(eC)#(eT)#(eC)(eG)(eC)(eC)#(eC)#(eA)#(eG)#(eA)#(eA)#(eC)(eA)(eG)(eT)#(eG)#(eG)#(eA), wherein e is a 2′-O-methoxyethyl ribose sugar, and wherein # is a phosphorothioate internucleotide linkage.

In some embodiments, an ASO comprises a nucleotide sequence having at least 70% sequence identity to SEQ ID NO:75, for example, having at least: 75%, 80%, 85%, 88%, 90%, 92%, 95%, 96%, 97%, 98%, or 99% sequence identity to SEQ ID NO:75. In some embodiments, an ASO comprises a nucleotide sequence having at least 85% sequence identity to SEQ ID NO:75. In some embodiments, an ASO comprises a nucleotide sequence having 100% sequence identity to SEQ ID NO:75.

In some embodiments, an ASO comprises:
(eC)#(eC)#(eU)#(eC)#(eG)#(eC)#(eC)#(eC)#(eA)#(eG)#(eA)#(eA)#(eC)#(eA)#(eG)#(e U)#(eG)#(eG)#(eA), and/or
(eC)#(eC)#(eU)#(eC)(eG)(eC)(eC)#(eC)#(eA)#(eG)#(eA)#(eA)#(eC)(eA)(eG)(eU)#(eG) #(eG)#(eA),
wherein e is a 2′-O-methoxyethyl ribose sugar, and wherein # is a phosphorothioate internucleotide linkage.

In some embodiments, an ASO comprises
(eC)#(eC)#(eU)#(eC)#(eG)#(eC)#(eC)#(eC)#(eA)#(eG)#(eA)#(eA)#(eC)#(eA)#(eG)#(eU)#(eG) #(eG)#(eA), wherein e is a 2′-O-methoxyethyl ribose sugar, and wherein # is a phosphorothioate internucleotide linkage.

In some embodiments, an ASO comprises
(eC)#(eC)#(eU)#(eC)(eG)(eC)(eC)#(eC)#(eA)#(eG)#(eA)#(eA)#(eC)(eA)(eG)(eU)#(eG)#(eG)#(eA), wherein e is a 2′-O-methoxyethyl ribose sugar, and wherein # is a phosphorothioate internucleotide linkage.

In another aspect, the present disclosure provides a pharmaceutical composition, comprising at least one ASO disclosed herein and a pharmaceutically acceptable excipient, diluent, and/or carrier. In some embodiments, a pharmaceutical composition comprises at least two ASOs disclosed herein.

In another aspect, the present disclosure provides a method of treating a disease in a subject in need thereof, comprising administering to the subject a therapeutically effective amount of any one of the pharmaceutical compositions disclosed herein. In some embodiments, a disease is a fragile X-associated disorder. In some embodiments, a fragile X-associated disorder is fragile X syndrome (FXS), fragile X-associated primary ovarian insufficiency (FXPOI), or fragile X-associated tremor/ataxia syndrome (FXTAS). In some embodiments, a fragile X-associated disorder is FXS.

In some embodiments, a therapeutically effective amount of a pharmaceutical composition decreases an aberrant FMR1 transcript, decreases a protein encoded by an aberrant FMR1 transcript, or both. In some embodiments, aberrant FMR1 transcript comprises a FMR1-217 transcript.

In some embodiments, a therapeutically effective amount of a pharmaceutical composition decreases a FMR1-217 transcript. In some embodiments, a therapeutically effective amount of a pharmaceutical composition decreases a FMR1-217 transcript by at least about 5%, e.g., by at least about: 10%, 15%, 20%, 25%, 30%, 35%, 40%, 45%, 50%, 55%, 60%, 65%, 70%, 75%, 80%, 85%, 90%, 95% or 98%, relative to a reference. In some embodiments, a therapeutically effective amount of a pharmaceutical composition decreases a FMR1-217 transcript by at least 20%, relative to a reference.

In some embodiments, a therapeutically effective amount of a pharmaceutical composition increases expression of fragile X messenger ribonucleoprotein (FMRP). In some embodiments, a therapeutically effective amount of a pharmaceutical composition increases expression of FMRP by at least about 5%, e.g., by at least about: 10%, 15%, 20%, 25%, 30%, 35%, 40%, 45%, 50%, 55%, 60%, 65%, 70%, 75%, 80%, 85%, 90%, 95% or 98%, relative to a reference. In some embodiments, a therapeutically effective amount of a pharmaceutical composition increases expression of FMRP by at least 25%, relative to a reference.

In another aspect, the present disclosure provides a method of reducing a FMR1-217 transcript in a cell, comprising contacting the cell with an effective amount of the at least one ASO disclosed herein or any one of the pharmaceutical compositions disclosed herein.

In some embodiments, a method of reducing a FMR1-217 transcript in a cell, comprises contacting the cell with an ASO or a pharmaceutical composition in the presence of a transfection agent. In some embodiments, a transfection agent is a LIPOFECTAMINE® reagent, for example, LIPOFECTAMINE® 2000, LIPOFECTAMINE® 3000, LIPOFECTAMINE® LTX, LIPOFECTAMINE® RNAiMAX, or LIPOFECTAMINE® CRISPRMAX. In some embodiments, a transfection reagent comprises an INVIVOFECTAMINE® reagent for in vivo transfection. In some embodiments, a transfection reagent comprises LIPOFECTAMINE® or LIPOFECTAMINE® 2000.

In some embodiments, an effective amount of an ASO or a pharmaceutical composition decreases a FMR1-217 transcript. In some embodiments, an effective amount of an ASO or a pharmaceutical composition decreases a FMR1-217 transcript by at least about 5%, e.g., by at least about: 10%, 15%, 20%, 25%, 30%, 35%, 40%, 45%, 50%, 55%, 60%, 65%, 70%, 75%, 80%, 85%, 90%, 95% or 98%, relative to a reference. In some embodiments, an effective amount of an ASO or a pharmaceutical composition decreases a FMR1-217 transcript by at least 25%, relative to a reference.

In some embodiments, an effective amount of an ASO or a pharmaceutical composition increases expression of fragile X messenger ribonucleoprotein (FMRP). In some embodiments, an effective amount of an ASO or a pharmaceutical composition increases expression of FMRP by at least about 5%, e.g., by at least about: 10%, 15%, 20%, 25%, 30%, 35%, 40%, 45%, 50%, 55%, 60%, 65%, 70%, 75%, 80%, 85%, 90%, 95% or 98%, relative to a reference. In some embodiments, an effective amount of an ASO or a pharmaceutical composition increases expression of FMRP by at least 25%, relative to a reference.

In some embodiments, a cell is derived from or in a subject having a fragile X-associated disorder. In some embodiments, a fragile X-associated disorder is fragile X syndrome (FXS), fragile X-associated primary ovarian insufficiency (FXPOI), or fragile X-associated tremor/ataxia syndrome (FXTAS). In some embodiments, a fragile X-associated disorder is FXS.

### EXEMPLIFICATION

Most FXS studies are focused on Fmr1 knockout (KO) mouse models. Shah et al. shows that Fmr1 KO mice have dysregulated pre-mRNA splicing in the brain (Shah et al., FMRP Control of Ribosome Translocation Promotes Chromatin Modifications and Alternative Splicing of Neuronal Genes Linked to Autism, Cell Rep. 30(13):4459-72 (2020)).

New data show that missplicing in the FMRP KO mouse occurs in all brain regions and peripheral tissues tested. Therefore, because FMRP is likely present in all cells, missplicing probably also occurs in all cells.

### Example 1. Methods

### RNA Extraction and Sequencing

RNA was extracted from patient leukocytes using the LeukoLOCK™ total RNA isolation system (AM1923, Thermo Fisher Scientific, Waltham, MA). Ten mL fresh blood was collected from FXS male patients (N=10) and age-matched typically developing males (N=7) (controls) in an anti-coagulant containing tube, and RNA was extracted using a LeukoLOCK™ fractionation & stabilization kit (AM1933, Thermo Fisher Scientific, Waltham, MA), per the manufacturer's instructions. Briefly, the blood sample was passed through a LeukoLOCK™ filter and 3 mL phosphate buffered saline (PBS) was used to rinse the filter followed by 3 mL of RNALATER® RNA Stabilization Solution (Thermo Fisher Scientific, Waltham, MA). The residual RNALATER® was expelled from the LeukoLOCK™ filter and the filters were capped and stored at −80° C.

To extract RNA, the filters were thawed at room temperature for 5 minutes and then the remaining RNALATER® was removed. The filter was flushed with 4 mL of TRI Reagent, and the lysate was collected in a 15-mL tube. 800 μl 1-Bromo-3-chloropropane (BCP) was added to each tube and vortexed vigorously for 30 seconds. The tube was then incubated at room temperature for 5 minutes. After centrifugation for 10 minutes at 4° C. at ˜2,000×g, the aqueous phase was recovered. To recover long RNA fractions, 0.5 volumes of 100% ethanol were added and mixed well. The RNA was then recovered using the RNA clean and concentrator kit. DNase treatment was performed using Turbo™ DNase (Thermo Fisher Scientific, Waltham, MA), and the RNA obtained was resuspended in RNAse free water and stored at −80° C. 1 μg of the RNA was used for cDNA synthesis using the QuantiTect® reverse transcription kit (Qiagen, Hilden, Germany) to assess for depletion of the Globin mRNA using qPCR, to confirm exclusion of red blood cells from the prep. 3 μg of RNA sample was sent to Novogene (Beijing, China) for a directional mRNA library preparation using polyA enrichment. The libraries were sequenced on the NovaSeq platform to generate paired end, 150 bp reads.

### RNA-Seq Data Analysis

Fastq files were uploaded to the DolphinNext platform (Yukselen et al., Dolphin Next: a distributed data processing platform for high throughput genomics, BMC Genomics 21(1):310 (2020)) at the University of Massachusetts Chan Medical School (UMMS) Bioinformatics Core for mapping and quantification. The reads were subjected to fastqc pipeline, and the quality of reads was assessed. 9-nt molecular labels were trimmed from both 5′ends of the pair-end reads and quality-filtered with Trimmomatic (0.32). Reads mapped to human rRNA by Bowtie2 (2.1.0) were filtered out. Cleaned reads were next mapped to the Refseq (V38) human transcriptome and quantified by RSEM (1.2.11). Estimated counts on each gene were used for the differential gene expression analysis by DESeq2 (1.16.1). After the normalization by median of ratios method, only the genes with minimal 5 counts average across all samples were kept for the Differential Gene expression analysis. The FDR (padj) cut-off<5% was used. The TDF files generated were uploaded on the Integrative Genomics Viewer for visualization.

The ratio between reads including or excluding exons, also known as “Percent Spliced In” (PSI), indicates how efficiently sequences of interest are spliced into transcripts. The False Discovery Rate (FDR) is a method of conceptualizing the rate of type I errors in null hypothesis testing when conducting multiple comparisons.

### Alternative Splicing Analysis

RNA-seq data generated from leukocytes from FXS male patients (N=10) and age-matched typically developing males (N=7) was used to analyze alternative splicing (AS) using the rMATS package v3.2.5 (Shen et al., rMATS: Robust and flexible detection of differential alternative splicing from replicate RNA-Seq data, Proc. Natl. Acad. Sci. 111(51):E5593-5601 (2014)) with default parameters. The Percent Spliced In (PSI) levels or the exon inclusion levels were calculated by rMATS using a hierarchical framework. To calculate the difference in PSI between genotypes, a likelihood-ratio test was used. AS events with an FDR<5% and |deltaPSI| ≥5% as identified using rMATS were used for further analysis.

### Primer Sets for Detecting FMR1 Isoforms

Iso1_1Forward (Iso1_1 F):
(SEQ ID NO: 12)
5′ AGAAGATGGAGGAGCTGGTG 3′
Iso12_1Reverse (Iso12_1 R):
(SEQ ID NO: 13)
5′ CAGTGGAGCTCTCCGAAGTC 3′
Iso12_2Forward:
(SEQ ID NO: 14)
5′ CCAGCAGTGCATTGAAGAAG 3′
Iso12_2Reverse:
(SEQ ID NO: 15)
5′ CTGAAGCATGTGCATTCCTG 3′
Iso1_1 Forward (Iso1_1 F):
(SEQ ID NO: 12)
5′ AGAAGATGGAGGAGCTGGTG 3′
Iso1_1 Reverse (Iso1_1 R):
(SEQ ID NO: 16)
5′ TTCATGAACATCCTTTACAAATGC 3′
Exon1 Forward (Exon1 F):
(SEQ ID NO: 17)
5′ TAGCAGGGCTGAAGAGAA 3′
Exon1 Reverse (Exon1 R):
(SEQ ID NO: 18)
5′ CTTGTAGAAAGCGCCATTG 3′

### Detection of FMR1 Isoforms

A white blood cell line derived from an FXS patient who expressed iso12 was transfected with antisense oligonucleotides (ASOs) pairs 705/705, 709/710, and 713/714. RNA was extracted 48 hours later and subjected to RT-qPCR to detect iso1 (primers Iso1_1 Forward/Iso1_1 Reverse) or total FMR1 isoforms (iso1+iso12) (primers Exon1 Forward and Exon1 Reverse) and iso12 (primers Iso1_1 Forward/Iso12_1 Reverse). Each assay was performed in triplicate and normalized against non-transfected cells.

### Cell Culture

### Cell Lines and Treatments

Lymphoblastoid cell lines (LCL) were obtained from Coriell Institute from two FXS individuals (GM07365 (FXS1), GM06897(FXS2)) and two typically developing control males (GM07174 (WT3), GM06890 (WT4)). Cells were cultured in RPMI 1640 medium (Sigma-Aldrich, St. Louis, MO), supplemented with 15% fetal bovine serum (FBS) and 2.5% L-glutamine at 37° C. with 5% CO₂ in T25 flasks.

Fibroblast cells derived from patient skin samples were cultured in DMEM (15-017-CV) medium supplemented with 10% FBS and 1×antibiotic-antimitotic, 1×L-glutamine in T25 culture flasks at 37° C. with 5% CO₂.

### ASO Treatment

Antisense oligonucleotides (ASOs) were dissolved in ultrapure distilled water to a final concentration of 10 μM. Before use, the ASOs were heated to 55° C. for 15 minutes and cooled at room temperature. ASOs were added individually or in combinations to LCL cell lines at a final concentration of 80 nM using Lipofectamine® RNAIMAX® Transfection Reagent (Thermo Fisher Scientific, Waltham, MA, #13778030) and incubated at 37° C. with 5% CO₂ for 16 hrs in reduced serum medium. RPMI 1640 medium (Sigma-Aldrich, St. Louis, MO), supplemented with 15% FBS was added for a total of 48 hours. The cells were collected after 48 hours of ASO treatment for RNA and protein extraction.

### 5-AzaC Treatment

For each cell culture, 30×10⁵ cells/mL were added in a final volume of 20 mL medium (RPMI 1640 medium (Sigma-Aldrich), supplemented with 15% FBS and 2.5% L-glutamine at 37° C. with 5% CO₂) per T25 flask. 5-Aza-2′-deoxycytidine (5-AzaC) (Sigma-Aldrich, A3656) was added to the cell cultures (final concentration 1 μM) for 7 consecutive days. A 2 mM stock of 5-AzaC was made in DMSO. For each cell line, two independent treatments were performed (n=2). For the no treatment controls for each cell line, DMSO was added to the flasks. For samples with both 5-AzaC and ASO treatment, 80 nM ASOs or vehicle were added on Day 1 and either 5-AzaC or DMSO was added each day from Day 2 up to Day 9 at a final concentration of 1 μM. On Day 9 the cells were collected in 1×Phosphate buffered saline to proceed with RNA extraction or Western blotting.

### Western Blotting

Cells were homogenized at 4° C. in RIPA buffer with incubation on ice for 10 minutes and dissociation by pipetting. The extract was centrifuged at 13,200 rpm for 10 minutes at 4° C. and the supernatant collected. Protein concentration was determined by BCA reagent. Proteins (10 μg) were diluted in SDS-bromophenol blue reducing buffer with 40 mM DTT and analyzed using western blotting on a 10% SDS-PAGE gel with the following antibodies: FMRP (Abcam, 1:2000) and GAPDH (Cell signaling, 1:2000) diluted in 1×TBST with 5% non-fat milk. Membranes were washed three times for 10 minutes with 1×TBST and incubated with anti-rabbit or anti-mouse secondary antibodies (Jackson, 1:10000) at room temperature for 1 hour. Membranes were washed three times for 10 minutes with 1×TBST, developed with ECL-Plus (Piece), and scanned with GE Amersham Imager.

### Example 2. FMR1 Isoform 12 Detected in a Subpopulation of FXS Patients

FXS is caused by a CGG triplet repeat expansion in a single gene, FMR1, which resides on the X chromosome. When the CGG triplet expands to 200 or more, the FMR1 gene is methylated and thereby transcriptionally inactivated. The loss of the FMR1 gene product, the protein FMRP, is the cause of the disorder.

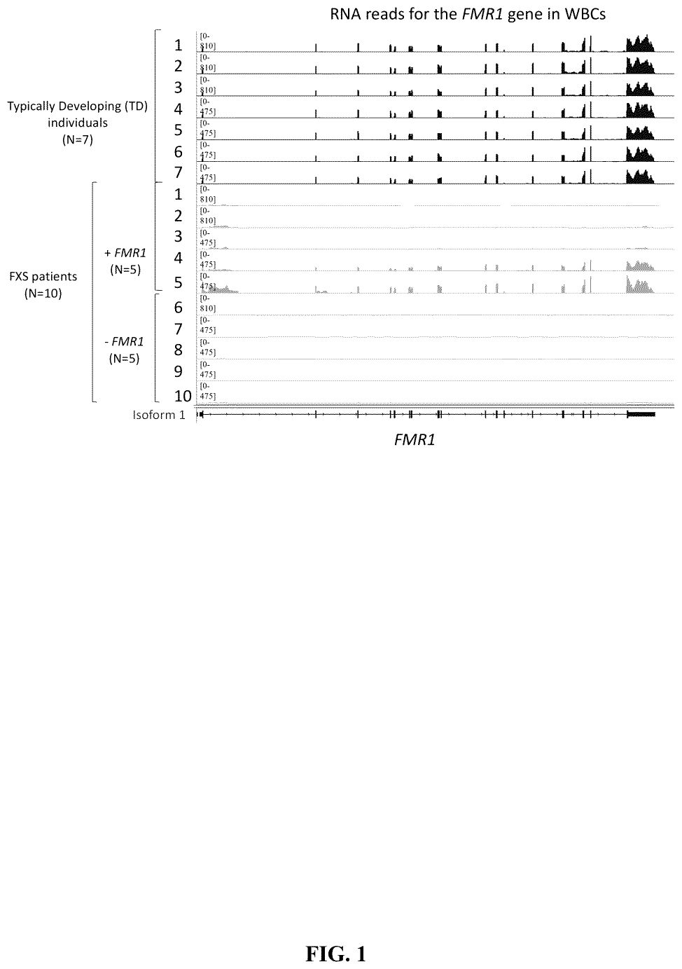

Bioinformatic analysis showed that one-half of the FXS patients expressed detectable levels of FMR1 RNA, which was unexpected given that all patients had greater than 200 CGG repeats and had been clinically diagnosed with fragile X syndrome. This detection of FMR1 RNA in one-half of the FXS patients indicated that these individuals had incomplete DNA methylation of FMR1, because it is DNA methylation that silences the gene. FIG. 1 shows that there was robust expression of FMR1 in all 7 typically developing (TD) individuals. There was also FMR1 expression in FXS patients 1-5 (+FMR1), but no FMR1 expression was detected in FXS patients 6-10 (−FMR1). Therefore, 50% of FXS individuals express FMR1 RNA, likely due to incomplete methylation.

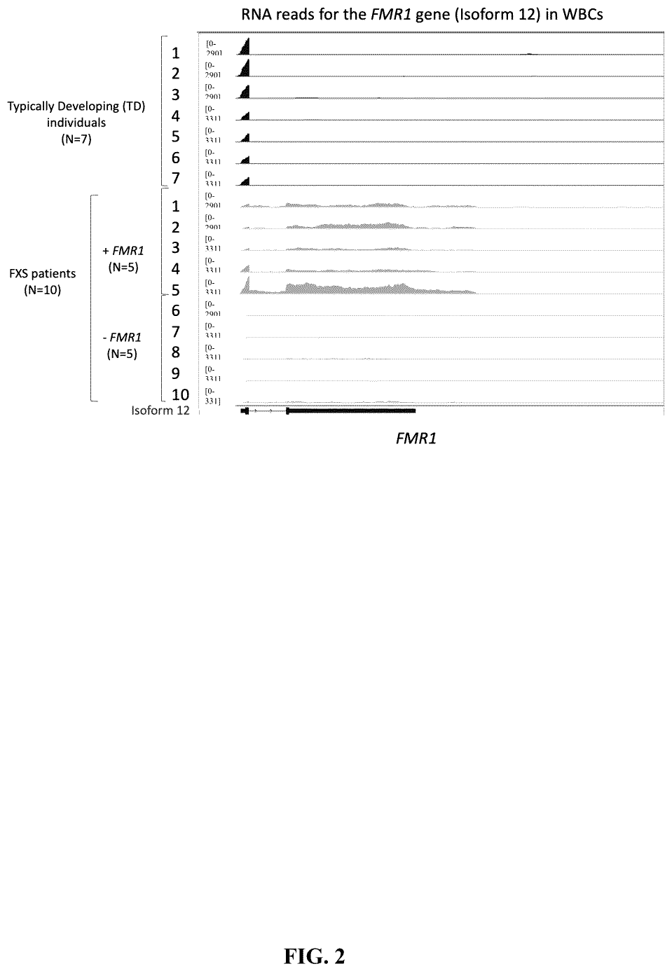

In the fragile X syndrome patients who did express FMR1 RNA, further bioinformatic analysis showed that the FMR1 RNA was misspliced. That is, instead of, or in addition to proper FMR1 splicing, there was a little-known isoform derived from missplicing. Normally, FMR1 exon 1 (chrX: 147,911,919-147,912,230) is spliced to FMR1 exon 2 (chrX: 147,921,933-147,921,985), which produces “isoform 1” or “Iso1.” However, within intron 1, there is a pseudo exon (chrX: 147,912,728-147,914,451), and splicing between FMR1 exon 1 and this pseudo exon produces “isoform 12” or “Iso12.” FIG. 2 shows an expanded view of FMR1 exon 1 and intron 1. Note that although none of the typically developing individuals expresses isoform 12, the five FXS patients who expressed FMR1 RNA (+FMR1) all express FMR1 isoform 12.

Isoform 12 is derived from missplicing, detected only when there was a CGG repeat expansion and when there was incomplete methylation. Isoform 12 does not produce full-length or functional FMRP. Instead, isoform 12 generates a 30-amino acid protein, which probably has no biological function.

These findings suggest that FMR1 RNA not only can be used for diagnosing an individual as having FXS, or having a propensity to develop FXS, but also can be used for stratifying FXS individuals. The identification FMR1 RNA isoform 12 enables stratification of FXS individuals into two subpopulations, those who express isoform 12 and those who do not.

These findings further suggest that FMR1 RNA, such as isoform 12, may provide novel therapeutic targets for FXS. For example, a reduction of aberrant splicing to isoform 12, alone or commensurate with an increase of proper splicing to isoform 1 (i.e., normal FMR1 RNA with exon 1 spliced to exon 2), may increase FMRP levels and thereby mitigate FXS in patients who express FMR1 RNA. In patients who does not express FMR1 RNA, it may be feasible to generate isoform 12 with a therapeutically effective amount of a DNA-demethylating compound or DNA demethylase, which could ideally include a targeted approach to partially demethylate the FMR1 gene without inducing general, widespread DNA demethylation.

### Example 3. Reducing Isoform 12 Production and Increasing Isoform 1 Production

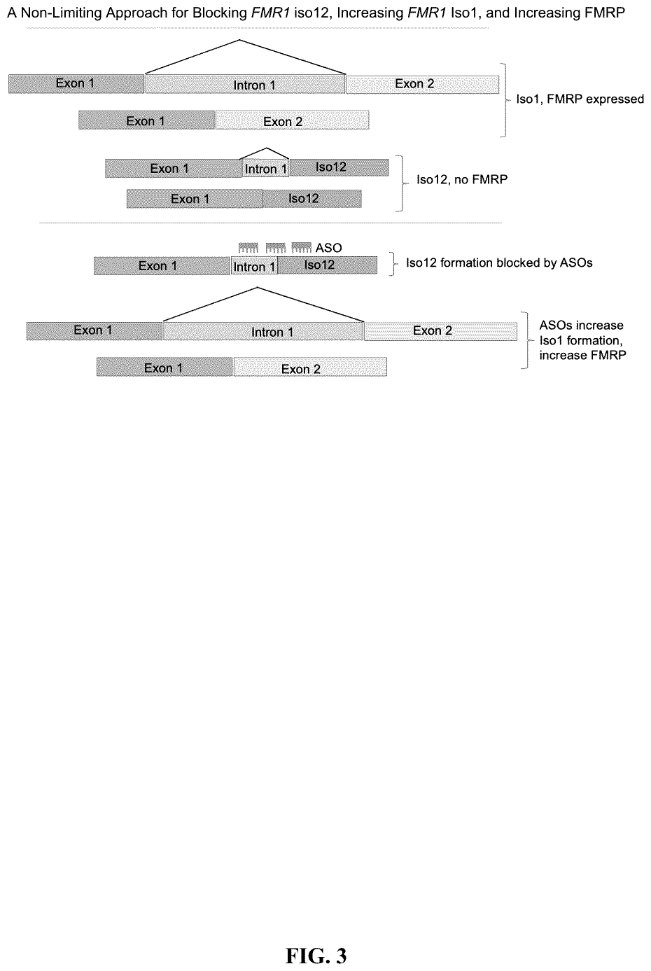

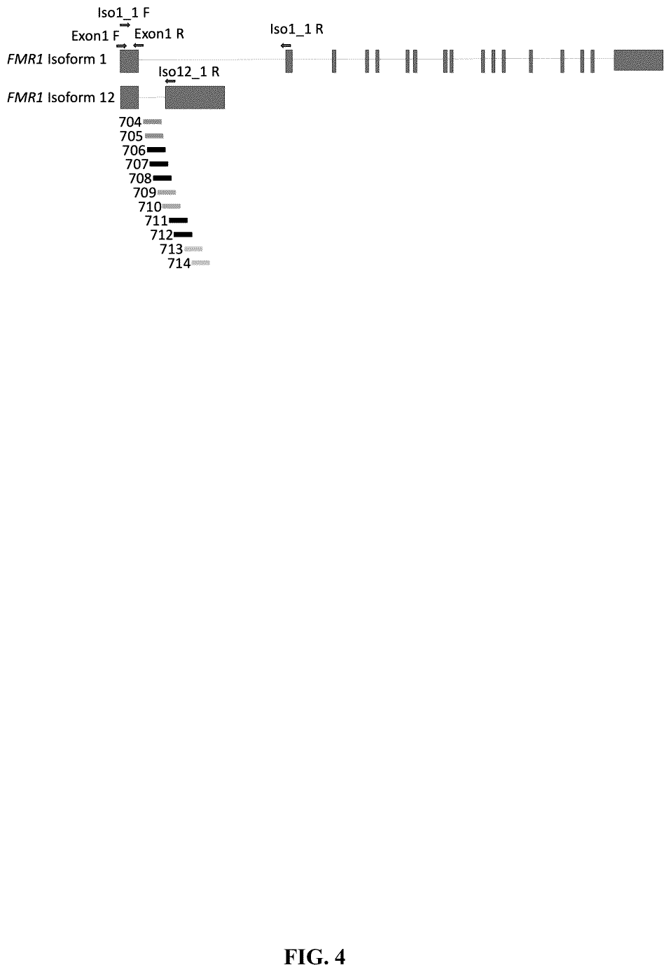

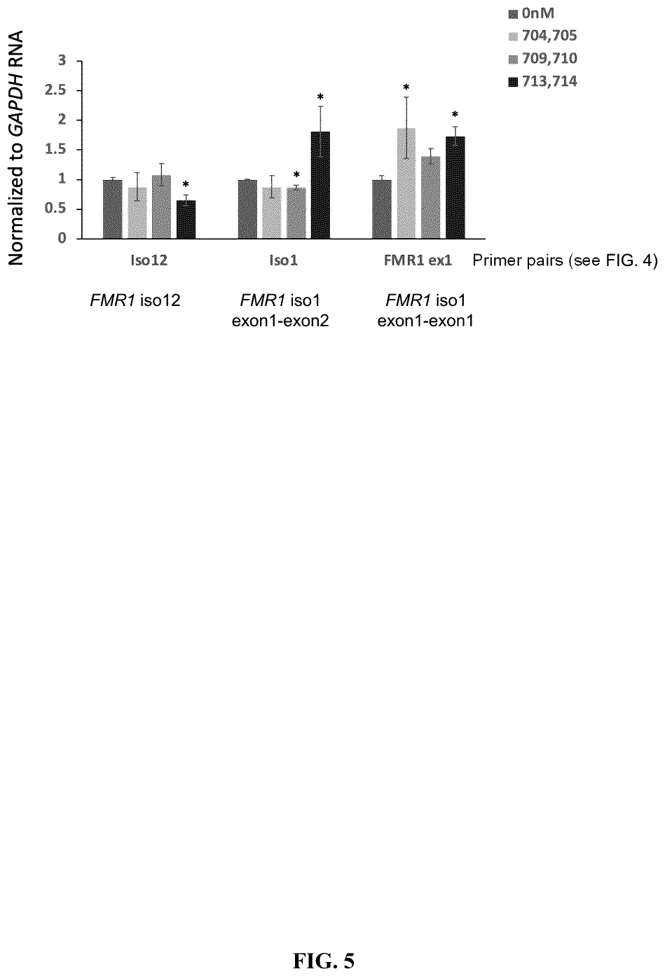

FIG. 3 shows a non-limiting example approach for blocking isoform 12 production, increasing isoform 1 production, and increasing FMRP levels using antisense oligonucleotides (ASOs). ASOs were designed to be complementary to regions within intron 1 and upstream of isoform 12, the junction spanning intron 1 and isoform 12, or within isoform 12 (Table 1). FIG. 4 shows a schematic illustration of FMR1 iso1, iso12, and relative positions of ASOs complementary to intron 1 (704, 705, and 706), the junction of intron 1 and iso12 (707, 708, 709, and 710), and within iso12 (711, 712, 713, and 714).

ASOs 704-714 were chemically modified to increase the nuclease resistance of the ASOs (e.g., reduce RNase H cleavage), increase cellular uptake, and enhance base-pairing capabilities (reduce off-target effects). The ribose or deoxyribose groups comprised 2′-O-(2-methoxyethyl) (MOE), and the phosphate groups comprised a phosphorothioate.

ASOs of the disclosure may be used singly or in combination. A WBC line derived from a FXS patient who expressed iso12 was transfected with ASOs 704/705, 709/710 or 713/714. RNA was extracted 48 hours later and subjected to RT-qPCR to detect iso1 (primers Iso1_1 Forward and Iso1_1 Reverse) and iso12 (primers Iso1_1 Forward and Iso12_1 Reverse). Each assay was performed in triplicate. FIG. 5 illustrates that ASOs 713 and 714, both of which are complementary to internal regions of iso12, reduced the iso12 level by ˜30% and increased the iso1 level by ˜75%. These data indicate that ASOs can be used to reduce isoform 12 expression. More importantly, these data indicate that ASOs can be used to elevate FMR1 isoform 1 expression, which may in turn increase FMRP levels and mitigate FXS.

These data suggest that ASOs may be a potent and specific therapeutic to treat a subpopulation of FXS individuals that express isoform 12. The findings provide further support that agents, such as ASOs, directed against FMR1 isoform 12 may provide novel therapeutic treatment to FXS by reducing improper splicing to isoform 12, increasing proper splicing of isoform 1 and increasing FMRP levels. This approach is entirely novel in the fragile X field. It is predicted to be a significant improvement over the prior art because all other treatments for FXS elicit only modest improvements at best. Additionally, all other therapies treat FXS patients as one large cohort, whereas these studies have identified a particular subpopulation—those who express iso12—and may be particularly amenable to therapeutics, such as ASOs that target iso12.

### Example 4. Partial Demethylation of FMR1 DNA

Experiments illustrated in Example 3 have been and will be performed in cells with different methylation status.

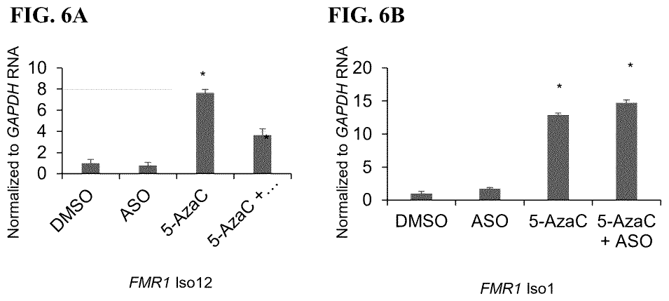

FIG. 6A shows RT-qPCR data from a fully methylated FXS cell line (FXS1, GM07365). The FMR1 locus in this cell line is silenced and thus the FMR1 RNA (iso1 and iso12) and FMRP protein levels are very low compared to the FXS2 cell line with an unmethylated FMR1 gene. Treatment with the demethylating agent 5-AzaC resulted in demethylation of the FMR1 gene to allow expression of the FMR1 RNA isoforms. The data demonstrate an increase in FMR1 iso12 upon 5-AzaC treatment (p<0.05) and a partial rescue of the FMR1 iso12 increase when the 5-AzaC treatment was combined with the ASO treatment (80 nM of both antisense oligonucleotides 713 and 714) (p<0.05). FIG. 6B demonstrates an increase in FMR1 iso1 upon 5-AzaC treatment (p<0.05) and a further increase when the ASO treatment (80 nM of both antisense oligonucleotides 713 and 714) was combined with 5-AzaC treatment (p<0.05).

These data demonstrate that in a fully methylated FXS cell line, demethylation of the locus resulted in expression of both FMR1 RNA isoforms. However, when demethylation was combined with an ASO against FMR1 isoform 12, an increase in the FMR1 isoform 1 mRNA was found. Thus, a combination of demethylation and ASO treatment may be useful for FXS patients with a fully methylated FMR1 locus.

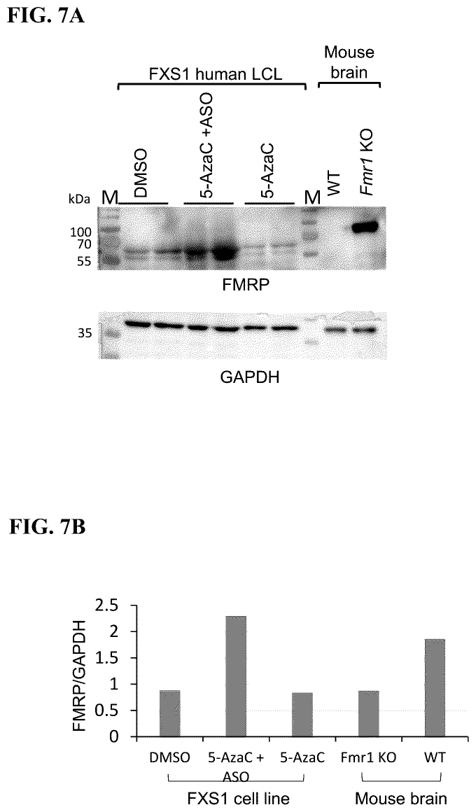

The upper panel of FIG. 7A shows western blot data for FXS1 LCL cell line in duplicates, demonstrating an increase in FMRP after treatment with 1 μM 5-AzaC and ASO treatment (80 nM of both antisense oligonucleotides 713 and 714) when compared to DMSO or 5-AzaC only treated samples. The mouse brains (hippocampus tissue) from a wild-type mouse and an Fmr1 knock-out mouse were loaded as controls. The FMRP protein from mouse tissues ran higher on the gel compared to the human FMRP. The bottom panel represents GADPH protein levels used to normalize the protein amounts loaded in each sample. FIG. 7B shows quantification of the FMRP protein levels relative to GAPDH protein levels as seen on the western blot in FIG. 7A.

These data demonstrate the FMRP protein levels from the samples analyzed for FMR1 RNA levels in FIGS. 6A-6B. Treatment of the FXS1 cell line (fully methylated FMR1 locus) with a demethylating agent (5-AzaC) alongside the ASO treatment against FMR1 iso12, resulted in a significant increase in FMRP protein levels as against the untreated FXS1 cells or the 5-AzaC treatment cells alone. As a comparison, the levels of FMRP protein expressed with this combination of treatment was similar to that seen in wild-type mouse brain tissues (see FIGS. 7A-7B).

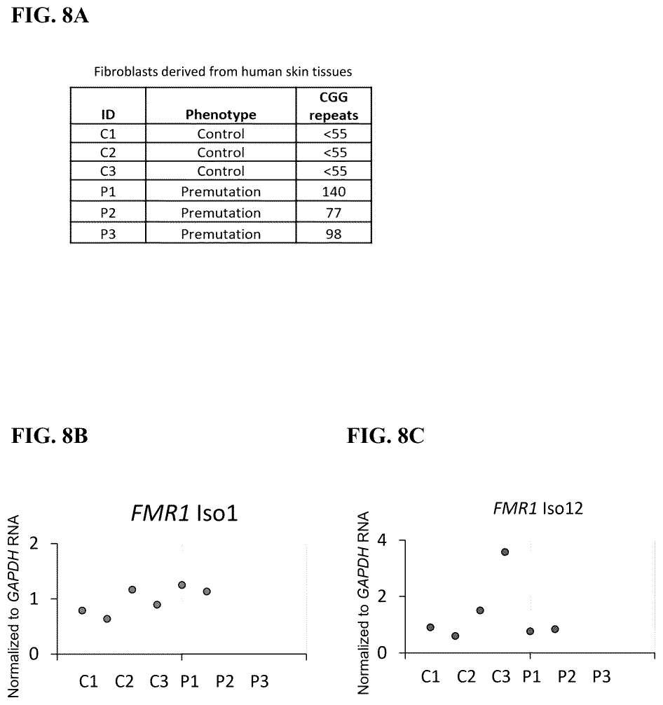

FIG. 8A is a table demonstrating the CGG repeats in the FMR1 RNA 5′ UTR from three healthy males and three premutation carrier males for FXS. The premutation carriers had 55-200 CGG repeats in the 5′UTR of FMR1 gene, whereas greater than 200 CGG repeats would lead to FXS, and less than 55 CGG repeats are usually present in healthy individuals. Premutation carriers have a propensity to develop FXTAS (Fragile X-associated tremor/ataxia syndrome) after the age of 50 yrs. FIG. 8B shows RT-qPCR data demonstrating the presence of similar FMR1 iso1 levels in fibroblast cells from all six individuals normalized to GAPDH RNA levels. FIG. 8C shows the presence of increased FMR1 iso12 levels in individual P1 compared to the other premutation carriers and healthy control samples. All premutation carriers expressed similar FMR1 iso1 levels as compared to the healthy controls. However, only individual P1 with higher CGG repeats (140, see FIG. 8A) expressed FMR1 iso12.

These data demonstrate that the FMR1 iso12 might be expressed in premutation carriers with a higher CGG repeat number, and, in some embodiments, ASO treatment in these individuals can be therapeutically beneficial by increasing FMRP protein levels.

### Prophetic Examples

In a first set of experiments, various ASOs will be introduced, singly or in combination, into human FXS WBC lines that are partially methylated and hence express some FMR1 RNA. At various time points, for example, about 24, 48, 72, 96, 120, 144 and 168 hours after transfection, levels of FMR1 iso1, FMR1 iso12, and FMRP will be assessed.

In a second set of experiments, human FXS WBC lines that have full methylation of FMR1 DNA and express no FMR1 RNA will be incubated with varying amounts of DNA demethylation agent, for example, 5-aza-2-deoxycytidine (5-azadC) (Sigma A3656), to partially demethylate the FMR1 DNA. Then, various ASOs will be introduced, singly or in combination, into the DNA demethylase-treated cells. At various time points, for example, about 24, 48, 72, 96, 120, 144 and 168 hours after transfection, levels of FMR1 iso1, FMR1 iso12, and FMRP will be assessed.

In a third set of experiments, various ASOs will be introduced, singly or in combination, into primary fibroblasts from FXS patients that are partially methylated. At various time points, for example, about 24, 48, 72, 96, 120, 144 and 168 hours after transfection, levels of FMR1 iso1, FMR1 iso12, and FMRP will be assessed. In the primary fibroblasts from patients with a completely methylated FMR1 locus, the cells will be incubated with varying amounts of DNA demethylation agent, for example, 5-aza-2-deoxycytidine (5-azadC) (Sigma A3656), to partially demethylate the FMR1 DNA. Then, various ASOs will be introduced, singly or in combination, into the DNA demethylase-treated cells.

### Example 5. Safety and Efficacy in an Animal Model

The safety and efficacy of ASO treatment will be determined in an animal model. Neural progenitor cells, derived from human FXS patients with partially methylated FMR1 and iso12 expression, will be injected into NOD-scid IL2Rγ^{null} mouse pups as described by Windrem et al., A Competitive Advantage by Neonatally Engrafted Human Glial Progenitors Yields Mice Whose Brains Are Chimeric for Human Glia, J Neurosci 34:16153-61 (2014) and Liu et al. Rescue of Fragile X syndrome neurons by DNA methylation editing of the FMR1 gene, Cell 172(5):979-92 (2018). Modified ASOs, such as those described above will be injected into the brain or via intraperitoneal injection (IP). The RNA will be extracted from the brains, and human FMR1 iso1 and iso12 will be quantified by RT-qPCR. This experiment will determine the safety and efficacy of ASO treatment in inhibiting FMR1 iso12 production and promoting iso1 formation in an animal model. FMRP in human neurons will be assessed by immunocytochemistry.

TABLE 1
Non-limiting Examples of ASOs and Other Pertinent Information.

| | SEQ | | Scale | nt | ε | MW | Vol | Conc. | | |
|---|---|---|---|---|---|---|---|---|---|---|
| Oligo # | ID NO | Sequence | (μMol) | Count | (L/mol*cm) | (g/mol) | (μL) | (μM) | μMol | nmol/μL |
| W-704 | 1 | AGAAGCCAAAG | 1 | 20 | 216990 | 8035.67 | 500 | 614.68 | 0.31 | 0.61 |
| | | GAGACCTGA | | | | | | | | |
| W-705 | 2 | AAAGAGAAGCC | 1 | 20 | 231300 | 8054.99 | 500 | 598.53 | 0.30 | 0.60 |
| | | AAAGGAGAC | | | | | | | | |
| W-706 | 3 | CTAGACCGGAAA | 1 | 22 | 236430 | 8832.38 | 500 | 663.28 | 0.33 | 0.66 |
| | | AGAGAAGCCA | | | | | | | | |
| W-707 | 4 | ATGCTAGACCGG | 1 | 21 | 233100 | 8439.7 | 500 | 582.75 | 0.29 | 0.58 |
| | | AAAAGAGAA | | | | | | | | |
| W-708 | 5 | CAATGCTAGACC | 1 | 20 | 214470 | 8010.4 | 500 | 610.06 | 0.31 | 0.61 |
| | | GGAAAAGA | | | | | | | | |
| W-709 | 6 | AAGTOCCAATGC | 1 | 21 | 205740 | 8384.38 | 500 | 561.92 | 0.28 | 0.56 |
| | | TAGACCGGA | | | | | | | | |
| W-710 | 7 | TCTCCGAAGTCC | 1 | 20 | 178560 | 7920.39 | 500 | 603.77 | 0.30 | 0.60 |
| | | CAATGCTA | | | | | | | | |
| W-711 | 8 | GAGCTCTCCGAA | 1 | 18 | 159390 | 7148.33 | 500 | 605.31 | 0.30 | 0.61 |
| | | GTCCCA | | | | | | | | |
| W-712 | 9 | AGAACAGTGGA | 1 | 20 | 196650 | 8007.03 | 500 | 617.65 | 0.31 | 0.62 |
| | | GCTCTCCGA | | | | | | | | |
| W-713 | 10 | CGCCCAGAACAG | 1 | 20 | 186120 | 7996.35 | 500 | 669.57 | 0.33 | 0.67 |
| | | TGGAGCTC | | | | | | | | |
| W-714 | 11 | CCTCGCOCAGAA | 1 | 20 | 186120 | 7996.35 | 500 | 576.78 | 0.29 | 0.58 |
| | | CAGTGGAG | | | | | | | | |

### Examples 6-10

Fragile X Syndrome (FXS) is a neuro-developmental disorder causing a range of maladies including intellectual disability, speech and developmental delays, social deficits, repetitive behavior, attention deficits, and anxiety. Previous studies have shown an expansion of >200 CGG triplets in the 5′UTR of Fragile X Messenger Ribonucleoprotein 1 (FMR1) induces gene methylation and transcriptional silencing, loss of the encoded FMRP, and FXS. Fragile X Messenger Ribonucleoprotein (FMRP) is an RNA-binding protein that interacts with >1000 mRNAs in the mouse brain and human neurons, predominantly through coding region associations (1-3). Although earlier studies suggested that FMRP inhibits protein synthesis (4), subsequent high-resolution methods showed that FMRP promotes as well as inhibits translation (5-8). One mechanism by which FMRP inhibits translation is stalling ribosome translocation on mRNAs (9, 10). Previously, several mRNAs associated with FMRP-stalled ribosomes were identified, one of which encodes SETD2, an epigenetic enzyme that trimethylates histone H3 lysine 36 (H3K36me3) (11). SETD2 was elevated in Fmr1-deficient hippocampus, which resulted in an altered H3K36me3 chromatin landscape. H3K36me3 resides in gene bodies and influences alternative pre-mRNA splicing (12), and indeed multiple mRNAs were mis-spliced in Fmr1-deficient mouse hippocampus. Many of these mis-splicing events were also detected in the human postmortem autism spectrum disorder (ASD) brain and blood tissues(14-18), indicating a convergence of FXS and ASD (11, 13).

Because mis-splicing of mRNAs is widespread in Fmr1-deficient mouse brain, and because individuals with FXS are often on the autism spectrum, it was surmised that RNA mis-splicing might also be prevalent in human FXS patient tissues (blood and brain). Accordingly, leukocytes were isolated from freshly obtained blood from 29 FXS males and 13 typically developing (TD) age-matched males, and RNA sequencing was performed. The analysis revealed widespread and statistically robust mis-regulation of alternative splicing and RNA abundance of greater than 1,000 mRNAs. Mis-regulated RNA expression and processing in FXS postmortem brain were also found.

Further analysis of the RNA-seq data unexpectedly revealed that FMR1 RNA was expressed in 21 of 29 FXS leukocyte samples, some nearly as high as FMR1 transcript levels from TD individuals. Because all FXS samples were from individuals with >200 CGG repeats, this was a surprising result because the FMR1 locus, which was purported to be silent under these conditions, was transcriptionally active in patients even when the gene appeared to be fully methylated in standard assays. However, the highest FMR1 RNA expressing FXS individuals were mosaic (CGG repeat number mosaicism or partial methylation of a full expansion). Furthermore, it was found that much of the FMR1 mRNA in the FXS individuals was itself mis-spliced to generate FMR1-217, a little-known 1.8 kb isoform comprised of FMR1 exon 1 and a pseudo-exon within FMR1 intron 1. This isoform is predicted to encode a truncated, 31 amino acid polypeptide whose function, if any, is unknown. Additional analysis revealed that FMR1-217 was detected in FXS dermal and lung-derived fibroblasts as well as in five of seven FXS postmortem cortex samples, further indicating the preponderance of FMR1 mis-splicing in FXS populations and, most importantly, that this altered processing event occurs in the brain as well as leukocytes. Fibroblasts from some FXS premutation (i.e., ˜55-200 CGG repeats) male carriers also expressed FMR1-217 as well as full-length FMR1 RNA, indicating that mis-splicing may be widespread in other disorders linked to CGG expansions in FMR1.

These findings suggest that modulation of FMR1 mis-splicing is a suitable approach to increase FMRP levels in individuals expressing FMR1-217. To investigate further, eleven 2′-O-methoxyethyl (MOE)/phosphorothioate-containing antisense oligonucleotides (ASOs) against several regions of FMR1-217 were generated and transfected into an established FXS lymphoblast cell line that expresses this transcript. Single ASOs or a combination of two ASOs blocked improper FMR1 splicing, rescued proper FMR1 splicing, and restored FMRP to TD levels. Moreover, application of the DNA methylation inhibitor 5-aza-2′-deoxycytidine (5-AzadC) to a second FXS lymphoblast line as well as FXS fibroblast lines that normally do not express any FMR1 resulted in synthesis of both FMR1 and FMR1-217 RNAs but little or no FMRP. However, treatment of these cells with both 5-AzadC and the ASOs produced strong FMRP up-regulation. These studies demonstrated that first, in cells from FXS but not TD individuals, a significant proportion of the FMR1 RNA was mis-spliced to produce the FMR1-217 isoform; and second, ASO treatment to reduce FMR1-217 levels resulted in FMRP restoration to TD levels. Therefore, ASO treatment may offer a novel therapeutic approach to mitigate FXS.

Aberrant alternative splicing of mRNAs results in dysregulated gene expression in multiple neurological disorders. Surprisingly, the Fragile X Messenger Ribonucleoprotein 1 (FMR1) gene was transcribed in >70% of the FXS tissues, in many instances even when the gene was fully methylated. In all FMR1 expressing FXS tissues, FMR1 RNA itself was mis-spliced in a CGG expansion-dependent manner to generate the little-known FMR1-217 RNA isoform, which is comprised of FMR1 exon 1 and a pseudo-exon in intron 1. FMR1-217 was also expressed in FXS premutation carrier-derived skin fibroblasts and brain tissue. It was shown that in cells aberrantly expressing mis-spliced FMR1, antisense oligonucleotide (ASO) treatment reduced FMR1-217, rescued full-length FMR1 RNA, and restored Fragile X Messenger Ribonucleoprotein (FMRP) to normal levels. Notably, FMR1 gene reactivation in transcriptionally silent FXS cells using 5-aza-2′-deoxycytidine (5-AzadC), which prevented DNA methylation, increased FMR1-217 RNA levels but not FMRP. ASO treatment of cells prior to 5-AzadC application rescued full-length FMR1 expression and restored FMRP. These findings indicate that in FXS individuals (e.g., those expressing FMR1-217), ASO treatment may offer a new therapeutic approach to mitigate the disorder.

### Example 6. Materials and Methods

### Human FXS Participant Studies

All participants were Caucasian males with a FMR1 full mutation (CGG repeats>200) or typically developing individuals (CGG repeats<55) as confirmed by DNA analysis. All participants or their legal guardians, as appropriate, signed informed consent to the study. The project was approved by the Rush University Medical Center Institutional Review Board. Intelligence quotient (IQ) scores were obtained using the Stanford-Binet Scale-Fifth Edition (SB5) (52) and applying the z-deviation method to avoid floor effects in persons with intellectual disability (53). The adaptive skills of participants were determined using an semi-structured interview and measured using the Vineland Adaptive Behavior skills (Vineland-3, (54)). The Adaptive Behavior Composite (ABC) standard score (SS) was the measure of overall adaptive functioning based on scores assessing the following domains: communication, daily living skills, and socialization. FXS patients were aged 16-38 years with FXS phenotypes, a z-deviation IQ range of 20-52 and ABC standard score range of 20-41. Age matched TD individuals for the study were aged 22-29 with a normal IQ and no known neuropsychiatric conditions. For CGG repeat size determination in the 5′ UTR of the FMR1 gene, DNA isolated from whole blood was analyzed using the Asuragen FMR1 AmplideX PCR Kit. Methylation status was determined using the Asuragen FMR1 methylation PCR Kit and/or Southern blot analysis. FMRP levels were quantified by generating dried blood spots (DBS) from the samples. To generate DBS, 12-50 μl spots were put on each blood card and allowed to dry. The blood cards were then stored at −80° C. Discs were punched using a 6-mm punch and incubated in lysis buffer. Extracted sample was centrifuged, and FMRP was quantified using the LUMINEX® Microplex immunochemistry assay. FMRP levels were normalized to 1,000 WBCs per sample. Additionally, FMRP levels were also quantified by using peripheral blood mononuclear cell (PBMC) samples. PBMCs were isolated from whole blood using Cell Preparation (CPT) blood tubes. Isolated PBMC were lysed and quantified for total protein concentration using a spectrophotometer, and FMRP was quantified using a LUMINEX® Microplex immunochemistry assay. FMRP levels were normalized to total protein. Both methods produced comparable levels of FMRP in the samples assessed.

Frozen post-mortem brain tissues were obtained from University of California at Davis Brain Repository from FXS male individuals (N=6) and age-matched typically developing (TD) males (N=5).

RNA Extraction and Sequencing of Tissue Samples from FXS and TD Individuals

### Leukocytes

Eight milliliters (mL) of fresh blood were collected from FXS male individuals (N=29) and age-matched typically developing (TD) males (N=13) in a BD VACUTAINER® Cell Preparation Tube (CPT, with sodium citrate-blue top tube, Becton Dickinson #REF362761), and the leukocytes were collected on a LeukoLOCK™ filter, prior to RNA extraction using a LeukoLOCK™ Fractionation & Stabilization Kit (Ambion #1933) as per the manufacturer's instructions. Briefly, the blood samples were passed through LeukoLOCK™ filters that were then rinsed with 3 mL of phosphate buffered saline (PBS), followed by 3 mL of RNALATER®. The residual RNALATER® was expelled from the LeukoLOCK™ filter, and the filters were capped and stored in −80° C. To extract RNA, the filters were thawed at room temperature for 5 minutes, and then the remaining few drops of RNAlater were removed. The filter was flushed with 4 mL of TRIZOL® LS Reagent (ThermoFisher Scientific #10296028), and the lysate was collected in a 15-mL tube. 800 μl bromo-3-chloro-propane (BCP) (Sigma #B9673) was added to each tube and vortexed vigorously for 30 seconds. The tube was then incubated at room temperature for 5 minutes and centrifuged for 10 minutes at 4° C. at ˜2,000×g; the aqueous phase containing the RNA was recovered. To recover the long RNA fraction, 0.5 volume of 100% ethanol was added and mixed well. The RNA was then recovered using an RNA clean and concentrator kit (Zymo Research, #11-325/R1015), DNase-treated with TURBO™ DNase (Invitrogen #AM2238), resuspended in RNase-free water, and stored at −80° C. The quality of RNA (RNA integrity number (RIN)>7.3) was assessed using a 5300 Fragment Analyzer instrument. Three milligrams (mgs) of RNA sample were used for directional mRNA library preparation using polyA enrichment (Novogene Co), and the libraries were sequenced on the NovaSeq platform to generate paired end, 150-bp reads at a sequencing depth of 60-90 million reads per sample.

### Brain Tissue

The post-mortem frozen cortical tissues from FXS male individuals (N=6) and age-matched typically developing (TD) males (N=5) were powdered in liquid nitrogen using a mortar and pestle. The fine powder was then homogenized on ice in a Dounce homogenizer using TRIZOL® Reagent (ThermoFisher Scientific #15596026), and the lysates were collected. Total RNA was extracted using BCP, recovered as described above, and stored at −80° C.

cDNA Synthesis and qPCR

One microgram (μg) of total RNA was primed with oligo(dT)₂₀ to generate cDNA with a QuantiTect cDNA synthesis kit (Qiagen, #205311) using random hexamers (Table 3). qPCR was performed using the iTaq™ Universal SYBR® Green Supermix (BIO-RAD #1725122) on a QuantStudio 3 qPCR machine in duplicate.

### RNA-Seq Data Analysis

FASTQ files were uploaded to the DolphinNext platform (55) at the UMass Chan Medical School Bioinformatics Core for mapping and quantification. The reads were subjected to FastQC (v0.11.8) analysis, and the quality of reads was assessed. Reads were mapped to the genome assembly GRCh38 (hg38) version 34 using the STAR (v2.5.3a) aligner. Gene and isoform expression levels were quantified by salmon v1.5.2.

Differential gene expression analysis: DESeq2 (v3.9) was used to obtain differentially expressed genes from the estimated counts table. After normalization by the median of ratios method, genes with minimal 5 counts average across all samples were kept for the Differential Gene expression analysis. P<0.0002 was used as a cutoff. The TDF files generated were uploaded on the Integrative Genomics Viewer (2.6.2) and autoscaled for visualization.

Alternative splicing analysis: To analyze differential alternative splicing (AS), the rMATS package v3.2.5 (14) was used with default parameters. The Percent Spliced In (PSI) levels or the exon inclusion levels were calculated by rMATS using a hierarchical framework. To calculate the difference in PSI between genotypes, a likelihood-ratio test was used. AS events with an FDR<5% and |deltaPSI|≥5% as identified using rMATS were used for further analysis. The genes with significant skipped exons were used for validation using RT-qPCR analysis. One μg of RNA was used to generate cDNA using the QuantiTect cDNA synthesis kit. Primers were designed to overlap skipped/inclusion exon junctions, and qPCR was performed using the Bio-Rad SYBR reagent on a Quantstudio3 instrument.

### Cell Culture

### Lymphoblast Cell Lines

Lymphoblast cell lines (LCL) were obtained from Coriell Institute from two FXS individuals (GM07365 (FXS1), GM06897(FXS2)) and two typically developing control males (GM07174 (WT3), GM06890 (WT4)). Cells were cultured in RPMI 1640 medium (Sigma-Aldrich), supplemented with 15% fetal bovine serum (FBS) and 2.5% L-glutamine, at 37° C. with 5% CO₂ in T25 flasks.

### Fibroblast Cells

Skin biopsies from participants were collected in a 15-cc tube with transfer culture medium (DMEM with 5% Gentamicin). The biopsy was then removed from the transfer medium with tweezers onto a sterile tissue culture dish and dissected into approximately 6-7 pieces using sterile tweezers and scissors in the culture hood. Three to four pieces of skin explants were kept on the bottom of a T25 flask, and 3 mL CHANG AMNIO culture medium was added. The flask was then incubated at 37° C. with 5% CO₂ for 10 days. The culture medium was changed after cells started growing out from the skin explants. After the cells had grown to 5-6 layers around the skin explants, the skin explants were removed from the culture flask, and fibroblasts were trypsinized and spread evenly in the flask. The media were changed after overnight incubation with trypsin. Fibroblast culture medium was added (complete medium (500 mL DMEM (15-017-CV) with 10% FBS and 1×antibiotic-antimitotic, 5 mL 1×L-glutamine)) twice a week to cells in a T25 culture flasks at 37° C. with 5% CO₂.

Fibroblast cell lines were obtained from Coriell Institute from two FXS individuals (GM05131, and GM07072). A control fibroblast line derived from a skin sample of a typically developing male was used. Cells were cultured in DMEM medium (Sigma-Aldrich), supplemented with 10% fetal bovine serum (FBS) and 2.5% L-glutamine, at 37° C. with 5% CO₂.

### ASO Synthesis and Treatment

### ASO Synthesis

ASOs were synthesized on a Dr. Oligo 48 synthesizer. 2′-O-methoxyethyl (MOE)-modified phosphoramidites were coupled for 8 minutes. Oligonucleotides were deprotected in concentrated aqueous ammonia (30% in water) at 55° C. for 16 hours and characterized by liquid chromatography-mass spectrometry. Final desalting was effected by diafiltration (3× water wash) in a 3-kDa cutoff Amicon centrifugal filter.

### ASO Treatment

Antisense oligonucleotides (ASOs) were dissolved in ultrapure distilled water to a final concentration of 10 μM. Before use, the ASOs were heated to 55° C. for 15 minutes and cooled at room temperature. ASOs were added, individually or in combinations, to LCL cell lines at a final concentration of 80 nM or 160 nM using Lipofectamine RNAIMAX® Transfection Reagent (Thermo Fisher Scientific, 13778030) and incubated at 37° C. with 5% CO₂ for 16 hours in reduced serum medium. RPMI 1640 medium (Sigma-Aldrich), supplemented with 15% fetal bovine serum (FBS) was added for a total of 72 hours. The cells were collected after 72 hours of ASO treatment for RNA and protein extraction.

### 5-AzadC Treatment

For each cell culture, 30×10⁵ cells/mL were added to a final volume of 20 mL medium (RPMI 1640 medium (Sigma-Aldrich) supplemented with 15% fetal bovine serum (FBS) and 2.5% L-glutamine at 37° C. with 5% CO₂) per T25 flask. 5-Aza-2′-deoxycytidine (5-AzadC) (Sigma-Aldrich, A3656) was added to the cell cultures (final concentration 1 μM) for 7 consecutive days. A 2 mM stock of 5-AzadC was made in DMSO. For each cell line, two independent treatments were performed (n=2). For the no treatment controls for each cell line, DMSO was added to the flasks. For samples with both 5-AzadC and ASO treatment, 80 nM or 160 nM ASOs or vehicle were added on Day 1 and either 5-AzadC or DMSO was added each day from Day 2 up to Day 9 at a final concentration of 1 μM. On Day 9 the cells were collected in 1× phosphate buffered saline to proceed with RNA extraction or Western blotting.

### Western Blotting

Cells were homogenized at 4° C. in RIPA buffer, with incubation on ice for 10 minutes and dissociation by pipetting. The extract was centrifuged at 13,200 rpm for 10 minutes at 4° C., and the supernatant collected. Protein concentration was determined using BCA reagent. Proteins (10 μg) were diluted in SDS-bromophenol blue reducing buffer with 40 mM DTT and analyzed using western blotting with the following antibodies: FMRP (Millipore, mAb2160, 1: 1,000), FMRP (Abcam, ab17722, 1:1,000) and GAPDH (14C10, Cell Signaling Technology, mAb 2118, 1:2,000), diluted in 1×TBST with 5% non-fat milk. Membranes were washed three times for 10 minutes with 1×TBST and incubated with anti-rabbit or anti-mouse secondary antibodies (Jackson, 1:10,000) at room temperature for 1 hour. Membranes were washed three times for 10 minutes with 1×TBST, developed with ECL-Plus (Piece), and scanned with GE Amersham Imager.

### Quantification and Statistical Analysis

All grouped data were presented as mean±s.e.m. All tests used to compare the samples were mentioned in the respective figure legends and corresponding text. When exact P values were not indicated, they were represented as follows: *, p<0.05; **, p<0.01; ***, p<0.001; ****, P value<0.0001; n.s., p>0.05.

### Data and Code Availability

Codes and scripts used for quantification analysis were written in Python or R and will be provided upon request. Data Resources Sequencing datasets generated in this study have been deposited into the Gene Expression Omnibus (GEO) database under the accession number: Super series GSE202179. The sub series GSE202177 comprise the raw data for the RNA-seq and GSE202178 for the ChIP-Seq experiments.

Chromatin immunoprecipitation Sequencing (ChIP-Seq)

Eight mL of fresh blood was collected from FXS male (N=10) and age-matched typically developing males (N=7) individuals in a BD VACUTAINER® CPT (Cell Preparation Tube with sodium citrate-blue top tube, Becton Dickinson #REF362761). The tube was gently inverted 5 times, and the sample was centrifuged for 25 minutes at 1,500-1,800 RCF at room temperature. The tubes were then inverted to collect the lymphocytes and other mononuclear cells resuspended in the upper liquid phase in a new 15-mL tube. The samples were centrifuged again for 10 minutes at 300 RCF to obtain the PBMC pellet. The PBMCs were rinsed with 1× Dulbecco's phosphate buffered saline without calcium or magnesium (D-PBS) (Invitrogen #14190-094). The PBMC pellet was resuspended in 250 μL ice-cold D-PBS with protease inhibitors. FMRP levels in PBMCs were quantified using a LUMINEX® Microplex immunochemistry assay. Chromatin isolation and sequencing were performed as previously described (11). Briefly, the cells were cross-linked with 1% formaldehyde and quenched with 150 mM glycine. After centrifugation at 2,000 g for 10 minutes at 4° C., the cells were lysed. After homogenization, the nuclei were harvested by centrifugation at 2,000 g for 5 minutes at 4° C. The nuclei were lysed by incubating for 20 minutes on ice in nuclear lysis buffer (10 mM Tris (pH 8.0), 1 mM EDTA, 0.5 mM EGTA). 0.5% SDS was added, and the samples were sonicated on a Bioruptor® sonicator at high power settings (sonication: 30 seconds on, 90 seconds off) for 9 cycles of 15 minutes each at 4° C. The samples were centrifuged and diluted to adjust the SDS concentration to <0.1%. 10% of each sample was used as input. The remainder of the samples were divided into two and incubated with protein G DYNABEADS® coupled overnight at 4° C. with antibodies against H3K36me3 (Abcam ab9050, 5 g per ChIP) or H3K4me3 (Active Motif-39159, 5 μg per ChIP). After IP, the beads were washed, and chromatin de-crosslinked overnight at 65° C. After RNase and proteinase K treatment, the DNA was purified. ChIP-Seq libraries were prepared by performing the following steps: ends repair using T4 DNA polymerase, A′ base addition by Klenow polymerase, and Illumina adapter ligation using T4 Polynucleotide kinase from New England Biolabs (NEB). The library was PCR amplified using multiplexing barcoded primers. The libraries were pooled with equal molar ratios, denatured, diluted, and sequenced with NextSeq 500/550 High Output Kit v2.5 (Illumina, 75-bp paired-end runs) on a Nextseq500 sequencer (Illumina).

### ChIP-Seq Analysis

For ChIP-seq data analysis, alignments were performed with Bowtie2 (2.1.0) using the GRCh38 (hg38) version 34 genome, duplicates were removed with Picard and TDF files for Genomics Viewer (IGV), viewing were generated using a ChIP-seq pipeline from DolphinNext (55). The broad peaks for H3K36me3 ChIP-Seq were called using the broad peak parameter MACS2. Narrow peaks for H3K4me3 ChIP were called using the narrow parameter in MACS2. deepTools2 (57) was used to plot heatmaps and profiles for genic distribution of H3K36me3 and H3K4me3 ChIP signals over input. IGV tools (2.6.2) were used for visualizing TDF files, and all tracks shown were normalized for total read coverage.

### Example 7. FMR1 RNA is Expressed and Mis-Spliced in a Subset of FXS Individuals

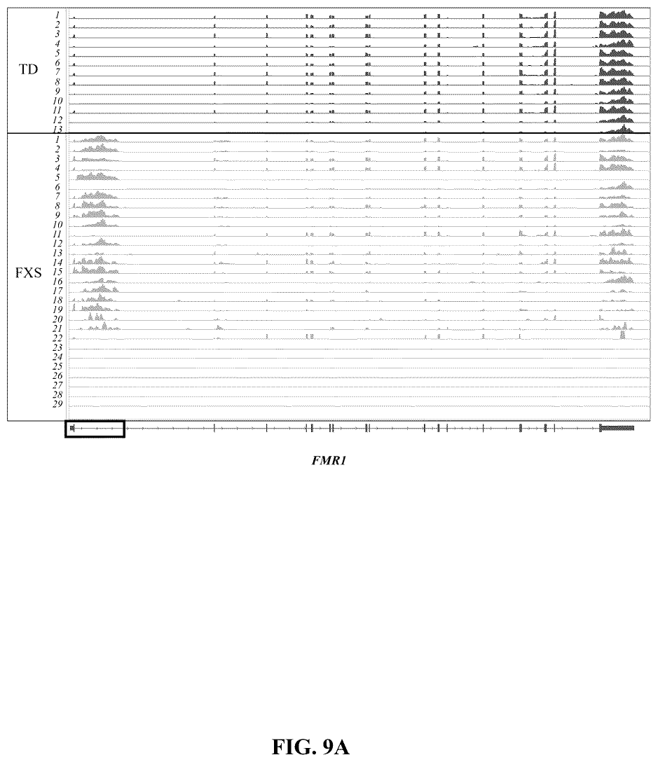

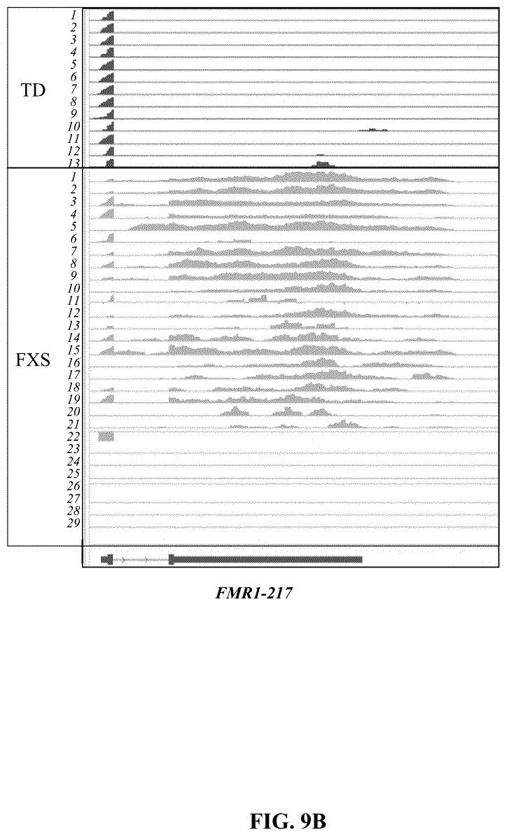

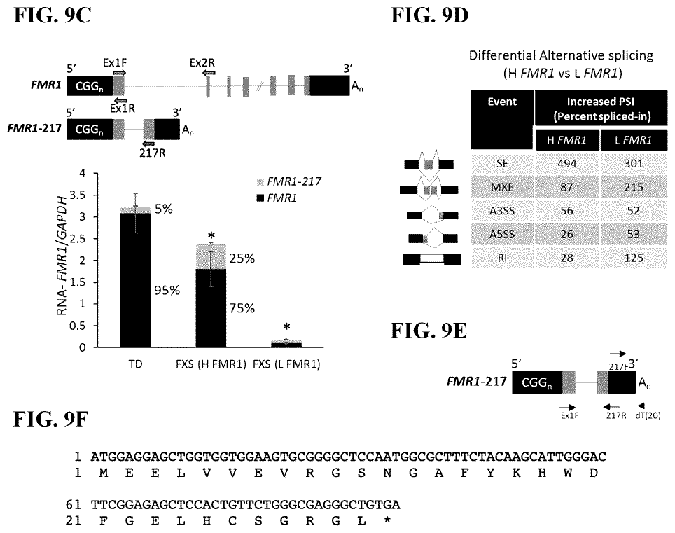

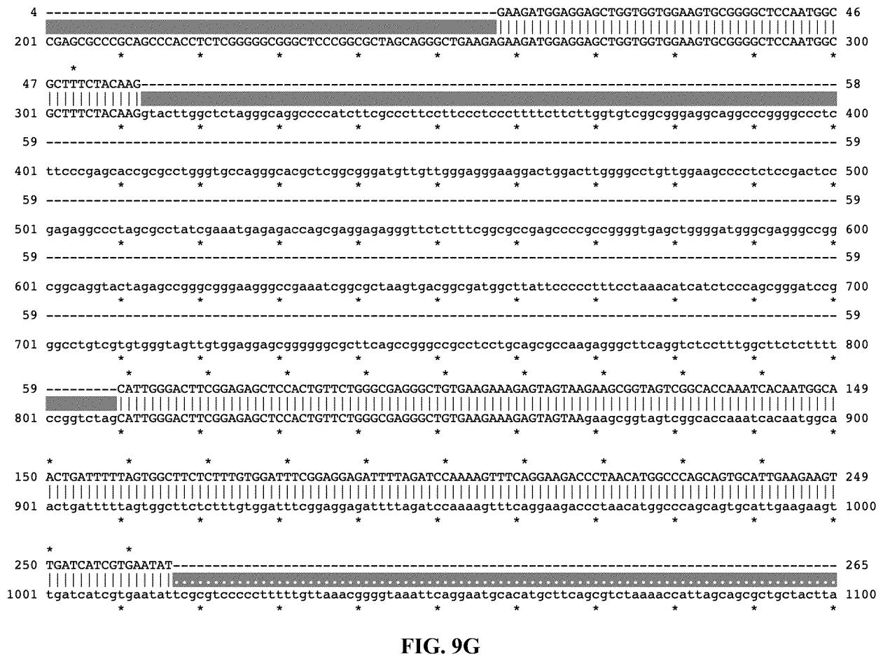

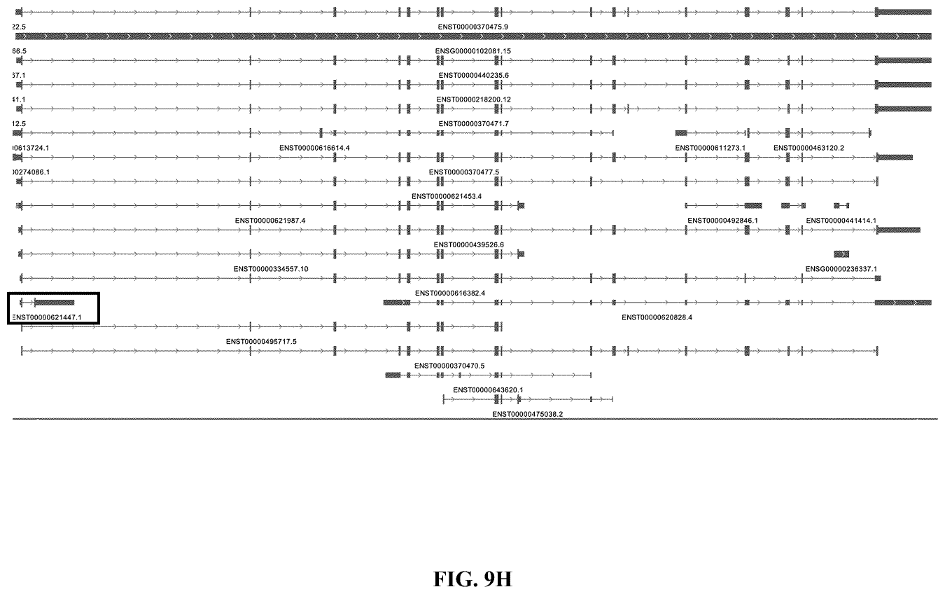

Expansion of >200 CGG repeats in FMR1 induces gene methylation, transcriptional silencing, loss of FMRP, and FXS. It was therefore surprising that in leukocytes of 21 of 29 FXS individuals, FMR1 RNA was detected, and in four individuals, the level of all isoforms of this RNA were similar to, or even higher than, those in the TD individuals (Table 2, FMR1 RNA TPM levels). When only full-length FMR1 encoding 632 amino acid FMRP (FMR1-205) was examined (FIG. 9H, Table 3), WBCs from 6 individuals had levels of this transcript that were similar to those of TD (Table 2). For comparison, the levels of the FMR1 paralog FXR2 were similar in all individuals (Table 2). Visualizing the RNA reads at the FMR1 locus with the Integrated Genome Viewer (IGV) made it evident that exonic reads were detected at robust levels in TD individuals, and that the exonic reads were also detected in FXS individuals (FIGS. 9A-9B). FXS individuals 1-21 expressed relatively high FMR1 levels (with a cutoff of 0.6 transcript per million (TPM)) (H FMR1), compared to FXS individuals 22-29 who expressed low or undetectable FMR1 levels (L FMR1) (Table 2 and FIGS. 9A-9B). Remarkably, the H-FMR1 FXS individuals displayed strong RNA reads in intron 1 of FMR1 (thick-lined black box in FIG. 9A, enlarged in FIG. 9B). Notably, RNA reads in this intronic region were not detected in any TD individuals even though FMR1 RNA was strongly expressed (FIGS. 9A-9B). The FMR1 locus expresses multiple alternatively spliced RNA isoforms (Table 3). The RNA reads detected in FMR1 intron 1 correspond to the second exon of the FMR1-217 RNA isoform. FMR1-217 (ENST00000621447.1) is a 1.8-kb transcript comprised of two exons, and is predicted to encode a 31-amino acid polypeptide (Table 3). Notably, most of the total FMR1 RNA in the FXS samples was comprised of the aberrantly spliced FMR1-217 transcript, which was absent in samples from TD individuals (Table 2). TPMs of all 14 FMR1 isoforms detected in the TD and FXS patient samples were obtained (data not shown). RT-PCR was used to detect the FMR1-217 isoform in the FXS leukocyte samples (reverse transcription primed with oligodT(20)), and the amplified product was sequenced using primers specific to the FMR1-217 exon-exon junction. Aligning this sequence to FMR1 confirmed that this transcript is polyadenylated and is a spliced product of FMR1 exon 1 and FMR1-217 exon 2 (FIG. 9C).

TABLE 2
FMR1 RNA TPM levels

| Sample | FMR1 | FMR1-205 | FMR1-217 | FXR2 |
|---|---|---|---|---|
| TD1 | 31.1 | 1.9 | 0.1 | 17.0 |
| TD2 | 26.3 | 3.7 | 0.1 | 15.5 |
| TD3 | 23.7 | 2.6 | 0.2 | 14.0 |
| TD4 | 23.0 | 1.6 | 0.1 | 9.0 |
| TD5 | 22.1 | 1.3 | 0.1 | 14.3 |
| TD6 | 20.6 | 1.7 | 0.2 | 12.4 |
| TD7 | 19.5 | 1.9 | 0.1 | 12.1 |
| TD8 | 18.8 | 2.6 | 0.1 | 9.4 |
| TD9 | 18.4 | 0.8 | 0.0 | 7.4 |
| TD10 | 16.2 | 1.1 | 0.0 | 12.7 |
| TD11 | 15.7 | 1.9 | 0.1 | 8.2 |
| TD12 | 13.6 | 2.2 | 0.1 | 14.0 |
| TD13 | 12.6 | 0.3 | 0.1 | 9.0 |
| FXS1 | 36.2 | 3.2 | 18.6 | 10.1 |
| FXS2 | 32.6 | 1.7 | 24.4 | 10.6 |
| FXS3 | 28.5 | 2.0 | 10.6 | 15.4 |
| FXS4 | 17.0 | 0.8 | 2.7 | 11.7 |
| FXS5 | 10.9 | 0.0 | 10.5 | 11.5 |
| FXS6 | 8.4 | 0.5 | 0.2 | 10.2 |
| FXS7 | 8.0 | 0.4 | 5.9 | 17.0 |
| FXS8 | 4.9 | 0.0 | 2.9 | 14.0 |
| FXS9 | 4.2 | 0.0 | 2.8 | 11.1 |
| FXS10 | 3.8 | 0.0 | 2.7 | 12.4 |
| FXS11 | 3.8 | 0.0 | 0.1 | 12.1 |
| FXS12 | 2.9 | 0.1 | 2.3 | 12.4 |
| FXS13 | 2.9 | 0.0 | 0.6 | 15.2 |
| FXS14 | 2.2 | 0.2 | 1.3 | 14.4 |
| FXS15 | 2.1 | 0.1 | 1.5 | 12.8 |
| FXS16 | 2.0 | 0.0 | 0.9 | 10.8 |
| FXS17 | 1.6 | 0.0 | 1.1 | 12.4 |
| FXS18 | 1.1 | 0.0 | 0.8 | 13.0 |
| FXS19 | 1.0 | 0.0 | 0.7 | 15.5 |
| FXS20 | 0.6 | 0.0 | 0.4 | 6.4 |
| FXS21 | 0.6 | 0.2 | 0.3 | 9.4 |
| FXS22 | 0.0 | 0.0 | 0.0 | 15.7 |
| FXS23 | 0.0 | 0.0 | 0.0 | 8.4 |
| FXS24 | 0.0 | 0.0 | 0.0 | 16.1 |
| FXS25 | 0.0 | 0.0 | 0.0 | 10.5 |
| FXS26 | 0.0 | 0.0 | 0.0 | 12.0 |
| FXS27 | 0.0 | 0.0 | 0.0 | 10.6 |
| FXS28 | 0.0 | 0.0 | 0.0 | 15.2 |
| FXS29 | 0.0 | 0.0 | 0.0 | 13.3 |

Table 2 shows normalized gene counts (transcripts per million, TPM) obtained from RNA-seq data analysis for total FMR1 (all isoforms), FMR1-205 (encoding the full-length, 632 amino acid FMRP), FMR1-217 (a mis-spliced RNA), and FXR2, a paralogue of FMR1.

TABLE 3
FMR1 Transcript Identification & Corresponding Predicted Amino Acid
Numbers of Encoded Proteins from ENSEMBL (56)

| Transcript ID | Name | bp | Protein | Biotype |
|---|---|---|---|---|
| ENST00000370475.9 | FMR1-205 | 4441 | 632aa | Protein coding |
| ENST00000690137.1 | FMR1-226 | 4166 | 615aa | Protein coding |
| ENST00000218200.12 | FMR1-201 | 4333 | 611aa | Protein coding |
| ENST00000691111.1 | FMR1-228 | 4154 | 599aa | Protein coding |
| ENST00000687593.1 | FMR1-223 | 4159 | 594aa | Protein coding |
| ENST00000439526.6 | FMR1-207 | 3699 | 592aa | Protein coding |
| ENST00000370470.5 | FMR1-203 | 1774 | 590aa | Protein coding |
| ENST00000690216.1 | FMR1-227 | 4008 | 587aa | Protein coding |
| ENST00000440235.6 | FMR1-208 | 4271 | 586aa | Protein coding |
| ENST00000370477.5 | FMR1-206 | 3437 | 582aa | Protein coding |
| ENST00000686086.1 | FMR1-222 | 3995 | 570aa | Protein coding |
| ENST00000691214.1 | FMR1-229 | 4067 | 569aa | Protein coding |
| ENST00000495717.6 | FMR1-212 | 2874 | 561aa | Protein coding |
| ENST00000685491.1 | FMR1-221 | 4109 | 559aa | Protein coding |
| ENST00000621453.5 | FMR1-218 | 1827 | 548aa | Protein coding |
| ENST00000370471.7 | FMR1-204 | 4125 | 537aa | Protein coding |
| ENST00000616382.5 | FMR1-214 | 2799 | 536aa | Protein coding |
| ENST00000692108.1 | FMR1-232 | 4252 | 509aa | Protein coding |
| ENST00000689517.1 | FMR1-224 | 4484 | 460aa | Protein coding |
| ENST00000693512.1 | FMR1-235 | 3402 | 398aa | Protein coding |
| ENST00000334557.10 | FMR1-202 | 1295 | 297aa | Protein coding |
| ENST00000621987.5 | FMR1-219 | 2440 | 297aa | NMD |
| ENST00000616614.4 | FMR1-215 | 1409 | 76aa | NMD |
| ENST00000693452.1 | FMR1-234 | 4093 | 49aa | NMD |
| ENST00000692091.1 | FMR1-231 | 3908 | 49aa | NMD |
| ENST00000475038.3 | FMR1-209 | 2747 | 49aa | NMD |
| ENST00000621447.1 | FMR1-217 | 1832 | 31aa | Protein coding |
| ENST00000691793.1 | FMR1-230 | 5731 | | RI |
| ENST00000492846.2 | FMR1-211 | 5650 | | RI |
| ENST00000689570.1 | FMR1-225 | 5650 | | RI |
| ENST00000620828.4 | FMR1-216 | 4830 | | RI |
| ENST00000693079.1 | FMR1-233 | 4647 | | RI |
| ENST00000643620.1 | FMR1-220 | 1439 | | RI |
| ENST00000611273.1 | FMR1-213 | 564 | | RI |
| ENST00000478848.1 | FMR1-210 | 541 | | RI |

Next, the proportion of full-length FMR1 RNA to FMR1-217 RNA in TD or FXS leukocytes was assessed. In the TD samples, 95% of the total FMR1 RNA (primers Ex1F and Ex1R) represented full-length molecules (primers Ex1F and Ex2R), whereas in the H FMR1 samples, 75% of the total FMR1 RNA was full-length and 25% was FMR1-217 (primers Ex1F and 217R) (FIG. 9C). In the L FMR1 samples, both isoforms were just barely detected. The total FMR1 RNA levels in all the samples were normalized to GAPDH RNA expression (* denotes P values<0.05). Importantly, all FXS individuals in this study, irrespective of FMR1 expression, displayed typical FXS symptoms, suggesting that even in patients with high FMR1 expression, functional FMRP may not be present or is present at very low amounts (FMRP protein levels were quantified for available samples (data not shown)).

Whether stratification of FXS individuals, based on relatively high (H) or low (L) amounts of FMR1 (using a cutoff of 0.6 TPM, Table 2), was reflected in transcriptome-wide RNA changes was examined. By reanalyzing FXS leukocyte RNA-seq data to compare significant RNA alterations between these two groups, hundreds of aberrant splicing events that tracked with the amount of this mis-spliced transcript were found (FIG. 9D and data not shown). Whether the parameters measured in WBCs correlated with intelligence quotient (IQ) was investigated. Table 4 presents determinations of methylation status of the FMR1 gene (by PCR), FMRP levels (ng/g protein), CGG repeat number, FMR1-217, full-length FMR1-205, all detected FMR1 isoforms and IQ (Stanford-Binet test).

TABLE 4
Characterizing Leukocytes of Each FXS Individual

| | | | | | | FMR1- | FMR1- |
|---|---|---|---|---|---|---|---|
| Lab | CGG repeat | | PBMC [ng FMRP/ | | FMR1 | 205 | 217 |
| ID | number | Methylation (MPCR) | μg total protein] | IQ | (TPM) | | |
| FXS01 | 140, 175, >200 | 140 100%, 175 | 6.56E−03 | 37.8 | 36.2 | 3.2 | 18.6 |
| | | 97%, >200 90% | | | | | |
| FXS02 | >200 | 81% | 2.07E−03 | 26.8 | 32.6 | 1.7 | 24.4 |
| FXS03 | 150, >200 | N/A | N/A | 52.0 | 28.5 | 2.0 | 10.6 |
| FXS04 | 102, >200 | N/A | N/A | 37.0 | 17.0 | 0.8 | 2.7 |
| FXS05 | >200 | >200 96% | 4.85E−04 | 35.1 | 10.9 | 0.0 | 10.5 |
| FXS06 | 65, >200 | 65 98%, >200 100% | 1.77E−02 | 55.0 | 8.4 | 0.5 | 0.2 |
| FXS07 | >200 | N/A | 2.28E−04 | 25.0 | 8.0 | 0.4 | 5.9 |
| FXS08 | 173, >200 | N/A | N/A | 39.9 | 4.9 | 0.0 | 2.9 |
| | (~710, ~613) | | | | | | |
| FXS09 | >200 | 100% | 4.40E−04 | 35.0 | 4.2 | 0.0 | 2.8 |
| FXS10 | >200 | 100% | 3.11E−04 | 56.0 | 3.8 | 0.0 | 2.7 |
| FXS11 | >200 | 100% | 6.50E−03 | 62.3 | 3.8 | 0.0 | 0.1 |
| FXS12 | >200 | 100% | 4.85E−04 | 26.9 | 2.9 | 0.1 | 2.3 |
| FXS13 | 102, 174, >200 | 102, 174 | 5.35E−04 | 27.6 | 2.9 | 0.0 | 0.6 |
| | | 100%, >200 100% | | | | | |
| FXS14 | >200 | N/A | N/A | 20.0 | 2.2 | 0.2 | 1.3 |
| FXS15 | >200 | 100% | 2.56E−04 | 45.9 | 2.1 | 0.1 | 1.5 |
| FXS16 | 63, >200 | 63.98%, 194 | 3.50E−03 | 53.5 | 2.0 | 0.0 | 0.9 |
| | | 36%, >200 100% | | | | | |
| FXS17 | >200 | 100% | 1.05E−04 | 44.0 | 1.6 | 0.0 | 1.1 |
| FXS18 | >200 | N/A | 1.34E−04 | 30.3 | 1.1 | 0.0 | 0.8 |
| FXS19 | >200 | N/A | N/A | 50.0 | 1.0 | 0.0 | 0.7 |
| FXS20 | >200 | 100% | 4.85E−04 | 29.6 | 0.6 | 0.0 | 0.4 |
| FXS21 | >200 | 100% | 2.00E−04 | 37.7 | 0.6 | 0.2 | 0.3 |
| FXS22 | >200 | N/A | 4.88E−04 | 35.8 | 0.0 | 0.0 | 0.0 |
| FXS23 | >200 | 94% | N/A | N/A | 0.0 | 0.0 | 0.0 |
| FXS24 | 28**, >200 | 100% | N/A | 37.6 | 0.0 | 0.0 | 0.0 |
| FXS25 | >200 | 100% | N/A | N/A | 0.0 | 0.0 | 0.0 |
| FXS26 | >200 | 100% | 4.85E−04 | 41.8 | 0.0 | 0.0 | 0.0 |
| FXS27 | >200 | >200 100% | 4.85E−04 | 20.2 | 0.0 | 0.0 | 0.0 |
| FXS28 | >200 | N/A | N/A | N/A | 0.0 | 0.0 | 0.0 |
| FXS29 | >200 | >200: 85% | N/A | 49 | 0 | 0 | 0 |

In Table 4, FMR1 gene methylation (MPCR): in percent as determined by methylation PCR (MPCR) analysis; FMRP levels: ng/μg total protein; FMR1: all isoforms; IQ: Stanford-Binet; N/A: not available.

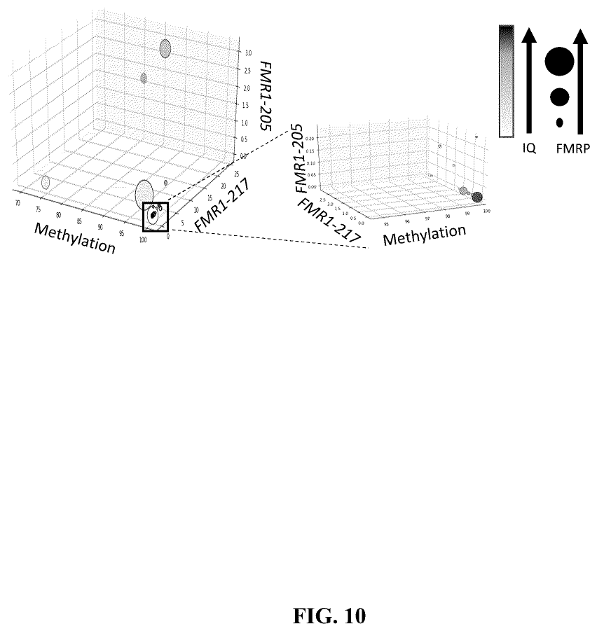

Table 5 presents correlation coefficients for pairwise comparisons of the measurements noted above. Methylation of the FMR1 gene is negatively correlated with FMR1-217 and FMR1-205 expression. More intriguing is the moderately positive correlation of IQ with FMRP protein levels. Somewhat surprisingly, FMR1-205, which encodes full-length FMRP, has no correlation with IQ. However, it is noted that while FMR1-205 encodes the complete 632-amino acid FMRP, other FMR1 isoforms, which vary in abundance, encode truncated FMRP proteins (Table 3). Without presupposing functionality of truncated FMRP proteins, the canonical FMR1 isoform, FMR1-205, was used for further comparisons. FMR1-217 has a negative correlation with IQ, indicating a deleterious effect of this isoform. FIG. 10 displays a 3-dimensional comparison of all the parameters noted above. The inset shows that some FXS patients with a fully methylated FMR1 gene expressed FMR1 RNA and FMRP. Taken together, these results show several important findings. First, the FMR1 locus is frequently transcribed even when the FMR1 gene with a full CGG expansion is fully methylated. Second, FMRP levels in WBCs are positively correlated with IQ. Third, the negative correlation of FMR1-217 with IQ suggests that the process of mis-splicing, the 31-amino acid polypeptide derived from FMR1-217, the FMR1-217 RNA itself, or a combination thereof (e.g., all three), impart some toxic effect manifest in the brain (e.g., IQ). In any event, the levels of FMR1-217 expression, as well as additional transcriptome-wide changes in RNA processing events, likely form the basis for molecular stratification of FXS individuals.

TABLE 5
Correlation coefficients for pairwise comparisons for indicated parameters

| | Methylation | FMRP | IQ | FMR1 | FMR1-205 | FMR1-217 |
|---|---|---|---|---|---|---|
| Methylation | 1.0 | −0.2 | 0.3 | −0.9 | −0.8 | −1.0 |
| FMRP | −0.2 | 1.0 | 0.5 | 0.3 | 0.3 | 0.0 |
| IQ | 0.3 | 0.5 | 1.0 | −0.2 | −0.1 | −0.3 |
| FMR1 | −0.9 | 0.3 | −0.2 | 1.0 | 0.9 | 1.0 |
| FMR1-205 | −0.8 | 0.3 | −0.1 | 0.9 | 1.0 | 0.8 |
| FMR1-217 | −1.0 | 0.0 | −0.3 | 1.0 | 0.8 | 1.0 |

In Table 5, ±0-0.1: no correlation; 0.1-0.29: weak correlation; ±0.3-0.49: moderate correlation; ±0.5-1: strong correlation.

### Example 8. FMR1-217 is Expressed in Human FXS and Pre-Mutation Carrier Postmortem Brain

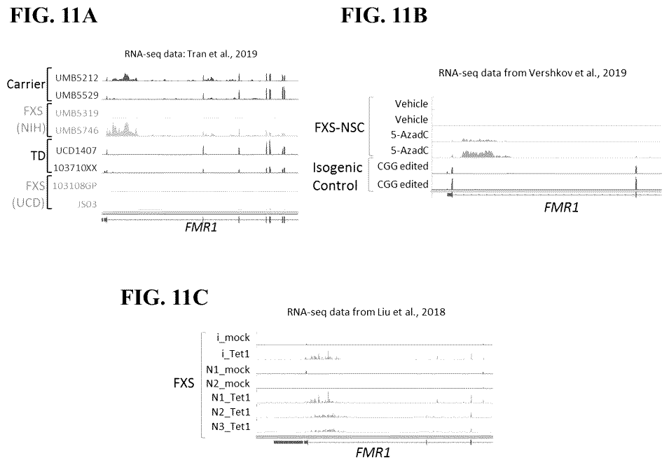

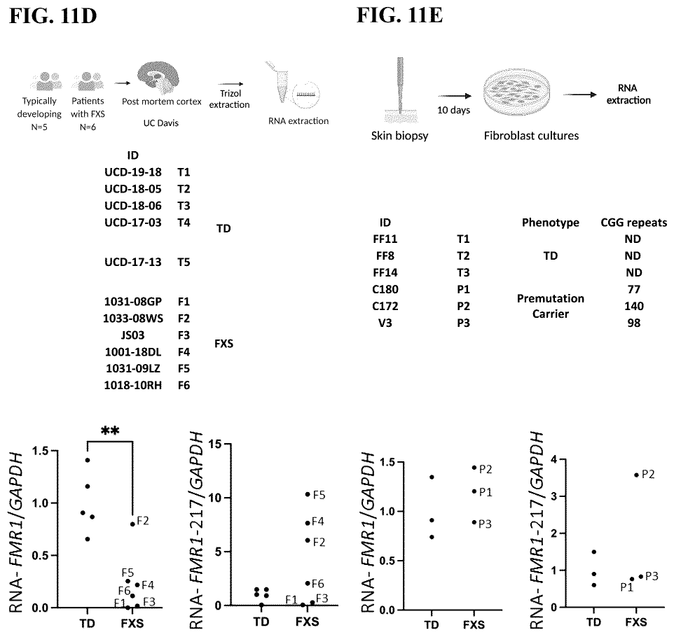

To investigate whether FMR1-217 is expressed in FXS brain, publicly available RNA-seq data of post-mortem frontal cortex tissues from FXS individuals (CGG repeats>200), FXS carriers (CGG repeats 55-200), and TD individuals (CGG repeats<55) (16) were analyzed. FMR1 RNA (TPM) levels were highest in pre-mutation carriers (Table 6). Interestingly, the FXS sample UMB5746, which displayed CGG repeat number mosaicism, displayed high levels of FMR1 RNA (Table 6 and FIG. 11A) and to a lesser extent, FMRP (16). The analysis showed that this individual expressed FMR1-217, as did FXS carrier UMB5212, who had Fragile X-associated tremor/ataxia syndrome (FXTAS) (Table 6 and FIG. 11A). Neither TD individual had any RNA reads corresponding to FMR1-217 (Table 6 and FIG. 11A). Thus, FMR1-217 RNA may only be expressed in the brains of a subset of FXS individuals and premutation carriers.

TABLE 6

| Sample | | | | FMR1- | FMR1- |
|---|---|---|---|---|---|
| repository | | Patient ID | FMR1 | 205 | 217 |
| NIH | Carrier | UMB5212 | 20 | 1.4 | 3.9 |
| NeuroBioBank | | UMB5529 | 23 | 1.6 | 0.4 |
| | FXS | UMB5319 | 0 | 0.0 | 0.0 |
| | | UMB5746 | 19 | 0.0 | 10.1 |
| UC Davis | TD | UCD1407 | 10 | 0.7 | 0.0 |
| FXTAS | | 103710XX | 12 | 1.5 | 0.1 |
| (UCD) | FXS | 103108GP | 0 | 0.0 | 0.0 |
| | | JS03 | 1 | 0.0 | 0.1 |

Table shows sample information or postmortem FXS frontal cortex, premutation FXS carriers and TD individuals (derived from (16)). RNA-seq datasets GSE107867 (NIH samples) and GSE117776 were reanalyzed for DGE and DAS. The TPM for FMR1 RNA in the samples is shown.

A BLAST analysis showed that FMR1-217 aligned only with intron 1 of FMR1 and with no other region of the genome. Additional data showed unequivocally that FMR1-217 is derived from FMR1, and that its synthesis is dependent the CGG expansion in this gene. Vershkov et al. (17) used CRISPR/Cas9 to delete the CGG expansion from FMR1 in FXS iPSC-derived neural stem cells (NSCs). Additional FXS NSCs were incubated with 5-AzadC, a nucleoside analogue that prevents DNA methylation. RNA sequencing from these samples, as well as from FXS NSCs incubated with vehicle, was then performed. The RNA-seq data from Vershkov et al. (17) was reanalyzed, some of which is presented in FIG. 11B, and FMR1 transcript quantification (TPM) in Table 7. RNA-seq reads corresponding to FMR1-217 were clearly evident in the FXS-NSCs incubated with 5-AzadC, but not in the other samples. Moreover, the CGG edited cells, which were isogenic to the unedited FXS NSCs, had no FMR1-217 reads, but instead robust expression of full-length FMR1. Quantification of the RNA-seq reads (TPM) showed strong total FMR1 and FMR1-205 expression in the CGG-edited and 5-AzadC-treated cells but not in vehicle-treated cells. More importantly, strong FMR1-217 expression was observed only in the 5-AzadC-treated cells. Therefore, FMR1-217 is derived from the FMR1 locus and requires a CGG expansion.

TABLE 7
FMR1 (Total, −205 or −217) reads (TPM)
of the samples in FIG. 11B

| | FMR1 | FMR1-205 | FMR1-217 |
|---|---|---|---|
| Vehicle | 0.0 | 0.0 | 0.0 |
| Vehicle | 0.2 | 0.0 | 0.0 |
| 5-AzadC | 9.9 | 0.8 | 6.9 |
| 5-AzadC | 6.1 | 1.9 | 3.9 |
| CGG edited | 27.1 | 3.1 | 0.1 |
| CGG edited | 33.0 | 7.6 | 0.3 |

In a complementary study, Liu et al. (18) performed a targeted FMR1 gene demethylation experiment by incubating FXS iPSC and FXS iPSC-derived neurons with a FMR1 small guide RNA and a catalytically inactive Cas9 fused to Tet1 demethylase sequences. Reanalysis of the subsequent RNA-seq data is shown in FIG. 11C, and FMR1 transcript quantification (TPM) in Table 8. Their experimental paradigm showed that FMR1-217 sequences were evident only when the gene was demethylated in the FXS cells. Quantification of the relevant transcripts in Table 8 showed that strong FMR1 and FMR1-205 expression was detected in the Tet1-treated samples (but inexplicably, no FMR1-205 in sample N1_Tet1), and FMR1-217 expression in all Tet1-treated samples. These data therefore show once again that FMR1-217 is derived from the FMR1 locus and requires a CGG expansion.

TABLE 8
FMR1 (Total, 205 or 217) reads (TPM)
of the samples in FIG. 11C.

| | FMR1 | FMR1-205 | FMR1-217 |
|---|---|---|---|
| i_mock | 0.1 | 0.0 | 0.0 |
| i_Tet1 | 69.9 | 0.0 | 7.3 |
| N1_mock | 0.1 | 0.0 | 0.0 |
| N2_mock | 0.1 | 0.0 | 0.0 |
| N1_Tet1 | 46.4 | 0.0 | 6.9 |
| N2_Tet1 | 81.3 | 22.7 | 13.3 |
| N3_Tet1 | 50.4 | 12.3 | 10.6 |

To confirm expression of FMR1-217 RNA in FXS brain tissue, frozen post-mortem cortex samples were obtained from six FXS males and five age-matched typically developing (TD) males (UC Davis Health). Using RT-qPCR, it was found that the FMR1 full-length RNA was significantly reduced in the FXS individuals compared to that in the TD individuals. However, 3 or 4 of the 6 FXS individuals expressed varying levels of the FMR1 full-length RNA as well as FMR1-217 RNA (1031-09LZ, 1001-18DL and 1033-08WS) (FIG. 11D). Previous studies on the FXS sample 1031-09LZ had noted expression of FMR1 RNA similar to that in TD individuals, despite the presence of a methylated fully mutated FMR1 locus (19). However, no detectable FMRP was found in the FXS brain sample 1031-09LZ (20). Also, in agreement with these studies, RNA-seq data from Tran et al. showed no FMR1 RNA in the FXS tissue samples (1031-08GP and JS03) (Table 6 and FIG. 11A) as well as an absence of FMRP (16).

FMR1-217 RNA was detected in only one of the two premutation carrier samples. To gain greater insight into the relationship of FMR1-217 FXS carrier tissue (CGG repeats between 55-200), skin biopsies were obtained from 3 additional premutation carriers and 3 TD individuals (FIG. 11E). The skin samples were cultured in vitro to generate fibroblast cell lines for RNA analysis. Interestingly, using RT-qPCR, FMR1-217 was detected in one premutation carrier (C172) with 140 CGG repeats but not in samples with 77 or 98 CGG repeats (FIG. 11E). There was no change in total FMR1 RNA levels among the samples (FIG. 11E). Thus, generation of FMR1-217 may be linked to the number of CGG repeats in the FMR1 gene.

### Example 9. FMR1-217 RNA is Expressed in Lymphoblast Cell Cultures from FXS Individuals

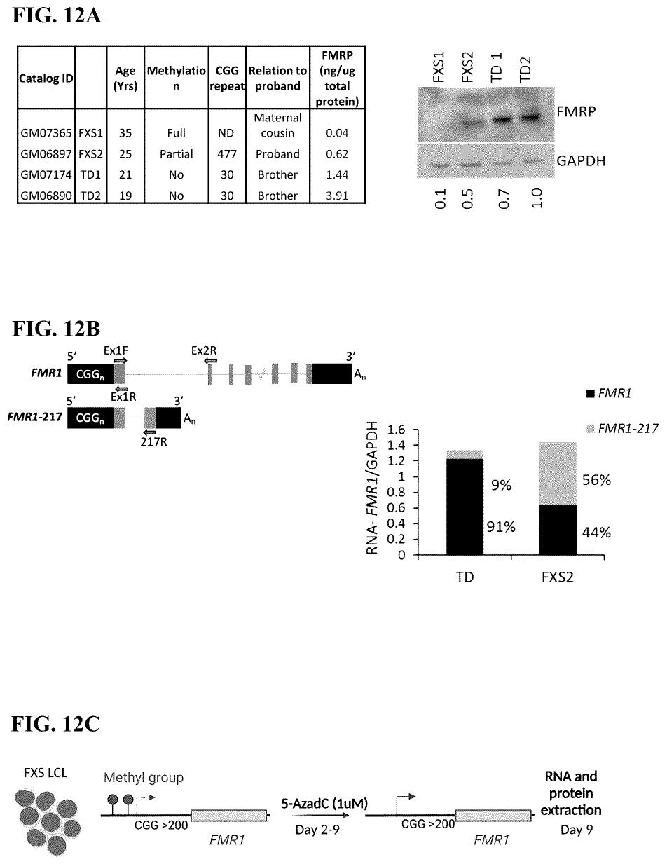

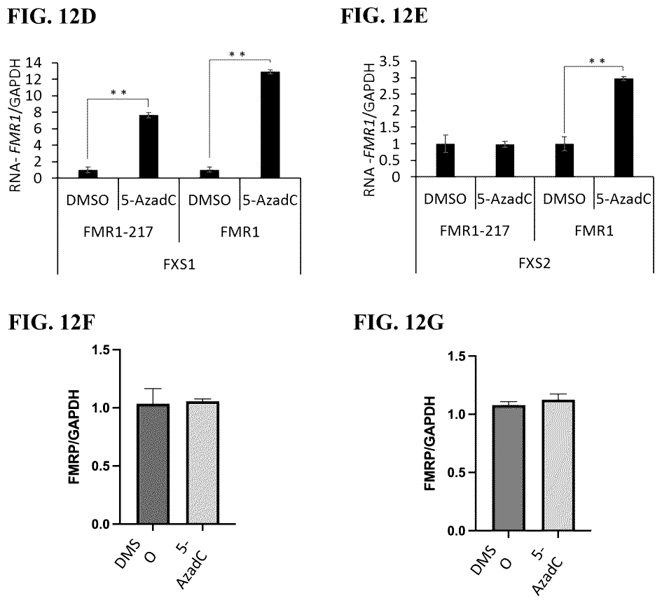

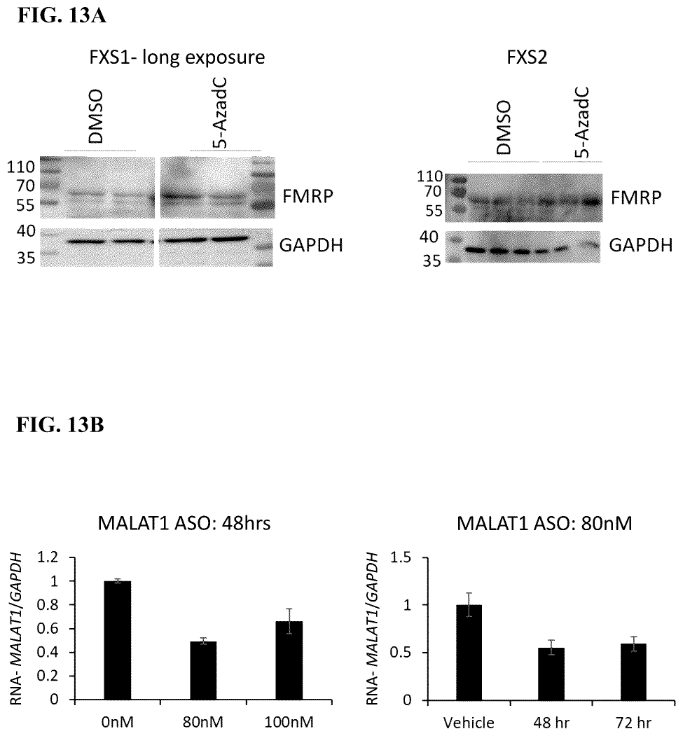

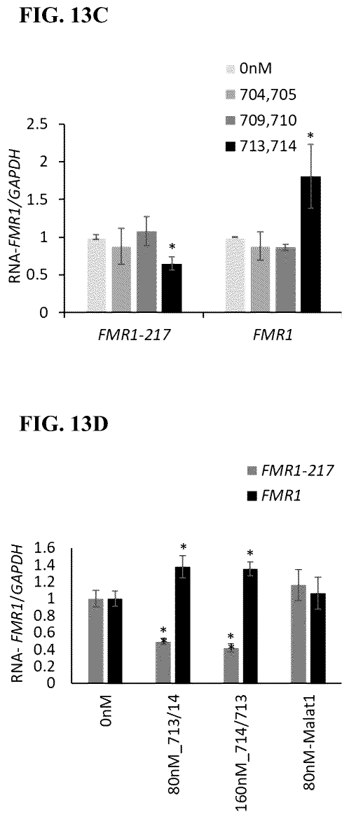

DNA methylation of the CpG island upstream of the FMR1 gene promoter in FXS individuals (MFM, methylated full mutation) contributes to transcriptional silencing of the locus and loss of FMRP. FMR1 transcription can be reactivated by treatment with the nucleoside analogue 5-AzadC (5-aza-2′-deoxycytidine), which inhibits DNA methylation (21, 22). Consequently, whether re-activating FMR1 transcription in cells from FXS individuals with a completely silenced and presumably fully methylated FMR1 locus results in FMR1-217 expression was investigated. For these experiments, lymphoblast cell lines (LCLs) derived from a FXS individual with a fully methylated locus (MFM) that was transcriptionally inactive (FXS1, GM07365), a FXS individual with a presumably partially methylated locus (UFM) that expressed some FMR1 RNA (FXS2, GM06897), and two typically developing individuals (TD1, GM07174, and TD2, GM06890), were used (all samples from Coriell Institute, NJ, USA) (FIG. 12A). Western blot analysis showed that modest levels of FMRP were detected in FXS2, but not FXS1 cell lines. FMRP was strongly expressed in TD1 and TD2 cells (ratios of FMRP/GAPDH relative to TD2 were shown below the blot) (FIG. 12A). Similar ratios of FMRP protein expression in these cell lines were obtained by the LUMINEX® Microplex immunochemistry assay (FMRP levels in ng FMRP/g total protein) (FIG. 12A). Using RT-qPCR, it was found that FMR1-217 RNA is expressed in FXS2 LCLs and comprises 56% of the total FMR1 RNA compared to only 9% in TD cells (FIG. 12B). It is noteworthy that although total FMR1 RNA levels in FXS2 cells were similar to those in TD cells, FMRP levels were much lower (FIGS. 12A-12B). Next, FXS1 and FXS2 cell lines were treated with 5-AzadC, and then FMR1 RNA and FMRP levels were measured (FIG. 12C). In the FXS1 cell line, treatment with 5-AzadC for seven days resulted in significant increases of both full-length FMR1 and FMR1-217 RNAs relative to DMSO-treated cells (FIG. 12D). However, in FXS2 cells, 5-AzadC treatment resulted in an increase of only full-length FMR1 RNA (FIG. 12E). In neither cell line did 5-AzadC treatment induce a significant increase in FMRP, suggesting either a longer treatment time or a higher concentration of 5-AzadC may be needed to induce FMRP expression (FIGS. 12F-12G and FIG. 13A). However, previous studies showed that longer treatment (36 days) of FXS LCLs with 5-AzadC restored FMR1 RNA only up to 40% and produced an even lower level FMRP compared to that in TD cells (22). Thus, transcriptional activation of normally silenced FMR1 by demethylation induces expression of full-length FMR1 and FMR1-217 RNAs but does not commensurately induce FMRP expression.

### Example 10. ASOs Targeting FMR1-217 Restored FMRP Levels in FXS LCLs with Partial or Complete FMR1 Gene Methylation

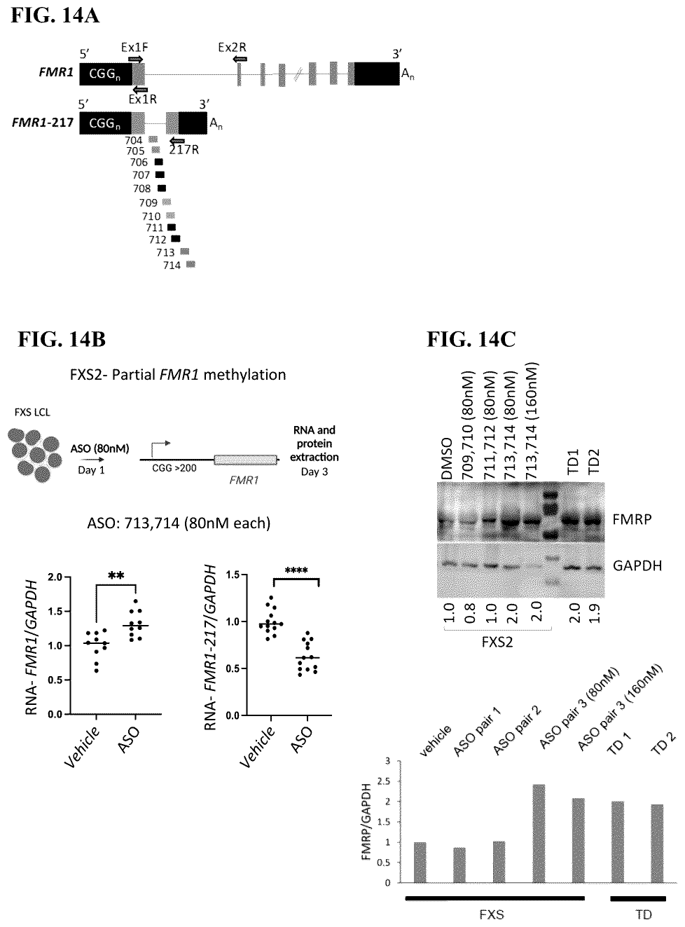

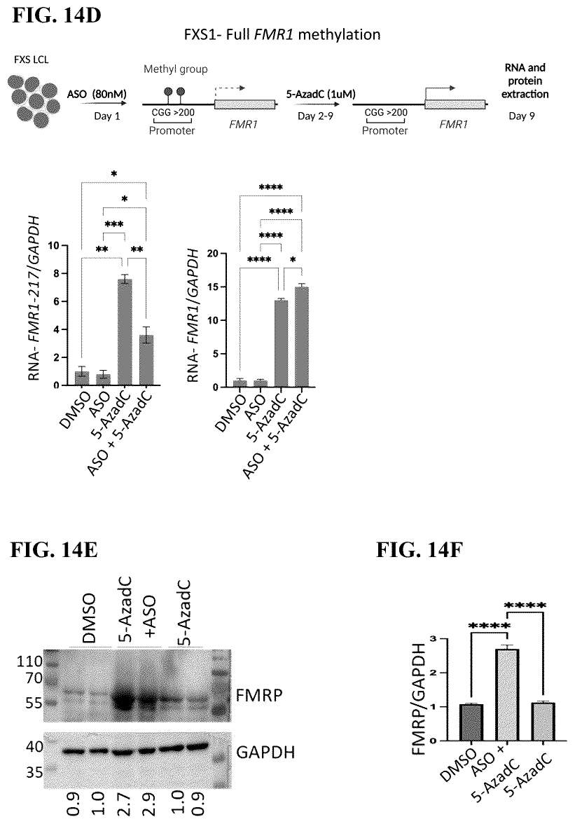

FMR1-217 was expressed in the UFM (partially methylated) FXS2 cells and after demethylation of MFM (fully methylated) FXS1 cells. At the time points tested, although full-length FMR1 increased in both FXS LCLs after 5-AzadC treatment, FMRP was unchanged. To test whether blocking the formation of FMR1-217 could lead to an increase in full-length FMR1 and concomitantly an increase in FMRP, 11 2′-O-methoxyethyl (MOE)-modified antisense oligonucleotides (ASOs) tiling across intron 1, the intron 1-exon 1 junction, or within exon 2 of FMR1-217 RNA were generated (FIG. 14A). First, an ASO targeting MALAT1 RNA (23) was used in LCL cultures to optimize treatment conditions and serves as a marker of transfection efficiency. LCLs cultured with 80 nM MALAT1 ASO for 72 hours led to ˜60% decrease in MALAT1 RNA levels (FIG. 13B), confirming that the transfection conditions were appropriate. Among the ASOs tested in FXS2 (FIG. 13C), the combination of ASO 713 and 714 (80 nM each) led to a significant decrease in FMR1-217 and an increase in full-length FMR1 (FIG. 14B, FIGS. 13C-13D). ASOs 713 and 714, at 80 nM or 160 nM each, for 72 hours elicited similar decreases in FMR1-217 and increases in full-length FMR1 RNA (FIG. 13D). The MALAT1 ASO had no effect on FMR1 isoform levels (FIG. 13D). Next, whether FMRP was restored in FXS2 cells following ASO treatment was assessed. FIG. 14C shows that 80 nM or 160 nM of ASOs 713 and 714 completely restored FMRP when compared to TD levels. Therefore, ASO treatment of cells from at least certain FXS individuals, which suggests a possible therapeutic path forward through FMRP restoration.

In the fully methylated FXS1 LCL, a 7-day treatment with 5-AzadC resulted in the expression of FMR1 and full-length FMR1 but did not affect FMRP levels. Thus, whether treatment of FXS1 LCLs with a combination of 5-AzadC and ASOs (713 and 714) could restore FMRP was addressed. FXS1 LCLs were incubated with 80 nM each of ASO 713 and 714, 24 hours preceding the addition of 1 μM of 5-AzadC every day for seven days prior to sample collection (FIG. 14D). FMR1 RNA isoform expression and FMRP levels were tested in these samples. Treatment with 5-AzadC alone led to the expected increase in FMR1 full length and FMR1-217 RNA compared to the DMSO control (FIG. 14D). Also, treatment with the ASOs alone did not affect FMR1 isoform levels, because the locus was completely methylated. However, treatment of cells with a combination of 5-AzadC and the ASOs rescued FMR1-217 RNA levels and further increased the full-length FMR1 compared to 5-AzadC treatment alone (FIG. 14D). Although FMRP levels were unaffected by 5-AzadC alone, FMRP was restored after treatment with a combination of 5-AzadC and the ASOs (FIGS. 14E-14F). These data showed that in FXS patient-derived cells with a UFM, treatment with FMR1-217 targeting ASOs restored FMRP levels while in MFM cells, a combinatorial treatment of demethylation (5-AzadC treatment) and ASOs restored FMRP.

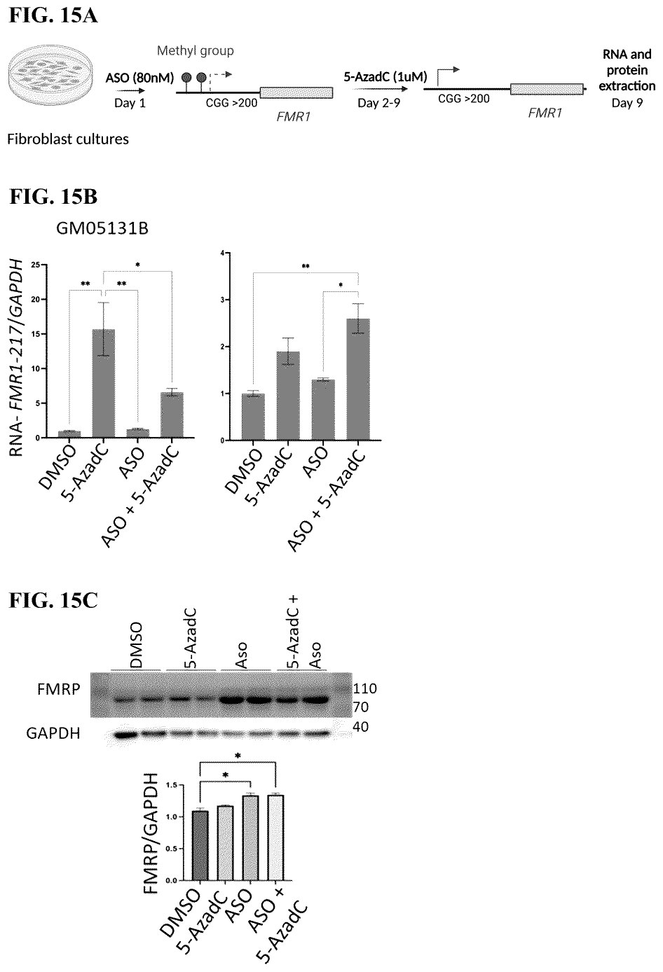

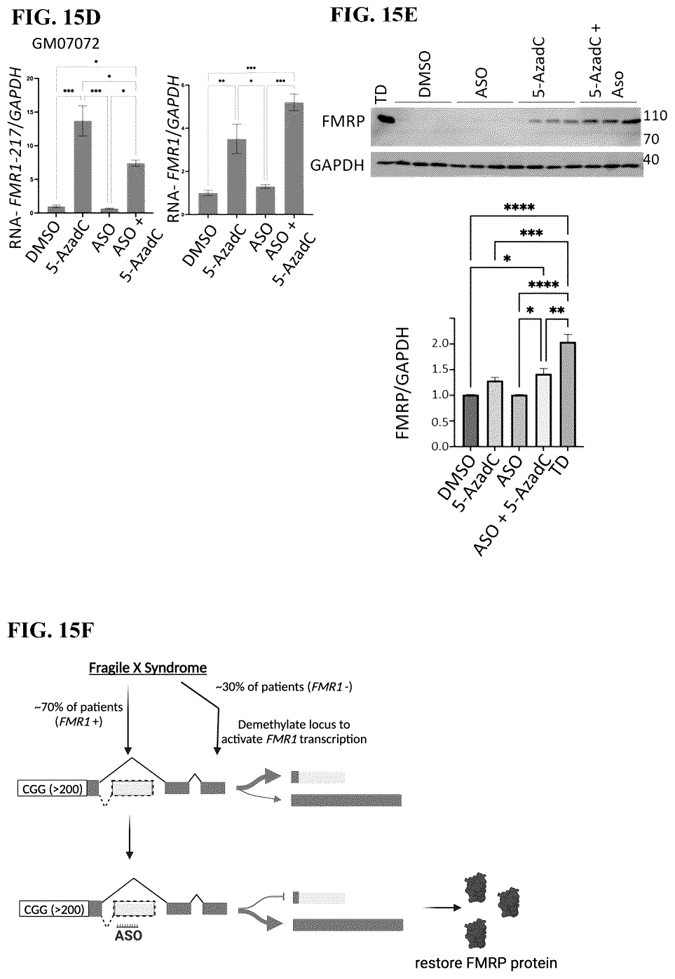

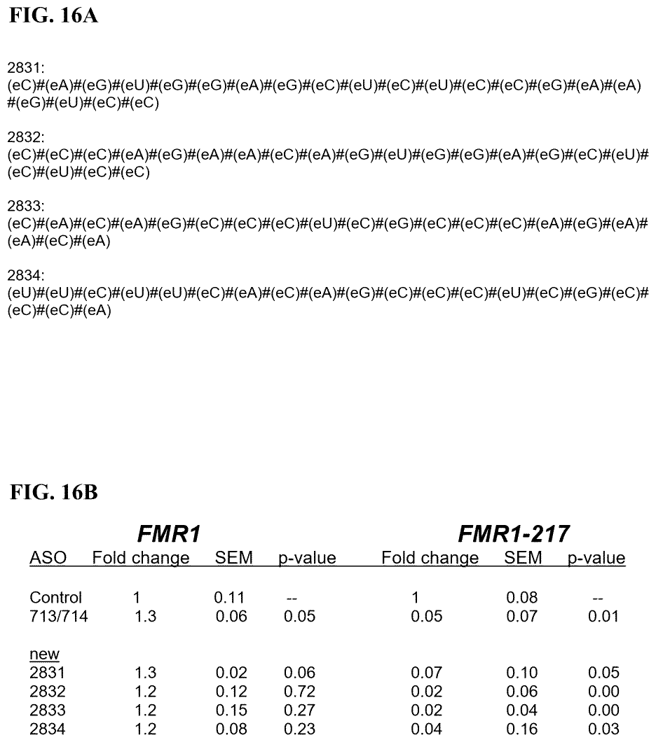

Finally, two FXS patient-derived fibroblast cell lines were incubated with 5-AzadC and the ASOs to determine FMR1 splicing rescue as well as restoration of FMRP. A dermal cell line from a FXS individual (5131b) with CGG repeat numbers of 800,166 (24), and previously shown to harbor a transcriptionally active FMR1 locus, was treated with 5-AzadC and then ASOs 713/714 for 72 hours before RNA and protein extraction (FIG. 15A). RT-qPCR of FMR1 and FMR1-217 showed an ASO-dependent decrease in FMR1-217 and a subsequent increase in FMR1 levels (FIG. 15B). The western blot in FIG. 15C showed while 5-AzadC treatment had no effect on FMRP levels, the ASOs alone or in combination with 5-AzadC significantly increased FMRP levels. In a similar experiment with lung fibroblasts from another FXS individual with a fully methylated FMR1 locus, incubation with 5-AzadC in the absence or presence of ASOs 713/714 resulted in increased FMR1 and FMR1-217 (FIG. 15D). The western blot in FIG. 15E showed, as with the dermal fibroblasts, ASO treatment resulted in a significant increase of FMRP, albeit lesser than that in the TD fibroblast line.

To summarize, it was found that in most FXS patient samples tested, the FMR1 locus was active but predominantly expressed a mis-spliced FMR1-217 isoform as well as very modest levels of FMRP. In the FXS cells that are transcriptionally silent, application of demethylating agents induced FMR1 transcription, which resulted in FMR1-217 expression. In both cases, treatment of cells with ASOs to block FMR1-217 production resulted in partial to complete restoration of FMRP (FIG. 15F).

Defects in alternative splicing of mRNAs alter the transcript and protein repertoire of cells and occur in many neurological disorders such as autism, schizophrenia, and bipolar disorder (25-27). In fragile X syndrome model (e.g., Fmr1 knockout) mice, hundreds of dysregulated alternative splicing events were detected, a number of which appeared to be linked to an altered epigenetic histone H3 lysine 36 trimethylation (H3K36me3) landscape (11). In this study, >1000 RNA mis-splicing events were detected in human FXS white blood cells, but interestingly, they do not correlate with H3K36me3, which is unaffected in FXS blood. The large number of white blood cell RNA changes, if correlated with certain pathologies of FXS, may be useful as biomarkers to assess therapeutic outcomes, disease prognosis, and cognitive abilities (28-30). Unlike protein-based biomarkers for FXS (31-33), blood derived RNA biomarkers are more sensitive and specific and can easily be translated into the clinic.

When it contains an expansion of 200 or more CGG repeats, the FMR1 gene promoter is methylated and transcriptionally silenced. It was therefore surprising that FMR1 RNA was detected in 19 of 29 FXS blood samples and in 5 of 10 FXS post-mortem brain samples. Most of these FXS individuals appeared fully mutated with >200 CGG repeats and methylated in standard assays. Remarkably, in >70% of these FXS cells and tissues, the FMR1 RNA was also mis-spliced to generate the FMR1-217 isoform, a highly truncated RNA that could encode a 31 amino acid peptide. FMR1-217 RNA was not detected in any TD sample. Moreover, in FXS individuals with a fully methylated and silenced FMR1 locus, abrogation of DNA methylation by 5-AzadC treatment results in FMR1-217 expression. FMR1 mis-splicing to generate the FMR1-217 isoform in FXS clearly requires a CGG expansion, although some evidence suggests that CGG repeat number may be a critical determinant for mis-splicing. For example, FMR1-217 RNA expression was detected in FXS premutation carrier-derived fibroblasts with 140 CGG repeats, but not lesser amounts (77 or 98 CGG repeats) or cells from TD individuals (<55 CGG repeats).

An important point is the non-linear relationship between FMR1 levels and FMRP expression in FXS tissue samples. The data show that although total FMR1 levels are similar in UFM FXS2 LCLs to that of the TD LCLs, FMRP expression is much lower. Likewise, high FMR1 expression does not ensure proper FMRP levels in FXS brain tissue samples 1031-09LZ and UMB5746 (16, 20). Similarly, in FXS LCLs and fibroblasts treated with 5-AzadC, a robust increase in FMR1 RNA, but not FMRP, ensues. Interestingly, all FXS samples that express FMR1 full-length RNA, or after 5-AzadC-mediated transcriptional activation, the FMR1-217 mis-spliced RNA was expressed. This relationship between aberrant FMR1 expression in FXS cells and FMR1-217 was also evident in FXS iPSC-derived cells. Although the reanalysis of an RNA-seq dataset from FXS neurons with a full CGG expansion show that FMR1-217 was not produced, they did so when the FMR1 gene is specifically targeted for demethylation by CRISPR/inactive Cas9 fused to Tet1 demethylase ((18); FIG. 11C and Table 8). A second more critical point is that while FMR1-217 is generated in FXS iPSC-derived NPCs incubated with 5-AzadC, it is not produced when the CGG expansion is deleted by CRISPR/Cas9 ((17); FIG. 11C and Table 8). Therefore, the CGG expansion drives mis-spliced FMR1-217 generation.

Intellectual impairment is a major characteristic of FXS. The measurements of leukocyte full-length FMR1-205, FMR1-217, FMRP, and FMR1 gene methylation allowed correlating these molecular parameters with intelligence quotient (IQ). FMRP was moderately correlated with a higher IQ, whereas FMR1-217 was weakly correlated with a lower IQ. Based on these correlations, whether abrogating FMR1-217 RNA could elevate FMR1 and restore FMRP levels were considered. Accordingly, it was found that ASOs targeting the second exon of the FMR1-217 RNA reduced its levels in UFM FXS cells, rescued full-length FMR1 and importantly restored FMRP levels similar to TD cells. Therefore, in FXS individuals that express FMR1-217, ASO treatment can be a viable therapeutic option. In individuals with a fully methylated FMR1 locus, an ASO-based treatment would be more complex. Consider that in FXS cells with a silenced FMR1, demethylation of the locus by a chemical compound or a CRISPR/Cas9-anchored demethylating enzyme (17, 22, 34), or ASO-mediated blocking of CGG RNA translation (35, 36) have met with limited success in restoring FMRP. CRISPR/Cas9-mediated gene editing of the CGG repeats (37-40) have resulted in a nearly 70% restoration of FMRP levels. However, we show that in FXS cells with silenced FMR1, DNA demethylation combined with ASO treatment restores FMRP. Therefore, treatments that combine DNA demethylation with an ASO approach can be a useful therapeutic strategy for individuals with a fully silenced FMR1 gene.

These data demonstrate that FMR1-217 RNA is an underlying factor inhibiting FMRP expression in FMR1 RNA permissive FXS cells.

The findings suggest that ASOs can be used to correct dysregulated alternative splicing of FMR1 and restore FMRP in individuals with FXS, thereby offering a novel therapeutic strategy to treat the disorder.

### Example 11

Aberrant alternative splicing of mRNAs results in dysregulated gene expression in multiple neurological disorders. Here, it is shown that hundreds of mRNAs are incorrectly expressed and spliced in white blood cells and brain tissues of individuals with fragile X syndrome (FXS). Surprisingly, the FMR1 (Fragile X Messenger Ribonucleoprotein 1) gene is transcribed in >70% of the FXS tissues. In all FMR1-expressing FXS tissues, FMR1 RNA itself is mis-spliced in a CGG expansion-dependent manner to generate the little-known FMR1-217 RNA isoform (FMR1 isoform 12), which is comprised of FMR1 exon 1 and a pseudo-exon in intron 1. FMR1-217 is also expressed in FXS premutation carrier-derived skin fibroblasts and brain tissues. It is shown herein that in cells aberrantly expressing mis-spliced FMR1, antisense oligonucleotide (ASO) treatment reduced FMR1-217, rescued full-length FMR1 RNA, and restored FMRP (Fragile X Messenger Ribonucleoprotein) to normal levels. Notably, FMR1 gene reactivation in transcriptionally silent FXS cells using 5-aza-2-deoxycytidine (5-AzadC), which prevents DNA methylation, increased FMR1-217 RNA levels but not FMRP. ASO treatment of cells prior to 5-AzadC application rescued full-length FMR1 expression and restored FMRP. These findings indicate that misregulated RNA-processing events in blood could serve as potent biomarkers for FXS and that in those individuals expressing FMR1-217, ASO treatment offers a therapeutic approach to mitigate the disorder.

Fragile X syndrome (FXS) is a neurodevelopmental disorder causing a range of maladies including intellectual disability, speech and developmental delays, social deficits, repetitive behavior, attention deficits, and anxiety. An expansion of >200 CGG triplets in the 5′UTR of FMR1 (Fragile X Messenger Ribonucleoprotein 1) induces gene methylation and transcriptional silencing, loss of the encoded FMRP, and FXS. FMRP is an RNA-binding protein that interacts with >1,000 mRNAs in the mouse brain and human neurons, predominantly through coding region associations (Darnell et al., FMRP stalls ribosomal translocation on mRNAs linked to synaptic function and autism, Cell 146(2):247-61 (2011); Maurin et al., HITS-CLIP in various brain areas reveals new targets and new modalities of RNA binding by fragile X mental retardation protein, Nucleic Acids Res. 46(12):6344-55 (2018); Li et al., Identification of FMR1-regulated molecular networks in human neurodevelopment, Genome Res. 30(3):361-74 (2020)). FMRP generally inhibits translation and does so by stalling ribosome translocation on mRNAs (Sharma et al., Widespread Alterations in Translation Elongation in the Brain of Juvenile Fmr1 Knockout Mice, Cell Rep. 26(12):3313-22 (2019); Udagawa et al., Genetic and acute CPEB1 depletion ameliorate fragile Xpathophysiology, Nat. Med. 19(11):1473-77 (2013)), one of which encodes SETD2, which trimethylates histone H3 lysine 36 (H3K36me3) (Shah et al., FMRP Control of Ribosome Translocation Promotes Chromatin Modifications and Alternative Splicing of Neuronal Genes Linked to Autism, Cell Rep. 30(13):4459-72 (2020); Kim et al., Pre-mRNA splicing is a determinant of histone H3K36 methylation, Proc Natl Acad Sci USA. 108(33):13564-69 (2011)). Elevation of SETD2 in Fmr1-deficient mouse hippocampus results in an altered H3K36me3 chromatin landscape in gene bodies, which influences alternative pre-mRNA splicing (Shah et al., Cell Rep. 30(13):4459-72 (2020)). Many of these mis-splicing events were also detected in human postmortem autism spectrum disorder (ASD) brain and blood tissues (Gandal et al., Transcriptome-wide isoform-level dysregulation in ASD, schizophrenia, and bipolar disorder, Science 362(6240):eaat8127 (2018); Irimia et al., A highly conserved program of neuronal microexons is misregulated in autistic brains, Cell 159:1511-1523 (2014); Quesnel-Vallières et al., Misregulation of an activity-dependent splicing network as a common mechanism underlying autism spectrum disorders, Mol. Cell 64:1023-1034 (2016); Zafarullah et al., Molecular Biomarkers in Fragile X Syndrome, Brain Sci. 9(50:96 (2019); Westmark, The quest for fragile X biomarkers, Mol. Cell. Pediatr. 1(1):1 (2014)), indicating a convergence of FXS and ASD (Shah et al., Cell Rep. 30(13):4459-72 (2020); Shah et al., Do Fragile X syndrome and other intellectual disorders converge at aberrant Pre-mRNA splicing? Front. Psychiatry 12:715346 (2021)).

Leukocytes were isolated from human FXS patient tissues (blood and brain; 29 FXS males and 13 typically developing (TD) age-matched males) and RNA sequencing was performed. Analysis revealed widespread and statistically robust Misregulation of alternative splicing and RNA abundance of >1,000 mRNAs, which might provide readily available, quantifiable, and robust biomarkers for FXS.

Further analysis unexpectedly revealed that FMR1 RNA was expressed in 21 of 29 FXS leukocyte samples, some nearly as high as FMR1 transcript levels from TD individuals. This was a surprising result because the FMR1 locus harboring>200 CGG repeats and full methylation is purported to be silent. However, the highest FMR1 RNA-expressing FXS individuals were mosaic (CGG repeat number mosaicism or partial methylation of a full expansion). Furthermore, it was found that much of the FMR1 mRNA in the FXS samples was itself mis-spliced to generate FMR1-217 (ENST00000621447.1), a 1.8 kb isoform comprised of FMR1 exon 1 and a pseudo-exon within FMR1 intron 1. This isoform could encode a truncated 31 amino acid polypeptide whose function, if any, is unknown. Additional analysis revealed that FMR1-217 was detected in FXS dermal and lung-derived fibroblasts as well as in FXS postmortem cortex samples, indicating the preponderance of FMR1 mis-splicing in FXS populations. Fibroblasts from some FXS premutation (i.e., about 55 to 200 CGG repeats) male carriers also express FMR1-217 RNA, indicating that mis-splicing may be widespread in other disorders linked to CGG expansions in FMR1.

These findings raised the intriguing possibility that modulation of FMR1-217 mis-splicing might result in increased FMRP levels. Accordingly, eleven 2′-O-methoxy ethyl (MOE)/phosphorothioate-containing antisense oligonucleotides (ASOs) were generated against several regions of FMR1-217 and transfected into FXS lymphoblast cells expressing this transcript. A combination of two ASOs blocked improper FMR1 splicing, rescued proper FMR1 splicing, and restored FMRP to TD levels. Moreover, application of the DNA methylation inhibitor 5-aza-2′-deoxycytidine (5-AzadC) to a second FXS lymphoblast line as well as FXS fibroblast lines that normally do not express any FMR1 resulted in the synthesis of both FMR1 and FMR1-217 RNAs but little FMRP. However, treatment of these cells with both 5-AzadC and the ASOs produced strong FMRP upregulation. These studies demonstrate that first, in cells from FXS but not TD individuals, a significant proportion of the FMR1 RNA is mis-spliced to produce the FMR1-217 isoform. Second, ASO treatment to reduce FMR1-217 levels resulted in FMRP restoration to TD levels. This study provides a basis for further optimizing strategies to reduce the mis-splicing of FMR1 RNA and offers a unique therapeutic approach to mitigate FXS using splice-switching ASOs.

### Results

Gene Expression Changes in Leukocytes from FXS Individuals.

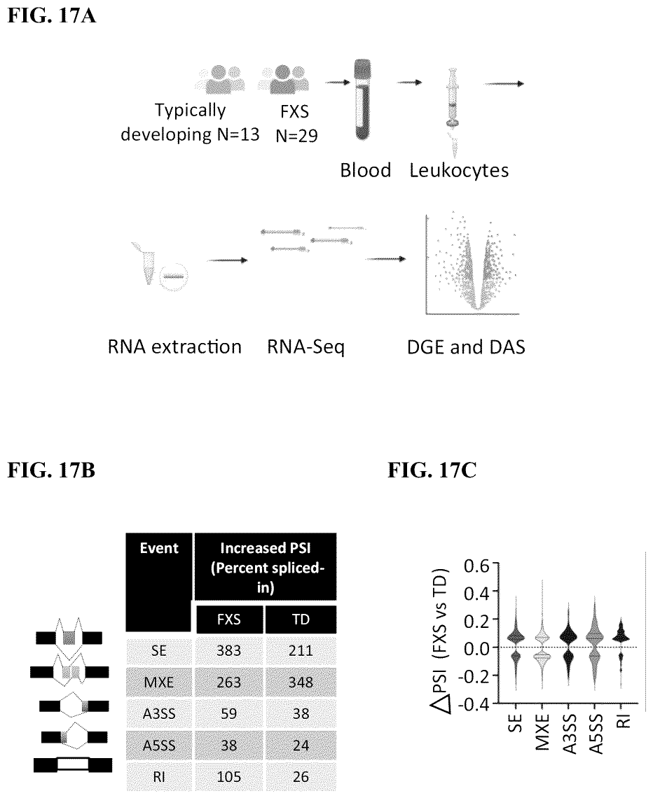

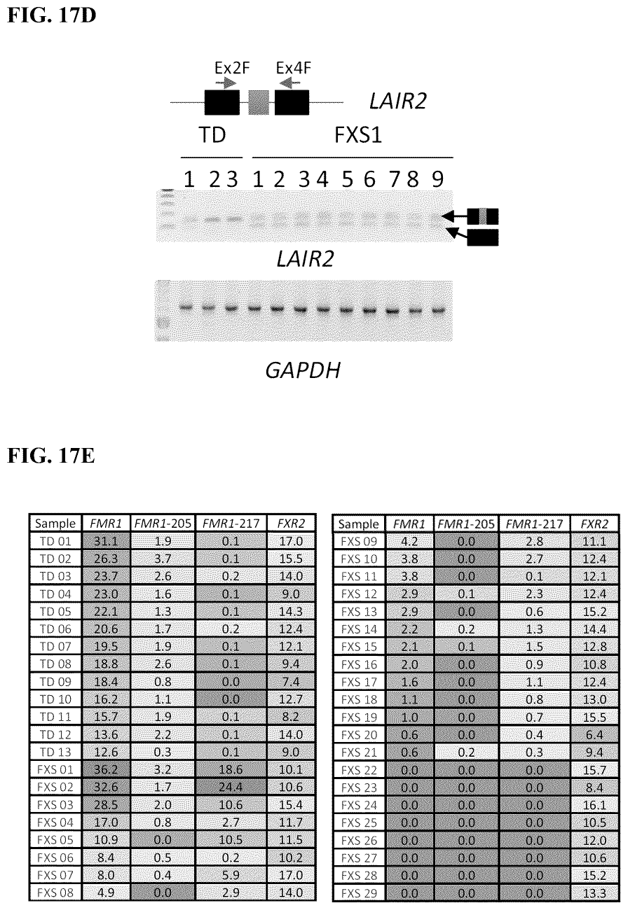

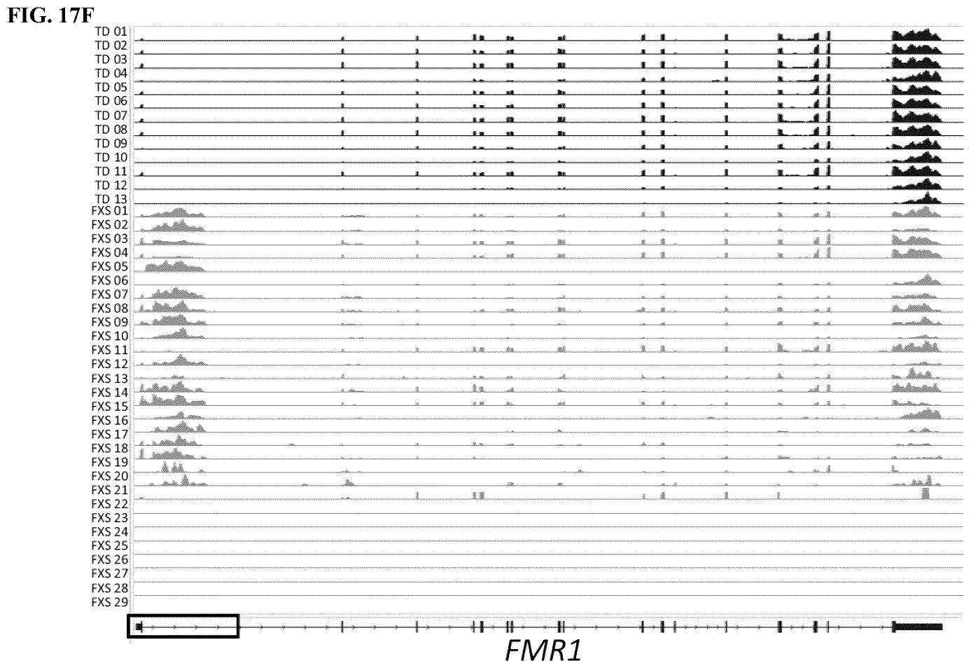

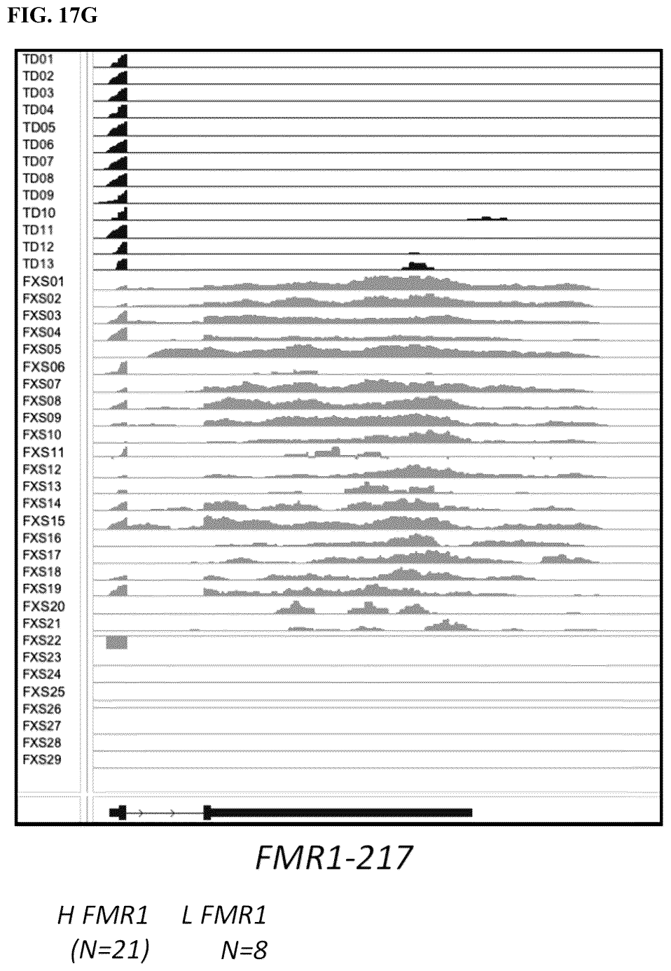

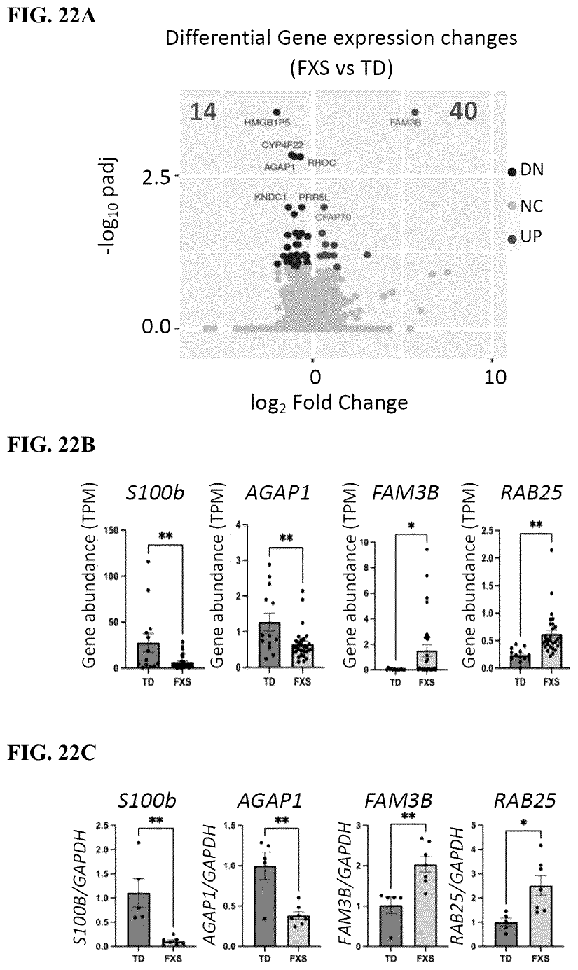

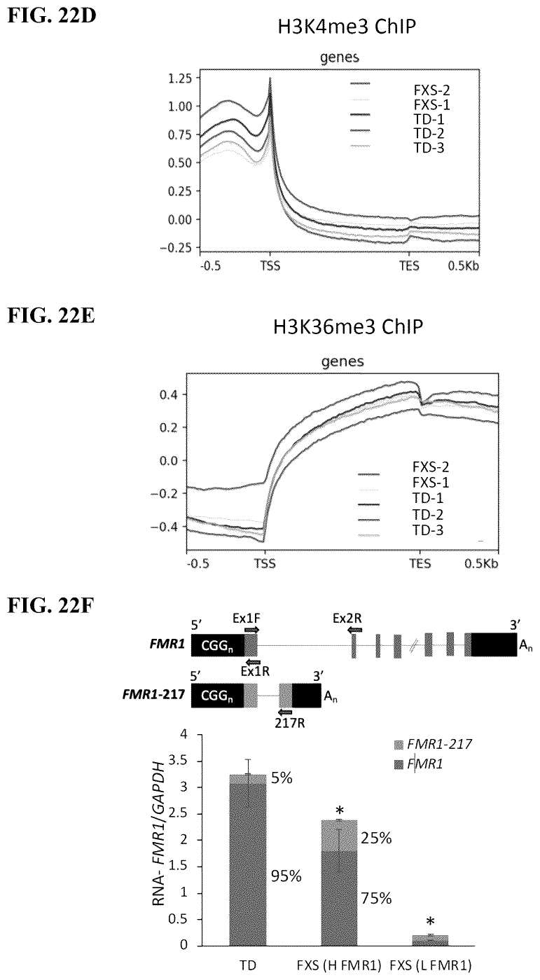

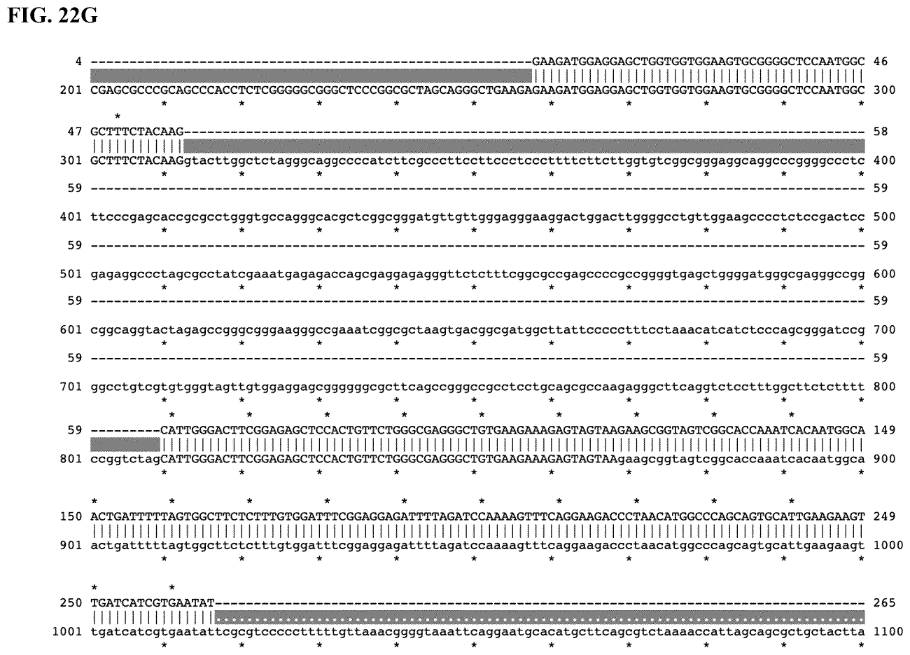

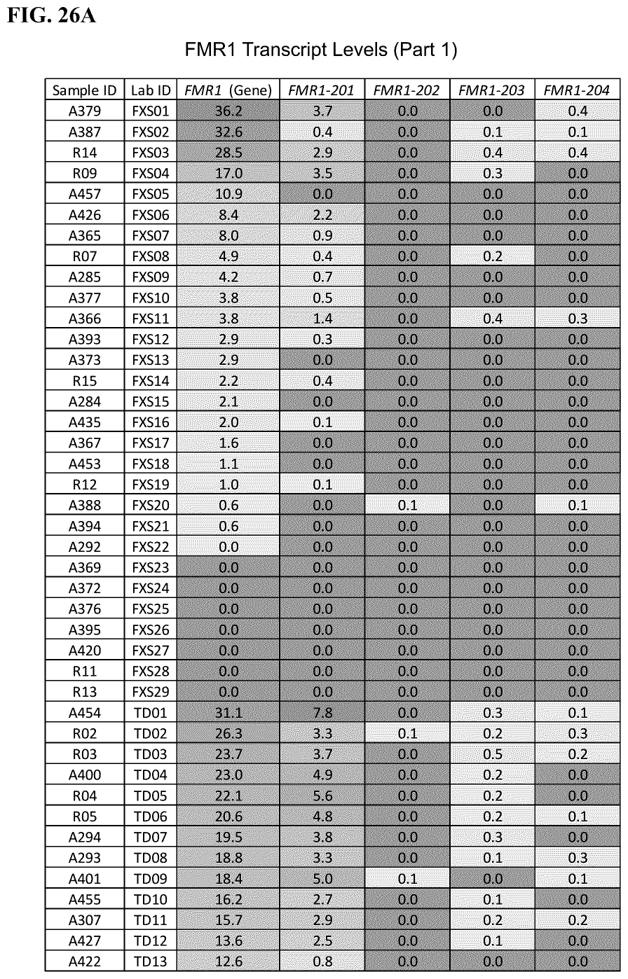

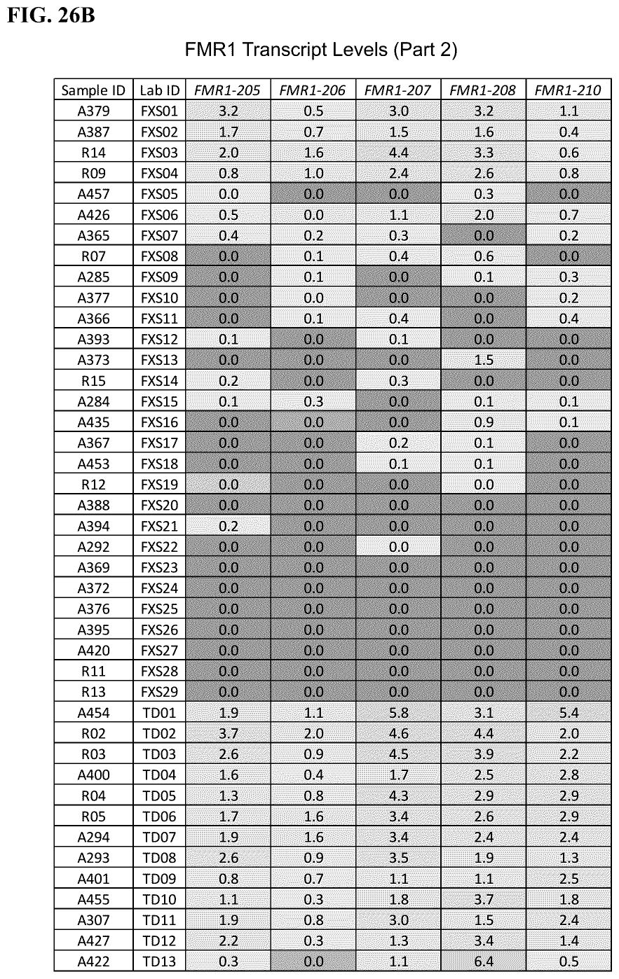

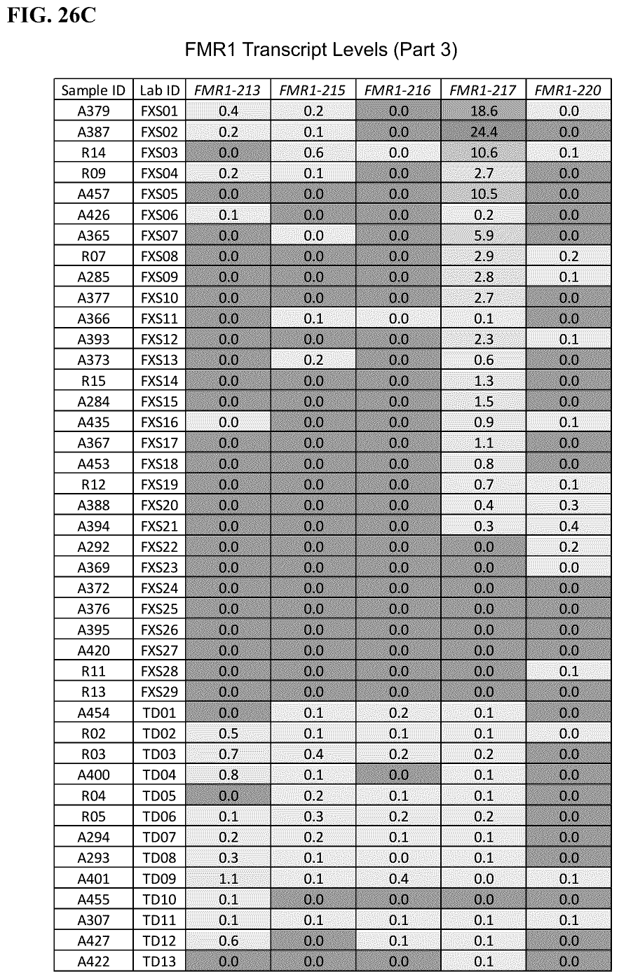

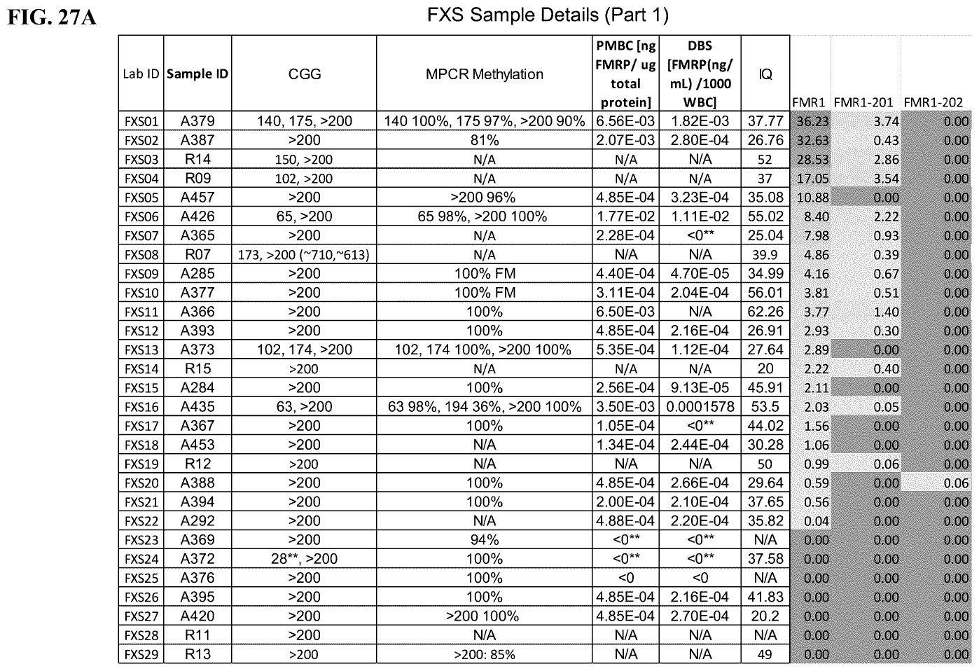

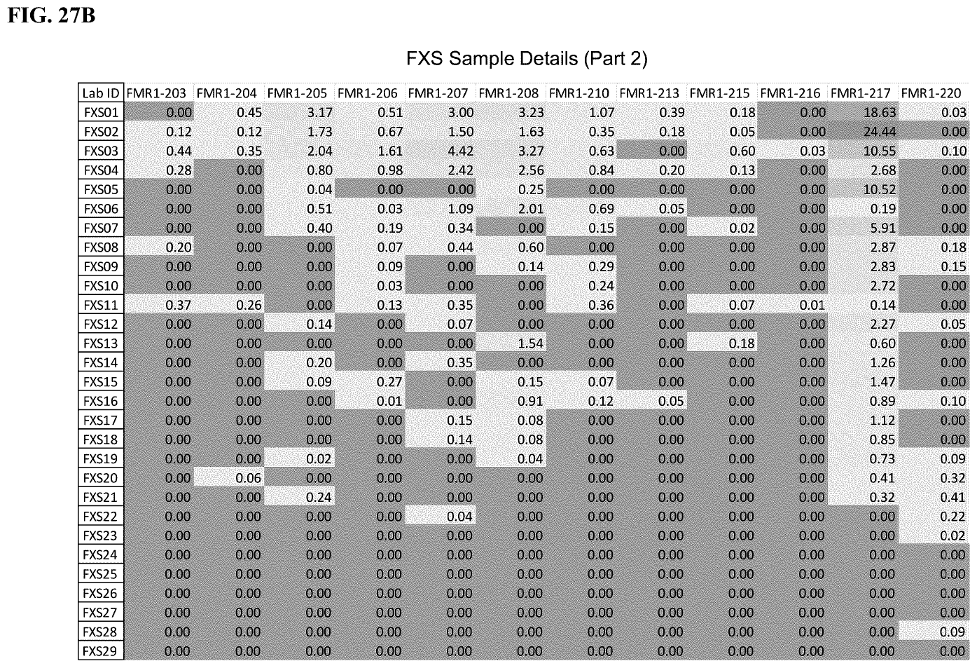

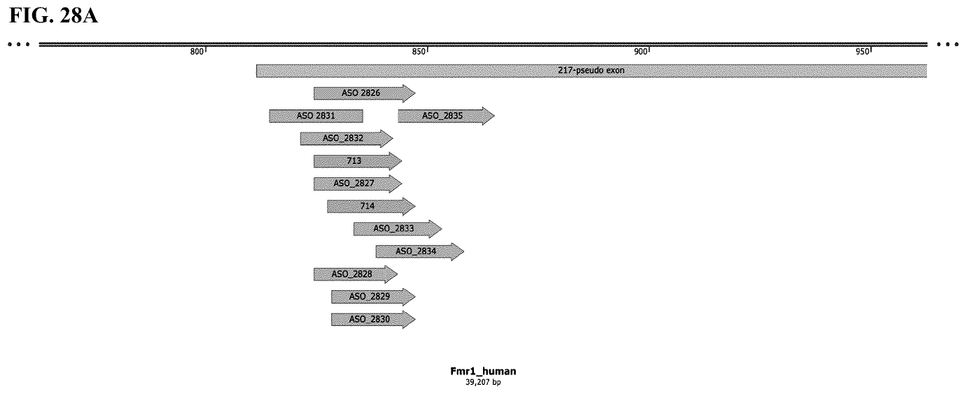

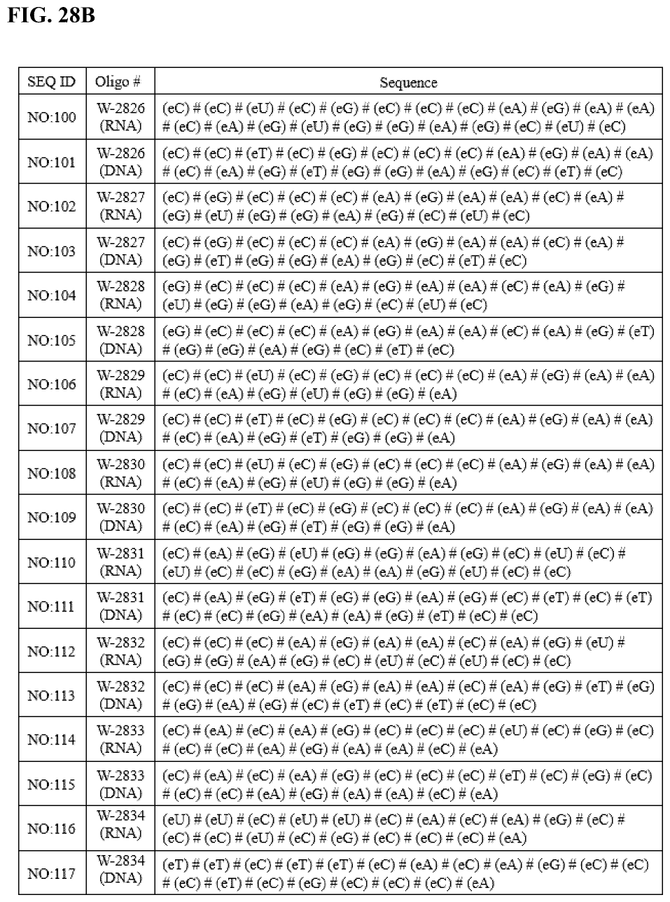

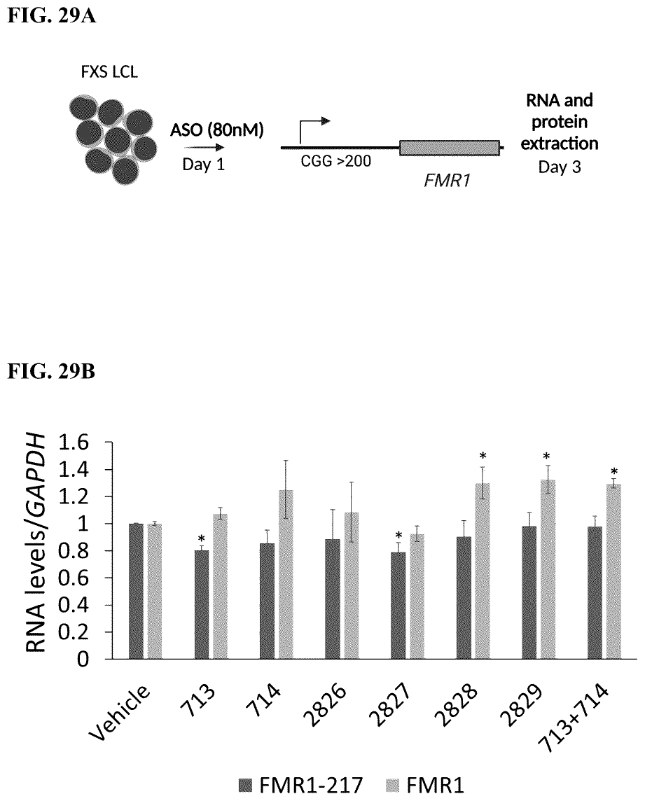

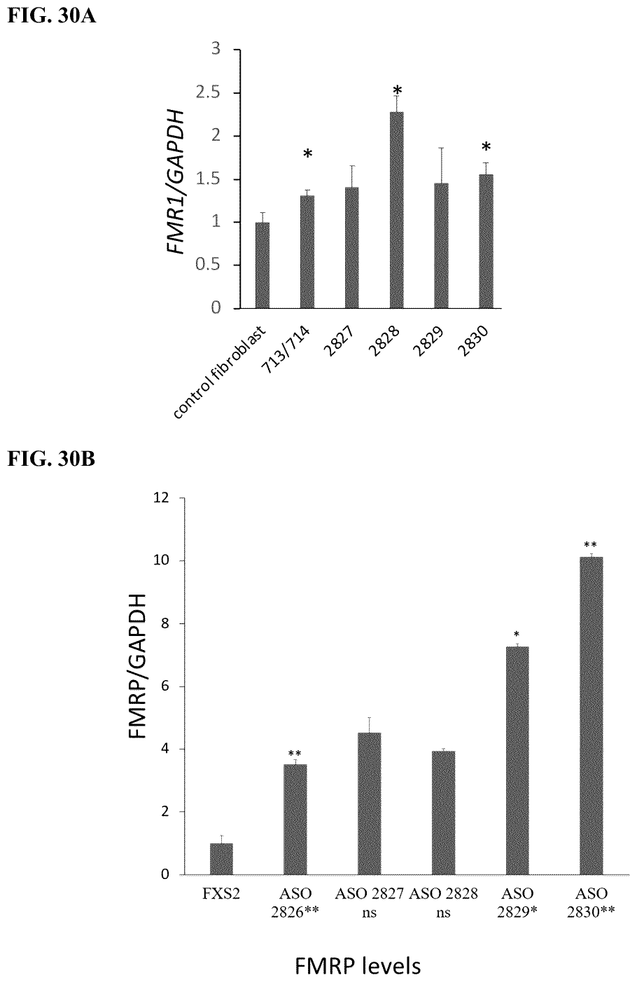

Aberrant splicing of mRNAs is evident in the hippocampal tissue from Fmr1 KO mice, many of which overlap with those in human postmortem autistic cortex (Shah et al., FMRP Control of Ribosome Translocation Promotes Chromatin Modifications and Alternative Splicing of Neuronal Genes Linked to Autism, Cell Rep. 30(13):4459-72 (2020); reviewed in Shah et al., Do Fragile X Syndrome and Other Intellectual Disorders Converge at Aberrant Pre-mRNA Splicing? Front. Psychiatry 12:715346 (2021); and Richter et al., The molecular biology of FMRP: New insights into fragile X syndrome, Nat. Rev. Neurosci. 22:209-222 (2021)). To investigate whether mis-splicing of mRNAs also occurs in blood samples from FXS individuals, deep (60 to 90 million reads) and long-read (150PE) RNA-seq was performed on freshly obtained leukocytes from 29 FXS males and 13 age-matched typically developing (TD) males (FIG. 17A). CGG repeat expansion (>200) for all samples and FMR1 promoter methylation status for FXS samples when available were confirmed by either southern blot or methylation PCR assays (Tables 9-12 and FIGS. 26A-27B). Differential alternative splicing (DAS) (Shen et al., rMATS: Robust and flexible detection of differential alternative splicing from replicate RNA-Seq data, Proc. Natl. Acad. Sci. U.S.A. 111:E5593-E5601 (2014)) analysis revealed hundreds of statistically significant events that were altered between genotypes (FXS vs. TD) (using an FDR<5% and a difference in the exon inclusion levels (PSI, Percent spliced-in) of >5% (FIG. 17B and Tables 13-19). A violin plot of the DAS demonstrates that most significant splicing changes were about ±10% in FXS vs. TD with some changes near 30 to 40% (FIG. 17C). RT-PCR was used to confirm that the increased skipping (˜20% in DAS analysis) of exon 3 in the LAIR2 (leukocyte-associated immunoglobulin-like receptor 2) RNA (FIG. 17D and Tables 13-19). Differential gene expression (DGE) was assessed, and it was found that about 50 RNAs were up- or down-regulated in FXS leukocytes relative to TD (P value<0.0002) (FIG. 22A and Tables 13-19) and were clustered based on their z-scores (Tables 13-19). S100B (S100 calcium-binding protein B), AGAP1 (ArfGAP With GTPase Domain, Ankyrin Repeat And PH Domain 1), FAM3B (FAM3 Metabolism-Regulating Signaling Molecule B), and RAB25 (RAS oncogene family member 25) are examples of RNAs that were altered in the FXS samples relative to TD (log 2FC, P value<0.0002) (FIG. 22B) and confirmed by RT-qPCR (FIG. 22C).

Fmr1-dependent changes in the epigenetic mark H3K36me3 correlate with aberrant alternative splicing in the mouse hippocampus (Shah et al., FMRP Control of Ribosome Translocation Promotes Chromatin Modifications and Alternative Splicing of Neuronal Genes Linked to Autism, Cell Rep. 30(13):4459-72 (2020)). Fmr1-dependent changes in RNA levels were also correlated with H3K4me3 in cultured mouse neurons (Korb et al., Excess translation of epigenetic regulators contributes to Fragile X syndrome and is alleviated by Brd4 inhibition, Cell 170:1209-1223 (2017)). ChIP-Seq was performed to determine whether similar changes in chromatin marks occur in FXS cells. However, results from FXS (n=2) and TD (n=3) leukocyte samples showed no genotype-specific changes in H3K36me3 or H3K4me3 (FIG. 22D and FIG. 22E). In summary, hundreds of statistically significant events that distinguish between TD and FXS in leukocytes represent robust, unique, and easily obtained biomarkers for human FXS.

### FMR1 RNA is Expressed and Mis-Spliced in a Subset of FXS Individuals.

Expansion of >200 CGG repeats in FMR1 induces gene methylation, transcriptional silencing, loss of FMRP, and FXS. It was therefore surprising that in 21 of 29 FXS individuals, FMR1 RNA isoforms were detected, and in four individuals, the levels were similar to or even higher than those in the TD individuals (FIG. 17E, FMR1 RNA TPM levels). When only full-length FMR1-encoding 632 amino acid FMRP (FMR1-205) was examined, white blood cells (WBCs) from six individuals had levels similar to those of TD (FIG. 17E). For comparison, the levels of the FMR1 paralog FXR2 were unchanged (FIG. 17E). FMR1 RNA reads were detected at robust levels in TD individuals (blue traces), and also in FXS individuals (coral traces) (FIG. 17F). FXS individuals 1-18 expressed relatively high FMR1 levels (with a cutoff of 0.6 TPM) (H FMR1) compared to FXS individuals 19-29 who expressed low or undetectable FMR1 levels (L FMR1) (FIG. 17E and FIG. 17F). Remarkably, the H-FMR1 FXS individuals displayed RNA reads in intron 1 of FMR1 (black box, enlarged in the panel below, FIG. 17G) that were notably absent in TD individuals even though FMR1 RNA was strongly expressed (FIG. 17G). The FMR1 RNA reads detected in FMR1 intron 1 correspond to the second exon of the FMR1-217 RNA isoform. FMR1-217 (ENST00000621447.1) is a 1.8 kb transcript comprised of two exons and is predicted to encode a 31 amino acid polypeptide. Notably, most of the total FMR1 RNA in the FXS samples comprised of the aberrantly spliced FMR1-217 transcript, which was absent in samples from TD individuals (FIG. 17G, Tables 9-12, and FIGS. 26A-27B). TPMs of all 14 FMR1 isoforms detected in the TD and FXS patient samples are shown in Tables 9-12 and FIGS. 26A-27B.

Next, the proportion of full-length FMR1 RNA to FMR1-217 RNA was assessed. In the TD samples, 95% of the total FMR1 RNA (primers Ex1F and Ex1R) represents full-length molecules (primers Ex1F and Ex2R), whereas in the H FMR1 samples, 75% of the total FMR1 RNA was full length and 25% was FMR1-217 (primers Ex1F and 217R) (FIG. 22F). In the L FMR1 samples, both isoforms were barely detected. The total FMR1 RNA levels were normalized to GAPDH RNA (*P values<0.05). The presence of FMR1-217 RNA in FXS leukocytes was confirmed by sequencing the amplified product generated using RT-qPCR (reverse transcription primed with oligodT) (FIG. 22G). Importantly, all FXS individuals in this study irrespective of FMR1 expression displayed typical FXS symptoms which may be due to the negligible amounts of FMRP even in patients with high FMR1 expression (Tables 9-12 and FIGS. 26A-27B).

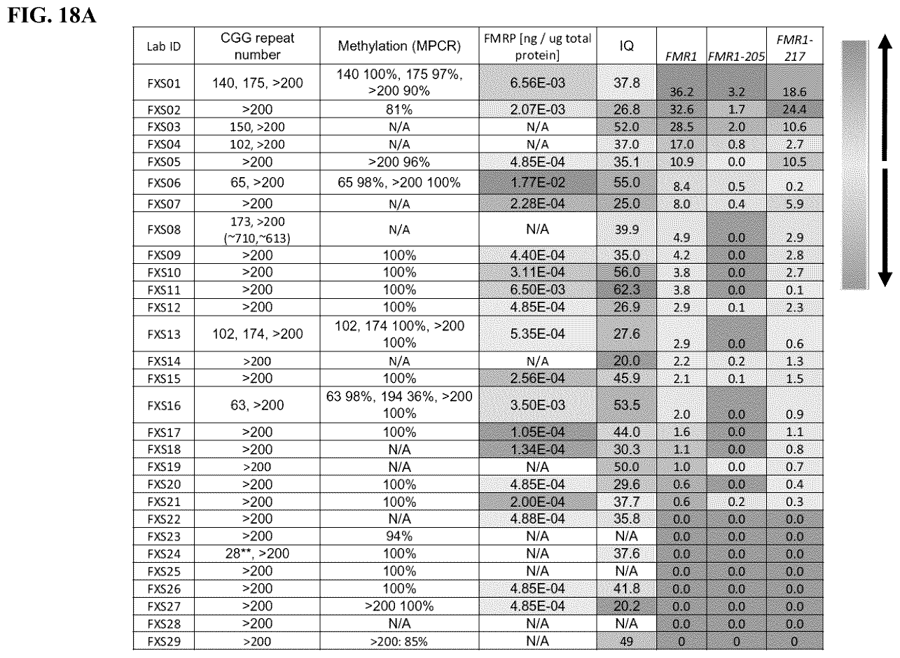

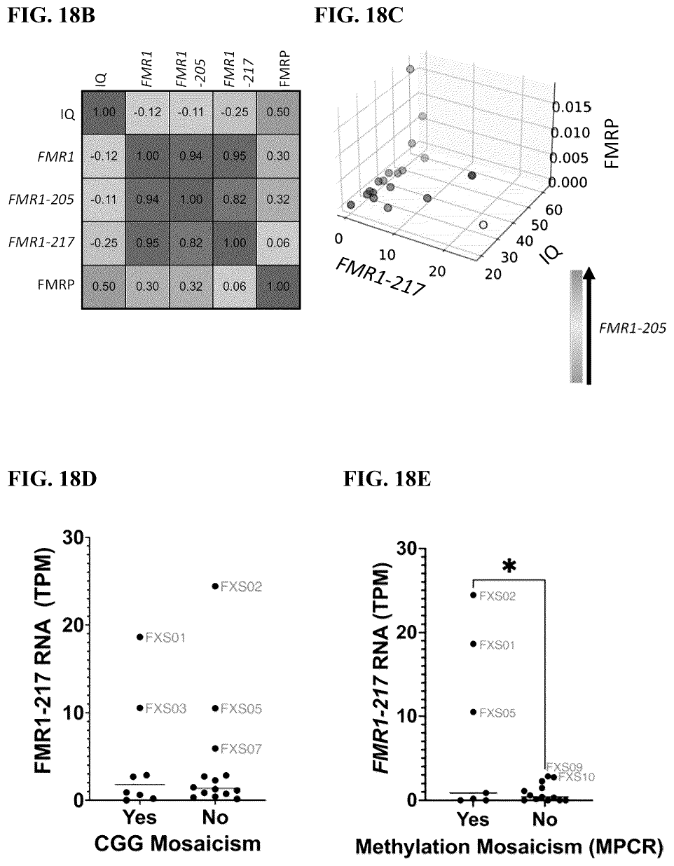

Whether the parameters measured in WBCs correlated with intelligence quotient (IQ) was investigated. FIGS. 18A-18B and 26A-27B and Tables 9-12 present determinations of methylation status of the FMR1 gene (by PCR), FMRP levels (ng/μg total protein), CGG repeat number, FMR1-217, full-length FMR1-205 (in TPM) and all detected FMR1 isoforms (TPM), and IQ (Stanford-Binet test).

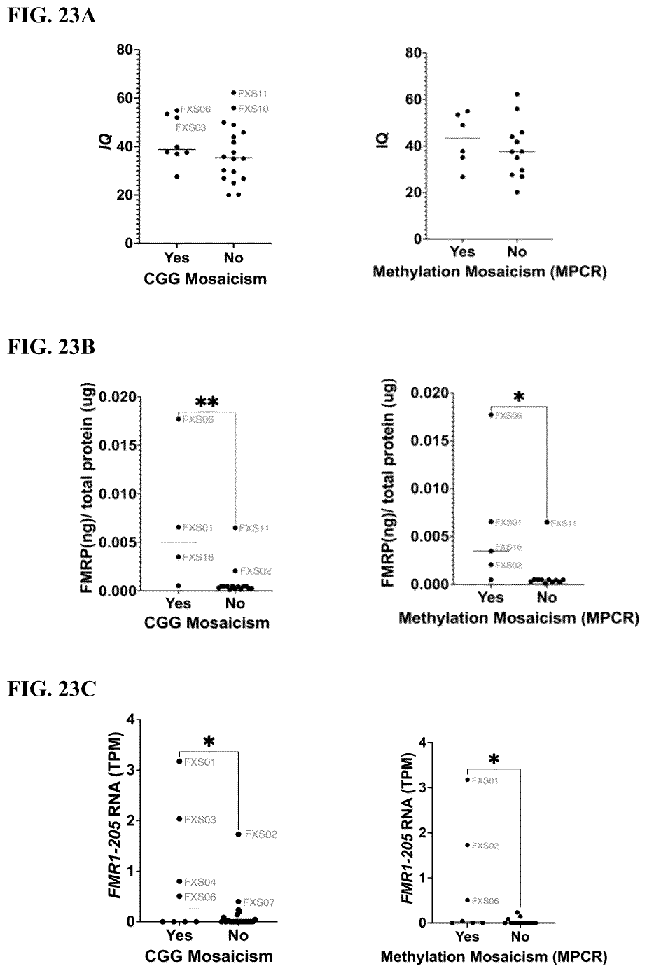

Total FMR1 RNA expression was highly correlated to FMR1-217 and FMR1-205 isoform expression (FIG. 18B). More intriguing is the moderately positive correlation of IQ with FMRP protein levels. Surprisingly, FMR1-205, which encodes full-length FMRP, had no correlation with IQ. FMR1-217 has a negative correlation with IQ, perhaps indicating a deleterious effect of this isoform. FIG. 18C displays a 3-dimensional comparison of all the parameters noted above. The FXS patients toward the upper end of the IQ range (20 to 60) showed lower FMR1-217 levels and higher FMRP levels. Next, the influence of mosaicism (CGG repeat number or methylation) on FMR1-217 expression in the FXS patients was assessed (FIG. 18A and FIG. 18D). Although 38% of the FXS patients (8 of 21) had alleles for both the premutation (55 to 200 CGG repeats) and the full CGG expansion (>200 CGG repeats), the CGG mosaicism did not significantly influence the FMR1-217 expression (FIG. 18D). The IQ of the patients with or without CGG repeat mosaicism was also comparable (FIG. 23A). However, FMRP levels and FMR1-205 expression were significantly reduced in patients with no CGG mosaicism (FIGS. 23B-23C), suggesting a reduced capacity of the full expansion alleles to produce FMRP but not the mis-spliced FMR1-217 RNA. The methylation mosaicism was a strong determinant of FMR1-217 (FIGS. 18A and 18E), FMRP (FIG. 23B), and FMR1-205 (FIG. 23C) expression but not IQ (FIG. 23A). These results show several important findings. First, the FMR1 locus in FXS is frequently transcribed to produce the mis-spliced FMR1-217 RNA irrespective of CGG mosaicism and in a few cases when the locus is fully methylated. Second, FMRP levels in WBCs are positively correlated with IQ. Third, the negative correlation of FMR1-217 with IQ suggests that the process of mis-splicing, the 31-amino acid polypeptide derived from FMR1-217, or the FMR1-217 RNA itself (or all three), might impart some toxic effect manifest in the brain (e.g., IQ). In any event, the levels of FMR1-217 expression as well as hundreds of additional transcriptome-wide changes in RNA processing events may form the basis for molecular stratification of FXS individuals.

### FMR1-217 Is Expressed in Human FXS and Premutation Carrier Postmortem Brain.

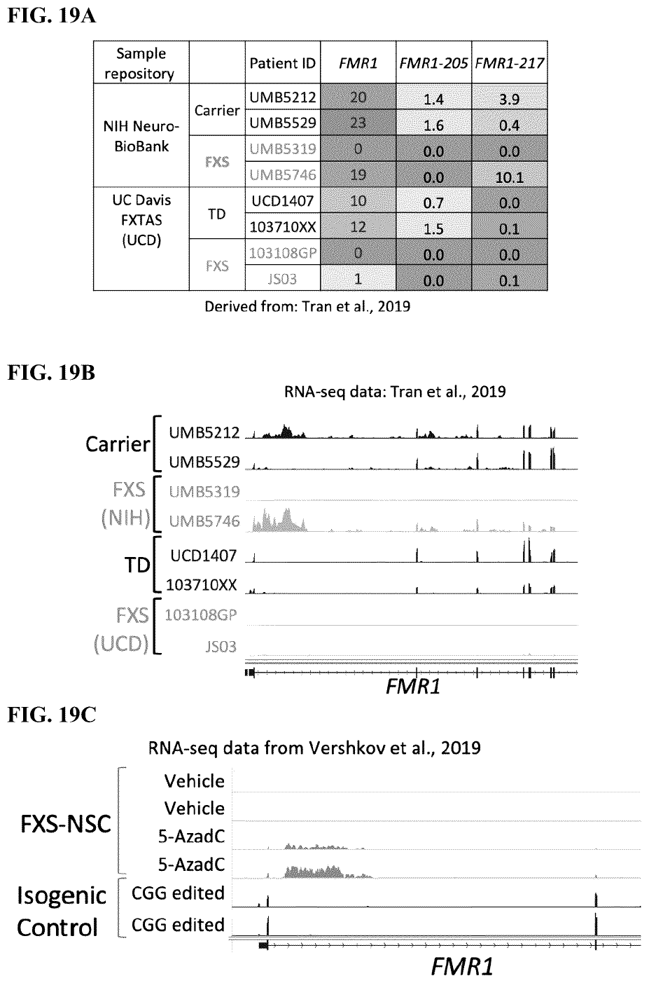

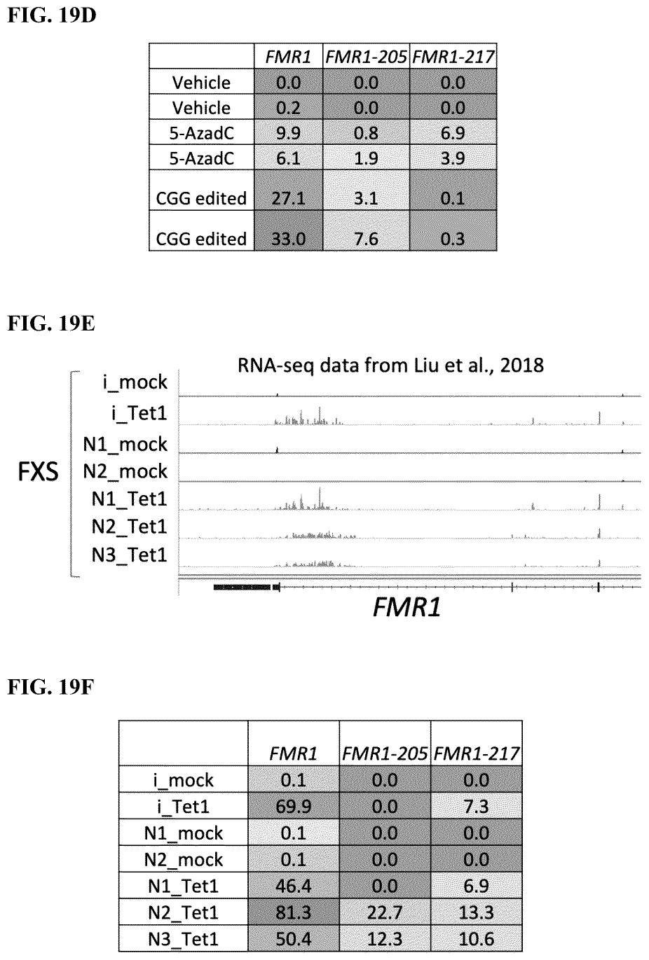

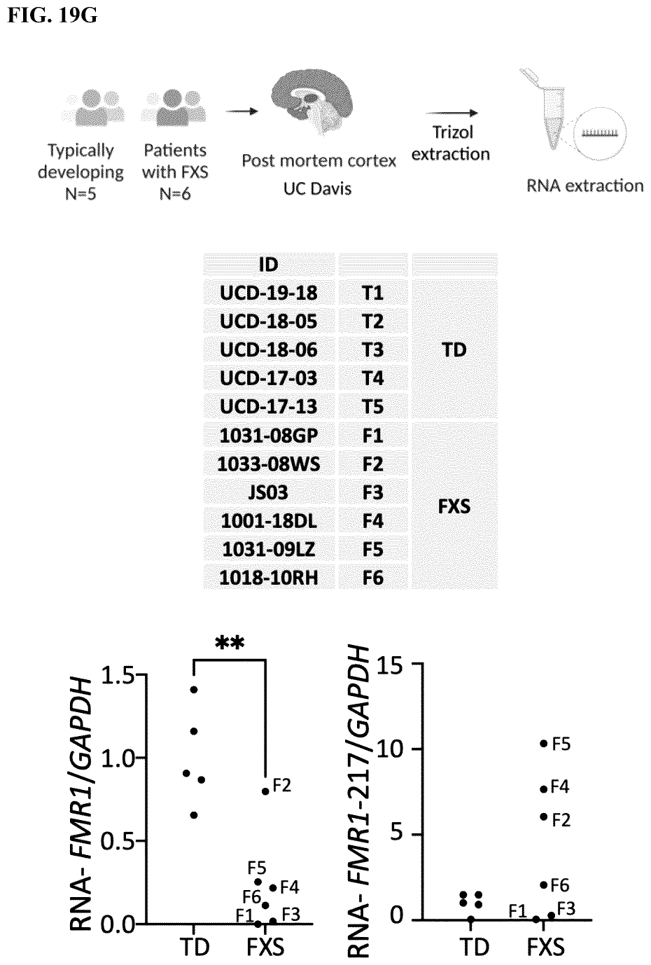

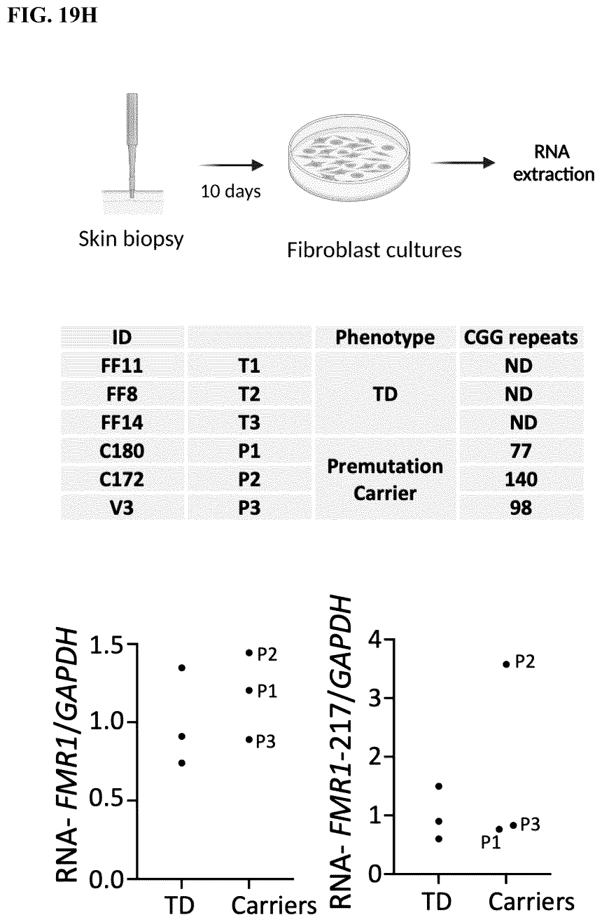

To investigate whether FMR1-217 is expressed in FXS brain, publicly available RNA-seq data of postmortem frontal cortex tissues from FXS individuals, premutation carriers (CGG repeats 55-200), and TD individuals (Tran et al., Widespread RNA editing dysregulation in brains from autistic individuals, Nat. Neurosci. 22:25-36 (2019)) were analyzed. FMR1 RNA (TPM) levels were highest in premutation carriers (FIG. 19A). Interestingly, the FXS sample UMB5746, which displays CGG mosaicism, displayed high levels of FMR1 RNA (FIGS. 19A-19B) and to a lesser extent, FMRP (Tran et al., Nat. Neurosci. 22:25-36 (2019)). The analysis showed that this individual expressed FMR1-217 as did FXS carrier UMB5212, who had fragile X-associated tremor/ataxia syndrome (FXTAS) (FIGS. 19A-19B). Neither TD individual expressed FMR1-217 (FIGS. 19A-19B). Thus, FMR1-217 RNA may be expressed in the brains of a subset of FXS individuals and premutation carriers.

It is found that in FXS individuals with a transcriptionally active FMR1 gene, FMR1-217 was expressed. Next, it was investigated whether demethylating the locus in a transcriptionally silent FXS cell line can result in FMR1-217 expression and whether FMR1 mis-splicing is CGG repeat expansion dependent. RNA-seq data from Vershkov et al. (FMR1 reactivating treatments in Fragile X iPSC-derived neural progenitors in vitro and in vivo, Cell Rep. 26:2531-2539 (2019)), where the FMR1 locus was reactivated in transcriptionally silent FXS iPSC-derived neural stem cells (NSCs) by either treatment with 5-AzadC, a nucleoside analog that prevents DNA methylation, or by CRISPR/Cas9 editing to delete the CGG expansion from FMR1 locus, were reanalyzed. Reanalysis of these data (FIG. 19C) and FMR1 transcript quantification (TPM) in FIG. 19D showed reads corresponding to FMR1-217 (in coral) in the FXS-NSCs upon incubation with 5-AzadC. Moreover, loss of the CGG repeats in the edited cells resulted in no FMR1-217 reads but instead robust expression of full-length FMR1. Total FMR1 and FMR1-205 were expressed in the CGG-edited and 5-AzadC-treated cells but not in vehicle-treated cells. This analysis demonstrated that FMR1-217 is indeed derived from the FMR1 locus and requires a CGG expansion.

In a complementary study, Liu et al. (Rescue of Fragile X syndrome neurons by DNA methylation editing of the FMR1 gene, Cell 172:979-991 (2018)) performed a targeted FMR1 gene demethylation experiment by incubating FXS iPSC and FXS iPSC-derived neurons with an FMR1 small-guide RNA and a catalytically inactive Cas9 fused to Tet1 demethylase sequences. Reanalysis of the Liu et al. (Cell 172:979-991 (2018)) subsequent RNA-seq data is shown in (NaN) MR1 transcript quantification (TPM) in FIG. 19F. FMR1-217 reads were evident only when the gene was demethylated in the FXS cells. Total FMR1 and FMR1-205 expression was detected in the Tet1-treated samples (inexplicably, no FMR1-205 in sample N1_Tet1). These data therefore show once again that FMR1-217 is derived from the FMR1 locus in iPSCs and iPSC-derived neurons and requires a CGG expansion.

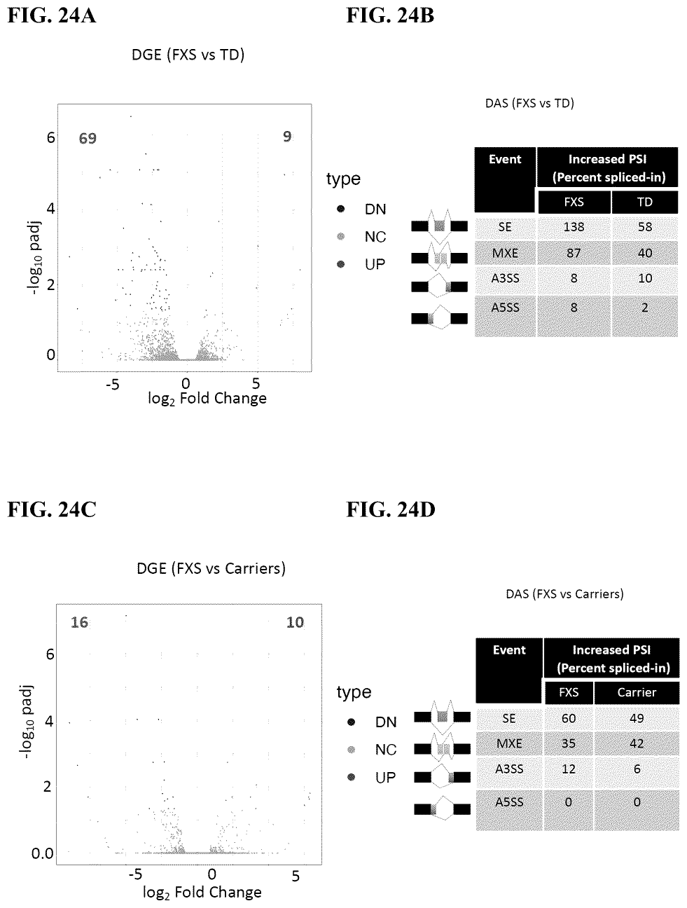

To determine whether transcriptome-wide changes in RNA expression could be detected in the frontal cortex, DGE and DAS analysis was performed on RNA-seq data (Tran et al., Widespread RNA editing dysregulation in brains from autistic individuals, Nat. Neurosci. 22(1):25-36 (2019)) from the FXS vs. TD (FIG. 24 and Tables 20-28). Statistically significant changes were observed in DGE (FIG. 24A and Tables 20-28) and DAS events (FIG. 24B and Tables 20-28). A comparison of FXS samples to FXS premutation carrier samples also showed significant DGE (FIG. 24C and Tables 20-28) and DAS events (FIG. 24D and Tables 20-28). Thus, hundreds of transcriptomic changes are identified in brain tissues from FXS individuals and FXS carriers.

To confirm the expression of FMR1-217 RNA in FXS brain tissue, frozen postmortem cortex samples from six FXS males and five age-matched TD males (UC Davis Health) were obtained. Using RT-qPCR, it was found that the FMR1 full-length RNA was significantly reduced in the FXS individuals compared to that in the TD individuals. However, three of the six FXS individuals expressed varying levels of the FMR1 full-length RNA as well as FMR1-217 RNA (1031-09LZ, 1001-18DL, and 1033-08WS) (FIG. 19G). Previous studies on the FXS sample 1031-09LZ have noted expression of FMR1 RNA similar to that in TD individuals, despite the presence of a methylated fully mutated FMR1 locus (Esanov et al., The FMR1 promoter is selectively hydroxymethylated in primary neurons of fragile X syndrome patients, Hum. Mol. Genet. 25:4870-4880 (2016)). However, no detectable FMRP was found in the FXS brain sample 1031-09LZ (Raj et al., Cell-type-specific profiling of human cellular models of fragile X syndrome reveal PI3K-dependent defects in translation and neurogenesis, Cell Rep. 35:108991 (2021)). In agreement with observations here, RNA-seq data from Tran et al. showed no FMR1 RNA in the FXS tissue samples (1031-08GP and JS03) (FIG. 19I and FIG. 19B) as well as an absence of FMRP (Tran et al., Widespread RNA editing dysregulation in brains from autistic individuals, Nat. Neurosci. 22(1):25-36 (2019)).

To gain greater insight into the relationship of FMR1-217 FXS carrier tissue (CGG repeats between 55 and 200), skin biopsies were obtained from three additional premutation carriers and three TD individuals (FIG. 19H). The skin samples were cultured in vitro to generate fibroblast cell lines for RNA analysis. Interestingly, FMR1-217 was detected, using RT-qPCR, in one premutation carrier (C172) with 140 CGG repeats but not in samples with 77 or 98 CGG repeats (FIG. 19H). There was no change in total FMR1 RNA levels among the samples (FIG. 19H). Thus, generation of FMR1-217 may be linked to the number of CGG repeats in the FMR1 gene.

ASOs Targeting FMR1-217 Restore FMRP Levels in FXS Cell Lines with Partial or Complete FMR1 Gene Methylation.

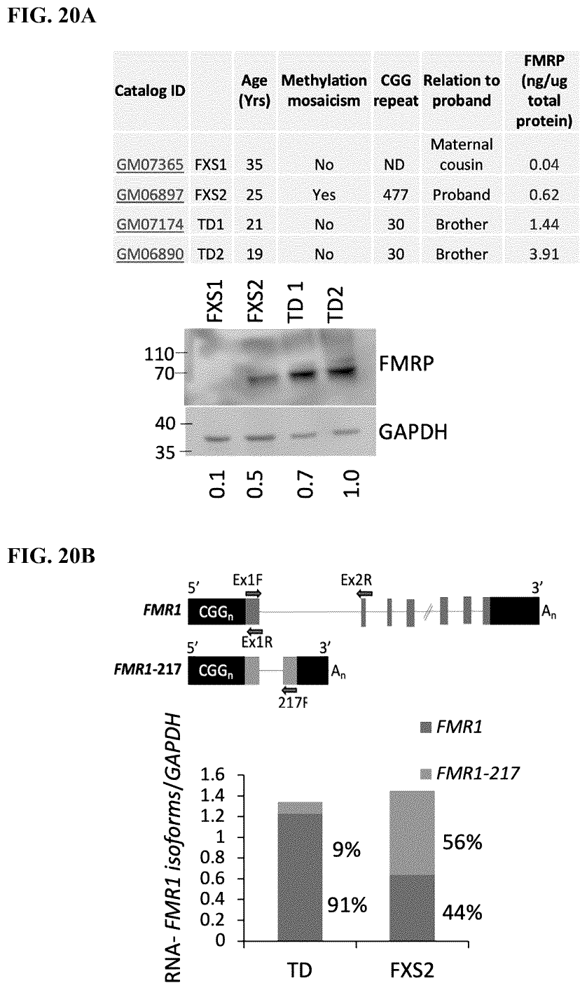

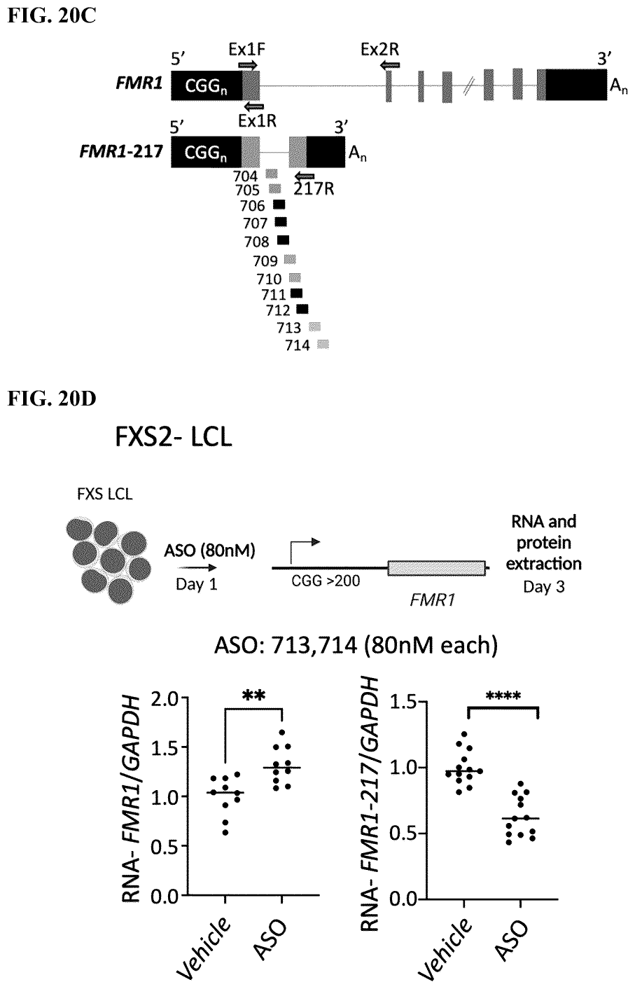

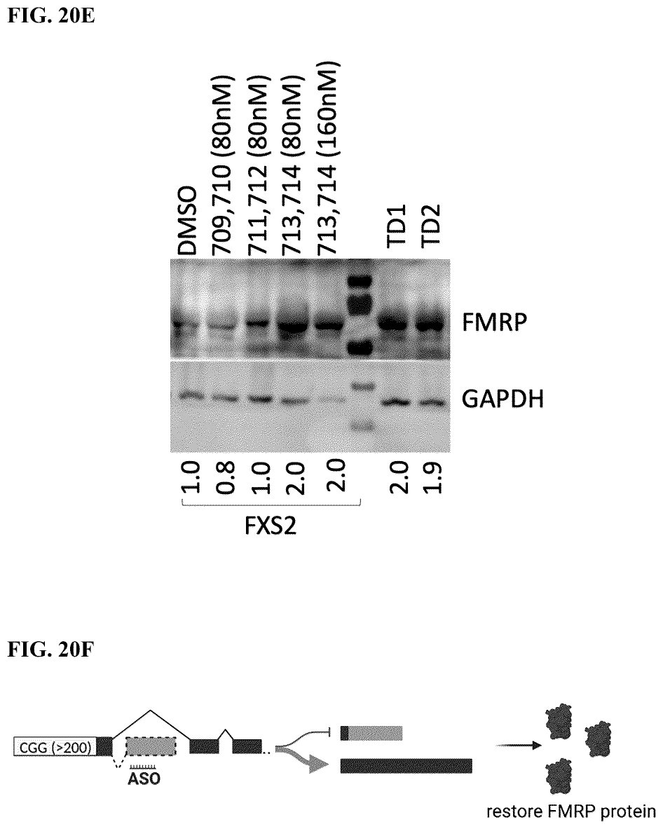

Next, whether blocking the formation of FMR1-217 could lead to an increase in full-length FMR1 and concomitantly an increase in FMRP, was investigated. For these experiments, lymphoblast cell lines (LCLs), derived from an FXS individual with a fully methylated locus that is transcriptionally inactive (FXS1, GM07365), an FXS individual with a partially methylated locus that expresses FMR1 RNA (FXS2, GM06897), and two TD individuals (TD1, GM07174, and TD2, GM06890) (all samples from Coriell Institute, NJ), were used (FIG. 20A). Western blot analysis showed that modest levels of FMRP were detected in FXS2, but not FXS1 cell lines. FMRP was strongly expressed in TD1 and TD2 cells (ratios of FMRP/GAPDH relative to TD2 are shown below the blot) (FIG. 20A). Similar ratios of FMRP protein in these cell lines were obtained by the LUMINEX® Microplex immunochemistry assay (FMRP levels in ng FMRP/μg total protein) (FIG. 20A). Using RT-qPCR, FMR1-217 RNA was found to be expressed in FXS2 LCLs and comprise 56% of the total FMR1 RNA compared to only 9% in TD cells (FIG. 20B). It is noteworthy that although total FMR1 RNA levels in FXS2 cells were similar to those in TD cells, FMRP levels were much lower (FIGS. 20A-20B).

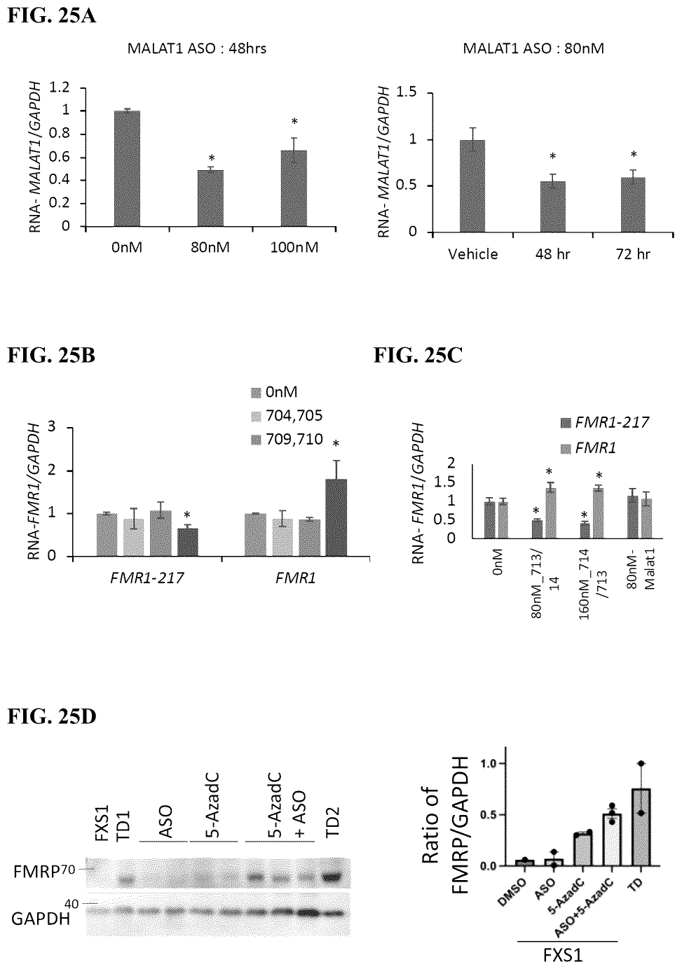

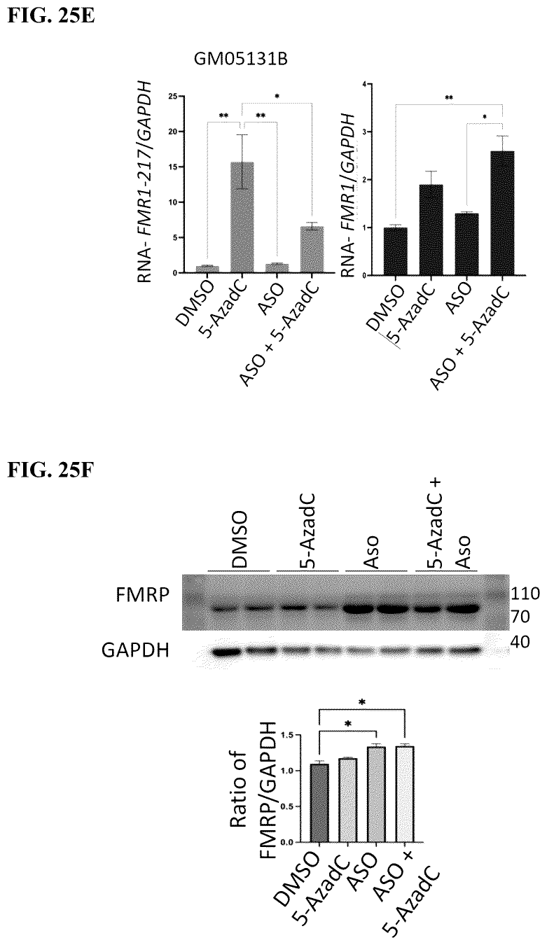

Next, eleven antisense oligonucleotides (ASOs) modified with 2′-O-methoxyethyl-RNA (MOE) at each nucleotide were generated. The ASOs were tiled across intron 1, the intron 1 and the pseudo-exon junction, or within the pseudo-exon of FMR1-217 RNA (FIG. 20C), according to standard guidelines for design of exon-skipping ASOs. To test transfection efficiency under the conditions of our experiment, a gapmer ASO (80 nM) targeting MALAT1 RNA (Moazami et al., Quantifying and mitigating motor phenotypes induced by antisense oligonucleotides in the central nervous system, bioRxiv 2021.02.14.431096 (2021)) was used for 72 hours, which led to about a 60% decrease in MALAT1 RNA levels (FIG. 25A). Among the ASOs tested in FXS2 (FIG. 25B), the combination of ASO 713 and 714 (80 nM each) led to a significant decrease in FMR1-217 and an increase in full-length FMR1 (FIGS. 20D and 25C). ASOs 713 and 714 at 80 nM or 160 nM each for 72 hours elicited similar decreases in FMR1-217 and increases in full-length FMR1 RNA (FIG. 25C). The MALAT1 ASO had no effect on FMR1 isoform levels (FIG. 25C). Next, it was assessed whether FMRP was restored in FXS2 cells following ASO treatment. FIG. 20E shows that 80 nM or 160 nM of ASOs 713 and 714 restored FMRP when compared to TD levels. Therefore, ASO treatment of cells from FXS individuals with a transcriptionally active FMR1 suggests a therapeutic path forward through FMRP restoration (FIG. 20F).

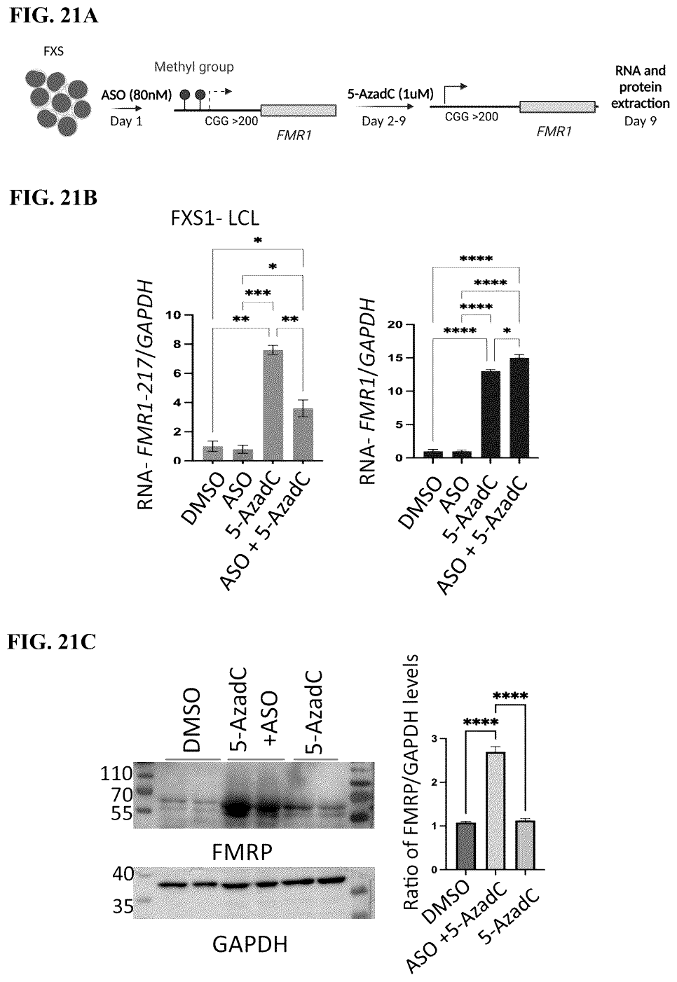

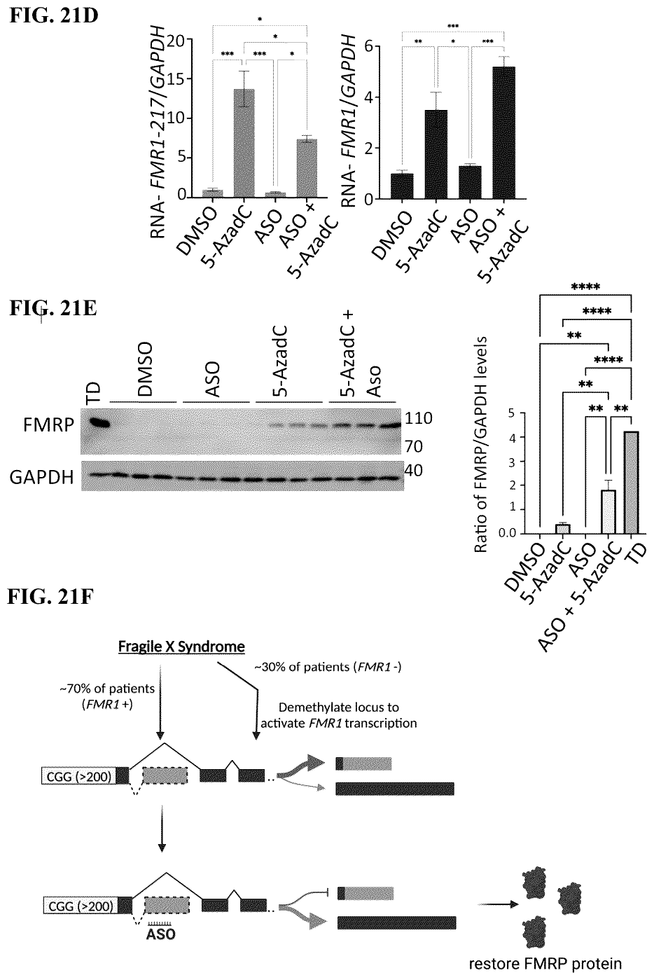

DNA methylation of the CpG island upstream of the FMR1 gene promoter in FXS individuals contributes to transcriptional silencing and loss of FMRP. FMR1 transcription can be reactivated by treatment with 5-AzadC (Tabolacci et al., Transcriptional reactivation of the FMR1 Gene. A possible approach to the treatment of the fragile X syndrome, Genes (Basel) 7:1-16 (2016); Tabolacci et al., Genome-wide methylation analysis demonstrates that 5-aza-2-deoxycytidine treatment does not cause random DNA demethylation in Fragile X syndrome cells, Epigenetics Chromatin 9:1-16 (2016)) and can result in FMR1-217 expression (FIGS. 19C-19D). It was investigated whether in transcriptionally silent FXS cells, reactivation of FMR1 using 5-AzadC together with ASO treatment could restore FMRP expression. To do so, the fully methylated FXS1 LCLs were incubated with either 5-AzadC alone or with 80 nM each of ASOs 713 and 714, 24 hours prior to the addition of the analog. After 7 days of incubation, the samples were collected and analyzed (FIG. 21A). Treatment with the ASOs alone did not affect FMR1 isoform levels because the locus was completely methylated (FIG. 21). Treatment with 5-AzadC for 1 week resulted in the expression of FMR1-217 and full-length FMR1 but led to a modest increase in FMRP levels (FIGS. 21A-21C and FIG. 25D). A longer treatment time or a higher concentration of 5-AzadC may further induce FMRP expression. An earlier study showed that a 36-day treatment of FXS LCLs with 5-AzadC restored FMR1 RNA only up to 40% but produced an even lower level of FMRP compared to that in TD cells (Tabolacci et al., Epigenetics Chromatin 9:1-16 (2016)). Interestingly, a combinatorial treatment of 5-AzadC and ASOs (713 and 714) rescued FMR1-217 RNA levels and further increased the full-length FMR1 compared to 5-AzadC treatment alone (FIG. 21). Although FMRP levels were modestly affected by 5-AzadC alone, a combination of 5-AzadC and the ASOs was more effective in restoring FMRP as compared to that in TD cells (FIGS. 21C and 25D). These data show that in FXS patient-derived cells with full methylation, a combinatorial treatment of demethylation (5-AzadC treatment) and ASOs may restore FMRP.

Finally, two FXS patient-derived fibroblast cell lines were incubated with 5-AzadC and the ASOs, and rescue of FMR1 splicing and restoration of FMRP were determined. A dermal cell line from an FXS individual (GM05131b), with CGG repeat numbers of 800,166 (Sheridan et al., Epigenetic characterization of the FMR1 gene and aberrant neurodevelopment in human induced pluripotent stem cell models of fragile X syndrome, PLoS One 6(10):e26203 (2011)) and previously shown to harbor a transcriptionally active FMR1 locus, was treated with 5-AzadC and then ASOs 713/714 for 72 hours before RNA and protein extraction (FIG. 25E). RT-qPCR of FMR1 and FMR1-217 showed an ASO-dependent decrease in FMR1-217 and a subsequent increase in FMR1 levels (FIG. 25E). The western blot in FIG. 25F shows that while 5-AzadC treatment had no effect on FMRP levels, the ASOs alone or in combination with 5-AzadC significantly increased FMRP levels. In a similar experiment with lung fibroblasts from another FXS individual with a fully methylated FMR1 locus, incubation with 5-AzadC in the absence or presence of ASOs 713/714 resulted in increased FMR1 and FMR1-217 (FIG. 21D). The western blot in FIG. 21E shows that, as with the dermal fibroblasts, ASO treatment resulted in a significant increase of FMRP, albeit lesser than that in the TD fibroblast line.

To summarize, in most FXS patient samples tested, the FMR1 locus was active but predominantly expressed a mis-spliced FMR1-217 isoform as well as very modest levels of FMRP. In the FXS cells that were transcriptionally silent, application of demethylating agents induced FMR1 transcription, which resulted in FMR1-217 expression. In both cases, treatment of cells with ASOs to block FMR1-217 production resulted in partial to complete restoration of FMRP (FIG. 21F).

Defects in alternative splicing of mRNAs alter the transcript and protein repertoire of cells and occur in neurological disorders such as autism, schizophrenia, and bipolar disorder (Gandal et al., Science 362(6240):eaat8127 (2018); Irimia et al., Cell 159:1511-1523 (2014); Quesnel-Vallières et al., Mol. Cell 64:1023-1034 (2016)). In Fmr1 knockout mice, hundreds of alternative splicing events were dysregulated (Shah et al., FMRP Control of Ribosome Translocation Promotes Chromatin Modifications and Alternative Splicing of Neuronal Genes Linked to Autism, Cell Rep. 30(13):4459-72 (2020)). Here, >1,000 RNA mis-splicing events in human FXS WBCs were detected. The large number of WBC RNA changes, if correlated with certain pathologies of FXS, might be useful to assess therapeutic outcomes, disease prognosis, and cognitive abilities (Zafarullah et al., Brain Sci. 9(50:96 (2019); Westmark, Mol. Cell. Pediatr. 1(1):1 (2014); Berry-Kravis et al., Outcome measures for clinical trials in Fragile X syndrome, J. Dev. Behav. Pediatr. 34:508-522 (2013)).

When it contains>200 CGG repeats, the FMR1 gene promoter was methylated and transcriptionally silenced. Surprisingly, FMR1 RNA was detected in 19 of 29 FXS blood samples and in 5 of 10 FXS postmortem brain samples. Several of these FXS individuals harbor FMR1 alleles with >200 CGG repeats and were fully methylated. Remarkably, in >70% of these FXS cells and tissues, the FMR1 RNA was also mis-spliced to generate the FMR1-217 isoform, a truncated RNA that could encode a 31 amino acid peptide. FMR1-217 RNA was not detected in any TD sample. Moreover, in FXS individuals with a fully methylated and silenced FMR1 locus, abrogation of DNA methylation by 5-AzadC treatment resulted in FMR1-217 expression. These data indicate that FMR1 mis-splicing to generate the FMR1-217 isoform in FXS requires a CGG expansion. For example, FMR1-217 RNA expression was detected in FXS premutation carrier-derived fibroblasts with 140 CGG repeats, but not lesser amounts (77 or 98 CGG repeats) or cells from TD individuals (<55 CGG repeats).

These data show that although total FMR1 levels are similar in UFM (partially methylated) FXS2 lymphoblast cell lines (LCLs) to that of the TD LCLs, FMRP expression is much lower. Likewise, high FMR1 expression does not ensure proper FMRP levels in FXS brain tissue samples 1031-09LZ and UMB5746 (Tran et al., Widespread RNA editing dysregulation in brains from autistic individuals, Nat. Neurosci. 22(1):25-36 (2019); Raj et al., Cell Rep. 35:108991 (2021)). Similarly, in FXS LCLs and fibroblasts treated with 5-AzadC, a robust increase in FMR1 RNA, but not FMRP, ensued. Interestingly, all FXS samples that normally express FMR1 full-length RNA, or after 5-AzadC-mediated transcriptional activation, the FMR1-217 mis-spliced RNA was expressed. This relationship between aberrant FMR1 expression in FXS cells and FMR1-217 was also evident in FXS iPSC-derived cells. Although reanalysis of an RNA-seq dataset from FXS neurons with a full CGG expansion showed that FMR1-217 was not produced, they did so when the FMR1 gene was specifically targeted for demethylation by CRISPR/inactive Cas9 fused to Tet1 demethylase (Liu et al., Rescue of Fragile X Syndrome Neurons by DNA Methylation Editing of the FMR1 Gene, Cell 172:979-91 (2018) and FIGS. 19E-19F). A critical point is that while FMR1-217 was generated in FXS iPSC-derived NPCs incubated with 5-AzadC, it was not produced when the CGG expansion was deleted by CRISPR/Cas9 (Vershkov et al., FMR1 Reactivating Treatments in Fragile X iPSC-Derived Neural Progenitors In Vitro and In Vivo, Cell Rep. 26(10):2531-2539 (2019) and FIGS. 19C-19D). Therefore, the CGG expansion drives mis-spliced FMR1-217 generation.

Intellectual impairment is a characteristic of FXS. Measurements of leukocyte full-length FMR1-205, FMR1-217, FMRP, and FMR1 gene methylation performed herein allowed correlation of these molecular parameters with IQ. FMRP was moderately correlated with a higher IQ, whereas FMR1-217 was weakly correlated with a lower IQ. Whether abrogating FMR1-217 RNA could elevate FMR1 and restore FMRP levels was considered. It was found that ASOs targeting the second exon of the FMR1-217 RNA reduced its levels in FXS cells, rescued full-length FMR1, and importantly restored FMRP levels similar to TD cells. Therefore, in a subset of FXS individuals that express FMR1-217, ASO treatment may be a viable therapeutic option. In individuals with a fully methylated FMR1 locus, an ASO-based treatment would be more complex. Consider that in FXS cells with a silenced FMR1, demethylation of the locus by a chemical compound or a demethylating enzyme (Vershkov et al., FMR1 Reactivating Treatments in Fragile X iPSC-Derived Neural Progenitors In Vitro and In Vivo, Cell Rep. 26(10):2531-2539 (2019); Tabolacci et al., Epigenetics Chromatin 9:1-16 (2016); Chiurazzi et al., In vitro reactivation of the FMR1 gene involved in fragile X syndrome, Hum. Mol. Genet. 7:109-113 (1998)) has met with limited success in restoring FMRP. CRISPR/Cas9-mediated gene editing of the CGG repeats (Graef et al., Partial FMRP expression is sufficient to normalize neuronal hyperactivity in Fragile X neurons, Eur. J. Neurosci. 51:2143-2157 (2020); Haenfler et al., Targeted reactivation of FMR1 transcription in Fragile X Syndrome embryonic stem cells, Front. Mol. Neurosci. 11:282, (2018); Xie et al., Reactivation of FMR1 by CRISPR Cas9-mediated deletion of the expanded CGG-repeat of the fragile X chromosome, PLoS One 11:1-12 (2016); Park et al., Reversion of FMR1 methylation and silencing by editing the triplet repeats in Fragile X iPSC-derived Neurons, Cell Rep. 13:234-241 (2015)) has resulted in a nearly 70% restoration of FMRP levels. However, it is shown herein that in FXS cells with silenced FMR1, DNA demethylation combined with ASO treatment restores FMRP. Therefore, treatments that combine DNA demethylation with a splice-switching ASO might be a useful therapeutic strategy for individuals with a fully silenced FMR1 gene. In this study, a proof of concept has been presented in which splice-switching ASOs can restore FMRP levels in FMR1-expressing FXS cells. FMR1-217 RNA, which is expressed in FXS human brain tissues as well iPSC-derived neurons, may respond to treatment with splice-switching ASOs and restore FMRP.

Recent advances have shown the clinical feasibility of using ASOs to treat neurological disorders such as spinal muscular atrophy (SMA) (Finkel et al., Treatment of infantile-onset spinal muscular atrophy with nusinersen: A phase 2, open-label, dose-escalation study, Lancet 388:3017-3026 (2016)), myotonic dystrophy (Mulders et al., Triplet-repeat oligonucleotide-mediated reversal of RRNA toxicity in myotonic dystrophy, Proc. Natl. Acad. Sci. U.S.A. 106:13915-13920 (2009); Pandey et al., Identification and characterization of modified antisense oligonucleotides targeting DMPK in mice and nonhuman primates for the treatment of myotonic dystrophy type 1 s, J. Pharmacol. Exp. Ther. 355:329-340 (2015); Wheeler et al., Targeting nuclear RNA for in vivo correction of myotonic dystrophy, Nature 488:111-115 (2012)), ALS (amyotrophic lateral sclerosis) (Smith et al., Antisense oligonucleotide therapy for neurodegenerative disease, J. Clin. Invest. 116:2290-2296 (2006); Donnelly et al., RNA toxicity from the ALS/FTD C9ORF72 expansion is mitigated by antisense intervention, Neuron 80:415-428 (2013); Tran et al., Suppression of mutant C9orf72 expression by a potent mixed backbone antisense oligonucleotide, Nat. Med. 28:117-124 (2022); Becker et al., Therapeutic reduction of ataxin-2 extends lifespan and reduces pathology in TDP-43 mice, Nature 544:367-371 (2017); Jiang et al., Spinal morphine but not ziconotide or gabapentin analgesia is affected by alternative splicing of voltage-gated calcium channel CaV2.2 pre-mRNA, Mol. Pain 9:67 (2013)), and Angelman syndrome (Milazzo et al., Antisense oligonucleotide treatment rescues UBE3A expression and multiple phenotypes of an Angelman syndrome mouse model, JCI Insight 6:e145991 (2021); Dindot et al., An ASO therapy for Angelman syndrome that targets an evolutionarily conserved region at the start of the UBE3A-AS transcript, Sci. Transl. Med. 15:abf4077 (2023)). The findings disclosed herein suggest that ASOs can correct dysregulated alternative splicing of FMR1 and restore FMRP in individuals with FXS, thereby offering a unique therapeutic strategy to treat the disorder.

### Materials and Methods

### Human FXS Participant Studies.

FXS male patients (CGG repeats>200) between the ages of 16 to 38 years with FXS phenotypes, IQ range (obtained using the Stanford-Binet Scale Fifth Edition (SB5) (Roid et al., Contemporary Intellectual Assessment: Theories, Tests, and Issues, The Guilford Press, New York, NY, 3:249-268 (2012)) and ABC (The Adaptive Behavior Composite) standard score) were measured (Tables 9-12 and FIGS. 26A-27B). Samples from age-matched TD males (CGG repeats<55) with a normal IQ and no known neuropsychiatric conditions were obtained (Tables 9-12 and FIGS. 26A-27B). All participants or their legal guardians, as appropriate, signed informed consent to the study. The project was approved by the Rush University Medical Center's Institutional Review Board. Methylation status was determined using the Asuragen (Austin, TX) FMR1 methylation PCR Kit and/or southern blot analysis. FMRP levels were quantified by generating dried blood spots (DBS) or by using peripheral blood mononuclear cell (PBMC) samples. Detailed protocols are described in the Supplemental Methods.

Frozen postmortem brain tissues were obtained from the University of California at Davis Brain Repository from FXS male individuals (N=6) and age-matched TD males (N=5).

RNA Extraction and Sequencing of Tissue Samples from FXS and TD Individuals.

### Leukocytes.

Fresh blood (8 mL) was collected (See Tables 9-12, FIGS. 26A-27B, and Supplemental Methods for details). RNA extraction was performed using TRIZOL® (biological material isolating reagent) LS Reagent (Thermo Fisher Scientific, Waltham, MA; #10296028). The RNA obtained was then DNase-treated with TURBO™ DNase (Invitrogen, Waltham, MA; #AM2238) and cleaned using the RNA clean and concentrator kit (Zymo Research, Irvine, CA: #11-325). The quality of RNA (RIN, RNA integrity number>7.3, Tables 9-12 and FIGS. 26A-27B) was assessed using fragment analysis. RNA sample (3 μg) was used for directional mRNA library preparation using polyA enrichment (Novogene, Durham, NC) and the libraries were sequenced on the NovaSeq platform to generate paired-end, 150 bp reads at a sequencing depth of 60 to 90 million reads per sample (Tables 9-12 and FIGS. 26A-27B).

### Brain Tissue.

RNA was extracted from powdered postmortem frozen cortical tissues using TRIZOL® Reagent (Thermo Fisher Scientific #15596026), and the lysate was collected. Total RNA was extracted using bromo-3-chloro-propane (BCP), recovered as above, and stored at −80° C.

### ASO Synthesis and Treatment.

ASOs were synthesized on a Dr. Oligo 48 synthesizer (Biolytic, Fremont, CA). 2′-O-methoxyethyl (MOE)-modified phosphoramidites were coupled for 8 minutes. Oligonucleotides were deprotected in concentrated aqueous ammonia (30% in water) at 55° C. for 16 hours and characterized by liquid chromatography-mass spectrometry. Final desalting was effected by diafiltration (3×water wash) in a 3-kDa cutoff Amicon centrifugal filter. ASOs were added individually or in combinations to LCL cell lines or fibroblast cultures at a final concentration of 80 nM or 160 nM using Lipofectamine RNAIMAX® Transfection Reagent (Thermo Fisher Scientific, 13778030). The cells were collected after 72 hours of ASO treatment for RNA and protein extraction.

### 5-AzadC Treatment.

5-Aza-2′-deoxycytidine (5-AzadC) (Sigma-Aldrich, St. Louis, MO; A3656) was added to the cell cultures (final concentration 1 μM) for 7 consecutive days. For samples with both 5-AzadC and ASO treatment, 80 nM or 160 nM ASOs or vehicle was added on day 1 and either 5-AzadC or DMSO was added each day from day 2 up to day 9 at a final concentration of 1 μM. On day 9, the cells were collected in 1×phosphate-buffered saline to proceed with RNA extraction or western blotting (See Supplemental Methods further details).

### Data, Materials, and Software Availability.

Certain previously published data were used for this work (See NCBI GEO (Gene Expression Omnibus; https://www.ncbi.nlm.nih.gov/geo) GSE117776 (Tran et al., Widespread RNA editing dysregulation in brains from autistic individuals, Nat. Neurosci. 22(1):25-36 (2019)), GSE112145 (Vershkov et al., FMR1 Reactivating Treatments in Fragile X iPSC-Derived Neural Progenitors In Vitro and In Vivo, Cell Rep. 26(10):2531-2539 (2019)), and GSE108498 (Liu et al., Rescue of Fragile X Syndrome Neurons by DNA Methylation Editing of the FMR1 Gene, Cell 172:979-91 (2018))). All analyses were performed using the DolphinNext platform (Kucukural et al., DolphinNext: A graphical user interface for creating, deploying and executing Nextflow pipelines, J Biomol Tech. 31:S25 (2020)). Datasets generated in this study have been deposited into the Gene Expression Omnibus (GEO) database under the accession number: Super series GSE202179 (Shah et al., Antisense Oligonucleotide Rescue of CGG Expansion-Dependent FMR1 Mis-Splicing in Fragile X Syndrome Restores FMRP, Proc Natl Acad Sci USA (2023)).

### Supplemental Methods

### Human FXS Participant Studies

All participants were Caucasian males with a FMR1 full mutation (CGG repeats>200) or typically developing individuals (CGG repeats<55) as confirmed by DNA analysis. All participants or their legal guardians, as appropriate, signed informed consent to the study. The project was approved by the Rush University Medical Center Institutional Review Board. Intelligence quotient (IQ) scores were obtained using the Stanford-Binet Scale—Fifth Edition (SB5) (Roid et al., Contemporary Intellectual Assessment: Theories, Tests, and Issues, The Guilford Press, New York, NY, 3:249-268 (2012)) and applying the zdeviation method to avoid floor effects in persons with intellectual disability (Sansone et al., Improving IQ measurement in intellectual disabilities using true deviation from population norms, J Neurodev Disord. 6(1):16 (2014)). The adaptive skills of participants were determined using an semi-structured interview and measured using the Vineland Adaptive Behavior skills (Vineland-3, (Sparrow et al., Vineland adaptive behavior scales, Am. Guid. Serv., Circle Pines, MN (1984))). The Adaptive Behavior Composite (ABC) standard score (SS) is the measure of overall adaptive functioning based on scores assessing the following domains: communication, daily living skills, and socialization. FXS patients were aged 16-38 years with FXS phenotypes, a z-deviation IQ range of 20-52 and ABC standard score range of 20-41 (Tables 9-12 and FIGS. 26A-27B). Age matched TD individuals for the study were aged 22-29 with a normal IQ and no known neuropsychiatric conditions (Tables 9-12 and FIGS. 26A-27B). For CGG repeat size determination in the 5′ UTR of the FMR1 gene, DNA isolated from whole blood was analyzed using the Asuragen FMR1 AMPLIDEX® PCR Kit. Methylation status was determined using the Asuragen FMR1 methylation PCR Kit and/or Southern blot analysis. FMRP levels were quantified by generating dried blood spots (DBS) from the samples. FMRP levels were quantified by generating dried blood spots (DBS) from the samples. To generate DBS, twelve 50 mL spots were put on each blood card and allowed to dry. The blood cards were then stored at −80° C. Discs were punched using a 6 mm punch and incubated in lysis buffer. Extracted sample was centrifuged and FMRP quantified using the LUMINEX® Microplex immunochemistry assay. FMRP levels were normalized to 1000 white blood cells (WBCs) per sample. Additionally, FMRP levels were also quantified by using peripheral blood mononuclear cells (PBMC) samples. PBMC were isolated from whole blood using a Cell Preparation (CPT) blood tube. Isolated PBMC were lysed and quantified for total protein concentration using a spectrophotometer and FMRP quantified using a LUMINEX® Microplex immunochemistry assay. FMRP levels were normalized to total protein. Both methods produced comparable levels of FMRP in the samples assessed.

Frozen post-mortem brain tissues were obtained from University of California at Davis Brain Repository from FXS male individuals (N=6) and age-matched typically developing (TD) males (N=5).

RNA Extraction and Sequencing of Tissue Samples from FXS and TD Individuals

### Leukocytes

Eight mL fresh blood were collected from FXS male individuals (N=29) and age-matched typically developing (TD) males (N=13) (See Tables 9-12 and FIGS. 26A-27B) in a BD VACUTAINER® CPT (Cell Preparation Tube with sodium citrate-blue top tube, Becton Dickinson and Company (BD), Franklin Lakes, NJ; #REF362761) and the leukocytes collected on a LeukoLOCK™ filter prior to RNA extraction using a LeukoLOCK™ Fractionation & Stabilization Kit (Ambion, Waltham, MA; #1933) as per the manufacturer's instructions. Briefly, the blood samples were passed through LeukoLOCK™ filters that were then rinsed with 3 mL phosphate buffered saline (PBS) followed by 3 mL of RNALATER® tissue storage reagent and RNA protectant. The residual RNALATER® was expelled from the LeukoLOCK™ filter and the filters were capped and stored in −80° C. To extract RNA, the filters were thawed at room temperature for 5 minutes and then the remaining few drops of RNALATER® were removed. The filter was flushed with 4 mL of TRIZOL® LS Reagent (Thermo Fisher Scientific #10296028), and the lysate was collected in a 15 mL tube. A volume of 800 μl bromo-3-chloro-propane (BCP) (Sigma-Aldrich #B9673) was added to each tube and vortexed vigorously for 30 seconds. The tube was then incubated at room temperature for 5 minutes and centrifuged for 10 minutes at 4° C. at about 2,000×g; the aqueous phase containing the RNA was recovered. To recover the long RNA fraction, 0.5 volumes of 100% ethanol were added and mixed well. The RNA was then recovered using an RNA clean and concentrator kit (Zymo Research, Irvine, CA; #11-325/R1015), DNase-treated with TURBO™ DNase (Invitrogen #AM2238) and the RNA resuspended in RNase-free water and stored at −80° C. The quality of RNA (RIN, RNA integrity number>7.3, Tables 9-12 and FIGS. 26A-27B) was assessed using a 5300 Fragment Analyzer instrument. Three mg of RNA sample was used for directional mRNA library preparation using polyA enrichment (Novogene), and the libraries were sequenced on the NovaSeq platform to generate paired end, 150 bp reads at a sequencing depth of 60-90 million reads per sample (Tables 9-12 and FIGS. 26A-27B).

### Brain Tissue

The post-mortem frozen cortical tissues from FXS male individuals (N=6) and age-matched typically developing (TD) males (N=5) were powdered in liquid nitrogen using a mortar and pestle. The fine powder was then homogenized on ice in a dounce homogenizer using TRIZOL® Reagent (Thermo Fisher Scientific #15596026), and the lysate was collected. Total RNA was extracted using BCP and recovered as above and stored at −80° C.

cDNA Synthesis and qPCR

One μg of total RNA was primed with oligo(dT)₂₀ primer to generate cDNA with a QUANTITECT® cDNA synthesis kit (Qiagen, Germantown, MD; #205311) using random hexamers or OligodT priming (FIG. 23B). qPCR was performed using the iTaq™ Universal SYBR® Green Supermix (Bio-Rad, Hercules, CA; #1725122) on a QuantStudio 3 qPCR machine in duplicate. For each experiment, three technical replicates were included, and the experiment was repeated at least twice.

### RNA-Seq Data Analysis

Fastq files were uploaded to the DolphinNext platform (Yukselen et al., DolphinNext: A distributed data processing platform for high throughput genomics, bioRxiv 689539 (2019)) at the UMass Chan Medical School Bioinformatics Core for mapping and quantification. The reads were subjected to FastQC (v0.11.8) analysis, and the quality of reads was assessed. Reads were mapped to the genome assembly GRCh38 (hg38) version 34 using the STAR (v2.5.3a) aligner. Gene and isoform expression levels were quantified by Salmon v1.5.2. Transcript names were assigned using GENCODE/Ensembl V43.

Differential gene expression analysis: DESeq2 (v3.9) was used to obtain differentially expressed genes from the estimated counts table. After normalization by the median of ratios method, genes with minimal 5 counts average across all samples were kept for the Differential Gene expression analysis. The P<0.0002 was used as a cutoff. The TDF files generated were uploaded on the Integrative Genomics Viewer (2.6.2) and auto scaled for visualization.

Alternative splicing analysis: To analyze differential alternative splicing (AS), the rMATS package v3.2.5 (Shen et al., rMATS: Robust and flexible detection of differential alternative splicing from replicate RNA-Seq data, Proc. Natl. Acad. Sci. 111(51):E5593-5601 (2014)) was used with default parameters. The Percent Spliced In (PSI) levels or the exon inclusion levels calculated by rMATS using a hierarchical framework. To calculate the difference in PSI between genotypes a likelihood-ratio test was used. AS events with an FDR<5% and |deltaPSI|≥5% as identified using rMATS were used for further analysis. The genes with significant skipped exons were used for validation using RT-qPCR analysis. One mg of RNA was used to generate cDNA using the QUANTITECT® cDNA synthesis kit. Primers were designed to overlap skipped/inclusion exon junctions and qPCR was performed using the Bio-Rad SYBR® reagent on a QuantStudio 3 instrument.

### Cell Culture

### Lymphoblast Cell Lines

Lymphoblast cell lines (LCL) were obtained from Coriell Institute from two FXS individuals (GM07365 (FXS1), GM06897(FXS2)) and two typically developing control males (GM07174 (WT3), GM06890 (WT4)). Cells were cultured in RPMI 1640 medium (Sigma-Aldrich), supplemented with 15% fetal bovine serum (FBS) and 2.5% L-glutamine at 37° C. with 5% CO₂ in T25 flasks.

### Fibroblast Cells

Skin biopsies from participants were collected in a 15-cc tube with transfer culture medium (DMEM with 5% Gentamicin). The biopsy was then removed from the transfer medium with tweezers onto a sterile tissue culture dish and dissected into approximately 6-7 pieces using sterile tweezers and scissors in the culture hood. Three to four pieces of skin explants were kept on the bottom of a T25 flask and 3 mL CHANG AMNIO® culture medium was added. The flask was then incubated at 37° C. with 5% CO₂ for 10 days. The culture medium was changed after cells started growing out from the skin explants. After the cells had grown to 5-6 layers around the skin explants, the skin explants were removed from the culture flask and fibroblasts were trypsinized and spread evenly in the flask. The media were changed after overnight incubation with trypsin. Fibroblast culture medium was added (complete medium (500 mL DMEM (15-017-CV) with 10% FBS and 1×antibiotic/antimitotic, 1×L-glutamine 5 mL)) twice a week to cells in a T25 culture flasks at 37° C. with 5% CO₂. Fibroblast cell lines were obtained from Coriell Institute from two FXS individuals (GM05131 and GM07072). A control fibroblast line derived from a skin sample of a typically developing male was used. Cells were cultured in DMEM medium (Sigma-Aldrich), supplemented with 10% fetal bovine serum (FBS) and 2.5% L-glutamine at 37° C. with 5% CO₂.

### ASO Synthesis and Treatment

### ASO Synthesis

ASOs were synthesized according to standard guidelines for design of exon skipping ASOs (Shah et al., FMRP Control of Ribosome Translocation Promotes Chromatin Modifications and Alternative Splicing of Neuronal Genes Linked to Autism, Cell Rep. 30(13):4459-72 (2020)) on a Dr. Oligo 48 synthesizer. 2′-O-methoxyethyl (MOE)-modified phosphoramidites were coupled for 8 minutes. Oligonucleotides were deprotected in concentrated aqueous ammonia (30% in water) at 55° C. for 16 hours and characterized by liquid chromatography-mass spectrometry. Final desalting was effected by diafiltration (3×water wash) in a 3-kDa cutoff Amicon centrifugal filter.

### ASO Treatment

Antisense oligonucleotides (ASOs) were dissolved in ultrapure distilled water to a final concentration of 10 μM. Before use, the ASOs were heated to 55° C. for 15 minutes and cooled at room temperature. ASOs were added individually or in combinations to LCL cell lines at a final concentration of 80 nM or 160 nM using Lipofectamine RNAIMAX® Transfection Reagent (Thermo Fisher Scientific, 13778030) and incubated at 37° C. with 5% CO₂ for 16 hours in reduced serum medium. RPMI 1640 medium (Sigma-Aldrich), supplemented with 15% fetal bovine serum (FBS), was added for a total of 72 hours. The cells were collected after 72 hours of ASO treatment for RNA and protein extraction.

### 5-AzadC Treatment

For each cell culture, 30×10⁵ cells/mL were added to a final volume of 20 mL medium (RPMI 1640 medium (Sigma-Aldrich) supplemented with 15% fetal bovine serum (FBS) and 2.5% L-glutamine at 37° C. with 5% CO₂) per T25 flask. 5-Aza-2′-deoxycytidine (5-AzadC) (Sigma-Aldrich, A3656) was added to the cell cultures (final concentration 1 PM) for 7 consecutive days. A 2 mM stock of 5-AzadC was made in dimethyl sulfoxide (DMSO). For each cell line, two independent treatments were performed (n=2). For the no treatment controls for each cell line, DMSO was added to the flasks. For samples with both 5-AzadC and ASO treatment, 80 nM or 160 nM ASOs or vehicle were added on Day 1 and either 5-AzadC or DMSO was added each day from Day 2 up to Day 9 at a final concentration of 1 μM. On Day 9, the cells were collected in 1×phosphate buffered saline to proceed with RNA extraction or Western blotting.

### Western Blotting

Cells were homogenized at 4° C. in radioimmunoprecipitation assay (RIPA) lysis buffer with incubation on ice for 10 minutes and dissociation by pipetting. The extract was centrifuged at 13,200 rpm for 10 minutes at 4° C., and the supernatant was collected. Protein concentration was determined by BCA reagent. Proteins (20 μg) were diluted in sodium dodecyl sulfate (SDS)-bromophenol blue reducing buffer with 40 mM dithiothreitol (DTT) and analyzed using western blotting on a 10% SDS polyacrylamide gel electrophoresis (PAGE) gel with the following antibodies: FMRP (MilliporeSigma, Burlington, MA, mAb2160, 1: 1000), FMRP (Abcam, Waltham, MA, ab17722, 1:1000) and GAPDH (14C10, Cell Signaling Technology, Danvers, MA, mAb 2118, 1:2000) diluted in 1×tris-buffered saline with Tween 20 (TBST) with 5% non-fat milk. Membranes were washed three times for 10 minutes with 1×TBST and incubated with anti-rabbit or anti-mouse secondary antibodies (Jackson ImmunoResearch Inc., West Grove, PA, 1:10000) at room temperature for 1 hour. Membranes were washed three times for 10 minutes with 1×TBST, developed with Pierce™ ECL-Plus Western Blotting Substrate, and scanned with a GE Amersham Imager.

### Quantification and Statistical Analysis

All grouped data are presented as mean±standard error of the mean (s.e.m.). All tests used to compare the samples are mentioned in the respective Figure legends and corresponding text. When exact P values are not indicated, they are represented as follows: *, p<0.05; **, p<0.01; ***, p<0.001; ****, P value<0.0001; not significant (n.s.), p>0.05.

### Chromatin Immunoprecipitation Sequencing (ChIP-Seq)

Eight mL fresh blood was collected from FXS male (N=3) and age-matched typically developing males (N=2) individuals in a BD VACUTAINER® CPT (Cell Preparation Tube with sodium citrate-blue top tube, Becton Dickinson and Company (BD) #REF362761). The tube was gently inverted 5 times and the sample was centrifuged for 25 minutes at 1500-1800 relative centrifugal force (RCF) at room temperature. The tubes were then inverted to collect the lymphocytes and other mononuclear cells resuspended in the upper liquid phase in a new 15 mL tube. The samples were centrifuged again for 10 minutes at 300 RCF to obtain the PBMC pellet. The PBMCs were rinsed with 1× Dublecco's phosphate buffered saline without calcium or magnesium (D-PBS) (Invitrogen #14190-094). The PBMC pellet was resuspended in 250 μL ice-cold D-PBS with protease inhibitors. FMRP levels in PBMCs were quantified using a LUMINEX® Microplex immunochemistry assay. Chromatin isolation and sequencing were performed as previously described (Shah et al., FMRP Control of Ribosome Translocation Promotes Chromatin Modifications and Alternative Splicing of Neuronal Genes Linked to Autism, Cell Rep. 30(13):4459-72 (2020)). Briefly, the cells were cross-linked with 1% formaldehyde and quenched with 150 mM glycine. After centrifugation at 2000×g for 10 minutes at 4° C., the cells were lysed. After homogenization, the nuclei were harvested by centrifugation at 2000×g for 5 minutes at 4° C. The nuclei were lysed by incubating for 20 minutes on ice in nuclear lysis buffer (10 mM tris(hydroxymethyl)aminomethane (Tris) (pH 8.0), 1 mM ethylenediaminetetraacetic acid (EDTA), 0.5 mM ethylene glycol-bis(β-aminoethyl ether)-N,N,N′,N′-tetraacetic acid (EGTA)). 0.5% SDS was added, and the samples were sonicated on a BioruptorR sonicator at high power settings for 9 cycles (sonication: 30 seconds on, 90 seconds off) of 15 minutes each at 4° C. The samples were centrifuged and diluted to adjust the SDS concentration to <0.1%. 10% of each sample was used as input. The remainder of the samples were divided into two and incubated with protein G DYNABEADS® coupled overnight at 4° C. with antibodies against H3K36me3 (Abcam ab9050, 5 μg per ChIP) or H3K4me3 (Active Motif, Carlsbad, CA 39159, 5 μg per ChIP). After IP, the beads were washed and chromatin and de-crosslinked overnight at 65° C. After RNase and proteinase K treatment, the DNA was purified. ChIP-Seq libraries were prepared by performing the following steps ends repair using T4 DNA polymerase, A′ base addition by Klenow polymerase and Illumina adapter ligation using T4 Polynucleotide kinase from New England Biolabs (NEB, Ipswich, MA). The library was PCR amplified using multiplexing barcoded primers. The libraries were pooled with equal molar ratios, denatured, diluted, and sequenced with NextSeq 500/550 High Output Kit v2.5 (Illumina, San Diego, CA, 75 bp paired-end runs) on a Nextseq500 sequencer (Illumina).

### ChIP-Seq Analysis

For ChIP-seq data analysis, alignments were performed with Bowtie2 (2.1.0) using the GRCh38 (hg38) version 34 genome, duplicates were removed with Picard, and TDF files for IGV viewing were generated using a ChIP-seq pipeline from DolphinNext (Yukselen et al., DolphinNext: A distributed data processing platform for high throughput genomics, bioRxiv 689539 (2019)). The broad peaks for H3K36me3 ChIP-Seq were called using the broad peak parameter MACS2. Narrow peaks for H3K4me3 ChIP were called using the narrow parameter in MACS2. deepTools2 (Ramirez et al., deepTools2: a next generation web server for deep-sequencing data analysis, Nucleic Acids Res. 44:W160-W165 (2016)) was used to plot heatmaps and profiles for genic distribution of H3K36me3 and H3K4me3 ChIP signals over input. IGV tools (2.6.2) were used for visualizing TDF files, and all tracks shown were normalized for total read coverage.

Dataset S1 (Tables 9-12 and FIGS. 26A-27B): Includes details regarding FXS and TD human samples, RNA-Sequencing read depth and mapping information, Antisense Oligonucleotides (ASO) sequences, Primer sequences for those used in qPCR, FMR1 transcript levels for all RNA-seq samples. Dataset S2 (Tables 13-19): Includes RNA level changes from the DGE analysis and clustering analysis (SI Appendix, FIG. 22A). DAS changes related to FIGS. 17B and 17C. Dataset S3 (Tables 20-28): DAS and DGE analysis data from FIGS. 19A-19H.

TABLE 9
Sample Information

| | | | | | | | | FMRP levels | | | | | | |
|---|---|---|---|---|---|---|---|---|---|---|---|---|---|---|
| | | | | | | | | PMBC [ng | DBS | | | | | |
| | | | | | | | | FMRP/ug | [FMRP(ng/ | | | | | |
| | Sample | | | | | SB | MPCR | total | mL)/1000 | SB5 Z dev | Vineland Seversity Score | | | |
| Lab ID | ID | Gender | Age | Status | CGG | Methylation | Methylation | protein] | WBC] | IQ | ABC | Com | \|DLS | Social |
| FXS01 | A379 | M | 21 | FXS | 140, 175, >200 | — | 140 100% FM, | 6.56E−03 | 1.82E−03 | 37.77 | 20 | 20 | 20 | 20 |
| | | | | | | | 175 3% under methylated, | | | | | | | |
| | | | | | | | >200 FM 90% | | | | | | | |
| FXS02 | A387 | M | 36 | FXS | >200 | X | 81% FM | 2.07E−03 | 2.80E−04 | 26.76 | 21 | 20 | 22 | 20 |
| FXS03 | R14 | M | 18 | | 150, >200 | 5% Unmethylated | N/A | N/A | N/A | 52 | 34 | 32 | 23 | 44 |
| | | | | | | & 95% Fully | | | | | | | | |
| | | | | | | methylated | | | | | | | | |
| FXS04 | R09 | M | 21 | FXS | 102, >200 | N/A | N/A | N/A | N/A | 37 | 20 | 20 | 20 | 20 |
| FXS05 | A457 | M | 41 | FXS | >200 | P | >200 96% fully Methylated | 4.85E−04 | 3.23E−04 | 35.08 | 50 | 54 | 36 | 55 |
| FXS06 | A426 | M | 20 | FXS | 65, >200 | P | 65 2% under methylated, | 1.77E−02 | 1.11E−02 | 55.02 | 73 | 64 | 79 | 78 |
| | | | | | | | >200 100% FM | | | | | | | |
| FXS07 | A365 | M | 19 | FXS | >200 | P | P | 2.28E−04 | <0** | 25.04 | | | | |
| FXS08 | R07 | M | 27 | FXS | 173, >200 | Unmethylated & | | | | 39.9 | 32 | 24 | 51 | 20 |
| | | | | | (~710, ~613) | methylated | | | | | | | | |
| FXS09 | A285 | M | 22 | FXS | >200 | | 100% FM | 4.40E−04 | 4.70E−05 | 34.99 | 32 | 24 | 49 | 20 |
| FXS10 | A377 | M | 30 | FXS | >200 | X | 100% FM | 3.11E−04 | 2.04E−04 | 56.01 | 34 | 32 | 38 | 31 |
| FXS11 | A366 | M | 33 | FXS | >200 | — | 100% FM | 6.50E−03 | N/A | 62.26 | 20 | 20 | 20 | 20 |
| FXS12 | A393 | M | 32 | FXS | >200 | X | 100% FM | 4.85E−04 | 2.16E−04 | 26.91 | 20 | 20 | 20 | 20 |
| FXS13 | A373 | M | 30 | FXS | 102, 174, >200 | FMF | 102, 174 100% FM, >200 100% FM | 5.35E−04 | 1.12E−04 | 27.64 | 21 | 20 | 22 | 20 |
| FXS14 | R15 | M | 16 | FXS | >200 | N/A | N/A | N/A | N/A | 20 | 39 | 34 | 20 | 60 |
| FXS15 | A284 | M | 21 | FXS | >200 | — | 100% FM | 2.56E−04 | 9.13E−05 | 45.91 | 35 | 26 | 40 | 37 |
| FXS16 | A435 | M | 37 | FXS | 63, >200 | — | 63 2% under methylated, | 3.50E−03 | 0.0001578 | 53.5 | 68 | 66 | 59 | 77 |
| | | | | | | | 194 36% partially methylated, | | | | | | | |
| | | | | | | | >200 100% FM | | | | | | | |
| FXS17 | A367 | M | 28 | FXS | >200 | — | 100% FM | 1.05E−04 | <0** | 44.02 | | | | |
| FXS18 | A453 | M | 26 | FXS | >200 | P | P | 1.34E−04 | 2.44E−04 | 30.28 | 20 | 20 | 20 | 20 |
| FXS19 | R12 | M | 35 | FXS | >200 | | | | | 50 | 41 | 22 | 40 | 58 |
| FXS20 | A388 | M | 25 | FXS | >200 | X | 100% FM | 4.85E−04 | 2.66E−04 | 29.64 | 51 | 48 | 49 | 52 |
| FXS21 | A394 | M | 34 | FXS | >200 | X | 100% FM | 2.00E−04 | 2.10E−04 | 37.65 | 36 | 20 | 44 | 43 |
| FXS22 | A292 | M | 29 | FXS | >200 | X | — | 4.88E−04 | 2.20E−04 | 35.82 | 20 | 20 | 20 | 20 |
| FXS23 | A369 | M | 25 | FXS | >200 | — | 94% FM | <0** | <0** | | | | | |
| FXS24 | A372 | M | 23 | FXS | 28**, >200 | FMF | 100% FM | <0** | <0** | 37.58 | 24 | 20 | 30 | 20 |
| FXS25 | A376 | M | ? | FXS | >200 | FMF | 100% FM | <0 | <0 | | 29 | 22 | 20 | 42 |
| FXS26 | A395 | M | 33 | FXS | >200 | — | 100% FM | 4.85E−04 | 2.16E−04 | 41.83 | 27 | 20 | 40 | 20 |
| FXS27 | A420 | M | 19 | FXS | >200 | — | >200 100% FM | 4.85E−04 | 2.70E−04 | 20.2 | 35 | 24 | 27 | 51 |
| FXS28 | R11 | M | | FXS | | | | | | | | | | |
| FXS29 | R13 | M | 38 | FXS | >200 | | >200: 85% methylated | | | 49 | 30 | 20 | 49 | 20 |
| TD01 | A454 | M | 24 | TD | 30 | N/A | N/A | 6.35E−02 | 1.17E−02 | | | | | |
| TD02 | R02 | M | | TD | | | | | | | | | | |
| TD03 | R03 | M | | TD | | | | 0.050422 | 0.012831 | | | | | |
| TD04 | A400 | M | 18 | TD | 39 | N/A | N/A | 4.29E−02 | 1.19E−02 | | | | | |
| TD05 | R04 | M | | TD | | | | | | | | | | |
| TD06 | R05 | | | TD | | | | 0.04193 | 0.012076 | | | | | |
| TD07 | A294 | M | 25 | TD | 30 | N/A | N/A | 4.83E−04 | 1.23E−02 | | | | | |
| TD08 | A293 | M | 26 | TD | 37 | N/A | N/A | 4.40E−04 | 1.72E−02 | | | | | |
| TD09 | A401 | M | 26 | TD | 38 | N/A | N/A | 7.24E−02 | 1.86E−02 | | | | | |
| TD10 | A455 | M | 23 | TD | 20 | N/A | N/A | 9.77E−02 | 1.45E−02 | | | | | |
| TD11 | A307 | M | 34 | TD | 29 | N/A | N/A | 5.17E−02 | 1.87E−02 | | | | | |
| TD12 | A427 | M | 23 | TD | 29 | N/A | N/A | 7.19E−02 | 2.84E−02 | | | | | |
| TD13 | A422 | M | 23 | TD | 29 | N/A | N/A | 8.46E−02 | 2.07E−02 | | | | | |

TABLE 10
Sequence Details

| | | Multimapped | Unique | |
|---|---|---|---|---|
| | | Reads | Reads | |
| | | Aligned | Aligned | |
| Sample | Total Reads | (STAR) | (STAR) | RIN |
| A307 | 119794556 | 6041675 | 111670371 | 9.1 |
| R14 | 68519177 | 2134692 | 64882978 | 9.1 |
| R13 | 62360982 | 2670612 | 58441217 | 8.7 |
| R09 | 69872833 | 1862773 | 66242115 | 7.3 |
| A394 | 43494351 | 4177547 | 38304886 | 4.7 |
| A395 | 65223278 | 2829839 | 60807840 | 5.2 |
| R12 | 69969048 | 2084909 | 66394907 | 9 |
| R05 | 59972242 | 2910442 | 55917208 | 8.3 |
| A453 | 64967407 | 2741029 | 60359847 | 6.4 |
| A422 | 56652712 | 3069764 | 51245265 | 4.9 |
| A367 | 67516939 | 5529074 | 60678125 | 6.3 |
| R07 | 61410724 | 2014705 | 58110688 | 8.2 |
| A455 | 53555660 | 2427253 | 49638699 | 4 |
| R11 | 69057763 | 2266440 | 65253494 | 8.7 |
| A366 | 67188435 | 3317969 | 62525200 | 8.3 |
| R04 | 65397350 | 3573503 | 60430335 | 8.6 |
| A376 | 65197658 | 3461805 | 60255474 | 8.7 |
| A435 | 59988280 | 5313304 | 52703153 | 4 |
| A365 | 66643094 | 1449256 | 63599170 | 7.9 |
| A369 | 64864188 | 1521309 | 61810371 | 7.5 |
| A457 | 68756440 | 2532756 | 64239177 | 6.9 |
| A293 | 129747499 | 3607052 | 124018952 | 9.7 |
| A393 | 61870959 | 4354839 | 56004641 | 5 |
| A373 | 69346473 | 1384983 | 66189257 | 7.4 |
| R02 | 64886909 | 2082376 | 61469383 | 8.7 |
| A294 | 134619931 | 5585971 | 126950003 | 9.4 |
| A285 | 101180767 | 3946740 | 95446571 | 8.5 |
| R15 | 62230963 | 2763217 | 57991216 | 8.9 |
| A388 | 64785498 | 1698146 | 61247919 | 7.9 |
| A284 | 121201356 | 4319244 | 115033465 | 9.7 |
| A427 | 61961032 | 3969130 | 55912405 | 6.4 |
| A379 | 70861804 | 4544217 | 64842742 | 5.7 |
| A426 | 68509163 | 2854422 | 63310021 | 5.5 |
| R03 | 62515654 | 2382873 | 58990362 | 8.6 |
| A400 | 67394598 | 3004462 | 62188035 | 5.8 |
| A454 | 60912400 | 1684007 | 57607192 | 7.7 |
| A387 | 63319317 | 3150093 | 58320095 | 7 |
| A372 | 65150304 | 1747733 | 61780101 | 7.1 |
| A401 | 66448666 | 5160839 | 59727949 | 5.1 |
| A292 | 107849372 | 3084297 | 103172824 | 9.2 |
| A420 | 70359287 | 2239870 | 65611556 | 7.5 |
| A377 | 60296537 | 2949187 | 55918962 | 6.2 |

TABLE 11
ASO

| SEQ | | |
|---|---|---|
| ID | Oligo # | Sequence |
| | FMR1 | |
| NO: 76 | 704 (DNA) | (eA) # (eG) # (eA) # (eA) # (eG) # (eC) # (eC) # (eA) # (eA) # (eA) # (eG) # |
| | | (eG) # (eA) # (eG) # (eA) # (eC) # (eC) # (eT) # (eG) # (eA) |
| NO: 77 | 704 (RNA) | (eA) # (eG) # (eA) # (eA) # (eG) # (eC) # (eC) # (eA) # (eA) # (eA) # (eG) # |
| | | (eG) # (eA) # (eG) # (eA) # (eC) # (eC) # (eU) # (eG) # (eA) |
| NO: 78 | 705 (DNA) | (eA) # (eA) # (eA) # (eG) # (eA) # (eG) # (eA) # (eA) # (eG) # (eC) # (eC) # |
| | | (eA) # (eA) # (eA) # (eG) # (eG) # (eA) # (eG) # (eA) # (eC) |
| NO: 79 | 705 (RNA) | (eA) # (eA) # (eA) # (eG) # (eA) # (eG) # (eA) # (eA) # (eG) # (eC) # (eC) # |
| | | (eA) # (eA) # (eA) # (eG) # (eG) # (eA) # (eG) # (eA) # (eC) |
| NO: 80 | 706 (DNA) | (eC) # (eT) # (eA) # (eG) # (eA) # (eC) # (eC) # (eG) # (eG) # (eA) # (eA) # |
| | | (eA) # (eA) # (eG) # (eA) # (eG) # (eA) # (eA) # (eG) # (eC) # (eC) # (eA) |
| NO: 81 | 706 (RNA) | (eC) # (eU) # (eA) # (eG) # (eA) # (eC) # (eC) # (eG) # (eG) # (eA) # (eA) # |
| | | (eA) # (eA) # (eG) # (eA) # (eG) # (eA) # (eA) # (eG) # (eC) # (eC) # (eA) |
| NO: 82 | 707 (DNA) | (eA) # (eT) # (eG) # (eC) # (eT) # (eA) # (eG) # (eA) # (eC) # (eC) # (eG) # |
| | | (eG) # (eA) # (eA) # (eA) # (eA) # (eG) # (eA) # (eG) # (eA) # (eA) |
| NO: 83 | 707 (RNA) | (eA) # (eU) # (eG) # (eC) # (eU) # (eA) # (eG) # (eA) # (eC) # (eC) # (eG) # |
| | | (eG) # (eA) # (eA) # (eA) # (eA) # (eG) # (eA) # (eG) # (eA) # (eA) |
| NO: 84 | 708 (DNA) | (eC) # (eA) # (eA) # (eT) # (eG) # (eC) # (eT) # (eA) # (eG) # (eA) # (eC) # |
| | | (eC) # (eG) # (eG) # (eA) # (eA) # (eA) # (eA) # (eG) # (eA) |
| NO: 85 | 708 (RNA) | (eC) # (eA) # (eA) # (eU) # (eG) # (eC) # (eU) # (eA) # (eG) # (eA) # (eC) # |
| | | (eC) # (eG) # (eG) # (eA) # (eA) # (eA) # (eA) # (eG) # (eA) |
| NO: 86 | 709 (DNA) | (eA) # (eA) # (eG) # (eT) # (eC) # (eC) # (eC) # (eA) # (eA) # (eT) # (eG) # |
| | | (eC) # (eT) # (eA) # (eG) # (eA) # (eC) # (eC) # (eG) # (eG) # (eA) |
| NO: 87 | 709 (RNA) | (eA) # (eA) # (eG) # (eU) # (eC) # (eC) # (eC) # (eA) # (eA) # (eU) # (eG) # |
| | | (eC) # (eU) # (eA) # (eG) # (eA) # (eC) # (eC) # (eG) # (eG) # (eA) |
| NO: 88 | 710 (DNA) | (eT) # (eC) # (eT) # (eC) # (eC) # (eG) # (eA) # (eA) # (eG) # (eT) # (eC) # |
| | | (eC) # (eC) # (eA) # (eA) # (eT) # (eG) # (eC) # (eT) # (eA) |
| NO: 89 | 710 (RNA) | (eU) # (eC) # (eU) # (eC) # (eC) # (eG) # (eA) # (eA) # (eG) # (eU) # (eC) # |
| | | (eC) # (eC) # (eA) # (eA) # (eU) # (eG) # (eC) # (eU) # (eA) |
| NO: 90 | 711 (DNA) | (eG) # (eA) # (eG) # (eC) # (eT) # (eC) # (eT) # (eC) # (eC) # (eG) # (eA) # |
| | | (eA) # (eG) # (eT) # (eC) # (eC) # (eC) # (eA) |
| NO: 91 | 711 (RNA) | (eG) # (eA) # (eG) # (eC) # (eU) # (eC) # (EU) # (eC) # (eC) # (eG) # (eA) # |
| | | (eA) # (eG) # (eU) # (eC) # (eC) # (eC) # (eA) |
| NO: 92 | 712 (DNA) | (eA) # (eG) # (eA) # (eA) # (eC) # (eA) # (eG) # (eT) # (eG) # (eG) # (eA) # |
| | | (eG) # (eC) # (eT) # (eC) # (eT) # (eC) # (eC) # (eG) # (eA) |
| NO: 93 | 712 (RNA) | (eA) # (eG) # (eA) # (eA) # (eC) # (eA) # (eG) # (eU) # (eG) # (eG) # (eA) # |
| | | (eG) # (eC) # (eU) # (eC) # (eU) # (eC) # (eC) # (eG) # (eA) |
| NO: 94 | 713 (DNA) | (eC) # (eG) # (eC) # (eC) # (eC) # (eA) # (eG) # (eA) # (eA) # (eC) # (eA) # |
| | | (eG) # (eT) # (eG) # (eG) # (eA) # (eG) # (eC) # (eT) # (eC) |
| NO: 95 | 713 (RNA) | (eC) # (eG) # (eC) # (eC) # (eC) # (eA) # (eG) # (eA) # (eA) # (eC) # (eA) # |
| | | (eG) # (eU) # (eG) # (eG) # (eA) # (eG) # (eC) # (eU) # (eC) |
| NO: 96 | 714 (DNA) | (eC) # (eC) # (eT) # (eC) # (eG) # (eC) # (eC) # (eC) # (eA) # (eG) # (eA) # |
| | | (eA) # (eC) # (eA) # (eG) # (eT) # (eG) # (eG) # (eA) # (eG) |
| NO: 97 | 714 (RNA) | (eC) # (eC) # (eU) # (eC) # (eG) # (eC) # (eC) # (eC) # (eA) # (eG) # (eA) # |
| | | (eA) # (eC) # (eA) # (eG) # (eU) # (eG) # (eG) # (eA) # (eG) |
| | MALAT1 | |
| NO: 98 | MALATI | (1A) # (1A) # (1G) # (dC) # (dT) #(dG) # (dC) # (dA) # (dC) # (dT) # (dG) # |
| | (DNA) | (dT) # (dG) # (1C) # (IT) # (1G) |
| NO: 99 | MALATI | (1A) # (1A) #(1G) #(C)#(U) #(G)#(C) #(A) #(C) #(U) # (G) # (U) # (G) # |
| | (RNA) | (1C) # (1U) # (1G) |

Key:
e-2′-O-methoxyethyl (MOE) nucleotide;
l-locked (LNA) nucleotide;
d-deoxy (DNA) nucleotide;
#-phosphorothioate linkage

TABLE 12
Primers

| Gene | Primer name | Sequence (5′-3′) |
|---|---|---|
| FMR1 | Ex1F | TAGCAGGGCTGAAGAGAA |
| | Ex1R | CTTGTAGAAAGCGCCATTG |
| | Ex2R | TTCATGAACATCCTTTACAAATGC |
| | 217R | CAGTGGAGCTCTCCGAAGTC |
| | 217F | TGGAAAAATCACATGTTGGAG |
| GAPDH | GAPDH F | TCCAAAATCAAGTGGGGCGA |
| | GAPDH R | TGATGACCCTTTTGGCTCCC |
| MALAT1 | MALAT1 F | GAAGGAAGGAGCGCTAACGA |
| | MALAT1 R | TACCAACCACTCGCTTTCCC |
| LAIR2 | Lair2 F_h_RT | CCTGGATGGTCTGAGCACA |
| | Lair2 R_h_RT | CATGGTGCATCAAATCCGGA |
| RAB25 | RAB25_h_F | ACTGCTCTTCCTGGAGACCTCA |
| | RAB25_h_R | GCTGTTCTGTCTCTGCTTGGAC |
| AGAP1 | AGAP1_h_F | GAGTGAGGCTACGGTCATTGCA |
| | AGAP1_h_R | TCGGTGCTTCTTTCTGTTGGCG |
| FAM3B | FAM3B_h_F | aatccctgctcttcatggtg |
| | FAM3B_h_R | gagttocaagccttttgotg |
| S100B | S100b_h_F | TGGCCCTCATCGACGTTTTC |
| | S100b_h_R | ATGTTCAAAGAACTCGTGGCA |

TABLE 13
DE_FXSvsTD

| | baseMean | log2FoldChange | lfcSE | stat | pvalue | padj |
|---|---|---|---|---|---|---|
| ANAPC1P2 | 4.18E+01 | 2.46E+01 | 3.17E+00 | 7.75E+00 | 9.19E−15 | 1.70E−10 |
| FAM3B | 5.82E+01 | 5.71E+00 | 1.04E+00 | 5.47E+00 | 4.46E−08 | 2.91E−04 |
| HMGB1P5 | 8.24E+01 | −1.99E+00 | 3.65E−01 | −5.46E+00 | 4.73E−08 | 2.91E−04 |
| CYP4F22 | 1.27E+02 | −1.16E+00 | 2.26E−01 | −5.11E+00 | 3.15E−07 | 1.46E−03 |
| RHOC | 2.69E+03 | −6.80E−01 | 1.35E−01 | −5.04E+00 | 4.69E−07 | 1.56E−03 |
| AGAP1 | 3.43E+02 | −9.92E−01 | 1.97E−01 | −5.02E+00 | 5.07E−07 | 1.56E−03 |
| CFAP70 | 1.41E+02 | 6.52E−01 | 1.41E−01 | 4.61E+00 | 3.97E−06 | 1.04E−02 |
| KNDC1 | 1.35E+02 | −1.33E+00 | 2.92E−01 | −4.57E+00 | 4.82E−06 | 1.04E−02 |
| PRR5L | 1.48E+03 | −5.93E−01 | 1.30E−01 | −4.56E+00 | 5.05E−06 | 1.04E−02 |
| ZNF365 | 1.05E+02 | −1.01E+00 | 2.25E−01 | −4.48E+00 | 7.31E−06 | 1.35E−02 |
| DUSP5 | 7.77E+02 | −6.21E−01 | 1.45E−01 | −4.29E+00 | 1.80E−05 | 2.76E−02 |
| ARHGAP24 | 6.02E+02 | 5.40E−01 | 1.26E−01 | 4.28E+00 | 1.84E−05 | 2.76E−02 |
| EPOP | 3.73E+01 | −9.23E−01 | 2.16E−01 | −4.27E+00 | 1.94E−05 | 2.76E−02 |
| MXRA7 | 9.50E+02 | −1.38E+00 | 3.24E−01 | −4.24E+00 | 2.24E−05 | 2.95E−02 |
| TOMM5 | 1.22E+03 | −2.61E−01 | 6.21E−02 | −4.20E+00 | 2.67E−05 | 3.09E−02 |
| TRBV2 | 1.83E+02 | −7.59E−01 | 1.81E−01 | −4.20E+00 | 2.68E−05 | 3.09E−02 |
| NKG7 | 1.34E+04 | −8.04E−01 | 1.96E−01 | −4.10E+00 | 4.18E−05 | 4.22E−02 |
| CLEC5A | 4.17E+02 | 7.19E−01 | 1.76E−01 | 4.09E+00 | 4.28E−05 | 4.22E−02 |
| TKTL1 | 3.19E+02 | −9.02E−01 | 2.21E−01 | −4.09E+00 | 4.33E−05 | 4.22E−02 |
| RAB25 | 2.16E+01 | 1.21E+00 | 2.97E−01 | 4.07E+00 | 4.69E−05 | 4.34E−02 |
| COL13A1 | 6.11E+01 | −1.39E+00 | 3.44E−01 | −4.04E+00 | 5.35E−05 | 4.71E−02 |
| RBM11 | 8.26E+01 | 6.80E−01 | 1.72E−01 | 3.96E+00 | 7.55E−05 | 6.23E−02 |
| AC008764.4 | 9.35E+00 | 3.06E+00 | 7.77E−01 | 3.94E+00 | 8.20E−05 | 6.23E−02 |
| CKB | 2.31E+02 | −8.70E−01 | 2.21E−01 | −3.93E+00 | 8.33E−05 | 6.23E−02 |
| GNGT2 | 3.68E+02 | −4.43E−01 | 1.13E−01 | −3.93E+00 | 8.43E−05 | 6.23E−02 |
| LAMC3 | 3.36E+01 | 1.19E+00 | 3.03E−01 | 3.91E+00 | 9.19E−05 | 6.40E−02 |
| NEFL | 1.89E+02 | 8.97E−01 | 2.30E−01 | 3.90E+00 | 9.44E−05 | 6.40E−02 |
| ZNF154 | 5.07E+02 | 6.94E−01 | 1.78E−01 | 3.90E+00 | 9.69E−05 | 6.40E−02 |
| C12orf75 | 1.27E+03 | −4.30E−01 | 1.11E−01 | −3.88E+00 | 1.03E−04 | 6.40E−02 |
| MSC-AS1 | 7.91E+01 | −1.20E+00 | 3.09E−01 | −3.88E+00 | 1.05E−04 | 6.40E−02 |
| RPL39L | 7.66E+01 | −8.97E−01 | 2.32E−01 | −3.87E+00 | 1.09E−04 | 6.40E−02 |
| PPFIBP1 | 1.29E+02 | 6.46E−01 | 1.67E−01 | 3.87E+00 | 1.11E−04 | 6.40E−02 |
| ACOT7 | 2.85E+02 | −4.31E−01 | 1.12E−01 | −3.85E+00 | 1.18E−04 | 6.51E−02 |
| CDKN1C | 1.31E+03 | −9.12E−01 | 2.37E−01 | −3.84E+00 | 1.21E−04 | 6.51E−02 |
| CKS1B | 2.92E+02 | −3.35E−01 | 8.72E−02 | −3.84E+00 | 1.25E−04 | 6.51E−02 |
| LINC00174 | 4.19E+02 | 4.50E−01 | 1.18E−01 | 3.83E+00 | 1.28E−04 | 6.51E−02 |
| PALM | 9.57E+00 | −1.60E+00 | 4.17E−01 | −3.83E+00 | 1.30E−04 | 6.51E−02 |
| CABP4 | 5.72E+01 | −8.13E−01 | 2.13E−01 | −3.81E+00 | 1.36E−04 | 6.63E−02 |
| EFNA5 | 2.93E+01 | −1.30E+00 | 3.43E−01 | −3.80E+00 | 1.46E−04 | 6.92E−02 |
| LYPD2 | 1.41E+02 | −1.25E+00 | 3.31E−01 | −3.77E+00 | 1.64E−04 | 7.57E−02 |
| DRAXIN | 1.71E+02 | −1.19E+00 | 3.18E−01 | −3.76E+00 | 1.73E−04 | 7.78E−02 |
| B3GAT1 | 3.80E+02 | −1.29E+00 | 3.45E−01 | −3.73E+00 | 1.93E−04 | 8.17E−02 |
| TPST2 | 4.42E+03 | −3.02E−01 | 8.10E−02 | −3.72E+00 | 1.96E−04 | 8.17E−02 |
| CROCC2 | 2.10E+01 | −1.41E+00 | 3.80E−01 | −3.72E+00 | 1.99E−04 | 8.17E−02 |
| FCRL6 | 1.36E+03 | −7.90E−01 | 2.12E−01 | −3.72E+00 | 2.01E−04 | 8.17E−02 |
| AC026369.3 | 2.30E+01 | −1.28E+00 | 3.45E−01 | −3.71E+00 | 2.03E−04 | 8.17E−02 |
| C19orf12 | 1.19E+03 | −2.70E−01 | 7.32E−02 | −3.69E+00 | 2.24E−04 | 8.72E−02 |
| S100B | 5.77E+02 | −1.95E+00 | 5.28E−01 | −3.69E+00 | 2.26E−04 | 8.72E−02 |
| GAS1 | 4.01E+01 | −1.22E+00 | 3.30E−01 | −3.68E+00 | 2.33E−04 | 8.80E−02 |
| JAKMIP1 | 3.89E+02 | −8.12E−01 | 2.21E−01 | −3.67E+00 | 2.40E−04 | 8.88E−02 |
| LINC02345 | 3.42E+01 | −1.17E+00 | 3.22E−01 | −3.65E+00 | 2.61E−04 | 9.45E−02 |
| GPR153 | 1.42E+02 | −9.19E−01 | 2.53E−01 | −3.63E+00 | 2.81E−04 | 9.86E−02 |
| S1PR5 | 2.89E+03 | −7.25E−01 | 2.00E−01 | −3.63E+00 | 2.87E−04 | 9.86E−02 |
| MIR3150BHG | 2.33E+01 | 1.37E+00 | 3.78E−01 | 3.63E+00 | 2.88E−04 | 9.86E−02 |

TABLE 14
z_score_WBC_FXSvsTD

| | clusters |
|---|---|
| AC008764.4 | 1 |
| ANAPC1P2 | 1 |
| ARHGAP24 | 1 |
| CFAP70 | 1 |
| PPFIBP1 | 1 |
| RBM11 | 1 |
| AC026369.3 | 2 |
| ACOT7 | 2 |
| CABP4 | 2 |
| CDKN1C | 2 |
| CKB | 2 |
| CKS1B | 2 |
| COL13A1 | 2 |
| CYP4F22 | 2 |
| DUSP5 | 2 |
| GNGT2 | 2 |
| KNDC1 | 2 |
| LINC02345 | 2 |
| LYPD2 | 2 |
| MXRA7 | 2 |
| RHOC | 2 |
| TKTL1 | 2 |
| TOMM5 | 2 |
| AGAP1 | 3 |
| B3GAT1 | 3 |
| C12orf75 | 3 |
| C19orf12 | 3 |
| CROCC2 | 3 |
| DRAXIN | 3 |
| EFNA5 | 3 |
| EPOP | 3 |
| FCRL6 | 3 |
| GAS1 | 3 |
| GPR153 | 3 |
| HMGB1P5 | 3 |
| JAKMIP1 | 3 |
| MSC-AS1 | 3 |
| NKG7 | 3 |
| PALM | 3 |
| PRR5L | 3 |
| RPL39L | 3 |
| S100B | 3 |
| S1PR5 | 3 |
| TPST2 | 3 |
| TRBV2 | 3 |
| ZNF365 | 3 |
| CLEC5A | 4 |
| MIR3150BHG | 4 |
| NEFL | 4 |
| ZNF154 | 4 |
| FAM3B | 5 |
| LAMC3 | 5 |
| LINC00174 | 5 |
| RAB25 | 5 |

TABLE 15
SE_WBC_FXSvsTD

| | | | | | | | | | | | | IncLevel |
|---|---|---|---|---|---|---|---|---|---|---|---|---|
| ID | GeneID | chr | strand | exonStart_0base | exonEnd | upstreamES | upstreamEE | downstreamES | downstreamEE | PValue | FDR | Difference |
| 66307 | PARP6 | chr15 | − | 72253949 | 72254070 | 72253713 | 72253762 | 72254454 | 72254481 | 4.13E−05 | 4.77E−03 | −2.610E−01 |
| 71943 | NCALD | chr8 | − | 101887140 | 101887227 | 101719251 | 101719648 | 101915808 | 101915858 | 3.53E−07 | 9.83E−05 | −2.590E−01 |
| 133085 | PACRGL | chr4 | + | 20709682 | 20709773 | 20707802 | 20707870 | 20713431 | 20713521 | 1.40E−11 | 1.94E−08 | −2.560E−01 |
| 30386 | TCF7 | chr5 | + | 134144796 | 134144869 | 134143591 | 134143640 | 134145259 | 134145340 | 3.66E−04 | 2.61E−02 | −2.330E−01 |
| 60067 | ADAM15 | chr1 | + | 155061903 | 155062117 | 155060204 | 155060343 | 155062244 | 155062369 | 5.21E−04 | 3.43E−02 | −2.140E−01 |
| 10686 | LAIR2 | chr19 | + | 54509034 | 54509085 | 54507890 | 54508184 | 54510525 | 54510687 | 3.42E−05 | 4.10E−03 | −2.130E−01 |
| 92498 | XPNPEP3 | chr22 | + | 40860677 | 40860793 | 40857096 | 40857245 | 40861617 | 40862770 | 9.18E−05 | 8.87E−03 | −2.120E−01 |
| 60064 | ADAM15 | chr1 | + | 155061903 | 155061975 | 155060204 | 155060343 | 155062244 | 155062369 | 1.37E−04 | 1.21E−02 | −2.090E−01 |
| 15527 | POLR2J3 | chr7 | − | 102543524 | 102543661 | 102541500 | 102541662 | 102544705 | 102544776 | 1.04E−04 | 9.77E−03 | −2.010E−01 |
| 15530 | POLR2J3 | chr7 | − | 102543524 | 102543697 | 102541500 | 102541662 | 102544705 | 102544776 | 3.32E−04 | 2.42E−02 | −1.910E−01 |
| 34914 | LINC00937 | chr12 | − | 8370212 | 8370429 | 8356962 | 8357141 | 8390269 | 8390969 | 2.73E−04 | 2.05E−02 | −1.890E−01 |
| 45192 | WARS1 | chr14 | − | 100374135 | 100374244 | 100361707 | 100361921 | 100375282 | 100375347 | 1.73E−04 | 1.46E−02 | −1.890E−01 |
| 60059 | ADAM15 | chr1 | + | 155061417 | 155061489 | 155060204 | 155060343 | 155062244 | 155062369 | 3.33E−05 | 4.01E−03 | −1.880E−01 |
| 60055 | ADAM15 | chr1 | + | 155061414 | 155061489 | 155060204 | 155060343 | 155062244 | 155062369 | 3.65E−05 | 4.31E−03 | −1.850E−01 |
| 169309 | AL135818.1 | chr14 | + | 91247489 | 91247659 | 91244354 | 91244499 | 91250764 | 91251228 | 8.11E−07 | 1.99E−04 | −1.820E−01 |
| 10872 | AC092070.2 | chr19 | + | 53200623 | 53200703 | 53197110 | 53197262 | 53204015 | 53204055 | 5.18E−06 | 8.73E−04 | −1.800E−01 |
| 147682 | TRPT1 | chr11 | − | 64224099 | 64224162 | 64223829 | 64223967 | 64224284 | 64224341 | 1.71E−04 | 1.45E−02 | −1.800E−01 |
| 22908 | DST | chr6 | − | 56468981 | 56468999 | 56466077 | 56466195 | 56469882 | 56469957 | 5.43E−05 | 5.85E−03 | −1.770E−01 |
| 45198 | WARS1 | chr14 | − | 100374135 | 100374265 | 100361707 | 100361921 | 100375282 | 100375333 | 4.29E−05 | 4.89E−03 | −1.770E−01 |
| 103430 | MIR4435-2HG | chr2 | − | 111239801 | 111239996 | 111233627 | 111233739 | 111248147 | 111248241 | 2.76E−08 | 1.26E−05 | −1.770E−01 |
| 60001 | LRRFIP1 | chr2 | + | 237739231 | 237739309 | 237720771 | 237720822 | 237748363 | 237748399 | 1.25E−04 | 1.13E−02 | −1.670E−01 |
| 90835 | ADCY10P1 | chr6 | + | 41132874 | 41133000 | 41132293 | 41132513 | 41134556 | 41134729 | 4.03E−04 | 2.82E−02 | −1.610E−01 |
| 122318 | RNF19A | chr8 | − | 100300527 | 100300651 | 100287500 | 100288267 | 100303159 | 100303259 | 3.69E−04 | 2.62E−02 | −1.590E−01 |
| 85805 | DRAM2 | chr1 | − | 111139587 | 111139711 | 111137522 | 111137586 | 111140037 | 111140062 | 4.04E−05 | 4.68E−03 | −1.570E−01 |
| 50825 | TRPV2 | chr17 | + | 16433573 | 16433698 | 16428816 | 16428982 | 16436788 | 16437003 | 2.05E−04 | 1.65E−02 | −1.560E−01 |
| 32226 | CAST | chr5 | + | 96740744 | 96740783 | 96740037 | 96740118 | 96741265 | 96741317 | 5.98E−11 | 6.79E−08 | −1.540E−01 |
| 52670 | C11orf80 | chr11 | + | 66756352 | 66756505 | 66748378 | 66748517 | 66759042 | 66759084 | 3.21E−04 | 2.36E−02 | −1.540E−01 |
| 192183 | ZNF266 | chr19 | − | 9433667 | 9433786 | 9420064 | 9420218 | 9434797 | 9434859 | 4.78E−05 | 5.35E−03 | −1.540E−01 |
| 59607 | HMOX2 | chr16 | + | 4483636 | 4483738 | 4476411 | 4476487 | 4496145 | 4496762 | 1.82E−05 | 2.48E−03 | −1.520E−01 |
| 175479 | JPX | chrX | + | 73997040 | 73997166 | 73946998 | 73947374 | 73998767 | 73998844 | 2.91E−13 | 6.92E−10 | −1.520E−01 |
| 192179 | ZNF266 | chr19 | − | 9433667 | 9433783 | 9420064 | 9420218 | 9434797 | 9434859 | 1.03E−04 | 9.68E−03 | −1.500E−01 |
| 7453 | DPM1 | chr20 | − | 50941128 | 50941209 | 50940864 | 50940933 | 50945736 | 50945762 | 4.29E−08 | 1.78E−05 | −1.440E−01 |
| 36444 | FRG1 | chr4 | + | 189955036 | 189955151 | 189952161 | 189952287 | 189957397 | 189957502 | 9.86E−05 | 9.35E−03 | −1.410E−01 |
| 36443 | FRG1 | chr4 | + | 189953067 | 189953125 | 189952161 | 189952287 | 189957397 | 189957502 | 4.98E−04 | 3.31E−02 | −1.400E−01 |
| 183737 | COPS3 | chr17 | − | 17261599 | 17261692 | 17254886 | 17254945 | 17261965 | 17262106 | 4.89E−05 | 5.44E−03 | −1.360E−01 |
| 22304 | METTL8 | chr2 | − | 171330558 | 171330616 | 171326041 | 171326148 | 171337452 | 171337502 | 1.60E−08 | 7.78E−06 | −1.350E−01 |
| 11473 | FCRLA | chr1 | + | 161710474 | 161710492 | 161706971 | 161707343 | 161710759 | 161710912 | 4.32E−04 | 2.96E−02 | −1.340E−01 |
| 45197 | WARS1 | chr14 | − | 100374135 | 100374244 | 100369086 | 100369258 | 100376259 | 100376334 | 6.02E−09 | 3.43E−06 | −1.340E−01 |
| 45204 | WARS1 | chr14 | − | 100374135 | 100374265 | 100369086 | 100369258 | 100376259 | 100376285 | 3.15E−09 | 1.91E−06 | −1.310E−01 |
| 69313 | PMS2CL | chr7 | + | 6733398 | 6733527 | 6731896 | 6731955 | 6735304 | 6735389 | 7.25E−04 | 4.45E−02 | −1.280E−01 |
| 82169 | MUC20-OT1 | chr3 | + | 195663623 | 195663767 | 195662570 | 195662733 | 195664085 | 195664250 | 5.26E−05 | 5.72E−03 | −1.280E−01 |
| 38851 | ZNF273 | chr7 | + | 64918196 | 64918292 | 64903246 | 64903419 | 64927653 | 64929124 | 1.51E−07 | 5.06E−05 | −1.270E−01 |
| 40300 | NQO2 | chr6 | + | 3003668 | 3003789 | 3002049 | 3002286 | 3006467 | 3006559 | 1.12E−05 | 1.65E−03 | −1.270E−01 |
| 120894 | AC141586.1 | chr16 | + | 2613959 | 2614068 | 2603419 | 2603727 | 2628824 | 2630494 | 3.03E−07 | 8.65E−05 | −1.260E−01 |
| 157606 | PNPO | chr17 | + | 47945558 | 47945612 | 47944615 | 47944715 | 47946322 | 47946393 | 1.85E−05 | 2.51E−03 | −1.250E−01 |
| 31474 | BFAR | chr16 | + | 14649803 | 14649973 | 14644287 | 14644609 | 14661891 | 14662065 | 2.33E−07 | 7.10E−05 | −1.200E−01 |
| 103134 | GBAP1 | chr1 | − | 155218387 | 155218479 | 155217239 | 155218049 | 155218847 | 155218943 | 1.00E−06 | 2.35E−04 | −1.190E−01 |
| 135245 | RAB18 | chr10 | + | 27531528 | 27531615 | 27509874 | 27509930 | 27532506 | 27532579 | 1.38E−06 | 3.02E−04 | −1.150E−01 |
| 52933 | GPS1 | chr17 | + | 82052255 | 82052459 | 82051499 | 82051524 | 82053273 | 82053366 | 3.45E−04 | 2.48E−02 | −1.140E−01 |
| 190346 | TAF5 | chr10 | + | 103378234 | 103378550 | 103373357 | 103373595 | 103379607 | 103379771 | 2.32E−05 | 2.99E−03 | −1.100E−01 |
| 59611 | HMOX2 | chr16 | + | 4483636 | 4483754 | 4476339 | 4476487 | 4496145 | 4496762 | 3.00E−05 | 3.69E−03 | −1.090E−01 |
| 133087 | PACRGL | chr4 | + | 20712787 | 20712922 | 20707802 | 20707870 | 20713431 | 20713516 | 7.13E−12 | 1.09E−08 | −1.090E−01 |
| 156330 | IZUMO4 | chr19 | + | 2097928 | 2098127 | 2097251 | 2097495 | 2098286 | 2098334 | 6.33E−10 | 4.73E−07 | −1.090E−01 |
| 37101 | ANKS3 | chr16 | − | 4726978 | 4727177 | 4724749 | 4724831 | 4729979 | 4730151 | 1.48E−06 | 3.18E−04 | −1.060E−01 |
| 45019 | SEPTIN2 | chr2 | + | 241316421 | 241316540 | 241315881 | 241315982 | 241325992 | 241326113 | 3.83E−05 | 4.48E−03 | −1.060E−01 |
| 45390 | YY1AP1 | chr1 | − | 155680381 | 155680456 | 155679408 | 155679512 | 155688070 | 155688201 | 3.89E−07 | 1.06E−04 | −1.060E−01 |
| 2595 | OFD1 | chrX | + | 13752703 | 13753441 | 13751248 | 13751368 | 13756577 | 13756767 | 9.82E−06 | 1.48E−03 | −1.040E−01 |
| 181022 | AC012184.3 | chr16 | − | 70341939 | 70342099 | 70333782 | 70333960 | 70346198 | 70346747 | 1.57E−05 | 2.18E−03 | −1.020E−01 |
| 45028 | SEPTIN2 | chr2 | + | 241316421 | 241316575 | 241315958 | 241315982 | 241325992 | 241326113 | 3.17E−04 | 2.33E−02 | −1.000E−01 |
| 10684 | LAIR2 | chr19 | + | 54507890 | 54508184 | 54503699 | 54503735 | 54510525 | 54510687 | 8.07E−12 | 1.20E−08 | −9.900E−02 |
| 45025 | SEPTIN2 | chr2 | + | 241316421 | 241316555 | 241315860 | 241315982 | 241325992 | 241326113 | 5.23E−04 | 3.43E−02 | −9.900E−02 |
| 47519 | LDAH | chr2 | − | 20739970 | 20740205 | 20701569 | 20701652 | 20774809 | 20774979 | 4.09E−05 | 4.72E−03 | −9.900E−02 |
| 147539 | CDC42BPG | chr11 | − | 64827685 | 64827783 | 64827526 | 64827611 | 64829470 | 64830070 | 3.34E−11 | 4.14E−08 | −9.900E−02 |
| 38854 | ZNF273 | chr7 | + | 64918196 | 64918292 | 64917580 | 64917707 | 64927653 | 64929124 | 1.40E−07 | 4.80E−05 | −9.800E−02 |
| 98945 | VMP1 | chr17 | + | 59834011 | 59834050 | 59811669 | 59811786 | 59838294 | 59838397 | 4.68E−04 | 3.15E−02 | −9.600E−02 |
| 104662 | ATP6AP1L | chr5 | + | 82304933 | 82305481 | 82304614 | 82304850 | 82310156 | 82310207 | 4.63E−04 | 3.13E−02 | −9.600E−02 |
| 115640 | COMMD2 | chr3 | − | 149750256 | 149750382 | 149741389 | 149741718 | 149751402 | 149751485 | 6.09E−04 | 3.88E−02 | −9.500E−02 |
| 179439 | PPRC1 | chr10 | + | 102138878 | 102138980 | 102137849 | 102138038 | 102139099 | 102142004 | 6.73E−05 | 6.94E−03 | −9.500E−02 |
| 188419 | RHBDF2 | chr17 | − | 76487973 | 76488132 | 76487711 | 76487851 | 76501352 | 76501412 | 8.86E−06 | 1.36E−03 | −9.500E−02 |
| 193979 | ZNF56 | chr19 | + | 19786608 | 19786691 | 19776635 | 19776994 | 19806280 | 19806376 | 4.90E−04 | 3.27E−02 | −9.500E−02 |
| 82035 | SRSF4 | chr1 | − | 29166728 | 29166912 | 29160374 | 29160517 | 29168502 | 29168638 | 9.13E−12 | 1.33E−08 | −9.400E−02 |
| 171328 | ZNF529-AS1 | chr19 | + | 36594155 | 36594299 | 36593845 | 36593931 | 36594441 | 36594694 | 4.85E−05 | 5.40E−03 | −9.200E−02 |
| 179058 | TNK2 | chr3 | − | 195882444 | 195882527 | 195879082 | 195879175 | 195883156 | 195883309 | 4.67E−04 | 3.15E−02 | −9.200E−02 |
| 37095 | ANKS3 | chr16 | − | 4726658 | 4726776 | 4724791 | 4724831 | 4729979 | 4730151 | 1.18E−06 | 2.67E−04 | −9.100E−02 |
| 56298 | SUCO | chr1 | + | 172585857 | 172585948 | 172579201 | 172579267 | 172588759 | 172588924 | 8.87E−05 | 8.67E−03 | −9.000E−02 |
| 44007 | TBC1D19 | chr4 | + | 26717932 | 26718017 | 26688344 | 26688407 | 26720080 | 26720125 | 2.91E−08 | 1.31E−05 | −8.900E−02 |
| 45189 | WARS1 | chr14 | − | 100374135 | 100374179 | 100369086 | 100369258 | 100376259 | 100376805 | 3.29E−10 | 2.78E−07 | −8.900E−02 |
| 157316 | ITGB7 | chr12 | − | 53201146 | 53201194 | 53200242 | 53200446 | 53207201 | 53207281 | 8.02E−06 | 1.25E−03 | −8.900E−02 |
| 82542 | CYB5RL | chr1 | − | 54190747 | 54190896 | 54187651 | 54187739 | 54195418 | 54195616 | 1.00E−04 | 9.49E−03 | −8.800E−02 |
| 45195 | WARS1 | chr14 | − | 100374135 | 100374244 | 100369086 | 100369258 | 100375282 | 100375347 | 9.70E−12 | 1.40E−08 | −8.600E−02 |
| 58349 | IFI44L | chr1 | + | 78627905 | 78628393 | 78620877 | 78620971 | 78628950 | 78628999 | 2.03E−05 | 2.69E−03 | −8.600E−02 |
| 15102 | TGIF1 | chr18 | + | 3450321 | 3450505 | 3449536 | 3449656 | 3456353 | 3456580 | 7.31E−05 | 7.44E−03 | −8.500E−02 |
| 69818 | ZBTB25 | chr14 | − | 64499440 | 64499563 | 64490360 | 64490540 | 64504486 | 64504584 | 2.44E−06 | 4.78E−04 | −8.400E−02 |
| 54030 | FKRP | chr19 | + | 46748514 | 46748621 | 46748026 | 46748088 | 46755411 | 46755768 | 1.92E−04 | 1.58E−02 | −8.300E−02 |
| 16566 | NSUN5P1 | chr7 | + | 75414639 | 75414798 | 75412788 | 75412932 | 75414884 | 75415024 | 8.05E−04 | 4.84E−02 | −8.200E−02 |
| 60050 | ADAM15 | chr1 | + | 155060762 | 155060832 | 155060204 | 155060343 | 155062244 | 155062369 | 4.27E−07 | 1.15E−04 | −8.200E−02 |
| 134222 | PRKCQ-AS1 | chr10 | + | 6615369 | 6615637 | 6596845 | 6596894 | 6615762 | 6615893 | 1.31E−12 | 2.48E−09 | −8.200E−02 |
| 147683 | TRPT1 | chr11 | − | 64224099 | 64224210 | 64223798 | 64223967 | 64224284 | 64224341 | 3.63E−11 | 4.46E−08 | −8.200E−02 |
| 13796 | NCAPG2 | chr7 | − | 158650831 | 158650972 | 158646459 | 158646563 | 158652292 | 158652480 | 4.67E−05 | 5.23E−03 | −8.100E−02 |
| 80920 | IP6K2 | chr3 | − | 48694458 | 48694525 | 48693452 | 48694240 | 48695089 | 48695421 | 3.17E−04 | 2.34E−02 | −8.100E−02 |
| 91691 | ALS2CL | chr3 | − | 46683129 | 46683326 | 46682028 | 46682094 | 46683781 | 46683848 | 5.57E−06 | 9.27E−04 | −8.100E−02 |
| 8575 | NFS1 | chr20 | − | 35675044 | 35675202 | 35674511 | 35674617 | 35680736 | 35680871 | 3.47E−05 | 4.14E−03 | −8.000E−02 |
| 59872 | LINC00174 | chr7 | − | 66476258 | 66476327 | 66450070 | 66450236 | 66479008 | 66479096 | 1.41E−06 | 3.07E−04 | −8.000E−02 |
| 43756 | CYRIB | chr8 | − | 129948965 | 129949067 | 129912469 | 129912515 | 129970942 | 129970995 | 1.23E−07 | 4.28E−05 | −7.900E−02 |
| 50858 | CDC27 | chr17 | − | 47171916 | 47172064 | 47169916 | 47170042 | 47181561 | 47181637 | 7.60E−04 | 4.62E−02 | −7.900E−02 |
| 174873 | ZNF202 | chr11 | − | 123727475 | 123727595 | 123723913 | 123726991 | 123728132 | 123728262 | 1.32E−04 | 1.17E−02 | −7.900E−02 |
| 184886 | GOLGA2P5 | chr12 | − | 100168451 | 100168633 | 100167549 | 100167639 | 100168923 | 100169199 | 1.97E−08 | 9.30E−06 | −7.900E−02 |
| 194100 | ZNF85 | chr19 | + | 20944251 | 20944296 | 20934969 | 20935047 | 20948743 | 20949169 | 1.86E−04 | 1.54E−02 | −7.900E−02 |
| 54579 | FBXW8 | chr12 | + | 116985205 | 116985402 | 116964696 | 116964854 | 116988662 | 116988869 | 1.98E−10 | 1.81E−07 | −7.800E−02 |
| 127651 | COA8 | chr14 | + | 103571622 | 103571820 | 103562961 | 103563124 | 103587273 | 103587364 | 4.51E−04 | 3.07E−02 | −7.800E−02 |
| 184546 | NSRP1 | chr17 | + | 30163066 | 30163162 | 30117301 | 30117332 | 30172541 | 30172598 | 7.97E−05 | 7.95E−03 | −7.800E−02 |
| 154699 | KLRC4-KLRK1 | chr12 | − | 10391834 | 10391891 | 10389942 | 10390027 | 10405849 | 10405950 | 7.17E−05 | 7.32E−03 | −7.700E−02 |
| 2599 | OFD1 | chrX | + | 13753367 | 13753441 | 13751248 | 13751368 | 13756577 | 13756767 | 3.23E−06 | 5.97E−04 | −7.500E−02 |
| 54035 | FKRP | chr19 | + | 46748514 | 46748884 | 46748026 | 46748088 | 46755411 | 46755449 | 1.12E−04 | 1.03E−02 | −7.500E−02 |
| 137500 | BCLAF3 | chrX | − | 19929784 | 19929940 | 19912859 | 19917334 | 19937504 | 19937532 | 6.04E−04 | 3.85E−02 | −7.500E−02 |
| 15359 | SRPK2 | chr7 | − | 105268805 | 105268869 | 105203627 | 105203785 | 105297420 | 105297533 | 1.22E−06 | 2.73E−04 | −7.300E−02 |
| 45202 | WARS1 | chr14 | − | 100374135 | 100374265 | 100369086 | 100369258 | 100375282 | 100375333 | 1.03E−13 | 2.84E−10 | −7.300E−02 |
| 48027 | HPCAL1 | chr2 | + | 10426219 | 10426495 | 10424524 | 10424613 | 10426723 | 10427352 | 1.27E−06 | 2.83E−04 | −7.300E−02 |
| 126021 | RXYLT1 | chr12 | + | 63782540 | 63782704 | 63781018 | 63781174 | 63784969 | 63785072 | 6.99E−04 | 4.32E−02 | −7.300E−02 |
| 25875 | CARMIL1 | chr6 | + | 25604811 | 25604893 | 25600313 | 25600746 | 25606060 | 25606273 | 2.40E−04 | 1.85E−02 | −7.200E−02 |
| 187107 | RAD51C | chr17 | + | 58705877 | 58706022 | 58703195 | 58703329 | 58709858 | 58709990 | 9.33E−05 | 8.97E−03 | −7.200E−02 |
| 187232 | HEATR6 | chr17 | − | 60046024 | 60046099 | 60043672 | 60044134 | 60047308 | 60047405 | 3.33E−07 | 9.41E−05 | −7.200E−02 |
| 45641 | CROCC | chr1 | + | 16950952 | 16951122 | 16946760 | 16946991 | 16953301 | 16953481 | 1.45E−08 | 7.20E−06 | −7.100E−02 |
| 55081 | AL732372.2 | chr1 | − | 502861 | 502955 | 501587 | 501620 | 514358 | 514423 | 4.19E−06 | 7.33E−04 | −7.100E−02 |
| 142312 | DTNB | chr2 | − | 25607235 | 25607321 | 25596085 | 25596240 | 25628170 | 25628384 | 1.51E−05 | 2.11E−03 | −7.100E−02 |
| 34175 | PLPP1 | chr5 | − | 55475298 | 55475450 | 55467868 | 55468149 | 55490959 | 55491114 | 2.31E−05 | 2.98E−03 | −7.000E−02 |
| 58394 | CTNS | chr17 | + | 3647443 | 3647522 | 3640146 | 3640267 | 3654997 | 3655101 | 2.82E−06 | 5.37E−04 | −7.000E−02 |
| 84986 | COP1 | chr1 | − | 176175909 | 176176007 | 176149005 | 176149074 | 176184632 | 176184664 | 6.87E−05 | 7.06E−03 | −7.000E−02 |
| 168274 | NEK3 | chr13 | − | 52135728 | 52135863 | 52133688 | 52133815 | 52136115 | 52136232 | 2.07E−05 | 2.74E−03 | −7.000E−02 |
| 2196 | POLA1 | chrX | + | 24745417 | 24745542 | 24743229 | 24743329 | 24748310 | 24748460 | 4.14E−04 | 2.87E−02 | −6.800E−02 |
| 21109 | LSM14B | chr20 | + | 62126225 | 62126439 | 62124616 | 62124780 | 62130218 | 62130296 | 1.51E−04 | 1.30E−02 | −6.800E−02 |
| 85585 | CCDC18-AS1 | chr1 | − | 93269682 | 93269758 | 93264637 | 93264772 | 93318167 | 93318228 | 1.83E−04 | 1.51E−02 | −6.800E−02 |
| 113790 | LYPLAL1-DT | chr1 | − | 219156119 | 219156199 | 219149938 | 219150031 | 219173469 | 219173493 | 5.77E−04 | 3.71E−02 | −6.800E−02 |
| 826 | SLC25A43 | chrX | + | 119410189 | 119410362 | 119399408 | 119399678 | 119452008 | 119452143 | 1.91E−06 | 3.93E−04 | −6.700E−02 |
| 137231 | BRAF | chr7 | − | 140808236 | 140808316 | 140807959 | 140808062 | 140808891 | 140808995 | 2.75E−05 | 3.42E−03 | −6.700E−02 |
| 146655 | STX3 | chr11 | + | 59795601 | 59795712 | 59793379 | 59793514 | 59797282 | 59797396 | 5.00E−05 | 5.52E−03 | −6.700E−02 |
| 149082 | PPFIA1 | chr11 | + | 70350993 | 70351023 | 70348188 | 70348420 | 70354300 | 70354452 | 2.51E−05 | 3.18E−03 | −6.700E−02 |
| 23540 | UBR2 | chr6 | + | 42592150 | 42592229 | 42573733 | 42573993 | 42603587 | 42603718 | 9.35E−05 | 8.98E−03 | −6.600E−02 |
| 27934 | SRP14-AS1 | chr15 | + | 40060096 | 40060263 | 40045960 | 40046069 | 40065220 | 40065705 | 1.43E−04 | 1.25E−02 | −6.500E−02 |
| 74506 | ZBTB7B | chr1 | + | 155010915 | 155011015 | 155010257 | 155010397 | 155014014 | 155014093 | 4.05E−10 | 3.31E−07 | −6.500E−02 |
| 137506 | BCLAF3 | chrX | − | 19935808 | 19935898 | 19912859 | 19917334 | 19937504 | 19937532 | 2.04E−04 | 1.64E−02 | −6.400E−02 |
| 4130 | SFI1 | chr22 | + | 31603743 | 31603819 | 31602606 | 31602785 | 31604308 | 31604404 | 8.00E−12 | 1.20E−08 | −6.300E−02 |
| 54033 | FKRP | chr19 | + | 46748514 | 46748717 | 46748026 | 46748088 | 46755411 | 46755610 | 1.96E−04 | 1.60E−02 | −6.300E−02 |
| 139762 | STAG3L3 | chr7 | − | 72999054 | 72999162 | 72979943 | 72980017 | 72999470 | 72999550 | 1.40E−05 | 1.99E−03 | −6.300E−02 |
| 144901 | IMMP1L | chr11 | − | 31473723 | 31473786 | 31463171 | 31463305 | 31509518 | 31509594 | 2.67E−11 | 3.43E−08 | −6.300E−02 |
| 157609 | PNPO | chr17 | + | 47945860 | 47945989 | 47944615 | 47944715 | 47946322 | 47946393 | 3.41E−06 | 6.23E−04 | −6.300E−02 |
| 180000 | BBS2 | chr16 | − | 56500853 | 56500977 | 56499777 | 56499907 | 56501352 | 56501594 | 5.46E−05 | 5.88E−03 | −6.300E−02 |
| 7954 | PIGT | chr20 | + | 45418771 | 45418979 | 45416516 | 45416694 | 45419294 | 45419395 | 6.18E−04 | 3.92E−02 | −6.200E−02 |
| 32878 | PAAF1 | chr11 | + | 73878782 | 73878819 | 73876996 | 73877068 | 73887353 | 73887457 | 1.82E−06 | 3.77E−04 | −6.200E−02 |
| 40297 | NQO2 | chr6 | + | 3003668 | 3003789 | 2999875 | 3000085 | 3006467 | 3006559 | 5.29E−10 | 4.15E−07 | −6.200E−02 |
| 116490 | NT5DC2 | chr3 | − | 52524816 | 52524881 | 52524384 | 52524611 | 52524962 | 52525103 | 1.97E−05 | 2.63E−03 | −6.200E−02 |
| 134213 | PRKCQ-AS1 | chr10 | + | 6608347 | 6608408 | 6596845 | 6596894 | 6615762 | 6615909 | 1.22E−12 | 2.38E−09 | −6.200E−02 |
| 142875 | OSBPL5 | chr11 | − | 3120543 | 3120624 | 3119546 | 3119631 | 3121996 | 3122098 | 3.84E−04 | 2.71E−02 | −6.200E−02 |
| 160923 | MAPKAPK5 | chr12 | + | 111868752 | 111868861 | 111866155 | 111866231 | 111871084 | 111871180 | 1.45E−05 | 2.04E−03 | −6.200E−02 |
| 182422 | ITGAE | chr17 | − | 3723287 | 3723383 | 3716687 | 3716798 | 3723687 | 3723744 | 1.81E−07 | 5.85E−05 | −6.200E−02 |
| 193215 | PCBP1-AS1 | chr2 | − | 70081369 | 70081490 | 70051202 | 70051305 | 70083537 | 70083687 | 7.56E−06 | 1.19E−03 | −6.200E−02 |
| 21383 | FAM229B | chr6 | + | 112097040 | 112097201 | 112087590 | 112087720 | 112099269 | 112099408 | 1.81E−05 | 2.47E−03 | −6.100E−02 |
| 53646 | ZSCAN25 | chr7 | + | 99621372 | 99621574 | 99619549 | 99619993 | 99622548 | 99622640 | 9.90E−06 | 1.49E−03 | −6.100E−02 |
| 68430 | OBSCN | chr1 | + | 228299881 | 228300145 | 228298453 | 228298717 | 228303674 | 228303938 | 2.61E−05 | 3.30E−03 | −6.100E−02 |
| 120683 | HDAC10 | chr22 | − | 50247691 | 50247776 | 50246874 | 50246966 | 50247889 | 50247948 | 1.96E−05 | 2.62E−03 | −6.100E−02 |
| 160867 | ACAD10 | chr12 | + | 111727961 | 111728143 | 111721670 | 111721739 | 111733922 | 111734047 | 7.99E−08 | 2.98E−05 | −6.100E−02 |
| 175470 | JPX | chrX | + | 73994839 | 73994961 | 73946998 | 73947374 | 73998767 | 73998844 | 2.10E−11 | 2.78E−08 | −6.100E−02 |
| 176726 | NUBP2 | chr16 | + | 1786756 | 1786955 | 1786536 | 1786655 | 1787676 | 1787777 | 7.35E−04 | 4.50E−02 | −6.100E−02 |
| 9016 | FRG1BP | chr20 | + | 30391196 | 30391308 | 30388452 | 30388578 | 30393550 | 30393655 | 4.31E−04 | 2.96E−02 | −6.000E−02 |
| 18576 | PDPR | chr16 | + | 70130422 | 70130544 | 70128783 | 70129122 | 70131301 | 70131419 | 7.53E−04 | 4.58E−02 | −6.000E−02 |
| 115021 | RAB3IP | chr12 | + | 69800208 | 69800332 | 69795140 | 69795344 | 69812777 | 69813033 | 2.98E−10 | 2.61E−07 | −6.000E−02 |
| 142717 | HEATR3 | chr16 | + | 50066366 | 50066539 | 50065969 | 50066269 | 50068779 | 50068867 | 4.11E−07 | 1.11E−04 | −6.000E−02 |
| 21108 | LSM14B | chr20 | + | 62126225 | 62126439 | 62124616 | 62124780 | 62129784 | 62129952 | 2.59E−05 | 3.28E−03 | −5.800E−02 |
| 70253 | POGLUT3 | chr11 | − | 108482005 | 108482222 | 108477606 | 108477711 | 108486156 | 108486440 | 2.48E−05 | 3.15E−03 | −5.800E−02 |
| 76547 | TPP2 | chr13 | + | 102651338 | 102651397 | 102648992 | 102649151 | 102657055 | 102657207 | 2.08E−07 | 6.48E−05 | −5.800E−02 |
| 127019 | TRPM7 | chr15 | − | 50578558 | 50578664 | 50575868 | 50575919 | 50580873 | 50580908 | 2.19E−05 | 2.88E−03 | −5.800E−02 |
| 37871 | NDUFAF5 | chr20 | + | 13794837 | 13794941 | 13793159 | 13793227 | 13798460 | 13798500 | 1.36E−04 | 1.20E−02 | −5.700E−02 |
| 45191 | WARS1 | chr14 | − | 100374135 | 100374220 | 100369086 | 100369258 | 100375282 | 100375344 | 1.07E−10 | 1.08E−07 | −5.700E−02 |
| 115024 | RAB3IP | chr12 | + | 69800208 | 69800337 | 69795140 | 69795344 | 69812777 | 69812834 | 3.88E−10 | 3.20E−07 | −5.700E−02 |
| 145680 | LARGE2 | chr11 | + | 45928176 | 45928372 | 45927748 | 45928069 | 45928629 | 45929082 | 1.94E−06 | 3.97E−04 | −5.700E−02 |
| 152053 | DPAGT1 | chr11 | − | 119097854 | 119097925 | 119097463 | 119097551 | 119098402 | 119098487 | 5.82E−04 | 3.74E−02 | −5.700E−02 |
| 154960 | DUSP16 | chr12 | − | 12500518 | 12500682 | 12487027 | 12487187 | 12519861 | 12520000 | 8.11E−08 | 3.01E−05 | −5.700E−02 |
| 19295 | KDM3B | chr5 | + | 138386288 | 138386621 | 138381515 | 138381590 | 138391012 | 138392261 | 1.42E−05 | 2.01E−03 | −5.600E−02 |
| 33333 | KMT2B | chr19 | + | 35719468 | 35719541 | 35717972 | 35718381 | 35719783 | 35721804 | 3.80E−06 | 6.75E−04 | −5.600E−02 |
| 40304 | NQO2 | chr6 | + | 3004494 | 3004651 | 3002049 | 3002286 | 3006467 | 3006559 | 3.73E−07 | 1.03E−04 | −5.600E−02 |
| 108026 | COA1 | chr7 | − | 43650611 | 43650712 | 43647227 | 43648652 | 43656032 | 43656153 | 1.57E−14 | 5.19E−11 | −5.600E−02 |
| 41209 | GARS1-DT | chr7 | − | 30551317 | 30551395 | 30550567 | 30550781 | 30563722 | 30564617 | 5.76E−06 | 9.50E−04 | −5.500E−02 |
| 76550 | TPP2 | chr13 | + | 102651358 | 102651397 | 102648906 | 102649151 | 102657055 | 102657207 | 3.99E−04 | 2.80E−02 | −5.500E−02 |
| 80188 | NMRK1 | chr9 | − | 75069741 | 75069813 | 75068995 | 75069102 | 75069894 | 75070042 | ######## | ####### | −5.500E−02 |
| 95143 | MMEL1 | chr1 | − | 2591931 | 2592027 | 2591556 | 2591633 | 2592654 | 2592720 | 8.06E−06 | 1.26E−03 | −5.500E−02 |
| 101319 | ABCA11P | chr4 | − | 437445 | 437511 | 425814 | 426973 | 472574 | 472701 | 2.22E−04 | 1.74E−02 | −5.500E−02 |
| 127462 | UPP1 | chr7 | + | 48103296 | 48103411 | 48101823 | 48101870 | 48103781 | 48103991 | 2.29E−07 | 7.03E−05 | −5.500E−02 |
| 146633 | PHF1 | chr6 | + | 33414954 | 33415081 | 33414724 | 33414829 | 33415260 | 33415329 | 4.31E−06 | 7.51E−04 | −5.500E−02 |
| 152735 | TMEM218 | chr11 | − | 125110643 | 125110746 | 125102131 | 125102809 | 125111538 | 125111579 | 3.02E−05 | 3.70E−03 | −5.500E−02 |
| 533 | MAP7D3 | chrX | − | 136231543 | 136232097 | 136230838 | 136230966 | 136236243 | 136236339 | 3.35E−07 | 9.45E−05 | −5.400E−02 |
| 135613 | ZNF653 | chr19 | − | 11500677 | 11500815 | 11498295 | 11498339 | 11505487 | 11505500 | 4.56E−04 | 3.09E−02 | −5.400E−02 |
| 154409 | AC010175.1 | chr12 | + | 9247580 | 9247677 | 9246468 | 9246548 | 9255558 | 9255692 | 3.58E−04 | 2.56E−02 | −5.400E−02 |
| 181196 | DHODH | chr16 | + | 72022361 | 72022475 | 72017023 | 72017106 | 72023164 | 72023318 | 8.17E−07 | 2.00E−04 | −5.400E−02 |
| 183453 | TUBGCP6 | chr22 | − | 50220191 | 50221874 | 50219956 | 50220015 | 50222027 | 50222102 | 6.63E−05 | 6.86E−03 | −5.400E−02 |
| 183455 | TUBGCP6 | chr22 | − | 50221274 | 50221874 | 50219956 | 50220015 | 50222027 | 50222102 | 1.22E−04 | 1.11E−02 | −5.400E−02 |
| 67041 | TTLL3 | chr3 | + | 9817644 | 9817759 | 9813247 | 9813345 | 9826996 | 9827072 | 4.18E−06 | 7.33E−04 | −5.300E−02 |
| 96700 | TEX10 | chr9 | − | 100308499 | 100308681 | 100303631 | 100303842 | 100320264 | 100320398 | 2.70E−07 | 7.88E−05 | −5.300E−02 |
| 156810 | SPATS2 | chr12 | + | 49486249 | 49486348 | 49484589 | 49484669 | 49489464 | 49489573 | 1.23E−04 | 1.12E−02 | −5.300E−02 |
| 24123 | ZNF76 | chr6 | + | 35293750 | 35293915 | 35292880 | 35293044 | 35294455 | 35294552 | 8.27E−04 | 4.95E−02 | −5.200E−02 |
| 54031 | FKRP | chr19 | + | 46748514 | 46748665 | 46747658 | 46748088 | 46755411 | 46755463 | 6.12E−06 | 9.99E−04 | −5.200E−02 |
| 99946 | SAR1B | chr5 | − | 134632076 | 134632195 | 134623961 | 134624037 | 134632727 | 134632774 | 3.18E−05 | 3.85E−03 | −5.200E−02 |
| 102530 | ZEB2 | chr2 | − | 144398300 | 144400270 | 144384080 | 144390028 | 144401198 | 144401307 | 3.78E−05 | 4.44E−03 | −5.200E−02 |
| 108040 | COA1 | chr7 | − | 43657215 | 43657269 | 43650611 | 43650712 | 43729428 | 43729505 | 5.18E−04 | 3.41E−02 | −5.200E−02 |
| 129266 | SLC44A2 | chr19 | + | 10635430 | 10635515 | 10634755 | 10635073 | 10636322 | 10636585 | 2.76E−04 | 2.07E−02 | −5.200E−02 |
| 137512 | BCLAF3 | chrX | − | 19935808 | 19935898 | 19929784 | 19929940 | 19937417 | 19937532 | 1.12E−04 | 1.04E−02 | −5.200E−02 |
| 155382 | TRMT2B | chrX | − | 101041316 | 101041371 | 101037916 | 101038051 | 101051250 | 101051366 | 1.46E−06 | 3.13E−04 | −5.200E−02 |
| 161968 | PITPNM2 | chr12 | − | 123013827 | 123014042 | 123012612 | 123012734 | 123034512 | 123034654 | 6.47E−04 | 4.07E−02 | −5.200E−02 |
| 8169 | IFT52 | chr20 | + | 43605001 | 43605216 | 43604182 | 43604258 | 43613849 | 43613976 | 7.27E−04 | 4.46E−02 | −5.100E−02 |
| 34316 | P4HA1 | chr10 | − | 73044980 | 73045051 | 73043886 | 73043957 | 73046924 | 73047101 | 1.24E−04 | 1.12E−02 | −5.100E−02 |
| 42303 | NUTM2A-AS1 | chr10 | − | 87241181 | 87245855 | 87207623 | 87207808 | 87246114 | 87246212 | 1.07E−06 | 2.46E−04 | −5.100E−02 |
| 142155 | SIGIRR | chr11 | − | 406875 | 406993 | 406348 | 406538 | 407061 | 407164 | 9.72E−05 | 9.25E−03 | −5.100E−02 |
| 160846 | ACAD10 | chr12 | + | 111702161 | 111702310 | 111692696 | 111692896 | 111705737 | 111705932 | 1.85E−04 | 1.53E−02 | −5.100E−02 |
| 162990 | ZMYM5 | chr13 | − | 19837655 | 19837821 | 19835476 | 19835689 | 19851688 | 19852190 | 1.92E−08 | 9.13E−06 | −5.100E−02 |
| 195502 | AC243960.1 | chr19 | + | 41551179 | 41551494 | 41549519 | 41549552 | 41553705 | 41553746 | 4.10E−04 | 2.85E−02 | −5.100E−02 |
| 1334 | ZMYM3 | chrX | − | 71251177 | 71251250 | 71250431 | 71250726 | 71251557 | 71251601 | 1.94E−07 | 6.19E−05 | −5.000E−02 |
| 22750 | VNN2 | chr6 | − | 132752672 | 132752749 | 132751144 | 132751518 | 132755842 | 132756035 | 1.45E−04 | 1.27E−02 | −5.000E−02 |
| 32871 | SERINC5 | chr5 | − | 80169334 | 80169546 | 80166382 | 80166478 | 80174953 | 80175047 | 3.73E−04 | 2.65E−02 | −5.000E−02 |
| 70248 | POGLUT3 | chr11 | − | 108479300 | 108479495 | 108477606 | 108477711 | 108486156 | 108486440 | 1.75E−06 | 3.65E−04 | −5.000E−02 |
| 135451 | LAIR1 | chr19 | − | 54364294 | 54364486 | 54360915 | 54361209 | 54370261 | 54370558 | 7.39E−04 | 4.51E−02 | −5.000E−02 |
| 182832 | BAZ2A | chr12 | − | 56615013 | 56615128 | 56613952 | 56614138 | 56615218 | 56615607 | 4.84E−04 | 3.23E−02 | −5.000E−02 |
| 196399 | FCGRT | chr19 | + | 49525456 | 49525561 | 49514210 | 49514486 | 49526009 | 49526428 | 3.56E−04 | 2.55E−02 | −5.000E−02 |
| 3755 | GGA1 | chr22 | + | 37613118 | 37613191 | 37608844 | 37608903 | 37614189 | 37614195 | 8.63E−07 | 2.09E−04 | 5.000E−02 |
| 62394 | TMEM161B-AS1 | chr5 | + | 88285743 | 88285851 | 88283009 | 88283086 | 88287438 | 88287616 | 3.83E−04 | 2.71E−02 | 5.000E−02 |
| 68317 | KRIT1 | chr7 | − | 92245426 | 92245595 | 92242033 | 92242137 | 92245789 | 92245920 | 2.69E−06 | 5.17E−04 | 5.000E−02 |
| 83520 | SAMD4B | chr19 | + | 39351816 | 39352374 | 39342467 | 39342576 | 39354005 | 39354066 | 2.97E−05 | 3.65E−03 | 5.000E−02 |
| 113132 | FRG1CP | chr20 | − | 28590556 | 28590614 | 28588642 | 28588757 | 28600391 | 28600461 | 2.46E−06 | 4.80E−04 | 5.000E−02 |
| 124021 | ACADVL | chr17 | + | 7220463 | 7220529 | 7219843 | 7220197 | 7220603 | 7220676 | 3.13E−04 | 2.31E−02 | 5.000E−02 |
| 128753 | KIF27 | chr9 | − | 83880296 | 83880494 | 83870518 | 83870632 | 83887040 | 83887196 | 2.29E−05 | 2.97E−03 | 5.000E−02 |
| 135502 | NBPF12 | chr1 | + | 146989573 | 146989746 | 146988840 | 146988949 | 146991112 | 146991285 | 1.43E−10 | 1.41E−07 | 5.000E−02 |
| 136634 | NUP62 | chr19 | − | 49927693 | 49927848 | 49908104 | 49909884 | 49929349 | 49929763 | 1.47E−04 | 1.28E−02 | 5.000E−02 |
| 150732 | CEP295 | chr11 | + | 93724253 | 93724375 | 93722501 | 93723289 | 93725650 | 93725831 | 1.70E−12 | 3.16E−09 | 5.000E−02 |
| 153486 | RHNO1 | chr12 | + | 2885282 | 2885534 | 2877222 | 2877282 | 2887910 | 2887934 | 1.91E−04 | 1.57E−02 | 5.000E−02 |
| 175578 | FANCI | chr15 | + | 89293832 | 89293997 | 89292941 | 89293063 | 89299799 | 89299966 | 2.17E−04 | 1.72E−02 | 5.000E−02 |
| 178089 | RMDN1 | chr8 | − | 86474819 | 86474914 | 86472352 | 86472479 | 86477293 | 86477324 | 5.68E−10 | 4.36E−07 | 5.000E−02 |
| 182903 | UBA52 | chr19 | + | 18573292 | 18573403 | 18571801 | 18571887 | 18574869 | 18574972 | 6.90E−04 | 4.28E−02 | 5.000E−02 |
| 188722 | ELF4 | chrX | − | 130081255 | 130081312 | 130074580 | 130074752 | 130110619 | 130110716 | 5.63E−04 | 3.64E−02 | 5.000E−02 |
| 49460 | MVK | chr12 | + | 109579801 | 109579946 | 109575997 | 109576145 | 109581394 | 109581535 | 9.67E−05 | 9.23E−03 | 5.100E−02 |
| 51570 | WASHC2A | chr10 | + | 50127159 | 50127222 | 50126056 | 50126179 | 50129418 | 50129784 | 4.22E−05 | 4.84E−03 | 5.100E−02 |
| 52127 | TMEM79 | chr1 | + | 156285183 | 156285267 | 156282934 | 156283065 | 156286259 | 156286473 | 4.51E−05 | 5.11E−03 | 5.100E−02 |
| 62120 | BANP | chr16 | + | 88027482 | 88027650 | 88018427 | 88018667 | 88076589 | 88076661 | 1.03E−10 | 1.05E−07 | 5.100E−02 |
| 63583 | EBLN3P | chr9 | + | 37080341 | 37080536 | 37079873 | 37080034 | 37086667 | 37087995 | 5.97E−06 | 9.81E−04 | 5.100E−02 |
| 84487 | ITGB3BP | chr1 | − | 63510064 | 63510181 | 63508527 | 63508570 | 63523128 | 63523194 | 6.66E−16 | 2.79E−12 | 5.100E−02 |
| 94155 | TMEM267 | chr5 | − | 43453657 | 43454043 | 43444251 | 43446557 | 43483821 | 43483885 | 7.62E−04 | 4.63E−02 | 5.100E−02 |
| 113668 | SLC25A37 | chr8 | + | 23543087 | 23543211 | 23541469 | 23541856 | 23566107 | 23566378 | 3.38E−04 | 2.45E−02 | 5.100E−02 |
| 146758 | SERPING1 | chr11 | + | 57599863 | 57600377 | 57598248 | 57598321 | 57602034 | 57602169 | 4.69E−04 | 3.15E−02 | 5.100E−02 |
| 163152 | AC087632.2 | chr15 | − | 64207508 | 64207594 | 64204864 | 64204949 | 64213889 | 64214124 | 7.37E−04 | 4.51E−02 | 5.100E−02 |
| 168889 | TGFB3 | chr14 | − | 75960268 | 75960386 | 75959288 | 75959345 | 75960922 | 75960957 | 3.21E−05 | 3.89E−03 | 5.100E−02 |
| 20399 | TPT1-AS1 | chr13 | + | 45383759 | 45383838 | 45378529 | 45380001 | 45389734 | 45389747 | 9.55E−06 | 1.44E−03 | 5.200E−02 |
| 75239 | LGMN | chr14 | − | 92706482 | 92706653 | 92703808 | 92704361 | 92709671 | 92709872 | 4.01E−06 | 7.07E−04 | 5.200E−02 |
| 75816 | INO80C | chr18 | − | 35479299 | 35479411 | 35478281 | 35478349 | 35497718 | 35497933 | 1.46E−09 | 9.93E−07 | 5.200E−02 |
| 75817 | INO80C | chr18 | − | 35480452 | 35480563 | 35478281 | 35478349 | 35497718 | 35497933 | 1.92E−05 | 2.59E−03 | 5.200E−02 |
| 93518 | TAF11 | chr6 | − | 34879966 | 34880063 | 34877461 | 34878720 | 34882931 | 34883080 | 4.57E−04 | 3.10E−02 | 5.200E−02 |
| 95344 | ATRIP | chr3 | + | 48461360 | 48461445 | 48460708 | 48460799 | 48463744 | 48463881 | 7.60E−04 | 4.62E−02 | 5.200E−02 |
| 98851 | NCOR2 | chr12 | − | 124457105 | 124457162 | 124449814 | 124449867 | 124466172 | 124466286 | 5.36E−05 | 5.79E−03 | 5.200E−02 |
| 102450 | GTDC1 | chr2 | − | 143952772 | 143952843 | 143951981 | 143952073 | 144007181 | 144007535 | 7.33E−04 | 4.49E−02 | 5.200E−02 |
| 116592 | CPVL | chr7 | − | 29066022 | 29066121 | 29030576 | 29030759 | 29086483 | 29086550 | 1.65E−06 | 3.47E−04 | 5.200E−02 |
| 123651 | PVT1 | chr8 | + | 128070159 | 128070272 | 128010317 | 128010444 | 128082752 | 128082848 | 2.50E−07 | 7.46E−05 | 5.200E−02 |
| 176961 | RNPS1 | chr16 | − | 2267174 | 2267218 | 2264572 | 2264760 | 2267662 | 2267848 | 3.71E−06 | 6.64E−04 | 5.200E−02 |
| 190876 | A1BG-AS1 | chr19 | + | 58353320 | 58353474 | 58347750 | 58347844 | 58353713 | 58353857 | 1.29E−06 | 2.85E−04 | 5.200E−02 |
| 193099 | PCBP1-AS1 | chr2 | − | 70053730 | 70053792 | 70051044 | 70051305 | 70055655 | 70055749 | 1.30E−04 | 1.16E−02 | 5.200E−02 |
| 30258 | CAMLG | chr5 | + | 134741062 | 134741523 | 134738547 | 134738792 | 134743986 | 134744052 | 1.57E−06 | 3.31E−04 | 5.300E−02 |
| 59187 | TMBIM1 | chr2 | − | 218285762 | 218285938 | 218281939 | 218282181 | 218292465 | 218292529 | 5.83E−04 | 3.74E−02 | 5.300E−02 |
| 117321 | SLC15A2 | chr3 | + | 121940383 | 121940488 | 121939190 | 121939495 | 121940830 | 121940982 | 3.62E−07 | 1.01E−04 | 5.300E−02 |
| 125879 | DENND4C | chr9 | + | 19371755 | 19371820 | 19361845 | 19361963 | 19372036 | 19372594 | 8.89E−07 | 2.15E−04 | 5.300E−02 |
| 143112 | MAP3K20 | chr2 | + | 173090997 | 173091190 | 173075841 | 173076002 | 173169804 | 173169892 | 2.15E−14 | 6.53E−11 | 5.300E−02 |
| 177313 | RAB4A | chr1 | + | 229288728 | 229288843 | 229271110 | 229271370 | 229295847 | 229295910 | 4.13E−04 | 2.87E−02 | 5.300E−02 |
| 179713 | NOD2 | chr16 | + | 50707854 | 50707960 | 50693587 | 50693662 | 50710557 | 50710791 | 8.66E−15 | 3.11E−11 | 5.300E−02 |
| 19787 | ERMARD | chr6 | + | 169753943 | 169754032 | 169751621 | 169751663 | 169755282 | 169755422 | 3.14E−10 | 2.68E−07 | 5.400E−02 |
| 27422 | ZNF354B | chr5 | + | 178885613 | 178885737 | 178883864 | 178884518 | 178887608 | 178888122 | 4.82E−11 | 5.72E−08 | 5.400E−02 |
| 74276 | NOTCH2 | chr1 | − | 119948413 | 119948566 | 119941525 | 119941754 | 119950723 | 119950837 | 2.89E−12 | 4.93E−09 | 5.400E−02 |
| 80946 | IP6K2 | chr3 | − | 48715314 | 48715527 | 48693520 | 48695421 | 48717156 | 48717188 | 6.51E−04 | 4.09E−02 | 5.400E−02 |
| 91414 | HLTF | chr3 | − | 149041489 | 149041668 | 149040030 | 149040156 | 149042165 | 149042290 | 4.24E−05 | 4.86E−03 | 5.400E−02 |
| 113349 | TRAF3IP2-AS1 | chr6 | + | 111574620 | 111574802 | 111565250 | 111565282 | 111576361 | 111576470 | 3.86E−04 | 2.73E−02 | 5.400E−02 |
| 119504 | CCNL1 | chr3 | − | 157157010 | 157157083 | 157153035 | 157153156 | 157158865 | 157158975 | 5.73E−04 | 3.69E−02 | 5.400E−02 |
| 150405 | SYTL2 | chr11 | − | 85714412 | 85714507 | 85709330 | 85709500 | 85718789 | 85718843 | 4.65E−05 | 5.22E−03 | 5.400E−02 |
| 154152 | DGUOK | chr2 | + | 73938909 | 73939022 | 73926825 | 73927052 | 73957124 | 73957240 | 5.95E−04 | 3.81E−02 | 5.400E−02 |
| 13612 | AGPAT5 | chr8 | + | 6747669 | 6747828 | 6732560 | 6732650 | 6755050 | 6755174 | 1.12E−04 | 1.04E−02 | 5.500E−02 |
| 22803 | VNN2 | chr6 | − | 132760631 | 132760803 | 132757670 | 132757759 | 132763382 | 132763431 | 2.11E−04 | 1.68E−02 | 5.500E−02 |
| 27427 | R3HCC1L | chr10 | + | 98162882 | 98162975 | 98134670 | 98134706 | 98163292 | 98163397 | 9.11E−07 | 2.19E−04 | 5.500E−02 |
| 128743 | KIF27 | chr9 | − | 83859155 | 83859371 | 83853628 | 83853835 | 83867683 | 83867860 | 3.06E−06 | 5.73E−04 | 5.500E−02 |
| 132199 | LINC00963 | chr9 | + | 129493415 | 129493595 | 129488544 | 129489299 | 129513418 | 129513686 | 5.11E−05 | 5.60E−03 | 5.500E−02 |
| 168341 | JAK3 | chr19 | − | 17839179 | 17839293 | 17838269 | 17838390 | 17839476 | 17839663 | 4.67E−04 | 3.15E−02 | 5.500E−02 |
| 178114 | PPP4R1L | chr20 | − | 58236153 | 58236274 | 58235239 | 58235374 | 58238210 | 58238311 | 9.49E−07 | 2.26E−04 | 5.500E−02 |
| 183139 | HM13 | chr20 | + | 31567639 | 31567740 | 31566209 | 31566295 | 31568077 | 31568280 | 3.04E−05 | 3.72E−03 | 5.500E−02 |
| 183141 | HM13 | chr20 | + | 31567639 | 31567806 | 31566153 | 31566295 | 31568077 | 31568280 | 2.45E−04 | 1.88E−02 | 5.500E−02 |
| 195115 | GMFG | chr19 | − | 39335260 | 39335310 | 39329543 | 39329626 | 39335973 | 39336017 | 7.40E−07 | 1.84E−04 | 5.500E−02 |
| 3749 | GGA1 | chr22 | + | 37613023 | 37613191 | 37608844 | 37608903 | 37614189 | 37614274 | 1.38E−08 | 6.96E−06 | 5.600E−02 |
| 18232 | CCM2 | chr7 | + | 45070093 | 45070168 | 45069825 | 45069961 | 45070388 | 45071872 | 2.01E−04 | 1.62E−02 | 5.600E−02 |
| 30267 | LY96 | chr8 | + | 74004795 | 74004885 | 73991391 | 73991554 | 74010000 | 74010129 | 2.58E−07 | 7.66E−05 | 5.600E−02 |
| 55309 | EIF4G3 | chr1 | − | 21002712 | 21002808 | 20981047 | 20981227 | 21050865 | 21050994 | 4.69E−13 | 1.03E−09 | 5.600E−02 |
| 85132 | SNX25 | chr4 | + | 185258847 | 185259064 | 185247293 | 185247378 | 185264437 | 185264498 | 2.92E−06 | 5.55E−04 | 5.600E−02 |
| 97359 | METTL15 | chr11 | + | 28122141 | 28122180 | 28113317 | 28113604 | 28211061 | 28211198 | 2.46E−06 | 4.80E−04 | 5.600E−02 |
| 122669 | CEP290 | chr12 | − | 88054339 | 88054413 | 88053651 | 88053746 | 88055575 | 88055717 | 2.80E−04 | 2.10E−02 | 5.600E−02 |
| 129128 | MAP4 | chr3 | − | 47867245 | 47867338 | 47857430 | 47857512 | 47870857 | 47871105 | 1.96E−05 | 2.62E−03 | 5.600E−02 |
| 180825 | DPEP2 | chr16 | − | 67992949 | 67993257 | 67991114 | 67991184 | 67999395 | 67999518 | 1.30E−05 | 1.88E−03 | 5.600E−02 |
| 17373 | TCEA3 | chr1 | − | 23408663 | 23408726 | 23397791 | 23397955 | 23417248 | 23417390 | 1.82E−09 | 1.21E−06 | 5.700E−02 |
| 36901 | ST6GALNAC4 | chr9 | − | 127909950 | 127910528 | 127908317 | 127908581 | 127912267 | 127912680 | 1.60E−08 | 7.79E−06 | 5.700E−02 |
| 54600 | CASP5 | chr11 | − | 105003273 | 105003383 | 105002027 | 105002201 | 105023129 | 105023136 | 1.26E−04 | 1.13E−02 | 5.700E−02 |
| 82051 | DLGAP4 | chr20 | + | 36465264 | 36465367 | 36461467 | 36461510 | 36496704 | 36497066 | 2.58E−08 | 1.18E−05 | 5.700E−02 |
| 96608 | RBIS | chr8 | − | 85219235 | 85219360 | 85217385 | 85217502 | 85220305 | 85220353 | 3.77E−12 | 6.29E−09 | 5.700E−02 |
| 103734 | LUCAT1 | chr5 | − | 91303872 | 91303971 | 91301903 | 91303161 | 91305679 | 91305771 | 4.81E−05 | 5.36E−03 | 5.700E−02 |
| 141378 | PSTK | chr10 | + | 122982732 | 122983024 | 122980039 | 122980695 | 122983271 | 122983470 | 1.29E−10 | 1.27E−07 | 5.700E−02 |
| 162316 | GLT1D1 | chr12 | + | 128914932 | 128914989 | 128899235 | 128899287 | 128945325 | 128945369 | 3.25E−05 | 3.92E−03 | 5.700E−02 |
| 166728 | GTF21 | chr7 | + | 74744138 | 74744250 | 74743448 | 74743520 | 74744757 | 74744941 | 7.66E−05 | 7.71E−03 | 5.700E−02 |
| 169278 | NRDE2 | chr14 | − | 90317703 | 90317820 | 90316586 | 90316811 | 90318004 | 90318113 | 3.30E−06 | 6.05E−04 | 5.700E−02 |
| 171253 | ST3GAL2 | chr16 | − | 70400695 | 70401094 | 70399449 | 70399533 | 70422943 | 70423073 | 1.71E−05 | 2.36E−03 | 5.700E−02 |
| 172656 | SNAP23 | chr15 | + | 42529674 | 42529819 | 42515236 | 42515354 | 42545208 | 42545356 | 7.93E−05 | 7.92E−03 | 5.700E−02 |
| 178518 | AC138894.1 | chr16 | − | 28491481 | 28491513 | 28489289 | 28489386 | 28491713 | 28491835 | 3.32E−04 | 2.42E−02 | 5.700E−02 |
| 180943 | LMAN2L | chr2 | − | 96733518 | 96733601 | 96711863 | 96712025 | 96734408 | 96734526 | 1.71E−05 | 2.36E−03 | 5.700E−02 |
| 192035 | CERS4 | chr19 | + | 8257878 | 8257985 | 8256948 | 8257077 | 8261929 | 8262418 | 1.14E−04 | 1.05E−02 | 5.700E−02 |
| 19558 | PGAP2 | chr11 | + | 3822908 | 3822993 | 3811249 | 3811424 | 3823882 | 3824135 | 2.44E−04 | 1.87E−02 | 5.800E−02 |
| 107624 | SLC12A2 | chr5 | + | 128177104 | 128177152 | 128174540 | 128174666 | 128178566 | 128178689 | 1.89E−05 | 2.56E−03 | 5.800E−02 |
| 31791 | CAMK4 | chr5 | + | 111376859 | 111376942 | 111344023 | 111344102 | 111394709 | 111394749 | 1.77E−05 | 2.43E−03 | 5.900E−02 |
| 40982 | ABRAXAS1 | chr4 | − | 83470202 | 83470396 | 83469031 | 83469151 | 83476642 | 83476679 | 3.64E−05 | 4.29E−03 | 5.900E−02 |
| 77387 | FCRL2 | chr1 | − | 157770408 | 157770666 | 157766854 | 157766971 | 157775774 | 157775795 | 3.04E−10 | 2.63E−07 | 5.900E−02 |
| 104000 | TANGO2 | chr22 | + | 20061529 | 20061683 | 20055942 | 20056013 | 20063337 | 20063411 | 4.14E−04 | 2.87E−02 | 5.900E−02 |
| 112543 | GGCT | chr7 | − | 30500535 | 30500681 | 30498802 | 30498938 | 30504568 | 30504799 | 4.15E−04 | 2.88E−02 | 5.900E−02 |
| 151459 | AP001781.2 | chr11 | − | 111876480 | 111876639 | 111875697 | 111876104 | 111876807 | 111876844 | 7.12E−08 | 2.70E−05 | 5.900E−02 |
| 179730 | NOD2 | chr16 | + | 50716890 | 50716974 | 50710557 | 50712373 | 50719924 | 50720008 | 9.32E−05 | 8.97E−03 | 5.900E−02 |
| 186409 | FYN | chr6 | − | 111754433 | 111754677 | 111719855 | 111720062 | 111759844 | 111759925 | 2.00E−05 | 2.66E−03 | 5.900E−02 |
| 189083 | CYBC1 | chr17 | − | 82449430 | 82449956 | 82449173 | 82449292 | 82450699 | 82450737 | 8.33E−04 | 4.97E−02 | 5.900E−02 |
| 190331 | CCDC191 | chr3 | − | 114046590 | 114046732 | 114042702 | 114042846 | 114056376 | 114056549 | 3.33E−04 | 2.42E−02 | 5.900E−02 |
| 9140 | ABHD12 | chr20 | − | 25314924 | 25314970 | 25309445 | 25309575 | 25323324 | 25323430 | 5.56E−13 | 1.15E−09 | 6.000E−02 |
| 22272 | FOPNL | chr16 | − | 15873490 | 15873627 | 15865718 | 15867516 | 15884007 | 15884205 | 8.10E−04 | 4.86E−02 | 6.000E−02 |
| 86241 | NEXN | chr1 | + | 77935822 | 77936044 | 77933047 | 77933479 | 77942022 | 77942208 | 6.34E−09 | 3.58E−06 | 6.000E−02 |
| 143186 | HFE | chr6 | + | 26090840 | 26091104 | 26087280 | 26087516 | 26091313 | 26091589 | 3.03E−09 | 1.85E−06 | 6.000E−02 |
| 155385 | TRMT2B | chrX | − | 101041316 | 101041371 | 101037916 | 101038051 | 101051816 | 101052054 | 2.02E−08 | 9.45E−06 | 6.000E−02 |
| 168705 | ABCD4 | chr14 | − | 74299547 | 74299675 | 74297835 | 74298069 | 74302874 | 74302910 | 1.30E−04 | 1.16E−02 | 6.000E−02 |
| 195508 | AC243960.1 | chr19 | + | 41553705 | 41553746 | 41549519 | 41549552 | 41554950 | 41555099 | 1.76E−07 | 5.74E−05 | 6.000E−02 |
| 36506 | PSMG4 | chr6 | + | 3264173 | 3264325 | 3263683 | 3263759 | 3267590 | 3267920 | 1.27E−13 | 3.44E−10 | 6.100E−02 |
| 43880 | IKBKG | chrX | + | 154560406 | 154560559 | 154558531 | 154558650 | 154561687 | 154561763 | 4.93E−08 | 2.01E−05 | 6.100E−02 |
| 66157 | TRMT61B | chr2 | − | 28852407 | 28852499 | 28850327 | 28850405 | 28861117 | 28861308 | 6.76E−09 | 3.78E−06 | 6.100E−02 |
| 71196 | IGHG3 | chr14 | − | 105770292 | 105770337 | 105770104 | 105770149 | 105770480 | 105770525 | 5.40E−11 | 6.16E−08 | 6.100E−02 |
| 113217 | ING3 | chr7 | + | 120964741 | 120964838 | 120955558 | 120955624 | 120966625 | 120966697 | 6.96E−04 | 4.30E−02 | 6.100E−02 |
| 118116 | RPL32P3 | chr3 | − | 129396216 | 129396420 | 129393634 | 129393795 | 129397039 | 129397140 | 2.70E−04 | 2.04E−02 | 6.100E−02 |
| 126580 | ABHD14A-ACY1 | chr3 | + | 51988523 | 51988603 | 51984046 | 51984158 | 51988765 | 51988789 | 7.03E−07 | 1.76E−04 | 6.100E−02 |
| 148701 | TBCD | chr17 | + | 82909284 | 82909307 | 82903404 | 82903478 | 82911757 | 82911765 | 1.74E−04 | 1.46E−02 | 6.100E−02 |
| 172093 | RPAIN | chr17 | + | 5426235 | 5426299 | 5422768 | 5422829 | 5432541 | 5432876 | 6.42E−04 | 4.05E−02 | 6.100E−02 |
| 178559 | SULT1A1 | chr16 | − | 28609930 | 28610191 | 28606950 | 28607077 | 28620062 | 28620133 | 1.89E−09 | 1.24E−06 | 6.100E−02 |
| 6977 | TCFL5 | chr20 | − | 62857394 | 62857638 | 62854015 | 62854157 | 62860124 | 62860308 | 1.07E−05 | 1.59E−03 | 6.200E−02 |
| 11532 | TCF25 | chr16 | + | 89893788 | 89893858 | 89892192 | 89892275 | 89895082 | 89895137 | 1.11E−16 | 5.28E−13 | 6.200E−02 |
| 75605 | COX20 | chr1 | + | 244841943 | 244842058 | 244835657 | 244835756 | 244842194 | 244842258 | 4.29E−04 | 2.95E−02 | 6.200E−02 |
| 144397 | SERGEF | chr11 | − | 18010074 | 18010139 | 18007940 | 18008076 | 18012950 | 18013012 | 1.95E−12 | 3.53E−09 | 6.200E−02 |
| 178652 | KIAA2026 | chr9 | − | 5910614 | 5910707 | 5881595 | 5881675 | 5913857 | 5913860 | 1.48E−07 | 5.01E−05 | 6.200E−02 |
| 7842 | CD40 | chr20 | + | 46121723 | 46121898 | 46118372 | 46118394 | 46122232 | 46122358 | 2.44E−10 | 2.19E−07 | 6.300E−02 |
| 12001 | KIR2DL1 | chr19 | + | 54780088 | 54780166 | 54778611 | 54778662 | 54782921 | 54783023 | 7.23E−06 | 1.15E−03 | 6.300E−02 |
| 25070 | GPR141 | chr7 | + | 37709717 | 37709799 | 37685459 | 37685583 | 37713341 | 37713510 | 1.27E−04 | 1.14E−02 | 6.300E−02 |
| 102608 | LRRC37B | chr17 | + | 32027768 | 32027840 | 32024710 | 32024782 | 32034909 | 32034981 | 2.39E−07 | 7.24E−05 | 6.300E−02 |
| 128569 | TMEM44-AS1 | chr3 | + | 194589020 | 194589142 | 194584010 | 194584448 | 194589473 | 194589626 | 8.89E−05 | 8.68E−03 | 6.300E−02 |
| 137473 | TYSND1 | chr10 | − | 70142667 | 70142853 | 70137980 | 70140141 | 70145420 | 70146700 | 1.98E−08 | 9.32E−06 | 6.300E−02 |
| 138994 | FGR | chr1 | − | 27621558 | 27621657 | 27616856 | 27617006 | 27635064 | 27635185 | 2.75E−04 | 2.06E−02 | 6.300E−02 |
| 181805 | ZNF133 | chr20 | + | 18305680 | 18305807 | 18288282 | 18288604 | 18306297 | 18306393 | 3.37E−08 | 1.47E−05 | 6.300E−02 |
| 5424 | BCL2L13 | chr22 | + | 17696140 | 17696210 | 17683213 | 17683321 | 17702242 | 17702386 | 3.41E−09 | 2.05E−06 | 6.400E−02 |
| 80480 | PMS2 | chr7 | − | 6005891 | 6006031 | 6003971 | 6004058 | 6008996 | 6009019 | 8.86E−06 | 1.36E−03 | 6.400E−02 |
| 139668 | ARHGAP19 | chr10 | − | 97256317 | 97256404 | 97246271 | 97246337 | 97259401 | 97259628 | 4.64E−06 | 7.96E−04 | 6.400E−02 |
| 143407 | TRIM34 | chr11 | + | 5642816 | 5642853 | 5642405 | 5642506 | 5643143 | 5643809 | 2.54E−07 | 7.57E−05 | 6.400E−02 |
| 146263 | ZBTB8OS | chr1 | − | 32634767 | 32634792 | 32633950 | 32634072 | 32650428 | 32650550 | 9.81E−07 | 2.31E−04 | 6.400E−02 |
| 148979 | RESF1 | chr12 | + | 31980877 | 31985957 | 31970188 | 31970356 | 31992377 | 31993107 | 4.45E−09 | 2.59E−06 | 6.400E−02 |
| 153877 | CHD4 | chr12 | − | 6580581 | 6580679 | 6578845 | 6578917 | 6581037 | 6581173 | 1.97E−05 | 2.63E−03 | 6.400E−02 |
| 181799 | ZNF133 | chr20 | + | 18305667 | 18305807 | 18288282 | 18288604 | 18306297 | 18306393 | 4.93E−08 | 2.01E−05 | 6.400E−02 |
| 192056 | RBM23 | chr14 | − | 22905605 | 22905659 | 22905335 | 22905453 | 22906194 | 22906368 | 1.87E−10 | 1.74E−07 | 6.400E−02 |
| 193194 | PCBP1-AS1 | chr2 | − | 70059642 | 70059680 | 70055616 | 70055910 | 70085546 | 70085574 | 4.86E−04 | 3.24E−02 | 6.400E−02 |
| 35288 | TMEM116 | chr12 | − | 111978735 | 111978787 | 111943264 | 111943369 | 111991757 | 111991889 | 1.38E−04 | 1.21E−02 | 6.500E−02 |
| 76752 | PI4KB | chr1 | − | 151324744 | 151324917 | 151307573 | 151307801 | 151326171 | 151326373 | 9.31E−07 | 2.23E−04 | 6.500E−02 |
| 176282 | SNRPA1 | chr15 | − | 101291961 | 101292040 | 101287655 | 101287702 | 101295096 | 101295204 | 1.55E−04 | 1.33E−02 | 6.500E−02 |
| 193110 | PCBP1-AS1 | chr2 | − | 70053730 | 70053812 | 70051202 | 70051305 | 70055655 | 70055910 | 6.79E−04 | 4.23E−02 | 6.500E−02 |
| 59613 | HMOX2 | chr16 | + | 4483636 | 4483754 | 4476339 | 4476487 | 4505483 | 4505610 | 1.07E−07 | 3.79E−05 | 6.600E−02 |
| 90406 | GMDS-DT | chr6 | + | 2269697 | 2269799 | 2263600 | 2263684 | 2398576 | 2398778 | 2.75E−08 | 1.26E−05 | 6.600E−02 |
| 98608 | IL18R1 | chr2 | + | 102367824 | 102368068 | 102362632 | 102362718 | 102371952 | 102372118 | 5.23E−04 | 3.43E−02 | 6.600E−02 |
| 4741 | UPB1 | chr22 | + | 24523618 | 24523773 | 24520386 | 24520468 | 24525710 | 24526668 | 2.78E−06 | 5.31E−04 | 6.700E−02 |
| 17041 | ZNF138 | chr7 | + | 64815575 | 64815653 | 64814917 | 64815044 | 64830939 | 64831076 | 6.47E−07 | 1.64E−04 | 6.700E−02 |
| 23911 | GATC | chr12 | + | 120454932 | 120455030 | 120446656 | 120446829 | 120457075 | 120457179 | 1.41E−12 | 2.64E−09 | 6.700E−02 |
| 26446 | PIK3C2B | chr1 | − | 204433792 | 204433949 | 204433315 | 204433425 | 204434438 | 204434608 | 9.40E−05 | 9.02E−03 | 6.700E−02 |
| 38089 | UPF3A | chr13 | + | 114286301 | 114286400 | 114282020 | 114282127 | 114286518 | 114286629 | 5.83E−07 | 1.51E−04 | 6.700E−02 |
| 64980 | ANGEL2 | chr1 | − | 213013092 | 213013418 | 213008209 | 213008466 | 213015225 | 213015493 | 1.14E−06 | 2.60E−04 | 6.700E−02 |
| 68316 | KRIT1 | chr7 | − | 92245426 | 92245574 | 92242033 | 92242137 | 92245789 | 92245923 | 5.06E−05 | 5.56E−03 | 6.700E−02 |
| 153879 | CHD4 | chr12 | − | 6580581 | 6580699 | 6578845 | 6578917 | 6581037 | 6581173 | 4.43E−05 | 5.02E−03 | 6.700E−02 |
| 15337 | SRPK2 | chr7 | − | 105117366 | 105117511 | 105115268 | 105115503 | 105117707 | 105118022 | 7.42E−07 | 1.84E−04 | 6.800E−02 |
| 104391 | EVA1C | chr21 | + | 32457596 | 32457720 | 32452184 | 32453508 | 32467695 | 32467848 | 2.06E−06 | 4.15E−04 | 6.800E−02 |
| 145429 | ACCS | chr11 | + | 44082064 | 44082192 | 44081178 | 44081320 | 44083168 | 44083208 | 3.48E−06 | 6.33E−04 | 6.800E−02 |
| 26488 | DDX60L | chr4 | − | 168377727 | 168377843 | 168375376 | 168375524 | 168378353 | 168378475 | 1.45E−04 | 1.26E−02 | 6.900E−02 |
| 96609 | RBIS | chr8 | − | 85219235 | 85219363 | 85217385 | 85217502 | 85220305 | 85220384 | 3.62E−08 | 1.55E−05 | 6.900E−02 |
| 153405 | N4BP2L2 | chr13 | − | 32537005 | 32537027 | 32527407 | 32527532 | 32538777 | 32538813 | 3.94E−04 | 2.77E−02 | 6.900E−02 |
| 193117 | PCBP1-AS1 | chr2 | − | 70053730 | 70053815 | 70051202 | 70051305 | 70055655 | 70055910 | 5.96E−04 | 3.81E−02 | 7.000E−02 |
| 119114 | ERICH6-AS1 | chr3 | + | 150706926 | 150707171 | 150703563 | 150703667 | 150719868 | 150720156 | 9.04E−05 | 8.79E−03 | 7.100E−02 |
| 157398 | ATP5MC2 | chr12 | − | 53669870 | 53670114 | 53665238 | 53665428 | 53672575 | 53672645 | 3.11E−05 | 3.79E−03 | 7.100E−02 |
| 166862 | SRP54-AS1 | chr14 | − | 34963928 | 34964040 | 34954858 | 34954954 | 34969169 | 34969307 | 6.97E−09 | 3.85E−06 | 7.100E−02 |
| 168030 | ZBTB1 | chr14 | + | 64516599 | 64516751 | 64504725 | 64504946 | 64521486 | 64521606 | 7.89E−10 | 5.73E−07 | 7.100E−02 |
| 28087 | DTNBP1 | chr6 | − | 15651312 | 15651408 | 15637743 | 15637804 | 15652086 | 15652140 | 3.31E−05 | 3.99E−03 | 7.200E−02 |
| 82397 | FAM228B | chr2 | + | 24139369 | 24139450 | 24095140 | 24095229 | 24146747 | 24146835 | 2.30E−04 | 1.79E−02 | 7.200E−02 |
| 104251 | METTL6 | chr3 | − | 15415497 | 15415583 | 15411329 | 15411437 | 15415771 | 15415860 | 2.01E−06 | 4.09E−04 | 7.200E−02 |
| 104671 | KPTN | chr19 | − | 47483294 | 47483379 | 47483125 | 47483215 | 47483934 | 47484013 | 1.14E−12 | 2.25E−09 | 7.200E−02 |
| 120068 | POLK | chr5 | + | 75590343 | 75590440 | 75587025 | 75587058 | 75596221 | 75597178 | 1.54E−08 | 7.56E−06 | 7.200E−02 |
| 136235 | PGS1 | chr17 | + | 78392513 | 78392665 | 78378661 | 78378808 | 78398251 | 78398351 | 1.39E−05 | 1.97E−03 | 7.200E−02 |
| 8414 | RPRD1B | chr20 | + | 38040434 | 38040564 | 38033745 | 38034098 | 38057531 | 38057644 | 6.21E−05 | 6.51E−03 | 7.300E−02 |
| 25399 | LINC00426 | chr13 | − | 30367600 | 30367717 | 30340269 | 30341468 | 30372284 | 30372374 | 1.53E−04 | 1.32E−02 | 7.300E−02 |
| 91582 | CD160 | chr1 | + | 145730743 | 145731070 | 145728243 | 145728400 | 145735996 | 145736134 | 1.88E−06 | 3.88E−04 | 7.300E−02 |
| 123048 | TAF2 | chr8 | − | 119801793 | 119802025 | 119797661 | 119797846 | 119803877 | 119804019 | 2.12E−04 | 1.68E−02 | 7.300E−02 |
| 134117 | IL15RA | chr10 | − | 5963742 | 5963841 | 5960366 | 5960567 | 5977404 | 5977492 | 1.26E−04 | 1.14E−02 | 7.300E−02 |
| 138630 | ANXA11 | chr10 | − | 80172806 | 80172869 | 80164052 | 80164143 | 80205342 | 80205572 | 5.72E−08 | 2.25E−05 | 7.300E−02 |
| 14498 | RALGAPB | chr20 | + | 38535085 | 38535207 | 38532729 | 38532859 | 38539775 | 38539958 | 1.10E−05 | 1.63E−03 | 7.400E−02 |
| 93898 | FAM13B | chr5 | − | 138019880 | 138019977 | 138018954 | 138019146 | 138021030 | 138021197 | 2.70E−04 | 2.04E−02 | 7.400E−02 |
| 105694 | KIAA1191 | chr5 | − | 176359810 | 176359918 | 176352621 | 176352748 | 176361601 | 176361735 | 2.41E−04 | 1.86E−02 | 7.400E−02 |
| 180749 | DPEP2 | chr16 | − | 67991114 | 67991184 | 67990820 | 67990939 | 67992063 | 67992193 | 2.15E−04 | 1.71E−02 | 7.400E−02 |
| 190719 | CD226 | chr18 | − | 69873143 | 69873246 | 69842283 | 69842395 | 69895700 | 69896045 | 2.09E−09 | 1.36E−06 | 7.400E−02 |
| 9979 | PTPRA | chr20 | + | 2975214 | 2975241 | 2947981 | 2948024 | 2986764 | 2986849 | 2.49E−04 | 1.90E−02 | 7.500E−02 |
| 48300 | ZNF75D | chrX | − | 135291471 | 135291563 | 135291008 | 135291135 | 135295767 | 135296039 | 3.76E−04 | 2.66E−02 | 7.500E−02 |
| 64546 | NABP1 | chr2 | + | 191682491 | 191682595 | 191681945 | 191682017 | 191683728 | 191683804 | 2.46E−05 | 3.14E−03 | 7.500E−02 |
| 95325 | EME2 | chr16 | + | 1774259 | 1774352 | 1773704 | 1773841 | 1775040 | 1775132 | 1.02E−07 | 3.66E−05 | 7.500E−02 |
| 196286 | SNRNP70 | chr19 | + | 49102113 | 49102185 | 49101389 | 49101471 | 49104633 | 49104735 | 4.81E−04 | 3.21E−02 | 7.500E−02 |
| 50816 | TRPV2 | chr17 | + | 16431783 | 16431850 | 16428816 | 16428982 | 16433573 | 16433698 | 1.30E−04 | 1.16E−02 | 7.600E−02 |
| 155071 | PLBD1-AS1 | chr12 | + | 14614521 | 14614570 | 14612878 | 14612926 | 14619151 | 14619298 | 2.53E−04 | 1.93E−02 | 7.600E−02 |
| 160469 | UBE3B | chr12 | + | 109529889 | 109530072 | 109526357 | 109526416 | 109533465 | 109533558 | 4.07E−04 | 2.84E−02 | 7.600E−02 |
| 169158 | CEP68 | chr2 | + | 65069398 | 65069801 | 65056415 | 65056528 | 65071453 | 65072569 | 4.26E−05 | 4.87E−03 | 7.600E−02 |
| 40984 | ABRAXAS1 | chr4 | − | 83472221 | 83472288 | 83469031 | 83469151 | 83476642 | 83476679 | 4.24E−04 | 2.92E−02 | 7.700E−02 |
| 89687 | NLRP6 | chr11 | + | 280083 | 281839 | 279833 | 279872 | 282701 | 282797 | 1.98E−04 | 1.61E−02 | 7.700E−02 |
| 122109 | RIDA | chr8 | − | 98108645 | 98108768 | 98106271 | 98106326 | 98117031 | 98117110 | 2.44E−04 | 1.87E−02 | 7.700E−02 |
| 160445 | UBE3B | chr12 | + | 109481636 | 109481742 | 109477401 | 109477764 | 109483530 | 109483712 | 3.33E−04 | 2.42E−02 | 7.700E−02 |
| 195503 | AC243960.1 | chr19 | + | 41551179 | 41551494 | 41549519 | 41549552 | 41554950 | 41555099 | 1.83E−07 | 5.90E−05 | 7.700E−02 |
| 17832 | DPY19L1 | chr7 | − | 34957983 | 34958070 | 34955307 | 34955367 | 34966893 | 34966971 | 4.23E−05 | 4.85E−03 | 7.800E−02 |
| 81868 | CLEC4C | chr12 | − | 7741420 | 7741531 | 7737428 | 7737574 | 7746330 | 7746423 | 0.00E+00 | 0.00E+00 | 7.800E−02 |
| 96006 | MADD | chr11 | + | 47273826 | 47273976 | 47270147 | 47270246 | 47274562 | 47274726 | 3.73E−08 | 1.59E−05 | 7.800E−02 |
| 127986 | CBWD3 | chr9 | + | 68266219 | 68266306 | 68264467 | 68264514 | 68268933 | 68268978 | 6.84E−05 | 7.04E−03 | 7.800E−02 |
| 183473 | ANP32A | chr15 | − | 68784396 | 68784595 | 68780409 | 68780473 | 68820697 | 68820868 | 6.85E−08 | 2.64E−05 | 7.800E−02 |
| 188695 | ENDOV | chr17 | + | 80422205 | 80422245 | 80415314 | 80415821 | 80423519 | 80423587 | 1.11E−06 | 2.55E−04 | 7.800E−02 |
| 196383 | FCGRT | chr19 | + | 49521694 | 49521813 | 49513881 | 49514133 | 49524506 | 49524568 | 3.73E−10 | 3.11E−07 | 7.800E−02 |
| 21960 | RNF8 | chr6 | + | 37374619 | 37374709 | 37371511 | 37371574 | 37376925 | 37377033 | 3.64E−06 | 6.56E−04 | 7.900E−02 |
| 53096 | TFB1M | chr6 | − | 155298476 | 155298585 | 155285157 | 155285277 | 155311187 | 155311339 | 1.30E−08 | 6.59E−06 | 7.900E−02 |
| 58405 | LONP2 | chr16 | + | 48256609 | 48256741 | 48252130 | 48252365 | 48258617 | 48258740 | 9.13E−08 | 3.34E−05 | 7.900E−02 |
| 62376 | TMEM161B-AS1 | chr5 | + | 88283009 | 88283086 | 88282041 | 88282106 | 88285740 | 88285851 | 1.55E−11 | 2.10E−08 | 7.900E−02 |
| 62391 | TMEM161B-AS1 | chr5 | + | 88285743 | 88285851 | 88282714 | 88282866 | 88287438 | 88287616 | 4.76E−10 | 3.81E−07 | 7.900E−02 |
| 120054 | POLK | chr5 | + | 75547009 | 75547157 | 75511755 | 75511914 | 75552471 | 75552591 | 1.43E−04 | 1.25E−02 | 7.900E−02 |
| 193123 | PCBP1-AS1 | chr2 | − | 70053730 | 70053994 | 70051202 | 70051305 | 70055655 | 70055910 | 3.43E−04 | 2.47E−02 | 7.900E−02 |
| 193674 | RAB3A | chr19 | − | 18202512 | 18202855 | 18200326 | 18200445 | 18203895 | 18204012 | 1.38E−05 | 1.97E−03 | 7.900E−02 |
| 14656 | SLC37A3 | chr7 | − | 140355667 | 140355764 | 140352061 | 140352146 | 140364407 | 140364491 | 4.06E−06 | 7.13E−04 | 8.000E−02 |
| 36456 | MIR762HG | chr16 | + | 30888713 | 30888958 | 30875778 | 30876118 | 30894547 | 30895136 | 7.46E−04 | 4.55E−02 | 8.000E−02 |
| 72367 | MRS2 | chr6 | + | 24408407 | 24408444 | 24405167 | 24405241 | 24412221 | 24412395 | 1.21E−05 | 1.77E−03 | 8.000E−02 |
| 83568 | TXNDC11 | chr16 | − | 11733981 | 11734079 | 11721576 | 11721670 | 11742476 | 11742547 | 9.01E−11 | 9.60E−08 | 8.000E−02 |
| 103880 | LUCAT1 | chr5 | − | 91313077 | 91313229 | 91264662 | 91264713 | 91313637 | 91313675 | 1.35E−04 | 1.19E−02 | 8.000E−02 |
| 106584 | CERS5 | chr12 | − | 50144768 | 50144969 | 50143951 | 50144057 | 50165821 | 50165943 | 4.45E−08 | 1.83E−05 | 8.000E−02 |
| 62160 | INO80E | chr16 | + | 30001211 | 30003013 | 30000928 | 30001040 | 30005220 | 30005790 | 6.66E−04 | 4.16E−02 | 8.100E−02 |
| 103864 | LUCAT1 | chr5 | − | 91312294 | 91313229 | 91264662 | 91264713 | 91313637 | 91313675 | 1.27E−04 | 1.14E−02 | 8.100E−02 |
| 122426 | AC096887.1 | chr3 | − | 53092368 | 53092618 | 53085749 | 53085852 | 53099380 | 53099453 | 5.55E−16 | 2.38E−12 | 8.100E−02 |
| 158704 | IRAK3 | chr12 | + | 66203710 | 66203893 | 66189213 | 66189432 | 66209455 | 66209520 | 1.16E−04 | 1.07E−02 | 8.100E−02 |
| 185726 | SGCB | chr4 | − | 52033430 | 52033640 | 52029677 | 52029863 | 52038226 | 52038245 | 1.65E−05 | 2.29E−03 | 8.100E−02 |
| 14957 | CEP41 | chr7 | − | 130426645 | 130426759 | 130421852 | 130422007 | 130427954 | 130428018 | 1.52E−04 | 1.31E−02 | 8.200E−02 |
| 19860 | MPC1 | chr6 | − | 166366968 | 166367184 | 166366794 | 166366891 | 166368732 | 166368961 | 8.10E−05 | 8.06E−03 | 8.200E−02 |
| 63495 | PPP2R2D | chr10 | + | 131940030 | 131940196 | 131901237 | 131901330 | 131955683 | 131956555 | 6.03E−10 | 4.54E−07 | 8.200E−02 |
| 88553 | TOGARAM2 | chr2 | + | 29022157 | 29022308 | 29017791 | 29017956 | 29023085 | 29023191 | 2.07E−06 | 4.18E−04 | 8.200E−02 |
| 113670 | SLC25A37 | chr8 | + | 23543087 | 23543211 | 23541686 | 23541839 | 23566107 | 23566378 | 1.09E−07 | 3.84E−05 | 8.200E−02 |
| 139896 | ERLIN1 | chr10 | − | 100164003 | 100164095 | 100156144 | 100156234 | 100174207 | 100174281 | 1.49E−06 | 3.18E−04 | 8.200E−02 |
| 158443 | COQ5 | chr12 | − | 120526456 | 120526546 | 120522213 | 120522363 | 120528939 | 120529140 | 2.19E−05 | 2.87E−03 | 8.200E−02 |
| 179870 | TAF1C | chr16 | − | 84183071 | 84183149 | 84182201 | 84182440 | 84183243 | 84183333 | 1.48E−04 | 1.28E−02 | 8.200E−02 |
| 196287 | SNRNP70 | chr19 | + | 49102113 | 49103587 | 49101389 | 49101471 | 49104633 | 49104735 | 3.99E−04 | 2.80E−02 | 8.200E−02 |
| 38811 | INTS7 | chr1 | − | 211946606 | 211946705 | 211944783 | 211944969 | 211952568 | 211952701 | 5.71E−06 | 9.44E−04 | 8.300E−02 |
| 70760 | DSTYK | chr1 | − | 205160113 | 205160270 | 205159546 | 205159679 | 205161257 | 205161387 | 8.50E−05 | 8.39E−03 | 8.300E−02 |
| 145108 | CD44 | chr11 | + | 35196745 | 35196874 | 35189834 | 35190065 | 35198120 | 35198246 | 4.00E−15 | 1.53E−11 | 8.300E−02 |
| 39328 | RPL5 | chr1 | + | 92833388 | 92833458 | 92832039 | 92832117 | 92836189 | 92836354 | 0.00E+00 | 0.00E+00 | 8.400E−02 |
| 60484 | CCDC138 | chr2 | + | 108856793 | 108856970 | 108846737 | 108846930 | 108873450 | 108873589 | 3.87E−04 | 2.73E−02 | 8.400E−02 |
| 63823 | RMND5B | chr5 | + | 178131236 | 178131376 | 178130995 | 178131054 | 178138107 | 178138258 | 5.18E−04 | 3.41E−02 | 8.400E−02 |
| 78752 | SLC66A2 | chr18 | − | 79947266 | 79947341 | 79943328 | 79943462 | 79949518 | 79949655 | 1.84E−04 | 1.52E−02 | 8.400E−02 |
| 84151 | MAK | chr6 | − | 10830547 | 10830877 | 10818885 | 10818940 | 10838502 | 10838531 | 7.56E−09 | 4.13E−06 | 8.400E−02 |
| 130448 | ABITRAM | chr9 | + | 108939195 | 108939272 | 108936307 | 108936437 | 108950506 | 108950744 | 7.76E−05 | 7.79E−03 | 8.400E−02 |
| 49318 | AC000120.4 | chr7 | − | 92244001 | 92244149 | 92242033 | 92242137 | 92245789 | 92245924 | 1.21E−07 | 4.24E−05 | 8.500E−02 |
| 77273 | TAMM41 | chr3 | − | 11830721 | 11830874 | 11829755 | 11829864 | 11833002 | 11833151 | 3.07E−05 | 3.74E−03 | 8.500E−02 |
| 78954 | SP140 | chr2 | + | 230245862 | 230245940 | 230244987 | 230245080 | 230247915 | 230248065 | 4.79E−04 | 3.20E−02 | 8.500E−02 |
| 158982 | STAMBP | chr2 | + | 73870142 | 73870352 | 73862202 | 73862304 | 73873351 | 73873659 | 8.16E−08 | 3.02E−05 | 8.500E−02 |
| 91145 | UBXN8 | chr8 | + | 30754664 | 30754749 | 30753034 | 30753105 | 30756764 | 30756887 | 2.76E−09 | 1.71E−06 | 8.600E−02 |
| 110796 | CHROMR | chr2 | + | 178426365 | 178426505 | 178413944 | 178414113 | 178430694 | 178430889 | 2.06E−04 | 1.65E−02 | 8.600E−02 |
| 126708 | FANCG | chr9 | − | 35076970 | 35077101 | 35076431 | 35076583 | 35077263 | 35077399 | 3.72E−09 | 2.21E−06 | 8.600E−02 |
| 139012 | FGR | chr1 | − | 27623690 | 27623929 | 27621558 | 27621657 | 27635064 | 27635185 | 1.02E−06 | 2.38E−04 | 8.600E−02 |
| 180787 | DPEP2 | chr16 | − | 67992063 | 67992193 | 67990046 | 67990131 | 68000447 | 68000586 | 4.27E−05 | 4.87E−03 | 8.600E−02 |
| 14497 | RALGAPB | chr20 | + | 38535073 | 38535207 | 38532729 | 38532859 | 38539775 | 38539958 | 1.90E−05 | 2.56E−03 | 8.700E−02 |
| 88985 | C1orf162 | chr1 | + | 111477333 | 111477428 | 111473791 | 111474030 | 111477982 | 111478472 | 5.66E−05 | 6.05E−03 | 8.700E−02 |
| 184887 | GOLGA2P5 | chr12 | − | 100168451 | 100168633 | 100167549 | 100167639 | 100170564 | 100170667 | 8.10E−11 | 8.95E−08 | 8.700E−02 |
| 195126 | GMFG | chr19 | − | 39335434 | 39335554 | 39329543 | 39329626 | 39335973 | 39336028 | 1.95E−04 | 1.59E−02 | 8.700E−02 |
| 19485 | AC147651.1 | chr7 | + | 524079 | 524280 | 522541 | 522703 | 524853 | 525232 | 3.44E−05 | 4.11E−03 | 8.800E−02 |
| 73782 | IQCB1 | chr3 | − | 121788283 | 121788432 | 121781742 | 121781874 | 121790072 | 121790215 | 1.08E−05 | 1.61E−03 | 8.800E−02 |
| 139897 | ERLIN1 | chr10 | − | 100167347 | 100167406 | 100156144 | 100156234 | 100174207 | 100174281 | 7.06E−09 | 3.89E−06 | 8.800E−02 |
| 29002 | ATOX1 | chr5 | − | 151751703 | 151751779 | 151742821 | 151742959 | 151758545 | 151758631 | 7.38E−06 | 1.17E−03 | 8.900E−02 |
| 122105 | RIDA | chr8 | − | 98108645 | 98108713 | 98106271 | 98106326 | 98117031 | 98117159 | 4.20E−04 | 2.91E−02 | 8.900E−02 |
| 174427 | TSPAN5 | chr4 | − | 98484398 | 98484598 | 98482100 | 98482175 | 98486737 | 98486884 | 2.50E−05 | 3.17E−03 | 8.900E−02 |
| 8381 | SNHG17 | chr20 | − | 38422095 | 38422241 | 38421005 | 38421098 | 38426418 | 38426503 | 8.25E−04 | 4.94E−02 | 9.000E−02 |
| 49462 | MVK | chr12 | + | 109581394 | 109581550 | 109575997 | 109576145 | 109586021 | 109586096 | 6.14E−04 | 3.91E−02 | 9.000E−02 |
| 180190 | PSME3IP1 | chr16 | − | 57178501 | 57178852 | 57174619 | 57174688 | 57186030 | 57186064 | 2.40E−05 | 3.08E−03 | 9.000E−02 |
| 120069 | POLK | chr5 | + | 75593877 | 75594049 | 75587025 | 75587058 | 75596221 | 75597178 | 3.06E−10 | 2.63E−07 | 9.100E−02 |
| 170159 | WDR20 | chr14 | + | 102208602 | 102209862 | 102194937 | 102195120 | 102222829 | 102223667 | 1.04E−04 | 9.74E−03 | 9.100E−02 |
| 171729 | DDX31 | chr9 | − | 132625663 | 132625745 | 132612086 | 132612255 | 132630263 | 132630403 | 2.64E−07 | 7.76E−05 | 9.200E−02 |
| 11193 | POLD1 | chr19 | + | 50395024 | 50395150 | 50384341 | 50384390 | 50398850 | 50399053 | 5.69E−06 | 9.41E−04 | 9.300E−02 |
| 32200 | CAST | chr5 | + | 96726753 | 96726859 | 96695835 | 96695907 | 96727488 | 96727530 | 4.24E−04 | 2.92E−02 | 9.300E−02 |
| 147991 | TM7SF2 | chr11 | + | 65115313 | 65115390 | 65114712 | 65115081 | 65115475 | 65115598 | 5.93E−04 | 3.80E−02 | 9.300E−02 |
| 184533 | NSRP1 | chr17 | + | 30118079 | 30118173 | 30116780 | 30116863 | 30172541 | 30172598 | 4.69E−04 | 3.16E−02 | 9.300E−02 |
| 189708 | FAM210A | chr18 | − | 13671861 | 13671973 | 13663346 | 13666713 | 13726328 | 13726558 | 6.79E−05 | 6.99E−03 | 9.300E−02 |
| 48454 | MYL5 | chr4 | + | 677921 | 678029 | 676072 | 676209 | 678657 | 678765 | 5.32E−07 | 1.40E−04 | 9.400E−02 |
| 75823 | INO800 | chr18 | − | 35487385 | 35487823 | 35480455 | 35480563 | 35489331 | 35489385 | 2.87E−11 | 3.64E−08 | 9.400E−02 |
| 124049 | SIMC1 | chr5 | + | 176289653 | 176290955 | 176238423 | 176238637 | 176295029 | 176295262 | 3.16E−05 | 3.84E−03 | 9.400E−02 |
| 34364 | ABHD16A | chr6 | − | 31702073 | 31702130 | 31700941 | 31701028 | 31702611 | 31702984 | 5.24E−04 | 3.44E−02 | 9.500E−02 |
| 144968 | DAP3 | chr1 | + | 155721516 | 155721618 | 155709772 | 155709824 | 155727607 | 155727738 | 2.29E−14 | 6.82E−11 | 9.500E−02 |
| 84290 | ANKRD26 | chr10 | − | 27005622 | 27005723 | 26995040 | 26995147 | 27006916 | 27006962 | 3.57E−05 | 4.25E−03 | 9.600E−02 |
| 90806 | ADCY10P1 | chr6 | + | 41118128 | 41118184 | 41117475 | 41117665 | 41120522 | 41120657 | 3.52E−04 | 2.53E−02 | 9.600E−02 |
| 184672 | ADAP2 | chr17 | + | 30926826 | 30926918 | 30922939 | 30923070 | 30931888 | 30931968 | 2.43E−04 | 1.87E−02 | 9.600E−02 |
| 39330 | RPL5 | chr1 | + | 92833544 | 92833660 | 92832012 | 92832117 | 92836189 | 92836354 | 2.35E−12 | 4.14E−09 | 9.700E−02 |
| 45752 | ZCWPW1 | chr7 | − | 100408538 | 100408659 | 100406693 | 100406798 | 100409427 | 100409544 | 3.34E−12 | 5.64E−09 | 9.700E−02 |
| 101749 | SUSD3 | chr9 | + | 93077845 | 93077993 | 93058778 | 93058830 | 93084536 | 93085132 | 1.05E−05 | 1.57E−03 | 9.700E−02 |
| 110802 | CHROMR | chr2 | + | 178426365 | 178426680 | 178413676 | 178414113 | 178430694 | 178430889 | 4.04E−04 | 2.82E−02 | 9.700E−02 |
| 150386 | TMEM126A | chr11 | + | 85650248 | 85650341 | 85647966 | 85648089 | 85655593 | 85655708 | 6.70E−05 | 6.93E−03 | 9.700E−02 |
| 181346 | ZFP1 | chr16 | + | 75166769 | 75166913 | 75152908 | 75152966 | 75169252 | 75169326 | 8.78E−08 | 3.22E−05 | 9.700E−02 |
| 142911 | ZNF195 | chr11 | − | 3369356 | 3369502 | 3361808 | 3361889 | 3370974 | 3371070 | 2.18E−04 | 1.72E−02 | 9.900E−02 |
| 11075 | FPR2 | chr19 | + | 51763361 | 51763517 | 51762416 | 51762535 | 51768644 | 51770013 | 2.85E−07 | 8.23E−05 | 1.000E−01 |
| 45589 | DCLRE1C | chr10 | − | 14936537 | 14936593 | 14935462 | 14935564 | 14939809 | 14939869 | 1.62E−07 | 5.37E−05 | 1.000E−01 |
| 114118 | POU6F1 | chr12 | − | 51206788 | 51206883 | 51204172 | 51204368 | 51217641 | 51217667 | 3.13E−04 | 2.31E−02 | 1.000E−01 |
| 21423 | RPF2 | chr6 | + | 110988987 | 110989065 | 110985005 | 110985138 | 110991746 | 110991786 | 1.79E−04 | 1.49E−02 | 1.020E−01 |
| 29794 | MTSS1 | chr8 | − | 124557680 | 124557875 | 124556260 | 124556354 | 124562781 | 124562992 | 1.95E−04 | 1.59E−02 | 1.020E−01 |
| 110658 | TOP1MT | chr8 | − | 143334107 | 143334285 | 143331223 | 143331339 | 143334739 | 143334795 | 5.37E−11 | 6.16E−08 | 1.020E−01 |
| 116598 | CPVL | chr7 | − | 29071772 | 29071904 | 29030576 | 29030759 | 29086483 | 29086550 | 6.61E−07 | 1.67E−04 | 1.020E−01 |
| 132162 | PTPA | chr9 | + | 129127963 | 129128068 | 129123051 | 129123138 | 129131521 | 129131639 | 7.41E−05 | 7.51E−03 | 1.020E−01 |
| 9897 | PANK2 | chr20 | + | 3916926 | 3917050 | 3910576 | 3910830 | 3923243 | 3923631 | 2.84E−04 | 2.13E−02 | 1.040E−01 |
| 147060 | RGS18 | chr1 | + | 192161452 | 192161487 | 192160377 | 192160439 | 192184296 | 192185815 | 3.98E−12 | 6.55E−09 | 1.040E−01 |
| 91323 | CKLF | chr16 | + | 66563071 | 66563217 | 66552562 | 66552793 | 66566082 | 66566251 | 6.03E−04 | 3.84E−02 | 1.050E−01 |
| 149906 | PAK1 | chr11 | − | 77397026 | 77397093 | 77392330 | 77392538 | 77411773 | 77411944 | 2.40E−05 | 3.08E−03 | 1.050E−01 |
| 183107 | HM13 | chr20 | + | 31548676 | 31548793 | 31547381 | 31547606 | 31549028 | 31549114 | 8.18E−08 | 3.02E−05 | 1.060E−01 |
| 71865 | LINC01934 | chr2 | + | 181354207 | 181354343 | 181226470 | 181226539 | 181362686 | 181362902 | 1.49E−06 | 3.19E−04 | 1.070E−01 |
| 21253 | BBOF1 | chr14 | + | 74049701 | 74050195 | 74040564 | 74040645 | 74055583 | 74055685 | 1.99E−06 | 4.05E−04 | 1.080E−01 |
| 106110 | BMPR2 | chr2 | + | 202555251 | 202556531 | 202552715 | 202552888 | 202559695 | 202560341 | 0.00E+00 | 0.00E+00 | 1.080E−01 |
| 96685 | AF117829.1 | chr8 | − | 89725517 | 89725641 | 89724018 | 89724966 | 89757044 | 89757711 | 6.29E−05 | 6.58E−03 | 1.090E−01 |
| 46410 | THTPA | chr14 | + | 23556742 | 23557304 | 23556006 | 23556343 | 23558694 | 23559574 | 2.33E−04 | 1.81E−02 | 1.100E−01 |
| 52673 | C11orf80 | chr11 | + | 66762078 | 66762450 | 66759042 | 66759096 | 66788159 | 66788269 | 6.44E−04 | 4.05E−02 | 1.100E−01 |
| 73231 | UBL7 | chr15 | − | 74451435 | 74451520 | 74445976 | 74446227 | 74456551 | 74456671 | 4.13E−05 | 4.77E−03 | 1.100E−01 |
| 143408 | TRIM5 | chr11 | − | 5665168 | 5665395 | 5664057 | 5664173 | 5665655 | 5665682 | 8.80E−05 | 8.61E−03 | 1.100E−01 |
| 147626 | DICER1-AS1 | chr14 | + | 95158875 | 95159035 | 95157918 | 95158234 | 95179503 | 95179925 | 5.53E−08 | 2.20E−05 | 1.100E−01 |
| 180955 | AC009022.1 | chr16 | − | 69990596 | 69990683 | 69986380 | 69986509 | 69996068 | 69996226 | 5.88E−06 | 9.67E−04 | 1.100E−01 |
| 15553 | POLR2J3 | chr7 | − | 102545302 | 102545420 | 102544705 | 102544776 | 102566996 | 102567083 | 4.56E−05 | 5.15E−03 | 1.110E−01 |
| 116625 | MATR3 | chr5 | + | 139279934 | 139280058 | 139279050 | 139279129 | 139307238 | 139307556 | 1.83E−07 | 5.90E−05 | 1.110E−01 |
| 127991 | CBWD3 | chr9 | + | 68269599 | 68269722 | 68268933 | 68268978 | 68285988 | 68286040 | 2.16E−12 | 3.83E−09 | 1.120E−01 |
| 114119 | POU6F1 | chr12 | − | 51206788 | 51206947 | 51204320 | 51204368 | 51217641 | 51217708 | 2.80E−05 | 3.47E−03 | 1.130E−01 |
| 137474 | TYSND1 | chr10 | − | 70143841 | 70143972 | 70137980 | 70140141 | 70145420 | 70146700 | 9.39E−06 | 1.42E−03 | 1.130E−01 |
| 21255 | BBOF1 | chr14 | + | 74049701 | 74050195 | 74047929 | 74048074 | 74055583 | 74055685 | 8.37E−11 | 9.09E−08 | 1.140E−01 |
| 185888 | NBR2 | chr17 | + | 43132598 | 43132723 | 43131207 | 43131270 | 43138656 | 43138922 | 7.30E−07 | 1.82E−04 | 1.140E−01 |
| 18238 | CCM2 | chr7 | + | 45071749 | 45071872 | 45000299 | 45000363 | 45072725 | 45072786 | 5.48E−04 | 3.55E−02 | 1.150E−01 |
| 26610 | MAP3K8 | chr10 | + | 30437175 | 30437406 | 30434183 | 30434378 | 30438915 | 30439274 | 4.96E−05 | 5.48E−03 | 1.170E−01 |
| 52233 | AC008035.1 | chr12 | + | 46571255 | 46571412 | 46537501 | 46537631 | 46652389 | 46652550 | 5.89E−07 | 1.52E−04 | 1.170E−01 |
| 189697 | LDLRAD4 | chr18 | + | 13484036 | 13484119 | 13465014 | 13465111 | 13621116 | 13621270 | 7.16E−04 | 4.40E−02 | 1.170E−01 |
| 36507 | PSMG4 | chr6 | + | 3264208 | 3264325 | 3263683 | 3263759 | 3267590 | 3267781 | 5.52E−13 | 1.15E−09 | 1.190E−01 |
| 102095 | ANAPC10 | chr4 | − | 144999256 | 144999294 | 144995160 | 144995603 | 145081659 | 145081750 | 5.39E−04 | 3.51E−02 | 1.200E−01 |
| 91488 | ZMYM1 | chr1 | + | 35095818 | 35095891 | 35093913 | 35094083 | 35097316 | 35097478 | 1.62E−04 | 1.38E−02 | 1.220E−01 |
| 90861 | ADCY10P1 | chr6 | + | 41138608 | 41138726 | 41137356 | 41137561 | 41139855 | 41140069 | 3.48E−05 | 4.14E−03 | 1.240E−01 |
| 6980 | TCFL5 | chr20 | − | 62859363 | 62859526 | 62854015 | 62854157 | 62860124 | 62860308 | 2.67E−07 | 7.83E−05 | 1.260E−01 |
| 23439 | POLL | chr10 | − | 101585315 | 101585478 | 101582762 | 101582891 | 101587245 | 101587382 | 1.55E−04 | 1.33E−02 | 1.270E−01 |
| 153428 | DPH7 | chr9 | − | 137576308 | 137576402 | 137575708 | 137576167 | 137578624 | 137578890 | 3.40E−04 | 2.45E−02 | 1.270E−01 |
| 124336 | MOK | chr14 | − | 102231706 | 102231821 | 102226139 | 102226408 | 102232534 | 102232671 | 2.84E−08 | 1.28E−05 | 1.280E−01 |
| 223 | MECP2 | chrX | − | 154056940 | 154057254 | 154032206 | 154032557 | 154097603 | 154097737 | 5.80E−04 | 3.73E−02 | 1.290E−01 |
| 119208 | HLA-DPA1 | chr6 | − | 33069018 | 33069300 | 33064568 | 33065347 | 33073470 | 33073669 | 8.76E−09 | 4.64E−06 | 1.310E−01 |
| 183106 | HM13 | chr20 | + | 31548379 | 31548793 | 31547464 | 31547606 | 31549028 | 31549114 | 5.44E−10 | 4.24E−07 | 1.310E−01 |
| 49461 | MVK | chr12 | + | 109579801 | 109579946 | 109575997 | 109576145 | 109586021 | 109586125 | 5.42E−05 | 5.85E−03 | 1.330E−01 |
| 146249 | ZBTB8OS | chr1 | − | 32634767 | 32634792 | 32627507 | 32627544 | 32650401 | 32650525 | 6.92E−04 | 4.28E−02 | 1.330E−01 |
| 28412 | DOCK2 | chr5 | + | 170075946 | 170076084 | 170067509 | 170067686 | 170078974 | 170079146 | 1.42E−07 | 4.85E−05 | 1.360E−01 |
| 29209 | WBP1 | chr2 | + | 74458831 | 74459117 | 74458471 | 74458671 | 74459477 | 74459562 | 5.28E−05 | 5.73E−03 | 1.370E−01 |
| 98914 | VMP1 | chr17 | + | 59764970 | 59765138 | 59707647 | 59707748 | 59808795 | 59808876 | 1.93E−06 | 3.97E−04 | 1.380E−01 |
| 97903 | MYOM1 | chr18 | − | 3135546 | 3135730 | 3134649 | 3134824 | 3141938 | 3142063 | 2.96E−07 | 8.46E−05 | 1.390E−01 |
| 60922 | FYB1 | chr5 | − | 39212677 | 39213017 | 39202773 | 39202987 | 39274402 | 39274528 | 2.40E−04 | 1.85E−02 | 1.400E−01 |
| 104389 | EVA1C | chr21 | + | 32457596 | 32457720 | 32412436 | 32413013 | 32467695 | 32467848 | 7.46E−04 | 4.55E−02 | 1.460E−01 |
| 41045 | AC093157.1 | chr1 | + | 101072659 | 101072830 | 101025868 | 101026061 | 101077362 | 101077508 | 3.90E−04 | 2.75E−02 | 1.480E−01 |
| 110023 | TRAF3IP1 | chr2 | + | 238333959 | 238334035 | 238328925 | 238329342 | 238347454 | 238347475 | 1.10E−05 | 1.62E−03 | 1.490E−01 |
| 8378 | SNHG17 | chr20 | − | 38422091 | 38422241 | 38421005 | 38421098 | 38426418 | 38426503 | 1.43E−05 | 2.02E−03 | 1.510E−01 |
| 25604 | YWHAH | chr22 | + | 31945499 | 31945678 | 31944965 | 31945235 | 31956138 | 31956482 | 2.00E−04 | 1.62E−02 | 1.510E−01 |
| 1050 | ARMCX5-GPRASP2 | chrX | + | 102639399 | 102639533 | 102605481 | 102605653 | 102713781 | 102713849 | 0.00E+00 | 0.00E+00 | 1.520E−01 |
| 180957 | AC009022.1 | chr16 | − | 69992910 | 69993248 | 69986380 | 69986509 | 69996068 | 69996226 | 8.12E−05 | 8.07E−03 | 1.520E−01 |
| 129952 | ZNF232 | chr17 | − | 5111075 | 5111219 | 5108925 | 5109868 | 5111799 | 5111815 | 3.35E−04 | 2.43E−02 | 1.540E−01 |
| 38545 | CENPN | chr16 | + | 81022596 | 81022652 | 81020099 | 81020276 | 81022754 | 81022885 | 6.02E−04 | 3.84E−02 | 1.550E−01 |
| 29206 | WBP1 | chr2 | + | 74458831 | 74458919 | 74458438 | 74458671 | 74459477 | 74459562 | 6.08E−05 | 6.42E−03 | 1.570E−01 |
| 84489 | ITGB3BP | chr1 | − | 63510064 | 63510181 | 63508527 | 63508570 | 63529130 | 63529186 | 2.53E−10 | 2.26E−07 | 1.570E−01 |
| 161267 | MPV17 | chr2 | − | 27317077 | 27317234 | 27312993 | 27313154 | 27322447 | 27322522 | 2.53E−08 | 1.17E−05 | 1.570E−01 |
| 39862 | MTIF3 | chr13 | − | 27441016 | 27441077 | 27437115 | 27437273 | 27445087 | 27445137 | 2.66E−15 | 1.04E−11 | 1.600E−01 |
| 81387 | LRRC23 | chr12 | + | 6909889 | 6910026 | 6907314 | 6907445 | 6913890 | 6914228 | 2.34E−04 | 1.82E−02 | 1.600E−01 |
| 196128 | TMEM143 | chr19 | − | 48360071 | 48360176 | 48345159 | 48345354 | 48363290 | 48363531 | 1.29E−06 | 2.85E−04 | 1.600E−01 |
| 14423 | NEIL2 | chr8 | + | 11770173 | 11770335 | 11769698 | 11769791 | 11771445 | 11771585 | 7.16E−04 | 4.40E−02 | 1.680E−01 |
| 62383 | TMEM161B-AS1 | chr5 | + | 88283009 | 88283086 | 88282714 | 88282866 | 88287438 | 88287616 | 7.14E−06 | 1.14E−03 | 1.680E−01 |
| 38810 | INTS7 | chr1 | − | 211946606 | 211946645 | 211944783 | 211944969 | 211952568 | 211952701 | 2.67E−04 | 2.02E−02 | 1.700E−01 |
| 113347 | TRAF3IP2-AS1 | chr6 | + | 111574620 | 111574802 | 111483540 | 111483665 | 111576361 | 111576470 | 5.57E−04 | 3.61E−02 | 1.740E−01 |
| 171865 | GOLGA8A | chr15 | − | 34435382 | 34435471 | 34407523 | 34407735 | 34437397 | 34437466 | 1.22E−04 | 1.11E−02 | 1.740E−01 |
| 82949 | BBS9 | chr7 | + | 33352858 | 33352873 | 33349067 | 33349170 | 33357854 | 33357995 | 9.71E−06 | 1.47E−03 | 1.750E−01 |
| 134251 | PPIEL | chr1 | − | 39523806 | 39523896 | 39522279 | 39522505 | 39524835 | 39525057 | 5.07E−04 | 3.36E−02 | 1.770E−01 |
| 172725 | TUBGCP4 | chr15 | + | 43399111 | 43399174 | 43398037 | 43398179 | 43400043 | 43400221 | 3.59E−05 | 4.26E−03 | 1.770E−01 |
| 15753 | AC060780.1 | chr17 | − | 43159422 | 43159727 | 43148367 | 43148783 | 43166979 | 43167849 | 5.18E−04 | 3.41E−02 | 1.800E−01 |
| 37036 | ECHDC2 | chr1 | − | 52899173 | 52899224 | 52896222 | 52896597 | 52911565 | 52911655 | 1.41E−06 | 3.07E−04 | 1.820E−01 |
| 141611 | UROS | chr10 | − | 125789282 | 125789363 | 125788585 | 125789005 | 125794021 | 125794388 | 4.35E−04 | 2.98E−02 | 1.820E−01 |
| 29621 | MAFG | chr17 | − | 81923352 | 81923416 | 81923149 | 81923214 | 81927527 | 81927708 | 6.25E−07 | 1.59E−04 | 1.860E−01 |
| 195898 | GIPR | chr19 | + | 45671284 | 45671392 | 45670634 | 45670734 | 45672850 | 45672954 | 3.11E−04 | 2.30E−02 | 1.860E−01 |
| 26863 | HCLS1 | chr3 | − | 121636433 | 121636489 | 121634206 | 121634418 | 121642926 | 121642981 | 0.00E+00 | 0.00E+00 | 1.910E−01 |
| 108516 | AC016727.1 | chr2 | + | 61480299 | 61480583 | 61471355 | 61471379 | 61482626 | 61483913 | 2.18E−04 | 1.72E−02 | 1.920E−01 |
| 8377 | SNHG17 | chr20 | − | 38422091 | 38422241 | 38421005 | 38421098 | 38425934 | 38426048 | 3.34E−04 | 2.43E−02 | 1.950E−01 |
| 190800 | AC022137.3 | chr19 | + | 53446226 | 53446337 | 53431983 | 53432028 | 53447195 | 53447283 | 2.01E−04 | 1.63E−02 | 1.950E−01 |
| 139974 | CWF19L1 | chr10 | − | 100260217 | 100260319 | 100253420 | 100253539 | 100267570 | 100267638 | 2.33E−04 | 1.81E−02 | 2.010E−01 |
| 81388 | LRRC23 | chr12 | + | 6912729 | 6913027 | 6907314 | 6907445 | 6913890 | 6914194 | 7.97E−06 | 1.24E−03 | 2.080E−01 |
| 196129 | TMEM143 | chr19 | − | 48360071 | 48360183 | 48345159 | 48345354 | 48363290 | 48363674 | 3.32E−05 | 3.99E−03 | 2.090E−01 |
| 22804 | VNN2 | chr6 | − | 132760631 | 132760803 | 132757670 | 132757902 | 132763382 | 132763431 | 3.87E−05 | 4.52E−03 | 2.140E−01 |
| 149954 | AAMDC | chr11 | + | 77841018 | 77841060 | 77821186 | 77821237 | 77842478 | 77842628 | 4.39E−07 | 1.18E−04 | 2.200E−01 |
| 50604 | TPRKB | chr2 | − | 73734527 | 73734588 | 73732162 | 73732285 | 73737301 | 73737386 | 4.40E−05 | 5.00E−03 | 2.280E−01 |
| 29571 | FCHSD1 | chr5 | − | 141642396 | 141642503 | 141641701 | 141641757 | 141644217 | 141644438 | 2.48E−06 | 4.83E−04 | 2.460E−01 |
| 139815 | PYROXD2 | chr10 | − | 98388353 | 98388508 | 98387200 | 98387307 | 98391009 | 98391082 | 1.09E−04 | 1.01E−02 | 2.490E−01 |
| 177507 | STRA6LP | chr9 | + | 97347313 | 97347666 | 97343339 | 97343563 | 97350408 | 97350555 | 1.73E−08 | 8.32E−06 | 2.550E−01 |
| 27105 | SERPINB9P1 | chr6 | − | 2857910 | 2857991 | 2854657 | 2855690 | 2876292 | 2876425 | 4.59E−04 | 3.11E−02 | 2.660E−01 |
| 72653 | AC245100.4 | chr1 | + | 148424062 | 148424189 | 148420371 | 148420607 | 148424626 | 148424638 | 3.71E−07 | 1.02E−04 | 2.680E−01 |
| 171084 | PGAP3 | chr17 | − | 39676613 | 39676755 | 39674616 | 39674679 | 39685921 | 39686019 | 2.22E−16 | 1.00E−12 | 3.190E−01 |

TABLE 16
MXE_WBC_FXSvsTD

| | | | | 1stExon | | 2ndExon | | upstream | | downstream | | | | IncLevel |
|---|---|---|---|---|---|---|---|---|---|---|---|---|---|---|
| ID | GeneID | chr | strand | Start_0base | End | Start_0base | End | ES | EE | ES | EE | PValue | FDR | Difference |
| 18883 | CR1 | chr1 | + | 207526752 | 207526929 | 207545307 | 207545484 | 207523610 | 207524009 | 207545575 | 207545678 | 5.48E−12 | 7.24E−09 | 0.438 |
| 42190 | PIGL | chr17 | + | 16313546 | 16313614 | 16316680 | 16316712 | 16299887 | 16299978 | 16317774 | 16317908 | 9.48E−08 | 2.53E−05 | 0.239 |
| 21240 | XPNPEP3 | chr22 | + | 40870047 | 40870139 | 40881769 | 40882177 | 40868998 | 40869115 | 40907586 | 40907649 | 4.88E−04 | 1.96E−02 | 0.238 |
| 13289 | MAPKBP1 | chr15 | + | 41813620 | 41813781 | 41815258 | 41815405 | 41812918 | 41813101 | 41815623 | 41815799 | 4.39E−05 | 3.28E−03 | 0.231 |
| 32138 | TBC1D12 | chr10 | + | 94522343 | 94522453 | 94531201 | 94531460 | 94521954 | 94522083 | 94533027 | 94536332 | 2.10E−04 | 1.07E−02 | 0.188 |
| 4152 | AC004593.2 | chr7 | + | 29146848 | 29146960 | 29354624 | 29354663 | 29145853 | 29146745 | 29367931 | 29367987 | 7.06E−04 | 2.51E−02 | 0.184 |
| 9042 | AC016831.6 | chr7 | − | 130912109 | 130912152 | 130945719 | 130945835 | 130884316 | 130884396 | 131052424 | 131052555 | 4.24E−06 | 5.38E−04 | 0.184 |
| 3177 | WDR60 | chr7 | + | 158905988 | 158906091 | 158911549 | 158911679 | 158902375 | 158902595 | 158912984 | 158913096 | 1.03E−03 | 3.19E−02 | 0.179 |
| 5157 | ZNF720 | chr16 | + | 31723257 | 31723353 | 31753765 | 31753900 | 31722625 | 31722752 | 31759356 | 31761565 | 2.19E−04 | 1.10E−02 | 0.171 |
| 41158 | MYO15B | chr17 | + | 75603031 | 75603077 | 75605863 | 75606021 | 75602829 | 75602945 | 75610165 | 75610259 | 1.69E−04 | 9.09E−03 | 0.167 |
| 29758 | HSD17B3 | chr9 | − | 96252802 | 96252910 | 96254867 | 96254943 | 96251417 | 96251485 | 96298415 | 96298462 | 1.56E−03 | 4.25E−02 | 0.165 |
| 40789 | WHAMMP3 | chr15 | + | 22674420 | 22674586 | 22676804 | 22676934 | 22672689 | 22672859 | 22678196 | 22678383 | 9.46E−04 | 3.02E−02 | 0.164 |
| 16517 | AL392172.1 | chr1 | + | 222835118 | 222835215 | 222836995 | 222837066 | 222827427 | 222827601 | 222837218 | 222837229 | 3.30E−05 | 2.62E−03 | 0.163 |
| 36990 | ATXN2 | chr12 | − | 111456028 | 111456256 | 111464661 | 111464715 | 111452213 | 111452840 | 111470107 | 111470240 | 2.59E−04 | 1.24E−02 | 0.152 |
| 16588 | AL356481.1 | chr9 | − | 128536826 | 128536955 | 128543378 | 128543684 | 128528656 | 128529000 | 128552367 | 128552410 | 5.50E−04 | 2.13E−02 | 0.149 |
| 40376 | PDE8A | chr15 | + | 85109052 | 85109130 | 85113376 | 85113447 | 85100155 | 85100198 | 85113872 | 85113956 | 4.81E−04 | 1.94E−02 | 0.148 |
| 44077 | ZNF266 | chr19 | − | 9433667 | 9433786 | 9434797 | 9434859 | 9420064 | 9420218 | 9435107 | 9435193 | 1.78E−03 | 4.65E−02 | 0.148 |
| 21072 | AL162258.1 | chr1 | − | 153627482 | 153627598 | 153631010 | 153631078 | 153626331 | 153626533 | 153632033 | 153632063 | 3.47E−05 | 2.73E−03 | 0.146 |
| 41945 | CAMKK1 | chr17 | − | 3882527 | 3882564 | 3883041 | 3883175 | 3881626 | 3881648 | 3883428 | 3883480 | 1.24E−04 | 7.17E−03 | 0.145 |
| 34393 | PPFIA1 | chr11 | + | 70349922 | 70350039 | 70350993 | 70351023 | 70348188 | 70348420 | 70354300 | 70354452 | 1.48E−03 | 4.13E−02 | 0.143 |
| 44079 | ZNF266 | chr19 | − | 9434074 | 9434235 | 9434797 | 9434859 | 9433667 | 9433786 | 9435107 | 9435193 | 5.54E−04 | 2.13E−02 | 0.143 |
| 5917 | CFAP70 | chr10 | − | 73277239 | 73277361 | 73278178 | 73278337 | 73275445 | 73275598 | 73291225 | 73291444 | 2.60E−04 | 1.25E−02 | 0.139 |
| 33519 | ACCS | chr11 | + | 44071255 | 44071315 | 44073446 | 44073517 | 44067627 | 44067915 | 44074611 | 44074681 | 5.82E−04 | 2.20E−02 | 0.138 |
| 20632 | ATF7IP2 | chr16 | + | 10473922 | 10473989 | 10480878 | 10480964 | 10473478 | 10473534 | 10481835 | 10483637 | 7.24E−05 | 4.76E−03 | 0.137 |
| 3336 | NEIL2 | chr8 | + | 11771445 | 11771590 | 11779597 | 11779950 | 11770173 | 11770335 | 11783202 | 11783399 | 6.53E−04 | 2.40E−02 | 0.136 |
| 1571 | TAF4 | chr20 | − | 62006509 | 62006758 | 62007546 | 62007636 | 62003730 | 62003878 | 62009051 | 62009174 | 4.42E−08 | 1.38E−05 | 0.129 |
| 4975 | PNPT1 | chr2 | − | 55666990 | 55667093 | 55671318 | 55671376 | 55661955 | 55662026 | 55671994 | 55672046 | 2.52E−04 | 1.22E−02 | 0.128 |
| 35502 | HLA-DRB5 | chr6 | − | 32519369 | 32519651 | 32521904 | 32522174 | 32518555 | 32518666 | 32530124 | 32530287 | 9.51E−04 | 3.02E−02 | 0.128 |
| 6165 | DDX60L | chr4 | − | 168391539 | 168391644 | 168395958 | 168396124 | 168384611 | 168384812 | 168400825 | 168400978 | 1.55E−03 | 4.25E−02 | 0.126 |
| 35988 | YAF2 | chr12 | − | 42172139 | 42172209 | 42235613 | 42235755 | 42161612 | 42161765 | 42237598 | 42237724 | 3.91E−04 | 1.66E−02 | 0.126 |
| 18038 | SP140 | chr2 | + | 230245862 | 230245940 | 230247915 | 230248065 | 230244987 | 230245080 | 230255451 | 230255532 | 1.12E−03 | 3.39E−02 | 0.125 |
| 21535 | KIAA1841 | chr2 | + | 61073457 | 61073551 | 61076919 | 61077158 | 61071605 | 61071792 | 61081451 | 61081510 | 1.25E−03 | 3.67E−02 | 0.125 |
| 32881 | TNNT3 | chr11 | + | 1926469 | 1926481 | 1929809 | 1929828 | 1925255 | 1925276 | 1932468 | 1932514 | 1.22E−05 | 1.24E−03 | 0.125 |
| 8747 | ARFIP1 | chr4 | + | 152807203 | 152807248 | 152809672 | 152809733 | 152779992 | 152780091 | 152810079 | 152810388 | 1.72E−03 | 4.55E−02 | 0.12 |
| 19983 | TUBD1 | chr17 | − | 59866608 | 59866749 | 59874538 | 59874703 | 59863663 | 59863847 | 59878102 | 59878334 | 1.54E−03 | 4.24E−02 | 0.12 |
| 24955 | POMZP3 | chr7 | − | 76611453 | 76611591 | 76618182 | 76618300 | 76609985 | 76610215 | 76625521 | 76625683 | 1.02E−03 | 3.18E−02 | 0.117 |
| 28807 | SH3TC1 | chr4 | + | 8225174 | 8225216 | 8226979 | 8228644 | 8222839 | 8222970 | 8231975 | 8232156 | 1.21E−05 | 1.23E−03 | 0.117 |
| 4055 | POLR2J4 | chr7 | − | 43986600 | 43986677 | 44013592 | 44013679 | 43973209 | 43973323 | 44014605 | 44014783 | 1.05E−04 | 6.36E−03 | 0.116 |
| 7066 | CDKL3 | chr5 | − | 134308573 | 134308727 | 134312291 | 134312380 | 134308137 | 134308466 | 134319357 | 134319497 | 8.96E−04 | 2.91E−02 | 0.112 |
| 43224 | CD300LF | chr17 | − | 74696194 | 74696217 | 74698368 | 74698481 | 74695724 | 74695859 | 74704477 | 74704816 | 1.70E−03 | 4.51E−02 | 0.112 |
| 12681 | ELP1 | chr9 | − | 108918810 | 108918901 | 108919252 | 108919349 | 108917546 | 108917670 | 108922841 | 108922927 | 1.19E−03 | 3.55E−02 | 0.111 |
| 26852 | RPTOR | chr17 | + | 80707840 | 80707999 | 80730559 | 80730706 | 80643727 | 80643810 | 80754009 | 80754185 | 9.05E−05 | 5.69E−03 | 0.111 |
| 44078 | ZNF266 | chr19 | − | 9434074 | 9434235 | 9434797 | 9434859 | 9420064 | 9420218 | 9435107 | 9435193 | 1.19E−03 | 3.55E−02 | 0.111 |
| 4054 | POLR2J4 | chr7 | − | 43986600 | 43986677 | 43987677 | 43987888 | 43973209 | 43973323 | 43988085 | 43988247 | 1.46E−04 | 8.16E−03 | 0.11 |
| 29298 | CBWD3 | chr9 | + | 68266219 | 68266306 | 68268933 | 68268978 | 68264467 | 68264514 | 68285988 | 68286040 | 1.46E−04 | 8.17E−03 | 0.11 |
| 24477 | WDR12 | chr2 | − | 202894580 | 202894626 | 202897299 | 202897415 | 202892616 | 202892702 | 202899530 | 202899637 | 8.91E−06 | 9.88E−04 | 0.109 |
| 28262 | PVT1 | chr8 | + | 128009589 | 128009718 | 128082752 | 128082848 | 127989161 | 127989291 | 128096517 | 128096654 | 5.56E−05 | 3.96E−03 | 0.109 |
| 30601 | LLGL2 | chr17 | + | 75574212 | 75574260 | 75574609 | 75574668 | 75573951 | 75573980 | 75574873 | 75574906 | 8.52E−05 | 5.46E−03 | 0.107 |
| 1554 | TCFL5 | chr20 | − | 62854015 | 62854157 | 62859363 | 62859526 | 62841004 | 62842097 | 62860124 | 62860308 | 1.97E−04 | 1.02E−02 | 0.104 |
| 13145 | MCTP1 | chr5 | − | 94870416 | 94870491 | 94870871 | 94870973 | 94867221 | 94868452 | 94871314 | 94871417 | 9.96E−06 | 1.08E−03 | 0.104 |
| 13524 | ST3GAL5 | chr2 | − | 85852859 | 85853099 | 85861180 | 85861292 | 85851263 | 85851703 | 85863361 | 85863485 | 1.51E−03 | 4.19E−02 | 0.103 |
| 20964 | UBXN8 | chr8 | + | 30754664 | 30754787 | 30756764 | 30756887 | 30753034 | 30753105 | 30760887 | 30760929 | 4.83E−04 | 1.95E−02 | 0.102 |
| 36995 | ATXN2 | chr12 | − | 111464661 | 111464715 | 111470107 | 111470240 | 111457213 | 111457359 | 111470557 | 111470742 | 3.72E−06 | 4.90E−04 | 0.102 |
| 5766 | MICA | chr6 | + | 31410542 | 31410797 | 31411946 | 31412225 | 31399783 | 31400783 | 31412324 | 31412460 | 5.91E−06 | 7.00E−04 | 0.101 |
| 8399 | CARD8 | chr19 | − | 48242539 | 48242644 | 48246074 | 48246381 | 48240961 | 48241063 | 48249522 | 48249633 | 1.05E−03 | 3.23E−02 | 0.101 |
| 8400 | CARD8 | chr19 | − | 48242539 | 48242644 | 48246074 | 48246381 | 48240961 | 48241063 | 48249750 | 48249847 | 1.72E−03 | 4.55E−02 | 0.101 |
| 13049 | AL121845.3 | chr20 | + | 63735158 | 63735564 | 63737532 | 63737647 | 63734704 | 63734824 | 63737820 | 63737902 | 3.17E−05 | 2.55E−03 | 0.099 |
| 29252 | IL32 | chr16 | + | 3067983 | 3068010 | 3068179 | 3068239 | 3067553 | 3067613 | 3068989 | 3069667 | 9.71E−04 | 3.06E−02 | 0.099 |
| 5626 | GMPPB | chr3 | − | 49722431 | 49722510 | 49722595 | 49722754 | 49722230 | 49722358 | 49722971 | 49723114 | 8.07E−05 | 5.22E−03 | 0.098 |
| 23611 | ZEB2 | chr2 | − | 144403915 | 144404130 | 144424795 | 144424867 | 144401198 | 144401307 | 144429768 | 144430026 | 4.41E−04 | 1.81E−02 | 0.098 |
| 26157 | TBC1D8 | chr2 | − | 101032267 | 101032385 | 101033543 | 101033758 | 101029490 | 101029776 | 101036017 | 101036168 | 4.63E−07 | 9.26E−05 | 0.098 |
| 32352 | LEMD3 | chr12 | + | 65245668 | 65245774 | 65245860 | 65245939 | 65243387 | 65243469 | 65246161 | 65248355 | 4.39E−05 | 3.29E−03 | 0.098 |
| 41376 | SLC38A1 | chr12 | − | 46201097 | 46201198 | 46203009 | 46203089 | 46198624 | 46198743 | 46204300 | 46204417 | 8.29E−04 | 2.78E−02 | 0.098 |
| 22116 | JARID2 | chr6 | + | 15468541 | 15468718 | 15487306 | 15487542 | 15452005 | 15452175 | 15496131 | 15497170 | 5.33E−04 | 2.08E−02 | 0.097 |
| 27861 | SLC38A9 | chr5 | − | 55633694 | 55635657 | 55645788 | 55645895 | 55627890 | 55627980 | 55649206 | 55649314 | 1.03E−05 | 1.11E−03 | 0.096 |
| 33553 | AC119396.1 | chr19 | + | 7362780 | 7362905 | 7372811 | 7373071 | 7348942 | 7349241 | 7375719 | 7375870 | 2.80E−04 | 1.31E−02 | 0.096 |
| 20257 | UBE3C | chr7 | + | 157216866 | 157216971 | 157220688 | 157220776 | 157207702 | 157207935 | 157223253 | 157223351 | 1.32E−07 | 3.29E−05 | 0.095 |
| 22892 | MARCHF8 | chr10 | − | 45461230 | 45461411 | 45489366 | 45489417 | 45459119 | 45459267 | 45533109 | 45533289 | 1.37E−05 | 1.33E−03 | 0.095 |
| 39762 | TUBGCP4 | chr15 | + | 43400043 | 43400221 | 43401715 | 43401850 | 43399111 | 43399174 | 43403682 | 43403799 | 7.37E−04 | 2.57E−02 | 0.095 |
| 17472 | SLAMF1 | chr1 | − | 160624095 | 160624185 | 160634612 | 160634897 | 160623502 | 160623585 | 160637190 | 160637529 | 2.36E−04 | 1.17E−02 | 0.092 |
| 13535 | GTF2H2 | chr5 | − | 71045443 | 71045507 | 71048005 | 71048113 | 71042180 | 71042284 | 71048761 | 71048849 | 1.06E−03 | 3.25E−02 | 0.091 |
| 26386 | IRF8 | chr16 | + | 85913130 | 85913236 | 85918416 | 85918803 | 85911569 | 85911658 | 85920108 | 85920224 | 1.74E−04 | 9.29E−03 | 0.091 |
| 24954 | POMZP3 | chr7 | − | 76611453 | 76611591 | 76611721 | 76611813 | 76609985 | 76610215 | 76618182 | 76618300 | 1.77E−03 | 4.63E−02 | 0.09 |
| 26307 | MCM6 | chr2 | − | 135862606 | 135862748 | 135866131 | 135866277 | 135859300 | 135859442 | 135866562 | 135866728 | 1.12E−03 | 3.39E−02 | 0.09 |
| 28293 | PVT1 | chr8 | + | 128070161 | 128070272 | 128082752 | 128082848 | 127989161 | 127989291 | 128096517 | 128096654 | 2.58E−04 | 1.24E−02 | 0.09 |
| 44471 | NIBAN3 | chr19 | + | 17537375 | 17537543 | 17539149 | 17539265 | 17533586 | 17533701 | 17539346 | 17539451 | 1.45E−03 | 4.07E−02 | 0.09 |
| 1974 | NFS1 | chr20 | − | 35675044 | 35675202 | 35680736 | 35680871 | 35674511 | 35674617 | 35681887 | 35681981 | 5.54E−04 | 2.13E−02 | 0.088 |
| 4465 | KDM3B | chr5 | + | 138381515 | 138381590 | 138391012 | 138392261 | 138379583 | 138379708 | 138393170 | 138393372 | 1.05E−03 | 3.23E−02 | 0.088 |
| 9505 | AC114490.2 | chr1 | − | 34983766 | 34983947 | 34992233 | 34992387 | 34981532 | 34982119 | 35003967 | 35004005 | 2.49E−04 | 1.21E−02 | 0.088 |
| 37856 | ZC3H13 | chr13 | − | 46020448 | 46020557 | 46042163 | 46042275 | 46011414 | 46011554 | 46044954 | 46045064 | 1.58E−03 | 4.28E−02 | 0.088 |
| 8696 | TGS1 | chr8 | + | 55786237 | 55787060 | 55790181 | 55790299 | 55785718 | 55785891 | 55792697 | 55792784 | 9.47E−04 | 3.02E−02 | 0.087 |
| 18887 | CR1 | chr1 | + | 207609289 | 207609688 | 207611676 | 207611853 | 207607250 | 207607336 | 207611938 | 207612041 | 4.31E−05 | 3.24E−03 | 0.087 |
| 24960 | POMZP3 | chr7 | − | 76618182 | 76618300 | 76625521 | 76625683 | 76611721 | 76611813 | 76625999 | 76626215 | 1.23E−03 | 3.64E−02 | 0.087 |
| 24961 | POMZP3 | chr7 | − | 76618182 | 76618615 | 76625521 | 76625683 | 76611721 | 76611813 | 76625999 | 76626127 | 9.30E−04 | 2.98E−02 | 0.087 |
| 31569 | DDX21 | chr10 | + | 68971890 | 68972052 | 68973544 | 68973664 | 68970200 | 68970350 | 68974669 | 68974743 | 9.29E−04 | 2.98E−02 | 0.087 |
| 12291 | SYNE1 | chr6 | − | 152176393 | 152176560 | 152180135 | 152180294 | 152164162 | 152164325 | 152185273 | 152185411 | 2.82E−06 | 3.96E−04 | 0.086 |
| 17689 | ZBTB17 | chr1 | − | 15945714 | 15945840 | 15946934 | 15947123 | 15944936 | 15945202 | 15948290 | 15948497 | 1.70E−04 | 9.14E−03 | 0.086 |
| 22539 | MYOM1 | chr18 | − | 3075724 | 3075761 | 3079178 | 3079342 | 3075453 | 3075476 | 3083788 | 3083894 | 1.76E−03 | 4.61E−02 | 0.085 |
| 31408 | TIMM23B-AGAP6 | chr10 | + | 49987164 | 49987234 | 49988750 | 49988938 | 49973011 | 49973142 | 49989307 | 49989376 | 4.87E−06 | 6.09E−04 | 0.085 |
| 37373 | SBNO1 | chr12 | − | 123315547 | 123315660 | 123317220 | 123317356 | 123315372 | 123315444 | 123319899 | 123320031 | 1.23E−03 | 3.64E−02 | 0.084 |
| 40651 | RAB11FIP3 | chr16 | + | 491133 | 491268 | 496823 | 496859 | 488850 | 489000 | 503003 | 503097 | 8.48E−05 | 5.44E−03 | 0.084 |
| 41486 | CNOT1 | chr16 | − | 58558472 | 58558674 | 58560211 | 58560362 | 58556846 | 58556993 | 58574608 | 58574760 | 1.18E−03 | 3.53E−02 | 0.084 |
| 18318 | NMRK1 | chr9 | − | 75069741 | 75069813 | 75069894 | 75070042 | 75068995 | 75069102 | 75077158 | 75077207 | 5.01E−05 | 3.65E−03 | 0.083 |
| 26387 | IRF8 | chr16 | + | 85914472 | 85914520 | 85918416 | 85918803 | 85913130 | 85913236 | 85920108 | 85920224 | 2.21E−04 | 1.11E−02 | 0.083 |
| 30144 | TSC2 | chr16 | + | 2079275 | 2079428 | 2079556 | 2079669 | 2078238 | 2079196 | 2080164 | 2080377 | 1.92E−03 | 4.88E−02 | 0.083 |
| 44881 | RCHY1 | chr4 | − | 75491888 | 75491933 | 75494100 | 75494179 | 75491723 | 75491782 | 75508823 | 75508935 | 3.94E−04 | 1.67E−02 | 0.083 |
| 28285 | PVT1 | chr8 | + | 128070159 | 128070361 | 128082752 | 128082848 | 127989161 | 127989291 | 128096517 | 128096654 | 4.55E−04 | 1.86E−02 | 0.082 |
| 41597 | COG8 | chr16 | − | 69330812 | 69331095 | 69332713 | 69332882 | 69326912 | 69329179 | 69334520 | 69335348 | 1.15E−04 | 6.78E−03 | 0.082 |
| 29956 | FKBP15 | chr9 | − | 113206508 | 113206578 | 113207211 | 113207296 | 113202960 | 113203035 | 113211476 | 113211592 | 6.52E−06 | 7.66E−04 | 0.081 |
| 35714 | PLBD1-AS1 | chr12 | + | 14614521 | 14614570 | 14619151 | 14619373 | 14612878 | 14612926 | 14619737 | 14622721 | 1.84E−03 | 4.75E−02 | 0.081 |
| 3426 | TOM1 | chr22 | + | 35345724 | 35345784 | 35346929 | 35346969 | 35338712 | 35338788 | 35347054 | 35347973 | 2.07E−04 | 1.06E−02 | 0.08 |
| 6058 | SUGP1 | chr19 | − | 19296988 | 19297344 | 19310096 | 19310200 | 19280184 | 19280291 | 19316421 | 19316593 | 3.68E−04 | 1.59E−02 | 0.08 |
| 13134 | MCTP1 | chr5 | − | 94708511 | 94708609 | 94710817 | 94710927 | 94703740 | 94705203 | 94714776 | 94714886 | 1.49E−04 | 8.27E−03 | 0.08 |
| 15756 | ZNF185 | chrX | + | 152920706 | 152920748 | 152922172 | 152922256 | 152918982 | 152919081 | 152931671 | 152931776 | 2.82E−04 | 1.31E−02 | 0.08 |
| 23290 | TAF1D | chr11 | − | 93733086 | 93733121 | 93733349 | 93733397 | 93732243 | 93732291 | 93734624 | 93734660 | 1.34E−04 | 7.59E−03 | 0.08 |
| 23311 | CELF1 | chr11 | − | 47476845 | 47476959 | 47477296 | 47477425 | 47475335 | 47475521 | 47478876 | 47478940 | 7.81E−04 | 2.68E−02 | 0.08 |
| 27839 | PIGG | chr4 | + | 515972 | 516185 | 523458 | 523913 | 508828 | 508970 | 527038 | 527230 | 2.92E−05 | 2.40E−03 | 0.08 |
| 12044 | C11orf80 | chr11 | + | 66762078 | 66762450 | 66788159 | 66788269 | 66759042 | 66759096 | 66796280 | 66796364 | 1.34E−03 | 3.85E−02 | 0.079 |
| 16784 | ZNF638 | chr2 | + | 71408121 | 71408247 | 71418601 | 71418639 | 71406127 | 71406262 | 71422813 | 71424038 | 1.37E−04 | 7.73E−03 | 0.079 |
| 29791 | TRIM14 | chr9 | − | 98091908 | 98092001 | 98094866 | 98095029 | 98084352 | 98088005 | 98099930 | 98100164 | 4.02E−04 | 1.70E−02 | 0.079 |
| 3335 | NEIL2 | chr8 | + | 11771445 | 11771585 | 11783202 | 11783399 | 11769709 | 11770335 | 11785962 | 11787345 | 1.37E−03 | 3.91E−02 | 0.078 |
| 17614 | SNX9 | chr6 | + | 157896826 | 157896998 | 157901897 | 157902045 | 157875050 | 157875176 | 157906127 | 157906212 | 1.56E−03 | 4.25E−02 | 0.078 |
| 30960 | NSUN6 | chr10 | − | 18585948 | 18586093 | 18596207 | 18596327 | 18551822 | 18551971 | 18609844 | 18609926 | 1.08E−03 | 3.29E−02 | 0.078 |
| 36710 | MRTFA | chr22 | − | 40420846 | 40421100 | 40423535 | 40423685 | 40420404 | 40420576 | 40424205 | 40424381 | 8.33E−04 | 2.79E−02 | 0.078 |
| 4679 | SLC3A2 | chr11 | + | 62884631 | 62884690 | 62885176 | 62885608 | 62884454 | 62884525 | 62888134 | 62888169 | 2.27E−05 | 1.97E−03 | 0.077 |
| 20835 | SNAPC1 | chr14 | + | 61782246 | 61782397 | 61792806 | 61792902 | 61778847 | 61778910 | 61794948 | 61796428 | 3.88E−04 | 1.65E−02 | 0.077 |
| 33006 | ING4 | chr12 | − | 6652944 | 6653050 | 6656726 | 6656798 | 6652661 | 6652770 | 6663064 | 6663101 | 8.36E−04 | 2.80E−02 | 0.077 |
| 39769 | SERF2 | chr15 | + | 43801131 | 43801187 | 43801517 | 43801569 | 43799357 | 43800784 | 43801710 | 43804427 | 5.05E−04 | 2.00E−02 | 0.077 |
| 6765 | FAM114A2 | chr5 | − | 154034277 | 154034377 | 154034743 | 154034967 | 154033790 | 154033883 | 154038292 | 154038866 | 1.69E−05 | 1.56E−03 | 0.076 |
| 23310 | CELF1 | chr11 | − | 47476845 | 47476956 | 47477296 | 47477425 | 47475335 | 47475521 | 47478876 | 47478940 | 1.20E−03 | 3.58E−02 | 0.076 |
| 32339 | NFKB2 | chr10 | + | 102398211 | 102398297 | 102398738 | 102398864 | 102397980 | 102398085 | 102399287 | 102399497 | 8.96E−04 | 2.91E−02 | 0.076 |
| 16748 | VARS1 | chr6 | − | 31782529 | 31782633 | 31782720 | 31782845 | 31782284 | 31782443 | 31783095 | 31783186 | 7.82E−04 | 2.68E−02 | 0.074 |
| 21441 | CLEC17A | chr19 | + | 14599716 | 14599812 | 14600030 | 14600182 | 14597098 | 14597161 | 14610063 | 14611157 | 1.59E−08 | 6.01E−06 | 0.074 |
| 42394 | TMEM184B | chr22 | − | 38225423 | 38225593 | 38226778 | 38226870 | 38224784 | 38224979 | 38230668 | 38230744 | 1.04E−03 | 3.21E−02 | 0.074 |
| 44865 | EML2 | chr19 | − | 45619059 | 45619191 | 45626704 | 45626839 | 45617629 | 45617697 | 45629950 | 45630046 | 1.51E−04 | 8.38E−03 | 0.074 |
| 4203 | CCM2 | chr7 | + | 45038252 | 45038426 | 45064462 | 45064646 | 45027671 | 45027797 | 45068442 | 45068579 | 2.24E−04 | 1.13E−02 | 0.073 |
| 6358 | WRNIP1 | chr6 | + | 2770119 | 2770361 | 2779262 | 2779492 | 2768690 | 2768882 | 2783405 | 2783561 | 1.09E−03 | 3.31E−02 | 0.073 |
| 13834 | DENND4B | chr1 | − | 153932641 | 153932777 | 153932860 | 153933030 | 153932203 | 153932440 | 153933196 | 153933319 | 4.34E−04 | 1.79E−02 | 0.073 |
| 16516 | AL392172.1 | chr1 | + | 222835118 | 222835215 | 222836995 | 222837066 | 222823855 | 222823945 | 222837218 | 222837383 | 2.53E−06 | 3.62E−04 | 0.073 |
| 22593 | TMEM131 | chr2 | − | 97796843 | 97796986 | 97797364 | 97797516 | 97796217 | 97796404 | 97801894 | 97801961 | 5.40E−04 | 2.09E−02 | 0.073 |
| 27038 | RYK | chr3 | − | 134191848 | 134191974 | 134195081 | 134195182 | 134188836 | 134188923 | 134202729 | 134202874 | 8.45E−04 | 2.82E−02 | 0.073 |
| 38200 | TMCO3 | chr13 | + | 113534044 | 113534240 | 113547299 | 113547379 | 113520615 | 113520733 | 113548302 | 113548452 | 1.54E−05 | 1.45E−03 | 0.073 |
| 8775 | FOXP1 | chr3 | − | 70965889 | 70966056 | 70970735 | 70970805 | 70952816 | 70959391 | 70972554 | 70972676 | 1.92E−05 | 1.71E−03 | 0.072 |
| 44096 | DNMT1 | chr19 | − | 10139675 | 10139817 | 10140780 | 10140909 | 10138438 | 10138605 | 10141104 | 10141189 | 5.58E−05 | 3.96E−03 | 0.072 |
| 44667 | ZNF254 | chr19 | + | 24105939 | 24106066 | 24106547 | 24106643 | 24103746 | 24103867 | 24126253 | 24126320 | 1.88E−04 | 9.86E−03 | 0.072 |
| 1556 | TCFL5 | chr20 | − | 62854015 | 62854157 | 62859363 | 62859526 | 62845137 | 62846117 | 62860124 | 62860308 | 1.47E−03 | 4.11E−02 | 0.071 |
| 19207 | PILRB | chr7 | + | 100355142 | 100355240 | 100356749 | 100356883 | 100353209 | 100353270 | 100358219 | 100358366 | 1.71E−03 | 4.53E−02 | 0.071 |
| 24642 | IDH1 | chr2 | − | 208239070 | 208239233 | 208241993 | 208242145 | 208236226 | 208237169 | 208243426 | 208243604 | 5.73E−05 | 4.03E−03 | 0.071 |
| 32974 | NUP98 | chr11 | − | 3699081 | 3699348 | 3702462 | 3702892 | 3695448 | 3695606 | 3705199 | 3705356 | 1.24E−03 | 3.65E−02 | 0.071 |
| 33668 | DDB2 | chr11 | + | 47234756 | 47234934 | 47235269 | 47235412 | 47234572 | 47234672 | 47237836 | 47238001 | 4.86E−04 | 1.95E−02 | 0.071 |
| 36722 | IGHD | chr14 | − | 105840900 | 105841224 | 105841960 | 105842032 | 105840366 | 105840690 | 105844824 | 105844926 | 3.46E−04 | 1.52E−02 | 0.071 |
| 3587 | POLR2J3 | chr7 | − | 102543524 | 102543702 | 102566581 | 102566736 | 102541500 | 102541662 | 102566996 | 102567083 | 3.00E−05 | 2.44E−03 | 0.07 |
| 15689 | UBAP2L | chr1 | + | 154266500 | 154266568 | 154268756 | 154268954 | 154261591 | 154261697 | 154269361 | 154269412 | 2.10E−04 | 1.07E−02 | 0.07 |
| 21932 | TSEN2 | chr3 | + | 12505153 | 12505231 | 12516610 | 12516661 | 12503261 | 12503784 | 12528887 | 12528924 | 2.20E−10 | 1.85E−07 | 0.07 |
| 24736 | COA1 | chr7 | − | 43650611 | 43650712 | 43656032 | 43656153 | 43648599 | 43648652 | 43657215 | 43657269 | 8.68E−11 | 8.23E−08 | 0.07 |
| 41734 | MPHOSPH6 | chr16 | − | 82149308 | 82149403 | 82151423 | 82151514 | 82148161 | 82148863 | 82164081 | 82164194 | 1.94E−03 | 4.90E−02 | 0.07 |
| 44029 | CERS4 | chr19 | + | 8257878 | 8257985 | 8261687 | 8261844 | 8256948 | 8257077 | 8261929 | 8262418 | 7.46E−04 | 2.59E−02 | 0.07 |
| 3627 | AC069281.2 | chr7 | − | 100577272 | 100577389 | 100577496 | 100577558 | 100577085 | 100577154 | 100577663 | 100577740 | 2.88E−04 | 1.33E−02 | 0.069 |
| 6985 | ATP2B4 | chr1 | + | 203722477 | 203722689 | 203723880 | 203723988 | 203721196 | 203721410 | 203727394 | 203727571 | 6.44E−04 | 2.38E−02 | 0.069 |
| 11889 | EIF4G1 | chr3 | + | 184324877 | 184325114 | 184325268 | 184325373 | 184324200 | 184324347 | 184325479 | 184325639 | 8.39E−04 | 2.80E−02 | 0.069 |
| 26029 | WDFY3 | chr4 | − | 84678918 | 84679242 | 84682373 | 84682470 | 84678167 | 84678279 | 84683942 | 84684125 | 7.33E−04 | 2.57E−02 | 0.069 |
| 29897 | ECPAS | chr9 | − | 111378579 | 111378730 | 111383210 | 111383332 | 111376475 | 111376541 | 111384521 | 111384569 | 8.97E−04 | 2.91E−02 | 0.069 |
| 43246 | SLC25A19 | chr17 | − | 75278151 | 75278335 | 75283422 | 75283593 | 75277352 | 75277483 | 75286303 | 75286459 | 1.54E−03 | 4.24E−02 | 0.069 |
| 2762 | STAU2 | chr8 | − | 73697307 | 73697442 | 73709031 | 73709177 | 73688653 | 73688813 | 73738283 | 73738349 | 2.10E−04 | 1.07E−02 | 0.068 |
| 8952 | RPS6KA3 | chrX | − | 20195064 | 20195145 | 20204021 | 20204103 | 20194188 | 20194268 | 20209287 | 20209404 | 1.82E−03 | 4.72E−02 | 0.068 |
| 27590 | DUS2 | chr16 | + | 68075354 | 68075504 | 68076631 | 68076719 | 68074033 | 68074155 | 68078444 | 68078518 | 2.32E−04 | 1.15E−02 | 0.068 |
| 30685 | MAN1B1 | chr9 | + | 137101483 | 137101672 | 137106124 | 137106315 | 137101004 | 137101153 | 137106688 | 137106809 | 4.34E−05 | 3.26E−03 | 0.068 |
| 36099 | KMT2D | chr12 | − | 49022589 | 49022875 | 49024577 | 49024708 | 49022279 | 49022353 | 49024809 | 49024946 | 3.02E−04 | 1.36E−02 | 0.068 |
| 39357 | RRP8 | chr11 | − | 6600668 | 6600775 | 6600925 | 6601602 | 6599942 | 6600582 | 6601851 | 6602215 | 1.43E−03 | 4.03E−02 | 0.068 |
| 42940 | SPAG9 | chr17 | − | 50977107 | 50977221 | 50979745 | 50979917 | 50974770 | 50974947 | 50982523 | 50982672 | 2.91E−04 | 1.33E−02 | 0.068 |
| 1983 | CPNE1 | chr20 | − | 35627279 | 35627413 | 35630740 | 35630795 | 35626566 | 35626803 | 35630900 | 35631034 | 1.22E−03 | 3.61E−02 | 0.067 |
| 21375 | UBXN11 | chr1 | − | 26297426 | 26297481 | 26297961 | 26298062 | 26294204 | 26294331 | 26306591 | 26306633 | 1.60E−04 | 8.74E−03 | 0.067 |
| 29621 | SLC44A2 | chr19 | + | 10635430 | 10635515 | 10636322 | 10636585 | 10635162 | 10635255 | 10636661 | 10636756 | 1.53E−04 | 8.45E−03 | 0.067 |
| 40949 | KDM5A | chr12 | − | 356431 | 356537 | 362962 | 363097 | 355157 | 355249 | 365933 | 366104 | 1.71E−03 | 4.54E−02 | 0.067 |
| 41888 | TNPO1 | chr5 | + | 72897055 | 72897151 | 7290000 | 72900081 | 72896457 | 72896556 | 72900973 | 72901073 | 5.79E−04 | 2.19E−02 | 0.067 |
| 4447 | HEATR5B | chr2 | − | 36988645 | 36988859 | 36990647 | 36990799 | 36980894 | 36981794 | 37000585 | 37000813 | 1.12E−05 | 1.15E−03 | 0.066 |
| 5902 | RNF170 | chr8 | − | 42865415 | 42865489 | 42870003 | 42870112 | 42861744 | 42861855 | 42887727 | 42887871 | 1.36E−03 | 3.88E−02 | 0.066 |
| 17748 | RHOT1 | chr17 | + | 32176160 | 32176213 | 32183170 | 32183272 | 32175961 | 32176015 | 32192200 | 32192299 | 8.29E−05 | 5.33E−03 | 0.066 |
| 1417 | TMEM50B | chr21 | − | 33439213 | 33439297 | 33448781 | 33448879 | 33432797 | 33432802 | 33449233 | 33449410 | 3.44E−04 | 1.52E−02 | 0.065 |
| 42914 | SCLT1 | chr4 | − | 128888678 | 128888774 | 128891058 | 128891137 | 128883992 | 128884539 | 128936654 | 128936851 | 1.28E−03 | 3.72E−02 | 0.065 |
| 43861 | MAN2B1 | chr19 | − | 12648174 | 12648402 | 12649135 | 12649216 | 12647442 | 12647598 | 12649340 | 12649428 | 6.26E−04 | 2.33E−02 | 0.065 |
| 44242 | PCBP1-AS1 | chr2 | − | 69988741 | 69988838 | 69996685 | 69996888 | 69963281 | 69963500 | 70018014 | 70018131 | 9.80E−05 | 6.02E−03 | 0.065 |
| 3628 | AC069281.2 | chr7 | − | 100577496 | 100577558 | 100577663 | 100577740 | 100577272 | 100577389 | 100577821 | 100577912 | 1.37E−04 | 7.73E−03 | 0.064 |
| 6556 | DTNBP1 | chr6 | − | 15637743 | 15637804 | 15651312 | 15651363 | 15627342 | 15627475 | 15652086 | 15652140 | 1.31E−03 | 3.80E−02 | 0.064 |
| 12051 | PINK1 | chr1 | + | 20644489 | 20644672 | 20645559 | 20645723 | 20638128 | 20639992 | 20648504 | 20649231 | 5.97E−04 | 2.24E−02 | 0.064 |
| 36036 | HDAC7 | chr12 | − | 47795905 | 47796016 | 47796214 | 47796298 | 47795586 | 47795767 | 47797016 | 47797142 | 7.99E−07 | 1.47E−04 | 0.064 |
| 37661 | CMTM1 | chr16 | + | 66571136 | 66571270 | 66577103 | 66577202 | 66569935 | 66570094 | 66578830 | 66579135 | 5.09E−04 | 2.01E−02 | 0.064 |
| 43788 | CDC23 | chr5 | − | 138191861 | 138191937 | 138192268 | 138192388 | 138191473 | 138191535 | 138192503 | 138192657 | 1.74E−03 | 4.59E−02 | 0.064 |
| 44363 | PCBP1-AS1 | chr2 | − | 70055713 | 70055910 | 70083506 | 70083687 | 70051202 | 70051305 | 70085546 | 70085624 | 6.51E−04 | 2.40E−02 | 0.064 |
| 44364 | PCBP1-AS1 | chr2 | − | 70055713 | 70055910 | 70083537 | 70083687 | 70051202 | 70051305 | 70085546 | 70085624 | 7.09E−04 | 2.51E−02 | 0.064 |
| 18703 | EHBP1L1 | chr11 | + | 65579935 | 65579989 | 65580080 | 65580259 | 65579340 | 65579436 | 65580336 | 65580479 | 9.66E−04 | 3.05E−02 | 0.063 |
| 24801 | ANKZF1 | chr2 | + | 219234132 | 219234288 | 219234825 | 219235312 | 219233714 | 219233943 | 219235473 | 219235585 | 1.21E−03 | 3.60E−02 | 0.063 |
| 28378 | AC118553.2 | chr1 | + | 100049908 | 100050004 | 100058665 | 100058728 | 100043072 | 100043229 | 100059877 | 100060005 | 1.04E−06 | 1.80E−04 | 0.063 |
| 32358 | NAE1 | chr16 | − | 66816580 | 66816672 | 66816964 | 66817028 | 66813786 | 66813846 | 66817424 | 66817487 | 3.00E−05 | 2.44E−03 | 0.063 |
| 35148 | GIT1 | chr17 | − | 29575629 | 29575703 | 29575811 | 29575898 | 29575287 | 29575470 | 29576077 | 29576131 | 3.75E−04 | 1.61E−02 | 0.063 |
| 40779 | CREBBP | chr16 | − | 3770569 | 3770986 | 3773750 | 3773930 | 3769173 | 3769353 | 3774568 | 3774693 | 1.03E−03 | 3.19E−02 | 0.063 |
| 2063 | RALY | chr20 | + | 34073818 | 34073866 | 34075873 | 34076040 | 34073562 | 34073635 | 34078504 | 34078553 | 1.24E−05 | 1.24E−03 | 0.062 |
| 2718 | XAF1 | chr17 | + | 6759255 | 6759718 | 6760405 | 6760601 | 6758088 | 6758224 | 6762154 | 6762240 | 1.26E−08 | 4.95E−06 | 0.062 |
| 4191 | CCM2 | chr7 | + | 45038252 | 45038426 | 45063917 | 45064001 | 45000187 | 45000363 | 45073459 | 45073571 | 5.75E−04 | 2.18E−02 | 0.062 |
| 5619 | CPSF7 | chr11 | − | 61416104 | 61416162 | 61416363 | 61416519 | 61415665 | 61415784 | 61419948 | 61420094 | 6.88E−05 | 4.58E−03 | 0.062 |
| 7468 | CAST | chr5 | + | 96740037 | 96740118 | 96740744 | 96740783 | 96737848 | 96737947 | 96741265 | 96741358 | 1.66E−03 | 4.42E−02 | 0.062 |
| 19129 | SMG5 | chr1 | − | 156259004 | 156259163 | 156260450 | 156260626 | 156253448 | 156253508 | 156261332 | 156261408 | 4.67E−05 | 3.45E−03 | 0.062 |
| 28274 | PVT1 | chr8 | + | 128070159 | 128070272 | 128082752 | 128082848 | 127989161 | 127989291 | 128096517 | 128096654 | 7.86E−05 | 5.12E−03 | 0.062 |
| 38793 | RGS6 | chr14 | + | 72518350 | 72518537 | 72536185 | 72536275 | 72510153 | 72510279 | 72562416 | 72562467 | 4.43E−04 | 1.82E−02 | 0.062 |
| 44930 | SUMF2 | chr7 | + | 56074173 | 56074218 | 56074585 | 56074736 | 56072996 | 56073111 | 56076833 | 56076889 | 9.56E−04 | 3.03E−02 | 0.062 |
| 2061 | RALY | chr20 | + | 34073818 | 34073866 | 34075873 | 34076040 | 34073562 | 34073635 | 34077027 | 34077245 | 1.26E−05 | 1.25E−03 | 0.061 |
| 2065 | RALY | chr20 | + | 34073818 | 34073866 | 34075873 | 34076040 | 34073562 | 34073635 | 34079909 | 34084884 | 1.29E−05 | 1.27E−03 | 0.061 |
| 6581 | MLKL | chr16 | − | 74678898 | 74678980 | 74682650 | 74682786 | 74675612 | 74675764 | 74685485 | 74685583 | 4.87E−04 | 1.96E−02 | 0.061 |
| 11114 | ITPA | chr20 | + | 3218516 | 3218632 | 3221840 | 3221917 | 3215211 | 3215312 | 3223365 | 3223449 | 6.31E−05 | 4.30E−03 | 0.061 |
| 13915 | GATAD2A | chr19 | + | 19494293 | 19494383 | 19495753 | 19495885 | 19492580 | 19492712 | 19496051 | 19496219 | 7.80E−04 | 2.68E−02 | 0.061 |
| 16309 | MKNK1 | chr1 | − | 46568442 | 46568498 | 46572062 | 46572167 | 46565040 | 46565136 | 46574946 | 46575020 | 1.04E−03 | 3.21E−02 | 0.061 |
| 23799 | MIR4435-2HG | chr2 | − | 111248147 | 111248241 | 111344060 | 111344237 | 111239801 | 111239996 | 111367486 | 111367575 | 1.18E−03 | 3.53E−02 | 0.061 |
| 42499 | SSBP4 | chr19 | + | 18427897 | 18427982 | 18430840 | 18430930 | 18427751 | 18427813 | 18431352 | 18431418 | 2.11E−05 | 1.85E−03 | 0.061 |
| 4188 | CCM2 | chr7 | + | 45038252 | 45038426 | 45063917 | 45064001 | 45000187 | 45000363 | 45072725 | 45072783 | 9.67E−04 | 3.05E−02 | 0.06 |
| 26429 | TNFRSF1A | chr12 | − | 6329777 | 6330066 | 6330852 | 6330926 | 6328756 | 6329622 | 6333068 | 6333147 | 6.58E−04 | 2.41E−02 | 0.06 |
| 40102 | TLE3 | chr15 | − | 70056297 | 70056374 | 70057458 | 70057658 | 70055048 | 70055298 | 70058158 | 70058291 | 1.09E−04 | 6.55E−03 | 0.06 |
| 20024 | ARHGAP25 | chr2 | + | 68775220 | 68775420 | 68782232 | 68782320 | 68734810 | 68735260 | 68787839 | 68787956 | 8.98E−04 | 2.91E−02 | 0.059 |
| 27329 | TRAK1 | chr3 | + | 42200817 | 42201054 | 42202435 | 42202752 | 42199176 | 42199253 | 42209766 | 42209985 | 1.82E−03 | 4.71E−02 | 0.059 |
| 31707 | LRCH3 | chr3 | + | 197858833 | 197858905 | 197865422 | 197865471 | 197854391 | 197854445 | 197866111 | 197866219 | 1.69E−03 | 4.50E−02 | 0.059 |
| 40103 | TLE3 | chr15 | − | 70056297 | 70056374 | 70057473 | 70057658 | 70055048 | 70055298 | 70058158 | 70058291 | 1.24E−04 | 7.18E−03 | 0.059 |
| 44810 | PLD3 | chr19 | + | 40366772 | 40366915 | 40367695 | 40367879 | 40366609 | 40366684 | 40369907 | 40370011 | 1.83E−03 | 4.72E−02 | 0.059 |
| 1844 | ADA | chr20 | − | 44623006 | 44623078 | 44624201 | 44624329 | 44622828 | 44622930 | 44625568 | 44625684 | 4.33E−04 | 1.79E−02 | 0.058 |
| 18484 | SHISA5 | chr3 | − | 48469360 | 48469573 | 48469727 | 48469843 | 48467875 | 48469186 | 48473009 | 48473208 | 2.43E−04 | 1.19E−02 | 0.058 |
| 20510 | NCF1 | chr7 | + | 74787983 | 74788088 | 74788558 | 74788704 | 74785181 | 74785299 | 74789038 | 74789376 | 2.97E−04 | 1.35E−02 | 0.058 |
| 33100 | NUP88 | chr17 | − | 5387778 | 5387904 | 5388801 | 5388960 | 5387385 | 5387670 | 5391560 | 5391662 | 5.16E−04 | 2.03E−02 | 0.058 |
| 41469 | USB1 | chr16 | + | 58014272 | 58014326 | 58017333 | 58017439 | 58009928 | 58010112 | 58018971 | 58019055 | 3.30E−04 | 1.46E−02 | 0.058 |
| 42777 | FMNL1 | chr17 | + | 45240475 | 45240625 | 45241128 | 45241230 | 45238954 | 45239065 | 45241381 | 45241634 | 6.87E−04 | 2.48E−02 | 0.058 |
| 8604 | CYB5R4 | chr6 | + | 83914415 | 83914448 | 83922437 | 83922470 | 83909008 | 83909090 | 83924469 | 83924592 | 7.92E−05 | 5.14E−03 | 0.057 |
| 32341 | NFKB2 | chr10 | + | 102398384 | 102398523 | 102399287 | 102399497 | 102398211 | 102398297 | 102399576 | 102399718 | 1.05E−03 | 3.23E−02 | 0.057 |
| 33101 | NUP88 | chr17 | − | 5387778 | 5387952 | 5388801 | 5388960 | 5387604 | 5387670 | 5391560 | 5391662 | 1.07E−03 | 3.26E−02 | 0.057 |
| 36501 | RCBTB2 | chr13 | − | 48511769 | 48511877 | 48512015 | 48512174 | 48510628 | 48510771 | 48512728 | 48512895 | 1.44E−03 | 4.06E−02 | 0.057 |
| 37703 | STRADA | chr17 | − | 63713405 | 63713527 | 63714005 | 63714108 | 63710727 | 63710836 | 63726637 | 63726695 | 4.23E−04 | 1.76E−02 | 0.057 |
| 40002 | CRTC2 | chr1 | − | 153951259 | 153951666 | 153952017 | 153952262 | 153949114 | 153949384 | 153952396 | 153952446 | 8.94E−04 | 2.91E−02 | 0.057 |
| 43264 | UNC13D | chr17 | − | 75839838 | 75839942 | 75840017 | 75840110 | 75836800 | 75836918 | 75840224 | 75840304 | 2.71E−04 | 1.28E−02 | 0.057 |
| 8369 | ICE1 | chr5 | + | 5460435 | 5465226 | 5466333 | 5466502 | 5457331 | 5457741 | 5468827 | 5468988 | 1.78E−03 | 4.65E−02 | 0.056 |
| 13557 | TMBIM1 | chr2 | − | 218278514 | 218278565 | 218279037 | 218279091 | 218277934 | 218277974 | 218279288 | 218279353 | 1.05E−05 | 1.12E−03 | 0.056 |
| 25013 | SH2D3C | chr9 | − | 127747146 | 127747271 | 127749210 | 127749665 | 127744563 | 127745099 | 127751171 | 127751300 | 2.10E−04 | 1.07E−02 | 0.056 |
| 29620 | SLC44A2 | chr19 | + | 10635162 | 10635255 | 10635430 | 10635515 | 10634755 | 10635073 | 10636322 | 10636585 | 4.12E−04 | 1.73E−02 | 0.056 |
| 35066 | SORL1 | chr11 | + | 121488031 | 121488193 | 121490042 | 121490110 | 121478117 | 121478243 | 121496868 | 121497049 | 8.62E−05 | 5.49E−03 | 0.056 |
| 35395 | LTBR | chr12 | + | 6386346 | 6386444 | 6388397 | 6388505 | 6386065 | 6386162 | 6388799 | 6388825 | 1.54E−03 | 4.23E−02 | 0.056 |
| 39701 | CHP1 | chr15 | + | 41262755 | 41262883 | 41270556 | 41270618 | 41256909 | 41256990 | 41278766 | 41278889 | 6.70E−05 | 4.48E−03 | 0.056 |
| 42041 | CTDNEP1 | chr17 | − | 7244550 | 7244635 | 7246025 | 7246137 | 7243590 | 7244245 | 7246253 | 7246370 | 5.60E−04 | 2.14E−02 | 0.056 |
| 20271 | RPS12 | chr6 | + | 132816460 | 132816563 | 132816959 | 132817061 | 132814971 | 132815224 | 132817479 | 132817564 | 1.94E−05 | 1.72E−03 | 0.055 |
| 31375 | WDFY4 | chr10 | + | 48963841 | 48964054 | 48966525 | 48966673 | 48959721 | 48959813 | 48969063 | 48969248 | 1.73E−03 | 4.58E−02 | 0.055 |
| 31757 | FANCA | chr16 | − | 89748658 | 89748767 | 89749729 | 89749902 | 89746830 | 89746890 | 89758576 | 89758705 | 4.75E−05 | 3.50E−03 | 0.055 |
| 1984 | CPNE1 | chr20 | − | 35627279 | 35627413 | 35630438 | 35631034 | 35626566 | 35626803 | 35631113 | 35631173 | 8.80E−04 | 2.89E−02 | 0.054 |
| 1985 | CPNE1 | chr20 | − | 35627279 | 35627413 | 35630900 | 35631034 | 35626566 | 35626803 | 35631113 | 35631173 | 7.38E−04 | 2.58E−02 | 0.054 |
| 11215 | OSBPL11 | chr3 | − | 125563697 | 125563843 | 125567393 | 125567595 | 125560378 | 125560519 | 125576188 | 125576365 | 7.18E−04 | 2.53E−02 | 0.054 |
| 17747 | RHOT1 | chr17 | + | 32176160 | 32176213 | 32182756 | 32182865 | 32175961 | 32176015 | 32183170 | 32183272 | 6.47E−04 | 2.39E−02 | 0.054 |
| 41189 | ITGAL | chr16 | + | 30513770 | 30513846 | 30516972 | 30517086 | 30511049 | 30511136 | 30517648 | 30517705 | 8.72E−04 | 2.88E−02 | 0.054 |
| 23035 | INTS8 | chr8 | + | 94831991 | 94832174 | 94836523 | 94836631 | 94828974 | 94829026 | 94838462 | 94838618 | 1.42E−03 | 4.02E−02 | 0.053 |
| 24937 | DBNL | chr7 | + | 44058431 | 44058480 | 44058901 | 44058983 | 44057781 | 44058280 | 44059353 | 44059449 | 6.45E−04 | 2.39E−02 | 0.053 |
| 30060 | GSN | chr9 | + | 121321267 | 121321401 | 121324553 | 121324644 | 121318664 | 121318880 | 121326511 | 121326682 | 5.82E−04 | 2.20E−02 | 0.053 |
| 7203 | CNOT3 | chr19 | + | 54146600 | 54146657 | 54148147 | 54148535 | 54145909 | 54146043 | 54148619 | 54148699 | 2.93E−04 | 1.34E−02 | 0.052 |
| 12005 | DAGLB | chr7 | − | 6421726 | 6421804 | 6425987 | 6426114 | 6416840 | 6416921 | 6432836 | 6432959 | 1.40E−04 | 7.83E−03 | 0.052 |
| 24938 | DBNL | chr7 | + | 44058431 | 44058480 | 44058877 | 44058983 | 44058128 | 44058280 | 44059353 | 44059449 | 6.28E−04 | 2.34E−02 | 0.052 |
| 29289 | STXBP2 | chr19 | + | 7645196 | 7645306 | 7646248 | 7646344 | 7644613 | 7644752 | 7647161 | 7647247 | 3.42E−04 | 1.51E−02 | 0.052 |
| 34644 | SNHG29 | chr17 | + | 16439527 | 16439703 | 16440184 | 16440253 | 16439326 | 16439414 | 16441367 | 16442028 | 1.38E−06 | 2.20E−04 | 0.052 |
| 43261 | UNC13D | chr17 | − | 75831097 | 75831169 | 75831242 | 75831348 | 75830577 | 75830661 | 75832965 | 75833045 | 7.85E−04 | 2.69E−02 | 0.052 |
| 44479 | FCHO1 | chr19 | + | 17766668 | 17766810 | 17770424 | 17770577 | 17764374 | 17764449 | 17770791 | 17770827 | 6.78E−04 | 2.46E−02 | 0.052 |
| 1774 | SLC35C2 | chr20 | − | 46350760 | 46350890 | 46352050 | 46352226 | 46345979 | 46350524 | 46354881 | 46354965 | 1.51E−03 | 4.19E−02 | 0.051 |
| 14640 | WDR47 | chr1 | − | 108981732 | 108981864 | 108982608 | 108982785 | 108974535 | 108974754 | 108983281 | 108983451 | 2.15E−07 | 4.90E−05 | 0.051 |
| 19480 | MKNK2 | chr19 | − | 2041039 | 2041204 | 2041839 | 2042034 | 2038247 | 2040177 | 2042426 | 2042522 | 1.08E−03 | 3.30E−02 | 0.051 |
| 25060 | INPP5D | chr2 | + | 233125747 | 233125919 | 233130507 | 233130648 | 233122106 | 233122257 | 233139841 | 233139929 | 1.78E−03 | 4.65E−02 | 0.051 |
| 31271 | LRCH4 | chr7 | − | 100576901 | 100577005 | 100577496 | 100577558 | 100576693 | 100576777 | 100577663 | 100577740 | 9.38E−04 | 3.01E−02 | 0.051 |
| 33014 | TTC7A | chr2 | + | 47011330 | 47011435 | 47021861 | 47021979 | 47006640 | 47006724 | 47023407 | 47023465 | 3.55E−04 | 1.55E−02 | 0.051 |
| 34325 | UNC93B1 | chr11 | − | 67997674 | 67997799 | 67998358 | 67998452 | 67996601 | 67996784 | 67999172 | 67999305 | 1.33E−03 | 3.84E−02 | 0.051 |
| 40254 | PDE4B | chr1 | + | 66363406 | 66363571 | 66365666 | 66365766 | 66363167 | 66363266 | 66367695 | 66367850 | 1.84E−03 | 4.76E−02 | 0.051 |
| 8593 | HLA-DRB1 | chr6 | − | 32581556 | 32581838 | 32584108 | 32584378 | 32580745 | 32580856 | 32589642 | 32589848 | 3.00E−07 | 6.47E−05 | 0.05 |
| 26427 | TNFRSF1A | chr12 | − | 6329777 | 6330066 | 6330266 | 6330295 | 6328756 | 6329622 | 6330597 | 6330711 | 7.09E−04 | 2.51E−02 | 0.05 |
| 28374 | AC118553.2 | chr1 | + | 100017681 | 100017815 | 100049908 | 100050004 | 100015301 | 100015420 | 100058665 | 100058728 | 8.74E−04 | 2.88E−02 | 0.05 |
| 32012 | FGR | chr1 | − | 27615688 | 27615844 | 27616856 | 27617006 | 27615433 | 27615613 | 27617192 | 27617296 | 5.53E−04 | 2.13E−02 | 0.05 |
| 41634 | COG4 | chr16 | − | 70481358 | 70481487 | 70481763 | 70481865 | 70480567 | 70481144 | 70482091 | 70482175 | 1.98E−03 | 4.97E−02 | 0.05 |
| 5822 | FLOT1 | chr6 | − | 30740678 | 30740798 | 30741613 | 30741704 | 30740495 | 30740591 | 30741791 | 30741867 | 1.07E−03 | 3.27E−02 | −0.05 |
| 7218 | CAMK1D | chr10 | + | 12791157 | 12791233 | 12814194 | 12814307 | 12769672 | 12769799 | 12816249 | 12816328 | 3.83E−04 | 1.64E−02 | −0.05 |
| 26168 | LMBR1 | chr7 | − | 156724111 | 156724178 | 156734176 | 156734257 | 156680876 | 156684163 | 156756392 | 156756465 | 5.37E−04 | 2.09E−02 | −0.05 |
| 30140 | TSC2 | chr16 | + | 2058746 | 2058873 | 2060669 | 2060813 | 2057104 | 2057178 | 2060952 | 2061039 | 6.70E−11 | 6.49E−08 | −0.05 |
| 32024 | FGR | chr1 | − | 27621558 | 27621657 | 27625088 | 27625151 | 27617192 | 27617296 | 27635064 | 27635185 | 1.94E−05 | 1.72E−03 | −0.05 |
| 33292 | NAA60 | chr16 | + | 3448470 | 3448540 | 3479470 | 3479600 | 3443705 | 3443827 | 3482501 | 3482598 | 4.67E−11 | 4.73E−08 | −0.05 |
| 41720 | INTS6 | chr13 | − | 51395299 | 51395483 | 51423029 | 51423090 | 51387385 | 51387540 | 51430293 | 51430383 | 5.78E−07 | 1.11E−04 | −0.05 |
| 6653 | TTC16 | chr9 | + | 127724755 | 127724897 | 127726238 | 127726404 | 127724119 | 127724364 | 127726969 | 127727112 | 2.22E−04 | 1.12E−02 | −0.051 |
| 7255 | H2AZ2 | chr7 | − | 44835528 | 44835658 | 44843276 | 44843354 | 44831977 | 44834562 | 44847968 | 44848087 | 3.68E−04 | 1.59E−02 | −0.051 |
| 17900 | AC004997.1 | chr22 | − | 30288705 | 30288776 | 30292772 | 30292851 | 30287372 | 30287560 | 30293650 | 30293805 | 1.39E−08 | 5.32E−06 | −0.051 |
| 29606 | SPTLC1 | chr9 | − | 92076797 | 92077035 | 92079433 | 92079566 | 92067965 | 92068098 | 92080015 | 92080088 | 7.21E−04 | 2.54E−02 | −0.051 |
| 32122 | BAG6 | chr6 | − | 31640384 | 31640528 | 31640644 | 31640704 | 31640198 | 31640306 | 31640791 | 31640938 | 2.42E−04 | 1.19E−02 | −0.051 |
| 33514 | TTC17 | chr11 | + | 43414589 | 43414776 | 43436163 | 43436334 | 43407352 | 43407577 | 43443324 | 43443584 | 7.31E−04 | 2.56E−02 | −0.051 |
| 36731 | CDK16 | chrX | + | 47227378 | 47227469 | 47228566 | 47228644 | 47227159 | 47227223 | 47228730 | 47229251 | 1.51E−03 | 4.19E−02 | −0.051 |
| 38149 | NAXD | chr13 | + | 110622215 | 110622366 | 110624233 | 110624279 | 110615590 | 110615647 | 110627438 | 110627547 | 1.48E−05 | 1.41E−03 | −0.051 |
| 39325 | IGFLR1 | chr19 | − | 35739709 | 35740086 | 35740379 | 35740564 | 35739256 | 35739626 | 35741023 | 35741223 | 6.10E−04 | 2.28E−02 | −0.051 |
| 3618 | GOLGA7 | chr8 | + | 41490832 | 41490965 | 41497508 | 41497661 | 41490481 | 41490617 | 41505910 | 41506012 | 1.17E−06 | 1.96E−04 | −0.052 |
| 10432 | YY1AP1 | chr1 | − | 155679408 | 155679512 | 155680415 | 155680456 | 155670319 | 155670464 | 155688070 | 155688201 | 4.95E−08 | 1.49E−05 | −0.052 |
| 10433 | YY1AP1 | chr1 | − | 155679408 | 155679512 | 155680415 | 155680456 | 155675009 | 155675096 | 155688070 | 155688201 | 2.74E−08 | 9.63E−06 | −0.052 |
| 11519 | CASC4 | chr15 | + | 44328684 | 44328787 | 44338236 | 44338317 | 44322964 | 44323019 | 44379689 | 44379788 | 1.88E−03 | 4.83E−02 | −0.052 |
| 23465 | PTDSS1 | chr8 | + | 96284108 | 96284153 | 96287021 | 96287146 | 96273298 | 96273390 | 96295097 | 96295256 | 6.11E−04 | 2.28E−02 | −0.052 |
| 28076 | CEP290 | chr12 | − | 88059897 | 88060020 | 88060829 | 88060994 | 88055575 | 88055717 | 88062691 | 88062778 | 4.15E−04 | 1.73E−02 | −0.052 |
| 31401 | TIMM23B-AGAP6 | chr10 | + | 49973011 | 49973142 | 49987164 | 49987234 | 49958369 | 49958480 | 49988750 | 49988938 | 3.22E−05 | 2.58E−03 | −0.052 |
| 41036 | IL21R | chr16 | + | 27430055 | 27430120 | 27434346 | 27434449 | 27403079 | 27403220 | 27437487 | 27437687 | 1.39E−04 | 7.82E−03 | −0.052 |
| 44107 | DNMT1 | chr19 | − | 10180777 | 10180885 | 10182040 | 10182077 | 10180349 | 10180569 | 10194819 | 10194953 | 1.01E−04 | 6.20E−03 | −0.052 |
| 3280 | PDIA4 | chr7 | − | 149005140 | 149005374 | 149005896 | 149006053 | 149003061 | 149004209 | 149008158 | 149008310 | 1.26E−04 | 7.24E−03 | −0.053 |
| 3969 | SIPA1 | chr11 | + | 65647383 | 65647658 | 65649261 | 65649480 | 65646455 | 65647065 | 65649560 | 65649672 | 1.26E−03 | 3.69E−02 | −0.053 |
| 4385 | POLR2F | chr22 | + | 37956772 | 37956842 | 37959345 | 37959476 | 37953696 | 37953807 | 37967098 | 37967170 | 1.81E−03 | 4.70E−02 | −0.053 |
| 5438 | POLL | chr10 | − | 101582762 | 101582891 | 101583507 | 101583681 | 101580247 | 101580416 | 101584877 | 101584919 | 1.75E−03 | 4.60E−02 | −0.053 |
| 19703 | PSMA5 | chr1 | − | 109415236 | 109415363 | 109421859 | 109421926 | 109411952 | 109413135 | 109426301 | 109426427 | 1.91E−04 | 1.00E−02 | −0.053 |
| 20366 | PAFAH2 | chr1 | − | 25989447 | 25989601 | 25990726 | 25990921 | 25988230 | 25988327 | 25998024 | 25998044 | 4.13E−04 | 1.73E−02 | −0.053 |
| 24383 | MOV10 | chr1 | + | 112674847 | 112675049 | 112688934 | 112689138 | 112674486 | 112674729 | 112689414 | 112689650 | 3.24E−04 | 1.45E−02 | −0.053 |
| 24451 | HMGB2 | chr4 | − | 173333068 | 173333214 | 173333499 | 173333669 | 173332820 | 173332995 | 173334271 | 173334358 | 1.37E−03 | 3.91E−02 | −0.053 |
| 24732 | PYCR2 | chr1 | − | 225922203 | 225922383 | 225923700 | 225923771 | 225921857 | 225922079 | 225924043 | 225924250 | 1.31E−03 | 3.79E−02 | −0.053 |
| 35445 | B4GALT3 | chr1 | − | 161173858 | 161174049 | 161176433 | 161176579 | 161173604 | 161173727 | 161177422 | 161177479 | 9.86E−04 | 3.10E−02 | −0.053 |
| 40663 | RHOT2 | chr16 | + | 672488 | 672566 | 672702 | 672825 | 672253 | 672384 | 672927 | 673130 | 1.56E−03 | 4.25E−02 | −0.053 |
| 757 | DDX17 | chr22 | − | 38494629 | 38494802 | 38495795 | 38495937 | 38494020 | 38494131 | 38498084 | 38498150 | 1.60E−03 | 4.33E−02 | −0.054 |
| 9267 | NQO2 | chr6 | + | 3004494 | 3004651 | 3006467 | 3006559 | 3003668 | 3003789 | 3010024 | 3010189 | 0.00E+00 | 0.00E+00 | −0.054 |
| 11532 | WDR70 | chr5 | + | 37437921 | 37437981 | 37443238 | 37443372 | 37396374 | 37396570 | 37479833 | 37479987 | 8.58E−04 | 2.84E−02 | −0.054 |
| 23044 | INTS8 | chr8 | + | 94841490 | 94841591 | 94842346 | 94842488 | 94838462 | 94838618 | 94849461 | 94849532 | 1.96E−03 | 4.93E−02 | −0.054 |
| 25751 | EXOSC1 | chr10 | − | 97441170 | 97441259 | 97445731 | 97445847 | 97438662 | 97438703 | 97445954 | 97445973 | 5.51E−04 | 2.13E−02 | −0.054 |
| 33783 | RABEPK | chr9 | + | 125203007 | 125203066 | 125213369 | 125213522 | 125200779 | 125200906 | 125220326 | 125220397 | 7.05E−08 | 1.97E−05 | −0.054 |
| 42447 | UTP6 | chr17 | − | 31884423 | 31884505 | 31885979 | 31886061 | 31880180 | 31880754 | 31887235 | 31887313 | 4.06E−04 | 1.71E−02 | −0.054 |
| 2013 | TRPC4AP | chr20 | − | 35021189 | 35021356 | 35035122 | 35035308 | 35016007 | 35016139 | 35044504 | 35044712 | 2.04E−04 | 1.05E−02 | −0.055 |
| 2723 | XAF1 | chr17 | + | 6759661 | 6759718 | 6762154 | 6762240 | 6758088 | 6758224 | 6770642 | 6770984 | 1.45E−03 | 4.06E−02 | −0.055 |
| 4411 | ZDHHC4 | chr7 | + | 6578565 | 6578720 | 6580554 | 6580678 | 6577433 | 6577720 | 6581606 | 6581680 | 6.44E−04 | 2.38E−02 | −0.055 |
| 6440 | PHYKPL | chr5 | − | 178224447 | 17822456 | 178231404 | 178231523 | 178222851 | 178222934 | 178232491 | 178232802 | 1.14E−03 | 3.45E−02 | −0.055 |
| 7454 | CAST | chr5 | + | 96675538 | 96675601 | 96695835 | 96695907 | 96662202 | 96662497 | 96722638 | 96722698 | 1.34E−03 | 3.85E−02 | −0.055 |
| 9739 | PPA2 | chr4 | − | 105437949 | 105438036 | 105453597 | 105453642 | 105424195 | 105424322 | 105456680 | 105456745 | 1.53E−03 | 4.22E−02 | −0.055 |
| 26076 | RBM6 | chr3 | + | 49972058 | 49972148 | 49975322 | 49975392 | 49962575 | 49962685 | 50048244 | 50048319 | 6.68E−04 | 2.44E−02 | −0.055 |
| 31571 | DDX21 | chr10 | + | 68974669 | 68974743 | 68977528 | 68977688 | 68973544 | 68973664 | 68978841 | 68978976 | 6.66E−04 | 2.43E−02 | −0.055 |
| 36498 | RCBTB2 | chr13 | − | 48502723 | 48502914 | 48510628 | 48510771 | 48501741 | 48501868 | 48511769 | 48511877 | 1.52E−03 | 4.19E−02 | −0.055 |
| 42367 | ABCA7 | chr19 | + | 1050920 | 1051052 | 1051154 | 1051294 | 1049265 | 1049437 | 1051448 | 1051586 | 2.25E−04 | 1.13E−02 | −0.055 |
| 566 | LMF2 | chr22 | − | 50506284 | 50506502 | 50506637 | 50506666 | 50506034 | 50506213 | 50506781 | 50507035 | 8.30E−06 | 9.30E−04 | −0.056 |
| 20059 | PHC2 | chr1 | − | 33332274 | 33332404 | 33334184 | 33334292 | 33331347 | 33331462 | 33349596 | 33349772 | 1.12E−03 | 3.38E−02 | −0.056 |
| 23916 | WDSUB1 | chr2 | − | 159248371 | 159248512 | 159259809 | 159259843 | 159235792 | 159236190 | 159271701 | 159271795 | 4.05E−06 | 5.21E−04 | −0.056 |
| 24950 | JAK1 | chr1 | − | 64883276 | 64883475 | 64886258 | 64886341 | 64879024 | 64879148 | 64966332 | 64966361 | 8.22E−04 | 2.77E−02 | −0.056 |
| 29542 | IFTAP | chr11 | + | 36610080 | 36610239 | 36633283 | 36633438 | 36594492 | 36594761 | 36636050 | 36636117 | 1.35E−03 | 3.86E−02 | −0.056 |
| 33784 | RABEPK | chr9 | + | 125207563 | 125207721 | 125213369 | 125213522 | 125203007 | 125203066 | 125220326 | 125220397 | 3.56E−10 | 2.72E−07 | −0.056 |
| 4249 | SNX10 | chr7 | + | 26346419 | 26346466 | 26360974 | 26361061 | 26291861 | 26292086 | 26364534 | 26364635 | 4.98E−04 | 1.98E−02 | −0.057 |
| 12019 | DAGLB | chr7 | − | 6432836 | 6432959 | 6445952 | 6446104 | 6425987 | 6426114 | 6447747 | 6447932 | 1.23E−04 | 7.12E−03 | −0.057 |
| 12968 | DEK | chr6 | − | 18257952 | 18258062 | 18258303 | 18258405 | 18256360 | 18256455 | 18263842 | 18263996 | 1.88E−03 | 4.82E−02 | −0.057 |
| 23917 | WDSUB1 | chr2 | − | 159248371 | 159248512 | 159271701 | 159271795 | 159235792 | 159236190 | 159275545 | 159275638 | 1.02E−04 | 6.24E−03 | −0.057 |
| 31125 | LAIR1 | chr19 | − | 54360915 | 54361209 | 54364294 | 54364330 | 54360021 | 54360072 | 54364770 | 54364807 | 3.23E−04 | 1.45E−02 | −0.057 |
| 31834 | VDAC2 | chr10 | + | 75212229 | 75212298 | 75214020 | 75214070 | 75211133 | 75211189 | 75217887 | 75217991 | 5.50E−06 | 6.71E−04 | −0.057 |
| 31943 | PPP4R3B | chr2 | − | 55598415 | 55599039 | 55603977 | 55604076 | 55588878 | 55588956 | 55615450 | 55615506 | 1.84E−03 | 4.75E−02 | −0.057 |
| 37940 | DLEU2 | chr13 | − | 50044639 | 50044768 | 50049583 | 50049662 | 50029277 | 50029492 | 50068096 | 50068163 | 1.43E−04 | 7.98E−03 | −0.057 |
| 39170 | STK38 | chr6 | − | 36525590 | 36525642 | 36540071 | 36540207 | 36524340 | 36524463 | 36547189 | 36547479 | 6.60E−05 | 4.44E−03 | −0.057 |
| 42639 | STAT5B | chr17 | − | 42227528 | 42227685 | 42231999 | 42232137 | 42224778 | 42224868 | 42276247 | 42276391 | 1.58E−03 | 4.28E−02 | −0.057 |
| 700 | DESI1 | chr22 | − | 41604043 | 41604153 | 41607261 | 41607331 | 41603258 | 41603381 | 41607839 | 41607861 | 4.08E−04 | 1.71E−02 | −0.058 |
| 4195 | CCM2 | chr7 | + | 45038252 | 45038426 | 45069825 | 45069961 | 45000248 | 45000363 | 45073459 | 45073571 | 1.11E−05 | 1.15E−03 | −0.058 |
| 4501 | PREX1 | chr20 | − | 48708259 | 48708421 | 48747808 | 48747880 | 48700752 | 48700886 | 48827641 | 48827999 | 2.87E−04 | 1.33E−02 | −0.058 |
| 8547 | TMEM87A | chr15 | − | 42233212 | 42233306 | 42236319 | 42236419 | 42231191 | 42231260 | 42237431 | 42237615 | 1.27E−03 | 3.71E−02 | −0.058 |
| 10943 | NEPRO | chr3 | − | 113011044 | 113011406 | 113012733 | 113013403 | 113010642 | 113010704 | 113013952 | 113014032 | 5.40E−04 | 2.09E−02 | −0.058 |
| 28639 | WDR37 | chr10 | + | 1072115 | 1072293 | 1077906 | 1078003 | 1056403 | 1056564 | 1080010 | 1080106 | 3.99E−07 | 8.20E−05 | −0.058 |
| 31124 | LAIR1 | chr19 | − | 54360915 | 54361206 | 54364294 | 54364330 | 54360021 | 54360072 | 54364770 | 54364995 | 2.96E−04 | 1.35E−02 | −0.058 |
| 31126 | LAIR1 | chr19 | − | 54360915 | 54361243 | 54364294 | 54364330 | 54360021 | 54360072 | 54364770 | 54364931 | 3.16E−04 | 1.42E−02 | −0.058 |
| 36293 | SUPT20H | chr13 | − | 37047534 | 37047601 | 37047877 | 37047936 | 37045246 | 37045373 | 37051487 | 37051583 | 1.59E−03 | 4.30E−02 | −0.058 |
| 38204 | CDC16 | chr13 | + | 114236644 | 114236699 | 114236798 | 114236896 | 114234886 | 114235132 | 114238989 | 114239028 | 1.05E−03 | 3.22E−02 | −0.058 |
| 40415 | JPX | chrX | + | 73946998 | 73947374 | 73994863 | 73994961 | 73944583 | 73944662 | 73998767 | 73998844 | 1.20E−03 | 3.57E−02 | −0.058 |
| 44053 | RBM23 | chr14 | − | 22911785 | 22911903 | 22913723 | 22913845 | 22911327 | 22911403 | 22918998 | 22919149 | 3.25E−06 | 4.40E−04 | −0.058 |
| 44493 | RPL18A | chr19 | + | 17861292 | 17861472 | 17862093 | 17862223 | 17859909 | 17859974 | 17862917 | 17863027 | 6.70E−04 | 2.44E−02 | −0.058 |
| 85 | LINC00893 | chrX | − | 149529512 | 149529593 | 149532881 | 149533055 | 149529366 | 149529428 | 149533629 | 149534374 | 5.96E−10 | 4.26E−07 | −0.059 |
| 4414 | ZDHHC4 | chr7 | + | 6578565 | 6578746 | 6580554 | 6580678 | 6577467 | 6577720 | 6581606 | 6581680 | 1.19E−04 | 6.97E−03 | −0.059 |
| 4597 | SND1 | chr7 | + | 127686612 | 127686762 | 127694827 | 127694948 | 127652193 | 127652451 | 127698874 | 127698953 | 1.79E−03 | 4.67E−02 | −0.059 |
| 4674 | SLC3A2 | chr11 | + | 62881892 | 62882066 | 62882907 | 62882999 | 62880871 | 62881447 | 62884456 | 62884525 | 1.10E−04 | 6.58E−03 | −0.059 |
| 5544 | RBM33 | chr7 | + | 155700772 | 155700944 | 155706859 | 155707068 | 155680668 | 155680908 | 155711172 | 155711455 | 4.11E−05 | 3.14E−03 | −0.059 |
| 9328 | TMEM63A | chr1 | − | 225867887 | 225868030 | 225871075 | 225871113 | 225867111 | 225867163 | 225871986 | 225872053 | 9.97E−04 | 3.12E−02 | −0.059 |
| 13598 | MEF2D | chr1 | − | 156477011 | 156477202 | 156479289 | 156479346 | 156475107 | 156475237 | 156479585 | 156479796 | 1.75E−03 | 4.59E−02 | −0.059 |
| 35081 | NLRC5 | chr16 | + | 57028071 | 57028385 | 57029772 | 57029856 | 57026638 | 57027018 | 57029994 | 57030084 | 1.43E−03 | 4.03E−02 | −0.059 |
| 45057 | POLD2 | chr7 | − | 44117942 | 44118064 | 44121833 | 44122109 | 44117583 | 44117742 | 44123510 | 44123532 | 1.22E−03 | 3.62E−02 | −0.059 |
| 4790 | TNFSF13 | chr17 | + | 7559623 | 7559702 | 7560048 | 7560167 | 7558475 | 7559297 | 7560349 | 7560488 | 6.48E−04 | 2.39E−02 | −0.06 |
| 9916 | NTAN1 | chr16 | − | 15044333 | 15044407 | 15047417 | 15047550 | 15041622 | 15041676 | 15047854 | 15047920 | 1.89E−03 | 4.83E−02 | −0.06 |
| 22426 | VPS53 | chr17 | − | 661808 | 661895 | 697417 | 697484 | 655837 | 655953 | 710532 | 710613 | 2.79E−04 | 1.30E−02 | −0.06 |
| 23901 | TANGO2 | chr22 | + | 20036759 | 20037094 | 20043354 | 20043443 | 20021138 | 20021246 | 20052464 | 20052584 | 1.92E−06 | 2.87E−04 | −0.06 |
| 27969 | ARHGEF40 | chr14 | + | 21073044 | 21073242 | 21073931 | 21075180 | 21070272 | 21070399 | 21075331 | 21075553 | 7.07E−04 | 2.51E−02 | −0.06 |
| 34244 | ZDHHC24 | chr11 | − | 66526935 | 66527006 | 66543703 | 66543981 | 66524154 | 66524276 | 66545722 | 66546036 | 1.85E−03 | 4.76E−02 | −0.06 |
| 42374 | ABCA7 | chr19 | + | 1055906 | 1055939 | 1056065 | 1056243 | 1055082 | 1055351 | 1056329 | 1056499 | 9.44E−04 | 3.02E−02 | −0.06 |
| 3713 | SAMD9L | chr7 | − | 93144731 | 93144833 | 93145384 | 93145532 | 93130762 | 93135991 | 93145908 | 93146040 | 7.31E−06 | 8.43E−04 | −0.061 |
| 6535 | EPSTI1 | chr13 | − | 42953947 | 42954021 | 42969093 | 42970670 | 42926335 | 42926429 | 42991977 | 42992271 | 3.94E−04 | 1.67E−02 | −0.061 |
| 10849 | POLR3C | chr1 | + | 145833489 | 145833582 | 145838055 | 145838206 | 145833259 | 145833364 | 145839889 | 145839991 | 2.24E−04 | 1.13E−02 | −0.061 |
| 17923 | ECHDC1 | chr6 | − | 127316449 | 127316502 | 127330808 | 127331030 | 127314815 | 127314896 | 127343335 | 127343609 | 1.90E−03 | 4.84E−02 | −0.061 |
| 20812 | GMDS-DT | chr6 | + | 2269697 | 2269799 | 2271815 | 2273417 | 2263600 | 2263684 | 2360543 | 2362108 | 5.82E−08 | 1.74E−05 | −0.061 |
| 23923 | WDSUB1 | chr2 | − | 159256195 | 159256375 | 159271701 | 159271795 | 159248371 | 159248512 | 159275545 | 159275638 | 9.35E−05 | 5.83E−03 | −0.061 |
| 24591 | TYROBP | chr19 | − | 35907445 | 35907580 | 35907729 | 35907762 | 35907217 | 35907261 | 35908167 | 35908295 | 2.51E−04 | 1.21E−02 | −0.061 |
| 25076 | TTC13 | chr1 | − | 230931297 | 230931472 | 230931735 | 230931877 | 230928936 | 230929093 | 230933778 | 230933861 | 1.03E−03 | 3.19E−02 | −0.061 |
| 28367 | PHF20L1 | chr8 | + | 132814636 | 132814889 | 132816887 | 132817076 | 132811045 | 132811128 | 132817338 | 132817545 | 1.24E−03 | 3.65E−02 | −0.061 |
| 28514 | CORO1C | chr12 | − | 108662028 | 108662158 | 108701123 | 108701323 | 108658737 | 108658919 | 108731428 | 108731596 | 1.44E−03 | 4.06E−02 | −0.061 |
| 17921 | ECHDC1 | chr6 | − | 127316449 | 127316502 | 127326997 | 127327144 | 127314815 | 127314896 | 127330808 | 127331030 | 7.33E−05 | 4.80E−03 | −0.062 |
| 5102 | SMIM8 | chr6 | + | 87337008 | 87337166 | 87371592 | 87371712 | 87322604 | 87322632 | 87375461 | 87375710 | 1.06E−07 | 2.80E−05 | −0.063 |
| 10941 | NEPRO | chr3 | − | 113011044 | 113011165 | 113012733 | 113013403 | 113010642 | 113010704 | 113013952 | 113014032 | 3.12E−04 | 1.41E−02 | −0.063 |
| 14464 | HLA-C | chr6 | − | 31269337 | 31269385 | 31269492 | 31269525 | 31268748 | 31269173 | 31269965 | 31270085 | 3.84E−04 | 1.64E−02 | −0.063 |
| 18942 | BBS9 | chr7 | + | 33357854 | 33357995 | 33367766 | 33367862 | 33349067 | 33349170 | 33383665 | 33383838 | 2.87E−05 | 2.37E−03 | −0.063 |
| 35912 | AMD1 | chr6 | + | 110887504 | 110887591 | 110888856 | 110888983 | 110874784 | 110875215 | 110890253 | 110890356 | 1.32E−03 | 3.82E−02 | −0.063 |
| 44977 | ERCC1 | chr19 | − | 45413962 | 45414034 | 45414860 | 45414960 | 45413676 | 45413745 | 45416820 | 45416897 | 8.87E−05 | 5.63E−03 | −0.063 |
| 12722 | DLG1 | chr3 | − | 197194424 | 197194589 | 197282678 | 197282845 | 197142717 | 197142768 | 197296345 | 197296400 | 2.35E−04 | 1.16E−02 | −0.064 |
| 31413 | ZFAND1 | chr8 | − | 81713917 | 81714039 | 81714803 | 81715114 | 81702968 | 81703124 | 81718181 | 81718224 | 1.54E−06 | 2.41E−04 | −0.064 |
| 37049 | MAPKAPK5 | chr12 | + | 111865249 | 111865323 | 111867571 | 111867669 | 111842227 | 111842769 | 111868752 | 111868861 | 1.52E−03 | 4.19E−02 | −0.064 |
| 23304 | CELF1 | chr11 | − | 47473087 | 47473231 | 47475335 | 47475521 | 47472190 | 47472357 | 47476845 | 47476956 | 1.14E−03 | 3.45E−02 | −0.065 |
| 28227 | PVT1 | chr8 | + | 127795893 | 127796008 | 128070159 | 128070272 | 127794682 | 127794734 | 128096517 | 128096654 | 1.97E−03 | 4.96E−02 | −0.065 |
| 29161 | WASH3P | chr15 | + | 101966069 | 101966223 | 101971822 | 101972049 | 101961617 | 101961641 | 101972407 | 101972465 | 1.18E−05 | 1.20E−03 | −0.065 |
| 35667 | CNOT2 | chr12 | + | 70278131 | 70278274 | 70310894 | 70311017 | 70242993 | 70243480 | 70319297 | 70319364 | 1.91E−06 | 2.87E−04 | −0.065 |
| 42479 | RFFL | chr17 | − | 35021370 | 35021781 | 35026373 | 35026561 | 35016369 | 35016580 | 35089104 | 35089295 | 5.51E−09 | 2.40E−06 | −0.065 |
| 13333 | LONP2 | chr16 | + | 48258617 | 48258740 | 48261423 | 48261587 | 48252130 | 48252365 | 48262777 | 48262872 | 2.97E−05 | 2.43E−03 | −0.066 |
| 25217 | ARIH2 | chr3 | + | 48961611 | 48961679 | 48967124 | 48967275 | 48927461 | 48927813 | 48968533 | 48968655 | 3.59E−04 | 1.56E−02 | −0.066 |
| 12217 | SNX22 | chr15 | + | 64152637 | 64152742 | 64153651 | 64153684 | 64151714 | 64151850 | 64153934 | 64154903 | 1.85E−04 | 9.74E−03 | −0.067 |
| 12379 | SCAP | chr3 | − | 47417121 | 47417207 | 47417303 | 47417823 | 47415097 | 47415180 | 47418133 | 47418249 | 9.43E−04 | 3.02E−02 | −0.067 |
| 16211 | VASP | chr19 | + | 45522717 | 45522818 | 45523840 | 45523877 | 45522339 | 45522581 | 45524096 | 45524142 | 1.24E−03 | 3.66E−02 | −0.067 |
| 29953 | FKBP15 | chr9 | − | 113199813 | 113199963 | 113202530 | 113202629 | 113198854 | 113198923 | 113202960 | 113203035 | 7.85E−04 | 2.69E−02 | −0.067 |
| 22981 | PSD4 | chr2 | + | 113184956 | 113185073 | 113185364 | 113185440 | 113182345 | 113183512 | 113185876 | 113186255 | 1.62E−03 | 4.36E−02 | −0.068 |
| 23041 | INTS8 | chr8 | + | 94838462 | 94838618 | 94842346 | 94842488 | 94836523 | 94836631 | 94849461 | 94849532 | 3.63E−04 | 1.57E−02 | −0.068 |
| 23898 | TANGO2 | chr22 | + | 20036759 | 20036854 | 20043354 | 20043443 | 20021068 | 20021246 | 20052464 | 20052584 | 4.69E−06 | 5.87E−04 | −0.068 |
| 26239 | TMEM127 | chr2 | − | 96254832 | 96254997 | 96265137 | 96265512 | 96248513 | 96254115 | 96265868 | 96265997 | 6.96E−04 | 2.50E−02 | −0.068 |
| 31705 | LRCH3 | chr3 | + | 197854391 | 197854445 | 197858833 | 197858905 | 197852560 | 197852620 | 197865422 | 197865471 | 2.28E−04 | 1.14E−02 | −0.068 |
| 34542 | KDM5C | chrX | − | 53218275 | 53218398 | 53220838 | 53220916 | 53217795 | 53217966 | 53224739 | 53225422 | 7.86E−04 | 2.69E−02 | −0.068 |
| 38056 | BBS1 | chr11 | + | 66519616 | 66519748 | 66521269 | 66521376 | 66515860 | 66515933 | 66523171 | 66523365 | 4.89E−06 | 6.09E−04 | −0.068 |
| 6520 | SRP14-AS1 | chr15 | + | 40056452 | 40056641 | 40064295 | 40064601 | 40045960 | 40046069 | 40065220 | 40065705 | 1.78E−03 | 4.65E−02 | −0.069 |
| 11629 | SOS1 | chr2 | − | 38987472 | 38987591 | 38995122 | 38995387 | 38981548 | 38986315 | 38996921 | 38997038 | 1.28E−03 | 3.72E−02 | −0.069 |
| 12817 | RNF121 | chr11 | + | 71994718 | 71994852 | 71995439 | 71995551 | 71990596 | 71990717 | 71996194 | 71997597 | 9.06E−04 | 2.93E−02 | −0.069 |
| 20927 | PDCD6 | chr5 | + | 272710 | 272772 | 311292 | 311402 | 271620 | 271821 | 314416 | 314974 | 8.73E−04 | 2.88E−02 | −0.069 |
| 30589 | SEC16A | chr9 | − | 136451255 | 136451408 | 136453427 | 136453510 | 136448083 | 136448161 | 136454108 | 136454327 | 5.17E−06 | 6.37E−04 | −0.069 |
| 42425 | ADAP2 | chr17 | + | 30931888 | 30931968 | 30934184 | 30934297 | 30922939 | 30923070 | 30944906 | 30945053 | 3.24E−10 | 2.54E−07 | −0.069 |
| 3555 | DNAJC2 | chr7 | − | 103337735 | 103337811 | 103341763 | 103341865 | 103327655 | 103327754 | 103344558 | 103344622 | 1.81E−03 | 4.70E−02 | −0.07 |
| 10981 | RPS6KB1 | chr17 | + | 59914634 | 59914703 | 59926434 | 59926582 | 59912683 | 59912804 | 59930116 | 59930174 | 1.20E−04 | 6.99E−03 | −0.07 |
| 12816 | RNF121 | chr11 | + | 71994718 | 71994852 | 71995449 | 71995551 | 71990596 | 71990717 | 71996194 | 71996275 | 6.57E−04 | 2.41E−02 | −0.07 |
| 28077 | CEP290 | chr12 | − | 88059897 | 88060020 | 88060829 | 88060994 | 88058847 | 88059020 | 88062691 | 88062778 | 1.87E−03 | 4.82E−02 | −0.07 |
| 28229 | PVT1 | chr8 | + | 127795893 | 127796071 | 128070159 | 128070272 | 127794564 | 127794734 | 128096517 | 128096654 | 2.73E−04 | 1.28E−02 | −0.07 |
| 44010 | EXOSC9 | chr4 | + | 121802914 | 121803017 | 121804621 | 121804759 | 121802673 | 121802793 | 121807539 | 121807622 | 7.79E−04 | 2.68E−02 | −0.07 |
| 44969 | CLPTM1 | chr19 | + | 44961962 | 44962075 | 44973086 | 44973210 | 44955379 | 44955467 | 44974438 | 44974597 | 2.53E−04 | 1.22E−02 | −0.07 |
| 4504 | PREX1 | chr20 | − | 48734545 | 48734650 | 48747808 | 48747880 | 48726289 | 48726391 | 48827641 | 48827999 | 8.83E−04 | 2.89E−02 | −0.071 |
| 14881 | IL15 | chr4 | + | 141688821 | 141689035 | 141720468 | 141720566 | 141656185 | 141656307 | 141721058 | 141721177 | 2.01E−06 | 2.98E−04 | −0.071 |
| 39256 | MTA1 | chr14 | + | 105445417 | 105445511 | 105450057 | 105450184 | 105438671 | 105438739 | 105450260 | 105450324 | 3.53E−04 | 1.54E−02 | −0.071 |
| 42809 | EFCAB13 | chr17 | + | 47361377 | 47361521 | 47391436 | 47391580 | 47347807 | 47347951 | 47394024 | 47394099 | 1.54E−04 | 8.50E−03 | −0.071 |
| 44459 | OCEL1 | chr19 | + | 17226993 | 17227199 | 17228255 | 17228309 | 17226692 | 17226869 | 17229008 | 17229219 | 1.47E−07 | 3.56E−05 | −0.071 |
| 6564 | ZC3H7A | chr16 | − | 11763477 | 11763659 | 11765052 | 11765153 | 11762670 | 11762747 | 11765488 | 11765561 | 3.00E−05 | 2.44E−03 | −0.072 |
| 12000 | DAGLB | chr7 | − | 6416258 | 6416754 | 6416840 | 6416921 | 6412986 | 6413034 | 6421726 | 6421804 | 1.11E−16 | 8.07E−13 | −0.072 |
| 20740 | EIF2B4 | chr2 | − | 27368371 | 27368463 | 27369005 | 27369212 | 27368024 | 27368113 | 27369413 | 27369549 | 7.43E−04 | 2.59E−02 | −0.072 |
| 22606 | EIF2D | chr1 | − | 206599002 | 206599092 | 206599462 | 206599612 | 206597099 | 206597195 | 206599732 | 206599836 | 7.11E−04 | 2.51E−02 | −0.072 |
| 1113 | GUSBP11 | chr22 | − | 23705428 | 23705541 | 23709129 | 23709241 | 23694118 | 23695517 | 23709785 | 23709867 | 1.21E−05 | 1.23E−03 | −0.073 |
| 16016 | FBXO3 | chr11 | − | 33768850 | 33769014 | 33770740 | 33770830 | 33758486 | 33758601 | 33774393 | 33774504 | 6.96E−04 | 2.50E−02 | −0.073 |
| 16210 | VASP | chr19 | + | 45522717 | 45522818 | 45523643 | 45523695 | 45522339 | 45522581 | 45523840 | 45523877 | 6.90E−04 | 2.49E−02 | −0.073 |
| 28047 | GRK3 | chr22 | + | 25703509 | 25703576 | 25704108 | 25704209 | 25695106 | 25695214 | 25709897 | 25709964 | 8.06E−04 | 2.73E−02 | −0.073 |
| 32173 | PIK3AP1 | chr10 | − | 96656797 | 96656934 | 96709566 | 96709983 | 96652697 | 96652842 | 96720381 | 96720514 | 8.41E−04 | 2.81E−02 | −0.073 |
| 38205 | CDC16 | chr13 | + | 114236644 | 114236699 | 114236801 | 114236896 | 114234978 | 114235132 | 114238989 | 114239028 | 2.81E−04 | 1.31E−02 | −0.073 |
| 3362 | RALGAPB | chr20 | + | 38565358 | 38565478 | 38570768 | 38570847 | 38562531 | 38562697 | 38574149 | 38574298 | 1.66E−03 | 4.42E−02 | −0.074 |
| 9220 | PAPSS1 | chr4 | − | 107693770 | 107694006 | 107701170 | 107701285 | 107687038 | 107687177 | 107720119 | 107720234 | 1.11E−04 | 6.61E−03 | −0.074 |
| 12770 | RABGAP1L | chr1 | + | 174370978 | 174371072 | 174393994 | 174394145 | 174304985 | 174305127 | 174637374 | 174637488 | 2.10E−04 | 1.07E−02 | −0.074 |
| 14479 | C1orf21 | chr1 | + | 184477385 | 184477603 | 184507587 | 184507682 | 184387028 | 184387368 | 184590738 | 184590815 | 4.36E−04 | 1.80E−02 | −0.074 |
| 31122 | LAIR1 | chr19 | − | 54360021 | 54360072 | 54361009 | 54361209 | 54356927 | 54356966 | 54364294 | 54364330 | 8.91E−04 | 2.91E−02 | −0.074 |
| 31689 | ST6GAL1 | chr3 | + | 187038741 | 187038873 | 187042653 | 187043310 | 186963784 | 186963926 | 187051248 | 187051346 | 2.92E−04 | 1.34E−02 | −0.074 |
| 7301 | OGFOD1 | chr16 | + | 56466151 | 56466268 | 56466875 | 56466967 | 56462533 | 56462634 | 56467164 | 56467198 | 1.90E−03 | 4.84E−02 | −0.075 |
| 26360 | ARAP2 | chr4 | − | 36046709 | 36046849 | 36052005 | 36052053 | 36045978 | 36046076 | 36057969 | 36058143 | 8.93E−10 | 5.64E−07 | −0.075 |
| 27608 | FDX1 | chr11 | + | 110435833 | 110435958 | 110456917 | 110457047 | 110429947 | 110430305 | 110462353 | 110464884 | 1.93E−03 | 4.88E−02 | −0.075 |
| 28749 | TTC39B | chr9 | − | 15225916 | 15226012 | 15267913 | 15267948 | 15214138 | 15214249 | 15307083 | 15307360 | 1.34E−03 | 3.86E−02 | −0.075 |
| 44030 | CD320 | chr19 | − | 8302776 | 8302980 | 8303854 | 8304088 | 8302126 | 8302605 | 8305030 | 8305156 | 1.32E−03 | 3.81E−02 | −0.075 |
| 4923 | RUNX3 | chr1 | − | 24919239 | 24919344 | 24927573 | 24927730 | 24907258 | 24907417 | 24929586 | 24929852 | 1.76E−03 | 4.61E−02 | −0.076 |
| 19452 | COP1 | chr1 | − | 176163814 | 176163891 | 176175909 | 176176007 | 176162868 | 176162988 | 176184632 | 176184692 | 1.21E−03 | 3.59E−02 | −0.076 |
| 25935 | ANKRD27 | chr19 | − | 32644324 | 32644479 | 32646458 | 32646615 | 32643571 | 32643631 | 32649681 | 32649792 | 6.73E−04 | 2.45E−02 | −0.076 |
| 36155 | TXLNA | chr1 | + | 32180313 | 32180514 | 32184524 | 32184616 | 32179674 | 32179776 | 32187953 | 32188124 | 2.11E−04 | 1.08E−02 | −0.076 |
| 43598 | TPGS2 | chr18 | − | 36807846 | 36807934 | 36818893 | 36818973 | 36805373 | 36805502 | 36828682 | 36828730 | 2.80E−04 | 1.31E−02 | −0.076 |
| 13149 | MCTP1 | chr5 | − | 94942347 | 94942427 | 94953218 | 94953361 | 94940083 | 94940195 | 95017366 | 95017484 | 1.74E−03 | 4.58E−02 | −0.077 |
| 21116 | AD000671.1 | chr19 | + | 35752803 | 35752833 | 35752913 | 35752999 | 35752433 | 35752484 | 35753086 | 35753253 | 3.45E−04 | 1.52E−02 | −0.077 |
| 24714 | COQ7 | chr16 | + | 19075720 | 19075860 | 19077305 | 19077374 | 19071927 | 19072106 | 19078080 | 19078283 | 3.53E−05 | 2.77E−03 | −0.077 |
| 27874 | NRF1 | chr7 | + | 129657342 | 129657574 | 129671428 | 129671543 | 129611755 | 129611824 | 129677631 | 129677758 | 3.72E−04 | 1.60E−02 | −0.077 |
| 29548 | IFTAP | chr11 | + | 36610080 | 36610239 | 36633283 | 36633438 | 36594771 | 36594915 | 36636050 | 36636117 | 1.55E−05 | 1.45E−03 | −0.077 |
| 38413 | SCFD1 | chr14 | + | 30633946 | 30634037 | 30638124 | 30638247 | 30630476 | 30630565 | 30639776 | 30639864 | 1.62E−03 | 4.36E−02 | −0.077 |
| 7044 | LY96 | chr8 | + | 74010000 | 74010129 | 74026788 | 74026841 | 73991425 | 73991554 | 74028955 | 74029074 | 1.18E−03 | 3.53E−02 | −0.078 |
| 19303 | TMCO4 | chr1 | − | 19770541 | 19770569 | 19780579 | 19780766 | 19755633 | 19755766 | 19787025 | 19787117 | 1.51E−03 | 4.18E−02 | −0.078 |
| 24799 | ANKZF1 | chr2 | + | 219231927 | 219232040 | 219232259 | 219232362 | 219230227 | 219230405 | 219232489 | 219232683 | 2.03E−04 | 1.05E−02 | −0.078 |
| 34178 | SCYL1 | chr11 | + | 65537966 | 65538182 | 65538269 | 65538324 | 65537811 | 65537880 | 65538441 | 65538704 | 1.02E−03 | 3.17E−02 | −0.078 |
| 2313 | PTPRA | chr20 | + | 2923206 | 2923285 | 2986764 | 2986849 | 2873488 | 2873760 | 2988031 | 2988105 | .99E−04 | 2.50E−02 | −0.079 |
| 15394 | DRAM1 | chr12 | + | 101897862 | 101897930 | 101908185 | 101908363 | 101877579 | 101877920 | 101914173 | 101914232 | 4.25E−04 | 1.77E−02 | −0.079 |
| 24882 | USP48 | chr1 | − | 21747066 | 21747149 | 21748137 | 21748271 | 21736445 | 21736625 | 21751506 | 21751615 | 3.92E−06 | 5.08E−04 | −0.079 |
| 27972 | ARHGEF40 | chr14 | + | 21076562 | 21076643 | 21076773 | 21076890 | 21076359 | 21076456 | 21078176 | 21078207 | 4.71E−04 | 1.91E−02 | −0.079 |
| 37532 | CHFR | chr12 | − | 132877554 | 132877654 | 132887195 | 132887340 | 132871904 | 132872394 | 132887546 | 132887564 | 4.18E−05 | 3.18E−03 | −0.079 |
| 45064 | GYS1 | chr19 | − | 48978097 | 48978157 | 48981529 | 48981636 | 48977923 | 48978002 | 48982254 | 48982375 | 5.02E−04 | 1.99E−02 | −0.079 |
| 6651 | RORC | chr1 | − | 151813238 | 151813346 | 151813487 | 151813702 | 151812946 | 151813057 | 151814573 | 151814695 | 4.60E−08 | 1.41E−05 | −0.08 |
| 17128 | UVRAG | chr11 | + | 76004004 | 76004089 | 76007533 | 76007621 | 75983386 | 75983513 | 76008806 | 76008867 | 1.17E−03 | 3.51E−02 | −0.08 |
| 19217 | SIRPB1 | chr20 | − | 1566152 | 1566267 | 1578337 | 1578694 | 1561384 | 1565497 | 1619868 | 1620009 | 4.88E−07 | 9.67E−05 | −0.08 |
| 37776 | TTI2 | chr8 | − | 33509745 | 33509932 | 33511966 | 33512712 | 33507228 | 33507321 | 33513081 | 33513135 | 1.34E−04 | 7.56E−03 | −0.08 |
| 19445 | COP1 | chr1 | − | 176149005 | 176149074 | 176184632 | 176184692 | 176135009 | 176135086 | 176206571 | 176206978 | 1.35E−03 | 3.86E−02 | −0.081 |
| 22814 | TMEM8B | chr9 | + | 35829876 | 35829955 | 35834460 | 35834650 | 35829227 | 35829391 | 35835010 | 35835218 | 2.02E−07 | 4.64E−05 | −0.081 |
| 28090 | CAPN12 | chr19 | − | 38734318 | 38734389 | 38735369 | 38735429 | 38734141 | 38734204 | 38735501 | 38735544 | 1.83E−04 | 9.71E−03 | −0.081 |
| 31378 | HIPK3 | chr11 | + | 33286412 | 33287511 | 33328509 | 33328633 | 33257380 | 33257889 | 33337074 | 33337194 | 1.89E−03 | 4.83E−02 | −0.081 |
| 1933 | SRC | chr20 | + | 37401601 | 37401678 | 37402434 | 37402588 | 37400114 | 37400294 | 37402748 | 37402880 | 7.44E−04 | 2.59E−02 | −0.082 |
| 4115 | HERPUD2 | chr7 | − | 35667433 | 35667588 | 35694183 | 35694627 | 35638349 | 35638472 | 35694800 | 35695135 | 3.50E−06 | 4.65E−04 | −0.082 |
| 14883 | KLRG1 | chr12 | + | 8992205 | 8992310 | 9008974 | 9009075 | 8989545 | 8989717 | 9009425 | 9010751 | 3.04E−04 | 1.37E−02 | −0.082 |
| 19447 | COP1 | chr1 | − | 176149005 | 176149074 | 176175909 | 176176007 | 176136487 | 176136547 | 176184632 | 176184692 | 2.23E−04 | 1.12E−02 | −0.082 |
| 23929 | WDSUB1 | chr2 | − | 159257757 | 159257864 | 159271701 | 159271795 | 159256195 | 159256375 | 159275545 | 159275638 | 1.53E−03 | 4.22E−02 | −0.082 |
| 41848 | SPG7 | chr16 | + | 89526328 | 89526468 | 89529476 | 89529579 | 89524005 | 89524247 | 89530682 | 89530808 | 2.70E−04 | 1.28E−02 | −0.082 |
| 7970 | P4HA1 | chr10 | − | 73043886 | 73043957 | 73044980 | 73045051 | 73016845 | 73016899 | 73046924 | 73047101 | 1.85E−03 | 4.76E−02 | −0.083 |
| 10088 | IKBKG | chrX | + | 154558531 | 154558650 | 154560406 | 154560559 | 154551987 | 154552189 | 154561687 | 154561784 | 1.24E−09 | 7.49E−07 | −0.083 |
| 12410 | METTL26 | chr16 | − | 634888 | 634956 | 635280 | 635340 | 634718 | 634797 | 635611 | 635774 | 1.93E−04 | 1.01E−02 | −0.083 |
| 19092 | DNAJC1 | chr10 | − | 21920797 | 21920963 | 21929039 | 21929141 | 21919831 | 21919929 | 22003212 | 22003730 | 1.67E−03 | 4.45E−02 | −0.083 |
| 30571 | NACC2 | chr9 | − | 136016264 | 136016429 | 136049635 | 136050580 | 136013863 | 136013969 | 136095188 | 136095289 | 1.49E−03 | 4.14E−02 | −0.083 |
| 34475 | THOC6 | chr16 | + | 3025923 | 3025988 | 3026062 | 3026166 | 3025707 | 3025823 | 3026250 | 3026288 | 2.77E−04 | 1.30E−02 | −0.083 |
| 14053 | ALG13 | chrX | + | 111682131 | 111682235 | 111682235 | 111682294 | 111681169 | 111681424 | 111684964 | 111685103 | 5.04E−05 | 3.67E−03 | −0.084 |
| 16680 | RB1 | chr13 | + | 48376917 | 48377034 | 48380052 | 48380084 | 48373404 | 48373492 | 48380164 | 48380241 | 1.85E−04 | 9.76E−03 | −0.084 |
| 26616 | LRP8 | chr1 | − | 53266472 | 53266647 | 53275630 | 53275753 | 53264168 | 53264396 | 53280586 | 53280715 | 1.81E−03 | 4.70E−02 | −0.084 |
| 43044 | BCAS3 | chr17 | + | 60947218 | 60947352 | 60989970 | 60990235 | 60924406 | 60924500 | 61015750 | 61015901 | 1.06E−03 | 3.26E−02 | −0.084 |
| 5904 | RNF170 | chr8 | − | 42870003 | 42870112 | 42873930 | 42874006 | 42861744 | 42861855 | 42887727 | 42887871 | 8.99E−04 | 2.92E−02 | −0.085 |
| 11094 | MDM4 | chr1 | + | 204530683 | 204530817 | 204532190 | 204532246 | 204526359 | 204526434 | 204537429 | 204537497 | 1.99E−03 | 4.99E−02 | −0.085 |
| 12411 | METTL26 | chr16 | − | 634888 | 635063 | 635280 | 635340 | 634718 | 634797 | 635611 | 635774 | 9.09E−05 | 5.70E−03 | −0.085 |
| 31765 | FANCA | chr16 | − | 89758576 | 89758705 | 89761948 | 89762022 | 89749729 | 89749902 | 89764889 | 89765004 | 1.53E−05 | 1.44E−03 | −0.085 |
| 32246 | CWF19L1 | chr10 | − | 100260217 | 100260319 | 100261978 | 100262063 | 100253420 | 100253539 | 100267570 | 100267680 | 2.05E−04 | 1.06E−02 | −0.085 |
| 35953 | LRRK2 | chr12 | + | 40251464 | 40251544 | 40252909 | 40253016 | 40251231 | 40251374 | 40257247 | 40257377 | 8.92E−05 | 5.63E−03 | −0.085 |
| 8860 | PRMT9 | chr4 | − | 147642786 | 147642940 | 147653851 | 147654566 | 147638959 | 147639082 | 147657791 | 147657975 | 9.03E−04 | 2.92E−02 | −0.086 |
| 9481 | NUP54 | chr4 | − | 76134174 | 76134362 | 76136185 | 76136412 | 76132522 | 76132719 | 76144148 | 76144292 | 3.53E−04 | 1.54E−02 | −0.086 |
| 24796 | ANKZF1 | chr2 | + | 219230227 | 219230405 | 219232259 | 219232362 | 219229805 | 219229900 | 219232489 | 219232683 | 1.41E−03 | 4.00E−02 | −0.086 |
| 32359 | NAE1 | chr16 | − | 66821449 | 66821559 | 66823226 | 66823306 | 66818527 | 66818637 | 66823528 | 66823600 | 1.68E−05 | 1.55E−03 | −0.086 |
| 14506 | PPP2R2D | chr10 | + | 131901237 | 131901330 | 131934457 | 131934555 | 131901007 | 131901155 | 131940030 | 131940196 | 1.25E−03 | 3.66E−02 | −0.087 |
| 21936 | TSEN2 | chr3 | + | 12516610 | 12516661 | 12519058 | 12519197 | 12503261 | 12503784 | 12528887 | 12528924 | 1.51E−12 | 2.19E−09 | −0.087 |
| 10089 | IKBKG | chrX | + | 154558531 | 154558650 | 154560406 | 154560559 | 154556164 | 154556376 | 154561687 | 154561784 | 1.48E−03 | 4.12E−02 | −0.088 |
| 12351 | THEM4 | chr1 | − | 151889213 | 151889373 | 151895007 | 151895194 | 151888272 | 151888383 | 151909359 | 151909502 | 9.09E−04 | 2.93E−02 | −0.088 |
| 19444 | COP1 | chr1 | − | 176149005 | 176149074 | 176175909 | 176176007 | 176135009 | 176135086 | 176184632 | 176184664 | 1.65E−04 | 8.95E−03 | −0.088 |
| 7469 | CAST | chr5 | + | 96740744 | 96740783 | 96741265 | 96741358 | 96740037 | 96740118 | 96741493 | 96741580 | 9.80E−04 | 3.08E−02 | −0.089 |
| 12683 | AGPAT3 | chr21 | + | 43903956 | 43904019 | 43959633 | 43959859 | 43865222 | 43865345 | 43967945 | 43968115 | 2.89E−04 | 1.33E−02 | −0.089 |
| 29062 | TRPM7 | chr15 | − | 50586391 | 50586488 | 50589591 | 50589656 | 50583088 | 50583159 | 50591910 | 50592626 | 2.46E−04 | 1.20E−02 | −0.089 |
| 11570 | RAD1 | chr5 | − | 34911553 | 34911812 | 34914694 | 34914961 | 34909257 | 84909356 | 34915415 | 34915504 | 6.95E−04 | 2.50E−02 | −0.09 |
| 17609 | RNPC3 | chr1 | + | 103527694 | 103527742 | 103533738 | 103533857 | 103525690 | 103526262 | 103534773 | 103534857 | 1.31E−03 | 3.80E−02 | −0.09 |
| 23928 | WDSUB1 | chr2 | − | 159257757 | 159257864 | 159259809 | 159259843 | 159256195 | 159256375 | 159271701 | 159271795 | 2.72E−05 | 2.27E−03 | −0.09 |
| 27854 | SLBP | chr4 | − | 1700010 | 1700070 | 1703595 | 1703700 | 1699563 | 1699701 | 1711873 | 1711990 | 2.46E−04 | 1.20E−02 | −0.09 |
| 42257 | KIF1C | chr17 | + | 4999849 | 4999970 | 5000219 | 5000352 | 4997949 | 4998156 | 5000771 | 5000848 | 1.46E−05 | 1.39E−03 | −0.091 |
| 28929 | FOXRED1 | chr11 | + | 126274926 | 126275021 | 126275326 | 126275428 | 126273335 | 126273454 | 126275793 | 126275870 | 2.62E−05 | 2.21E−03 | −0.092 |
| 34083 | DICER1-AS1 | chr14 | + | 95158192 | 95158234 | 95158875 | 95159035 | 95157759 | 95157858 | 95179503 | 95179925 | 1.17E−10 | 1.06E−07 | −0.092 |
| 39781 | CRBN | chr3 | − | 3154746 | 3154831 | 3156218 | 3156281 | 3153959 | 3154075 | 3167633 | 3167793 | 1.88E−04 | 9.88E−03 | −0.092 |
| 14977 | PACC1 | chr1 | − | 212375192 | 212375300 | 212379894 | 212380037 | 212363930 | 212365376 | 212385273 | 212385425 | 3.58E−04 | 1.56E−02 | −0.093 |
| 27815 | SPATA13 | chr13 | + | 24284134 | 24284271 | 24286213 | 24286393 | 24251717 | 24251862 | 24286764 | 24286950 | 2.15E−04 | 1.09E−02 | −0.093 |
| 3803 | ABCB7 | chrX | − | 75076521 | 75076654 | 75098941 | 75099061 | 75075361 | 75075630 | 75112885 | 75112972 | 1.92E−03 | 4.87E−02 | −0.094 |
| 13836 | DENND4B | chr1 | − | 153933196 | 153933319 | 153933482 | 153933871 | 153932860 | 153933030 | 153934134 | 153934302 | 4.94E−04 | 1.97E−02 | −0.094 |
| 28647 | JAK2 | chr9 | + | 4985939 | 4986022 | 5021962 | 5022213 | 498527 | 4985630 | 5029782 | 5029906 | 6.86E−05 | 4.58E−03 | −0.095 |
| 34025 | N4BP2L1 | chr13 | − | 32404320 | 32404397 | 32407249 | 32407338 | 32400722 | 32403200 | 32407644 | 32407772 | 3.87E−04 | 1.65E−02 | −0.095 |
| 33393 | IMMP1L | chr11 | − | 31460625 | 31460714 | 31473723 | 31473786 | 31456259 | 31456386 | 31509518 | 31509594 | 2.98E−06 | 4.12E−04 | −0.096 |
| 34258 | TBC1D2B | chr15 | − | 78044899 | 78045068 | 78054033 | 78054187 | 78030006 | 78030170 | 78077292 | 78077652 | 1.65E−03 | 4.42E−02 | −0.096 |
| 9231 | ATF7IP | chr12 | + | 1442390 | 14425473 | 14434339 | 14434423 | 14365675 | 14365827 | 14436105 | 14436251 | 9.69E−04 | 3.05E−02 | −0.097 |
| 27856 | SLBP | chr4 | − | 1700010 | 1700091 | 1703595 | 1703700 | 1699563 | 1699701 | 1711873 | 1711990 | 9.08E−05 | 5.70E−03 | −0.098 |
| 32188 | ARHGAP19 | chr10 | − | 97256317 | 97256404 | 97259401 | 97259628 | 97246271 | 97246337 | 97264825 | 97264906 | 1.22E−04 | 7.09E−03 | −0.098 |
| 5446 | POLL | chr10 | − | 101582762 | 101582891 | 101587245 | 101587406 | 101580247 | 101580416 | 101588203 | 101588232 | 5.87E−04 | 2.21E−02 | −0.099 |
| 14875 | IL15 | chr4 | + | 141656185 | 141656307 | 141719365 | 141719476 | 141636601 | 141637224 | 141720468 | 141720566 | 2.34E−07 | 5.28E−05 | −0.099 |
| 15441 | RIN3 | chr14 | + | 92555750 | 92555955 | 92615406 | 92615479 | 92513780 | 92513976 | 92641237 | 92641329 | 5.40E−04 | 2.09E−02 | −0.099 |
| 18552 | PXN | chr12 | − | 120216272 | 120216581 | 120219206 | 120220091 | 120215559 | 120215661 | 120221622 | 120221758 | 2.77E−05 | 2.31E−03 | −0.099 |
| 33749 | PLXDC1 | chr17 | − | 39109247 | 39109391 | 39139653 | 39139832 | 39108903 | 39108973 | 39151361 | 39151519 | 2.26E−04 | 1.13E−02 | −0.099 |
| 34026 | N4BP2L1 | chr13 | − | 32404320 | 32404397 | 32407644 | 32407772 | 32400722 | 32403200 | 32427903 | 32428130 | 1.64E−03 | 4.40E−02 | −0.099 |
| 38811 | PI4KAP2 | chr22 | − | 21487871 | 21488023 | 21491993 | 21492094 | 21487564 | 21487672 | 21517324 | 21517520 | 3.39E−06 | 4.52E−04 | −0.099 |
| 42416 | SUZ12P1 | chr17 | + | 30734896 | 30734943 | 30735032 | 30735097 | 30709607 | 30709811 | 30743300 | 30743369 | 3.26E−14 | 7.90E−11 | −0.099 |
| 22459 | SCAF8 | chr6 | + | 154778000 | 154778045 | 154787860 | 154788022 | 154773988 | 154774072 | 154792822 | 154792858 | 1.25E−04 | 7.18E−03 | −0.1 |
| 38017 | GTF2IRD2B | chr7 | + | 75112396 | 75112535 | 75120890 | 75121010 | 75108959 | 75109063 | 75123135 | 75123319 | 5.91E−04 | 2.22E−02 | −0.1 |
| 5160 | SENP6 | chr6 | + | 75623899 | 75623960 | 75634706 | 75634811 | 75621531 | 75621625 | 75640683 | 75640704 | 1.65E−03 | 4.42E−02 | −0.101 |
| 10848 | DNMT3A | chr2 | − | 25246619 | 25246776 | 25247590 | 25247749 | 25246159 | 25246309 | 25248036 | 25248252 | 1.29E−04 | 7.31E−03 | −0.101 |
| 35327 | DCP1B | chr12 | − | 1993263 | 1993391 | 1997934 | 1997975 | 1967843 | 1967910 | 2004281 | 2004427 | 9.97E−04 | 3.12E−02 | −0.101 |
| 26792 | TMEM39A | chr3 | − | 119452446 | 119452530 | 119458017 | 119458240 | 119446641 | 119447172 | 119461961 | 119462008 | 6.66E−04 | 2.43E−02 | −0.102 |
| 29301 | CBWD3 | chr9 | + | 68269599 | 68269722 | 68270301 | 68270518 | 68268933 | 68268978 | 68274881 | 68274938 | 3.89E−04 | 1.65E−02 | −0.102 |
| 37565 | RETSAT | chr2 | − | 85345974 | 85346094 | 85350779 | 85351021 | 85344593 | 85344732 | 85351679 | 85351862 | 1.21E−04 | 7.02E−03 | −0.102 |
| 8131 | DENND6A | chr3 | − | 57633264 | 57633354 | 57634557 | 57634622 | 57630924 | 57630978 | 57634703 | 57634769 | 3.94E−0 | 1.67E−02 | −0.103 |
| 20852 | ZSWIM7 | chr17 | − | 15977578 | 15977700 | 15977799 | 15977913 | 15976559 | 15977092 | 15978043 | 15978163 | 8.85E−04 | 2.89E−02 | −0.104 |
| 18925 | AK5 | chr1 | + | 77286940 | 77287127 | 77293792 | 77293960 | 77282018 | 77282373 | 77297558 | 77297728 | 3.30E−04 | 1.47E−02 | −0.105 |
| 36587 | MDM1 | chr12 | − | 68326656 | 68327021 | 68331106 | 68331221 | 68323072 | 68323240 | 68332227 | 68332286 | 1.50E−03 | 4.17E−02 | −0.105 |
| 4995 | ITGA6 | chr2 | + | 172467480 | 172467560 | 172470973 | 172471105 | 172465538 | 172465663 | 172474054 | 172474265 | 1.00E−03 | 3.14E−02 | −0.106 |
| 11244 | HDAC4 | chr2 | − | 239163802 | 239163923 | 239176412 | 239176563 | 239156651 | 239156773 | 239189832 | 239190077 | 4.34E−04 | 1.79E−02 | −0.108 |
| 19220 | SIRPB1 | chr20 | − | 1571719 | 1572037 | 1578337 | 1578694 | 1570804 | 1571137 | 1619868 | 1620009 | 8.21E−08 | 2.24E−05 | −0.108 |
| 41706 | ELMO2 | chr20 | − | 46380252 | 46380303 | 46383415 | 46383494 | 46375667 | 46375790 | 46386123 | 46386164 | 9.14E−04 | 2.94E−02 | −0.109 |
| 42372 | ABCA7 | chr19 | + | 1055096 | 1055351 | 1055906 | 1055939 | 1054779 | 1054878 | 1056065 | 1056243 | 6.36E−04 | 2.36E−02 | −0.109 |
| 5775 | HLA-B | chr6 | − | 31355106 | 31355223 | 31356687 | 31356957 | 31354632 | 31354665 | 31357085 | 31357179 | 3.86E−06 | 5.02E−04 | −0.111 |
| 11342 | KIAA0753 | chr17 | − | 6622881 | 6623097 | 6623508 | 6623571 | 6620787 | 6620998 | 6628116 | 6628741 | 6.88E−04 | 2.48E−02 | −0.111 |
| 22700 | MRNIP | chr5 | − | 179847977 | 179848066 | 179853196 | 179853276 | 179841906 | 179842064 | 179853377 | 179853437 | 5.72E−04 | 2.18E−02 | −0.111 |
| 28328 | RIOK1 | chr6 | + | 7395052 | 7395143 | 7396702 | 7396772 | 7393098 | 7393303 | 7398697 | 7398740 | 8.20E−04 | 2.76E−02 | −0.111 |
| 5776 | HLA-B | chr6 | − | 31355316 | 31355592 | 31356687 | 31356957 | 31355102 | 31355223 | 31357085 | 31357158 | 4.68E−08 | 1.42E−05 | −0.112 |
| 8409 | RNF144B | chr6 | + | 18399498 | 18399699 | 18427580 | 18427685 | 18387349 | 18387630 | 18439683 | 18439744 | 1.88E−03 | 4.82E−02 | −0.112 |
| 11391 | RAB35 | chr12 | − | 120099029 | 120099154 | 120108416 | 120108467 | 120096903 | 120097373 | 120116598 | 120116767 | 4.90E−05 | 3.58E−03 | −0.112 |
| 43731 | DOCK10 | chr2 | − | 224916694 | 224916784 | 224931548 | 224931668 | 224896294 | 224896377 | 225042251 | 225042442 | 6.35E−04 | 2.36E−02 | −0.112 |
| 36066 | FBXL4 | chr6 | − | 98917373 | 98917719 | 98934761 | 98934879 | 98905425 | 98905670 | 98947805 | 98947946 | 9.66E−04 | 3.05E−02 | −0.113 |
| 26671 | TMEM189-UBE2V1 | chr20 | − | 50130855 | 50130947 | 50143501 | 50143621 | 50129545 | 50129690 | 50153516 | 50153637 | 1.35E−03 | 3.86E−02 | −0.114 |
| 17250 | SEPTIN11 | chr4 | + | 76996424 | 76996539 | 77005600 | 77005796 | 76949702 | 76949930 | 77011734 | 77011921 | .16E−04 | 2.03E−02 | −0.115 |
| 14600 | RBM22 | chr5 | − | 150699241 | 150699271 | 150700443 | 150700497 | 150698498 | 150698631 | 150700931 | 150701046 | 1.13E−05 | 1.15E−03 | −0.116 |
| 30096 | BSCL2 | chr11 | − | 62702467 | 62702549 | 62705467 | 62705617 | 62694599 | 62694711 | 62706202 | 62706256 | 2.63E−04 | 1.25E−02 | −0.116 |
| 1916 | SNHG17 | chr20 | − | 38422091 | 38422241 | 38426418 | 38426503 | 38421005 | 38421098 | 38431040 | 38431112 | 1.00E−12 | 1.61E−09 | −0.117 |
| 8891 | SORBS3 | chr8 | + | 22566344 | 22566484 | 22566660 | 22566868 | 22565825 | 22565872 | 22567060 | 22567175 | 4.47E−04 | 1.83E−02 | −0.117 |
| 24359 | SPOPL | chr2 | + | 138550156 | 138550294 | 138550482 | 138550604 | 138501931 | 138502119 | 138550973 | 138551054 | 1.79E−03 | 4.67E−02 | −0.117 |
| 31230 | SLC7A6 | chr16 | + | 68274690 | 68275249 | 68287745 | 68287871 | 68266592 | 68266721 | 68290395 | 68290540 | 4.10E−04 | 1.72E−02 | −0.117 |
| 28384 | NDRG1 | chr8 | − | 133280231 | 133280267 | 133284248 | 133284329 | 133264546 | 133264652 | 133297133 | 133297256 | 3.12E−04 | 1.41E−02 | −0.118 |
| 31128 | LAIR1 | chr19 | − | 54361009 | 54361209 | 54364294 | 54364330 | 54360021 | 54360072 | 54364770 | 54364870 | 1.71E−04 | 9.18E−03 | −0.118 |
| 31130 | LAIR1 | chr19 | − | 54361009 | 54361243 | 54364294 | 54364330 | 54360021 | 54360072 | 54364770 | 54364931 | 1.53E−04 | 8.45E−03 | −0.119 |
| 33088 | ATL2 | chr2 | − | 38309378 | 38309506 | 38343267 | 38343512 | 38300271 | 38300328 | 38377142 | 38377273 | 1.28E−03 | 3.72E−02 | −0.119 |
| 22458 | SCAF8 | chr6 | + | 154773988 | 154774072 | 154787860 | 154788022 | 154733324 | 154733930 | 154792822 | 154792976 | 3.06E−04 | 1.38E−02 | −0.12 |
| 41542 | AC009061.2 | chr16 | + | 67484109 | 67484328 | 67492024 | 67492121 | 67481372 | 67481519 | 67504419 | 67505914 | .38E−04 | 1.49E−02 | −0.12 |
| 19302 | TMCO4 | chr1 | − | 19770541 | 19770569 | 19771307 | 19771482 | 19755633 | 19755766 | 19780579 | 19780764 | 9.46E−04 | 3.02E−02 | −0.121 |
| 30094 | BSCL2 | chr11 | − | 62702467 | 62702549 | 62705300 | 62705617 | 62694567 | 62694711 | 62706202 | 62706315 | 1.18E−04 | 6.94E−03 | −0.121 |
| 40521 | MAPK6 | chr15 | + | 52045829 | 52047015 | 52049992 | 52050137 | 52019218 | 52019376 | 52058632 | 52058797 | 3.61E−04 | 1.57E−02 | −0.121 |
| 18126 | PLSCR3 | chr17 | − | 7393466 | 7393509 | 7394103 | 7394267 | 7392883 | 7392952 | 7394476 | 7394545 | 4.95E−04 | 1.97E−02 | −0.122 |
| 7491 | FOXJ3 | chr1 | − | 42188736 | 42188928 | 42189302 | 42189404 | 42181916 | 42182024 | 42191302 | 42191719 | 8.50E−08 | 2.29E−05 | −0.124 |
| 43198 | SLC39A11 | chr17 | − | 72947730 | 72947875 | 73031555 | 73031714 | 72849633 | 72849804 | 73084807 | 73084846 | 4.89E−04 | 1.96E−02 | −0.124 |
| 6397 | PYROXD1 | chr12 | + | 21440367 | 21440448 | 21445346 | 21445466 | 21437614 | 21437814 | 21449562 | 21449691 | 1.62E−03 | 4.37E−02 | −0.125 |
| 8410 | RNF144B | chr6 | + | 18399498 | 18399699 | 18459606 | 18459751 | 18387349 | 18387630 | 18463290 | 18463380 | 1.27E−03 | 3.70E−02 | −0.125 |
| 19133 | TMEM220 | chr17 | − | 10725010 | 10725134 | 10726203 | 10726264 | 10723269 | 10723329 | 10729779 | 10730023 | 2.14E−04 | 1.09E−02 | −0.126 |
| 42526 | TADA2A | chr17 | + | 37442563 | 37442652 | 37444695 | 37444768 | 37440504 | 37440662 | 37458523 | 37458587 | 1.34E−03 | 3.86E−02 | −0.126 |
| 10056 | CYRIB | chr8 | − | 129912469 | 129912515 | 129948965 | 129949067 | 129904498 | 129904585 | 129970942 | 129970995 | 2.83E−06 | 3.96E−04 | −0.127 |
| 3736 | PEX1 | chr7 | − | 92518994 | 92519078 | 92522101 | 92522245 | 92518140 | 92518255 | 92528306 | 92528435 | 1.66E−03 | 4.42E−02 | −0.129 |
| 20897 | TSPAN2 | chr1 | − | 115058882 | 115058981 | 115072904 | 115073007 | 115057536 | 115057608 | 115089363 | 115089414 | 3.23E−04 | 1.45E−02 | −0.129 |
| 637 | TTLL12 | chr22 | − | 43180741 | 43180940 | 43182979 | 43183149 | 43179840 | 43180000 | 43186892 | 43187134 | 7.17E−05 | 4.74E−03 | −0.132 |
| 17843 | TMEM234 | chr1 | − | 32216383 | 32216517 | 32216888 | 32216947 | 32215846 | 32216281 | 32217258 | 32217351 | 9.00E−07 | 1.61E−04 | −0.136 |
| 35043 | BCL9L | chr11 | − | 118909913 | 118910015 | 118918825 | 118918879 | 118908416 | 118908655 | 118925237 | 118925453 | 1.62E−03 | 4.37E−02 | −0.136 |
| 19843 | AC008073.3 | chr2 | + | 24164197 | 24164335 | 24177801 | 24177870 | 24146929 | 24147086 | 24183284 | 24183372 | 2.45E−13 | 5.08E−10 | −0.137 |
| 27433 | GNB4 | chr3 | − | 179419398 | 179419505 | 179420888 | 179420927 | 179416492 | 179416556 | 179426143 | 179426242 | 8.68E−04 | 2.87E−02 | −0.138 |
| 20899 | TSPAN2 | chr1 | − | 115060463 | 115060538 | 115072904 | 115073007 | 115058882 | 115058981 | 115089363 | 115089414 | 8.80E−04 | 2.89E−02 | −0.139 |
| 17842 | TMEM234 | chr1 | − | 32215135 | 32215216 | 32216888 | 32216947 | 32214476 | 32215049 | 32217258 | 32217351 | 1.61E−04 | 8.79E−03 | −0.142 |
| 7449 | ERAP2 | chr5 | + | 96896731 | 96896863 | 96900120 | 96900245 | 96896372 | 96896504 | 96901505 | 96901681 | 1.37E−04 | 7.70E−03 | −0.143 |
| 7815 | ANKRD6 | chr6 | + | 89606006 | 89606105 | 89612271 | 89612370 | 89603028 | 89603127 | 89623409 | 89623544 | 1.88E−03 | 4.83E−02 | −0.144 |
| 6167 | DDX60L | chr4 | − | 168394460 | 168394617 | 168395958 | 168396127 | 168391539 | 168391640 | 168400825 | 168400978 | 2.02E−08 | 7.45E−06 | −0.147 |
| 21137 | GTF2H2B | chr5 | + | 70434178 | 70434266 | 70434914 | 70435022 | 70433869 | 70433960 | 70437530 | 70437594 | 3.38E−07 | 7.18E−05 | −0.148 |
| 34883 | SLC35F2 | chr11 | − | 107804717 | 107804770 | 107805358 | 107805515 | 107803000 | 107803155 | 107806716 | 107806876 | 1.05E−03 | 3.23E−02 | −0.148 |
| 34299 | TBCD | chr17 | + | 82763964 | 82764062 | 82766266 | 82766368 | 82756164 | 82756215 | 82768419 | 82768566 | 5.79E−05 | 4.07E−03 | −0.149 |
| 15900 | ZFR | chr5 | − | 32417647 | 32417792 | 32419820 | 32420103 | 32414968 | 32415187 | 32444228 | 32444328 | 1.89E−03 | 4.83E−02 | −0.156 |
| 18657 | PXYLP1 | chr3 | + | 141260122 | 141260254 | 141278341 | 141278500 | 141231783 | 141231911 | 141279377 | 141279504 | 6.15E−05 | 4.24E−03 | −0.158 |
| 11636 | SOS1 | chr2 | − | 39058672 | 39058804 | 39067627 | 39067753 | 39056701 | 39056866 | 39120335 | 39120450 | 8.02E−06 | 9.08E−04 | −0.16 |
| 1910 | SNHG17 | chr20 | − | 38422086 | 38422241 | 38425934 | 38426052 | 38421005 | 38421098 | 38426418 | 38426503 | 8.83E−04 | 2.89E−02 | −0.164 |
| 5757 | CA5B | chrX | + | 15721496 | 15721592 | 15745630 | 15745710 | 15688829 | 15688858 | 15749970 | 15750165 | 2.88E−04 | 1.33E−02 | −0.168 |
| 1915 | SNHG17 | chr20 | − | 38422091 | 38422241 | 38425934 | 38426052 | 38421005 | 38421098 | 38426418 | 38426503 | 6.29E−05 | 4.29E−03 | −0.171 |
| 39856 | ERC1 | chr12 | + | 1263033 | 1263165 | 1289851 | 1290012 | 1236768 | 1236904 | 1371832 | 1371977 | 1.49E−03 | 4.14E−02 | −0.175 |
| 10054 | CYRIB | chr8 | − | 129912469 | 129912515 | 129948965 | 129949067 | 129903311 | 129903350 | 129970942 | 129970995 | 5.35E−05 | 3.86E−03 | −0.176 |
| 36341 | RANBP10 | chr16 | − | 67772033 | 67772086 | 67805427 | 67805539 | 67744287 | 67744455 | 67806301 | 67806560 | 1.39E−05 | 1.34E−03 | −0.183 |
| 2514 | AC092070.2 | chr19 | + | 53200623 | 53200703 | 53204015 | 53204142 | 53197110 | 53197262 | 53210980 | 53211015 | 8.10E−04 | 2.74E−02 | −0.187 |
| 41472 | SETD6 | chr16 | + | 58516477 | 58516672 | 58516807 | 58516928 | 58516201 | 58516343 | 58518019 | 58518231 | 4.81E−04 | 1.94E−02 | −0.2 |
| 1297 | ITGB2 | chr21 | − | 44910283 | 44910372 | 44910724 | 44910785 | 44907986 | 44908182 | 44920820 | 44920896 | 1.28E−08 | 4.97E−06 | −0.209 |
| 11335 | CEP170 | chr1 | − | 243142366 | 243142463 | 243156220 | 243156347 | 243139936 | 243140107 | 243164283 | 243164494 | 1.76E−03 | 4.61E−02 | −0.21 |
| 1908 | SNHG17 | chr20 | − | 38422086 | 38422241 | 38422290 | 38422368 | 38421005 | 38421098 | 38426418 | 38426503 | 2.39E−04 | 1.18E−02 | −0.221 |
| 26614 | LRP8 | chr1 | − | 53262067 | 53262207 | 53264168 | 53264396 | 53260463 | 53260605 | 53266472 | 53266589 | 4.05E−05 | 3.11E−03 | −0.225 |
| 1913 | SNHG17 | chr20 | − | 38422091 | 38422241 | 38422290 | 38422368 | 38421005 | 38421098 | 38426418 | 38426503 | 9.64E−05 | 5.95E−03 | −0.233 |
| 16368 | ANKRD36B | chr2 | − | 97515731 | 97515945 | 97523325 | 97523467 | 97513179 | 97513363 | 97532310 | 97532384 | 1.64E−04 | 8.90E−03 | −0.254 |

TABLE 17
A3SS_WBC_FXSvsTD

| | | | | longExon | longExon | | | | | | | IncLevel |
|---|---|---|---|---|---|---|---|---|---|---|---|---|
| ID | GeneID | chr | strand | Start_Obase | End | shortES | shortEE | flankingES | flankingEE | PValue | FDR | Difference |
| 363 | PATZ1 | chr22 | − | 31328786 | 31328924 | 31328786 | 31328859 | 31335691 | 31335863 | 2.575E−04 | 9.757E−03 | −0.093 |
| 482 | TPTEP1 | chr22 | + | 16647604 | 16647785 | 16647662 | 16647785 | 16638578 | 16638740 | 1.397E−03 | 3.495E−02 | −0.149 |
| 656 | PABPC1L | chr20 | + | 44933056 | 44933185 | 44933071 | 44933185 | 44932681 | 44932713 | 1.938E−07 | 3.305E−05 | 0.079 |
| 679 | SNHG17 | chr20 | − | 38421005 | 38422368 | 38421005 | 38422241 | 38426418 | 38426503 | 2.330E−04 | 8.857E−03 | −0.17 |
| 699 | CPNE1 | chr20 | − | 35626566 | 35626803 | 35626566 | 35626800 | 35627279 | 35627413 | 3.721E−07 | 5.769E−05 | −0.086 |
| 700 | CPNE1 | chr20 | − | 35626566 | 35626826 | 35626566 | 35626800 | 35627279 | 35627413 | 1.322E−06 | 1.562E−04 | −0.12 |
| 909 | ZNF160 | chr19 | − | 53091412 | 53091529 | 53091412 | 53091497 | 53091636 | 53091720 | 1.670E−08 | 4.886E−06 | −0.052 |
| 991 | XAF1 | chr17 | + | 6759255 | 6759718 | 6759661 | 6759718 | 6758088 | 6758224 | 2.147E−08 | 5.852E−06 | 0.084 |
| 1004 | LSM7 | chr19 | − | 2324124 | 2324196 | 2324124 | 2324192 | 2325979 | 2326139 | 4.093E−04 | 1.373E−02 | −0.055 |
| 1251 | POLR2J3 | chr7 | − | 102543524 | 102543702 | 102543524 | 102543661 | 102544705 | 102544776 | 6.028E−05 | 3.256E−03 | 0.252 |
| 1347 | TSNARE1 | chr8 | − | 142315002 | 142315092 | 142315002 | 142315089 | 142318543 | 142318634 | 1.271E−03 | 3.293E−02 | 0.089 |
| 1364 | THADA | chr2 | − | 43551788 | 43551925 | 43551788 | 43551922 | 43552203 | 43552339 | 6.894E−14 | 1.372E−10 | −0.065 |
| 1380 | TRIM73 | chr7 | + | 75405181 | 75405605 | 75405184 | 75405605 | 75404838 | 75405069 | 6.331E−09 | 2.519E−06 | −0.149 |
| 1407 | AEBP1 | chr7 | + | 44109656 | 44110124 | 44110014 | 44110124 | 44109287 | 44109341 | 3.250E−08 | 7.608E−06 | 0.068 |
| 2043 | TBC1D7 | chr6 | − | 13326786 | 13326963 | 13326786 | 13326906 | 13328455 | 13328531 | 1.615E−03 | 3.864E−02 | 0.117 |
| 2046 | DDX60L | chr4 | − | 168395958 | 168396127 | 168395958 | 168396124 | 168400825 | 168400978 | 2.767E−08 | 6.740E−06 | −0.204 |
| 2196 | ASNS | chr7 | − | 97854579 | 97854754 | 97854579 | 97854680 | 97856689 | 97856816 | 9.989E−05 | 4.517E−03 | 0.062 |
| 2324 | MTSS1 | chr8 | − | 124556231 | 124556405 | 124556231 | 124556354 | 124562781 | 124562992 | 6.013E−04 | 1.860E−02 | 0.1 |
| 2349 | MZB1 | chr5 | − | 139388460 | 139388667 | 139388460 | 139388585 | 139389679 | 139389913 | 3.456E−05 | 2.053E−03 | 0.052 |
| 2384 | SEPTIN8 | chr5 | − | 132763705 | 132763892 | 132763705 | 132763886 | 132764223 | 132764419 | 7.908E−06 | 6.170E−04 | 0.134 |
| 2612 | PPWD1 | chr5 | + | 65569631 | 65569732 | 65569636 | 65569732 | 65568917 | 65568994 | 1.412E−03 | 3.512E−02 | 0.062 |
| 2703 | TMEM116 | chr12 | − | 111932585 | 111932731 | 111932585 | 111932659 | 111937159 | 111937243 | 3.981E−05 | 2.296E−03 | 0.097 |
| 2706 | TMEM116 | chr12 | − | 111933885 | 111936830 | 111933885 | 111934030 | 111938160 | 111938210 | 8.691E−06 | 6.598E−04 | 0.118 |
| 2778 | BRD9 | chr5 | − | 889586 | 889766 | 889586 | 889647 | 891154 | 891287 | 8.609E−06 | 6.588E−04 | 0.062 |
| 2783 | SDHA | chr5 | + | 254357 | 254506 | 254392 | 254506 | 240357 | 240476 | 2.137E−03 | 4.777E−02 | −0.156 |
| 3279 | IKBKG | chrX | + | 154558531 | 154558650 | 154558534 | 154558650 | 154556164 | 154556376 | 8.237E−05 | 3.933E−03 | −0.118 |
| 3301 | ZNF707 | chr8 | + | 143691596 | 143691713 | 143691599 | 143691713 | 143691072 | 143691232 | 1.985E−04 | 7.794E−03 | 0.104 |
| 3349 | CEP131 | chr17 | − | 81191192 | 81191350 | 81191192 | 81191335 | 81192317 | 81192392 | 2.848E−04 | 1.043E−02 | 0.103 |
| 3490 | DXO | chr6 | − | 31970342 | 31970478 | 31970342 | 31970378 | 31970605 | 31972252 | 3.220E−07 | 5.267E−05 | −0.054 |
| 3633 | TAFA2 | chr12 | − | 62258762 | 62258910 | 62258762 | 62258842 | 62260009 | 62260170 | 1.324E−03 | 3.378E−02 | −0.102 |
| 4002 | TPRG1 | chr3 | + | 189150655 | 189151136 | 189150903 | 189151136 | 189147583 | 189147647 | 8.847E−06 | 6.642E−04 | 0.07 |
| 4183 | DLG1 | chr3 | − | 197076585 | 197076685 | 197076585 | 197076682 | 197081050 | 197081117 | 1.456E−03 | 3.592E−02 | −0.092 |
| 4667 | GGT1 | chr22 | + | 24627409 | 24627619 | 24627431 | 24627619 | 24623779 | 24623916 | 6.307E−04 | 1.930E−02 | 0.059 |
| 4697 | SRGAP2 | chr1 | + | 206401420 | 206401645 | 206401423 | 206401645 | 206393544 | 206393673 | 3.538E−05 | 2.071E−03 | −0.203 |
| 4710 | TMEM161B-AS1 | chr5 | + | 88287435 | 88287622 | 88287438 | 88287622 | 88282714 | 88282866 | 2.697E−07 | 4.472E−05 | 0.062 |
| 4795 | HLA-C | chr6 | − | 31269492 | 31269543 | 31269492 | 31269525 | 31270209 | 31270485 | 3.391E−08 | 7.785E−06 | 0.056 |
| 4875 | CCDC163 | chr1 | − | 45495417 | 45495545 | 45495417 | 45495473 | 45496555 | 45496623 | 1.368E−03 | 3.459E−02 | 0.105 |
| 4877 | NABP1 | chr2 | + | 191683289 | 191683804 | 191683728 | 191683804 | 191681945 | 191682017 | 1.738E−03 | 4.077E−02 | 0.093 |
| 5259 | RNF181 | chr2 | + | 85596952 | 85597178 | 85597103 | 85597178 | 85596518 | 85596649 | 2.724E−05 | 1.690E−03 | −0.101 |
| 5644 | CENPT | chr16 | − | 67828473 | 67828605 | 67828473 | 67828559 | 67828666 | 67828843 | 2.587E−04 | 9.773E−03 | 0.061 |
| 5657 | KHDC4 | chr1 | − | 155916624 | 155916737 | 155916624 | 155916709 | 155917498 | 155917672 | 1.718E−03 | 4.037E−02 | −0.066 |
| 6027 | ZNF7 | chr8 | + | 144837390 | 144837507 | 144837393 | 144837507 | 144829477 | 144829604 | 2.617E−08 | 6.740E−06 | 0.068 |
| 6074 | DCAF11 | chr14 | + | 24115514 | 24115726 | 24115542 | 24115726 | 24114776 | 24114924 | 1.537E−03 | 3.706E−02 | 0.106 |
| 6270 | AK5 | chr1 | + | 77293792 | 77293960 | 77293864 | 77293960 | 77286940 | 77287127 | 1.553E−03 | 3.737E−02 | −0.074 |
| 6289 | ARRB2 | chr17 | + | 4715150 | 4715336 | 4715199 | 4715336 | 4715012 | 4715043 | 1.204E−03 | 3.130E−02 | 0.091 |
| 6563 | ARHGAP25 | chr2 | + | 68807272 | 68807480 | 68807275 | 68807480 | 68782232 | 68782320 | 1.005E−03 | 2.745E−02 | 0.081 |
| 6836 | AHSA2P | chr2 | + | 61178980 | 61179171 | 61178985 | 61179171 | 61177417 | 61178384 | 1.211E−05 | 8.604E−04 | −0.056 |
| 7058 | AZIN1 | chr8 | − | 102849998 | 102850165 | 102849998 | 102850144 | 102858012 | 102858150 | 5.316E−06 | 4.736E−04 | 0.056 |
| 7191 | ZNF540 | chr19 | + | 37601006 | 37601105 | 37601009 | 37601105 | 37599625 | 37599752 | 1.179E−03 | 3.073E−02 | −0.095 |
| 7317 | MFSD9 | chr2 | − | 102723699 | 102723909 | 102723699 | 102723878 | 102731024 | 102731068 | 2.271E−06 | 2.421E−04 | −0.061 |
| 7781 | JKAMP | chr14 | + | 59486712 | 59486804 | 59486730 | 59486804 | 59484989 | 59485116 | 1.991E−03 | 4.528E−02 | 0.058 |
| 7996 | HARS2 | chr5 | + | 140693490 | 140693665 | 140693590 | 140693665 | 140691606 | 140691756 | 1.835E−03 | 4.253E−02 | 0.065 |
| 8019 | RNF32 | chr7 | + | 156676294 | 156677130 | 156676418 | 156677130 | 156675695 | 156675863 | 2.109E−06 | 2.268E−04 | 0.054 |
| 8730 | BPHL | chr6 | + | 3138040 | 3140509 | 3140385 | 3140509 | 3137361 | 3137493 | 9.326E−04 | 2.613E−02 | 0.068 |
| 8964 | PPP6R3 | chr11 | + | 68587797 | 68588024 | 68587977 | 68588024 | 68575957 | 68576043 | 1.580E−11 | 1.451E−08 | −0.052 |
| 8967 | PPP6R3 | chr11 | + | 68587944 | 68588024 | 68587977 | 68588024 | 68575957 | 68576043 | 1.378E−11 | 1.371E−08 | −0.066 |
| 9028 | HELB | chr12 | + | 66324906 | 66325126 | 66324982 | 66325126 | 66323982 | 66324211 | 6.888E−06 | 5.593E−04 | −0.058 |
| 9377 | GALT | chr9 | + | 34648047 | 34648171 | 34648091 | 34648171 | 34647831 | 34647961 | 1.400E−05 | 9.772E−04 | 0.131 |
| 9662 | ALAD | chr9 | − | 113393446 | 113393634 | 113393446 | 113393630 | 113401211 | 113401284 | 1.351E−03 | 3.433E−02 | 0.053 |
| 10048 | LAIR1 | chr19 | − | 54364294 | 54364486 | 54364294 | 54364330 | 54364770 | 54364931 | 1.516E−04 | 6.282E−03 | 0.053 |
| 10049 | LAIR1 | chr19 | − | 54364294 | 54364486 | 54364294 | 54364330 | 54365029 | 54365099 | 5.878E−04 | 1.832E−02 | 0.058 |
| 10193 | AC016394.2 | chr10 | + | 73253800 | 73254349 | 73253892 | 73254349 | 73252790 | 73253095 | 5.903E−05 | 3.218E−03 | 0.187 |
| 10205 | CTSB | chr8 | − | 11867253 | 11867526 | 11867253 | 11867391 | 11868000 | 11868089 | 1.022E−08 | 3.589E−06 | 0.105 |
| 10491 | NAP1L4 | chr11 | − | 2989128 | 2989271 | 2989128 | 2989197 | 2990908 | 2990992 | 5.697E−07 | 7.728E−05 | 0.067 |
| 10498 | ZNF195 | chr11 | − | 3361742 | 3368855 | 3361742 | 3361889 | 3370974 | 3371070 | 1.890E−05 | 1.247E−03 | 0.147 |
| 10541 | TCTN1 | chr12 | + | 110628766 | 110628918 | 110628796 | 110628918 | 110626361 | 110626492 | 1.680E−04 | 6.797E−03 | −0.067 |
| 10777 | PHB2 | chr12 | − | 6969955 | 6970068 | 6969955 | 6970063 | 6970195 | 6970280 | 3.965E−06 | 3.848E−04 | −0.154 |
| 10783 | PHF1 | chr6 | + | 33415234 | 33415329 | 33415260 | 33415329 | 33414724 | 33414829 | 4.419E−06 | 4.154E−04 | −0.101 |
| 11056 | ATG16L2 | chr11 | + | 72821198 | 72821741 | 72821547 | 72821741 | 72817755 | 72817855 | 6.675E−04 | 2.012E−02 | 0.07 |
| 11251 | TMEM25 | chr11 | + | 118532149 | 118532461 | 118532245 | 118532461 | 118531774 | 118531871 | 7.325E−04 | 2.138E−02 | −0.056 |
| 11458 | SPSB2 | chr12 | − | 6872237 | 6872997 | 6872237 | 6872986 | 6873245 | 6873336 | 7.668E−04 | 2.222E−02 | 0.097 |
| 11625 | YAF2 | chr12 | − | 42210547 | 42210680 | 42210547 | 42210619 | 42212443 | 42212484 | 9.647E−05 | 4.464E−03 | −0.067 |
| 11709 | MAP3K12 | chr12 | − | 53486946 | 53487155 | 53486946 | 53487145 | 53487254 | 53487428 | 6.237E−10 | 3.103E−07 | 0.149 |
| 11949 | ATXN2 | chr12 | − | 111510384 | 111510582 | 111510384 | 111510444 | 111513356 | 111513539 | 1.054E−04 | 4.693E−03 | −0.059 |
| 12038 | ARL6IP4 | chr12 | + | 122981570 | 122981879 | 122981594 | 122981879 | 122981128 | 122981266 | 2.026E−07 | 3.406E−05 | 0.059 |
| 12372 | TMEM273 | chr10 | − | 49165204 | 49165361 | 49165204 | 49165283 | 49165765 | 49165796 | 1.462E−03 | 3.598E−02 | −0.064 |
| 12471 | MPP5 | chr14 | + | 67279017 | 67279537 | 67279249 | 67279537 | 67269700 | 67269783 | 3.136E−10 | 1.628E−07 | 0.078 |
| 12520 | ALDH6A1 | chr14 | − | 74071194 | 74071660 | 74071194 | 74071497 | 74071895 | 74071974 | 6.790E−04 | 2.040E−02 | 0.087 |
| 12861 | LTK | chr15 | − | 41508068 | 41508236 | 41508068 | 41508221 | 41509030 | 41509129 | 2.785E−06 | 2.818E−04 | −0.065 |
| 13022 | FAM219B | chr15 | − | 74904981 | 74905059 | 74904981 | 74905039 | 74905153 | 74905231 | 5.182E−06 | 4.707E−04 | 0.134 |
| 13143 | MAN2A2 | chr15 | + | 90911366 | 90911550 | 90911415 | 90911550 | 90911170 | 90911238 | 6.973E−04 | 2.066E−02 | −0.053 |
| 13417 | PSTPIP1 | chr15 | + | 77027799 | 77027914 | 77027851 | 77027914 | 77026115 | 77026172 | 8.074E−04 | 2.323E−02 | 0.081 |
| 13584 | ZNF23 | chr16 | − | 71447596 | 71449885 | 71447596 | 71449882 | 71453242 | 71453350 | 1.154E−05 | 8.250E−04 | 0.054 |
| 13675 | SPG7 | chr16 | + | 89548979 | 89549336 | 89549080 | 89549336 | 89548002 | 89548113 | 3.097E−08 | 7.394E−06 | 0.108 |
| 13787 | GABBR1 | chr6 | − | 29629086 | 29629247 | 29629086 | 29629107 | 29630521 | 29630643 | 5.945E−04 | 1.846E−02 | 0.072 |
| 14009 | MLX | chr17 | + | 42569203 | 42569606 | 42569506 | 42569606 | 42567618 | 42567655 | 1.887E−15 | 4.506E−12 | 0.064 |
| 14021 | BRCA1 | chr17 | − | 43076487 | 43076614 | 43076487 | 43076611 | 43082403 | 43082575 | 2.100E−05 | 1.348E−03 | 0.233 |
| 14113 | TSPOAP1-AS1 | chr17 | + | 58351734 | 58351914 | 58351743 | 58351914 | 58337447 | 58337679 | 6.160E−05 | 3.303E−03 | −0.108 |
| 14185 | DPH2 | chr1 | + | 43970905 | 43971189 | 43970965 | 43971189 | 43970595 | 43970708 | 1.145E−03 | 3.011E−02 | 0.08 |
| 14321 | MPPE1 | chr18 | − | 11885675 | 11885912 | 11885675 | 11885816 | 11886916 | 11887025 | 4.754E−04 | 1.559E−02 | 0.073 |
| 14371 | CCDC191 | chr3 | − | 114042702 | 114042846 | 114042702 | 114042841 | 114046590 | 114046732 | 6.391E−08 | 1.387E−05 | −0.065 |
| 14648 | PCBP1-AS1 | chr2 | − | 70048777 | 70050252 | 70048777 | 70048957 | 70051202 | 70051305 | 1.496E−03 | 3.638E−02 | 0.083 |
| 14655 | PCBP1-AS1 | chr2 | − | 70053730 | 70053812 | 70053730 | 70053792 | 70055713 | 70055910 | 2.310E−04 | 8.857E−03 | 0.067 |
| 14656 | PCBP1-AS1 | chr2 | − | 70053730 | 70053815 | 70053730 | 70053792 | 70055713 | 70055910 | 4.592E−04 | 1.518E−02 | 0.061 |
| 14685 | VPS33B | chr15 | − | 91016962 | 91017064 | 91016962 | 91017024 | 91017804 | 91017885 | 3.163E−06 | 3.147E−04 | −0.053 |
| 14753 | UNC50 | chr2 | + | 98618145 | 98618515 | 98618167 | 98618515 | 98610774 | 98610895 | 5.877E−05 | 3.218E−03 | −0.083 |
| 14829 | ZNF345 | chr19 | + | 36851727 | 36851904 | 36851829 | 36851904 | 36850929 | 36850968 | 4.124E−05 | 2.352E−03 | 0.105 |

TABLE 18
A5SS_WBC_FXSvsTD

| | | | | longExon | longExon | | | | | | | IncLevel |
|---|---|---|---|---|---|---|---|---|---|---|---|---|
| ID | GeneID | chr | strand | Start_Obase | End | shortES | shortEE | flankingES | flankingEE | PValue | FDR | Difference |
| 124 | PQBP1 | chrX | + | 48897911 | 48898093 | 48897911 | 48898082 | 48898491 | 48898576 | 1.814E−06 | 1.997E−04 | −0.203 |
| 433 | PIGT | chr20 | + | 45416516 | 45418979 | 45416516 | 45416694 | 45419294 | 45419395 | 5.964E−04 | 2.024E−02 | −0.057 |
| 444 | TTPAL | chr20 | + | 44484336 | 44484530 | 44484336 | 44484428 | 44486595 | 44486706 | 5.212E−04 | 1.861E−02 | −0.05 |
| 689 | FNTA | chr8 | + | 43077215 | 43080441 | 43077215 | 43077364 | 43084709 | 43084881 | 2.519E−05 | 1.668E−03 | −0.075 |
| 779 | NEIL2 | chr8 | + | 11769709 | 11770335 | 11769709 | 11769791 | 11771445 | 11771585 | 3.929E−04 | 1.517E−02 | 0.181 |
| 797 | CEP152 | chr15 | − | 48741600 | 48741704 | 48741630 | 48741704 | 48738150 | 48739288 | 0.000E+00 | 0.000E+00 | 0.063 |
| 1082 | DEPDC5 | chr22 | + | 31797599 | 31797703 | 31797599 | 31797699 | 31798581 | 31798656 | 1.878E−09 | 8.995E−07 | −0.076 |
| 1267 | HLA-DMA | chr6 | − | 32949270 | 32949399 | 32949273 | 32949399 | 32948612 | 32948868 | 3.708E−06 | 3.471E−04 | −0.055 |
| 1283 | MICA | chr6 | + | 31399783 | 31400783 | 31399783 | 31400763 | 31410542 | 31410797 | 5.733E−04 | 1.979E−02 | 0.103 |
| 1350 | MSTO1 | chr1 | + | 155611215 | 155611355 | 155611215 | 155611291 | 155611548 | 155611603 | 4.273E−07 | 6.327E−05 | −0.068 |
| 1351 | MSTO1 | chr1 | + | 155611215 | 155611355 | 155611215 | 155611291 | 155611739 | 155611827 | 5.946E−05 | 3.509E−03 | 0.056 |
| 1353 | MSTO1 | chr1 | + | 155613656 | 155613766 | 155613656 | 155613733 | 155614058 | 155614935 | 1.432E−03 | 4.098E−02 | −0.153 |
| 1671 | ERAP2 | chr5 | + | 96909579 | 96909809 | 96909579 | 96909764 | 96912636 | 96912798 | 1.796E−05 | 1.367E−03 | 0.094 |
| 2241 | PARP2 | chr14 | + | 20344931 | 20345126 | 20344931 | 20345087 | 20345393 | 20345464 | 2.068E−05 | 1.490E−03 | 0.161 |
| 2634 | ZSCAN25 | chr7 | + | 99619560 | 99621574 | 99619560 | 99619993 | 99622548 | 99622640 | 4.730E−07 | 6.879E−05 | −0.089 |
| 2981 | HAGHL | chr16 | + | 728115 | 728424 | 728115 | 728233 | 728503 | 728604 | 1.079E−03 | 3.290E−02 | −0.072 |
| 3121 | BUD31 | chr7 | + | 99408968 | 99409245 | 99408968 | 99409004 | 99411063 | 99411186 | 3.127E−04 | 1.267E−02 | 0.095 |
| 3547 | FCRL1 | chr1 | − | 157797680 | 157797820 | 157797758 | 157797820 | 157797100 | 157797132 | 5.073E−04 | 1.836E−02 | 0.139 |
| 3578 | AL392172.1 | chr1 | + | 222836995 | 222837090 | 222836995 | 222837066 | 222837218 | 222837383 | 3.683E−04 | 1.442E−02 | 0.166 |
| 3642 | NDUFV2 | chr18 | + | 9124873 | 9124996 | 9124873 | 9124983 | 9134185 | 9134341 | 2.270E−12 | 3.082E−09 | 0.063 |
| 3815 | ERVK13-1 | chr16 | − | 2671023 | 2672570 | 2671594 | 2672570 | 2669410 | 2669561 | 1.533E−07 | 3.060E−05 | 0.061 |
| 3816 | ERVK13-1 | chr16 | − | 2671344 | 2672570 | 2671594 | 2672570 | 2669410 | 2669561 | 3.842E−07 | 5.794E−05 | 0.061 |
| 3993 | APTR | chr7 | − | 77695895 | 77696255 | 77696171 | 77696255 | 77685201 | 77685328 | 7.684E−04 | 2.493E−02 | −0.057 |
| 4345 | KLRD1 | chr12 | + | 10311463 | 10311619 | 10311463 | 10311615 | 10313406 | 10313513 | 1.493E−05 | 1.169E−03 | −0.057 |
| 4583 | IRF3 | chr19 | − | 49665483 | 49665857 | 49665630 | 49665857 | 49664673 | 49664846 | 2.417E−04 | 1.060E−02 | 0.102 |
| 4596 | CUL7 | chr6 | − | 43046510 | 43047107 | 43046879 | 43047107 | 43046235 | 43046407 | 1.645E−03 | 4.549E−02 | −0.104 |
| 4732 | ZDHHC3 | chr3 | − | 44959126 | 44959460 | 44959130 | 44959460 | 44933887 | 44933984 | 6.471E−07 | 9.086E−05 | −0.198 |
| 4733 | ZDHHC3 | chr3 | − | 44959126 | 44959460 | 44959130 | 44959460 | 44958587 | 44958685 | 1.299E−03 | 3.792E−02 | 0.204 |
| 4848 | RBIS | chr8 | − | 85219235 | 85219363 | 85219322 | 85219363 | 85217385 | 85217502 | 1.209E−03 | 3.607E−02 | 0.181 |
| 5006 | PCBP4 | chr3 | − | 51961748 | 51962088 | 51961940 | 51962088 | 51961159 | 51961304 | 1.276E−03 | 3.743E−02 | 0.113 |
| 5131 | ATP5F1A | chr18 | − | 46094979 | 46095131 | 46095052 | 46095131 | 46093179 | 46093362 | 9.621E−08 | 2.304E−05 | 0.063 |
| 5241 | LUCAT1 | chr5 | − | 91313961 | 91314404 | 91314079 | 91314404 | 91313637 | 91313777 | 4.792E−04 | 1.758E−02 | 0.073 |
| 5401 | CCDC14 | chr3 | − | 123946802 | 123947319 | 123947279 | 123947319 | 123944848 | 123944990 | 4.595E−04 | 1.701E−02 | 0.052 |
| 5514 | UBE2I | chr16 | + | 1312346 | 1312603 | 1312346 | 1312558 | 1314019 | 1314096 | 2.004E−05 | 1.457E−03 | −0.155 |
| 5525 | ELOA-AS1 | chr1 | − | 23772267 | 23772426 | 23772271 | 23772426 | 23772110 | 23772166 | 1.603E−03 | 4.469E−02 | 0.177 |
| 5765 | SLC25A37 | chr8 | + | 23566107 | 23566813 | 23566107 | 23566336 | 23568321 | 23568378 | 8.743E−05 | 4.844E−03 | 0.058 |
| 5869 | AGA | chr4 | − | 177436275 | 177436351 | 177436297 | 177436351 | 177434439 | 177434481 | 6.320E−04 | 2.118E−02 | −0.062 |
| 6030 | CNBP | chr3 | − | 129171651 | 129171771 | 129171654 | 129171771 | 129171445 | 129171508 | 6.977E−08 | 1.776E−05 | −0.059 |
| 6774 | PPIEL | chr1 | − | 39529015 | 39529164 | 39529054 | 39529164 | 39522279 | 39522505 | 1.308E−05 | 1.035E−03 | 0.066 |
| 6952 | AC022400.7 | chr10 | − | 73801339 | 73802776 | 73802673 | 73802776 | 73797821 | 73798059 | 1.069E−03 | 3.271E−02 | 0.073 |
| 7048 | MRPL43 | chr10 | − | 100986686 | 100986975 | 100986748 | 100986975 | 100983754 | 100983817 | 1.186E−09 | 6.580E−07 | −0.142 |
| 7049 | MRPL43 | chr10 | − | 100986686 | 100986975 | 100986748 | 100986975 | 100984033 | 100984074 | 2.626E−06 | 2.608E−04 | −0.108 |
| 7261 | SERGEF | chr11 | − | 18010070 | 18010139 | 18010074 | 18010139 | 18007940 | 18008076 | 9.450E−05 | 5.200E−03 | −0.159 |
| 7347 | STAT2 | chr12 | − | 56349344 | 56349509 | 56349425 | 56349509 | 56349162 | 56349261 | 7.153E−05 | 4.103E−03 | 0.053 |
| 7561 | ULK3 | chr15 | − | 74837750 | 74837798 | 74837756 | 74837798 | 74837368 | 74837435 | 1.847E−07 | 3.419E−05 | −0.216 |
| 7726 | SLC6A12 | chr12 | − | 209772 | 210043 | 209890 | 210043 | 204563 | 204698 | 3.174E−05 | 2.052E−03 | 0.131 |
| 7976 | ANXA2 | chr15 | − | 60396150 | 60396427 | 60396279 | 60396427 | 60386027 | 60386086 | 2.116E−04 | 9.763E−03 | 0.125 |
| 8022 | ITGB7 | chr12 | − | 53201117 | 53201194 | 53201146 | 53201194 | 53200242 | 53200446 | 3.569E−12 | 3.685E−09 | 0.07 |
| 8039 | TM6SF1 | chr15 | + | 83119577 | 83119734 | 83119577 | 83119681 | 83120048 | 83120086 | 2.421E−04 | 1.060E−02 | 0.059 |
| 8274 | ARL6IP4 | chr12 | + | 122981128 | 122981299 | 122981128 | 122981266 | 122981570 | 122981879 | 2.529E−06 | 2.575E−04 | −0.052 |
| 8300 | DDX51 | chr12 | − | 132141102 | 132141420 | 132141274 | 132141420 | 132140830 | 132141020 | 1.262E−06 | 1.579E−04 | 0.081 |
| 8418 | UBAC2-AS1 | chr13 | − | 99200365 | 99200710 | 99200637 | 99200710 | 99196376 | 99197802 | 1.220E−05 | 9.837E−04 | −0.1 |
| 8426 | PXN-AS1 | chr12 | + | 120206544 | 120206712 | 120206544 | 120206621 | 120209838 | 120209934 | 1.727E−04 | 8.322E−03 | 0.058 |
| 8449 | APEX1 | chr14 | + | 20455577 | 20455773 | 20455577 | 20455703 | 20456667 | 20456860 | 1.489E−04 | 7.485E−03 | 0.074 |
| 8600 | NEK3 | chr13 | − | 52143914 | 52143987 | 52143915 | 52143987 | 52136799 | 52136902 | 8.795E−09 | 3.256E−06 | 0.277 |
| 8601 | NEK3 | chr13 | − | 52143914 | 52143987 | 52143915 | 52143987 | 52141019 | 52141069 | 1.920E−05 | 1.422E−03 | 0.251 |
| 8780 | TPM1 | chr15 | + | 63060868 | 63061273 | 63060868 | 63060939 | 63061712 | 63061788 | 3.759E−06 | 3.479E−04 | 0.066 |
| 9058 | SMPD1 | chr11 | + | 6391383 | 6392196 | 6391383 | 6392156 | 6393215 | 6393387 | 3.024E−07 | 5.026E−05 | 0.06 |
| 9240 | UBC | chr12 | − | 124913320 | 124913774 | 124913495 | 124913774 | 124913034 | 124913267 | 1.497E−07 | 3.060E−05 | −0.053 |
| 9437 | DVL2 | chr17 | − | 7230045 | 7230155 | 7230057 | 7230155 | 7229807 | 7229943 | 1.446E−06 | 1.707E−04 | 0.119 |
| 9733 | DPH2 | chr1 | + | 43970595 | 43970817 | 43970595 | 43970708 | 43970965 | 43971189 | 1.388E−03 | 4.007E−02 | 0.083 |
| 10198 | AC243960.1 | chr19 | + | 41551179 | 41551494 | 41551179 | 41551302 | 41551752 | 41551828 | 3.620E−12 | 3.685E−09 | 0.065 |

TABLE 19
RI_WBC_FXSvsTD

| | | | | riExon | riExon | upstream |
|---|---|---|---|---|---|---|
| ID | GeneID | chr | strand | Start_0base | End | ES |
| 2112 | GPATCH4 | chr1 | − | 156596080 | 156596478 | 156596080 |
| 6377 | CAPN3 | chr15 | + | 42409930 | 42410496 | 42409930 |
| 4280 | SUN1 | chr7 | + | 852608 | 852952 | 852608 |
| 2854 | ZNF7 | chr8 | + | 144829042 | 144829604 | 144829042 |
| 956 | ATAT1 | chr6 | + | 30645894 | 30646109 | 30645894 |
| 6880 | PER1 | chr17 | − | 8146896 | 8147381 | 8146896 |
| 991 | MSTO1 | chr1 | + | 155611215 | 155611603 | 155611215 |
| 4675 | DHRS4L2 | chr14 | + | 24000862 | 24001084 | 24000862 |
| 3464 | RELA-DT | chr11 | + | 65663426 | 65665543 | 65663426 |
| 5238 | TM7SF2 | chr11 | + | 65112810 | 65113414 | 65112810 |
| 2533 | DERL3 | chr22 | − | 23834502 | 23837154 | 23834502 |
| 3692 | ASB16-AS1 | chr17 | − | 44181622 | 44183093 | 44181622 |
| 6333 | FAN1 | chr15 | + | 30925788 | 30928656 | 30925788 |
| 5791 | ZFC3H1 | chr12 | − | 71656342 | 71657301 | 71656342 |
| 1952 | NRBP2 | chr8 | − | 143839508 | 143839825 | 143839508 |
| 6231 | OGFOD2 | chr12 | + | 122975810 | 122976767 | 122975810 |
| 4279 | SUN1 | chr7 | + | 852608 | 852952 | 852608 |
| 5676 | AAAS | chr12 | − | 53315093 | 53315782 | 53315093 |
| 5999 | LILRA1 | chr19 | + | 54594196 | 54594476 | 54594196 |
| 4158 | BPHL | chr6 | + | 3138040 | 3140509 | 3138040 |
| 5920 | DDX51 | chr12 | − | 132140830 | 132141420 | 132140830 |
| 7297 | ZNF266 | chr19 | − | 9418761 | 9419299 | 9418761 |
| 2287 | WDR54 | chr2 | + | 74423318 | 74423982 | 74423318 |
| 6335 | BCS1L | chr2 | + | 218662196 | 218662679 | 218662196 |
| 5956 | FCSK | chr16 | + | 70466881 | 70467471 | 70466881 |
| 814 | SEC31B | chr10 | − | 100505360 | 100506201 | 100505360 |
| 4875 | POLG | chr15 | − | 89317938 | 89318749 | 89317938 |
| 505 | CD3E | chr11 | + | 118312152 | 118313874 | 118312152 |
| 3884 | CD19 | chr16 | + | 28932345 | 28933114 | 28932345 |
| 6388 | TUBGCP4 | chr15 | + | 43401715 | 43403799 | 43401715 |
| 5776 | AVIL | chr12 | − | 57807330 | 57807727 | 57807330 |
| 451 | ZNF160 | chr19 | − | 53091412 | 53091720 | 53091412 |
| 6105 | MINK1 | chr17 | + | 4889646 | 4890735 | 4889646 |
| 1075 | TTC16 | chr9 | + | 127724119 | 127724897 | 127724119 |
| 5044 | ING4 | chr12 | − | 6652661 | 6653050 | 6652661 |
| 2645 | FCGR2B | chr1 | + | 161673959 | 161677365 | 161673959 |
| 512 | GINS4 | chr8 | + | 41541808 | 41545030 | 41541808 |
| 1558 | C1R | chr12 | − | 7091451 | 7092441 | 7091451 |
| 6474 | TRIM27 | chr6 | − | 28903001 | 28908840 | 28903001 |
| 5134 | RABEPK | chr9 | + | 125200541 | 125200906 | 125200541 |
| 6881 | CTC1 | chr17 | − | 8229141 | 8229446 | 8229141 |
| 6467 | MAN2C1 | chr15 | − | 75359058 | 75359425 | 75359058 |
| 4769 | RASGRP4 | chr19 | − | 38419859 | 38420262 | 38419859 |
| 6555 | WDR24 | chr16 | − | 685868 | 686186 | 685868 |
| 7458 | RINL | chr19 | − | 38876330 | 38876781 | 38876330 |
| 1877 | ZSCAN25 | chr7 | + | 99619560 | 99621574 | 99619560 |
| 6230 | OGFOD2 | chr12 | + | 122975810 | 122976767 | 122975810 |
| 2817 | C1orf174 | chr1 | − | 3890568 | 3892996 | 3890568 |
| 4726 | PAXX | chr9 | + | 136992639 | 136992974 | 136992639 |
| 2714 | LINC01128 | chr1 | + | 851926 | 852766 | 851926 |
| 98 | PRICKLE3 | chrX | − | 49177992 | 49178475 | 49177992 |
| 3273 | IRF3 | chr19 | − | 49664673 | 49665857 | 49664673 |
| 1466 | MUTYH | chr1 | − | 45332762 | 45333324 | 45332762 |
| 1387 | FAHD2A | chr2 | + | 95410526 | 95411026 | 95410526 |
| 4336 | RIOK1 | chr6 | + | 7402602 | 7402897 | 7402602 |
| 6191 | ZC3H14 | chr14 | + | 88609266 | 88609803 | 88609266 |
| 6738 | TMEM208 | chr16 | + | 67228796 | 67229278 | 67228796 |
| 3805 | HARS2 | chr5 | + | 140693490 | 140693665 | 140693490 |
| 5227 | MAP4K2 | chr11 | − | 64791908 | 64792422 | 64791908 |
| 3990 | WDR6 | chr3 | + | 49011634 | 49014293 | 49011634 |
| 454 | ZNF649 | chr19 | − | 51899797 | 51900294 | 51899797 |
| 1669 | NEPRO | chr3 | − | 113012733 | 113013403 | 113012733 |
| 5177 | BEST1 | chr11 | + | 61959891 | 61962893 | 61959891 |
| 5309 | THOC6 | chr16 | + | 3025923 | 3026288 | 3025923 |
| 5633 | PFKM | chr12 | + | 48134942 | 48135383 | 48134942 |
| 345 | ADA | chr20 | − | 44622828 | 44623078 | 44622828 |
| 6166 | IFT43 | chr14 | + | 76082294 | 76082692 | 76082294 |
| 3341 | PPIE | chr1 | + | 39741894 | 39743297 | 39741894 |
| 1480 | BTN3A3 | chr6 | + | 26443956 | 26445985 | 26443956 |
| 650 | SAMD9L | chr7 | − | 93145384 | 93146040 | 93145384 |
| 7170 | MPPE1 | chr18 | − | 11886498 | 11886778 | 11886498 |
| 4667 | IFFO1 | chr12 | − | 6548064 | 6548545 | 6548064 |
| 1759 | NAGK | chr2 | + | 71068649 | 71070586 | 71068649 |
| 7315 | CCDC159 | chr19 | + | 11353450 | 11353874 | 11353450 |
| 3316 | SAP30BP | chr17 | + | 75705610 | 75706092 | 75705610 |
| 3511 | MRNIP | chr5 | − | 179837275 | 179840959 | 179837275 |
| 6087 | ARHGAP9 | chr12 | − | 57476589 | 57476963 | 57476589 |
| 1342 | XPOT | chr12 | + | 64425037 | 64425457 | 64425037 |
| 2324 | P3H1 | chr1 | − | 42746374 | 42747412 | 42746374 |
| 6079 | REC8 | chr14 | + | 24172898 | 24173198 | 24172898 |
| 2196 | SCML4 | chr6 | − | 107744948 | 107746889 | 107744948 |
| 6015 | APEX1 | chr14 | + | 20455225 | 20455703 | 20455225 |
| 7124 | ENDOV | chr17 | + | 80415452 | 80415821 | 80415452 |
| 1943 | HMBS | chr11 | + | 119092124 | 119092523 | 119092124 |
| 3408 | BRPF1 | chr3 | + | 9745572 | 9745930 | 9745572 |
| 4440 | DHRS1 | chr14 | − | 24292183 | 24292784 | 24292183 |
| 4554 | NPHP3 | chr3 | − | 132684553 | 132686387 | 132684553 |
| 4628 | KLF4 | chr9 | − | 107487027 | 107488267 | 107487027 |
| 5685 | CALCOCO1 | chr12 | − | 53713700 | 53714241 | 53713700 |
| 6355 | RPAIN | chr17 | + | 5425970 | 5428211 | 5425970 |
| 1718 | CIRBP | chr19 | + | 1271980 | 1274440 | 1271980 |
| 2447 | KRIT1 | chr7 | − | 92235402 | 92236542 | 92235402 |
| 3163 | BSDC1 | chr1 | − | 32394079 | 32394425 | 32394079 |
| 4469 | GALT | chr9 | + | 34648333 | 34648894 | 34648333 |
| 6222 | TMEM150A | chr2 | − | 85601020 | 85601482 | 85601020 |
| 1130 | ZSWIM8 | chr10 | + | 73794146 | 73794639 | 73794146 |
| 1409 | DPH1 | chr17 | + | 2040217 | 2040605 | 2040217 |
| 7169 | TMEM205 | chr19 | − | 11345519 | 11346268 | 11345519 |
| 1349 | CARD8 | chr19 | − | 48232452 | 48234543 | 48232452 |
| 1569 | LFNG | chr7 | + | 2525218 | 2525567 | 2525218 |
| 3080 | NDUFAF7 | chr2 | + | 37241577 | 37242693 | 37241577 |
| 3717 | ARGLU1 | chr13 | − | 106557047 | 106559657 | 106557047 |
| 1050 | RNF44 | chr5 | − | 176529730 | 176530206 | 176529730 |
| 1951 | NRBP2 | chr8 | − | 143835819 | 143836030 | 143835819 |
| 2751 | ERVK13-1 | chr16 | − | 2669410 | 2672570 | 2669410 |
| 2853 | ZNF7 | chr8 | + | 144829042 | 144829604 | 144829042 |
| 990 | MSTO1 | chr1 | + | 155611215 | 155611603 | 155611215 |
| 2218 | MIB2 | chr1 | + | 1628272 | 1628722 | 1628272 |
| 6038 | TMEM147 | chr19 | + | 35546671 | 35547240 | 35546671 |
| 5988 | BBS1 | chr11 | + | 66515539 | 66515731 | 66515539 |
| 450 | ZNF160 | chr19 | − | 53091412 | 53091720 | 53091412 |
| 455 | ZNF577 | chr19 | − | 51886820 | 51887936 | 51886820 |
| 2270 | MFSD2A | chr1 | + | 39967627 | 39967916 | 39967627 |
| 5428 | VPS11 | chr11 | + | 119078172 | 119078997 | 119078172 |
| 5051 | PPOX | chr1 | + | 161170906 | 161171181 | 161170906 |
| 5810 | CDK16 | chrX | + | 47225964 | 47226703 | 47225964 |
| 2215 | MIB2 | chr1 | + | 1625545 | 1626754 | 1625545 |
| 5327 | PRPF40B | chr12 | + | 49642234 | 49642675 | 49642234 |
| 1174 | STARD5 | chr15 | − | 81322407 | 81324141 | 81322407 |
| 2216 | MIB2 | chr1 | + | 1626836 | 1627207 | 1626836 |
| 5811 | CDK16 | chrX | + | 47226993 | 47227223 | 47226993 |
| 5409 | ALG9 | chr11 | − | 111782194 | 111786520 | 111782194 |
| 4758 | PPIEL | chr1 | − | 39547578 | 39548951 | 39547578 |
| 1291 | HTRA2 | chr2 | + | 74531338 | 74531702 | 74531338 |
| 6466 | MAN2C1 | chr15 | − | 75358461 | 75358808 | 75358461 |
| 453 | ZNF528-AS1 | chr19 | − | 52396609 | 52397267 | 52396609 |
| 802 | MYO1G | chr7 | − | 44972114 | 44975227 | 44972114 |
| 3203 | PHYHD1 | chr9 | + | 128941444 | 128942038 | 128941444 |
| 350 | SNHG17 | chr20 | − | 38421005 | 38422241 | 38421005 |
| 1639 | TCL6 | chr14 | + | 95668125 | 95669617 | 95668125 |
| 2868 | NMRK1 | chr9 | − | 75068995 | 75070042 | 75068995 |

| | | | | | | IncLev- |
|---|---|---|---|---|---|---|
| | | | | | | el |
| | upstream | downstream | downstream | | | Differ- |
| ID | EE | ES | EE | PValue | FDR | ence |
| 2112 | 156596248 | 156596386 | 156596478 | 1.903E−03 | 1.964E−02 | 0.193 |
| 6377 | 42409995 | 42410431 | 42410496 | 4.781E−03 | 3.931E−02 | 0.177 |
| 4280 | 852667 | 852812 | 852952 | 8.122E−05 | 1.640E−03 | 0.174 |
| 2854 | 144829090 | 144829444 | 144829604 | 3.150E−03 | 2.951E−02 | 0.166 |
| 956 | 30645974 | 30646066 | 30646109 | 5.966E−05 | 1.297E−03 | 0.163 |
| 6880 | 8147002 | 8147249 | 8147381 | 1.403E−03 | 1.570E−02 | 0.162 |
| 991 | 155611355 | 155611548 | 155611603 | 1.029E−04 | 1.959E−03 | 0.158 |
| 4675 | 24000933 | 24001032 | 24001084 | 2.238E−04 | 3.661E−03 | 0.158 |
| 3464 | 65663683 | 65665420 | 65665543 | 2.382E−03 | 2.353E−02 | 0.154 |
| 5238 | 65112865 | 65113219 | 65113414 | 3.924E−06 | 1.296E−04 | 0.154 |
| 2533 | 23836962 | 23837063 | 23837154 | 1.111E−03 | 1.307E−02 | 0.148 |
| 3692 | 44181741 | 44182929 | 44183093 | 5.489E−03 | 4.383E−02 | 0.148 |
| 6333 | 30925939 | 30928552 | 30928656 | 2.484E−03 | 2.434E−02 | 0.147 |
| 5791 | 71656563 | 71656884 | 71657301 | 3.738E−08 | 2.979E−06 | 0.139 |
| 1952 | 143839549 | 143839735 | 143839825 | 3.680E−03 | 3.306E−02 | 0.137 |
| 6231 | 122975867 | 122976600 | 122976767 | 6.818E−05 | 1.420E−03 | 0.137 |
| 4279 | 852667 | 852809 | 852952 | 2.270E−05 | 5.654E−04 | 0.133 |
| 5676 | 53315140 | 53315726 | 53315782 | 4.042E−03 | 3.497E−02 | 0.127 |
| 5999 | 54594278 | 54594440 | 54594476 | 3.353E−03 | 3.103E−02 | 0.127 |
| 4158 | 3138179 | 3140385 | 3140509 | 9.516E−05 | 1.853E−03 | 0.125 |
| 5920 | 132141020 | 132141274 | 132141420 | 6.794E−08 | 4.778E−06 | 0.123 |
| 7297 | 9418895 | 9419050 | 9419299 | 1.607E−09 | 2.287E−07 | 0.118 |
| 2287 | 74423385 | 74423854 | 74423982 | 5.759E−08 | 4.303E−06 | 0.117 |
| 6335 | 218662260 | 218662514 | 218662679 | 2.939E−03 | 2.793E−02 | 0.116 |
| 5956 | 70466954 | 70467373 | 70467471 | 5.732E−05 | 1.260E−03 | 0.115 |
| 814 | 100505495 | 100506039 | 100506201 | 1.220E−05 | 3.456E−04 | 0.114 |
| 4875 | 89318037 | 89318540 | 89318749 | 2.157E−05 | 5.451E−04 | 0.114 |
| 505 | 118312170 | 118313706 | 118313874 | 6.253E−09 | 7.173E−07 | 0.113 |
| 3884 | 28932612 | 28932910 | 28933114 | 1.088E−03 | 1.283E−02 | 0.113 |
| 6388 | 43401850 | 43403682 | 43403799 | 5.760E−03 | 4.549E−02 | 0.112 |
| 5776 | 57807489 | 57807589 | 57807727 | 2.184E−04 | 3.596E−03 | 0.111 |
| 451 | 53091529 | 53091636 | 53091720 | 3.001E−03 | 2.834E−02 | 0.108 |
| 6105 | 4889763 | 4890221 | 4890735 | 2.043E−03 | 2.078E−02 | 0.108 |
| 1075 | 127724364 | 127724755 | 127724897 | 5.310E−03 | 4.271E−02 | 0.105 |
| 5044 | 6652770 | 6652944 | 6653050 | 1.088E−04 | 2.045E−03 | 0.105 |
| 2645 | 161674073 | 161677327 | 161677365 | 3.971E−05 | 9.166E−04 | 0.102 |
| 512 | 41541899 | 41541990 | 41545030 | 4.998E−04 | 6.775E−03 | 0.098 |
| 1558 | 7091680 | 7092386 | 7092441 | 1.808E−12 | 9.007E−10 | 0.097 |
| 6474 | 28904665 | 28908807 | 28908840 | 1.439E−06 | 5.659E−05 | 0.097 |
| 5134 | 125200577 | 125200779 | 125200906 | 2.024E−03 | 2.067E−02 | 0.096 |
| 6881 | 8229206 | 8229301 | 8229446 | 2.679E−05 | 6.591E−04 | 0.093 |
| 6467 | 75359153 | 75359327 | 75359425 | 3.497E−04 | 5.099E−03 | 0.091 |
| 4769 | 38420013 | 38420130 | 38420262 | 2.795E−03 | 2.686E−02 | 0.09 |
| 6555 | 685990 | 686067 | 686186 | 5.370E−03 | 4.299E−02 | 0.088 |
| 7458 | 38876490 | 38876692 | 38876781 | 3.329E−03 | 3.085E−02 | 0.087 |
| 1877 | 99619993 | 99621372 | 99621574 | 1.512E−03 | 1.661E−02 | 0.086 |
| 6230 | 122975867 | 122976583 | 122976767 | 1.946E−06 | 7.137E−05 | 0.086 |
| 2817 | 3891057 | 3892882 | 3892996 | 2.763E−03 | 2.668E−02 | 0.085 |
| 4726 | 136992700 | 136992924 | 136992974 | 3.216E−03 | 2.999E−02 | 0.081 |
| 2714 | 852110 | 852670 | 852766 | 1.720E−06 | 6.507E−05 | 0.08 |
| 98 | 49178179 | 49178271 | 49178475 | 1.969E−04 | 3.316E−03 | 0.078 |
| 3273 | 49664846 | 49665630 | 49665857 | 3.275E−06 | 1.138E−04 | 0.078 |
| 1466 | 45332834 | 45332917 | 45333324 | 2.150E−10 | 4.943E−08 | 0.077 |
| 1387 | 95410586 | 95410863 | 95411026 | 1.550E−05 | 4.251E−04 | 0.075 |
| 4336 | 7402715 | 7402816 | 7402897 | 1.648E−03 | 1.776E−02 | 0.074 |
| 6191 | 88609403 | 88609726 | 88609803 | 2.454E−04 | 3.881E−03 | 0.074 |
| 6738 | 67228836 | 67228975 | 67229278 | 7.492E−10 | 1.179E−07 | 0.074 |
| 3805 | 140693508 | 140693590 | 140693665 | 6.428E−07 | 3.026E−05 | 0.073 |
| 5227 | 64792086 | 64792171 | 64792422 | 1.064E−03 | 1.267E−02 | 0.072 |
| 3990 | 49014116 | 49014209 | 49014293 | 6.022E−04 | 7.948E−03 | 0.071 |
| 454 | 51899912 | 51900092 | 51900294 | 2.340E−08 | 2.057E−06 | 0.07 |
| 1669 | 113012811 | 113013271 | 113013403 | 5.383E−07 | 2.595E−05 | 0.07 |
| 5177 | 61960043 | 61962254 | 61962893 | 4.532E−06 | 1.459E−04 | 0.07 |
| 5309 | 3025988 | 3026250 | 3026288 | 0.000E+00 | 0.000E+00 | 0.069 |
| 5633 | 48135038 | 48135290 | 48135383 | 7.120E−04 | 9.153E−03 | 0.069 |
| 345 | 44622930 | 44623006 | 44623078 | 4.543E−05 | 1.036E−03 | 0.068 |
| 6166 | 76082367 | 76082616 | 76082692 | 1.971E−05 | 5.080E−04 | 0.067 |
| 3341 | 39742031 | 39743215 | 39743297 | 2.880E−03 | 2.746E−02 | 0.065 |
| 1480 | 26444304 | 26445703 | 26445985 | 1.846E−03 | 1.926E−02 | 0.064 |
| 650 | 93145532 | 93145908 | 93146040 | 5.564E−03 | 4.423E−02 | 0.062 |
| 7170 | 11886621 | 11886712 | 11886778 | 2.103E−03 | 2.125E−02 | 0.062 |
| 4667 | 6548157 | 6548424 | 6548545 | 3.453E−03 | 3.171E−02 | 0.061 |
| 1759 | 71068712 | 71070501 | 71070586 | 2.048E−03 | 2.079E−02 | 0.06 |
| 7315 | 11353572 | 11353791 | 11353874 | 8.544E−04 | 1.055E−02 | 0.06 |
| 3316 | 75705693 | 75706007 | 75706092 | 1.263E−03 | 1.452E−02 | 0.059 |
| 3511 | 179837885 | 179840871 | 179840959 | 3.321E−03 | 3.083E−02 | 0.059 |
| 6087 | 57476651 | 57476870 | 57476963 | 1.261E−07 | 8.022E−06 | 0.059 |
| 1342 | 64425138 | 64425337 | 64425457 | 8.289E−04 | 1.034E−02 | 0.058 |
| 2324 | 42746852 | 42747271 | 42747412 | 1.263E−04 | 2.310E−03 | 0.058 |
| 6079 | 24173041 | 24173125 | 24173198 | 2.846E−04 | 4.408E−03 | 0.057 |
| 2196 | 107745143 | 107746688 | 107746889 | 1.762E−04 | 3.018E−03 | 0.056 |
| 6015 | 20455328 | 20455577 | 20455703 | 2.370E−04 | 3.818E−03 | 0.056 |
| 7124 | 80415518 | 80415649 | 80415821 | 3.661E−03 | 3.306E−02 | 0.056 |
| 1943 | 119092163 | 119092403 | 119092523 | 5.259E−05 | 1.173E−03 | 0.055 |
| 3408 | 9745709 | 9745811 | 9745930 | 1.571E−03 | 1.710E−02 | 0.055 |
| 4440 | 24292330 | 24292651 | 24292784 | 2.941E−05 | 7.146E−04 | 0.054 |
| 4554 | 132684794 | 132686259 | 132686387 | 1.280E−03 | 1.461E−02 | 0.054 |
| 4628 | 107487192 | 107487294 | 107488267 | 7.516E−14 | 6.419E−11 | 0.054 |
| 5685 | 53713900 | 53714132 | 53714241 | 1.805E−03 | 1.893E−02 | 0.054 |
| 6355 | 5426299 | 5428070 | 5428211 | 3.432E−07 | 1.789E−05 | 0.054 |
| 1718 | 1272051 | 1274306 | 1274440 | 9.286E−04 | 1.131E−02 | 0.053 |
| 2447 | 92235646 | 92236412 | 92236542 | 3.168E−03 | 2.964E−02 | 0.053 |
| 3163 | 32394140 | 32394403 | 32394425 | 9.982E−05 | 1.925E−03 | 0.053 |
| 4469 | 34648456 | 34648761 | 34648894 | 7.107E−04 | 9.153E−03 | 0.053 |
| 6222 | 85601107 | 85601434 | 85601482 | 1.776E−03 | 1.869E−02 | 0.053 |
| 1130 | 73794330 | 73794555 | 73794639 | 2.201E−03 | 2.212E−02 | 0.052 |
| 1409 | 2040374 | 2040504 | 2040605 | 4.902E−03 | 3.998E−02 | 0.052 |
| 7169 | 11345628 | 11346207 | 11346268 | 1.226E−05 | 3.456E−04 | 0.052 |
| 1349 | 48232493 | 48234402 | 48234543 | 6.938E−04 | 8.977E−03 | 0.051 |
| 1569 | 2525318 | 2525413 | 2525567 | 5.963E−08 | 4.347E−06 | 0.051 |
| 3080 | 37241637 | 37242634 | 37242693 | 3.261E−04 | 4.849E−03 | 0.051 |
| 3717 | 106557131 | 106559431 | 106559657 | 4.992E−06 | 1.579E−04 | 0.051 |
| 1050 | 176529818 | 176530081 | 176530206 | 3.424E−05 | 8.123E−04 | 0.05 |
| 1951 | 143835875 | 143835966 | 143836030 | 1.419E−06 | 5.656E−05 | 0.05 |
| 2751 | 2669561 | 2671594 | 2672570 | 2.295E−06 | 8.315E−05 | 0.05 |
| 2853 | 144829090 | 144829426 | 144829604 | 1.138E−07 | 7.473E−06 | −0.05 |
| 990 | 155611291 | 155611548 | 155611603 | 4.868E−05 | 1.106E−03 | −0.052 |
| 2218 | 1628399 | 1628488 | 1628722 | 1.270E−05 | 3.564E−04 | −0.055 |
| 6038 | 35546808 | 35547118 | 35547240 | 1.275E−04 | 2.324E−03 | −0.055 |
| 5988 | 66515586 | 66515692 | 66515731 | 3.365E−04 | 4.942E−03 | −0.057 |
| 450 | 53091497 | 53091636 | 53091720 | 4.296E−11 | 1.110E−08 | −0.058 |
| 455 | 51886912 | 51887831 | 51887936 | 2.029E−05 | 5.205E−04 | −0.059 |
| 2270 | 39967711 | 39967803 | 39967916 | 3.401E−04 | 4.983E−03 | −0.064 |
| 5428 | 119078334 | 119078794 | 119078997 | 2.556E−08 | 2.214E−06 | −0.064 |
| 5051 | 161170949 | 161171033 | 161171181 | 6.327E−03 | 4.925E−02 | −0.065 |
| 5810 | 47226027 | 47226582 | 47226703 | 3.466E−04 | 5.066E−03 | −0.069 |
| 2215 | 1625653 | 1626649 | 1626754 | 4.909E−03 | 3.998E−02 | −0.074 |
| 5327 | 49642372 | 49642579 | 49642675 | 1.825E−03 | 1.909E−02 | −0.079 |
| 1174 | 81322540 | 81323801 | 81324141 | 1.831E−06 | 6.840E−05 | −0.083 |
| 2216 | 1626999 | 1627073 | 1627207 | 1.475E−07 | 9.092E−06 | −0.085 |
| 5811 | 47227099 | 47227159 | 47227223 | 1.357E−03 | 1.522E−02 | −0.085 |
| 5409 | 111786094 | 111786401 | 111786520 | 7.343E−05 | 1.508E−03 | −0.089 |
| 4758 | 39547634 | 39548861 | 39548951 | 3.085E−04 | 4.670E−03 | −0.099 |
| 1291 | 74531411 | 74531596 | 74531702 | 2.829E−03 | 2.710E−02 | −0.113 |
| 6466 | 75358618 | 75358703 | 75358808 | 2.376E−03 | 2.353E−02 | −0.124 |
| 453 | 52396752 | 52397194 | 52397267 | 1.361E−05 | 3.801E−04 | −0.131 |
| 802 | 44972274 | 44975173 | 44975227 | 1.665E−03 | 1.781E−02 | −0.16 |
| 3203 | 128941571 | 128941667 | 128942038 | 1.280E−03 | 1.461E−02 | −0.162 |
| 350 | 38421098 | 38422091 | 38422241 | 6.789E−05 | 1.419E−03 | −0.165 |
| 1639 | 95668580 | 95669550 | 95669617 | 2.476E−05 | 6.141E−04 | −0.167 |
| 2868 | 75069102 | 75069894 | 75070042 | 3.938E−10 | 6.897E−08 | −0.264 |

TABLE 20
CARRIER_VS_TD_SE

| | | | exon | exon | upstream | upstream | downstream |
|---|---|---|---|---|---|---|---|
| geneSymbol | chr | strand | Start_0base | End | ES | EE | ES |
| EXOC7 | chr17 | − | 76091142 | 76091235 | 76089174 | 76089320 | 76094413 |
| LRRC23 | chr12 | + | 6909889 | 6910026 | 6907314 | 6907445 | 6913890 |
| TPD52L1 | chr6 | + | 125252021 | 125252036 | 125248281 | 125248383 | 125253716 |
| NRBP1 | chr2 | + | 27435722 | 27435746 | 27435132 | 27435227 | 27436752 |
| COX5A | chr15 | − | 74926765 | 74926887 | 74923593 | 74923770 | 74929115 |
| UBE2Q2 | chr15 | + | 75859877 | 75859982 | 75854385 | 75854487 | 75868950 |
| SAT2 | chr17 | − | 7626942 | 7627044 | 7626752 | 7626793 | 7627142 |
| ABLIM1 | chr10 | − | 114447879 | 114448026 | 114445311 | 114445403 | 114451623 |
| TTC3 | chr21 | + | 37150819 | 37150884 | 37150077 | 37150170 | 37151892 |
| SZRD1 | chr1 | + | 16391374 | 16391424 | 16367082 | 16367308 | 16393230 |
| KIF21A | chr12 | − | 39346465 | 39346504 | 39342033 | 39342124 | 39351776 |
| CDC16 | chr13 | + | 114261886 | 114261948 | 114259334 | 114259398 | 114262878 |
| PRPF38B | chr1 | + | 108693589 | 108693642 | 108692340 | 108692867 | 108695701 |
| MOBP | chr3 | + | 39499466 | 39499532 | 39480039 | 39480123 | 39502065 |
| ZNF148 | chr3 | − | 125314936 | 125315053 | 125313307 | 125313656 | 125323308 |
| GAB1 | chr4 | + | 143457679 | 143457769 | 143440078 | 143440382 | 143459384 |
| PCDHGC3 | chr5 | + | 141478393 | 141478546 | 141476000 | 141476146 | 141494806 |
| ZFAND5 | chr9 | − | 72363469 | 72363606 | 72360627 | 72360787 | 72364695 |
| SGIP1 | chr1 | + | 66695433 | 66695493 | 66690189 | 66690316 | 66719293 |
| LRRC75A-AS1 | chr17 | + | 16439527 | 16439703 | 16439062 | 16439414 | 16440184 |
| LRRC75A-AS1 | chr17 | + | 16439580 | 16439703 | 16439326 | 16439414 | 16440184 |
| LRRC75A-AS1 | chr17 | + | 16439659 | 16439703 | 16439044 | 16439414 | 16440184 |
| PCBP4 | chr3 | − | 51962809 | 51962892 | 51961159 | 51961304 | 51967325 |
| CAMK2A | chr5 | − | 150239703 | 150239736 | 150238699 | 150238748 | 150245160 |
| CWC27 | chr5 | + | 64977134 | 64977238 | 64971702 | 64971812 | 65018158 |
| ARPP21 | chr3 | + | 35717297 | 35717357 | 35715438 | 35715476 | 35721604 |
| LSM14A | chr19 | + | 34226407 | 34226464 | 34221506 | 34221738 | 34227364 |
| PCBP2 | chr12 | + | 53467804 | 53467843 | 53467220 | 53467293 | 53468779 |
| ARHGAP26 | chr5 | + | 143207197 | 143207473 | 143147230 | 143147381 | 143213996 |
| IDH3B | chr20 | − | 2658758 | 2658837 | 2658394 | 2658522 | 2659524 |
| NOVA1 | chr14 | − | 26472319 | 26472391 | 26445888 | 26448963 | 26479976 |
| LINC-PINT | chr7 | − | 130959868 | 130959970 | 130945719 | 130945835 | 130984052 |
| ZNF207 | chr17 | + | 32366664 | 32366757 | 32365329 | 32365487 | 32367771 |
| GKAP1 | chr9 | − | 83768817 | 83768970 | 83753257 | 83753359 | 83780381 |
| CDC42BPA | chr1 | − | 227112670 | 227112913 | 227112311 | 227112422 | 227119803 |
| MAP7D1 | chr1 | + | 36174897 | 36175008 | 36173363 | 36173478 | 36176198 |
| PAQR6 | chr1 | − | 156245534 | 156245661 | 156245141 | 156245238 | 156245781 |
| MADD | chr11 | + | 47288967 | 47289027 | 47286432 | 47286534 | 47289390 |
| MADD | chr11 | + | 47308979 | 47309042 | 47308590 | 47308699 | 47309280 |
| KIF13A | chr6 | − | 17789871 | 17789910 | 17787775 | 17787875 | 17794248 |
| UNC79 | chr14 | + | 93634520 | 93634637 | 93630800 | 93630908 | 93637215 |
| NCAM1 | chr11 | + | 113236300 | 113236315 | 113235032 | 113235164 | 113255876 |
| NCAM1 | chr11 | + | 113246367 | 113246370 | 113235032 | 113235164 | 113255876 |
| CAMK2D | chr4 | − | 113502935 | 113502977 | 113500462 | 113500511 | 113509637 |
| TRIM33 | chr1 | − | 114397939 | 114397990 | 114392776 | 114397860 | 114399456 |
| CCAR1 | chr10 | + | 68756272 | 68756483 | 68755369 | 68755536 | 68757293 |
| RP5-1042K10.14 | chr22 | + | 40364879 | 40365056 | 40364275 | 40364365 | 40366435 |
| CAPN3 | chr15 | + | 42408210 | 42408324 | 42404779 | 42404955 | 42409302 |
| HSD11B1L | chr19 | + | 5686415 | 5686527 | 5684964 | 5685119 | 5686899 |
| UQCRH | chr1 | + | 46309100 | 46309127 | 46303630 | 46303820 | 46310154 |
| MARK2 | chr11 | + | 63908259 | 63908304 | 63904785 | 63905043 | 63908876 |
| BIN1 | chr2 | − | 127051153 | 127051243 | 127050801 | 127050912 | 127059010 |
| AIFM3 | chr22 | + | 20980746 | 20980767 | 20980019 | 20980124 | 20980991 |
| COG1 | chr17 | + | 73207701 | 73207739 | 73207180 | 73207256 | 73208313 |
| SORBS1 | chr10 | − | 95414493 | 95414862 | 95410632 | 95410777 | 95421960 |
| BAZ2B | chr2 | − | 159400598 | 159400664 | 159398828 | 159398894 | 159404848 |
| BRSK2 | chr11 | + | 1459191 | 1459239 | 1456597 | 1456687 | 1460969 |
| ARMC10 | chr7 | + | 103092476 | 103092653 | 103086629 | 103086764 | 103097276 |
| AC005154.6 | chr7 | − | 30552099 | 30552179 | 30550635 | 30550781 | 30561465 |
| LCMT1 | chr16 | + | 25161101 | 25161204 | 25140170 | 25140247 | 25164597 |
| USMG5 | chr10 | − | 103394198 | 103394394 | 103392370 | 103392466 | 103395745 |
| USMG5 | chr10 | − | 103394198 | 103394394 | 103392370 | 103392466 | 103396408 |
| CLSTN1 | chr1 | − | 9737497 | 9737554 | 9735884 | 9736042 | 9741093 |
| ZMIZ2 | chr7 | + | 44760150 | 44760228 | 44759280 | 44759460 | 44760424 |
| UQCRB | chr8 | − | 96231378 | 96231515 | 96230941 | 96231132 | 96231773 |
| SH3GLB2 | chr9 | − | 129021090 | 129021219 | 129014770 | 129014904 | 129028091 |
| CAMK2G | chr10 | − | 73825278 | 73825347 | 73824039 | 73824084 | 73828088 |
| CAMK2G | chr10 | − | 73828088 | 73828121 | 73824039 | 73824084 | 73837467 |
| CAMK2G | chr10 | − | 73828088 | 73828121 | 73825278 | 73825347 | 73837467 |
| ACBD4 | chr17 | + | 45137018 | 45137139 | 45136691 | 45136776 | 45137367 |
| GPM6B | chrX | − | 13807649 | 13807769 | 13785621 | 13785808 | 13938506 |
| MTDH | chr8 | + | 97699753 | 97699852 | 97690951 | 97691188 | 97706625 |
| MTDH | chr8 | + | 97719048 | 97719189 | 97713661 | 97713769 | 97722878 |
| MAP4K4 | chr2 | + | 101860824 | 101860986 | 101859642 | 101859864 | 101863820 |
| MAP4K4 | chr2 | + | 101863820 | 101864051 | 101860824 | 101860986 | 101864929 |
| BCAS1 | chr20 | − | 53957431 | 53957497 | 53953431 | 53953695 | 53966905 |
| TRAPPC12 | chr2 | + | 3477694 | 3477795 | 3465596 | 3465695 | 3478845 |
| PHYHIP | chr8 | − | 22228787 | 22228909 | 22228192 | 22228386 | 22231795 |
| SYNE1 | chr6 | − | 152130719 | 152130778 | 152121683 | 152122676 | 152132121 |
| KCNC3 | chr19 | − | 50316032 | 50316091 | 50311936 | 50314990 | 50320592 |
| ACAA1 | chr3 | − | 38126161 | 38126341 | 38125825 | 38125881 | 38126509 |
| RNF146 | chr6 | + | 127285207 | 127285348 | 127280230 | 127280340 | 127286049 |
| GSTO1 | chr10 | + | 104262978 | 104263077 | 104259575 | 104259798 | 104266083 |
| ARHGAP12 | chr10 | − | 31839636 | 31839711 | 31831738 | 31831800 | 31843460 |
| AP1G2 | chr14 | − | 23565815 | 23565892 | 23565605 | 23565701 | 23566063 |
| PHLDB1 | chr11 | + | 118655859 | 118655892 | 118655604 | 118655690 | 118656682 |
| SH3YL1 | chr2 | − | 224863 | 224920 | 218148 | 219001 | 229965 |
| NRCAM | chr7 | − | 108237751 | 108237769 | 108234582 | 108234688 | 108239958 |
| ASPH | chr8 | − | 61646749 | 61646878 | 61644599 | 61644632 | 61651049 |
| ASPDH | chr19 | − | 50512926 | 50513011 | 50512135 | 50512290 | 50514477 |
| ZIM2 | chr19 | − | 56826387 | 56826463 | 56824261 | 56824427 | 56836017 |
| CHL1 | chr3 | + | 342983 | 343031 | 341911 | 342082 | 344588 |
| COL16A1 | chr1 | − | 31679803 | 31679851 | 31679631 | 31679685 | 31680041 |
| PRKACB | chr1 | + | 84175798 | 84175807 | 84175032 | 84175056 | 84179176 |
| GANAB | chr11 | − | 62634309 | 62634375 | 62633444 | 62633514 | 62634820 |
| RHBDD2 | chr7 | + | 75881364 | 75881486 | 75878998 | 75879260 | 75881828 |
| TJP1 | chr15 | − | 29719776 | 29720016 | 29718265 | 29719138 | 29720357 |
| KIF3A | chr5 | − | 132708906 | 132708974 | 132706450 | 132706459 | 132710958 |
| GOLIM4 | chr3 | − | 168040785 | 168040869 | 168036835 | 168036994 | 168041391 |
| CBWD1 | chr9 | − | 177722 | 177820 | 175697 | 175784 | 178815 |
| ITSN2 | chr2 | − | 24284762 | 24284843 | 24275712 | 24275849 | 24286211 |
| DAP3 | chr1 | + | 155729041 | 155729122 | 155727607 | 155727738 | 155729207 |
| CCDC136 | chr7 | + | 128806236 | 128806395 | 128805760 | 128805901 | 128817757 |
| CLASP2 | chr3 | − | 33577201 | 33577264 | 33576168 | 33576275 | 33581820 |
| NCOA1 | chr2 | + | 24768078 | 24768135 | 24762686 | 24762776 | 24768223 |
| GNAS | chr20 | + | 58898940 | 58898985 | 58895611 | 58895684 | 58903530 |
| RBFOX1 | chr16 | + | 7693314 | 7693367 | 7676773 | 7676838 | 7709055 |
| CEP290 | chr12 | − | 88054339 | 88054413 | 88053651 | 88053746 | 88055575 |
| PUF60 | chr8 | − | 143820665 | 143820716 | 143818372 | 143818534 | 143821596 |
| SLC25A23 | chr19 | − | 6453980 | 6454088 | 6452311 | 6452479 | 6454322 |
| MEG3 | chr14 | + | 100834631 | 100834751 | 100831420 | 100831534 | 100835738 |
| MEG3 | chr14 | + | 100835738 | 100835879 | 100829033 | 100831534 | 100836166 |
| FN1 | chr2 | − | 215372108 | 215372300 | 215371908 | 215372015 | 215373321 |
| FN1 | chr2 | − | 215372108 | 215372375 | 215371908 | 215372015 | 215373321 |
| DTNA | chr18 | + | 34866110 | 34866131 | 34863965 | 34864062 | 34875238 |
| GPM6A | chr4 | − | 175812190 | 175812249 | 175701574 | 175701767 | 176002308 |
| OLA1 | chr2 | − | 174246714 | 174246815 | 174229307 | 174229451 | 174248451 |
| YWHAZ | chr8 | − | 100951928 | 100952125 | 100948595 | 100948900 | 100952849 |
| RNASE1 | chr14 | − | 20802248 | 20802319 | 20801345 | 20802093 | 20802796 |
| RNASE1 | chr14 | − | 20802248 | 20802331 | 20801459 | 20802093 | 20802796 |
| CALU | chr7 | + | 128754528 | 128754722 | 128754261 | 128754455 | 128758870 |
| ALG8 | chr11 | − | 78102873 | 78102948 | 78100946 | 78101195 | 78103979 |

| | downstream | | | | IncLevel | |
|---|---|---|---|---|---|---|
| geneSymbol | EE | ID. 1 | PValue | FDR | Difference | type |
| EXOC7 | 76094581 | 57 | 9.336E−09 | 8.572E−06 | 0.302 | UP |
| LRRC23 | 6914228 | 578 | 3.214E−04 | 1.395E−02 | −0.233 | DN |
| TPD52L1 | 125253755 | 736 | 5.233E−05 | 4.215E−03 | 0.164 | UP |
| NRBP1 | 27436836 | 893 | 6.485E−04 | 2.085E−02 | −0.141 | DN |
| COX5A | 74929232 | 1316 | 9.618E−04 | 2.686E−02 | −0.018 | NC |
| UBE2Q2 | 75869010 | 2080 | 5.243E−04 | 1.843E−02 | 0.204 | UP |
| SAT2 | 7627226 | 2121 | 1.382E−09 | 2.256E−06 | −0.323 | DN |
| ABLIM1 | 114451671 | 2315 | 1.183E−03 | 3.071E−02 | −0.111 | DN |
| TTC3 | 37152029 | 3069 | 8.095E−04 | 2.390E−02 | 0.015 | NC |
| SZRD1 | 16393482 | 3258 | 1.393E−04 | 8.369E−03 | 0.421 | UP |
| KIF21A | 39351980 | 3325 | 5.015E−04 | 1.793E−02 | 0.084 | UP |
| CDC16 | 114263014 | 3590 | 6.930E−04 | 2.168E−02 | 0.121 | UP |
| PRPF38B | 108695770 | 3866 | 2.244E−03 | 4.604E−02 | 0.139 | UP |
| MOBP | 39502275 | 4038 | 1.159E−03 | 3.027E−02 | 0.055 | UP |
| ZNF148 | 125323444 | 4438 | 1.378E−03 | 3.374E−02 | 0.099 | UP |
| GAB1 | 143459478 | 4490 | 6.484E−04 | 2.085E−02 | −0.354 | DN |
| PCDHGC3 | 141494865 | 5145 | 2.255E−04 | 1.127E−02 | −0.186 | DN |
| ZFAND5 | 72365197 | 5416 | 4.210E−04 | 1.619E−02 | 0.125 | UP |
| SGIP1 | 66719405 | 6351 | 9.824E−04 | 2.726E−02 | 0.25 | UP |
| LRRC75A-AS1 | 16440253 | 6368 | 8.350E−09 | 7.915E−06 | −0.061 | DN |
| LRRC75A-AS1 | 16440253 | 6369 | 9.845E−10 | 1.808E−06 | −0.065 | DN |
| LRRC75A-AS1 | 16440253 | 6370 | 6.767E−07 | 1.930E−04 | −0.038 | NC |
| PCBP4 | 51967434 | 6759 | 1.873E−07 | 8.092E−05 | 0.257 | UP |
| CAMK2A | 150245201 | 6984 | 8.023E−05 | 5.573E−03 | −0.091 | DN |
| CWC27 | 65018763 | 7056 | 1.918E−03 | 4.167E−02 | −0.084 | DN |
| ARPP21 | 35721834 | 7468 | 4.579E−06 | 8.794E−04 | 0.257 | UP |
| LSM14A | 34228096 | 7572 | 2.334E−05 | 2.554E−03 | 0.232 | UP |
| PCBP2 | 53468832 | 8344 | 3.383E−04 | 1.426E−02 | 0.149 | UP |
| ARHGAP26 | 143214088 | 8395 | 2.460E−03 | 4.886E−02 | −0.175 | DN |
| IDH3B | 2659585 | 8707 | 5.671E−04 | 1.933E−02 | 0.124 | UP |
| NOVA1 | 26480143 | 8812 | 1.746E−03 | 3.928E−02 | −0.12 | DN |
| LINC-PINT | 130984109 | 9070 | 1.306E−03 | 3.271E−02 | 0.081 | UP |
| ZNF207 | 32368014 | 9312 | 1.219E−06 | 3.000E−04 | −0.229 | DN |
| GKAP1 | 83780404 | 9545 | 2.023E−03 | 4.311E−02 | −0.152 | DN |
| CDC42BPA | 227119937 | 9802 | 7.206E−04 | 2.217E−02 | 0.068 | UP |
| MAP7D1 | 36176321 | 10042 | 4.227E−04 | 1.622E−02 | −0.069 | DN |
| PAQR6 | 156245987 | 10889 | 3.719E−04 | 1.524E−02 | 0.093 | UP |
| MADD | 47289493 | 11015 | 1.551E−03 | 3.635E−02 | −0.233 | DN |
| MADD | 47309401 | 11017 | 1.435E−03 | 3.454E−02 | 0.122 | UP |
| KIF13A | 17794395 | 11159 | 1.473E−04 | 8.652E−03 | 0.293 | UP |
| UNC79 | 93637299 | 11543 | 3.592E−04 | 1.484E−02 | −0.457 | DN |
| NCAM1 | 113256001 | 11574 | 2.966E−05 | 2.935E−03 | −0.179 | DN |
| NCAM1 | 113256001 | 11578 | 1.197E−03 | 3.080E−02 | −0.155 | DN |
| CAMK2D | 113509675 | 11754 | 1.784E−03 | 3.973E−02 | −0.362 | DN |
| TRIM33 | 114399609 | 11810 | 2.152E−03 | 4.488E−02 | 0.117 | UP |
| CCAR1 | 68757377 | 11969 | 8.100E−05 | 5.613E−03 | −0.266 | DN |
| RP5-1042K10.14 | 40366498 | 12020 | 1.981E−03 | 4.249E−02 | −0.113 | DN |
| CAPN3 | 42409380 | 12237 | 2.264E−04 | 1.128E−02 | 0.284 | UP |
| HSD11B1L | 5686991 | 12288 | 1.799E−03 | 3.992E−02 | −0.185 | DN |
| UQCRH | 46310316 | 12602 | 2.300E−11 | 2.253E−07 | 0.058 | UP |
| MARK2 | 63909237 | 12666 | 1.404E−04 | 8.402E−03 | 0.234 | UP |
| BIN1 | 127059155 | 12797 | 1.491E−03 | 3.550E−02 | 0.043 | NC |
| AIFM3 | 20981360 | 12817 | 3.665E−06 | 7.748E−04 | −0.104 | DN |
| COG1 | 73208503 | 13141 | 2.817E−07 | 1.104E−04 | 0.166 | UP |
| SORBS1 | 95422073 | 13810 | 1.806E−03 | 4.003E−02 | −0.075 | DN |
| BAZ2B | 159404901 | 13893 | 1.091E−03 | 2.903E−02 | 0.046 | NC |
| BRSK2 | 1462087 | 14919 | 5.302E−04 | 1.852E−02 | −0.092 | DN |
| ARMC10 | 103097348 | 15090 | 3.902E−04 | 1.563E−02 | −0.259 | DN |
| AC005154.6 | 30561516 | 15347 | 5.762E−04 | 1.943E−02 | 0.182 | UP |
| LCMT1 | 25164718 | 15589 | 1.330E−04 | 8.077E−03 | 0.114 | UP |
| USMG5 | 103396023 | 15747 | 2.731E−06 | 5.988E−04 | 0.187 | UP |
| USMG5 | 103396466 | 15748 | 1.353E−08 | 1.046E−05 | 0.032 | NC |
| CLSTN1 | 9741256 | 15924 | 9.392E−04 | 2.647E−02 | 0.027 | NC |
| ZMIZ2 | 44760593 | 16216 | 2.838E−04 | 1.305E−02 | 0.118 | UP |
| UQCRB | 96231940 | 16218 | 4.329E−08 | 2.596E−05 | 0.014 | NC |
| SH3GLB2 | 129028185 | 16619 | 2.424E−03 | 4.833E−02 | −0.082 | DN |
| CAMK2G | 73828121 | 16812 | 1.997E−03 | 4.267E−02 | 0.203 | UP |
| CAMK2G | 73837511 | 16815 | 6.702E−04 | 2.121E−02 | −0.143 | DN |
| CAMK2G | 73837511 | 16816 | 2.126E−03 | 4.445E−02 | −0.141 | DN |
| ACBD4 | 45137454 | 17631 | 6.963E−04 | 2.170E−02 | −0.181 | DN |
| GPM6B | 13938638 | 17724 | 9.764E−06 | 1.393E−03 | 0.141 | UP |
| MTDH | 97706750 | 18203 | 2.827E−04 | 1.304E−02 | −0.139 | DN |
| MTDH | 97723035 | 18207 | 1.455E−05 | 1.823E−03 | 0.046 | NC |
| MAP4K4 | 101864051 | 18307 | 2.073E−03 | 4.372E−02 | 0.148 | UP |
| MAP4K4 | 101865036 | 18310 | 2.520E−10 | 6.730E−07 | −0.335 | DN |
| BCAS1 | 53967073 | 18456 | 1.462E−04 | 8.624E−03 | 0.079 | UP |
| TRAPPC12 | 3478933 | 18650 | 1.857E−03 | 4.088E−02 | 0.107 | UP |
| PHYHIP | 22232101 | 18740 | 1.611E−05 | 1.961E−03 | 0.122 | UP |
| SYNE1 | 152132214 | 19485 | 3.398E−04 | 1.429E−02 | 0.156 | UP |
| KCNC3 | 50320784 | 20995 | 1.119E−04 | 7.088E−03 | 0.186 | UP |
| ACAA1 | 38126700 | 21002 | 5.735E−04 | 1.939E−02 | −0.068 | DN |
| RNF146 | 127286177 | 21408 | 2.765E−04 | 1.288E−02 | 0.211 | UP |
| GSTO1 | 104266190 | 21411 | 2.591E−07 | 1.057E−04 | −0.081 | DN |
| ARHGAP12 | 31843586 | 21600 | 9.202E−04 | 2.613E−02 | 0.321 | UP |
| AP1G2 | 23566160 | 22217 | 4.263E−05 | 3.684E−03 | −0.062 | DN |
| PHLDB1 | 118658031 | 22721 | 9.446E−05 | 6.237E−03 | 0.184 | UP |
| SH3YL1 | 230044 | 23403 | 4.862E−04 | 1.764E−02 | 0.307 | UP |
| NRCAM | 108240049 | 23471 | 2.906E−04 | 1.324E−02 | 0.149 | UP |
| ASPH | 61651124 | 23768 | 1.042E−03 | 2.828E−02 | 0.122 | UP |
| ASPDH | 50514690 | 24160 | 1.083E−03 | 2.891E−02 | −0.271 | DN |
| ZIM2 | 56836078 | 24240 | 5.825E−04 | 1.956E−02 | 0.205 | UP |
| CHL1 | 344709 | 24529 | 6.118E−04 | 2.016E−02 | −0.155 | DN |
| COL16A1 | 31680101 | 24995 | 2.219E−03 | 4.572E−02 | −0.178 | DN |
| PRKACB | 84179238 | 25091 | 1.795E−05 | 2.093E−03 | 0.291 | UP |
| GANAB | 62635000 | 25238 | 2.506E−06 | 5.579E−04 | −0.204 | DN |
| RHBDD2 | 75882236 | 26294 | 1.307E−03 | 3.271E−02 | 0.077 | UP |
| TJP1 | 29720708 | 26778 | 3.717E−06 | 7.802E−04 | −0.252 | DN |
| KIF3A | 132711057 | 26789 | 9.442E−06 | 1.370E−03 | −0.075 | DN |
| GOLIM4 | 168041474 | 27443 | 1.176E−03 | 3.061E−02 | 0.18 | UP |
| CBWD1 | 179023 | 27770 | 4.218E−04 | 1.620E−02 | −0.408 | DN |
| ITSN2 | 24286351 | 28848 | 7.090E−06 | 1.157E−03 | 0.194 | UP |
| DAP3 | 155729366 | 28942 | 8.259E−04 | 2.427E−02 | 0.086 | UP |
| CCDC136 | 128817864 | 29566 | 1.655E−03 | 3.790E−02 | −0.083 | DN |
| CLASP2 | 33581928 | 29594 | 9.036E−04 | 2.588E−02 | −0.2 | DN |
| NCOA1 | 24768543 | 29661 | 2.159E−05 | 2.421E−03 | 0.222 | UP |
| GNAS | 58903585 | 30924 | 1.645E−03 | 3.776E−02 | 0.021 | NC |
| RBFOX1 | 7709131 | 31043 | 9.968E−04 | 2.737E−02 | 0.155 | UP |
| CEP290 | 88055717 | 31262 | 9.266E−05 | 6.185E−03 | 0.241 | UP |
| PUF60 | 143821686 | 32289 | 6.519E−05 | 4.892E−03 | 0.164 | UP |
| SLC25A23 | 6454475 | 32467 | 1.127E−03 | 2.964E−02 | −0.034 | NC |
| MEG3 | 100835879 | 32697 | 6.169E−05 | 4.764E−03 | −0.048 | NC |
| MEG3 | 100836300 | 32703 | 1.420E−03 | 3.435E−02 | 0.012 | NC |
| FN1 | 215373411 | 34160 | 3.986E−06 | 8.189E−04 | 0.16 | UP |
| FN1 | 215373411 | 34161 | 7.234E−07 | 2.024E−04 | 0.116 | UP |
| DTNA | 34875398 | 35553 | 2.288E−03 | 4.653E−02 | −0.082 | DN |
| GPM6A | 176002332 | 35558 | 1.239E−08 | 1.020E−05 | 0.074 | UP |
| OLA1 | 174248475 | 35825 | 5.701E−04 | 1.937E−02 | −0.155 | DN |
| YWHAZ | 100952983 | 35847 | 6.177E−05 | 4.764E−03 | 0.101 | UP |
| RNASE1 | 20802855 | 36230 | 9.313E−05 | 6.185E−03 | −0.172 | DN |
| RNASE1 | 20802848 | 36231 | 6.424E−05 | 4.875E−03 | −0.179 | DN |
| CALU | 128759037 | 37018 | 1.873E−03 | 4.110E−02 | 0.098 | UP |
| ALG8 | 78104052 | 37102 | 3.694E−07 | 1.292E−04 | 0.117 | UP |

TABLE 21
CARRIER_VS_TD_MXE

| | | | 1stExon | 1stExon | 2ndExon | | upstream |
|---|---|---|---|---|---|---|---|
| geneSymbol | chr | strand | Start_0base | End | Start_0base | 2ndExonEnd | ES |
| FYN | chr6 | − | 111699514 | 111699670 | 111700103 | 111700268 | 111696276 |
| PIBF1 | chr13 | + | 72965273 | 72965404 | 72973590 | 72973675 | 72931164 |
| TBC1D5 | chr3 | − | 17238162 | 17238419 | 17258505 | 17258591 | 17214206 |
| TNRC6A | chr16 | + | 24758338 | 24758360 | 24776932 | 24777358 | 24750725 |
| SEZ6L | chr22 | + | 26365371 | 26365566 | 26375574 | 26375689 | 26351051 |
| SEZ6L | chr22 | + | 26365371 | 26365566 | 26375577 | 26375689 | 26351051 |
| ZMAT4 | chr8 | − | 40581164 | 40581261 | 40674703 | 40674931 | 40530593 |
| MOBP | chr3 | + | 39480039 | 39480123 | 39499466 | 39499532 | 39467604 |
| PDXDC1 | chr16 | + | 15001775 | 15001856 | 15004186 | 15004333 | 14998339 |
| TPM3 | chr1 | − | 154170648 | 154170711 | 154172028 | 154172104 | 154170399 |
| ANKRD24 | chr19 | + | 4199874 | 4200005 | 4200082 | 4200171 | 4186269 |
| WIPF2 | chr17 | + | 40262524 | 40262641 | 40264489 | 40265146 | 40260534 |
| ATRNL1 | chr10 | + | 115315517 | 115315736 | 115334281 | 115334419 | 115301854 |
| FXYD7 | chr19 | + | 35151253 | 35151328 | 35151439 | 35151473 | 35148693 |
| TSSC1 | chr2 | − | 3257298 | 3257455 | 3338016 | 3338149 | 3214148 |
| RP11-21J18.1 | chr18 | + | 9124873 | 9124983 | 9126830 | 9126907 | 9122512 |
| PAQR6 | chr1 | − | 156245534 | 156245661 | 156245781 | 156245987 | 156244760 |
| MGEA5 | chr10 | − | 101800241 | 101800400 | 101803734 | 101804019 | 101798841 |
| ANK3 | chr10 | − | 60200128 | 60200227 | 60203001 | 60203100 | 60198339 |
| PITPNC1 | chr17 | + | 67532801 | 67532950 | 67552256 | 67552345 | 67377458 |
| MAPT | chr17 | + | 45962320 | 45962470 | 45971858 | 45971945 | 45894381 |
| VCAN | chr5 | + | 83519348 | 83522309 | 83537006 | 83542268 | 83512102 |
| DAB2IP | chr9 | + | 121759884 | 121760439 | 121763504 | 121763649 | 121758897 |
| PRKAG2 | chr7 | − | 151595344 | 151595454 | 151632068 | 151632138 | 151576370 |
| SETD4 | chr21 | − | 36048307 | 36048396 | 36053582 | 36053620 | 36045581 |
| SMARCA4 | chr19 | + | 11007901 | 11008023 | 11010380 | 11010531 | 11003339 |
| BAZ2B | chr2 | − | 159448241 | 159448403 | 159453612 | 159453801 | 159446781 |
| BAZ2B | chr2 | − | 159448241 | 159448409 | 159453612 | 159453801 | 159446781 |
| RBFA | chr18 | + | 80038504 | 80038617 | 80042134 | 80042219 | 80037329 |
| LUC7L | chr16 | − | 220648 | 220747 | 227241 | 227336 | 208077 |
| AIF1 | chr6 | + | 31615669 | 31615736 | 31616103 | 31616145 | 31615520 |
| PDE2A | chr11 | − | 72631077 | 72631139 | 72642253 | 72642326 | 72608661 |
| CAMK2G | chr10 | − | 73825278 | 73825347 | 73828088 | 73828121 | 73821677 |
| CAMK2G | chr10 | − | 73825278 | 73825347 | 73828088 | 73828121 | 73824039 |
| CCDC7 | chr10 | + | 32567669 | 32567891 | 32583033 | 32583307 | 32565557 |
| PMS1 | chr2 | + | 189863742 | 189863974 | 189867798 | 189867929 | 189854238 |
| PMS1 | chr2 | + | 189863742 | 189864228 | 189867798 | 189867929 | 189854238 |
| ADCK4 | chr19 | − | 40705324 | 40705447 | 40710058 | 40710136 | 40705095 |
| CRELD2 | chr22 | + | 49923233 | 49923317 | 49924359 | 49924455 | 49922611 |
| ARID4A | chr14 | + | 58360900 | 58361042 | 58364169 | 58365300 | 58359131 |
| MTDH | chr8 | + | 97699753 | 97699852 | 97706625 | 97706750 | 97690951 |
| MAP4K4 | chr2 | + | 101860824 | 101860986 | 101863820 | 101864051 | 101859735 |
| ACTR1B | chr2 | − | 97660570 | 97660646 | 97661881 | 97661946 | 97659351 |
| BCAS3 | chr17 | + | 61078332 | 61078529 | 61084466 | 61084564 | 61074919 |
| GTF2A2 | chr15 | − | 59650668 | 59650773 | 59652205 | 59652326 | 59642135 |
| C11orf80 | chr11 | + | 66788159 | 66788269 | 66796280 | 66796364 | 66759042 |
| ZNF106 | chr15 | − | 42442072 | 42442414 | 42444201 | 42444262 | 42439032 |
| ZNF106 | chr15 | − | 42448071 | 42448705 | 42466052 | 42466114 | 42446588 |
| SESN1 | chr6 | − | 108990644 | 108990835 | 108992786 | 108992899 | 108988542 |
| PHYHD1 | chr9 | + | 128940368 | 128940497 | 128940598 | 128940715 | 128937756 |
| GRID1 | chr10 | − | 85869009 | 85869180 | 85916185 | 85916239 | 85856028 |
| MAP9 | chr4 | − | 155355715 | 155355884 | 155357448 | 155357519 | 155355070 |
| NRCAM | chr7 | − | 108166920 | 108167073 | 108168276 | 108168402 | 108160360 |
| ASPDH | chr19 | − | 50512135 | 50512290 | 50512926 | 50513011 | 50511603 |
| VIPR1 | chr3 | + | 42532241 | 42532333 | 42534974 | 42535104 | 42531802 |
| PLEKHA6 | chr1 | − | 204250545 | 204250614 | 204251530 | 204251611 | 204249183 |
| ADAL | chr15 | + | 43334944 | 43335170 | 43335695 | 43335826 | 43333310 |
| C11orf74 | chr11 | + | 36636050 | 36636117 | 36648015 | 36648155 | 36633283 |
| SYMPK | chr19 | − | 45838460 | 45838615 | 45842249 | 45842489 | 45835077 |
| RANGAP1 | chr22 | − | 41274599 | 41274727 | 41280932 | 41281082 | 41268096 |
| PON2 | chr7 | − | 95412311 | 95412477 | 95416241 | 95416297 | 95411652 |
| ITSN2 | chr2 | − | 24275712 | 24275849 | 24286211 | 24286351 | 24271765 |
| SNW1 | chr14 | − | 77720710 | 77720828 | 77723180 | 77723277 | 77717952 |
| SPG11 | chr15 | − | 44585635 | 44585850 | 44589251 | 44589414 | 44583813 |
| RGL1 | chr1 | + | 183806374 | 183806485 | 183847565 | 183847774 | 183742125 |
| CCDC136 | chr7 | + | 128807359 | 128807545 | 128817757 | 128817864 | 128806687 |
| HACE1 | chr6 | − | 104833041 | 104833173 | 104843222 | 104843298 | 104811310 |
| SLC1A3 | chr5 | + | 36608328 | 36608604 | 36629449 | 36629587 | 36606354 |
| BRWD1 | chr21 | − | 39238478 | 39238573 | 39247700 | 39247832 | 39236594 |
| PSMB1 | chr6 | − | 170537233 | 170537340 | 170543600 | 170543730 | 170535116 |
| CUX2 | chr12 | + | 111291417 | 111291552 | 111293445 | 111293569 | 111263760 |
| VPS26A | chr10 | + | 69166041 | 69166110 | 69168488 | 69168631 | 69162405 |
| EGFL7 | chr9 | + | 136664691 | 136664785 | 136668240 | 136668362 | 136662929 |
| ADCK2 | chr7 | + | 140681041 | 140681137 | 140686989 | 140687241 | 140679154 |
| ZNF415 | chr19 | − | 53115209 | 53115307 | 53115703 | 53115811 | 53107878 |
| ANAPC16 | chr10 | + | 72223887 | 72224056 | 72230365 | 72230440 | 72220053 |
| CCM2 | chr7 | + | 45038252 | 45038426 | 45072725 | 45072783 | 45000184 |
| CCM2 | chr7 | + | 45069825 | 45069961 | 45072725 | 45072783 | 45064462 |
| ARL3 | chr10 | − | 102699372 | 102699489 | 102705345 | 102705489 | 102689892 |
| ARCN1 | chr11 | + | 118593589 | 118593698 | 118597706 | 118597911 | 118592708 |
| CALU | chr7 | + | 128754261 | 128754455 | 128754528 | 128754722 | 128748572 |

| | upstream | downstream | downstream | | | |
|---|---|---|---|---|---|---|
| geneSymbol | EE | ES | EE | PValue | FDR | type |
| FYN | 111696456 | 111702884 | 111703034 | 1.885E−04 | 9.613E−03 | UP |
| PIBF1 | 72931267 | 72998821 | 72998995 | 1.166E−03 | 2.928E−02 | DN |
| TBC1D5 | 17214370 | 17291894 | 17292001 | 1.919E−04 | 9.613E−03 | UP |
| TNRC6A | 24750813 | 24789231 | 24791817 | 1.441E−04 | 8.279E−03 | UP |
| SEZ6L | 26351243 | 26377672 | 26377775 | 1.717E−05 | 1.720E−03 | DN |
| SEZ6L | 26351243 | 26377672 | 26377775 | 1.054E−05 | 1.177E−03 | DN |
| ZMAT4 | 40532238 | 40697244 | 40697401 | 2.238E−04 | 1.054E−02 | UP |
| MOBP | 39467740 | 39502065 | 39502275 | 7.262E−04 | 2.234E−02 | NC |
| PDXDC1 | 14998405 | 15006393 | 15006583 | 1.167E−03 | 2.928E−02 | UP |
| TPM3 | 154170469 | 154172907 | 154172978 | 2.132E−03 | 4.360E−02 | DN |
| ANKRD24 | 4186461 | 4202025 | 4202090 | 1.222E−04 | 7.232E−03 | DN |
| WIPF2 | 40260667 | 40273789 | 40273999 | 9.676E−04 | 2.662E−02 | UP |
| ATRNL1 | 115302043 | 115394658 | 115394752 | 3.920E−05 | 3.126E−03 | UP |
| FXYD7 | 35148723 | 35151632 | 35151673 | 2.125E−03 | 4.360E−02 | DN |
| TSSC1 | 3214248 | 3354549 | 3354633 | 2.649E−05 | 2.300E−03 | DN |
| RP11-21J18.1 | 9122681 | 9134185 | 9134292 | 2.078E−08 | 9.020E−06 | DN |
| PAQR6 | 156244911 | 156246122 | 156246250 | 2.102E−03 | 4.360E−02 | NC |
| MGEA5 | 101799455 | 101806044 | 101806143 | 5.927E−04 | 1.946E−02 | DN |
| ANK3 | 60198537 | 60205791 | 60205890 | 1.169E−03 | 2.928E−02 | DN |
| PITPNC1 | 67378202 | 67553609 | 67553617 | 9.070E−04 | 2.553E−02 | UP |
| MAPT | 45894686 | 45978374 | 45978440 | 3.531E−04 | 1.385E−02 | UP |
| VCAN | 83512396 | 83545536 | 83545650 | 8.512E−05 | 5.636E−03 | DN |
| DAB2IP | 121758996 | 121763734 | 121763879 | 7.725E−04 | 2.322E−02 | DN |
| PRKAG2 | 151576452 | 151675419 | 151675637 | 1.856E−03 | 4.029E−02 | UP |
| SETD4 | 36046011 | 36057108 | 36057204 | 2.047E−03 | 4.331E−02 | UP |
| SMARCA4 | 11003397 | 11012948 | 11013112 | 4.371E−04 | 1.611E−02 | UP |
| BAZ2B | 159446975 | 159478574 | 159478721 | 8.836E−04 | 2.553E−02 | DN |
| BAZ2B | 159446975 | 159478574 | 159478721 | 2.074E−03 | 4.334E−02 | DN |
| RBFA | 80037506 | 80044211 | 80044285 | 2.782E−05 | 2.363E−03 | UP |
| LUC7L | 208188 | 229278 | 229450 | 1.468E−03 | 3.455E−02 | DN |
| AIF1 | 31615582 | 31616343 | 31616506 | 1.776E−03 | 3.965E−02 | DN |
| PDE2A | 72608751 | 72674136 | 72674453 | 2.505E−03 | 4.918E−02 | DN |
| CAMK2G | 73821730 | 73837467 | 73837511 | 3.550E−04 | 1.385E−02 | DN |
| CAMK2G | 73824084 | 73837467 | 73837511 | 4.716E−04 | 1.675E−02 | DN |
| CCDC7 | 32565620 | 32584231 | 32584304 | 2.051E−03 | 4.331E−02 | UP |
| PMS1 | 189855128 | 189873495 | 189873656 | 1.670E−03 | 3.799E−02 | UP |
| PMS1 | 189855128 | 189873495 | 189873656 | 1.156E−03 | 2.928E−02 | UP |
| ADCK4 | 40705181 | 40714066 | 40714133 | 1.397E−03 | 3.328E−02 | UP |
| CRELD2 | 49922707 | 49925416 | 49925557 | 3.157E−04 | 1.326E−02 | DN |
| ARID4A | 58359216 | 58365517 | 58365622 | 6.828E−05 | 5.033E−03 | UP |
| MTDH | 97691188 | 97713661 | 97713769 | 3.234E−04 | 1.330E−02 | DN |
| MAP4K4 | 101859864 | 101864929 | 101865036 | 4.741E−14 | 1.852E−10 | UP |
| ACTR1B | 97659477 | 97663842 | 97664107 | 2.880E−04 | 1.250E−02 | DN |
| BCAS3 | 61075020 | 61368326 | 61368494 | 9.910E−05 | 6.050E−03 | DN |
| GTF2A2 | 59642262 | 59657405 | 59657520 | 7.427E−04 | 2.267E−02 | UP |
| C11orf80 | 66759096 | 66800640 | 66800696 | 8.117E−06 | 1.035E−03 | UP |
| ZNF106 | 42439813 | 42444826 | 42444981 | 3.121E−04 | 1.325E−02 | UP |
| ZNF106 | 42446658 | 42472235 | 42472321 | 1.080E−03 | 2.850E−02 | DN |
| SESN1 | 108988687 | 108994461 | 108994609 | 1.367E−08 | 8.901E−06 | UP |
| PHYHD1 | 128937778 | 128941444 | 128941571 | 5.671E−04 | 1.894E−02 | DN |
| GRID1 | 85856190 | 86138818 | 86139024 | 2.530E−03 | 4.942E−02 | UP |
| MAP9 | 155355160 | 155360167 | 155360343 | 9.146E−04 | 2.553E−02 | DN |
| NRCAM | 108160492 | 108175321 | 108175357 | 2.062E−03 | 4.331E−02 | UP |
| ASPDH | 50511773 | 50513271 | 50513416 | 4.292E−04 | 1.597E−02 | UP |
| VIPR1 | 42531869 | 42535342 | 42535384 | 8.200E−05 | 5.621E−03 | DN |
| PLEKHA6 | 204249264 | 204255625 | 204255679 | 9.635E−05 | 5.975E−03 | DN |
| ADAL | 43333434 | 43336625 | 43336686 | 2.967E−06 | 4.830E−04 | UP |
| C11orf74 | 36633438 | 36659018 | 36659268 | 1.952E−03 | 4.167E−02 | DN |
| SYMPK | 45835228 | 45844029 | 45844200 | 3.337E−04 | 1.358E−02 | UP |
| RANGAP1 | 41268156 | 41285985 | 41286251 | 1.686E−03 | 3.808E−02 | DN |
| PON2 | 95411743 | 95424514 | 95424585 | 2.492E−03 | 4.916E−02 | UP |
| ITSN2 | 24271941 | 24293687 | 24293775 | 1.897E−04 | 9.613E−03 | DN |
| SNW1 | 77718530 | 77730987 | 77731129 | 1.557E−04 | 8.566E−03 | UP |
| SPG11 | 44584558 | 44592330 | 44592438 | 1.494E−03 | 3.480E−02 | UP |
| RGL1 | 183742289 | 183865995 | 183866073 | 1.531E−04 | 8.544E−03 | UP |
| CCDC136 | 128806858 | 128821798 | 128822119 | 1.938E−03 | 4.160E−02 | NC |
| HACE1 | 104811393 | 104849141 | 104849246 | 2.397E−03 | 4.778E−02 | UP |
| SLC1A3 | 36606735 | 36671028 | 36671233 | 1.826E−03 | 4.007E−02 | DN |
| BRWD1 | 39236784 | 39250795 | 39250889 | 1.048E−03 | 2.803E−02 | DN |
| PSMB1 | 170535405 | 170546102 | 170546184 | 4.232E−04 | 1.590E−02 | DN |
| CUX2 | 111263839 | 111295332 | 111295409 | 2.051E−04 | 1.001E−02 | DN |
| VPS26A | 69162512 | 69171155 | 69172856 | 2.218E−05 | 2.015E−03 | DN |
| EGFL7 | 136663108 | 136668556 | 136668673 | 4.938E−04 | 1.738E−02 | DN |
| ADCK2 | 140679283 | 140689596 | 140689725 | 3.581E−04 | 1.385E−02 | DN |
| ZNF415 | 53109908 | 53116312 | 53116433 | 1.046E−03 | 2.803E−02 | DN |
| ANAPC16 | 72220379 | 72233000 | 72235858 | 4.425E−04 | 1.616E−02 | UP |
| CCM2 | 45000363 | 45073459 | 45073571 | 6.462E−04 | 2.047E−02 | DN |
| CCM2 | 45064646 | 45073459 | 45073571 | 7.213E−04 | 2.234E−02 | NC |
| ARL3 | 102689943 | 102714272 | 102714407 | 1.037E−03 | 2.803E−02 | UP |
| ARCN1 | 118592856 | 118600624 | 118600993 | 2.491E−06 | 4.634E−04 | DN |
| CALU | 128748804 | 128758870 | 128759037 | 5.213E−05 | 4.073E−03 | DN |

TABLE 22
CARRIER_VS_TD_DE

| gene_symbol | log2FC | pval | padj | type |
|---|---|---|---|---|
| ADGRL4 | −2.288E+00 | 4.300E−07 | 1.837E−03 | DN |
| APOLD1 | −2.011E+00 | 5.150E−05 | 4.585E−02 | DN |
| BLOC1S5-TXNDC5 | 7.901E+00 | 8.228E−06 | 1.598E−02 | UP |
| C10orf10 | −2.265E+00 | 2.668E−05 | 2.715E−02 | DN |
| CD93 | −2.696E+00 | 1.217E−08 | 9.563E−05 | DN |
| ELOVL7 | −1.609E+00 | 5.627E−05 | 4.624E−02 | DN |
| F8A3 | −4.967E+00 | 3.446E−12 | 7.362E−08 | DN |
| FCGR3A | −2.307E+00 | 2.678E−06 | 6.357E−03 | DN |
| FER1L6 | 4.108E+00 | 4.934E−05 | 4.584E−02 | UP |
| ITGA6 | −1.687E+00 | 1.571E−05 | 2.085E−02 | DN |
| KIF25 | 3.740E+00 | 1.363E−05 | 2.081E−02 | UP |
| MIR143HG | 2.020E+00 | 2.449E−05 | 2.616E−02 | UP |
| NUTM2E | 4.145E+00 | 4.160E−06 | 8.888E−03 | UP |
| OR7D2 | −3.348E+00 | 5.521E−05 | 4.624E−02 | DN |
| PECAM1 | −2.068E+00 | 2.112E−05 | 2.375E−02 | DN |
| POC1B-GALNT4 | 7.515E+00 | 3.775E−05 | 3.666E−02 | UP |
| RP11-17M24.3 | 7.811E+00 | 1.314E−05. | 2.081E−02 | UP |
| RP11-73M18.2 | −8.367E+00 | 8.424E−07 | 2.250E−03 | DN |
| RP11-986E7.7 | −7.624E+00 | 1.659E−05 | 2.085E−02 | DN |
| RPL17-C18orf32 | −3.080E+00 | 5.401E−07 | 1.855E−03 | DN |
| S100A9 | −4.195E+00 | 1.343E−08 | 9.563E−05 | DN |
| SST | 2.449E+00 | 1.507E−05 | 2.085E−02 | UP |
| TBC1D3L | 2.133E+00 | 6.079E−07 | 1.855E−03 | UP |
| TVP23C-CDRT4 | −8.931E+00 | 2.301E−08 | 1.229E−04 | DN |
| VSIG4 | −2.411E+00 | 9.151E−06 | 1.629E−02 | DN |
| XXbac-BPG246D15.9 | 7.849E+00 | 2.059E−05 | 2.375E−02 | UP |

TABLE 23
GO CARRIER VS TD

| GO: SE UP | | | | | | | |
|---|---|---|---|---|---|---|---|
| ID | Description | GeneRatio | BgRatio | pvalue | p.adjust | qvalue | Count |
| GO: 0048667 | cell morphogenesis involved in neuron differentiation | 21/182 | 500/17913 | 4.029E−08 | 1.062E−04 | 9.819E−05 | 21 |
| GO: 0050807 | regulation of synapse organization | 13/182 | 218/17913 | 3.665E−07 | 2.527E−04 | 2.336E−04 | 13 |
| GO: 0010769 | regulation of cell morphogenesis involved in differentiation | 14/182 | 260/17913 | 4.462E−07 | 2.527E−04 | 2.336E−04 | 14 |
| GO: 0050803 | regulation of synapse structure or activity | 13/182 | 222/17913 | 4.512E−07 | 2.527E−04 | 2.336E−04 | 13 |
| GO: 0007030 | Golgi organization | 9/182 | 94/17913 | 4.794E−07 | 2.527E−04 | 2.336E−04 | 9 |
| GO: 0050808 | synapse organization | 17/182 | 394/17913 | 5.865E−07 | 2.577E−04 | 2.382E−04 | 17 |
| GO: 0051056 | regulation of small GTPase mediated signal transduction | 15/182 | 316/17913 | 8.510E−07 | 3.205E−04 | 2.962E−04 | 15 |
| GO: 0048814 | regulation of dendrite morphogenesis | 8/182 | 82/17913 | 1.828E−06 | 5.353E−04 | 4.949E−04 | 8 |
| GO: 0030010 | establishment of cell polarity | 9/182 | 126/17913 | 5.619E−06 | 1.234E−03 | 1.141E−03 | 9 |
| GO: 0099175 | regulation of postsynapse organization | 7/182 | 98/17913 | 6.326E−05 | 8.068E−03 | 7.458E−03 | 7 |
| GO: 0051651 | maintenance of location in cell | 6/182 | 72/17913 | 9.078E−05 | 1.040E−02 | 9.618E−03 | 6 |
| GO: 0051493 | regulation of cytoskeleton organization | 15/182 | 472/17913 | 9.893E−05 | 1.087E−02 | 1.005E−02 | 15 |
| GO: 0099173 | postsynapse organization | 8/182 | 158/17913 | 2.145E−04 | 2.146E−02 | 1.983E−02 | 8 |
| GO: 0031532 | actin cytoskeleton reorganization | 6/182 | 85/17913 | 2.279E−04 | 2.146E−02 | 1.983E−02 | 6 |
| GO: 0106027 | neuron projection organization | 6/182 | 85/17913 | 2.279E−04 | 2.146E−02 | 1.983E−02 | 6 |
| GO: 0043087 | regulation of GTPase activity | 13/182 | 410/17913 | 2.964E−04 | 2.441E−02 | 2.257E−02 | 13 |
| GO: 0098901 | regulation of cardiac muscle cell action potential | 4/182 | 34/17913 | 3.768E−04 | 2.921E−02 | 2.700E−02 | 4 |
| GO: 0051656 | establishment of organelle localization | 13/182 | 448/17913 | 6.832E−04 | 4.502E−02 | 4.162E−02 | 13 |
| GO: 0043547 | positive regulation of GTPase activity | 11/182 | 339/17913 | 7.157E−04 | 4.602E−02 | 4.254E−02 | 11 |
| GO: 0035637 | multicellular organismal signaling | 8/182 | 193/17913 | 8.142E−04 | 4.769E−02 | 4.409E−02 | 8 |

| GO: SE UP | |
|---|---|
| ID | geneID |
| GO: 0048667 | SHC1/LRP8/RBFOX2/ABI2/PTK2/SPP1/NFASC/DBN1/ANK3/PARP6/MARK2/BRSK2/ADGRB3/RAPGEF2/ |
| | DNM1L/NRCAM/NFIB/SIPA1L1/PDLIM7/FN1/SOS1 |
| GO: 0050807 | LRP8/DGKB/ABI2/PTK2/DBN1/ADGRB2/ADGRB3/DNM1L/NRCAM/DCTN1/SIPA1L1/GPM6A/YWHAZ |
| GO: 0010769 | LRP8/ABI2/PTK2/SPP1/DBN1/PARP6/MARK2/BRSK2/ADGRB3/RAPGEF2/DNM1L/NRCAM/SIPA1L1/FN1 |
| GO: 0050803 | LRP8/DGKB/ABI2/PTK2/DBN1/ADGRB2/ADGRB3/DNM1L/NRCAM/DCTN1/SIPA1L1/GPM6A/YWHAZ |
| GO: 0007030 | COG1/TRAPPC12/SYNE1/BCAS3/MYO18A/CIT/STX16/ARHGAP21/YWHAZ |
| GO: 0050808 | NRXN2/LRP8/DGKB/ABI2/PTK2/NFASC/DBN1/ANK3/ADGRB2/ADGRB3/DNM1L/NRCAM/DCTN1/ |
| | SIPA1L1/KIRREL3/GPM6A/YWHAZ |
| GO: 0051056 | SHC1/ADCYAP1R1/MADD/RHOT1/ARHGEF2/ARHGEF9/MAP4K4/ARHGEF25/NF1/ARHGAP12/ITSN2/ |
| | SIPA1L1/RHOC/SOS1/ARHGAP21 |
| GO: 0048814 | LRP8/ABI2/DBN1/PARP6/ADGRB3/RAPGEF2/DNM1L/SIPA1L1 |
| GO: 0030010 | NSFL1C/PTK2/ARHGEF2/MARK2/BRSK2/BCAS3/MYO18A/DCTN1/GSN |
| GO: 0099175 | LRP8/DGKB/ABI2/DBN1/DNM1L/NRCAM/SIPA1L1 |
| GO: 0051651 | DBN1/ANK3/SYNE1/RANGAP1/GSN/ARHGAP21 |
| GO: 0051493 | NSFL1C/STAG2/ABI2/PTK2/ARHGEF2/MARK2/BIN1/GPM6B/BCAS3/CIT/PHLDB1/DCTN1/CYLD/RHOC/GSN |
| GO: 0099173 | NRXN2/LRP8/DGKB/ABI2/DBN1/DNM1L/NRCAM/SIPA1L1 |
| GO: 0031532 | SHC1/CDC42BPA/BRSK2/BCAS3/SIPA1L1/GSN |
| GO: 0106027 | LRP8/ABI2/DBN1/DNM1L/DCTN1/SIPA1L1 |
| GO: 0043087 | PTK2/MAP4K4/ADGRB3/RAPGEF2/NF1/DNM1L/BCAS3/ARHGAP12/WNK1/RANGAP1/SIPA1L1/SOS1/ARHGAP21 |
| GO: 0098901 | ANK3/CAMK2D/BIN1/ANK2 |
| GO: 0051656 | NSFL1C/PTK2/RHOT1/KIF13A/ARHGEF2/TRAPPC12/DNM1L/CPLX2/BLOC1S6/PPP6R3/DCTN1/ARHGAP21/YWHAZ |
| GO: 0043547 | MAP4K4/ADGRB3/RAPGEF2/NF1/DNM1L/BCAS3/ARHGAP12/RANGAP1/SIPA1L1/SOS1/ARHGAP21 |
| GO: 0035637 | NFASC/ANK3/CAMK2D/BIN1/ANK2/NRCAM/ASPH/ATP2B2 |
| GO: 0048667 | SHC1/LRP8/RBFOX2/ABI2/PTK2/SPP1/NFASC/DBN1/ANK3/PARP6/MARK2/BRSK2/ADGRB3/RAPGEF2/ |
| | DNM1L/NRCAM/NFIB/SIPA1L1/PDLIM7/FN1/SOS1 |
| GO: 0050807 | LRP8/DGKB/ABI2/PTK2/DBN1/ADGRB2/ADGRB3/DNM1L/NRCAM/DCTN1/SIPA1L1/GPM6A/YWHAZ |
| GO: 0010769 | LRP8/ABI2/PTK2/SPP1/DBN1/PARP6/MARK2/BRSK2/ADGRB3/RAPGEF2/DNM1L/NRCAM/SIPA1L1/FN1 |
| GO: 0050803 | LRP8/DGKB/ABI2/PTK2/DBN1/ADGRB2/ADGRB3/DNM1L/NRCAM/DCTN1/SIPA1L1/GPM6A/YWHAZ |
| GO: 0007030 | COG1/TRAPPC12/SYNE1/BCAS3/MYO18A/CIT/STX16/ARHGAP21/YWHAZ |
| GO: 0050808 | NRXN2/LRP8/DGKB/ABI2/PTK2/NFASC/DBN1/ANK3/ADGRB2/ADGRB3/DNM1L/NRCAM/DCTN1/ |
| | SIPA1L1/KIRREL3/GPM6A/YWHAZ |
| GO: 0051056 | SHC1/ADCYAP1R1/MADD/RHOT1/ARHGEF2/ARHGEF9/MAP4K4/ARHGEF25/NF1/ARHGAP12/ITSN2/ |
| | SIPA1L1/RHOC/SOS1/ARHGAP21 |
| GO: 0048814 | LRP8/ABI2/DBN1/PARP6/ADGRB3/RAPGEF2/DNM1L/SIPA1L1 |
| GO: 0030010 | NSFL1C/PTK2/ARHGEF2/MARK2/BRSK2/BCAS3/MYO18A/DCTN1/GSN |
| GO: 0099175 | LRP8/DGKB/ABI2/DBN1/DNM1L/NRCAM/SIPA1L1 |
| GO: 0051651 | DBN1/ANK3/SYNE1/RANGAP1/GSN/ARHGAP21 |
| GO: 0051493 | NSFL1C/STAG2/ABI2/PTK2/ARHGEF2/MARK2/BIN1/GPM6B/BCAS3/CIT/PHLDB1/DCTN1/CYLD/ |
| | RHOC/GSN |
| GO: 0099173 | NRXN2/LRP8/DGKB/ABI2/DBN1/DNM1L/NRCAM/SIPA1L1 |
| GO: 0031532 | SHC1/CDC42BPA/BRSK2/BCAS3/SIPA1L1/GSN |
| GO: 0106027 | LRP8/ABI2/DBN1/DNM1L/DCTN1/SIPA1L1/ |
| GO: 0043087 | SOS1/ARHGAP21 |
| | PTK2/MAP4K4/ADGRB3/RAPGEF2/NF1/DNM1L/BCAS3/ARHGAP12/WNK1/RANGAP1/SIPA1L1 |
| GO: 0098901 | ANK3/CAMK2D/BIN1/ANK2 |
| GO: 0051656 | NSFL1C/PTK2/RHOT1/KIF13A/ARHGEF2/TRAPPC12/DNM1L/CPLX2/BLOC1S6/PPP6R3/DCTN1/ |
| | ARHGAP21/YWHAZ |
| GO: 0043547 | MAP4K4/ADGRB3/RAPGEF2/NF1/DNM1L/BCAS3/ARHGAP12/RANGAP1/SIPA1L1/SOS1/ARHGAP21 |
| GO: 0035637 | NFASC/ANK3/CAMK2D/BIN1/ANK2/NRCAM/ASPH/ATP2B2 |

| GO: SE DOWN | | | | | | | |
|---|---|---|---|---|---|---|---|
| ID | Description | GeneRatio | BgRatio | pvalue | p.adjust | qvalue | Count |
| GO: 0098901 | regulation of cardiac muscle cell action potential | 5/131 | 34/17913 | 4.547E−06 | 6.754E−03 | 6.084E−03 | 5 |
| GO: 0050808 | synapse organization | 13/131 | 394/17913 | 6.442E−06 | 6.754E−03 | 6.084E−03 | 13 |
| GO: 0046628 | positive regulation of insulin receptor signaling pathway | 4/131 | 20/17913 | 1.209E−05 | 6.754E−03 | 6.084E−03 | 4 |
| GO: 0010959 | regulation of metal ion transport | 12/131 | 365/17913 | 1.541E−05 | 6.754E−03 | 6.084E−03 | 12 |
| GO: 0048667 | cell morphogenesis involved in neuron differentiation | 14/131 | 500/17913 | 1.796E−05 | 6.754E−03 | 6.084E−03 | 14 |
| GO: 1900078 | positive regulation of cellular response to insulin stimulus | 4/131 | 22/17913 | 1.804E−05 | 6.754E−03 | 6.084E−03 | 4 |
| GO: 0051648 | vesicle localization | 10/131 | 270/17913 | 2.981E−05 | 8.370E−03 | 7.539E−03 | 10 |
| GO: 1902305 | regulation of sodium ion transmembrane transport | 5/131 | 51/17913 | 3.475E−05 | 8.672E−03 | 7.812E−03 | 5 |
| GO: 0072659 | protein localization to plasma membrane | 9/131 | 237/17913 | 6.224E−05 | 1.271E−02 | 1.145E−02 | 9 |
| GO: 0022604 | regulation of cell morphogenesis | 12/131 | 430/17913 | 7.580E−05 | 1.419E−02 | 1.278E−02 | 12 |
| GO: 0051650 | establishment of vesicle localization | 9/131 | 253/17913 | 1.026E−04 | 1.700E−02 | 1.532E−02 | 9 |
| GO: 0051656 | establishment of organelle localization | 12/131 | 448/17913 | 1.117E−04 | 1.700E−02 | 1.532E−02 | 12 |
| GO: 0050775 | positive regulation of dendrite morphogenesis | 4/131 | 35/17913 | 1.200E−04 | 1.700E−02 | 1.532E−02 | 4 |
| GO: 0071346 | cellular response to interferon-gamma | 7/131 | 153/17913 | 1.332E−04 | 1.760E−02 | 1.585E−02 | 7 |
| GO: 0099173 | postsynapse organization | 7/131 | 158/17913 | 1.626E−04 | 2.029E−02 | 1.827E−02 | 7 |
| GO: 1990778 | protein localization to cell periphery | 9/131 | 276/17913 | 1.974E−04 | 2.127E−02 | 1.916E−02 | 9 |
| GO: 0086001 | cardiac muscle cell action potential | 5/131 | 74/17913 | 2.084E−04 | 2.127E−02 | 1.916E−02 | 5 |
| GO: 0007018 | microtubule-based movement | 8/131 | 224/17913 | 2.443E−04 | 2.286E−02 | 2.059E−02 | 8 |
| GO: 0007163 | establishment or maintenance of cell polarity | 7/131 | 172/17913 | 2.738E−04 | 2.450E−02 | 2.207E−02 | 7 |
| GO: 0034341 | response to interferon-gamma | 7/131 | 173/17913 | 2.836E−04 | 2.450E−02 | 2.207E−02 | 7 |
| GO: 0061564 | axon development | 11/131 | 438/17913 | 3.765E−04 | 3.020E−02 | 2.721E−02 | 11 |
| GO: 0070838 | divalent metal ion transport | 11/131 | 454/17913 | 5.086E−04 | 3.360E−02 | 3.026E−02 | 11 |
| GO: 0001954 | positive regulation of cell-matrix adhesion | 4/131 | 51/17913 | 5.232E−04 | 3.360E−02 | 3.026E−02 | 4 |
| GO: 0060333 | interferon-gamma-mediated signaling pathway | 5/131 | 91/17913 | 5.448E−04 | 3.360E−02 | 3.026E−02 | 5 |
| GO: 0034453 | microtubule anchoring | 3/131 | 23/17913 | 6.082E−04 | 3.437E−02 | 3.096E−02 | 3 |
| GO: 0010970 | transport along microtubule | 6/131 | 143/17913 | 6.428E−04 | 3.437E−02 | 3.096E−02 | 6 |
| GO: 0099111 | microtubule-based transport | 6/131 | 143/17913 | 6.428E−04 | 3.437E−02 | 3.096E−02 | 6 |
| GO: 0042982 | amyloid precursor protein metabolic process | 4/131 | 55/17913 | 6.981E−04 | 3.564E−02 | 3.210E−02 | 4 |
| GO: 0030705 | cytoskeleton-dependent intracellular transport | 6/131 | 154/17913 | 9.467E−04 | 4.472E−02 | 4.028E−02 | 6 |
| GO: 0060560 | developmental growth involved in morphogenesis | 7/131 | 214/17913 | 1.006E−03 | 4.611E−02 | 4.154E−02 | 7 |

| GO: SE DOWN | |
|---|---|
| ID | geneID |
| GO: 0098901 | ANK3/DLG1/CAMK2D/BIN1/SLMAP |
| GO: 0050808 | NRXN2/PTPRS/ACTR2/PCDHGC3/PALM/SEMA4D/ANK3/DLG1/SPARCL1/CAMK2B/ARHGAP39/TNC/OPA1 |
| GO: 0046628 | GKAP1/SORBS1/PRKCZ/OPA1 |
| GO: 0010959 | ATP2B1/CAMK2A/ANK3/DLG1/CAMK2D/BIN1/WNK2/CAMK2G/CAMK2B/GSTO1/SLMAP/BDKRB1 |
| GO: 0048667 | PTPRS/ACTR2/GAB1/CAMK2A/ILK/SEMA4D/ANK3/NCAM1/BRSK2/CAMK2B/CHL1/NRXN3/OPA1/NIN |
| GO: 1900078 | GKAP1/SORBS1/PRKCZ/OPA1 |
| GO: 0051648 | MLPH/TFG/MCFD2/CAMK2A/DYNC1I1/CADPS/BRSK2/KIF3A/CLASP2/PRKCZ |
| GO: 1902305 | ANK3/DLG1/CAMK2D/WNK2/SLMAP |
| GO: 0072659 | EPB41L3/PALM/ANK3/DLG1/FLOT2/SORBS1/CLASP2/SLMAP/PRKCZ |
| GO: 0022604 | EPB41L3/PTPRS/ACTR2/PALM/ILK/SEMA4D/DLG1/BRSK2/CAMK2B/UNC13A/OPA1/NIN |
| GO: 0051650 | MLPH/TFG/MCFD2/CAMK2A/DYNC1I1/CADPS/KIF3A/CLASP2/PRKCZ |
| GO: 0051656 | MLPH/TFG/ACTR2/MCFD2/CAMK2A/DLG1/DYNC1I1/CADPS/KIF3A/CLASP2/PRKCZ/OPA1 |
| GO: 0050775 | ACTR2/ILK/CAMK2B/OPA1 |
| GO: 0071346 | ACTR2/CAMK2A/NCAM1/CAMK2D/CAMK2G/CAMK2B/SYNCRIP |
| GO: 0099173 | NRXN2/PTPRS/ACTR2/DLG1/CAMK2B/ARHGAP39/OPA1 |
| GO: 1990778 | EPB41L3/PALM/ANK3/DLG1/FLOT2/SORBS1/CLASP2/SLMAP/PRKCZ |
| GO: 0086001 | ANK3/DLG1/CAMK2D/BIN1/SLMAP |
| GO: 0007018 | RPGR/ACTR2/DYNC1I1/FLOT2/KLC1/KIF3A/PRKCZ/OPA1 |
| GO: 0007163 | ACTR2/ILK/DLG1/FLOT2/BRSK2/CLASP2/PRKCZ |
| GO: 0034341 | ACTR2/CAMK2A/NCAM1/CAMK2D/CAMK2G/CAMK2B/SYNCRIP |
| GO: 0061564 | PTPRS/GAB1/ILK/SEMA4D/ANK3/NCAM1/BRSK2/CHL1/NRXN3/TNC/NIN |
| GO: 0070838 | ATP2B1/CAMK2A/CAMK2D/BIN1/CAMK2G/CAMK2B/CNNM2/GSTO1/OPA1/BDKRB1/MICU3 |
| GO: 0001954 | ILK/MAP4K4/COL16A1/PRKCZ |
| GO: 0060333 | CAMK2A/NCAM1/CAMK2D/CAMK2G/CAMK2B |
| GO: 0034453 | KIF3A/CLASP2/NIN |
| GO: 0010970 | RPGR/DYNC1I1/FLOT2/KIF3A/PRKCZ/OPA1 |
| GO: 0099111 | RPGR/DYNC1I1/FLOT2/KIF3A/PRKCZ/OPA1 |
| GO: 0042982 | DLG1/BIN1/FLOT2/UNC13A |
| GO: 0030705 | RPGR/DYNC1I1/FLOT2/KIF3A/PRKCZ/OPA1 |
| GO: 0060560 | PTPRS/ILK/SEMA4D/UNC13A/TNC/PRKCZ/NIN |

TABLE 24

| | | | | |
|---|---|---|---|---|
| gene_symbol | log2FC | pval | padj | type |

FXS_VS_TD_DE (Part 1)

| | | | | |
|---|---|---|---|---|
| ACAN | −2.399E+00 | 6.244E−05 | 1.944E−02 | DN |
| ADAMTS1 | −2.174E+00 | 1.609E−06 | 1.412E−03 | DN |
| ADIRF | −1.963E+00 | 6.619E−05 | 2.026E−02 | DN |
| ANXA2 | −2.272E+00 | 8.559E−06 | 4.027E−03 | DN |
| C11orf96 | −3.886E+00 | 1.642E−06 | 1.412E−03 | DN |
| C2CD4B | −4.041E+00 | 1.340E−04 | 3.560E−02 | DN |
| CCL2 | −4.885E+00 | 7.357E−06 | 4.026E−03 | DN |
| CD93 | −3.261E+00 | 1.038E−05 | 4.360E−03 | DN |
| CDKN1A | −1.948E+00 | 8.934E−06 | 4.027E−03 | DN |
| CEBPD | −1.935E+00 | 6.093E−06 | 3.438E−03 | DN |
| COL8A1 | −4.053E+00 | 5.677E−05 | 1.831E−02 | DN |
| CTB-43P18.3 | 2.239E+00 | 1.476E−07 | 2.080E−04 | UP |
| CTD-2410N18.5 | 6.900E+00 | 5.136E−09 | 1.159E−05 | UP |
| CXCL2 | −5.482E+00 | 3.362E−09 | 8.674E−06 | DN |
| CXCL3 | −6.207E+00 | 6.922E−09 | 1.389E−05 | DN |
| CXCL8 | −7.796E+00 | 1.866E−04 | 4.435E−02 | DN |
| CYR61 | −2.716E+00 | 5.254E−07 | 5.930E−04 | DN |
| ERAP2 | −3.383E+00 | 1.666E−09 | 8.674E−06 | DN |
| FOS | −1.295E+00 | 1.863E−04 | 4.435E−02 | DN |
| FSTL1 | −1.529E+00 | 1.068E−04 | 2.968E−02 | DN |
| GABRE | −3.430E+00 | 2.268E−06 | 1.781E−03 | DN |
| GADD45A | −1.671E+00 | 8.399E−06 | 4.027E−03 | DN |
| GADD45B | −2.120E+00 | 2.097E−09 | 8.674E−06 | DN |
| GBP2 | −2.117E+00 | 2.251E−05 | 8.468E−03 | DN |
| GPRC5A | −4.584E+00 | 8.196E−06 | 4.027E−03 | DN |
| HILPDA | −2.106E+00 | 2.139E−06 | 1.756E−03 | DN |

FXS_VS_TD_DE (Part 2)

| | | | | |
|---|---|---|---|---|
| HLA-B | −1.304E+00 | 5.928E−05 | 1.878E−02 | DN |
| HLA-DQB1 | −2.311E+00 | 4.501E−06 | 2.803E−03 | DN |
| HSPG2 | −1.620E+00 | 7.873E−05 | 2.331E−02 | DN |
| ICAM1 | −3.814E+00 | 9.365E−06 | 4.027E−03 | DN |
| IER3 | −2.386E+00 | 2.460E−09 | 8.674E−06 | DN |
| IFI16 | −1.902E+00 | 1.697E−04 | 4.255E−02 | DN |
| IL1R1 | −2.586E+00 | 4.536E−08 | 7.446E−05 | DN |
| IL32 | −1.934E+00 | 3.521E−06 | 2.293E−03 | DN |
| ITGA5 | −2.854E+00 | 1.172E−04 | 3.160E−02 | DN |
| KIAA0040 | −2.202E+00 | 3.707E−05 | 1.287E−02 | DN |
| KLF6 | −1.305E+00 | 1.532E−04 | 3.953E−02 | DN |
| LIPG | −5.086E+00 | 9.001E−05 | 2.580E−02 | DN |
| LMOD1 | −3.975E+00 | 3.236E−06 | 2.247E−03 | DN |
| LRRC32 | −1.592E+00 | 3.555E−06 | 2.293E−03 | DN |
| MAFF | −2.437E+00 | 9.893E−07 | 9.925E−04 | DN |
| MDK | −2.380E+00 | 1.068E−04 | 2.968E−02 | DN |
| MSC | −2.298E+00 | 1.322E−06 | 1.257E−03 | DN |
| MTHFD2 | −1.847E+00 | 1.532E−04 | 3.953E−02 | DN |
| MXRA8 | −2.235E+00 | 1.610E−04 | 4.094E−02 | DN |
| NPIPA8 | 4.902E+00 | 3.450E−05 | 1.221E−02 | UP |
| PDLIM1 | −4.010E+00 | 1.809E−11 | 3.267E−07 | DN |
| PECAM1 | −1.493E+00 | 1.397E−05 | 5.608E−03 | DN |
| PLA1A | −3.200E+00 | 3.801E−08 | 6.865E−05 | DN |
| RMST | −1.964E+00 | 4.404E−05 | 1.473E−02 | DN |
| RP11-133K1.2 | 7.998E+00 | 9.277E−06 | 4.027E−03 | UP |
| RP11-219A15.4 | 5.012E+00 | 8.712E−07 | 9.255E−04 | UP |

FXS_VS_TD_DE (Part 3)

| | | | | |
|---|---|---|---|---|
| RP11-383H13.1 | −2.169E+00 | 2.872E−05 | 1.058E−02 | DN |
| RP11-514P8.8 | 7.396E+00 | 1.849E−04 | 4.435E−02 | UP |
| RP11-561023.5 | −3.220E+00 | 8.993E−06 | 4.027E−03 | DN |
| RP5-1028K7.3 | −8.372E+00 | 2.421E−06 | 1.821E−03 | DN |
| RSPO2 | 1.641E+00 | 1.553E−05 | 6.097E−03 | UP |
| S100A6 | −1.445E+00 | 8.117E−05 | 2.364E−02 | DN |
| S100A8 | −4.574E+00 | 3.417E−05 | 1.221E−02 | DN |
| SCN1A | 1.341E+00 | 2.097E−04 | 4.795E−02 | UP |
| SECTM1 | −4.357E+00 | 5.588E−05 | 1.831E−02 | DN |
| SERPINE1 | −4.530E+00 | 2.439E−07 | 3.146E−04 | DN |
| SERPING1 | −1.821E+00 | 2.017E−04 | 4.669E−02 | DN |
| SOCS3 | −3.914E+00 | 2.045E−05 | 7.858E−03 | DN |
| SV2C | 1.794E+00 | 1.211E−05 | 4.969E−03 | UP |
| TAGLN2 | −1.885E+00 | 2.937E−06 | 2.122E−03 | DN |
| TFPI | −2.528E+00 | 1.796E−04 | 4.435E−02 | DN |
| TIMP3 | −1.573E+00 | 1.990E−04 | 4.667E−02 | DN |
| TM4SF1 | −1.873E+00 | 1.497E−07 | 2.080E−04 | DN |
| TMEM233 | −3.019E+00 | 7.884E−06 | 4.027E−03 | DN |
| TNC | −2.894E+00 | 4.938E−06 | 2.973E−03 | DN |
| TNFAIP3 | −2.596E+00 | 8.341E−06 | 4.027E−03 | DN |
| TNFRSF12A | −3.853E+00 | 6.011E−06 | 3.438E−03 | DN |
| TUBA1C | −1.520E+00 | 4.072E−05 | 1.387E−02 | DN |
| TUBB6 | −2.943E+00 | 3.644E−10 | 3.290E−06 | DN |
| VCAM1 | 2.540E+00 | 7.314E−05 | 2.201E−02 | DN |
| YBX3 | −2.036E+00 | 3.062E−09 | 8.674E−06 | DN |
| ZFP36 | −2.647E+00 | 1.149E−04 | 3.143E−02 | DN |

TABLE 25
FXS_VS_TD_SE

| | | | exonStart_{—} | exon | upstream | ups tream |
|---|---|---|---|---|---|---|
| geneSymbol | chr | strand | 0base | End | ES | EE |
| EXOC7 | chr17 | − | 76086853 | 76086892 | 76086079 | 76086145 |
| EXOC7 | chr17 | − | 76091142 | 76091235 | 76089174 | 76089320 |
| GRIPAP1 | chrX | − | 48981794 | 48981872 | 48981595 | 48981691 |
| EPB41L3 | chr18 | − | 5407700 | 5407736 | 5406776 | 5406968 |
| BANF1 | chr11 | + | 66003234 | 66003373 | 66002078 | 66002570 |
| JOSD2 | chr19 | − | 50507573 | 50507699 | 50506377 | 50506572 |
| SLC25A26 | chr3 | + | 66236543 | 66236700 | 66220741 | 66221127 |
| ARHGAP23 | chr17 | + | 38498931 | 38498954 | 38498413 | 38498510 |
| CLIP2 | chr7 | + | 74372931 | 74373036 | 74364254 | 74364315 |
| SH3BP5 | chr3 | − | 15256852 | 15257113 | 15254852 | 15256303 |
| IL17RC | chr3 | + | 9932819 | 9932858 | 9932607 | 9932703 |
| EEF1D | chr8 | − | 143588990 | 143590081 | 143586728 | 143586852 |
| EEF1D | chr8 | − | 143588990 | 143590092 | 143586728 | 143586852 |
| SAT2 | chr17 | − | 7626942 | 7627044 | 7626752 | 7626793 |
| RGS14 | chr5 | + | 177371474 | 177371589 | 177371349 | 177371396 |
| RNF14 | chr5 | + | 141978302 | 141978830 | 141974803 | 141974955 |
| CMC2 | chr16 | − | 81001247 | 81001334 | 80998284 | 80998354 |
| BCAS4 | chr20 | + | 50875996 | 50876093 | 50841765 | 50841900 |
| C1orf61 | chr1 | − | 156407841 | 156407975 | 156404256 | 156404601 |
| KIF21A | chr12 | − | 39346465 | 39346504 | 39342033 | 39342124 |
| LRRFIP2 | chr3 | − | 37066223 | 37066325 | 37065809 | 37065942 |
| LRRFIP2 | chr3 | − | 37072789 | 37072882 | 37065809 | 37065942 |
| CABP1 | chr12 | + | 120656092 | 120656272 | 120650084 | 120650682 |
| DNM2 | chr19 | + | 10808568 | 10808580 | 10805915 | 10805967 |
| ARHGEF1 | chr19 | + | 41906456 | 41906620 | 41905938 | 41906025 |
| ABTB1 | chr3 | + | 127676966 | 127677083 | 127676535 | 127676581 |
| PLXNB3 | chrX | + | 153778260 | 153778325 | 153777947 | 153778095 |
| ACTR2 | chr2 | + | 65242048 | 65242063 | 65239851 | 65239962 |
| ATP6V0A1 | chr17 | + | 42508571 | 42508589 | 42507519 | 42507627 |
| APMAP | chr20 | − | 24968891 | 24969084 | 24962924 | 24964022 |
| CHGA | chr14 | + | 92931249 | 92931702 | 92929716 | 92929815 |
| ZFAND5 | chr9 | − | 72363469 | 72363606 | 72360627 | 72360787 |
| RPS24 | chr10 | + | 78037964 | 78037982 | 78037193 | 78037304 |
| RPS24 | chr10 | + | 78037964 | 78037982 | 78037438 | 78037441 |
| RALYL | chr8 | + | 84804769 | 84804802 | 84774578 | 84774654 |
| TXNL4A | chr18 | − | 79977597 | 79977701 | 79970810 | 79973856 |
| SNAP25 | chr20 | + | 10292881 | 10292999 | 10284723 | 10284772 |
| SNAP25 | chr20 | + | 10293160 | 10293278 | 10292881 | 10292999 |
| LUC7L2 | chr7 | + | 139374400 | 139374471 | 139359845 | 139360322 |
| TPM1 | chr15 | + | 63061712 | 63061788 | 63061197 | 63061273 |
| MTMR3 | chr22 | + | 30023456 | 30023483 | 30022608 | 30022697 |
| BID | chr22 | − | 17750104 | 17750174 | 17739348 | 17739488 |
| TPD52L2 | chr20 | + | 63887563 | 63887605 | 63882718 | 63882820 |
| MVK | chr12 | + | 109579801 | 109579946 | 109575997 | 109576145 |
| PLEKHJ1 | chr19 | − | 2234149 | 2234240 | 2233148 | 2234061 |
| TM7SF2 | chr11 | + | 65115313 | 65115394 | 65114912 | 65115081 |
| PTK2 | chr8 | − | 140669726 | 140669735 | 140668268 | 140668424 |
| ZFYVE19 | chr15 | + | 40812698 | 40812902 | 40810648 | 40810757 |
| SRM | chr1 | − | 11055780 | 11055926 | 11054961 | 11055084 |
| PPP5C | chr19 | + | 46383410 | 46383476 | 46376452 | 46376574 |
| AP2B1 | chr17 | + | 35670856 | 35670898 | 35657598 | 35657791 |
| RBM42 | chr19 | + | 35632935 | 35633001 | 35631330 | 35631405 |
| TTYH1 | chr19 | + | 54435827 | 54435873 | 54435541 | 54435684 |
| MARK4 | chr19 | + | 45298142 | 45298222 | 45297675 | 45297954 |
| MAX | chr14 | − | 65077912 | 65078036 | 65006173 | 65006284 |
| DNM1 | chr9 | + | 128253100 | 128253137 | 128250724 | 128250940 |
| SH3KBP1 | chrX | − | 19545921 | 19546050 | 19541924 | 19542193 |
| CADM1 | chr11 | − | 115209573 | 115209657 | 115178643 | 115178775 |
| RNPS1 | chr16 | − | 2264572 | 2264760 | 2264175 | 2264331 |
| DBN1 | chr5 | − | 177459602 | 177459740 | 177459097 | 177459268 |
| DLG1 | chr3 | − | 197161639 | 197161738 | 197149742 | 197149796 |
| PPP2R5B | chr11 | + | 64928065 | 64928158 | 64927801 | 64927903 |
| IFT46 | chr11 | − | 118557737 | 118557890 | 118556905 | 118557045 |
| FHL1 | chrX | + | 136209242 | 136209442 | 136208454 | 136208641 |
| ATXN2L | chr16 | + | 28836325 | 28836490 | 28835932 | 28836122 |
| COG1 | chr17 | + | 73207701 | 73207739 | 73207180 | 73207256 |
| SNHG14 | chr15 | + | 25391595 | 25391791 | 25278846 | 25278884 |
| MPRIP | chr17 | + | 17175292 | 17175412 | 17173915 | 17174075 |
| B4GALNT1 | chr12 | − | 57631199 | 57631364 | 57630979 | 57631086 |
| USP16 | chr21 | + | 29025708 | 29025771 | 29024650 | 29024777 |
| UNC13B | chr9 | + | 35401950 | 35402007 | 35400295 | 35400443 |
| SORBS1 | chr10 | − | 95355626 | 95357105 | 95354881 | 95354967 |
| SORBS1 | chr10 | − | 95371325 | 95371427 | 95367649 | 95367699 |
| SORBS1 | chr10 | − | 95422189 | 95422216 | 95421960 | 95422073 |
| SIPA1L2 | chr1 | − | 232404124 | 232404178 | 232403447 | 232403571 |
| PRDM2 | chr1 | + | 13773077 | 13773188 | 13749360 | 13749487 |
| ISOC2 | chr19 | − | 55455635 | 55455845 | 55455259 | 55455330 |
| FGFR2 | chr10 | − | 121564501 | 121564579 | 121551289 | 121551459 |
| EPB41L2 | chr6 | − | 130869811 | 130870126 | 130867458 | 130867581 |
| EPB41L2 | chr6 | − | 130870327 | 130870381 | 130867458 | 130867581 |
| MTA1 | chr14 | + | 105468337 | 105468349 | 105466706 | 105466742 |
| CHURC1 | chr14 | + | 64926009 | 64926080 | 64923990 | 64924126 |
| PLS3 | chrX | + | 115640109 | 115640136 | 115636835 | 115636978 |
| NDRG2 | chr14 | − | 21022863 | 21022905 | 21022391 | 21022497 |
| PNPLA8 | chr7 | − | 108521475 | 108521521 | 108514435 | 108515574 |
| FEZ2 | chr2 | − | 36560784 | 36560865 | 36558437 | 36558513 |
| AKIP1 | chr11 | + | 8912452 | 8912533 | 8911191 | 8911671 |
| RNF34 | chr12 | + | 121402741 | 121402807 | 121400118 | 121400218 |
| LLGL1 | chr17 | + | 18242742 | 18242822 | 18242507 | 18242628 |
| LUC7L | chr16 | − | 228332 | 228402 | 227241 | 227336 |
| PTP4A2 | chr1 | − | 31915894 | 31915987 | 31911695 | 31911826 |
| LMO3 | chr12 | − | 16606065 | 16606132 | 16600654 | 16600868 |
| EIF4A2 | chr3 | + | 186784961 | 186785101 | 186784563 | 186784696 |
| KIAA1191 | chr5 | − | 176359810 | 176359918 | 176355570 | 176355749 |
| CHD3 | chr17 | + | 7907600 | 7907702 | 7907352 | 7907488 |
| GPM6B | chrX | − | 13807649 | 13807769 | 13785621 | 13785808 |
| UPP1 | chr7 | + | 48101823 | 48101982 | 48094762 | 48094827 |
| MROH1 | chr8 | + | 144242601 | 144242628 | 144242368 | 144242515 |
| RBM4 | chr11 | + | 66643449 | 66644140 | 66638934 | 66640123 |
| IPO13 | chr1 | + | 43964268 | 43964321 | 43961165 | 43961262 |
| FAM204A | chr10 | − | 118341726 | 118341927 | 118336181 | 118336423 |
| CCDC92 | chr12 | − | 123944271 | 123944364 | 123943346 | 123943493 |
| ATP5SL | chr19 | − | 41433271 | 41433434 | 41431318 | 41432408 |
| FAM65A | chr16 | + | 67529636 | 67529859 | 67528880 | 67528914 |
| SS18 | chr18 | − | 26035830 | 26035923 | 26035004 | 26035127 |
| PXK | chr3 | + | 58423460 | 58423494 | 58420535 | 58420568 |
| POLB | chr8 | + | 42339011 | 42339069 | 42338454 | 42338685 |
| MRPL43 | chr10 | − | 100987089 | 100987196 | 100986275 | 100986975 |
| TMEM63B | chr6 | + | 44135327 | 44135366 | 44135016 | 44135096 |
| AXIN1 | chr16 | − | 291189 | 291297 | 289439 | 289607 |
| NME4 | chr16 | + | 398207 | 398394 | 397191 | 397313 |
| CLTA | chr9 | + | 36197550 | 36197588 | 36190855 | 36191273 |
| CLTA | chr9 | + | 36198978 | 36199096 | 36190855 | 36191273 |
| CLTA | chr9 | + | 36209266 | 36209320 | 36204067 | 36204179 |
| GRIA2 | chr4 | + | 157361537 | 157361652 | 157361009 | 157361124 |
| ACAA1 | chr3 | − | 38126161 | 38126341 | 38125825 | 38125881 |
| MBD1 | chr18 | − | 50273333 | 50273471 | 50272823 | 50272955 |
| EAPP | chr14 | − | 34529357 | 34529475 | 34524696 | 34524807 |
| MYO18A | chr17 | − | 29085603 | 29085648 | 29082315 | 29082438 |
| PKIG | chr20 | + | 44582584 | 44582731 | 44531784 | 44531978 |
| KCNQ2 | chr20 | − | 63411774 | 63411882 | 63408412 | 63408536 |
| PAM | chr5 | + | 103025130 | 103025334 | 103019789 | 103019843 |
| NRCAM | chr7 | − | 108166920 | 108167073 | 108160360 | 108160492 |
| NRCAM | chr7 | − | 108175321 | 108175357 | 108160360 | 108160492 |
| NRCAM | chr7 | − | 108191253 | 108191283 | 108189644 | 108189746 |
| NRCAM | chr7 | − | 108237751 | 108237769 | 108234582 | 108234688 |
| SMIM8 | chr6 | + | 87330691 | 87330712 | 87322587 | 87322632 |
| SMUG1 | chr12 | − | 54187818 | 54187905 | 54183655 | 54183959 |
| FBXL6 | chr8 | − | 144357438 | 144357502 | 144356989 | 144357121 |
| GIPC1 | chr19 | − | 14492856 | 14492912 | 14491655 | 14491743 |
| CHD5 | chr1 | − | 6106394 | 6106509 | 6106233 | 6106287 |
| RBCK1 | chr20 | + | 420870 | 421031 | 419557 | 419731 |
| CADPS2 | chr7 | − | 122414067 | 122414076 | 122407539 | 122407696 |
| TRIM46 | chr1 | + | 155178808 | 155178846 | 155178491 | 155178613 |
| SNHG5 | chr6 | − | 85677790 | 85677875 | 85677423 | 85677492 |
| SNHG5 | chr6 | − | 85677794 | 85677868 | 85677423 | 85677492 |
| SNHG5 | chr6 | − | 85677794 | 85677875 | 85677423 | 85677492 |
| SNHG5 | chr6 | − | 85677794 | 85677899 | 85677423 | 85677492 |
| CAST | chr5 | + | 96726753 | 96726859 | 96722638 | 96722698 |
| CAST | chr5 | + | 96726793 | 96726859 | 96722638 | 96722698 |
| CAST | chr5 | + | 96729152 | 96729209 | 96727488 | 96727530 |
| MEAF6 | chr1 | − | 37496706 | 37496736 | 37495884 | 37495918 |
| KIF3A | chr5 | − | 132706450 | 132706459 | 132703462 | 132703619 |
| KIF3A | chr5 | − | 132708906 | 132708978 | 132703462 | 132703619 |
| CCDC85A | chr2 | + | 56372343 | 56372478 | 56342878 | 56342955 |
| SRCAP | chr16 | + | 30722562 | 30722748 | 30722121 | 30722286 |
| ACD | chr16 | − | 67658554 | 67658641 | 67657985 | 67658362 |
| UPF3A | chr13 | + | 114286301 | 114286400 | 114282836 | 114282943 |
| ATP2B2 | chr3 | − | 10379242 | 10379284 | 10378251 | 10378410 |
| DCTN1 | chr2 | − | 74363613 | 74363628 | 74363293 | 74363427 |
| PLA2G6 | chr22 | − | 38128268 | 38128430 | 38126370 | 38126449 |
| CCDC173 | chr2 | − | 169650328 | 169650521 | 169649181 | 169649346 |
| ZFYVE27 | chr10 | + | 97752856 | 97752877 | 97751375 | 97751462 |
| TTLL3 | chr3 | + | 9833103 | 9833245 | 9828959 | 9829395 |
| DENND3 | chr8 | + | 141190283 | 141190417 | 141188348 | 141189146 |
| MFF | chr2 | + | 227347225 | 227347384 | 227340291 | 227340380 |
| MCCC1 | chr3 | − | 183092408 | 183092545 | 183086692 | 183086788 |
| MBNL2 | chr13 | + | 97365135 | 97365171 | 97357481 | 97357635 |
| SUPT5H | chr19 | + | 39458293 | 39458305 | 39457674 | 39457740 |
| HNRNPM | chr19 | + | 8473663 | 8473708 | 8471325 | 8471427 |
| MEGF8 | chr19 | + | 42371349 | 42371482 | 42370700 | 42370831 |
| IQCB1 | chr3 | − | 121788283 | 121788432 | 121781742 | 121781874 |
| LRRFIP1 | chr2 | + | 237727875 | 237727935 | 237720771 | 237720822 |
| ERGIC3 | chr20 | + | 35554371 | 35554386 | 35548807 | 35548865 |
| ZFAS1 | chr20 | + | 49280902 | 49280964 | 49280484 | 49280570 |
| PQBP1 | chrX | + | 48902232 | 48902517 | 48901929 | 48902042 |
| EIF4G1 | chr3 | + | 184315488 | 184315545 | 184314494 | 184314674 |
| EIF4G1 | chr3 | + | 184316712 | 184316733 | 184316131 | 184316218 |
| VDAC3 | chr8 | + | 42396677 | 42396680 | 42395083 | 42395133 |
| MEG3 | chr14 | + | 100832511 | 100832641 | 100831420 | 100831534 |
| APLP2 | chr11 | + | 130137255 | 130137291 | 130135562 | 130135715 |
| ATG4D | chr19 | + | 10546838 | 10547115 | 10544956 | 10545130 |
| ANAPC11 | chr17 | + | 81893551 | 81893614 | 81891740 | 81891863 |
| SIN3B | chr19 | + | 16862883 | 16862979 | 16862351 | 16862559 |
| FN1 | chr2 | − | 215380810 | 215381080 | 215379129 | 215379317 |
| HM13 | chr20 | + | 31568077 | 31568224 | 31566209 | 31566295 |
| HM13 | chr20 | + | 31568077 | 31568280 | 31566209 | 31566295 |
| ICA1 | chr7 | − | 8141968 | 8142055 | 8141764 | 8141817 |
| SLMAP | chr3 | + | 57925844 | 57925934 | 57922888 | 57923023 |
| RAB11FIP5 | chr2 | − | 73079650 | 73081663 | 73075992 | 73076182 |
| RP5-1022I14.1 | chr7 | − | 43969731 | 43969861 | 43966263 | 43966395 |
| CLTB | chr5 | − | 176396478 | 176396532 | 176392454 | 176392945 |
| RNF130 | chr5 | − | 179963470 | 179963564 | 179955066 | 179955669 |
| CAMLG | chr5 | + | 134741062 | 134741523 | 134738500 | 134738792 |
| CAMLG | chr5 | + | 134741062 | 134741523 | 134738500 | 134738792 |
| PDE4DIP | chr1 | + | 149012590 | 149012776 | 149010442 | 149010595 |
| DTNA | chr18 | + | 34829399 | 34829489 | 34827592 | 34827676 |
| CD151 | chr11 | + | 834529 | 834591 | 832842 | 833026 |
| GRAMD1A | chr19 | + | 35022899 | 35022911 | 35021950 | 35022038 |
| ING4 | chr12 | − | 6653229 | 6653396 | 6652935 | 6653050 |
| MAN2C1 | chr15 | − | 75363998 | 75364188 | 75362641 | 75362748 |
| FXYD6 | chr11 | − | 117844109 | 117844206 | 117842718 | 117842781 |
| DCTN2 | chr12 | − | 57538518 | 57538524 | 57535748 | 57535845 |
| MPPE1 | chr18 | − | 11906202 | 11906309 | 11896983 | 11897356 |
| TPM2 | chr9 | − | 35684731 | 35684807 | 35684487 | 35684550 |
| TPM2 | chr9 | − | 35685063 | 35685139 | 35684731 | 35684807 |

| | downstream | downstream | | | | |
|---|---|---|---|---|---|---|
| geneSymbol | ES | EE | ID. 1 | PValue | FDR | type |
| EXOC7 | 76087653 | 76087720 | 69 | 1.698E−05 | 2.034E−03 | UP |
| EXOC7 | 76094413 | 76094581 | 74 | 9.606E−09 | 7.564E−06 | UP |
| GRIPAP1 | 48982978 | 48983092 | 98 | 3.196E−06 | 6.292E−04 | UP |
| EPB41L3 | 5410565 | 5410619 | 313 | 5.077E−04 | 2.103E−02 | UP |
| BANF1 | 66003625 | 66003898 | 329 | 2.123E−08 | 1.373E−05 | UP |
| JOSD2 | 50510285 | 50510445 | 780 | 2.685E−06 | 5.658E−04 | UP |
| SLC25A26 | 66243202 | 66243312 | 1213 | 1.160E−05 | 1.522E−03 | UP |
| ARHGAP23 | 38500596 | 38500628 | 1912 | 2.294E−04 | 1.255E−02 | UP |
| CLIP2 | 74375886 | 74376822 | 1935 | 2.967E−06 | 6.102E−04 | UP |
| SH3BP5 | 15258830 | 15259050 | 2204 | 1.203E−03 | 3.690E−02 | DN |
| IL17RC | 9932952 | 9933617 | 2297 | 1.823E−04 | 1.055E−02 | UP |
| EEF1D | 143597347 | 143597415 | 2382 | 2.547E−05 | 2.697E−03 | UP |
| EEF1D | 143597347 | 143597675 | 2383 | 4.260E−05 | 3.886E−03 | UP |
| SAT2 | 7627142 | 7627226 | 2466 | 2.816E−07 | 1.074E−04 | UP |
| RGS14 | 177371872 | 177372601 | 2676 | 1.670E−03 | 4.559E−02 | UP |
| RNF14 | 141980122 | 141980351 | 3295 | 4.443E−04 | 1.932E−02 | UP |
| CMC2 | 81006733 | 81006858 | 3443 | 2.039E−05 | 2.294E−03 | UP |
| BCAS4 | 50876485 | 50876723 | 3713 | 1.514E−03 | 4.261E−02 | DN |
| C1orf61 | 156414653 | 156414753 | 3796 | 2.333E−05 | 2.538E−03 | UP |
| KIF21A | 39351776 | 39351980 | 3841 | 8.075E−04 | 2.851E−02 | UP |
| LRRFIP2 | 37075023 | 37075116 | 4177 | 1.724E−05 | 2.034E−03 | UP |
| LRRFIP2 | 37075023 | 37075116 | 4178 | 1.944E−04 | 1.110E−02 | UP |
| CABP1 | 120659877 | 120659908 | 4465 | 1.319E−03 | 3.885E−02 | UP |
| DNM2 | 10812263 | 10812377 | 4590 | 5.423E−04 | 2.185E−02 | DN |
| ARHGEF1 | 41906702 | 41906803 | 4605 | 5.109E−06 | 8.939E−04 | UP |
| ABTB1 | 127677167 | 127677286 | 4651 | 3.107E−04 | 1.542E−02 | UP |
| PLXNB3 | 153778395 | 153778471 | 4712 | 6.872E−09 | 5.657E−06 | UP |
| ACTR2 | 65246523 | 65246739 | 4965 | 8.153E−06 | 1.210E−03 | UP |
| ATP6V0A1 | 42513860 | 42513978 | 5185 | 1.108E−03 | 3.524E−02 | UP |
| APMAP | 24969525 | 24969660 | 5220 | 1.137E−06 | 3.007E−04 | UP |
| CHGA | 92932369 | 92932851 | 5378 | 9.506E−07 | 2.755E−04 | UP |
| ZFAND5 | 72364695 | 72365197 | 6322 | 2.599E−04 | 1.366E−02 | UP |
| RPS24 | 78040203 | 78040225 | 6564 | 2.977E−12 | 8.987E−09 | UP |
| RPS24 | 78040203 | 78040225 | 6565 | 4.052E−05 | 3.756E−03 | UP |
| RALYL | 84849979 | 84850027 | 6958 | 4.174E−04 | 1.862E−02 | UP |
| TXNL4A | 79988239 | 79988593 | 7159 | 9.027E−05 | 6.605E−03 | DN |
| SNAP25 | 10293160 | 10293278 | 7248 | 0.000E+00 | 0.000E+00 | DN |
| SNAP25 | 10296924 | 10297050 | 7252 | 6.259E−13 | 2.267E−09 | UP |
| LUC7L2 | 139376061 | 139376156 | 7708 | 1.631E−03 | 4.485E−02 | DN |
| TPM1 | 63062214 | 63062277 | 7832 | 7.985E−04 | 2.825E−02 | UP |
| MTMR3 | 30025629 | 30026037 | 8070 | 1.490E−03 | 4.212E−02 | DN |
| BID | 17774380 | 17774412 | 8209 | 1.604E−04 | 9.849E−03 | UP |
| TPD52L2 | 63889189 | 63889238 | 8293 | 1.147E−03 | 3.600E−02 | UP |
| MVK | 109586021 | 109586125 | 8372 | 7.621E−04 | 2.752E−02 | UP |
| PLEKHJ1 | 2235761 | 2235828 | 8537 | 2.696E−05 | 2.797E−03 | UP |
| TM7SF2 | 65115475 | 65115598 | 8939 | 7.761E−04 | 2.778E−02 | UP |
| PTK2 | 140674297 | 140674404 | 9059 | 9.996E−10 | 1.322E−06 | UP |
| ZFYVE19 | 40813337 | 40813417 | 10417 | 1.824E−03 | 4.856E−02 | DN |
| SRM | 11056010 | 11056094 | 10655 | 3.405E−06 | 6.631E−04 | UP |
| PPP5C | 46383779 | 46383878 | 10866 | 9.751E−04 | 3.211E−02 | UP |
| AP2B1 | 35671753 | 35671900 | 10897 | 1.301E−05 | 1.654E−03 | UP |
| RBM42 | 35633097 | 35633252 | 10953 | 4.805E−04 | 2.040E−02 | DN |
| TTYH1 | 54436090 | 54436171 | 11988 | 1.236E−03 | 3.753E−02 | DN |
| MARK4 | 45299810 | 45299855 | 12041 | 3.350E−04 | 1.618E−02 | UP |
| MAX | 65093707 | 65093815 | 12160 | 9.279E−04 | 3.123E−02 | UP |
| DNM1 | 128254653 | 128255244 | 12180 | 1.561E−03 | 4.330E−02 | UP |
| SH3KBP1 | 19549973 | 19550083 | 12188 | 6.979E−04 | 2.598E−02 | DN |
| CADM1 | 115214607 | 115214780 | 12346 | 1.553E−03 | 4.320E−02 | DN |
| RNPS1 | 2268054 | 2268095 | 12502 | 6.274E−05 | 5.084E−03 | UP |
| DBN1 | 177460431 | 177460555 | 12706 | 1.234E−05 | 1.585E−03 | UP |
| DLG1 | 197194424 | 197194589 | 13008 | 1.862E−03 | 4.911E−02 | DN |
| PPP2R5B | 64928294 | 64928425 | 13788 | 1.594E−03 | 4.398E−02 | UP |
| IFT46 | 118559784 | 118559864 | 13863 | 3.222E−04 | 1.577E−02 | UP |
| FHL1 | 136209870 | 136211359 | 14591 | 8.364E−07 | 2.483E−04 | UP |
| ATXN2L | 28836727 | 28837237 | 15093 | 6.540E−04 | 2.496E−02 | UP |
| COG1 | 73208313 | 73208503 | 15358 | 9.982E−05 | 7.076E−03 | DN |
| SNHG14 | 25418787 | 25419462 | 15399 | 5.709E−05 | 4.754E−03 | UP |
| MPRIP | 17176425 | 17176512 | 15408 | 2.078E−09 | 2.427E−06 | UP |
| B4GALNT1 | 57631914 | 57632133 | 15535 | 4.503E−04 | 1.951E−02 | UP |
| USP16 | 29027872 | 29027974 | 15901 | 1.159E−04 | 7.838E−03 | UP |
| UNC13B | 35403166 | 35403259 | 15975 | 8.931E−04 | 3.052E−02 | UP |
| SORBS1 | 95357630 | 95357803 | 16125 | 1.942E−04 | 1.110E−02 | UP |
| SORBS1 | 95375972 | 95376056 | 16127 | 6.261E−04 | 2.420E−02 | UP |
| SORBS1 | 95432447 | 95432576 | 16141 | 1.369E−03 | 3.993E−02 | UP |
| SIPA1L2 | 232415493 | 232415625 | 16440 | 3.452E−05 | 3.370E−03 | UP |
| PRDM2 | 13816426 | 13816570 | 16744 | 3.749E−04 | 1.741E−02 | DN |
| ISOC2 | 55456348 | 55456489 | 16750 | 1.110E−16 | 2.011E−12 | DN |
| FGFR2 | 121593708 | 121593967 | 16764 | 1.043E−05 | 1.432E−03 | DN |
| EPB41L2 | 130885095 | 130885268 | 16781 | 3.286E−04 | 1.596E−02 | UP |
| EPB41L2 | 130885095 | 130885268 | 16782 | 5.351E−04 | 2.162E−02 | UP |
| MTA1 | 105469466 | 105469498 | 16992 | 4.038E−04 | 1.821E−02 | UP |
| CHURC1 | 64932137 | 64934385 | 17142 | 7.113E−04 | 2.634E−02 | UP |
| PLS3 | 115640407 | 115640503 | 17192 | 7.881E−05 | 5.984E−03 | UP |
| NDRG2 | 21023240 | 21023321 | 17267 | 9.619E−04 | 3.185E−02 | UP |
| PNPLA8 | 108526028 | 108526194 | 17384 | 1.867E−03 | 4.911E−02 | DN |
| FEZ2 | 36578596 | 36578865 | 17968 | 2.992E−04 | 1.505E−02 | UP |
| AKIP1 | 8914825 | 8914930 | 18082 | 3.794E−05 | 3.588E−03 | DN |
| RNF34 | 121416158 | 121416377 | 18598 | 1.628E−06 | 3.855E−04 | DN |
| LLGL1 | 18243907 | 18244875 | 18735 | 1.373E−03 | 3.998E−02 | UP |
| LUC7L | 229278 | 229450 | 18752 | 2.642E−05 | 2.758E−03 | DN |
| PTP4A2 | 31918969 | 31919109 | 19372 | 2.858E−04 | 1.452E−02 | DN |
| LMO3 | 16609733 | 16609889 | 19379 | 2.516E−05 | 2.673E−03 | UP |
| EIF4A2 | 186785882 | 186786051 | 19944 | 3.363E−11 | 8.700E−08 | UP |
| KIAA1191 | 176361601 | 176361764 | 20573 | 2.211E−05 | 2.449E−03 | DN |
| CHD3 | 7907893 | 7908019 | 20695 | 4.915E−04 | 2.063E−02 | UP |
| GPM6B | 13938506 | 13938638 | 20757 | 1.131E−06 | 3.007E−04 | UP |
| UPP1 | 48106872 | 48107082 | 21016 | 1.640E−03 | 4.503E−02 | UP |
| MROH1 | 144243493 | 144243616 | 21572 | 8.790E−04 | 3.026E−02 | DN |
| RBM4 | 66646026 | 66646452 | 21619 | 7.267E−06 | 1.120E−03 | UP |
| IPO13 | 43966574 | 43966641 | 21660 | 6.167E−04 | 2.406E−02 | UP |
| FAM204A | 118342269 | 118342320 | 21892 | 4.212E−06 | 7.744E−04 | UP |
| CCDC92 | 123972528 | 123972824 | 21934 | 3.857E−04 | 1.773E−02 | UP |
| ATP5SL | 41433536 | 41433673 | 22186 | 2.282E−05 | 2.513E−03 | UP |
| FAM65A | 67538423 | 67538550 | 22376 | 8.924E−04 | 3.052E−02 | DN |
| SS18 | 26038554 | 26038659 | 22457 | 3.232E−04 | 1.578E−02 | UP |
| PXK | 58424751 | 58426019 | 22805 | 3.607E−07 | 1.293E−04 | DN |
| POLB | 42344952 | 42345019 | 22930 | 5.384E−05 | 4.556E−03 | DN |
| MRPL43 | 100987312 | 100987456 | 22951 | 4.377E−05 | 3.954E−03 | UP |
| TMEM63B | 44136348 | 44136439 | 23158 | 1.246E−03 | 3.775E−02 | DN |
| AXIN1 | 293487 | 293718 | 24004 | 9.368E−04 | 3.133E−02 | UP |
| NME4 | 398989 | 399123 | 24027 | 1.140E−03 | 3.588E−02 | UP |
| CLTA | 36204067 | 36204179 | 24092 | 1.215E−05 | 1.566E−03 | DN |
| CLTA | 36204067 | 36204179 | 24093 | 1.176E−06 | 3.064E−04 | DN |
| CLTA | 36211602 | 36212059 | 24099 | 2.588E−04 | 1.362E−02 | UP |
| GRIA2 | 157362798 | 157363047 | 24169 | 1.120E−03 | 3.555E−02 | DN |
| ACAA1 | 38126509 | 38126700 | 24707 | 1.636E−04 | 9.974E−03 | UP |
| MBD1 | 50273563 | 50273863 | 25291 | 3.542E−05 | 3.439E−03 | UP |
| EAPP | 34533443 | 34533539 | 25334 | 1.458E−04 | 9.123E−03 | DN |
| MYO18A | 29086437 | 29086577 | 25680 | 4.803E−11 | 1.025E−07 | DN |
| PKIG | 44589796 | 44589866 | 25690 | 1.236E−06 | 3.154E−04 | DN |
| KCNQ2 | 63413449 | 63413581 | 27130 | 3.909E−04 | 1.783E−02 | UP |
| PAM | 103028181 | 103028238 | 27390 | 1.628E−04 | 9.962E−03 | UP |
| NRCAM | 108176429 | 108176606 | 27645 | 1.141E−04 | 7.754E−03 | UP |
| NRCAM | 108176429 | 108176606 | 27650 | 2.826E−09 | 2.902E−06 | UP |
| NRCAM | 108191728 | 108191853 | 27653 | 1.442E−06 | 3.506E−04 | UP |
| NRCAM | 108239958 | 108240049 | 27655 | 3.018E−05 | 3.060E−03 | UP |
| SMIM8 | 87337008 | 87337166 | 27925 | 2.683E−06 | 5.658E−04 | UP |
| SMUG1 | 54188950 | 54188974 | 28582 | 1.571E−04 | 9.664E−03 | UP |
| FBXL6 | 144357627 | 144357786 | 28646 | 1.255E−03 | 3.782E−02 | UP |
| GIPC1 | 14496036 | 14496132 | 28780 | 9.674E−04 | 3.191E−02 | DN |
| CHD5 | 6106615 | 6106779 | 28894 | 1.473E−03 | 4.184E−02 | UP |
| RBCK1 | 422126 | 422238 | 29193 | 3.417E−04 | 1.639E−02 | DN |
| CADPS2 | 122416060 | 122416164 | 30281 | 1.813E−03 | 4.840E−02 | UP |
| TRIM46 | 155179631 | 155179935 | 30914 | 9.287E−04 | 3.123E−02 | DN |
| SNHG5 | 85677953 | 85678032 | 31133 | 3.910E−07 | 1.362E−04 | DN |
| SNHG5 | 85677953 | 85678032 | 31134 | 2.810E−06 | 5.815E−04 | DN |
| SNHG5 | 85677953 | 85678032 | 31135 | 8.372E−08 | 4.395E−05 | DN |
| SNHG5 | 85677953 | 85678032 | 31136 | 7.156E−07 | 2.215E−04 | DN |
| CAST | 96727488 | 96727530 | 31169 | 9.389E−05 | 6.815E−03 | DN |
| CAST | 96727488 | 96727530 | 31170 | 1.429E−07 | 6.634E−05 | DN |
| CAST | 96729611 | 96729725 | 31171 | 3.298E−04 | 1.599E−02 | DN |
| MEAF6 | 37501803 | 37501996 | 31266 | 2.998E−04 | 1.506E−02 | UP |
| KIF3A | 132710958 | 132711057 | 31429 | 4.245E−05 | 3.883E−03 | UP |
| KIF3A | 132710958 | 132711057 | 31430 | 1.520E−04 | 9.429E−03 | UP |
| CCDC85A | 56375815 | 56375935 | 31483 | 1.198E−05 | 1.550E−03 | UP |
| SRCAP | 30722962 | 30723229 | 31490 | 4.684E−04 | 2.008E−02 | UP |
| ACD | 67658719 | 67658816 | 31626 | 4.124E−04 | 1.846E−02 | UP |
| UPF3A | 114286518 | 114286629 | 32026 | 8.858E−04 | 3.043E−02 | UP |
| ATP2B2 | 10388276 | 10388402 | 32126 | 6.789E−08 | 3.616E−05 | DN |
| DCTN1 | 74365074 | 74365241 | 33128 | 2.628E−06 | 5.658E−04 | UP |
| PLA2G6 | 38129453 | 38129562 | 33256 | 1.246E−06 | 3.154E−04 | DN |
| CCDC173 | 169654074 | 169654214 | 33287 | 1.276E−03 | 3.807E−02 | UP |
| ZFYVE27 | 97753037 | 97753182 | 33312 | 1.425E−06 | 3.488E−04 | UP |
| TTLL3 | 9834680 | 9834907 | 33754 | 1.144E−03 | 3.593E−02 | DN |
| DENND3 | 141192330 | 141192449 | 33816 | 1.191E−05 | 1.546E−03 | DN |
| MFF | 227355676 | 227355761 | 33955 | 1.731E−04 | 1.020E−02 | UP |
| MCCC1 | 183094558 | 183094605 | 34188 | 1.689E−03 | 4.594E−02 | UP |
| MBNL2 | 97391321 | 97394119 | 34284 | 1.224E−03 | 3.730E−02 | UP |
| SUPT5H | 39458817 | 39458887 | 34901 | 2.096E−07 | 8.931E−05 | DN |
| HNRNPM | 8474166 | 8474244 | 35034 | 9.491E−05 | 6.870E−03 | DN |
| MEGF8 | 42375506 | 42377786 | 35227 | 3.395E−04 | 1.633E−02 | UP |
| IQCB1 | 121790072 | 121790215 | 35325 | 1.386E−03 | 4.017E−02 | UP |
| LRRFIP1 | 237739231 | 237739309 | 35596 | 3.756E−05 | 3.562E−03 | UP |
| ERGIC3 | 35555043 | 35555075 | 36222 | 1.411E−03 | 4.048E−02 | UP |
| ZFAS1 | 49289044 | 49289260 | 36804 | 1.356E−04 | 8.737E−03 | UP |
| PQBP1 | 48902731 | 48902795 | 36886 | 1.046E−08 | 7.893E−06 | UP |
| EIF4G1 | 184315762 | 184315856 | 36974 | 1.967E−04 | 1.118E−02 | UP |
| EIF4G1 | 184317320 | 184317497 | 36980 | 5.924E−05 | 4.866E−03 | UP |
| VDAC3 | 42398711 | 42398864 | 38160 | 3.161E−04 | 1.558E−02 | UP |
| MEG3 | 100835738 | 100835879 | 38245 | 2.664E−04 | 1.380E−02 | DN |
| APLP2 | 130140397 | 130140483 | 38692 | 3.641E−09 | 3.381E−06 | UP |
| ATG4D | 10547188 | 10547253 | 38776 | 3.277E−07 | 1.199E−04 | UP |
| ANAPC11 | 81894466 | 81894586 | 39280 | 7.119E−04 | 2.634E−02 | UP |
| SIN3B | 16863679 | 16863796 | 39611 | 7.302E−06 | 1.120E−03 | DN |
| FN1 | 215382211 | 215382325 | 39947 | 3.472E−04 | 1.655E−02 | DN |
| HM13 | 31569119 | 31571606 | 40204 | 1.657E−03 | 4.531E−02 | UP |
| HM13 | 31569119 | 31569567 | 40206 | 4.729E−04 | 2.022E−02 | UP |
| ICA1 | 8143874 | 8143972 | 40260 | 1.085E−04 | 7.484E−03 | DN |
| SLMAP | 57927295 | 57927486 | 40276 | 9.663E−04 | 3.191E−02 | DN |
| RAB11FIP5 | 73088049 | 73088749 | 40792 | 3.621E−04 | 1.701E−02 | UP |
| RP5-1022I14.1 | 43972629 | 43972691 | 41025 | 5.052E−07 | 1.637E−04 | UP |
| CLTB | 176397606 | 176397718 | 41052 | 1.642E−08 | 1.122E−05 | UP |
| RNF130 | 179966805 | 179967010 | 41279 | 3.222E−04 | 1.577E−02 | UP |
| CAMLG | 134743986 | 134744052 | 41480 | 1.854E−14 | 9.881E−11 | UP |
| CAMLG | 134750758 | 134752160 | 41481 | 7.522E−07 | 2.258E−04 | UP |
| PDE4DIP | 149016298 | 149016550 | 41546 | 9.517E−04 | 3.169E−02 | UP |
| DTNA | 34848295 | 34848383 | 41599 | 2.905E−05 | 2.970E−03 | DN |
| CD151 | 836062 | 836153 | 41669 | 1.462E−03 | 4.162E−02 | DN |
| GRAMD1A | 35023235 | 35023343 | 41887 | 4.331E−04 | 1.902E−02 | UP |
| ING4 | 6656726 | 6656798 | 42021 | 1.166E−03 | 3.638E−02 | UP |
| MAN2C1 | 75364487 | 75364665 | 42272 | 3.189E−04 | 1.565E−02 | UP |
| FXYD6 | 117877298 | 117877486 | 42898 | 2.207E−05 | 2.449E−03 | UP |
| DCTN2 | 57546027 | 57546096 | 42918 | 7.093E−07 | 2.215E−04 | UP |
| MPPE1 | 11908200 | 11908330 | 42947 | 5.612E−04 | 2.254E−02 | UP |
| TPM2 | 35685063 | 35685139 | 43450 | 1.840E−05 | 2.137E−03 | UP |
| TPM2 | 35685268 | 35685339 | 43451 | 7.408E−04 | 2.705E−02 | DN |

TABLE 26
FXS_VS_TD_MXE

| | | | 1stExon | 1stExon | 2ndExon | 2ndExon | upstream |
|---|---|---|---|---|---|---|---|
| geneSymbol | chr | strand | Start_0base | End | Start_0base | End | ES |
| NEGR1 | chr1 | − | 71698007 | 71698139 | 71776171 | 71776297 | 71611025 |
| RPL9 | chr4 | − | 39456405 | 39456538 | 39457585 | 39457681 | 39454863 |
| C17orf101 | chr17 | − | 82406417 | 82406482 | 82415397 | 82415627 | 82405323 |
| ARHGAP23 | chr17 | + | 38498413 | 38498510 | 38498931 | 38498954 | 38497784 |
| DUSP22 | chr6 | + | 311879 | 311962 | 335113 | 335163 | 304627 |
| ZCCHC17 | chr1 | + | 31346639 | 31346740 | 31348828 | 31348974 | 31338956 |
| ADHFE1 | chr8 | + | 66440161 | 66440199 | 66444366 | 66444420 | 66432485 |
| FHOD3 | chr18 | + | 36709094 | 36709391 | 36717831 | 36718715 | 36693208 |
| C1orf61 | chr1 | − | 156407841 | 156407975 | 156426797 | 156426872 | 156407079 |
| C1orf61 | chr1 | − | 156414653 | 156414753 | 156426797 | 156426872 | 156407841 |
| COMMD4 | chr15 | + | 75338061 | 75338133 | 75338354 | 75338420 | 75336098 |
| ANKS3 | chr16 | − | 4724749 | 4724831 | 4729979 | 4730151 | 4714050 |
| C6orf136 | chr6 | + | 30649557 | 30649959 | 30651265 | 30651466 | 30647038 |
| C6orf136 | chr6 | + | 30649557 | 30649959 | 30651265 | 30651466 | 30647072 |
| C6orf136 | chr6 | + | 30651265 | 30651466 | 30652647 | 30652717 | 30650993 |
| SYT5 | chr19 | − | 55175708 | 55175876 | 55176004 | 55176124 | 55175171 |
| ATP2B1 | chr12 | − | 89634996 | 89635251 | 89642157 | 89642355 | 89634777 |
| RPS24 | chr10 | + | 78037438 | 78037441 | 78037964 | 78037982 | 78037193 |
| RALYL | chr8 | + | 84804769 | 84804802 | 84849979 | 84850027 | 84774578 |
| SNAP25 | chr20 | + | 10284723 | 10284772 | 10292881 | 10292999 | 10277684 |
| SNAP25 | chr20 | + | 10292881 | 10292999 | 10293160 | 10293278 | 10284723 |
| BFAR | chr16 | + | 14649803 | 14649973 | 14655065 | 14655210 | 14648387 |
| UPF3B | chrX | − | 119843190 | 119843301 | 119845197 | 119845296 | 119841734 |
| C12orf10 | chr12 | + | 53303033 | 53303193 | 53306197 | 53306320 | 53300149 |
| SCG5 | chr15 | + | 32679765 | 32679915 | 32691709 | 32691763 | 32643585 |
| N4BP2L2 | chr13 | − | 32521372 | 32521449 | 32527407 | 32527532 | 32510291 |
| SERPING1 | chr11 | + | 57602034 | 57602169 | 57606009 | 57606213 | 57598248 |
| WDR6 | chr3 | + | 49011634 | 49014116 | 49014209 | 49014293 | 49007061 |
| PDLIM2 | chr8 | + | 22585320 | 22585399 | 22589297 | 22589374 | 22585016 |
| PTK2 | chr8 | − | 140664916 | 140664997 | 140668268 | 140668424 | 140657899 |
| NPM2 | chr8 | + | 22034108 | 22034275 | 22034509 | 22034544 | 22033129 |
| ZNF75A | chr16 | + | 3313048 | 3313175 | 3316911 | 3317022 | 3312676 |
| RABEPK | chr9 | + | 125203007 | 125203066 | 125220538 | 125220700 | 125200602 |
| RABEPK | chr9 | + | 125207563 | 125207721 | 125220538 | 125220700 | 125203007 |
| DHRSX | chrX | − | 2291501 | 2291603 | 2408744 | 2408813 | 2266739 |
| ANAPC7 | chr12 | − | 110377392 | 110377617 | 110381751 | 110381948 | 110375476 |
| PUS1 | chr12 | + | 131939172 | 131939275 | 131941291 | 131941983 | 131931514 |
| FAM153B | chr5 | + | 176103755 | 176103780 | 176106058 | 176106129 | 176103226 |
| CSTF3 | chr11 | − | 33090531 | 33090727 | 33092270 | 33092340 | 33086987 |
| PHF20 | chr20 | + | 35842572 | 35842744 | 35858301 | 35858381 | 35801490 |
| APLP1 | chr19 | + | 35878084 | 35878108 | 35878583 | 35878654 | 35877717 |
| SIRPA | chr20 | + | 1921394 | 1921712 | 1924763 | 1924877 | 1915098 |
| MAP4 | chr3 | − | 47977864 | 47977933 | 47998637 | 47998879 | 47928227 |
| EDEM2 | chr20 | − | 35131641 | 35131783 | 35134737 | 35134949 | 35126250 |
| PPP2R2B | chr5 | − | 146701044 | 146701142 | 146878001 | 146878195 | 146697978 |
| ATP5B | chr12 | − | 56643836 | 56643958 | 56645170 | 56645353 | 56643402 |
| BIN1 | chr2 | − | 127051153 | 127051243 | 127057472 | 127057601 | 127050801 |
| BIN1 | chr2 | − | 127052254 | 127052362 | 127057472 | 127057601 | 127051153 |
| NUP205 | chr7 | + | 135630343 | 135630470 | 135635580 | 135635657 | 135627972 |
| DNAJB6 | chr7 | + | 157358546 | 157358637 | 157363160 | 157363270 | 157336965 |
| SYT1 | chr12 | + | 79217502 | 79217685 | 79285786 | 79285971 | 79047296 |
| METTL9 | chr16 | + | 21612644 | 21612835 | 21617864 | 21618074 | 21599475 |
| HDAC10 | chr22 | − | 50250062 | 50250160 | 50250426 | 50250523 | 50249859 |
| SIPA1L2 | chr1 | − | 232403447 | 232403571 | 232404124 | 232404178 | 232402391 |
| EPB41L2 | chr6 | − | 130865535 | 130865634 | 130869811 | 130870126 | 130863637 |
| C20orf27 | chr20 | − | 3758535 | 3758678 | 3760081 | 3760186 | 3755468 |
| EI24 | chr11 | + | 125578948 | 125579068 | 125580092 | 125580204 | 125578132 |
| EI24 | chr11 | + | 125580092 | 125580204 | 125581210 | 125581322 | 125578132 |
| UBXN4 | chr2 | + | 135772419 | 135772547 | 135776248 | 135776351 | 135770570 |
| ANKRD11 | chr16 | − | 89305205 | 89305344 | 89316932 | 89317078 | 89291012 |
| LUC7L | chr16 | − | 220648 | 220747 | 227241 | 227336 | 208077 |
| SNRPN | chr15 | + | 24967931 | 24968082 | 24976304 | 24976416 | 24962113 |
| SNRPN | chr15 | + | 24967931 | 24968082 | 24976876 | 24977029 | 24962113 |
| KIAA1191 | chr5 | − | 176350612 | 176350737 | 176359810 | 176359918 | 176348249 |
| KIAA1191 | chr5 | − | 176352621 | 176352748 | 176355570 | 176355749 | 176350612 |
| TBC1D7 | chr6 | − | 13306397 | 13306527 | 13307599 | 13307745 | 13304950 |
| ABHD12 | chr20 | − | 25302218 | 25302346 | 25303549 | 25303628 | 25294742 |
| LMF2 | chr22 | − | 50505056 | 50505153 | 50505228 | 50505334 | 50504801 |
| CLTA | chr9 | + | 36198978 | 36199096 | 36204067 | 36204179 | 36197550 |
| RALY | chr20 | + | 34077027 | 34077245 | 34078504 | 34078553 | 34076701 |
| ATP6V1H | chr8 | − | 53769617 | 53769743 | 53795646 | 53795839 | 53756554 |
| COX6C | chr8 | − | 99887489 | 99887618 | 99891907 | 99892052 | 99877865 |
| MBD1 | chr18 | − | 50273563 | 50273863 | 50274185 | 50274353 | 50272823 |
| C6orf1 | chr6 | − | 34247466 | 34247492 | 34247740 | 34247864 | 34247043 |
| RTN1 | chr14 | − | 59726918 | 59727668 | 59745707 | 59746481 | 59607284 |
| AKIRIN1 | chr1 | + | 38998170 | 38998311 | 39000971 | 39001106 | 38991287 |
| MPDZ | chr9 | − | 13113930 | 13114021 | 13115247 | 13115334 | 13113010 |
| MKKS | chr20 | − | 10412529 | 10413931 | 10420527 | 10420758 | 10408627 |
| ASPDH | chr19 | − | 50512135 | 50512290 | 50512359 | 50512580 | 50511599 |
| ASPDH | chr19 | − | 50512135 | 50512290 | 50512926 | 50513011 | 50511599 |
| MON1A | chr3 | − | 49909252 | 49909400 | 49910118 | 49910884 | 49907159 |
| MBP | chr18 | − | 76988494 | 76988527 | 76988876 | 76988912 | 76984774 |
| SULT1A1 | chr16 | − | 28608290 | 28608388 | 28608477 | 28608603 | 28606950 |
| PKM | chr15 | − | 72202453 | 72202620 | 72203021 | 72203188 | 72200473 |
| MOG | chr6 | + | 29659318 | 29659666 | 29666151 | 29666265 | 29657209 |
| RAB6A | chr11 | − | 73718612 | 73718718 | 73718784 | 73718890 | 73716250 |
| TMX2 | chr11 | + | 57737912 | 57738026 | 57738353 | 57738430 | 57737607 |
| PLEKHA6 | chr1 | − | 204223461 | 204223585 | 204228082 | 204228228 | 204218850 |
| TMEM185A | chrX | − | 149603986 | 149604070 | 149608626 | 149608834 | 149600302 |
| SNHG5 | chr6 | − | 85677423 | 85677492 | 85677953 | 85678032 | 85666003 |
| UPF3A | chr13 | + | 114282020 | 114282127 | 114282836 | 114282943 | 114281627 |
| UPF3A | chr13 | + | 114298839 | 114299000 | 114301730 | 114302025 | 114291633 |
| NDUFA8 | chr9 | − | 122148111 | 122148277 | 122152244 | 122152408 | 122144057 |
| EIF3L | chr22 | + | 37877673 | 37878171 | 37886764 | 37886845 | 37875840 |
| RIMS1 | chr6 | + | 72260704 | 72260767 | 72284046 | 72284118 | 72258985 |
| RIMS1 | chr6 | + | 72265959 | 72266049 | 72284046 | 72284118 | 72258985 |
| RIMS1 | chr6 | + | 72265959 | 72266049 | 72284046 | 72284118 | 72260704 |
| TSPAN3 | chr15 | − | 77055788 | 77055863 | 77056063 | 77056255 | 77054177 |
| WNK1 | chr12 | + | 900475 | 900670 | 904452 | 904494 | 897478 |
| LSM14B | chr20 | + | 62129784 | 62129952 | 62130218 | 62130296 | 62126303 |
| MCCC1 | chr3 | − | 183092408 | 183092545 | 183094558 | 183094605 | 183086692 |
| SPOCK3 | chr4 | − | 166754507 | 166754729 | 166792169 | 166792289 | 166741996 |
| IGSF8 | chr1 | − | 160092923 | 160093331 | 160094868 | 160095246 | 160092281 |
| SLC25A48 | chr5 | + | 135874020 | 135874154 | 135879967 | 135880097 | 135871460 |
| MDK | chr11 | + | 46382056 | 46382133 | 46382293 | 46382461 | 46380755 |
| HK1 | chr10 | + | 69382486 | 69382791 | 69398594 | 69398828 | 69379861 |
| TFPT | chr19 | − | 54110050 | 54110121 | 54114441 | 54114700 | 54108325 |
| GJB6 | chr13 | − | 20230697 | 20230807 | 20230865 | 20231061 | 20229579 |
| GJB6 | chr13 | − | 20230697 | 20230813 | 20230865 | 20231061 | 20229579 |
| MYT1L | chr2 | − | 1917204 | 1917339 | 1942981 | 1943334 | 1912019 |
| VIPR2 | chr7 | − | 159031937 | 159032067 | 159034212 | 159034304 | 159031827 |
| POLR2G | chr11 | + | 62761794 | 62761904 | 62762866 | 62763026 | 62761543 |
| VEZT | chr12 | + | 95282312 | 95282644 | 95287663 | 95287857 | 95274741 |
| CUX2 | chr12 | + | 111298540 | 111298589 | 111304209 | 111304314 | 111296472 |
| NR1H3 | chr11 | + | 47261546 | 47261726 | 47261918 | 47262018 | 47261240 |
| R3HDM2 | chr12 | − | 57310263 | 57310463 | 57395748 | 57395818 | 57303175 |
| ANAPC16 | chr10 | + | 72223887 | 72224056 | 72230365 | 72230440 | 72220053 |
| KLHL2 | chr4 | + | 165219933 | 165220059 | 165238777 | 165238899 | 165207617 |
| C11orf49 | chr11 | + | 46987207 | 46987310 | 47052387 | 47052518 | 46936746 |
| MKRN1 | chr7 | − | 140459706 | 140459936 | 140471882 | 140472011 | 140459006 |
| RP11-411B6.6 | chr10 | − | 100516873 | 100516960 | 100523976 | 100524139 | 100516095 |
| ZNF385A | chr12 | − | 54375843 | 54375954 | 54384427 | 54384564 | 54373972 |
| VARS | chr6 | − | 31781480 | 31781606 | 31781690 | 31781761 | 31781018 |
| TPM2 | chr9 | − | 35684731 | 35684807 | 35685063 | 35685139 | 35684487 |
| SLC27A3 | chr1 | + | 153778462 | 153778553 | 153778686 | 153778885 | 153778160 |
| SLC27A3 | chr1 | + | 153778462 | 153778553 | 153779113 | 153779211 | 153778160 |
| MICU3 | chr8 | + | 17090545 | 17090584 | 17098457 | 17098553 | 17086963 |

| | upstream | downstream | downstream | | | |
|---|---|---|---|---|---|---|
| geneSymbol | EE | ES | EE | PValue | FDR | type |
| NEGR1 | 71611146 | 71935078 | 71935311 | 1.739E−06 | 4.862E−04 | DN |
| RPL9 | 39454944 | 39458193 | 39458309 | 0.000E+00 | 0.000E+00 | DN |
| C17orf101 | 82405380 | 82418411 | 82418576 | 1.737E−03 | 4.254E−02 | UP |
| ARHGAP23 | 38497826 | 38500596 | 38500628 | 5.005E−04 | 1.948E−02 | DN |
| DUSP22 | 304661 | 345853 | 345928 | 1.550E−04 | 9.593E−03 | UP |
| ZCCHC17 | 31339048 | 31364031 | 31364953 | 2.769E−04 | 1.391E−02 | UP |
| ADHFE1 | 66432575 | 66444593 | 66444748 | 1.908E−03 | 4.485E−02 | UP |
| FHOD3 | 36693423 | 36730645 | 36730804 | 3.467E−05 | 3.549E−03 | UP |
| C1orf61 | 156407197 | 156429375 | 156429392 | 1.746E−06 | 4.862E−04 | DN |
| C1orf61 | 156407975 | 156429375 | 156429392 | 3.569E−05 | 3.549E−03 | DN |
| COMMD4 | 75336230 | 75338645 | 75338685 | 1.946E−03 | 4.543E−02 | UP |
| ANKS3 | 4714186 | 4734155 | 4734377 | 8.043E−04 | 2.532E−02 | UP |
| C6orf136 | 30647846 | 30652647 | 30652717 | 4.159E−05 | 3.736E−03 | UP |
| C6orf136 | 30647303 | 30652647 | 30652717 | 7.573E−04 | 2.446E−02 | UP |
| C6orf136 | 30651082 | 30652801 | 30652915 | 1.471E−03 | 3.777E−02 | UP |
| SYT5 | 55175339 | 55178962 | 55179390 | 2.087E−05 | 2.527E−03 | UP |
| ATP2B1 | 89634903 | 89655678 | 89656067 | 5.665E−08 | 3.155E−05 | UP |
| RPS24 | 78037304 | 78040203 | 78040225 | 3.686E−09 | 2.566E−06 | DN |
| RALYL | 84774654 | 84862295 | 84862453 | 1.518E−03 | 3.840E−02 | UP |
| SNAP25 | 10277726 | 10296924 | 10297050 | 0.000E+00 | 0.000E+00 | UP |
| SNAP25 | 10284772 | 10296924 | 10297050 | 0.000E+00 | 0.000E+00 | DN |
| BFAR | 14648592 | 14661891 | 14662065 | 9.053E−04 | 2.726E−02 | DN |
| UPF3B | 119841778 | 119851494 | 119851601 | 6.869E−05 | 5.212E−03 | UP |
| C12orf10 | 53300262 | 53306679 | 53306861 | 6.151E−04 | 2.196E−02 | UP |
| SCG5 | 32643818 | 32696513 | 32697098 | 4.010E−04 | 1.761E−02 | UP |
| N4BP2L2 | 32518003 | 32535768 | 32537027 | 6.521E−05 | 5.115E−03 | UP |
| SERPING1 | 57598321 | 57606407 | 57606547 | 8.771E−04 | 2.670E−02 | UP |
| WDR6 | 49007531 | 49014382 | 49014499 | 1.018E−04 | 6.994E−03 | UP |
| PDLIM2 | 22585162 | 22589595 | 22589741 | 1.874E−04 | 1.084E−02 | DN |
| PTK2 | 140659678 | 140674297 | 140674404 | 3.073E−05 | 3.316E−03 | DN |
| NPM2 | 22033223 | 22036492 | 22036526 | 3.863E−05 | 3.630E−03 | UP |
| ZNF75A | 3312768 | 3317189 | 3317925 | 8.204E−04 | 2.567E−02 | DN |
| RABEPK | 125200906 | 125227909 | 125228059 | 1.272E−05 | 1.915E−03 | UP |
| RABEPK | 125203066 | 125227909 | 125228059 | 1.459E−05 | 2.032E−03 | UP |
| DHRSX | 2266947 | 2425196 | 2425304 | 1.431E−03 | 3.741E−02 | UP |
| ANAPC7 | 110376216 | 110382842 | 110382960 | 4.634E−04 | 1.857E−02 | UP |
| PUS1 | 131932312 | 131943538 | 131943859 | 1.448E−04 | 9.062E−03 | UP |
| FAM153B | 176103297 | 176106559 | 176106590 | 6.957E−06 | 1.250E−03 | DN |
| CSTF3 | 33087141 | 33096305 | 33096408 | 2.764E−06 | 6.998E−04 | UP |
| PHF20 | 35801605 | 35863012 | 35863400 | 8.288E−04 | 2.579E−02 | DN |
| APLP1 | 35877825 | 35878889 | 35878952 | 1.405E−03 | 3.713E−02 | UP |
| SIRPA | 1915455 | 1927874 | 1927899 | 6.389E−04 | 2.252E−02 | DN |
| MAP4 | 47928350 | 48088772 | 48088839 | 1.510E−03 | 3.840E−02 | UP |
| EDEM2 | 35126375 | 35137879 | 35138005 | 3.036E−04 | 1.458E−02 | DN |
| PPP2R2B | 146698144 | 147081058 | 147081136 | 5.313E−04 | 2.013E−02 | DN |
| ATP5B | 56643587 | 56645836 | 56646068 | 1.466E−03 | 3.777E−02 | UP |
| BIN1 | 127050912 | 127059010 | 127059155 | 1.015E−03 | 2.991E−02 | UP |
| BIN1 | 127051243 | 127059010 | 127059155 | 1.150E−03 | 3.236E−02 | UP |
| NUP205 | 135628111 | 135637930 | 135638059 | 1.790E−03 | 4.320E−02 | UP |
| DNAJB6 | 157337144 | 157366501 | 157366561 | 4.983E−04 | 1.948E−02 | UP |
| SYT1 | 79047362 | 79292007 | 79292130 | 7.380E−04 | 2.434E−02 | UP |
| METTL9 | 21599898 | 21624930 | 21625115 | 1.828E−03 | 4.336E−02 | UP |
| HDAC10 | 50249964 | 50250770 | 50250905 | 5.494E−04 | 2.053E−02 | DN |
| SIPA1L2 | 232402473 | 232415493 | 232415625 | 3.760E−04 | 1.703E−02 | UP |
| EPB41L2 | 130863718 | 130885095 | 130885268 | 2.137E−05 | 2.532E−03 | DN |
| C20orf27 | 3755607 | 3767662 | 3768387 | 1.023E−03 | 3.000E−02 | UP |
| EI24 | 125578257 | 125582345 | 125582420 | 1.560E−05 | 2.120E−03 | UP |
| EI24 | 125578257 | 125582345 | 125582420 | 3.358E−06 | 8.132E−04 | DN |
| UBXN4 | 135770735 | 135778947 | 135779079 | 4.250E−04 | 1.797E−02 | DN |
| ANKRD11 | 89291183 | 89418283 | 89418368 | 8.347E−04 | 2.583E−02 | DN |
| LUC7L | 208188 | 229278 | 229450 | 8.088E−11 | 9.010E−08 | UP |
| SNRPN | 24962209 | 24976876 | 24977029 | 2.617E−04 | 1.363E−02 | UP |
| SNRPN | 24962209 | 24977777 | 24977916 | 6.910E−04 | 2.361E−02 | UP |
| KIAA1191 | 176348356 | 176361601 | 176361765 | 2.122E−03 | 4.772E−02 | DN |
| KIAA1191 | 176350737 | 176361601 | 176361807 | 1.150E−03 | 3.236E−02 | UP |
| TBC1D7 | 13305187 | 13316570 | 13316708 | 4.007E−04 | 1.761E−02 | UP |
| ABHD12 | 25295030 | 25306832 | 25306915 | 6.624E−04 | 2.295E−02 | UP |
| LMF2 | 50504984 | 50505402 | 50505537 | 6.000E−05 | 4.914E−03 | UP |
| CLTA | 36197588 | 36211602 | 36212056 | 9.196E−04 | 2.738E−02 | UP |
| RALY | 34076815 | 34079909 | 34080271 | 8.627E−04 | 2.640E−02 | DN |
| ATP6V1H | 53756656 | 53801798 | 53801896 | 7.853E−05 | 5.755E−03 | UP |
| COX6C | 99878265 | 99893638 | 99894062 | 5.993E−06 | 1.113E−03 | UP |
| MBD1 | 50272955 | 50274976 | 50275045 | 5.855E−04 | 2.160E−02 | UP |
| C6orf1 | 34247149 | 34248978 | 34249040 | 7.739E−05 | 5.748E−03 | UP |
| RTN1 | 59607492 | 59870389 | 59870420 | 5.785E−05 | 4.810E−03 | UP |
| AKIRIN1 | 38991600 | 39003346 | 39003418 | 2.719E−04 | 1.390E−02 | UP |
| MPDZ | 13113054 | 13119501 | 13119649 | 8.046E−04 | 2.532E−02 | UP |
| MKKS | 10408803 | 10434107 | 10434222 | 1.093E−03 | 3.137E−02 | DN |
| ASPDH | 50511773 | 50512926 | 50513011 | 6.048E−04 | 2.187E−02 | UP |
| ASPDH | 50511773 | 50514477 | 50514690 | 1.291E−03 | 3.525E−02 | UP |
| MON1A | 49909154 | 49911525 | 49912011 | 4.393E−04 | 1.812E−02 | UP |
| MBP | 76984894 | 76989955 | 76990060 | 2.950E−04 | 1.454E−02 | UP |
| SULT1A1 | 28607077 | 28608707 | 28608859 | 4.578E−05 | 4.048E−03 | DN |
| PKM | 72200655 | 72206727 | 72206880 | 3.565E−06 | 8.274E−04 | UP |
| MOG | 29657297 | 29667642 | 29667663 | 2.052E−04 | 1.115E−02 | UP |
| RAB6A | 73716362 | 73720845 | 73720899 | 1.278E−03 | 3.506E−02 | DN |
| TMX2 | 57737668 | 57738663 | 57738770 | 1.974E−03 | 4.563E−02 | UP |
| PLEKHA6 | 204222779 | 204228727 | 204228861 | 2.087E−03 | 4.744E−02 | UP |
| TMEM185A | 149600479 | 149611286 | 149611463 | 1.334E−03 | 3.606E−02 | DN |
| SNHG5 | 85666147 | 85678135 | 85678188 | 1.371E−03 | 3.671E−02 | DN |
| UPF3A | 114281846 | 114286518 | 114286629 | 3.643E−05 | 3.560E−03 | UP |
| UPF3A | 114291792 | 114304788 | 114305808 | 4.299E−04 | 1.797E−02 | DN |
| NDUFA8 | 122144378 | 122159626 | 122159819 | 1.831E−04 | 1.075E−02 | UP |
| EIF3L | 37876011 | 37888425 | 37888479 | 1.826E−03 | 4.336E−02 | UP |
| RIMS1 | 72259111 | 72291933 | 72292046 | 5.193E−04 | 1.995E−02 | UP |
| RIMS1 | 72259111 | 72291933 | 72292046 | 6.010E−04 | 2.187E−02 | UP |
| RIMS1 | 72260767 | 72291933 | 72292046 | 1.143E−04 | 7.671E−03 | UP |
| TSPAN3 | 77054279 | 77070891 | 77071109 | 3.548E−04 | 1.633E−02 | UP |
| WNK1 | 897681 | 907846 | 908034 | 4.324E−04 | 1.797E−02 | DN |
| LSM14B | 62126439 | 62130529 | 62130691 | 6.244E−05 | 5.040E−03 | DN |
| MCCC1 | 183086788 | 183115473 | 183116075 | 1.085E−03 | 3.136E−02 | DN |
| SPOCK3 | 166742059 | 166889129 | 166889244 | 1.762E−03 | 4.286E−02 | UP |
| IGSF8 | 160092695 | 160098408 | 160098517 | 6.846E−05 | 5.212E−03 | UP |
| SLC25A48 | 135871718 | 135888031 | 135889770 | 7.386E−04 | 2.434E−02 | UP |
| MDK | 46381183 | 46382586 | 46382748 | 9.214E−04 | 2.738E−02 | UP |
| HK1 | 69380095 | 69400990 | 69401443 | 2.125E−03 | 4.772E−02 | UP |
| TFPT | 54108395 | 54115246 | 54115288 | 2.094E−04 | 1.122E−02 | UP |
| GJB6 | 20229749 | 20231381 | 20231505 | 1.052E−06 | 3.256E−04 | UP |
| GJB6 | 20229749 | 20231381 | 20231505 | 8.783E−05 | 6.272E−03 | UP |
| MYT1L | 1912110 | 1979164 | 1979227 | 1.922E−05 | 2.433E−03 | DN |
| VIPR2 | 159031869 | 159034580 | 159034650 | 1.502E−03 | 3.838E−02 | DN |
| POLR2G | 62761660 | 62765181 | 62765232 | 4.439E−04 | 1.818E−02 | UP |
| VEZT | 95274889 | 95294271 | 95294372 | 8.832E−04 | 2.674E−02 | UP |
| CUX2 | 111296539 | 111306920 | 111307112 | 9.242E−04 | 2.738E−02 | DN |
| NR1H3 | 47261449 | 47267912 | 47268026 | 9.695E−05 | 6.813E−03 | UP |
| R3HDM2 | 57303217 | 57430719 | 57430808 | 3.477E−07 | 1.210E−04 | DN |
| ANAPC16 | 72220379 | 72233000 | 72235858 | 1.793E−03 | 4.320E−02 | DN |
| KLHL2 | 165207902 | 165263196 | 165263359 | 1.523E−03 | 3.840E−02 | UP |
| C11orf49 | 46936851 | 47137644 | 47137729 | 1.834E−04 | 1.075E−02 | UP |
| MKRN1 | 140459233 | 140479159 | 140479499 | 2.033E−04 | 1.115E−02 | UP |
| RP11-411B6.6 | 100516219 | 100526398 | 100526554 | 1.344E−03 | 3.617E−02 | DN |
| ZNF385A | 54374135 | 54391234 | 54391281 | 7.685E−04 | 2.446E−02 | UP |
| VARS | 31781123 | 31781846 | 31781952 | 5.424E−04 | 2.041E−02 | UP |
| TPM2 | 35684550 | 35685268 | 35685339 | 1.344E−04 | 8.602E−03 | DN |
| SLC27A3 | 153778355 | 153779113 | 153779211 | 1.218E−07 | 5.653E−05 | UP |
| SLC27A3 | 153778355 | 153779342 | 153779473 | 8.629E−06 | 1.373E−03 | UP |
| MICU3 | 17087035 | 17104390 | 17104491 | 1.704E−03 | 4.201E−02 | DN |

TABLE 27
FXS_VS_TD_A3SS

| | | | longExonStart_{—} | longExon | | |
|---|---|---|---|---|---|---|
| geneSymbol | chr | strand | 0base | End | shortES | shortEE |
| PMF1-BGLAP | chr1 | + | 156236287 | 156236483 | 156236348 | 156236483 |
| KLHDC4 | chr16 | − | 87707811 | 87708075 | 87707811 | 87708072 |
| PFN2 | chr3 | − | 149964903 | 149966586 | 149964903 | 149966264 |
| CAPN3 | chr15 | + | 42411746 | 42412317 | 42412085 | 42412317 |
| SYT1 | chr12 | + | 79292007 | 79292130 | 79292016 | 79292130 |
| ISOC2 | chr19 | − | 55455259 | 55455378 | 55455259 | 55455330 |
| MGRN1 | chr16 | + | 4686258 | 4690974 | 4688795 | 4690974 |
| IFI27 | chr14 | + | 94115780 | 94115942 | 94115789 | 94115942 |
| PAM | chr5 | + | 103028181 | 103028238 | 103028184 | 103028238 |
| CSNK1G3 | chr5 | + | 123590409 | 123590558 | 123590412 | 123590558 |
| TMEM132A | chr11 | + | 60931685 | 60931884 | 60931688 | 60931884 |
| STRN4 | chr19 | − | 46727451 | 46727567 | 46727451 | 46727546 |
| TNNT2 | chr1 | − | 201362385 | 201362394 | 201362385 | 201362391 |
| PSIP1 | chr9 | − | 15470635 | 15471311 | 15470635 | 15471231 |
| CLK2 | chr1 | − | 155268707 | 155268795 | 155268707 | 155268792 |
| ARL6IP4 | chr12 | + | 122981570 | 122981879 | 122981594 | 122981879 |
| ARL6IP4 | chr12 | + | 122981570 | 122981879 | 122981594 | 122981879 |
| NOLC1 | chr10 | + | 102157185 | 102157328 | 102157188 | 102157328 |

| geneSymbol | flankingES | flankingEE | PValue | FDR | type |
|---|---|---|---|---|---|
| PMF1-BGLAP | 156233627 | 156233728 | 1.544E−03 | 3.730E−02 | UP |
| KLHDC4 | 87708349 | 87708466 | 6.248E−04 | 2.071E−02 | DN |
| PFN2 | 149968357 | 149968550 | 5.841E−04 | 1.998E−02 | UP |
| CAPN3 | 42411286 | 42411345 | 2.978E−04 | 1.258E−02 | UP |
| SYT1 | 79285786 | 79285971 | 1.756E−08 | 9.459E−06 | DN |
| ISOC2 | 55456348 | 55456489 | 1.271E−04 | 6.845E−03 | DN |
| MGRN1 | 4683842 | 4683932 | 1.243E−04 | 6.845E−03 | UP |
| IFI27 | 94114850 | 94114880 | 7.949E−07 | 2.447E−04 | UP |
| PAM | 103019789 | 103019843 | 2.152E−03 | 4.592E−02 | DN |
| CSNK1G3 | 123588426 | 123588511 | 2.068E−03 | 4.468E−02 | DN |
| TMEM132A | 60930509 | 60930659 | 1.625E−03 | 3.764E−02 | DN |
| STRN4 | 46727893 | 46728007 | 1.891E−03 | 4.173E−02 | UP |
| TNNT2 | 201363295 | 201363406 | 6.491E−04 | 2.119E−02 | DN |
| PSIP1 | 15472631 | 15472750 | 5.779E−04 | 1.998E−02 | DN |
| CLK2 | 155269487 | 155269716 | 1.484E−03 | 3.634E−02 | UP |
| ARL6IP4 | 122981128 | 122981266 | 1.179E−07 | 5.083E−05 | DN |
| ARL6IP4 | 122981128 | 122981299 | 3.309E−11 | 7.131E−08 | DN |
| NOLC1 | 102157018 | 102157074 | 1.577E−03 | 3.730E−02 | UP |

TABLE 28
FXS_VS_TD_A5SS

| | | | longExon | longExon | | |
|---|---|---|---|---|---|---|
| geneSymbol | chr | strand | Start_0base | End | shortES | shortEE |
| APBA2 | chr15 | + | 28921639 | 28921749 | 28921639 | 28921718 |
| NDUFAF6 | chr8 | + | 95031994 | 95032098 | 95031994 | 95032094 |
| ADD1 | chr4 | + | 2904763 | 2905108 | 2904763 | 2905015 |
| FGFR1 | chr8 | − | 38428345 | 38428435 | 38428351 | 38428435 |
| GAS5 | chr1 | − | 173865470 | 173865547 | 173865509 | 173865547 |
| CES2 | chr16 | + | 66942647 | 66942785 | 66942647 | 66942737 |
| C6orf1 | chr6 | − | 34247680 | 34247864 | 34247740 | 34247864 |
| STRA13 | chr17 | − | 82019292 | 82019381 | 82019307 | 82019381 |
| HMGN3 | chr6 | − | 79202275 | 79202389 | 79202316 | 79202389 |
| RPP21 | chr6 | + | 30346431 | 30346564 | 30346431 | 30346557 |

| geneSymbol | flankingES | flankingEE | PValue | FDR | type |
|---|---|---|---|---|---|
| APBA2 | 28995752 | 28995806 | 2.703E−03 | 4.259E−02 | UP |
| NDUFAF6 | 95035453 | 95035576 | 1.090E−04 | 5.776E−03 | DN |
| ADD1 | 2907742 | 2907844 | 3.054E−04 | 1.061E−02 | UP |
| FGFR1 | 38427920 | 38428093 | 2.647E−03 | 4.259E−02 | UP |
| GAS5 | 173865228 | 173865282 | 3.806E−05 | 3.136E−03 | UP |
| CES2 | 66943298 | 66943371 | 2.099E−04 | 8.567E−03 | UP |
| C6orf1 | 34247466 | 34247492 | 1.785E−03 | 3.394E−02 | UP |
| STRA13 | 82018702 | 82019219 | 8.915E−04 | 2.138E−02 | UP |
| HMGN3 | 79201244 | 79201726 | 2.580E−03 | 4.247E−02 | DN |
| RPP21 | 30346712 | 30346857 | 1.323E−03 | 2.753E−02 | UP |

For additional information of Exemplification, see Shah et al., Antisense oligonucleotide rescue of CGG expansion-dependent FMR1 mis-splicing in fragile X syndrome restores FMRP, Proc Natl Acad Sci USA. 120(27):e2302534120 (2023), the contents of which, including Supporting Information, are incorporated herein by reference in their entirety.

### Embodiments

1. A method of treating a fragile X-associated disorder, comprising administering to a subject in need thereof, a therapeutically effective amount of an agent that modulates splicing of Fragile X Mental Retardation 1 (FMR1) gene, thereby treating the fragile X-associated disorder in the subject.
2. The method of Embodiment 1, wherein the fragile X-associated disorder is fragile X syndrome (FXS), fragile X-associated primary ovarian insufficiency (FXPOI), or fragile X-associated tremor/ataxia syndrome (FXTAS).
3. The method of Embodiment 1 or 2, wherein the agent increases splicing and/or expression of isoform 1 of the FMR1 gene, decreases splicing and/or expression of isoform 12 of the FMR1 gene, or a combination thereof.
4. The method of Embodiment 3, wherein the agent increases isoform 1 of the FMR1 gene by about 75%.
5. The method of Embodiment 3 or 4, wherein the agent decreases isoform 12 of the FMR1 gene by about 30%.
6. The method of any one of Embodiments 1-5, wherein the agent is a polynucleotide, optionally, wherein the polynucleotide is an antisense oligonucleotide (ASO).
7. The method of Embodiment 6, wherein the polynucleotide is a DNA polynucleotide or an RNA polynucleotide.
8. The method of Embodiment 6, wherein the polynucleotide is a small interfering RNA (siRNA), a short hairpin RNA (shRNA), an antisense DNA, an antisense RNA, a microRNA (miRNA), an antagomir, or a guide RNA (gRNA).
9. The method of any one of Embodiments 6-8, wherein the length of the polynucleotide is about 18-22 nucleotides.
10. The method of any one of Embodiments 6-9, wherein the polynucleotide comprises a nucleotide sequence that is complementary to a portion of the FMR1 gene transcript.
11. The method of Embodiment 10, wherein the polynucleotide comprises a nucleotide sequence that is at least 80% identical to at least a portion of the pseudo exon of the FMR1 gene (SEQ ID NO:19), at least 80% identical to at least a portion of the junction of intron 1 and the pseudo exon, or both.
12. The method of Embodiment 11, wherein the nucleotide sequence is at least 80% identical to:

(W-704)
(SEQ ID NO: 1)
AGAAGCCAAAGGAGACCTGA,
(W-705)
(SEQ ID NO: 2)
AAAGAGAAGCCAAAGGAGAC,
(W-706)
(SEQ ID NO: 3)
CTAGACCGGAAAAGAGAAGCCA,
(W-707)
(SEQ ID NO: 4)
ATGCTAGACCGGAAAAGAGAA,
(W-708)
(SEQ ID NO: 5)
CAATGCTAGACCGGAAAAGA,
(W-709)
(SEQ ID NO: 6)
AAGTCCCAATGCTAGACCGGA,
(W-710)
(SEQ ID NO: 7)
TCTCCGAAGTCCCAATGCTA,
(W-711)
(SEQ ID NO: 8)
GAGCTCTCCGAAGTCCCA,
(W-712)
(SEQ ID NO: 9)
AGAACAGTGGAGCTCTCCGA,
(W-713)
(SEQ ID NO: 10)
CGCCCAGAACAGTGGAGCTC,
or
(W-714)
(SEQ ID NO: 11)
CCTCGCCCAGAACAGTGGAG.

13. The method of Embodiment 12, wherein the nucleotide sequence is identical to:

(W-704)
(SEQ ID NO: 1)
AGAAGCCAAAGGAGACCTGA,
(W-705)
(SEQ ID NO: 2)
AAAGAGAAGCCAAAGGAGAC,
(W-706)
(SEQ ID NO: 3)
CTAGACCGGAAAAGAGAAGCCA,
(W-707)
(SEQ ID NO: 4)
ATGCTAGACCGGAAAAGAGAA,
(W-708)
(SEQ ID NO: 5)
CAATGCTAGACCGGAAAAGA,
(W-709)
(SEQ ID NO: 6)
AAGTCCCAATGCTAGACCGGA,
(W-710)
(SEQ ID NO: 7)
TCTCCGAAGTCCCAATGCTA,
(W-711)
(SEQ ID NO: 8)
GAGCTCTCCGAAGTCCCA),
(W-712)
(SEQ ID NO: 9)
AGAACAGTGGAGCTCTCCGA,
(W-713)
(SEQ ID NO: 10)
CGCCCAGAACAGTGGAGCTC,
or
(W-714)
(SEQ ID NO: 11)
CCTCGCCCAGAACAGTGGAG.

14. The method of Embodiment 13, comprising administering to the subject a polynucleotide comprising the nucleotide sequence of CGCCCAGAACAGTGGAGCTC (SEQ ID NO:10) (W-713), a polynucleotide comprising the nucleotide sequence of CCTCGCCCAGAACAGTGGAG (SEQ ID NO:11) (W-714), or both.
15. The method of any one of Embodiments 6-14, wherein the polynucleotide is modified, optionally, wherein the polynucleotide is modified with one or more locked nucleic acid (LNA) nucleotides, one or more 2′-modified ribonucleotides, one or more morpholino nucleotides, or a combination thereof.
16. The method of Embodiment 15, wherein the modification is a modification of a ribose group, a phosphate group, a nucleobase, or a combination thereof.
17. The method of Embodiment 15, wherein the polynucleotide is chemically modified to increase the nuclease resistance, to prevent RNase H cleavage of the complementary RNA strand, to increase cellular uptake, or a combination thereof.
18. The method of Embodiment 15, wherein the polynucleotide is chemically modified to comprise a locked nucleic acid (LNA), an ethyl-constrained nucleotide, a 2′-(S)-constrained ethyl (S-cEt) nucleotide, a constrained MOE, a 2′-0,4′-C-aminomethylene bridged nucleic acid (2′,4′-BNANC), an alpha-L-locked nucleic acid, and a tricyclo-DNA, or a combination thereof.
19. The method of Embodiment 16, wherein the chemical modification is a modification of a ribose group and wherein the modification of the ribose group comprises 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH₂, a constrained nucleotide, a tricyclo-DNA modification, or a combination thereof.
20. The method of Embodiment 16, wherein the chemical modification is a modification of a phosphate group and wherein the modification of the phosphate group comprises a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof.
21. The method of Embodiment 16, wherein the chemical modification is a modification of a nucleobase and wherein the modification of the nucleobase comprises 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, halogenated aromatic groups, or a combination thereof.
22. The method of Embodiment 15, wherein the chemical modification is a modification of the polynucleotide sugar-phosphate backbone.
23. The method of Embodiment 22, wherein the sugar-phosphate backbone is replaced with a phosphorodiamidate mopholino (PMO), a peptide nucleic acid or other pseudopeptide backbone.
24. The method of Embodiment 15, wherein the polynucleotide is a phosphorothioate-modified polynucleotide, such as a polynucleotide where each internucleotide linkage is a phosphorothioate, or wherein at least half of the internucleotide linkages are phosphorothioate.
25. The method of any one of Embodiments 1-24, wherein the subject is a human who has, or is predisposed to have, FXS.
26. The method of Embodiment 25, wherein the subject comprises a CGG repeat expansion exceeding 200 repeats in the 5′ untranslated region of the FMR1 gene.
27. The method of any one of Embodiments 1-24, wherein the subject is a human who has, or is predisposed to have, FXTAS.
28. The method of Embodiment 27, wherein the subject comprises a CGG repeat expansion of about 50 to about 200 repeats in the 5′ untranslated region of the FMR1 gene.
29. The method of any one of Embodiments 25-28, wherein the CGG repeat expansion is partially methylated.
30. The method of any one of Embodiments 25-28, wherein the CGG repeat expansion is fully methylated.
31. The method of any one of Embodiments 25-30, wherein the subject has an increased level of isoform 12 of the FMR1 gene.
32. The method of any one of Embodiments 25-31, wherein the human is a male.
33. The method of any one of Embodiments 25-32, wherein the subject is about 2-11, 4-17, 12-18, or 18-50 years of age.
34. The method of any one of Embodiments 6-33, wherein the polynucleotide is administered intravenously, intra-arterially, intrathecally, intraventricularly, intramuscularly, intradermally, subcutaneously, intracranially, or spinally.
35. The method of any one of Embodiments 1-34, further comprising administering to the subject a therapeutically effective amount of a DNA-demethylating compound or DNA demethylase prior to administering the polynucleotide.
36. The method of Embodiment 35, wherein the DNA-demethylating compound or DNA demethylase is administered in an amount sufficient to demethylate about 25-50% of FMR1 gene.
37. The method of any one of Embodiments 1-36, wherein treating FXS includes slowing progression of FXS, alleviating one or more signs or symptoms of FXS, preventing one or more signs or symptoms of FXS, or a combination thereof.
38. A method of modulating Fragile X Mental Retardation 1 (FMR1) splicing and/or expression in a cell, comprising contacting the cell with a polynucleotide under conditions whereby the polynucleotide is introduced into the cell, wherein the polynucleotide increases splicing and/or expression of isoform 1 of the FMR1 gene, decreases splicing and/or expression of isoform 12 of the FMR1 gene, or a combination thereof.
39. The method of Embodiment 38, wherein the cell is an in vitro cell or an ex vivo cell.
40. The method of Embodiment 39, wherein the cell is an induced pluripotent stem cell (iPSC)-derived neuron from a human who has or is predisposed to have FXS, a primary human cell, or a cell line.
41. The method of Embodiment 40, wherein the cell is a cell of a subject.
42. The method of Embodiment 41, wherein the cell is allogeneic.
43. The method of Embodiment 41, wherein the cell is autologous or syngeneic.
44. A polynucleotide, comprising a nucleotide sequence that is complementary to a portion of the FMR1 gene transcript.
45. The polynucleotide of Embodiment 44, wherein the nucleotide sequence is at least 80% identical to at least a portion of iso12 of the FMR1 gene, at least 80% identical to at least a portion of the junction of intron 1 and iso12 of the FMR1 gene, or both.
46. The polynucleotide of Embodiment 45, wherein the nucleotide sequence is at least 80% identical to:

(W-704)
(SEQ ID NO: 1)
AGAAGCCAAAGGAGACCTGA,
(W-705)
(SEQ ID NO: 2)
AAAGAGAAGCCAAAGGAGAC,
(W-706)
(SEQ ID NO: 3)
CTAGACCGGAAAAGAGAAGCCA,
(W-707)
(SEQ ID NO: 4)
ATGCTAGACCGGAAAAGAGAA,
(W-708)
(SEQ ID NO: 5)
CAATGCTAGACCGGAAAAGA,
(W-709)
(SEQ ID NO: 6)
AAGTCCCAATGCTAGACCGGA,
(W-710)
(SEQ ID NO: 7)
TCTCCGAAGTCCCAATGCTA,
(W-711)
(SEQ ID NO: 8)
GAGCTCTCCGAAGTCCCA,
(W-712)
(SEQ ID NO: 9)
AGAACAGTGGAGCTCTCCGA,
(W-713)
(SEQ ID NO: 10)
CGCCCAGAACAGTGGAGCTC,
or
(W-714)
(SEQ ID NO: 11)
CCTCGCCCAGAACAGTGGAG.

47. A pharmaceutical composition, comprising the polynucleotide of any one of Embodiments 44-46, and one or more pharmaceutically acceptable excipients, diluents, or carriers.
48. A microarray for the detection of a fragile X-associated disorder, comprising at least one nucleic acid probe immobilized on a solid substrate, said probe comprising a nucleic acid sequence complementary to a portion of the FMR1 gene transcript.
49. The microarray of Embodiment 48, wherein the nucleotide sequence has at least 80% sequence identity to at least a portion of Exon 2 of FMR1-217, at least a portion of the junction of intron 1-2 and Exon 2 of FMR1-217, or both.
50. The microarray of Embodiment 49, wherein the nucleotide sequence is at least 80% identical to:

(W-704)
(SEQ ID NO: 1)
AGAAGCCAAAGGAGACCTGA,
(W-705)
(SEQ ID NO: 2)
AAAGAGAAGCCAAAGGAGAC,
(W-706)
(SEQ ID NO: 3)
CTAGACCGGAAAAGAGAAGCCA,
(W-707)
(SEQ ID NO: 4)
ATGCTAGACCGGAAAAGAGAA,
(W-708)
(SEQ ID NO: 5)
CAATGCTAGACCGGAAAAGA,
(W-709)
(SEQ ID NO: 6)
AAGTCCCAATGCTAGACCGGA,
(W-710)
(SEQ ID NO: 7)
TCTCCGAAGTCCCAATGCTA,
(W-711)
(SEQ ID NO: 8)
GAGCTCTCCGAAGTCCCA,
(W-712)
(SEQ ID NO: 9)
AGAACAGTGGAGCTCTCCGA,
(W-713)
(SEQ ID NO: 10)
CGCCCAGAACAGTGGAGCTC,
or
(W-714)
(SEQ ID NO: 11)
CCTCGCCCAGAACAGTGGAG.

### REFERENCES

1. J. C. Darnell, S. J. Van Driesche, C. Zhang, K. Y. S. Hung, A. Mele, C. E. Fraser, E. F. Stone, C. Chen, J. J. Fak, S. W. Chi, D. D. Licatalosi, J. D. Richter, R. B. Darnell, FMRP stalls ribosomal translocation on mRNAs linked to synaptic function and autism. Cell (2011), doi:10.1016/j.cell.2011.06.013.
2. T. Maurin, K. Lebrigand, S. Castagnola, A. Paquet, M. Jarjat, A. Popa, M. Grossi, F. Rage, B. Bardoni, HITS-CLIP in various brain areas reveals new targets and new modalities of RNA binding by fragile X mental retardation protein. Nucleic Acids Res. 46, 6344-6355 (2018).
3. M. Li, J. Shin, R. D. Risgaard, M. J. Parries, J. Wang, D. Chasman, S. Liu, S. Roy, A. Bhattacharyya, X. Zhao, Identification of FMR1-regulated molecular networks in human neurodevelopment. (2020), doi:10.1101/gr.251405.119.
4. G. Dölen, E. Osterweil, B. S. S. Rao, G. B. Smith, B. D. Auerbach, S. Chattarji, M. F. Bear, Correction of Fragile X Syndrome in Mice. Neuron 56, 955-962 (2007).
5. B. Liu, Y. Li, E. E. Stackpole, A. Novak, Y. Gao, Y. Zhao, X. Zhao, J. D. Richter, Regulatory discrimination of mRNAs by FMRP controls mouse adult neural stem cell differentiation. Proc. Natl. Acad. Sci. U.S.A (2018), doi:10.1073/pnas.1809588115.
6. H. Shu, E. Donnard, B. Liu, S. Jung, R. Wang, J. D. Richter, R. W. Ana-Lyzed, FMRP links optimal codons to mRNA stability in neurons. 117, 30400-30411 (2020).
7. E. J. Greenblatt, A. C. Spradling, Fragile X mental retardation 1 gene enhances the translation of large autism-related proteins.
8. J. D. Richter, X. Zhao, The molecular biology of FMRP: new insights into fragile X syndrome Nat. Rev. Neurosci. (2021), doi:10.1038/s41583-021-00432-0.
9. J. C. Darnell, S. J. Van Driesche, C. Zhang, K. Y. S. Hung, A. Mele, C. E. Fraser, E. F. Stone, C. Chen, J. J. Fak, S. W. Chi, D. D. Licatalosi, J. D. Richter, R. B. Darnell, FMRP stalls ribosomal translocation on mRNAs linked to synaptic function and autism. Cell (2011), doi:10.1016/j.cell.2011.06.013.
10. T. Udagawa, N. G. Farny, M. Jakovcevski, H. Kaphzan, J. M. Alarcon, S. Anilkumar, M. Ivshina, J. A. Hurt, K. Nagaoka, V. C. Nalavadi, L. J. Lorenz, G. J. Bassell, S. Akbarian, S. Chattarji, E. Klann, J. D. Richter, Genetic and acute CPEB1 depletion ameliorate fragile X pathophysiology. Nat. Med. 19, 1473 (2013).
11. S. Shah, G. Molinaro, B. Liu, R. Wang, K. M. Huber, J. D. Richter, FMRP Control of Ribosome Translocation Promotes Chromatin Modifications and Alternative Splicing of Neuronal Genes Linked to Autism. Cell Rep. (2020), doi:10.1016/j.celrep.2020.02.076.
12. S. Kim, H. Kim, N. Fong, B. Erickson, D. L. Bentley, Pre-mRNA splicing is a determinant of histone H3K36 methylation. Proc Natl Acad Sci USA 108, 13564-13569 (2011).
13. S. Shah, J. D. Richter, Do Fragile X Syndrome and Other Intellectual Disorders Converge at Aberrant Pre-mRNA Splicing? Front. Psychiatry 12, 715346 (2021).
14. S. Shen, J. W. Park, Z. Lu, L. Lin, M. D. Henry, Y. N. Wu, Q. Zhou, Y. Xing, rMATS: Robust and flexible detection of differential alternative splicing from replicate RNA-Seq data. Proc. Natl. Acad. Sci. (2014), doi:10.1073/pnas.1419161111.
15. E. Korb, M. Herre, I. Zucker-Scharff, J. Gresack, C. D. Allis, R. B. Darnell, Excess Translation of Epigenetic Regulators Contributes to Fragile X Syndrome and Is Alleviated by Brd4 Inhibition. Cell 170, 1209-1223.e20 (2017).
16. S. S. Tran, H. I. Jun, J. H. Bahn, A. Azghadi, G. Ramaswami, E. L. Van Nostrand, T. B. Nguyen, Y. H. E. Hsiao, C. Lee, G. A. Pratt, V. Martinez-Cerdeño, R. J. Hagerman, G. W. Yeo, D. H. Geschwind, X. Xiao, Widespread RNA editing dysregulation in brains from autistic individuals. Nat. Neurosci. (2019), doi:10.1038/s41593-018-0287-x.
17. D. Vershkov, N. Fainstein, S. Suissa, T. Golan-Lev, T. Ben-Hur, N. Benvenisty, FMR1 Reactivating Treatments in Fragile X iPSC-Derived Neural Progenitors In Vitro and In Vivo. Cell Rep. 26, 2531-2539.e4 (2019).
18. X. S. Liu, H. Wu, M. Krzisch, X. Wu, J. Graef, J. Muffat, D. Hnisz, C. H. Li, B. Yuan, C. Xu, Y. Li, D. Vershkov, A. Cacace, R. A. Young, R. Jaenisch, Rescue of Fragile X Syndrome Neurons by DNA Methylation Editing of the FMR1 Gene. Cell 172, 979-991.e6 (2018).
19. R. Esanov, N. S. Andrade, S. Bennison, C. Wahlestedt, Z. Zeier, The FMR1 promoter is selectively hydroxymethylated in primary neurons of fragile X syndrome patients. (2016), doi:10.1093/hmg/ddw311.
20. N. Raj, Z. T. McEachin, W. Harousseau, Y. Zhou, F. Zhang, M. E. Merritt-Garza, J. M. Taliaferro, M. Kalinowska, S. G. Marro, C. M. Hales, E. Berry-Kravis, M. W. Wolf-Ochoa, V. Martinez-Cerdeno, M. Wernig, L. Chen, E. Klann, S. T. Warren, P. Jin, Z. Wen, G. J. Bassell, Cell-type-specific profiling of human cellular models of fragile X syndrome reveal PI3K-dependent defects in translation and neurogenesis. Cell Rep. 35 (2021), doi:10.1016/J.CELREP.2021.108991.
21. E. Tabolacci, F. Palumbo, V. Nobile, G. Neri, Transcriptional reactivation of the FMR1 Gene. A possible approach to the treatment of the fragile X syndrome. Genes (Basel). 7, 1-16 (2016).
22. E. Tabolacci, G. Mancano, S. Lanni, F. Palumbo, M. Goracci, F. Ferrè, M. Helmer-Citterich, G. Neri, Genome-wide methylation analysis demonstrates that 5-aza-2-deoxycytidine treatment does not cause random DNA demethylation in fragile X syndrome cells. Epigenetics and Chromatin 9, 1-16 (2016).
23. M. P. Moazami, J. M. Rembetsy-Brown, F. Wang, P. M. Krishnamurthy, A. Weiss, M. Marosfoi, R. M. King, M. Motwani, H. Gray-Edwards, K. A. Fitzgerald, R. H. Brown, J. K. Watts, Quantifying and Mitigating Motor Phenotypes Induced by Antisense Oligonucleotides in the Central 1 Nervous System 2. (2021), doi:10.1101/2021.02.14.431096.
24. S. D. Sheridan, K. M. Theriault, S. A. Reis, F. Zhou, J. M. Madison, L. Daheron, J. F. Loring, S. J. Haggarty, Epigenetic characterization of the FMR1 gene and aberrant neurodevelopment in human induced pluripotent stem cell models of fragile X syndrome. PLoS One 6 (2011), doi:10.1371/journal.pone.0026203.
25. M. J. Gandal, P. Zhang, E. Hadjimichael, R. L. Walker, C. Chen, S. Liu, H. Won, H. Van Bakel, M. Varghese, Y. Wang, A. W. Shieh, J. Haney, S. Parhami, J. Belmont, M. Kim, P. M. Losada, Z. Khan, J. Mleczko, Y. Xia, R. Dai, D. Wang, Y. T. Yang, M. Xu, K. Fish, P. R. Hof, J. Warrell, D. Fitzgerald, K. White, A. E. Jaffe, M. A. Peters, M. Gerstein, C. Liu, L. M. Iakoucheva, D. Pinto, D. H. Geschwind, Transcriptome-wide isoform-level dysregulation in ASD, schizophrenia, and bipolar disorder. Science (80-). 362 (2018), doi:10.1126/science.aat8127.
26. M. Irimia, R. J. Weatheritt, J. D. Ellis, N. N. Parikshak, T. Gonatopoulos-Pournatzis, M. Babor, M. Quesnel-Vallières, J. Tapial, B. Raj, D. O'Hanlon, M. Barrios-Rodiles, M. J. E. Sternberg, S. P. Cordes, F. P. Roth, J. L. Wrana, D. H. Geschwind, B. J. Blencowe, A highly conserved program of neuronal microexons is misregulated in autistic brains. Cell (2014), doi:10.1016/j.cell.2014.11.035.
27. M. Quesnel-Vallières, Z. Dargaei, M. Irimia, T. Gonatopoulos-Pournatzis, J. Y. Ip, M. Wu, T. Sterne-Weiler, S. Nakagawa, M. A. Woodin, B. J. Blencowe, S. P. Cordes, Misregulation of an Activity-Dependent Splicing Network as a Common Mechanism Underlying Autism Spectrum Disorders. Mol. Cell (2016), doi:10.1016/j.molcel.2016.11.033.
28. M. Zafarullah, F. Tassone, Molecular Biomarkers in Fragile X Syndrome. (2019), doi:10.3390/brainsci9050096.
29. C. J. Westmark, The quest for fragile X biomarkers (2014; http://www.molcellped.com/content/1/1/1).
30. E. Berry-Kravis, D. Hessl, L. Abbeduto, A. L. Reiss, A. Beckel-Mitchener, T. K. Urv, O. Measures, W. Groups, M. Aman, K. Clapp, S. Hall, B. Kronk, A. Lachiewicz, R. Noll, L. Scahill, H. C. Hazlett, S. Hooper, B. King, A. Lightbody, R. Paylor, D. Posey, P. Wang, B. Zorovic, C. Erickson, R. Hagerman, W. Kaufmann, E. Klann, J. Piven, A. Reiss, L. Sikich, N. Tartaglia, M. Tranfaglia, Outcome Measures for Clinical Trials in Fragile X Syndrome. J Dev Behav Pediatr 34, 508-522 (2013).
31. M. Jasoliya, H. Bowling, I. C. Petrasic, B. Durbin-Johnson, E. Klann, A. Bhattacharya, R. Hagerman, F. Tassone, Blood-Based Biomarkers Predictive of Metformin Target Engagement in Fragile X Syndrome. (2020), doi:10.3390/brainsci10060361.
32. O. Dionne, F. Corbin, A new strategy to uncover fragile X proteomic biomarkers using the nascent proteome of peripheral blood mononuclear cells (PBMCs). Sci. Reports|11, 15148 (2021).
33. M. Zafarullah, H.-T. Tang, B. Durbin-Johnson, E. Fourie, D. Hessl, S. M. Rivera, FMR1 locus isoforms: potential biomarker candidates in fragile X-associated tremor/ataxia syndrome (fXtAS). 10, 11099 (2020).
34. P. Chiurazzi, M. G. Pomponi, R. Willemsen, B. A. Oostra, G. Neri, In vitro reactivation of the FMR1 gene involved in fragile X syndrome (1998; https://academic.oup.com/hmg/article/7/1/109/637850).
35. M. Derbis, E. Kul, D. Niewiadomska, M. Sekrecki, A. Piasecka, K. Taylor, R. K. Hukema, O. Stork, K. Sobczak, Short antisense oligonucleotides alleviate the pleiotropic toxicity of RNA harboring expanded CGG repeats. Nat. Commun. 12, 1-17 (2021).
36. C. M. Rodriguez, S. E. Wright, M. G. Kearse, J. M. Haenfler, B. N. Flores, Y. Liu, M. F. Ifrim, M. R. Glineburg, A. Krans, P. Jafar-nejad, M. A. Sutton, G. J. Bassell, J. M. Parent, F. Rigo, S. J. Barmada, P. K. Todd, repeats in regulating fragile X protein synthesis. Nat. Neurosci. 23, 386-397 (2020).
37. X. S. Liu, H. Wu, M. Krzisch, X. Wu, J. Graef, J. Muffat, D. Hnisz, C. H. Li, B. Yuan, C. Xu, Y. Li, D. Vershkov, A. Cacace, R. A. Young, R. Jaenisch, Rescue of Fragile X Syndrome Neurons by DNA Methylation Editing of the FMR1 Gene. Cell 172, 979-992.e6 (2018).
38. J. M. Haenfler, G. Skariah, C. M. Rodriguez, A. Monteiro da Rocha, J. M. Parent, G. D. Smith, P. K. Todd, R. Dahlhaus, K.-M. Yao, A. Plagge, P. Ramos-Ibeas, Targeted Reactivation of FMR1 Transcription in Fragile X Syndrome Embryonic Stem Cells. Physiol. Urol. (2018), doi:10.3389/fnmol.2018.00282.
39. N. Xie, H. Gong, J. A. Suhl, P. Chopra, T. Wang, S. T. Warren, Reactivation of FMR1 by CRISPR/Cas9-mediated deletion of the expanded CGG-repeat of the fragile X chromosome. PLoS One 11, 1-12 (2016).
40. P. C.-Y., H. T., L. D. R., S. J. J., L. J. S., Y. O., B. N., Reversion of FMR1 Methylation and Silencing by Editing the Triplet Repeats in Fragile X iPSC-Derived Neurons. Cell Rep. 13, 234-241 (2015).
41. R. S. Finkel, C. A. Chiriboga, J. Vajsar, J. W. Day, J. Montes, D. C. De Vivo, M. Yamashita, F. Rigo, G. Hung, E. Schneider, D. A. Norris, S. Xia, C. F. Bennett, K. M. Bishop, Treatment of infantile-onset spinal muscular atrophy with nusinersen: a phase 2, open-label, dose-escalation study. Lancet 388, 3017-3026 (2016).
42. D. G. W. Mulders Susan, Walt, Triplet-repeat oligonucleotide-mediated . . . n myotonic dystrophy Enhanced Reader.pdf (2009).
43. S. K. Pandey, T. M. Wheeler, S. L. Justice, A. Kim, H. S. Younis, D. Gattis, D. Jauvin, J. Puymirat, E. E. Swayze, S. M. Freier, C. F. Bennett, C. A. Thornton, A. R. Macleod, Identification and Characterization of Modified Antisense Oligonucleotides Targeting DMPK in Mice and Nonhuman Primates for the Treatment of Myotonic Dystrophy Type 1 s. J. Pharmacol. Exp. Ther. J Pharmacol Exp Ther 355, 329-340 (2015).
44. T. M. Wheeler, A. J. Leger, S. K. Pandey, A. R. Macleod, M. Nakamori, S. H. Cheng, B. M. Wentworth, C. F. Bennett, C. A. Thornton, Targeting nuclear RNA for in vivo correction of myotonic dystrophy. (2012), doi:10.1038/nature11362.
45. R. A. Smith, T. M. Miller, K. Yamanaka, B. P. Monia, T. P. Condon, G. Hung, C. S. Lobsiger, C. M. Ward, M. Mcalonis-Downes, H. Wei, E. V Wancewicz, C. F. Bennett, D. W. Cleveland, Antisense oligonucleotide therapy for neurodegenerative disease. J. Clin. Invest. 116 (2006), doi:10.1172/JC125424.
46. C. J. Donnelly, P. W. Zhang, J. T. Pham, A. R. Heusler, N. A. Mistry, S. Vidensky, E. L. Daley, E. M. Poth, B. Hoover, D. M. Fines, N. Maragakis, P. J. Tienari, L. Petrucelli, B. J. Traynor, J. Wang, F. Rigo, C. F. Bennett, S. Blackshaw, R. Sattler, J. D. Rothstein, RNA Toxicity from the ALS/FTD C90RF72 Expansion Is Mitigated by Antisense Intervention. Neuron 80, 415-428 (2013).
47. H. Tran, M. P. Moazami, H. Yang, D. McKenna-Yasek, C. L. Douthwright, C. Pinto, J. Metterville, M. Shin, N. Sanil, C. Dooley, A. Puri, A. Weiss, N. Wightman, H. Gray-Edwards, M. Marosfoi, R. M. King, T. Kenderdine, D. Fabris, R. Bowser, J. K. Watts, R. H. Brown Jr, Suppression of mutant C9orf72 expression by a potent mixed backbone antisense oligonucleotide. Nat Med. 2022 January; 28(1):117-124. doi:10.1038/s41591-021-01557-6.
48. L. A. Becker, B. Huang, G. Bieri, R. Ma, D. A. Knowles, P. Jafar-Nejad, J. Messing, H. J. Kim, A. Soriano, G. Auburger, S. M. Pulst, J. P. Taylor, F. Rigo, A. D. Gitler, Therapeutic reduction of ataxin-2 extends lifespan and reduces pathology in TDP-43 mice. Nature. (2017), doi:10.1038/nature22038.
49. Y. Q. Jiang, A. Andrade, D. Lipscombe, Spinal morphine but not ziconotide or gabapentin analgesia is affected by alternative splicing of voltage-gated calcium channel CaV2.2 pre-mRNA. Mol. Pain 9 (2013), doi:10.1186/1744-8069-9-67.
50. C. Milazzo, E. J. Mientjes, I. Wallaard, S. V. Rasmussen, K. D. Erichsen, T. Kakunuri, A. S. E. van der Sman, T. Kremer, M. T. Miller, M. C. Hoener, Y. Elgersma, Antisense oligonucleotide treatment rescues UBE3A expression and multiple phenotypes of an Angelman syndrome mouse model. JCI Insight 6 (2021), doi:10.1172/jci.insight.145991.
51. A. V Scott Dindot, S. Christian, W. J. Murphy, A. Berent, J. Panagoulias, A. Schlafer, J. Ballard, K. Radeva, R. Robinson, L. Myers, T. Jepp, H. Shaheen, P. Hillman, K. Konganti, A. Hillhouse, K. R. Bredemeyer, L. Black, J. Douville, the FIRE consortium, Development of an ASO therapy for Angelman syndrome by targeting an evolutionarily conserved region at the start of the UBE3A-AS transcript. bioRxiv (Posted Jul. 27, 2021). doi:10.1101/2021.07.27.453820.
52. G. H. Roid, M. Pomplun, in Contemporary intellectual assessment: Theories, tests, and issues, 3rd ed., (The Guilford Press, New York, NY, US, 2012), pp. 249-268.
53. S. M. Sansone, A. Schneider, E. Bickel, E. Berry-Kravis, C. Prescott, D. Hessl, Improving IQ measurement in intellectual disabilities using true deviation from population norms (2014; http://www.jneurodevdisorders.com/content/6/1/16).
54. Sparrow S S; Balla D A; Cicchetti D V; Harrison P L; Doll E A., Vineland adaptive behavior scales. Am. Guid. Serv. Circ. Pines; MN (1984).
55. O. Yukselen, O. Turkyilmaz, A. Ozturk, M. Garber, A. Kucukural, DolphinNext: A distributed data processing platform for high throughput genomics. bioRxiv (2019), doi:10.1101/689539.
56. F. Cunningham, J. E. Allen, J. Allen, J. Alvarez-Jarreta, M. R. Amode, I. M. Armean, O. Austine-Orimoloye, A. G. Azov, I. Barnes, R. Bennett, A. Berry, J. Bhai, A. Bignell, K. Billis, S. Boddu, L. Brooks, M. Charkhchi, C. Cummins, L. Da Rin Fioretto, C. Davidson, K. Dodiya, S. Donaldson, B. El Houdaigui, T. El Naboulsi, R. Fatima, C. G. Giron, T. Genez, J. G. Martinez, C. Guijarro-Clarke, A. Gymer, M. Hardy, Z. Hollis, T. Hourlier, T. Hunt, T. Juettemann, V. Kaikala, M. Kay, I. Lavidas, T. Le, D. Lemos, J. C. Marugán, S. Mohanan, A. Mushtaq, M. Naven, D. N. Ogeh, A. Parker, A. Parton, M. Perry, I. Pilizota, I. Prosovetskaia, M. P. Sakthivel, A. I. A. Salam, B. M. Schmitt, H. Schuilenburg, D. Sheppard, J. G. Perez-Silva, W. Stark, E. Steed, K. Sutinen, R. Sukumaran, D. Sumathipala, M. M. Suner, M. Szpak, A. Thormann, F. F. Tricomi, D. Urbina-Gómez, A. Veidenberg, T. A. Walsh, B. Walts, N. Willhoft, A. Winterbottom, E. Wass, M. Chakiachvili, B. Flint, A. Frankish, S. Giorgetti, L. Haggerty, S. E. Hunt, G. R. Iisley, J. E. Loveland, F. J. Martin, B. Moore, J. M. Mudge, M. Muffato, E. Perry, M. Ruffier, J. Tate, D. Thybert, S. J. Trevanion, S. Dyer, P. W. Harrison, K. L. Howe, A. D. Yates, D. R. Zerbino, P. Flicek, Ensembl 2022. Nucleic Acids Res. 50, D988-D995 (2022).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. An antisense oligonucleotide (ASO), wherein the ASO specifically binds a contiguous nucleotide sequence set forth in any one of SEQ ID NOs:24-42, and wherein the contiguous nucleotide sequence is at least 12 nucleotides in length.

2. (canceled)

3. The ASO of claim 1, wherein the ASO is no more than 100 nucleotides in length.

4. The ASO of claim 1, wherein the ASO is about 18-24 nucleotides in length.

5. The ASO of claim 1, wherein the ASO comprises:
at least one modification of a ribose or deoxyribose group,
at least one modification of a phosphate group,
at least one modification of a nucleobase,
at least one phosphodiester internucleotide linkage,
at least one phosphorothioate internucleotide linkage,
or any combination of the foregoing.

6. The ASO of claim 1, wherein the ASO is modified to comprise:
a locked nucleic acid (LNA), an ethyl-constrained nucleotide, a 2′-(S)-constrained ethyl (S-cEt) nucleotide, a constrained 2′-O-methoxyethyl (MOE), a 2′-O,4′-C-aminomethylene bridged nucleic acid (2′,4′-BNA(NC)), an alpha-L-locked nucleic acid, a tricyclo-DNA, or a combination thereof,
a ribose or deoxyribose group comprising a 2′-O-methyl, 2′-fluoro, 2′-deoxy, 2′-O-methoxyethyl (MOE), 2′-O-alkyl, 2′-O-alkoxy, 2′-O-alkylamino, or 2′-NH2 modification, a constrained nucleotide, a tricyclo-DNA modification, or a combination thereof,
a phosphate group comprising a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof,
a nucleobase comprising 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, halogenated aromatic groups, or a combination thereof,
a polynucleotide backbone comprising a sugar phosphate backbone, a phosphorodiamidate mopholino (PMO) backbone, a peptide nucleic acid backbone, a pseudopeptide backbone, or a combination thereof,
or any combination of the foregoing.

7. (canceled)

8. (canceled)

9. (canceled)

10. The ASO of claim 1, wherein:
a) at least 10%, at least 20%, at least 30%, at least 50%, at least 80% or 100% of internucleotide linkages of the ASO are phosphorothioate internucleotide linkages;
b) at least 10%, at least 20%, at least 30%, at least 50%, at least 80% or 100% of internucleotide linkages of the ASO are phosphodiester internucleotide linkages;
c) no more than 90%, no more than 80%, no more than 70%, no more than 50%, or no more than 20% of the internucleotide linkages of the ASO are phosphorothioate internucleotide linkages; or
d) no more than 90%, no more than 80%, no more than 70%, no more than 50%, or no more than 20% of the internucleotide linkages of the ASO are phosphodiester internucleotide linkages.

11. (canceled)

12. (canceled)

13. (canceled)

14. (canceled)

15. (canceled)

16. (canceled)

17. (canceled)

18. The ASO of claim 6, wherein at least 10%, at least 20%, at least 30%, at least 50%, at least 80% or 100% of riboses or deoxyriboses of the ASO comprise a 2′-O-methoxyethyl ribose sugar.

19. The ASO of claim 1, wherein the ASO comprises a nucleotide sequence having 85-100% sequence identity to at least one sequence set forth in SEQ ID NOs:1-11, 43-50, and 51-75.

20. (canceled)

21. (canceled)

22. (canceled)

23. (canceled)

24. (canceled)

25. The ASO of claim 1, wherein the ASO comprises
(eC)#(eC)#(eT)#(eC)#(eG)#(eC)#(eC)#(eC)#(eA)#(eG)#(eA)#(eA)#(eC)#(eA)#(e G)#(eT)#(eG)#(eG)#(eA),
(eC)#(eC)#(eU)#(eC)#(eG)#(eC)#(eC)#(eC)#(eA)#(eG)#(eA)#(eA)#(eC)#(eA)#(eG) #(eU)#(eG)#(eG)#(eA),
(eC)#(eC)#(eT)#(eC)(eG)(eC)(eC)#(eC)#(eA)#(eG)#(eA)#(eA)#(eC)(eA)(eG)(eT) #(eG)#(eG)#(eA), and/or
(eC)#(eC)#(eU)#(eC)(eG)(eC)(eC)#(eC)#(eA)#(eG)#(eA)#(eA)#(eC)(eA)(eG)(e U)#(eG)#(eG)#(eA),
wherein e is a 2′-O-methoxyethyl ribose sugar, and wherein # is a phosphorothioate internucleotide linkage.

26. (canceled)

27. A pharmaceutical composition, comprising at least one ASO of claim 1 and a pharmaceutically acceptable excipient, diluent, and/or carrier.

28. (canceled)

29. A method of treating a disease in a subject in need thereof, comprising administering to the subject a therapeutically effective amount of the pharmaceutical composition of claim 27.

30. The method of claim 29, wherein the disease is a fragile X-associated disorder.

31. The method of claim 30, wherein the fragile X-associated disorder is fragile X syndrome (FXS), fragile X-associated primary ovarian insufficiency (FXPOI), or fragile X-associated tremor/ataxia syndrome (FXTAS).

32. (canceled)

33. The method of claim 29, wherein the therapeutically effective amount of the pharmaceutical composition decreases an aberrant FMR1 transcript, decreases a protein encoded by the aberrant FMR1 transcript, increases expression of fragile X messenger ribonucleoprotein (FMRP), or a combination thereof.

34. The method of claim 33, wherein the aberrant FMR1 transcript comprises a FMR1-217 transcript.

35. (canceled)

36. (canceled)

37. (canceled)

38. A method of reducing a FMR1-217 transcript in a cell, comprising contacting the cell with an effective amount of at least one ASO of claim 1.

39. (canceled)

40. The method of claim 38, wherein the effective amount of the at least one ASO decreases the FMR1-217 transcript by at least 25%.

41. The method of claim 38, wherein the effective amount of the at least one ASO increases expression of fragile X messenger ribonucleoprotein (FMRP).

42. (canceled)

43. The method of claim 38, wherein the cell is a cell derived from or in a subject having a fragile X-associated disorder.

44. The method of claim 43, wherein the fragile X-associated disorder is fragile X syndrome (FXS), fragile X-associated primary ovarian insufficiency (FXPOI), or fragile X-associated tremor/ataxia syndrome (FXTAS).

45. (canceled)


SEQUENCE LISTING (117 sequences)

SEQ ID NO: 1 - DNA, 20 bp, synthetic construct
  misc_feature 1..20: note = W-704
  source 1..20: mol_type = other DNA
agaagccaaaggagacctga

SEQ ID NO: 2 - DNA, 20 bp, synthetic construct
  misc_feature 1..20: note = W-705
  source 1..20: mol_type = other DNA
aaagagaagccaaaggagac

SEQ ID NO: 3 - DNA, 22 bp, synthetic construct
  misc_feature 1..22: note = W-706
  source 1..22: mol_type = other DNA
ctagaccggaaaagagaagcca

SEQ ID NO: 4 - DNA, 21 bp, synthetic construct
  misc_feature 1..21: note = W-707
  source 1..21: mol_type = other DNA
atgctagaccggaaaagagaa

SEQ ID NO: 5 - DNA, 20 bp, synthetic construct
  misc_feature 1..20: note = W-708
  source 1..20: mol_type = other DNA
caatgctagaccggaaaaga

SEQ ID NO: 6 - DNA, 21 bp, synthetic construct
  misc_feature 1..21: note = W-709
  source 1..21: mol_type = other DNA
aagtcccaatgctagaccgga

SEQ ID NO: 7 - DNA, 20 bp, synthetic construct
  misc_feature 1..20: note = W-710
  source 1..20: mol_type = other DNA
tctccgaagtcccaatgcta

SEQ ID NO: 8 - DNA, 18 bp, synthetic construct
  misc_feature 1..18: note = W-711
  source 1..18: mol_type = other DNA
gagctctccgaagtccca

SEQ ID NO: 9 - DNA, 20 bp, synthetic construct
  misc_feature 1..20: note = W-712
  source 1..20: mol_type = other DNA
agaacagtggagctctccga

SEQ ID NO: 10 - DNA, 20 bp, synthetic construct
  misc_feature 1..20: note = W-713
  source 1..20: mol_type = other DNA
cgcccagaacagtggagctc

SEQ ID NO: 11 - DNA, 20 bp, synthetic construct
  misc_feature 1..20: note = W-714
  source 1..20: mol_type = other DNA
cctcgcccagaacagtggag

SEQ ID NO: 12 - DNA, 20 bp, synthetic construct
  misc_feature 1..20: note = Iso1_1Forward (Iso1_1 F) Primer
  source 1..20: mol_type = other DNA
agaagatggaggagctggtg

SEQ ID NO: 13 - DNA, 20 bp, synthetic construct
  misc_feature 1..20: note = Iso12_1Reverse (Iso12_1 R) Primer
  source 1..20: mol_type = other DNA
cagtggagctctccgaagtc

SEQ ID NO: 14 - DNA, 20 bp, synthetic construct
  misc_feature 1..20: note = Iso12_2Forward Primer
  source 1..20: mol_type = other DNA
ccagcagtgcattgaagaag

SEQ ID NO: 15 - DNA, 20 bp, synthetic construct
  misc_feature 1..20: note = Iso12_2Reverse Primer
  source 1..20: mol_type = other DNA
ctgaagcatgtgcattcctg

SEQ ID NO: 16 - DNA, 24 bp, synthetic construct
  misc_feature 1..24: note = Iso1_1 Reverse (Iso1_1 R) Primer
  source 1..24: mol_type = other DNA
ttcatgaacatcctttacaaatgc

SEQ ID NO: 17 - DNA, 18 bp, synthetic construct
  misc_feature 1..18: note = Exon1 Forward (Exon1 F) Primer
  source 1..18: mol_type = other DNA
tagcagggctgaagagaa

SEQ ID NO: 18 - DNA, 19 bp, synthetic construct
  misc_feature 1..19: note = Exon1 Reverse (Exon1 R) Primer
  source 1..19: mol_type = other DNA
cttgtagaaagcgccattg

SEQ ID NO: 19 - DNA, 312 bp, Homo sapiens
  source 1..312: mol_type = genomic DNA
ctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaag

SEQ ID NO: 20 - DNA, 53 bp, Homo sapiens
  source 1..53: mol_type = genomic DNA
gcatttgtaaaggatgttcatgaagattcaataacagttgcatttgaaaacaa

SEQ ID NO: 21 - DNA, 1724 bp, Homo sapiens
  source 1..1724: mol_type = genomic DNA
cattgggacttcggagagctccactgttctgggcgagggctgtgaagaaagagtagtaagaagcggtagtcggcaccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttcagcgtctaaaaccattagcagcgctgctacttaaaaattgtgtgtgtgtgtttaagtttccaaagacctaaatatatgccatgaaacttcaggtaattaactgagagtatattattactagggcattttttttttaactgagcgaaaatatttttgtgcccctaagaacttgaccacatttcctttgaatttgtggtgttgcagtggactgaattgttgaggctttatataggcattcatgggtttactgtgctttttaaagttacaccattgcagatcaactaacacctttcagttttaaaaggaagatttacaaatttgatgtagcagtagtgcgtttgttggtatgtaggtgctgtataaattcatctataaattctcatttccttttgaatgtctataacctctttcaataatatcccaccttactacagtattttggcaatagaaggtgcgtgtggaaggaaggctggaaaatagctattagcagtgtccaacacaattcttaaatgtattgtagaatggcttgaatgtttcagacaggacacgtttggctataggaaaataaacaattgactttattctgtgtttaccaattttatgaagacatttggagatcagtatatttcataaatgagtaaagtatgtaaactgttccatactttgagcacaaagataaagccttttgctgtaaaaggaggcaaaaggtaaccccgcgtttatgttcttaacagtctcatgaatatgaaattgtttcagttgactctgcagtcaaaattttaatttcattgattttattgatccataatttcttctggtgagtttgcgtagaatcgttcacggtcctagattagtggttttggtcactagatttctggcactaataactataatacatatacatatatatgtgtgagtaacggctaatggttaggcaagattttgattgacctgtgatataaacttagattggatgccactaaagtttgcttatcacagagggcaagtagcacattatggccttgaagtacttattgttctcttccagcaacttatgatttgctccagtgattttgcttgcacactgactggaatataagaaatgccttctatttttgctattaattccctccttttttgttttgttttgtaacgaagttgtttaacttgaaggtgaatgaagaataggttggttgccccttagttccctgaggagaaatgttaatacttgaacaagtgtgtgtcagacaaattgctgttatgtttatttaattaagtttgatttctaagaaaatctcaaatggtctgcactgatggaagaacagtttctgtaacaaaaaagcttgaaatttttatatgacttataatactgctgtgagttttaaaagtaaagcaaaagtaaactgagttgcttgtccagtgggatggacaggaaagatgtgaaataaaaaccaatgaaaaatgaa

SEQ ID NO: 22 - protein, 31 aa, Homo sapiens
MEELVVEVRGSNGAFYKHWDFGELHCSGRGL

SEQ ID NO: 23 - DNA, 108 bp, Homo sapiens
  source 1..108: mol_type = genomic DNA
cgcccgcagcccacctctcgggggcgggctcccggcgctagcagggctgaagagaagatggaggagctggtggtggaagtgcggggctccaatggcgctttctacaag

SEQ ID NO: 24 - RNA, 86 nt, synthetic construct
  misc_feature 1..86: note = ASO target
  source 1..86: mol_type = other RNA
tcaggtctcctttggcttctcttttccggtctagcattgggacttcggagagctccactgttctgggcgagggctgtgaagaaaga

SEQ ID NO: 25 - RNA, 51 nt, synthetic construct
  misc_feature 1..51: note = ASO target
  source 1..51: mol_type = other RNA
tcaggtctcctttggcttctcttttccggtctagcattgggacttcggaga

SEQ ID NO: 26 - RNA, 52 nt, synthetic construct
  misc_feature 1..52: note = ASO target
  source 1..52: mol_type = other RNA
cattgggacttcggagagctccactgttctgggcgagggctgtgaagaaaga

SEQ ID NO: 27 - RNA, 46 nt, synthetic construct
  misc_feature 1..46: note = ASO target
  source 1..46: mol_type = other RNA
tgggacttcggagagctccactgttctgggcgagggctgtgaagaa

SEQ ID NO: 28 - RNA, 20 nt, synthetic construct
  misc_feature 1..20: note = ASO target
  source 1..20: mol_type = other RNA
tcaggtctcctttggcttct

SEQ ID NO: 29 - RNA, 20 nt, synthetic construct
  misc_feature 1..20: note = ASO target
  source 1..20: mol_type = other RNA
gtctcctttggcttctcttt

SEQ ID NO: 30 - RNA, 22 nt, synthetic construct
  misc_feature 1..22: note = ASO target
  source 1..22: mol_type = other RNA
tggcttctcttttccggtctag

SEQ ID NO: 31 - RNA, 21 nt, synthetic construct
  misc_feature 1..21: note = ASO target
  source 1..21: mol_type = other RNA
ttctcttttccggtctagcat

SEQ ID NO: 32 - RNA, 20 nt, synthetic construct
  misc_feature 1..20: note = ASO target
  source 1..20: mol_type = other RNA
tcttttccggtctagcattg

SEQ ID NO: 33 - RNA, 21 nt, synthetic construct
  misc_feature 1..21: note = ASO target
  source 1..21: mol_type = other RNA
tccggtctagcattgggactt

SEQ ID NO: 34 - RNA, 20 nt, synthetic construct
  misc_feature 1..20: note = ASO target
  source 1..20: mol_type = other RNA
tagcattgggacttcggaga

SEQ ID NO: 35 - RNA, 18 nt, synthetic construct
  misc_feature 1..18: note = ASO target
  source 1..18: mol_type = other RNA
tgggacttcggagagctc

SEQ ID NO: 36 - RNA, 20 nt, synthetic construct
  misc_feature 1..20: note = ASO target
  source 1..20: mol_type = other RNA
tcggagagctccactgttct

SEQ ID NO: 37 - RNA, 20 nt, synthetic construct
  misc_feature 1..20: note = ASO target
  source 1..20: mol_type = other RNA
gagctccactgttctgggcg

SEQ ID NO: 38 - RNA, 20 nt, synthetic construct
  misc_feature 1..20: note = ASO target
  source 1..20: mol_type = other RNA
ctccactgttctgggcgagg

SEQ ID NO: 39 - RNA, 21 nt, synthetic construct
  misc_feature 1..21: note = ASO target
  source 1..21: mol_type = other RNA
ggacttcggagagctccactg

SEQ ID NO: 40 - RNA, 21 nt, synthetic construct
  misc_feature 1..21: note = ASO target
  source 1..21: mol_type = other RNA
ggagagctccactgttctggg

SEQ ID NO: 41 - RNA, 20 nt, synthetic construct
  misc_feature 1..20: note = ASO target
  source 1..20: mol_type = other RNA
tgttctgggcgagggctgtg

SEQ ID NO: 42 - RNA, 20 nt, synthetic construct
  misc_feature 1..20: note = ASO target
  source 1..20: mol_type = other RNA
tgggcgagggctgtgaagaa

SEQ ID NO: 43 - DNA, 21 bp, synthetic construct
  misc_feature 1..21: note = ASO target
  source 1..21: mol_type = other DNA
cagtggagctctccgaagtcc

SEQ ID NO: 44 - DNA, 21 bp, synthetic construct
  misc_feature 1..21: note = ASO target
  source 1..21: mol_type = other DNA
cccagaacagtggagctctcc

SEQ ID NO: 45 - DNA, 20 bp, synthetic construct
  misc_feature 1..20: note = ASO target
  source 1..20: mol_type = other DNA
cacagccctcgcccagaaca

SEQ ID NO: 46 - DNA, 20 bp, synthetic construct
  misc_feature 1..20: note = ASO target
  source 1..20: mol_type = other DNA
ttcttcacagccctcgccca

SEQ ID NO: 47 - DNA, 86 bp, synthetic construct
  misc_feature 1..86: note = ASO target
  source 1..86: mol_type = other DNA
tctttcttcacagccctcgcccagaacagtggagctctccgaagtcccaatgctagaccggaaaagagaagccaaaggagacctga

SEQ ID NO: 48 - DNA, 51 bp, synthetic construct
  misc_feature 1..51: note = ASO target
  source 1..51: mol_type = other DNA
tctccgaagtcccaatgctagaccggaaaagagaagccaaaggagacctga

SEQ ID NO: 49 - DNA, 52 bp, synthetic construct
  misc_feature 1..52: note = ASO target
  source 1..52: mol_type = other DNA
tctttcttcacagccctcgcccagaacagtggagctctccgaagtcccaatg

SEQ ID NO: 50 - DNA, 46 bp, synthetic construct
  misc_feature 1..46: note = ASO target
  source 1..46: mol_type = other DNA
ttcttcacagccctcgcccagaacagtggagctctccgaagtccca

SEQ ID NO: 51 - RNA, 20 nt, synthetic construct
  misc_feature 1..20: note = ASO
  source 1..20: mol_type = other RNA
agaagccaaaggagacctga

SEQ ID NO: 52 - RNA, 20 nt, synthetic construct
  misc_feature 1..20: note = ASO
  source 1..20: mol_type = other RNA
aaagagaagccaaaggagac

SEQ ID NO: 53 - RNA, 22 nt, synthetic construct
  misc_feature 1..22: note = ASO
  source 1..22: mol_type = other RNA
ctagaccggaaaagagaagcca

SEQ ID NO: 54 - RNA, 21 nt, synthetic construct
  misc_feature 1..21: note = ASO
  source 1..21: mol_type = other RNA
atgctagaccggaaaagagaa

SEQ ID NO: 55 - RNA, 20 nt, synthetic construct
  misc_feature 1..20: note = ASO
  source 1..20: mol_type = other RNA
caatgctagaccggaaaaga

SEQ ID NO: 56 - RNA, 21 nt, synthetic construct
  misc_feature 1..21: note = ASO
  source 1..21: mol_type = other RNA
aagtcccaatgctagaccgga

SEQ ID NO: 57 - RNA, 20 nt, synthetic construct
  misc_feature 1..20: note = ASO
  source 1..20: mol_type = other RNA
tctccgaagtcccaatgcta

SEQ ID NO: 58 - RNA, 18 nt, synthetic construct
  misc_feature 1..18: note = ASO
  source 1..18: mol_type = other RNA
gagctctccgaagtccca

SEQ ID NO: 59 - RNA, 20 nt, synthetic construct
  misc_feature 1..20: note = ASO
  source 1..20: mol_type = other RNA
agaacagtggagctctccga

SEQ ID NO: 60 - RNA, 20 nt, synthetic construct
  misc_feature 1..20: note = ASO
  source 1..20: mol_type = other RNA
cgcccagaacagtggagctc

SEQ ID NO: 61 - RNA, 20 nt, synthetic construct
  misc_feature 1..20: note = ASO
  source 1..20: mol_type = other RNA
cctcgcccagaacagtggag

SEQ ID NO: 62 - RNA, 21 nt, synthetic construct
  misc_feature 1..21: note = ASO
  source 1..21: mol_type = other RNA
cagtggagctctccgaagtcc

SEQ ID NO: 63 - RNA, 21 nt, synthetic construct
  misc_feature 1..21: note = ASO
  source 1..21: mol_type = other RNA
cccagaacagtggagctctcc

SEQ ID NO: 64 - RNA, 20 nt, synthetic construct
  misc_feature 1..20: note = ASO
  source 1..20: mol_type = other RNA
cacagccctcgcccagaaca

SEQ ID NO: 65 - RNA, 20 nt, synthetic construct
  misc_feature 1..20: note = ASO
  source 1..20: mol_type = other RNA
ttcttcacagccctcgccca

SEQ ID NO: 66 - RNA, 86 nt, synthetic construct
  misc_feature 1..86: note = ASO
  source 1..86: mol_type = other RNA
tctttcttcacagccctcgcccagaacagtggagctctccgaagtcccaatgctagaccggaaaagagaagccaaaggagacctga

SEQ ID NO: 67 - RNA, 51 nt, synthetic construct
  misc_feature 1..51: note = ASO
  source 1..51: mol_type = other RNA
tctccgaagtcccaatgctagaccggaaaagagaagccaaaggagacctga

SEQ ID NO: 68 - RNA, 52 nt, synthetic construct
  misc_feature 1..52: note = ASO
  source 1..52: mol_type = other RNA
tctttcttcacagccctcgcccagaacagtggagctctccgaagtcccaatg

SEQ ID NO: 69 - RNA, 46 nt, synthetic construct
  misc_feature 1..46: note = ASO
  source 1..46: mol_type = other RNA
ttcttcacagccctcgcccagaacagtggagctctccgaagtccca

SEQ ID NO: 70 - DNA, 23 bp, synthetic construct
  source 1..23: mol_type = other DNA
cctcgcccagaacagtggagctc

SEQ ID NO: 71 - DNA, 19 bp, synthetic construct
  source 1..19: mol_type = other DNA
gcccagaacagtggagctc

SEQ ID NO: 72 - DNA, 19 bp, synthetic construct
  source 1..19: mol_type = other DNA
cctcgcccagaacagtgga

SEQ ID NO: 73 - RNA, 23 nt, synthetic construct
  source 1..23: mol_type = other RNA
cctcgcccagaacagtggagctc

SEQ ID NO: 74 - RNA, 19 nt, synthetic construct
  source 1..19: mol_type = other RNA
gcccagaacagtggagctc

SEQ ID NO: 75 - RNA, 19 nt, synthetic construct
  source 1..19: mol_type = other RNA
cctcgcccagaacagtgga

SEQ ID NO: 76 - DNA, 20 bp, synthetic construct
  source 1..20: mol_type = other DNA
  modified_base 1..20: mod_base = OTHER; note = 2'-O-methoxyethyl nucleotide
agaagccaaaggagacctga

SEQ ID NO: 77 - RNA, 20 nt, synthetic construct
  source 1..20: mol_type = other RNA
  modified_base 1..20: mod_base = OTHER; note = 2'-O-methoxyethyl nucleotide
agaagccaaaggagacctga

SEQ ID NO: 78 - DNA, 20 bp, synthetic construct
  source 1..20: mol_type = other DNA
  modified_base 1..20: mod_base = OTHER; note = 2'-O-methoxyethyl nucleotide
aaagagaagccaaaggagac

SEQ ID NO: 79 - RNA, 20 nt, synthetic construct
  source 1..20: mol_type = other RNA
  modified_base 1..20: mod_base = OTHER; note = 2'-O-methoxyethyl nucleotide
aaagagaagccaaaggagac

SEQ ID NO: 80 - DNA, 22 bp, synthetic construct
  source 1..22: mol_type = other DNA
  modified_base 1..22: mod_base = OTHER; note = 2'-O-methoxyethyl nucleotide
ctagaccggaaaagagaagcca

SEQ ID NO: 81 - RNA, 22 nt, synthetic construct
  source 1..22: mol_type = other RNA
  modified_base 1..22: mod_base = OTHER; note = 2'-O-methoxyethyl nucleotide
ctagaccggaaaagagaagcca

SEQ ID NO: 82 - DNA, 21 bp, synthetic construct
  source 1..21: mol_type = other DNA
  modified_base 1..21: mod_base = OTHER; note = 2'-O-methoxyethyl nucleotide
atgctagaccggaaaagagaa

SEQ ID NO: 83 - RNA, 21 nt, synthetic construct
  source 1..21: mol_type = other RNA
  modified_base 1..21: mod_base = OTHER; note = 2'-O-methoxyethyl nucleotide
atgctagaccggaaaagagaa

SEQ ID NO: 84 - DNA, 20 bp, synthetic construct
  source 1..20: mol_type = other DNA
  modified_base 1..20: mod_base = OTHER; note = 2'-O-methoxyethyl nucleotide
caatgctagaccggaaaaga

SEQ ID NO: 85 - RNA, 20 nt, synthetic construct
  source 1..20: mol_type = other RNA
  modified_base 1..20: mod_base = OTHER; note = 2'-O-methoxyethyl nucleotide
caatgctagaccggaaaaga

SEQ ID NO: 86 - DNA, 21 bp, synthetic construct
  source 1..21: mol_type = other DNA
  modified_base 1..21: mod_base = OTHER; note = 2'-O-methoxyethyl nucleotide
aagtcccaatgctagaccgga

SEQ ID NO: 87 - RNA, 21 nt, synthetic construct
  source 1..21: mol_type = other RNA
  modified_base 1..21: mod_base = OTHER; note = 2'-O-methoxyethyl nucleotide
aagtcccaatgctagaccgga

SEQ ID NO: 88 - DNA, 20 bp, synthetic construct
  source 1..20: mol_type = other DNA
  modified_base 1..20: mod_base = OTHER; note = 2'-O-methoxyethyl nucleotide
tctccgaagtcccaatgcta

SEQ ID NO: 89 - RNA, 20 nt, synthetic construct
  source 1..20: mol_type = other RNA
  modified_base 1..20: mod_base = OTHER; note = 2'-O-methoxyethyl nucleotide
tctccgaagtcccaatgcta

SEQ ID NO: 90 - DNA, 18 bp, synthetic construct
  source 1..18: mol_type = other DNA
  modified_base 1..18: mod_base = OTHER; note = 2'-O-methoxyethyl nucleotide
gagctctccgaagtccca

SEQ ID NO: 91 - RNA, 18 nt, synthetic construct
  source 1..18: mol_type = other RNA
  modified_base 1..18: mod_base = OTHER; note = 2'-O-methoxyethyl nucleotide
gagctctccgaagtccca

SEQ ID NO: 92 - DNA, 20 bp, synthetic construct
  source 1..20: mol_type = other DNA
  modified_base 1..20: mod_base = OTHER; note = 2'-O-methoxyethyl nucleotide
agaacagtggagctctccga

SEQ ID NO: 93 - RNA, 20 nt, synthetic construct
  source 1..20: mol_type = other RNA
  modified_base 1..20: mod_base = OTHER; note = 2'-O-methoxyethyl nucleotide
agaacagtggagctctccga

SEQ ID NO: 94 - DNA, 20 bp, synthetic construct
  source 1..20: mol_type = other DNA
  modified_base 1..20: mod_base = OTHER; note = 2'-O-methoxyethyl nucleotide
cgcccagaacagtggagctc

SEQ ID NO: 95 - RNA, 20 nt, synthetic construct
  source 1..20: mol_type = other RNA
  modified_base 1..20: mod_base = OTHER; note = 2'-O-methoxyethyl nucleotide
cgcccagaacagtggagctc

SEQ ID NO: 96 - DNA, 20 bp, synthetic construct
  source 1..20: mol_type = other DNA
  modified_base 1..20: mod_base = OTHER; note = 2'-O-methoxyethyl nucleotide
cctcgcccagaacagtggag

SEQ ID NO: 97 - RNA, 20 nt, synthetic construct
  source 1..20: mol_type = other RNA
  modified_base 1..20: mod_base = OTHER; note = 2'-O-methoxyethyl nucleotide
cctcgcccagaacagtggag

SEQ ID NO: 98 - DNA, 16 bp, synthetic construct
  source 1..16: mol_type = other DNA
  modified_base 1..3: mod_base = OTHER; note = locked nucleic acid
  modified_base 14..16: mod_base = OTHER; note = locked nucleic acid
aagctgcactgtgctg

SEQ ID NO: 99 - RNA, 16 nt, synthetic construct
  source 1..16: mol_type = other RNA
  modified_base 1..3: mod_base = OTHER; note = locked nucleic acid
  modified_base 14..16: mod_base = OTHER; note = locked nucleic acid
aagctgcactgtgctg

SEQ ID NO: 100 - RNA, 23 nt, synthetic construct
  source 1..23: mol_type = other RNA
  modified_base 1..23: mod_base = OTHER; note = 2'-O-methoxyethyl nucleotide
cctcgcccagaacagtggagctc

SEQ ID NO: 101 - DNA, 23 bp, synthetic construct
  source 1..23: mol_type = other DNA
  modified_base 1..23: mod_base = OTHER; note = 2'-O-methoxyethyl nucleotide
cctcgcccagaacagtggagctc

SEQ ID NO: 102 - RNA, 20 nt, synthetic construct
  source 1..20: mol_type = other RNA
  modified_base 1..20: mod_base = OTHER; note = 2'-O-methoxyethyl nucleotide
cgcccagaacagtggagctc

SEQ ID NO: 103 - DNA, 20 bp, synthetic construct
  source 1..20: mol_type = other DNA
  modified_base 1..20: mod_base = OTHER; note = 2'-O-methoxyethyl nucleotide
cgcccagaacagtggagctc

SEQ ID NO: 104 - RNA, 19 nt, synthetic construct
  source 1..19: mol_type = other RNA
  modified_base 1..19: mod_base = OTHER; note = 2'-O-methoxyethyl nucleotide
gcccagaacagtggagctc

SEQ ID NO: 105 - DNA, 19 bp, synthetic construct
  source 1..19: mol_type = other DNA
  modified_base 1..19: mod_base = OTHER; note = 2'-O-methoxyethyl nucleotide
gcccagaacagtggagctc

SEQ ID NO: 106 - RNA, 19 nt, synthetic construct
  source 1..19: mol_type = other RNA
  modified_base 1..19: mod_base = OTHER; note = 2'-O-methoxyethyl nucleotide
cctcgcccagaacagtgga

SEQ ID NO: 107 - DNA, 19 bp, synthetic construct
  source 1..19: mol_type = other DNA
  modified_base 1..19: mod_base = OTHER; note = 2'-O-methoxyethyl nucleotide
cctcgcccagaacagtgga

SEQ ID NO: 108 - RNA, 19 nt, synthetic construct
  source 1..19: mol_type = other RNA
  modified_base 1..19: mod_base = OTHER; note = 2'-O-methoxyethyl nucleotide
cctcgcccagaacagtgga

SEQ ID NO: 109 - DNA, 19 bp, synthetic construct
  source 1..19: mol_type = other DNA
  modified_base 1..19: mod_base = OTHER; note = 2'-O-methoxyethyl nucleotide
cctcgcccagaacagtgga

SEQ ID NO: 110 - RNA, 21 nt, synthetic construct
  source 1..21: mol_type = other RNA
  modified_base 1..21: mod_base = OTHER; note = 2'-O-methoxyethyl nucleotide
cagtggagctctccgaagtcc

SEQ ID NO: 111 - DNA, 21 bp, synthetic construct
  source 1..21: mol_type = other DNA
  modified_base 1..21: mod_base = OTHER; note = 2'-O-methoxyethyl nucleotide
cagtggagctctccgaagtcc

SEQ ID NO: 112 - RNA, 21 nt, synthetic construct
  source 1..21: mol_type = other RNA
  modified_base 1..21: mod_base = OTHER; note = 2'-O-methoxyethyl nucleotide
cccagaacagtggagctctcc

SEQ ID NO: 113 - DNA, 21 bp, synthetic construct
  source 1..21: mol_type = other DNA
  modified_base 1..21: mod_base = OTHER; note = 2'-O-methoxyethyl nucleotide
cccagaacagtggagctctcc

SEQ ID NO: 114 - RNA, 20 nt, synthetic construct
  source 1..20: mol_type = other RNA
  modified_base 1..20: mod_base = OTHER; note = 2'-O-methoxyethyl nucleotide
cacagccctcgcccagaaca

SEQ ID NO: 115 - DNA, 20 bp, synthetic construct
  source 1..20: mol_type = other DNA
  modified_base 1..20: mod_base = OTHER; note = 2'-O-methoxyethyl nucleotide
cacagccctcgcccagaaca

SEQ ID NO: 116 - RNA, 20 nt, synthetic construct
  source 1..20: mol_type = other RNA
  modified_base 1..20: mod_base = OTHER; note = 2'-O-methoxyethyl nucleotide
ttcttcacagccctcgccca

SEQ ID NO: 117 - DNA, 20 bp, synthetic construct
  source 1..20: mol_type = other DNA
  modified_base 1..20: mod_base = OTHER; note = 2'-O-methoxyethyl nucleotide
ttcttcacagccctcgccca